(19) United States
(12) Patent Application Publication
(10) Pub. No.: US 2025/0041385 A1
(43) Pub. Date: Feb. 6, 2025

(54) MODIFIED ACTRII PROTEINS AND METHODS OF USE THEREOF

(71) Applicant: BIOGEN MA INC.

(72) Inventors: R. Blake PEPINSKY, Arlington, MA (US); Joseph Walter ARNDT, Swampscott, MA (US); Isin DALKILIC-LIDDLE, Watertown, MA (US); YuTing LIU, Belmont, MA (US); Benjamin Andrew SMITH, Belmont, MA (US)

(73) Assignee: BIOGEN MA INC., (US)

(21) Appl. No.: 18/717,939

(22) PCT Filed: Dec. 9, 2022
(86) PCT No.: PCT/US2022/081293

§ 371 Date: Jun. 7, 2024

Related U.S. Application Data
(60) Provisional application No. 63/288,249, filed on Dec. 10, 2021.

(51) Int. Cl. A61K 38/17; C07K 14/71

(52) U.S. Cl. CPC A61K 38/179; C07K 14/71; C07K 2318/10

(57) ABSTRACT

An isolated protein comprising a mutant soluble activin II receptor (ActRIIA or ActRIIB) extracellular domain (Ac-tRIIA-ECD or ActRIIB-ECD), wherein said mutant soluble ActRIIA-ECD or ActRIIB-ECD comprises a mutation to remove the N-linked glycosylation site corresponding to position N18 of SEQ ID NO: 1.

### CROSS-REFERENCE TO RELATED APPLICATIONS

This application claims the benefit of U.S. Provisional Application No. 63/288,249, filed Dec. 10, 2021. The entire contents of the above-identified application is hereby fully incorporated herein by reference.

### REFERENCE TO AN ELECTRONIC SEQUENCE LISTING

The contents of the electronic sequence listing (2790-0264-PCT.xml; Size: 1,669,055 bytes; and Date of Creation: Dec. 2, 2022) is herein incorporated by reference in its entirety.

### TECHNICAL FIELD

The subject matter disclosed herein is generally directed to proteins comprising modified ActRIIB and modified ActRIIA proteins and methods of using thereof.

### BACKGROUND

Muscle wasting refers to the progressive loss of muscle mass and/or to the progressive weakening and degeneration of muscles, including skeletal or voluntary muscles, cardiac muscles controlling the heart (cardiomyopathies), and smooth muscles. Chronic muscle wasting is a condition (i.e., persisting over a long period of time) characterized by progressive loss of muscle mass, as well as muscle weakening and degeneration. The loss of muscle mass occurs when the rate of muscle protein degradation exceeds muscle protein synthesis.

Muscle wasting is a debilitating and life-threatening disease state, which has been associated with the development of a number of chronic, neurological, genetic, inflammatory, fibrotic or infectious pathologies, including, e.g., muscular dystrophies, amyotrophic lateral sclerosis, myositis, denervation muscle atrophies, anorexia-cachexia syndrome, cancers, rheumatoid arthritis, osteoarthritis, insulin resistance/diabetes, sarcopenic obesity, age-related sarcopenia, androgen deprivation, corticosteroid myopathy, inflammatory bowel disease, liver cirrhosis, chronic obstructive pulmonary disease, pulmonary fibrosis, chronic renal disease, trauma, cardiomyopathy, chronic heart failure and HIV infection. Other conditions said to cause muscle wasting include chronic lower back pain, advanced age, damage to the central nervous system, peripheral nerve injury, chemical injury, extended burns, hip/knee replacement, disuse atrophy, exposure to microgravity, and long-term hospitalization.

Bone disease is considered any affliction that involves the skeletal system. Bone diseases can be very serious, and require prompt and effective treatment. Bone diseases can be very painful and can rob the patient of mobility and independence. While the causative factors vary by disease, many bone diseases are caused by, e.g., genetic factors, viral infection, chemical abnormalities, lack of bone collagen, injuries, fractures, damage to blood vessel, excessive use of alcohol, or the long-term use of certain medications. Examples of bone disease include osteoporosis, osteomalacia, osteogenesis imperfecta, fibrous dysplasia, ossificans progressiva, corticosteroid-induced bone loss, bone fracture, bone metastasis and Paget's disease of the bone.

Activin IIA receptor (ActRIIA) and Activin IIB receptor (ActRIIB) are type II receptors for a subset of TGF-β family member ligands, including, e.g., activin A, myostatin (also known as GDF-8), growth differentiation factor-11 (GDF-11), and various other bone morphogenetic proteins (BMPs) such as BMP-3, BMP-6, BMP-9 (also known as GDF-2) and BMP-10. ActRIIA and ActRIIB have been identified as the type II receptors for activins, including activin A, activin B and activin AB. ActRIIA and ActRIIB are referred to generically herein as “ActRII” proteins. ActRIIB is a high affinity receptor for myostatin, a key negative regulator of muscle growth, and thus plays central role in controlling muscle mass. The binding of these ligands to ActRIIA and/or ActRIIB can regulate cell differentiation, apoptosis, protein synthesis and degradation, mineralization, hematopoiesis, angiogenesis, steroid synthesis, adhesion, migration, extracellular matrix production and fibrogenesis. The specific response depends upon the types and levels of the TGF-B ligands and receptors as well as the cellular state and environment. The ActRIIB signaling pathway mediates cellular responses via Smad2/3 transcription factors, and activation of the ActRIIB signaling pathway has been implicated in pathogenesis and progression of many diseases including muscle wasting, bone loss, fibrosis and inflammation. Several members of the TGF-B family, including myostatin, activins and GDF11, mediate Smad2/3 activation by coupling to ActRIIB.

Previous studies have shown that pharmacological sequestration of these ligands with ActRIIB-Fc leads to profound muscle growth and bone anabolism in animal models, demonstrating about three times more muscle growth than myostatin-neutralizing antibody in normal mice, and demonstrating the ability to further stimulate significant muscle gain in myostatin null mice. In addition to its marked anabolic effects on muscle and bone, ActRIIB-Fc has also been shown to have important anti-fibrosis and anti-inflammatory effects in preclinical models and various ActRIIA-Fc and ActRIIB-Fc fusion proteins have been, or are currently being clinically evaluated in patients for the treatment of muscle wasting disorders and/or bone diseases associated with the development of a number of chronic, neurological, genetic, inflammatory, fibrotic or infectious pathologies.

However, while there have been important advancements, there still exists a critical need to provide novel therapeutics, which are both highly effective and safe, for the treatment of muscle wasting and/or bone disease associated with the development of a number of chronic, neurological, genetic, inflammatory, fibrotic or infectious pathologies.

Citation or identification of any document in this application is not an admission that such document is available as prior art to the present invention.

### SUMMARY

In one aspect, the present disclosure provides an isolated protein comprising a mutant soluble activin IIB receptor (ActRIIB) extracellular domain (ActRIIB-ECD), or a mutant soluble activin IIA receptor (ActRIIA) extracellular domain (ActRIIA-ECD), wherein the mutant soluble ActRII-ECD comprises a mutation to remove the N-linked glycosylation site corresponding to position N18 of SEQ ID NO: 1.

In some embodiments, the mutant soluble ActRIIB-ECD further comprises a substitution of at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 with another amino acid. In some embodiments, the mutant soluble ActRIIB-ECD comprises an amino acid sequence at least 95% identical to an amino acid sequence selected from the group consisting of SEQ ID NOs: 1, 3-37, 51-117, 327, 330, and 333, wherein the asparagine at position N18 is substituted with another amino acid. In some embodiments, the mutant soluble ActRIIB-ECD comprises an amino acid sequence selected from the group consisting of SEQ ID NOs: 1, 3-37, 51-117, 327, 330, and 333, wherein the asparagine at position N18 is substituted with another amino acid. In some embodiments, the serine at position S20 of SEQ ID NO: 1 is substituted with an amino acid that is not serine(S) or threonine (T). In some embodiments, the asparagine at position N18 is substituted with glutamine (Q).

In some embodiments, the mutant soluble ActRIIB-ECD comprises an amino acid sequence selected from the group consisting of SEQ ID NOs: 119, 120-221, 329, 332, and 335. In some embodiments, the mutant soluble ActRIIB-ECD comprises an amino acid sequence selected from the group consisting of SEQ ID NOs: 336-354. In some embodiments, the mutant soluble ActRIIB-ECD comprises an amino acid sequence selected from the group consisting of SEQ ID NOs: 355-372. In some embodiments, the mutant soluble ActRIIB-ECD demonstrates increased binding of myostatin relative to an otherwise identical soluble ActRIIB-ECD that includes the N-linked glycosylation site corresponding to position N18 of SEQ ID NO: 1. In some embodiments, the mutant soluble ActRIIB-ECD is glycosylated at an asparagine residue corresponding to position N41 of SEQ ID NO: 1.

In some embodiments, the glycosylated mutant soluble ActRIIB-ECD or ActRIIA-ECD is sialylated. In some embodiments, a sample of the mutant soluble ActRIIB-ECD comprises at least 40% sialylated glycans. In some embodiments, a sample of the mutant soluble ActRII-ECD comprises at least 60% sialylated glycans. In some embodiments, a sample of the mutant soluble ActRII-ECD comprises at least 5%, 10%, 20%, 30%, 40%, 50%, 60%, 70%, 80%, or 90% sialylated glycans at the position corresponding to N41 of SEQ ID NO: 1. In some embodiments, a sample of the mutant soluble ActRII-ECD comprises at least 40%, 45%, 50%, 55%, 60%, 65%, 70%, 75%, 80%, 90% or more sialylated glycans at the position corresponding to N41 of SEQ ID NO: 1.

In some embodiments, the mutant soluble ActRII-ECD is fused to at least one heterologous protein. In some embodiments, the heterologous protein comprises a constant domain of an immunoglobulin. In some embodiments, the heterologous protein comprises an Fc domain of an immunoglobulin. In some embodiments, the Fc domain is selected from the group consisting of the Fc domain of a human immunoglobulin gamma-1 (IgG1), the Fc domain of a human immunoglobulin gamma-2 (IgG2), and the Fc domain of a human immunoglobulin gamma-4 (IgG4). In some embodiments, the mutant soluble ActRIIB-ECD is fused to the heterologous protein by a peptide linker sequence. In some embodiments, the heterologous protein comprises a human Fc domain comprising an amino acid sequence selected from the group consisting of: SEQ ID NO: 39, SEQ ID NO: 41, and SEQ ID NO: 43.

In some embodiments, the isolated protein comprises a linker comprising the amino acid sequence set forth in SEQ ID NO: 44 between the mutant soluble ActRII-ECD and the heterologous protein. In some embodiments, the isolated protein comprises a hinge linker comprising the amino acid sequence set forth in SEQ ID NO: 118 between the mutant soluble ActRII-ECD and the heterologous protein. In some embodiments, the isolated protein comprises a linker comprising the amino acid sequence set forth in SEQ ID NO: 44 and a hinge linker comprising the amino acid sequence set forth in SEQ ID NO: 118 between the mutant soluble ActRII-ECD and the heterologous protein.

In some embodiments, the isolated protein comprises, in an N-terminal to C-terminal direction, the mutant soluble ActRIIECD, the linker of SEQ ID NO: 44, the hinge linker of SEQ ID NO: 118, and the heterologous protein. In some embodiments, the mutant soluble ActRIIB-ECD comprises an amino acid sequence selected from the group consisting of SEQ ID NOs: 119, 120-221, 329, 332,335, 336-354 and 355-372, and wherein the heterologous protein is selected from the group consisting of the Fc domain of a human immunoglobulin gamma-1 (IgG1), the Fc domain of a human immunoglobulin gamma-2 (IgG2), and the Fc domain of a human immunoglobulin gamma-4 (IgG4). In some embodiments, the mutant soluble ActRIIB-ECD comprises SEQ ID NO: 146 and the heterologous protein comprises the Fc domain of a human IgG4. In some embodiments, the isolated protein comprises SEQ ID NO: 222. In some embodiments, the isolated protein consists of SEQ ID NO: 222. In some embodiments, the isolated protein is glycosylated at an asparagine residue in the mutant soluble ActRII-ECD corresponding to positions N41 of SEQ ID NO: 1 and/or an asparagine residue in the Fc domain corresponding to position N67 of SEQ ID NO: 43. In some embodiments, a sample of the mutant soluble ActRII-ECD comprises at least 40% sialylated glycans. In some embodiments, a sample of the mutant soluble ActRII-ECD comprises at least 40% sialylated glycans at the position corresponding to N41 of SEQ ID NO: 1. In some embodiments, a sample of the mutant soluble ActRII-ECD comprises at least 60% sialylated glycans at the position corresponding to N41 of SEQ ID NO: 1.

In another aspect, the present disclosure provides a pharmaceutical composition comprising a therapeutically effective amount of the isolated protein herein in admixture with a pharmaceutically acceptable carrier. In some embodiments, the pharmaceutical composition is formulated for administration by a route selected from the group consisting of: subcutaneous, intramuscular, intravenous, and intrathecal administration. In some embodiments, the pharmaceutical composition further comprises a second agent, wherein the second agent is selected from the group consisting of: growth hormone, ghrelin, IGF1, insulin, prednisone, corticosteroid therapy, androgen-deprivation therapy, anabolic steroids, an antagonist of angiotensin or angiotensin receptor, an antagonist of an inflammatory cytokine such as TNF-alpha, IL-6, IL-1 or their receptors, an antagonist of myostatin, activin A or another member of the TGF-beta family or their receptors (for example and without limitation, an anti-myostatin antibody, an anti-activin antibody), bisphosphonates, RANKL inhibitors, agonists of peroxisome proliferator-activated receptors, β2 agonists, activator of PGC-1alpha, proteasome inhibitors, a cancer therapeutic, a chemotherapeutic agent, a cell therapy, a stem cell therapy, gene therapy, gene targeting therapy, and an antisense oligonucleotide. In some embodiments, the pharmaceutical composition comprises a plurality of the isolated proteins and at least 40% of the isolated proteins are sialylated. In another aspect, the present disclosure provides a sample comprising a plurality of the isolated proteins herein, wherein at least 40% of the isolated proteins are sialylated. In another aspect, a sample of the mutant soluble ActRII-ECD comprises at least 5%, 10%, 20%, 30%, 40%, 50%, 60%, 70%, 80%, or 90% sialylated glycans at the position corresponding to N41 of SEQ ID NO: 1. In another aspect, the present disclosure provides a sample comprising a plurality of the isolated proteins herein, wherein at least 40%, 45%, 50%, 55%, 60%, 65%, 70%, 75%, 80%, 90% or more of the isolated proteins are sialylated at the position corresponding to N41 of SEQ ID NO: 1.

In another aspect, the present disclosure provides a method of treating a myostatin-related or activin A-related disorder in a subject in need thereof, comprising administering a therapeutically effective amount of the pharmaceutical composition herein to the subject. In some embodiments, the myostatin-related or activin A-related disorder is selected from the group consisting of muscle wasting, a bone disorder, a metabolic disorder, and anemia. In some embodiments, the muscle wasting is associate with a condition selected from the group consisting of: muscular dystrophy, myositis, myopathy, motorneuron disease, muscle atrophy, amyotrophic lateral sclerosis, spinal muscular atrophy, neuromuscular junction disease, peripheral nerve disease, spinal cord injury, stroke, neurodegenerative disease, anorexia, cancer, organ failure, trauma, disuse, infection, chronic obstructive pulmonary disease (COPD), sarcopenia, sarcopenic obesity, osteroarthritis, androgen deprivation, emphysema, cystic fibrosis, chronic heart failure, cardiac atrophy, cancer cachexia, renal failure, uremia, protein energy wasting, anorexia, malnutrition, sarcopenia, Acquired Immunodeficiency Syndrome (AIDS), sepsis, burn injury, diabetes, carpal tunnel syndrome, prolonged bed rest, bone fracture, aging, and exposure to microgravity. In some embodiments, the spinal muscular atrophy is selected from the group consisting of infantile progressive spinal muscular atrophy, intermediate spinal muscular atrophy, juvenile spinal muscular atrophy and adult spinal muscular atrophy. In some embodiments, the peripheral nerve disease is selected from the group consisting of Charcot-Marie Tooth disease, Dejerine-Sottas disease and Friedreich's ataxia. In some embodiments, the neurodegenerative disease is selected from the group consisting of Parkinson's disease, Huntington's disease, Alzheimer's disease and Creutzfeldt-Jakob disease. In some embodiments, the aging condition is selected from the group consisting of: frailty of the elderly, age-related sarcopenia, and osteoarthritis. In some embodiments, the motorneuron disease is amyotrophic lateral sclerosis. In some embodiments, the myopathy is critical illness myopathy. In some embodiments, the muscular dystrophy is myotonic dystrophy type 1 (DM1), Facioscapulohumeral muscular dystrophy (FSHD), Limb-girdle muscular dystrophies (LGMD), or Duchenne muscular dystrophy (DMD). In some embodiments, the bone disorder is selected from the group consisting of: osteoporosis, renal osteodystrophy, osteomalacia, osteogenesis imperfecta, fibrodysplasia ossificans progressiva, corticosteroid-induced bone loss, androgen-deprivation therapy-induced bone loss, bone fracture, cancer-induced bone loss, bone metastasis, Paget's disease of the bone, Rickets, Perthes' disease and fibrous dysplasia.

In some embodiments, the method further comprises administering a second agent to the subject in need thereof, wherein the second agent is administered prior to, concurrently with, or subsequent to administration of the pharmaceutical composition.

In another aspect, the present disclosure provides a polynucleotide encoding a protein comprising: a mutant soluble ActRIIB-ECD sequence selected from the group consisting of SEQ ID NOs: 119, 120-221, 329, 332, and 335, 336-354, and 355-372, and an Fc domain sequence of a human IgG. In some embodiments, the protein encoded by the polynucleotide further comprises: the peptide linker sequence of SEQ ID NO: 44; the hinge linker sequence of SEQ ID NO: 118; or both the peptide linker sequence of SEQ ID NO: 44 and the hinge linker sequence of SEQ ID NO: 118. In some embodiments, the polynucleotide further comprises a signal peptide sequence.

In another aspect, the present disclosure provides a polynucleotide comprising a DNA sequence of SEQ ID NO: 1653. In another aspect, the present disclosure provides a vector comprising the polynucleotide herein. In another aspect, the present disclosure provides a host cell comprising the polynucleotide herein. In some embodiments, the host cell is a mammalian cell.

In another aspect, the present disclosure provides a method of producing a protein comprising a mutant soluble ActRII-ECD comprising culturing the host cell herein under conditions promoting the expression of the protein, and recovering the protein. In some embodiments, the method further comprises purifying the protein using one or more of Protein A chromatography, size exclusion chromatography, or ion exchange chromatography.

In another aspect, the present disclosure provides a method of selecting a preferred host cell for the expression of a soluble ActRII-ECD protein, the method comprising inserting a polynucleotide encoding a soluble ActRII-ECD protein that comprises an N-linked glycosylation site at the position corresponding to N18 of SEQ ID NO: 1 into a candidate host cell, culturing the host cell under conditions promoting the expression of the protein, recovering the protein, and measuring the percentage of ActRII-ECD proteins that are aglycosylated at the N18 position. The method further comprises selecting a host cell line for production of a soluble ActRII-ECD protein if the host cell line produces ActRIIB-ECD proteins wherein at least 20%, 25%, 30%, 35%, 40%, 45%, 50%, 55%, 60%, 65%, 70%, 75%, 80%, 85%, 90% or more of the ActRII-ECD proteins in the sample are aglycosylated at the N-linked glycosylation site corresponding to position N18 of SEQ ID NO: 1. The method may further comprise measuring the percentage of ActRII-ECD proteins that are sialylated at the N-linked glycosylation site corresponding to position N41 of SEQ ID NO: 1, and selecting a host cell line that produces a relatively high percentage of sialylated ActRII proteins at that position (e.g., wherein at least 60%, 70%, 80%, 85%, 90% or more of the ActRII proteins in a sample are sialylated at the position corresponding to N41 of SEQ ID NO: 1)

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows an ActRIIB/GDF11/ALK5 complex modeled with biantennary glycan, which reveals a clash (dotted oval) in binding the ligands due to N18 glycans.

FIG. 2A shows a dimer of two fusion proteins, each comprising a soluble ActRII-ECD (200) and a Fc domain (201). Each of the fusion proteins has three N-linked glycosylation sites, at N18, N41, and N194, and O-linked glycosylation sites (202). The two fusion proteins are connected via disulfide bonds (203). Multiple exemplary sequences of fusion proteins having this structure are described herein, including but not limited to SEQ ID NO: 376. FIG. 2B shows a dimer of two fusion proteins, each comprising a mutant soluble ActRII-ECD (204) and a Fc domain (201). The mutant soluble ActRII-ECD has a N18Q mutation. The fusion protein has two N-linked glycosylation sites, at N41, and N194, and an O-linked glycosylation region (202). The two fusion proteins are connected via disulfide bonds (203). Multiple exemplary sequences of fusion proteins having this structure are described herein, including but not limited to SEQ ID NO: 222.

FIG. 3A shows myostatin (300) bound to monomeric truncated ActRIIB-ECD receptor protein (301). The N-linked glycosylation sites at N18 and N41 are distal from the binding site of myostatin on the truncated ActRIIB-ECD receptor protein. FIG. 3B shows growth differentiation factor 11 (GDF11) (302) bound to monomeric truncated ActRIIB-ECD receptor protein (301). The N-linked glycosylation sites at N18 and N41 are distal from the binding site of GDF11 on the truncated ActRIIB-ECD receptor protein.

FIG. 4 shows SDS-PAGE analysis of the fractions during purification of a fusion protein comprising an exemplary mutant soluble ActRIIB-ECD.

FIG. 5A shows ActRIIA-ECD receptor protein (orange/dark gray) aligned to ActRIIB-ECD receptor protein (cyan/light gray). The N-linked glycosylation sites at positions N18 and N41 are structurally conserved. FIG. 5B shows sequence alignment of ActRIIB-ECD with ActRIIA-ECD. The N-linked glycosylation sites at N24 and N47 (gray boxes) of the full length ActRIIA-ECD and ActRIIB-ECD correspond to positions N18 and N41, respectively, in truncated ActRIIB-ECD (SEQ ID NO: 1) and truncated ActRIIA-ECD (SEQ ID NO: 1635). The N-linked glycosylation sites at positions N18 and N41 are conserved between ActRIIA-ECD and ActRIIB-ECD. 53.8% identity is shown. The aligned sequences are: Hu ActRIIB: uniprot/Q13705 and Hu ActRIIA: uniprot/P27037.

The figures herein are for illustrative purposes only and are not necessarily drawn to scale.

### DETAILED DESCRIPTION

The present disclosure provides for isolated proteins comprising a modified activin II receptor (ActRIIA or ActRIIB) or a fragment thereof that has improved binding to a binding partner (e.g., myostatin and/or activin) compared to a reference ActRII (ActRIIA or ActRIIB) without the modification. In some aspects, the modified ActRIIA or ActRIIB may include one or more mutations that remove a glycosylation site (e.g., an N-linked glycosylation site). Removal of the glycosylation site may facilitate the binding of the ActRIIA or ActRIIB with its binding partner. The glycosylation on other glycosylation site(s) that are not removed by the mutation(s) may be retained.

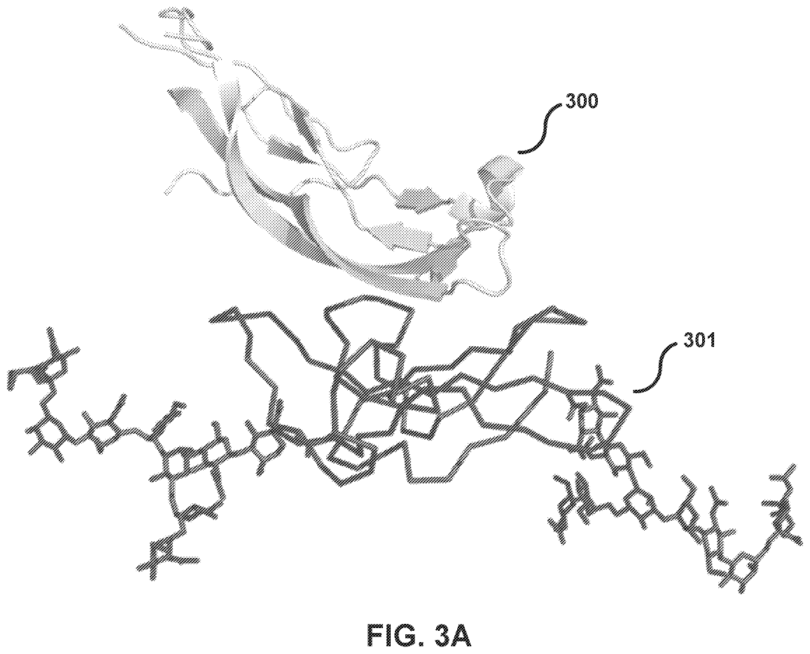

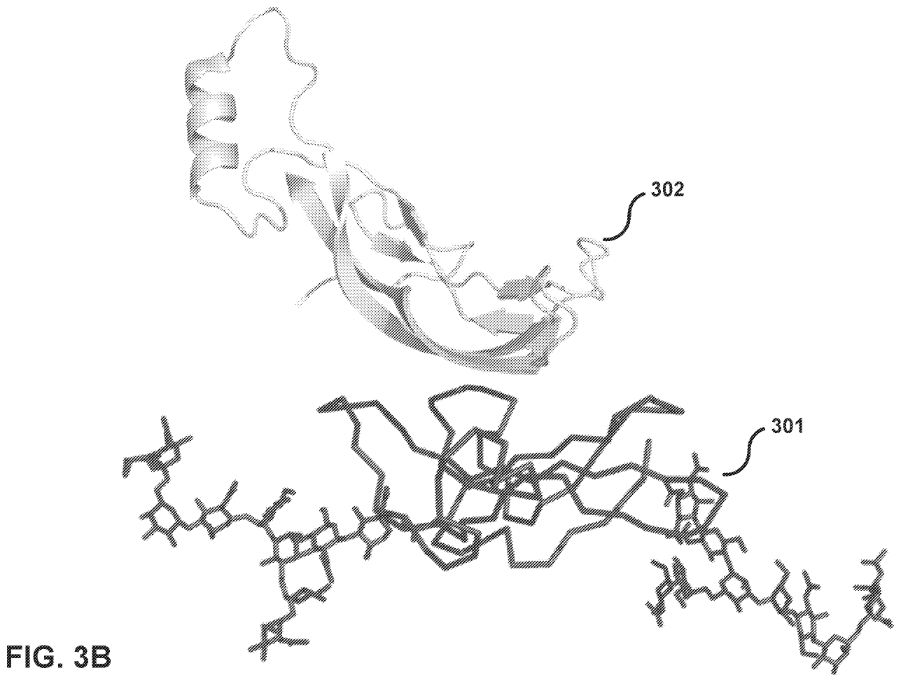

The inventors have surprisingly discovered that selective removal of a glycosylation site (e.g, the glycosylation site corresponding to position N18 of SEQ ID NO: 1) may improve binding of ActRIIB ligands (e.g., activin, myostatin, BMP9) to the ActRIIB receptor. This discovery is particularly surprising because the sites on ActRIIB for binding to its ligands (e.g., myostatin and GDF11) are distal to the glycosylation sites (see FIGS. 3A and 3B).

In some embodiments, the isolated protein comprising the modified ActRIIA or ActRIIB may exhibit better potency in treating a myostatin-related or activin A-related disorders in a subject (e.g., severe muscle loss, cachexia, and a wide range of chronic catabolic diseases that involve muscle atrophy, bone loss, inflammation, and fibrosis). In some embodiments, the isolated protein with the mutant ActRIIA or ActRIIB may have a similar or increased level of other desired features such as stability compared to the reference protein.

In one aspect, the isolated protein provided in the present disclosure may include a mutant soluble ActRIIB extracellular domain (ActRIIB-ECD). The mutant may comprise one or more mutations at the N-linked glycosylation site corresponding to position N18 of wild type human soluble ActRIIB-ECD (SEQ ID NO: 1). In some embodiments, the mutant soluble ActRIIB-ECD may comprise additional mutation(s) for functions other than the modification of glycosylation site.

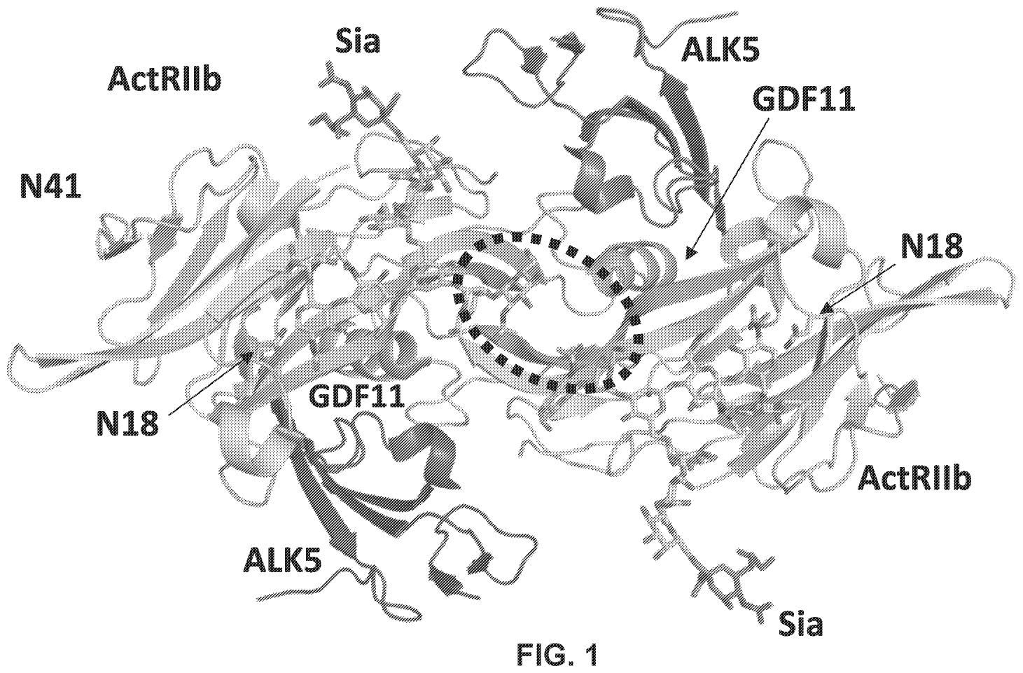

In some embodiments, the isolated protein may be a fusion protein comprising the mutant soluble ActRIIA-ECD or ActRIIB-ECD fused with a heterologous protein, e.g., an Fc domain of an immunoglobulin. The heterologous protein may be attached to the mutant soluble ActRIIA-ECD or ActRIIB-ECD via one or more linkers. In some embodiments, the isolated protein may be in the form of a dimer, e.g., through the dimerization of the heterologous protein. The inventors have surprisingly discovered that glycosylation (including sialylation) at N18 of the ActRIIB-ECD of such a dimerized fusion protein may allow only one of the two ActRIIB-ECDs to bind to ligand, i.e., monovalent binding. This may be due to steric clashing in which N18 glycans prevent binding of two copies of ligand to such a dimer (see, e.g., FIG. 1). This may lead to lower affinity for the ligand, which in turn leads to lower potency of the ActRII-ECD fusion protein. As discussed above, this discovery is particularly surprising because the ligand binding site on monomeric ActRIIB-ECD is distal to the glycosylation sites and in such a structure, the glycans do not interfere with binding of ligand (e.g., myostatin, activin, GDF11, etc.) (see FIGS. 3A and 3B). Without wishing to be bound by theory, it is believed that bivalent binding (as opposed to monovalent binding) results in higher affinity of the fusion protein for its ligand(s), which in turn leads to higher potency of the fusion protein. Accordingly, in some aspects, removal of a glycosylation site in a mutant soluble ActRII-ECD-Fc fusion protein may facilitate the binding of the ActRII with its binding partner (e.g., myostatin or activin) in the context of the dimerized fusion protein. Provided herein are compositions comprising ActRII-ECD-Fc fusion proteins that have lower (e.g., no) glycosylation (e.g., also sialylation) at the asparagine residue corresponding to position N18 of SEQ ID NO: 1 in any of the ActRII-ECD described herein or known in the art, and methods associated with such compositions.

Additionally, it is also believed that increased overall sialylation of protein therapeutics such as the modified ActRIIA-ECD or ActRIIB-ECD polypeptides described herein can lead to higher half-life of the molecule in a subject. Higher half-lives are a desirable attribute of a therapeutic. However, as described above, sialylation at the N18 position surprisingly leads to steric clashing (see, e.g., FIG. 1) that can decrease affinity/potency of ActRII molecules described herein. The inventors have surprisingly figured out a way to balance these two challenges. The modified ActRII polypeptides described herein may advantageously remove the source of steric clashing at N18 specifically (thereby increasing affinity/potency) while not interfering with the overall sialylation on the rest of the molecule (thereby preserving the half-life of the molecule). As such, provided herein are compositions that comprise a certain desired (e.g., high) level of sialylation without that sialylation being at the N18 site. Also provided herein are methods of making such compositions. For example, the compositions and methods described herein permit the tuning of sialylation to a desirable level without negatively impacting the ability of the fusion proteins to bind the desired ligands (e.g., myostatin, activin, GDF11).

Also provided herein are related compositions, kits, nucleic acids, vectors, and recombinant cells, as well as related methods, including methods of using and methods of producing any of the proteins described herein.

In another aspect, the present disclosure provides a method of selecting a preferred host cell for the expression of an ActRIIB or ActRIIA protein. The selection method comprises inserting a polynucleotide encoding an ActRII protein that comprises an N-linked glycosylation site at the position corresponding to N18 of SEQ ID NO: 1 into a candidate host cell that is capable of glycosylating proteins (e.g., a mammalian cell such as a human endothelial kidney 293 (HEK293) cell, baby hamster kidney (BHK) cell, Sp2/0 hybridoma mouse cell, Chinese hamster ovary (CHO) cell, HT-1080 human cell, or a non-mammalian cell (such as yeast) that produces glycosylated proteins). The host cell is cultured under conditions promoting the expression of the protein, the protein is recovered, and the percentage of ActRII-ECD proteins that are aglycosylated at the N18 position is measured. The host cell line is selected for production of a soluble ActRII-ECD protein if at least 20%, 25%, 30%, 35%, 40%, 45%, 50%, 55%, 60%, 65%, 70%, 75%, 80%, 85%, 90% or more of the ActRII-ECD proteins are aglycosylated at the N-linked glycosylation site corresponding to position N18 of SEQ ID NO: 1. The method may further comprise selecting a host cell line for production of an ActRII protein based on further criteria in addition to aglycosylation at the N18 position, including preferably selecting a host cell line that additionally produces a relatively high percentage of sialylated ActRII proteins at the position corresponding to N41 of SEQ ID NO: 1 (e.g., wherein at least 60%, 70%, 80%, 85%, 90% or more of the ActRII proteins in a sample are sialylated at the position corresponding to N41 of SEQ ID NO: 1).

### Definitions

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free a-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
1) Alanine (A), Serine(S), and Threonine (T)
2) Aspartic acid (D) and Glutamic acid (E)
3) Asparagine (N) and Glutamine (Q)
4) Arginine (R) and Lysine (K)
5) Isoleucine (I), Leucine (L), Methionine (M), and Valine (V)
6) Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within ±2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−. 1); glutamate (+3.0.+−. 1); serine (+0.3); asparagine (+0.2); glutamine (+0.2); glycine (0); threonine (−0.4); proline (−0.5.+−. 1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within #2 is included, in various embodiments, those that are within #1 are included, and in various embodiments, those within #0.5 are included.

Exemplary amino acid substitutions are set forth in Table 1.

TABLE 1

| Original Residues | Exemplary Substitutions | Preferred Substitutions |
|---|---|---|
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of polypeptides as set forth herein using well-known techniques. In various embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In various embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The terms “polypeptide fragment” and “truncated polypeptide” as used herein refer to a polypeptide that has an amino-terminal and/or carboxy-terminal deletion as compared to a corresponding full-length protein. In certain embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In certain embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant” and “polypeptide mutant” as used herein refer to a polypeptide that comprises an amino acid sequence wherein one or more amino acid residues are inserted into, deleted from and/or substituted into the amino acid sequence relative to another polypeptide sequence. In certain embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Hybrids of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. In certain embodiments, the % identity is selected from, e.g., at least 60%, at least 65%, at least 70%, at least 75%, at least 80%, at least 85%, at least 90%, at least 95%, or at least 99% or more sequence identity to a given sequence. In certain embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In certain embodiments, the % homology is selected from, e.g., at least 60%, at least 65%, at least 70%, at least 75%, at least 80%, at least 85%, at least 90%, at least 95%, or at least 99% or more sequence homology to a given sequence. In certain embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See Id.

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “antibody” as used herein refers to a protein comprising one or more polypeptides substantially or partially encoded by immunoglobulin genes or fragments of immunoglobulin genes and having specificity to a tumor antigen or specificity to a molecule overexpressed in a pathological state. The recognized immunoglobulin genes include the kappa, lambda, alpha, gamma, delta, epsilon and mu constant region genes, as well as subtypes of these genes and myriad of immunoglobulin variable region genes. Light chains (LC) are classified as either kappa or lambda. Heavy chains (HC) are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition.

The term “Fc region” or “Fc domain” as used herein defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a C_{H2} domain and a C_{H3} domain, and optionally comprises a C_{H4} domain. The Fc portion of an antibody mediates several important effector functions e.g. cytokine induction, ADCC, phagocytosis, complement dependent cytotoxicity (CDC) and half-life/clearance rate of antibody and antigen-antibody complexes (e.g., the neonatal FcR (FcRn) binds to the Fc region of IgG at acidic pH in the endosome and protects IgG from degradation, thereby contributing to the long serum half-life of IgG). Replacements of amino acid residues in the Fc portion to alter antibody effector function are known in the art (see, e.g., Winter et al., U.S. Pat. Nos. 5,648,260 and 5,624,821).

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions.

“Probe,” when used in reference to a polynucleotide, refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a protein or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The terms “label” or “labeled” as used herein refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin β, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

The terms “treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

It is understood that aspect and embodiments of the disclosure described herein include “consisting” and/or “consisting essentially of” aspects and embodiments.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variations.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. Reference to “about” a value or parameter herein includes (and describes) variations that are directed to that value or parameter per se. For example, description referring to “about X” includes description of “X”.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

When a term refers to a protein, the term encompasses both the full-length of the protein as well as a functional fragment of the protein. The term “functional fragment” means that the sequence of the polypeptide may include less amino-acid than the original sequence but still enough amino-acids to confer the enzymatic activity of the original sequence of reference. It is well known in the art that a polypeptide can be modified by substitution, insertion, deletion and/or addition of one or more amino-acids while retaining its enzymatic activity. For example, substitutions of one amino-acid at a given position by chemically equivalent amino-acids that do not affect the functional properties of a protein are common.

### Activin II Receptors

In one aspect, the present disclosure provides an isolated protein comprising a mutant ActRIIA or ActRIIB polypeptide. The mutant activin type II B receptors (ActRIIB) refers to a mutant of the human activin receptors having accession number NP_001097.2 (SEQ ID NO: 45), and variants thereof. The mutant ActRIIA refers to a mutant of the human activin receptors having a sequence of SEQ ID NO: 47, and variants thereof.

In some embodiments, the isolated protein according to the present disclosure may comprise a mutant ActRIIB extracellular domain (ActRIIB-ECD), which is a mutant of the wild-type ActRIIB-ECD. The wild-type ActRIIB-ECD refers to the extracellular domain of ActRIIB, amino acids 1 to 134 (with signal sequence), or amino acids 19 through 134 of SEQ ID NO: 45 (without signal sequence) (referred to herein as SEQ ID NO: 46). The term wild-type ActRIIB-ECD may also refer to a functional, truncated form of the ActRIIB-ECD, for example the truncated sequence of SEQ ID NO: 1.

In some embodiments, the isolated protein according to the present disclosure may comprise a mutant of the wild-type soluble ActRIIB-ECD polypeptide. The wild type soluble ActRIIB-ECD polypeptide may be a truncated form of ActRIIB-ECD. In some examples, the wild type soluble ActRIIB-ECD has a sequence of SEQ ID NO: 1.

In some embodiments, the isolated protein according to the present disclosure may comprise a mutant of the wild-type soluble ActRIIA-ECD polypeptide. The wild type soluble ActRIIA-ECD polypeptide may be a truncated form of ActRIIA-ECD. In some examples, the wild type soluble ActRIIB-ECD has a sequence of SEQ ID NO: 1654.

Mutation(s) that Remove Glycosylation Site

The mutant soluble ActRII-ECD polypeptide may comprise one or more mutations that remove a glycosylation site in the wild type soluble ActRII-ECD. In some embodiments, the glycosylation site may be an N-linked glycosylation site. The N-linked glycosylation site may be a site corresponding to position N18 of SEQ ID NO: 1. Unless otherwise stated, references herein to an amino acid position of an ActRII-ECD polypeptide (e.g., position “N18” or “N41” of an ActRII-ECD polypeptide) refer to the corresponding positions as numbered in SEQ ID NO: 1. Without wishing to be bound by theory, removal of the N18 glycosylation site may reduce steric hindrance in the ligand binding site of the ActRII protein. For example, FIG. 1 shows a ActRIIB/GDF11/ALK5 complex modeled with biantennary glycan, which reveals a clash in binding two copies of ligand due to N18 glycans. Accordingly, the inventors envision that the removal of the N18 glycosylation site from various soluble ActRII-ECD polypeptides will result in similar improvements to ligand binding affinity.

In some embodiments, the mutant soluble ActRII-ECD may comprise a mutation at position corresponding to position N18 of SEQ ID NO: 1 (i.e., the asparagine at position N18 is substituted with another amino acid). For example, the mutant soluble ActRIIB-ECD may comprise a sequence of SEQ ID NO: 223. In another example, the mutant soluble ActRIIB-ECD may consist of a sequence of SEQ ID NO: 223. In some examples, the mutant soluble ActRIIB-ECD may comprise a N18Q mutation, i.e., in the mutant soluble ActRIIB-ECD, the asparagine at position N18 or a position corresponding to N18 is substituted with glutamine. For example, the mutant soluble ActRIIB-ECD may comprise a sequence of SEQ ID NO: 119. In another example, the mutant soluble ActRIIB-ECD may consist of a sequence of SEQ ID NO: 119. In one example, the mutant soluble ActRIIB-ECD may comprise a sequence of SEQ ID NO: 146. In another example, the mutant soluble ActRIIB-ECD may consist of a sequence of SEQ ID NO: 146. In another example, the mutant soluble ActRIIB-ECD may comprise a sequence of SEQ ID NO: 336. In another example, the mutant soluble ActRIIB-ECD may consist of a sequence of SEQ ID NO: 336. In another example, the mutant soluble ActRIIB-ECD may comprise a sequence of SEQ ID NO: 337. In another example, the mutant soluble ActRIIB-ECD may consist of a sequence of SEQ ID NO: 337. In another example, the mutant soluble ActRIIB-ECD may comprise a sequence of SEQ ID NO: 338. In another example, the mutant soluble ActRIIB-ECD may consist of a sequence of SEQ ID NO: 338. In another example, the mutant soluble ActRIIB-ECD may comprise a sequence of SEQ ID NO: 339. In another example, the mutant soluble ActRIIB-ECD may consist of a sequence of SEQ ID NO: 339. In another example, the mutant soluble ActRIIB-ECD may comprise a sequence of SEQ ID NO: 340. In another example, the mutant soluble ActRIIB-ECD may consist of a sequence of SEQ ID NO: 340. In another example, the mutant soluble ActRIIB-ECD may comprise a sequence of SEQ ID NO: 341. In another example, the mutant soluble ActRIIB-ECD may consist of a sequence of SEQ ID NO: 341. In another example, the mutant soluble ActRIIB-ECD may comprise a sequence of SEQ ID NO: 342. In another example, the mutant soluble ActRIIB-ECD may consist of a sequence of SEQ ID NO: 342. In another example, the mutant soluble ActRIIB-ECD may comprise a sequence of SEQ ID NO: 343. In another example, the mutant soluble ActRIIB-ECD may consist of a sequence of SEQ ID NO: 343. In another example, the mutant soluble ActRIIB-ECD may comprise a sequence of SEQ ID NO: 344. In another example, the mutant soluble ActRIIB-ECD may consist of a sequence of SEQ ID NO: 344. In another example, the mutant soluble ActRIIB-ECD may comprise a sequence of SEQ ID NO: 345. In another example, the mutant soluble ActRIIB-ECD may consist of a sequence of SEQ ID NO: 345. In another example, the mutant soluble ActRIIB-ECD may comprise a sequence of SEQ ID NO: 346. In another example, the mutant soluble ActRIIB-ECD may consist of a sequence of SEQ ID NO: 346. In another example, the mutant soluble ActRIIB-ECD may comprise a sequence of SEQ ID NO: 347. In another example, the mutant soluble ActRIIB-ECD may consist of a sequence of SEQ ID NO: 347. In another example, the mutant soluble ActRIIB-ECD may comprise a sequence of SEQ ID NO: 348. In another example, the mutant soluble ActRIIB-ECD may consist of a sequence of SEQ ID NO: 348. In another example, the mutant soluble ActRIIB-ECD may comprise a sequence of SEQ ID NO: 349. In another example, the mutant soluble ActRIIB-ECD may consist of a sequence of SEQ ID NO: 349. In another example, the mutant soluble ActRIIB-ECD may comprise a sequence of SEQ ID NO: 350. In another example, the mutant soluble ActRIIB-ECD may consist of a sequence of SEQ ID NO: 350. In another example, the mutant soluble ActRIIB-ECD may comprise a sequence of SEQ ID NO: 351. In another example, the mutant soluble ActRIIB-ECD may consist of a sequence of SEQ ID NO: 351. In another example, the mutant soluble ActRIIB-ECD may comprise a sequence of SEQ ID NO: 352. In another example, the mutant soluble ActRIIB-ECD may consist of a sequence of SEQ ID NO: 352. In another example, the mutant soluble ActRIIB-ECD may comprise a sequence of SEQ ID NO: 353. In another example, the mutant soluble ActRIIB-ECD may consist of a sequence of SEQ ID NO: 353. In another example, the mutant soluble ActRIIB-ECD may comprise a sequence of SEQ ID NO: 354. In another example, the mutant soluble ActRIIB-ECD may consist of a sequence of SEQ ID NO: 354.

In some embodiments, the isolated protein may comprise a mutant ActRIIB-ECD, which may comprise a sequence selected from the group consisting of SEQ ID NOs: 1, 119, 146, 223, or 336-354, and the mutant ActRIIB-ECD may further comprise from 1 to 6 additional amino acids on the N-terminus of the sequence corresponding to amino acids 1-6 of SEQ ID NO: 46. In some examples, the mutant ActRIIB-ECD may comprise a sequence of SEQ ID NO: 377, in which N19 is substituted with another amino acid. For example, the mutant ActRIIB-ECD may comprise a sequence of SEQ ID NO: 382. In some examples, the mutant ActRIIB-ECD may comprise a sequence of SEQ ID NO: 378, in which N20 is substituted with another amino acid. For example, the mutant ActRIIB-ECD may comprise a sequence of SEQ ID NO: 383. In some examples, the mutant ActRIIB-ECD may comprise a sequence of SEQ ID NO: 379, in which N21 is substituted with another amino acid. For example, the mutant ActRIIB-ECD may comprise a sequence of SEQ ID NO: 384. In some examples, the mutant ActRIIB-ECD may comprise a sequence of SEQ ID NO: 380, in which N22 is substituted with another amino acid. For example, the mutant ActRIIB-ECD may comprise a sequence of SEQ ID NO: 385. In some examples, the mutant ActRIIB-ECD may comprise a sequence of SEQ ID NO: 381, in which N23 is substituted with another amino acid. For example, the mutant ActRIIB-ECD may comprise a sequence of SEQ ID NO: 386. In some examples, the mutant ActRIIB-ECD may comprise a sequence of SEQ ID NO: 46, in which N24 is substituted with another amino acid. For example, the mutant ActRIIB-ECD may comprise a sequence of SEQ ID NO: 387.

In some examples, the mutant ActRIIB-ECD may comprise a sequence of SEQ ID NO: 377, in which S21 is substituted with another amino acid other than serine(S) or threonine (T). In some examples, the mutant ActRIIB-ECD may comprise a sequence of SEQ ID NO: 378, in which S22 is substituted with another amino acid other than serine(S) or threonine (T). In some examples, the mutant ActRIIB-ECD may comprise a sequence of SEQ ID NO: 379, in which S23 is substituted with another amino acid other than serine(S) or threonine (T). In some examples, the mutant ActRIIB-ECD may comprise a sequence of SEQ ID NO: 380, in which S24 is substituted with another amino acid other than serine(S) or threonine (T). In some examples, the mutant ActRIIB-ECD may comprise a sequence of SEQ ID NO: 381, in which S25 is substituted with another amino acid other than serine(S) or threonine (T). In some examples, the mutant ActRIIB-ECD may comprise a sequence of SEQ ID NO: 46, in which S26 is substituted with another amino acid other than serine(S) or threonine (T).

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with another amino acid. For example, the mutant soluble ActRIIB-ECD polypeptide may comprise a sequence at least 95% identical to SEQ ID NO: 1, in which the asparagine at position N18 is substituted with another amino acid.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with glutamine. For example, the mutant soluble ActRIIB-ECD polypeptide may comprise a sequence at least 95% identical to SEQ ID NO: 1, in which the asparagine at position N18 is substituted with glutamine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with alanine. For example, the mutant soluble ActRIIB-ECD polypeptide may comprise a sequence at least 95% identical to SEQ ID NO: 1, in which the asparagine at position N18 is substituted with alanine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with arginine. For example, the mutant soluble ActRIIB-ECD polypeptide may comprise a sequence at least 95% identical to SEQ ID NO: 1, in which the asparagine at position N18 is substituted with arginine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with aspartate. For example, the mutant soluble ActRIIB-ECD polypeptide may comprise a sequence at least 95% identical to SEQ ID NO: 1, in which the asparagine at position N18 is substituted with aspartate.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with cysteine. For example, the mutant soluble ActRIIB-ECD polypeptide may comprise a sequence at least 95% identical to SEQ ID NO: 1, in which the asparagine at position N18 is substituted with cysteine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with glutamate. For example, the mutant soluble ActRIIB-ECD polypeptide may comprise a sequence at least 95% identical to SEQ ID NO: 1, in which the asparagine at position N18 is substituted with glutamate.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with glycine. For example, the mutant soluble ActRIIB-ECD polypeptide may comprise a sequence at least 95% identical to SEQ ID NO: 1, in which the asparagine at position N18 is substituted with glycine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with histidine. For example, the mutant soluble ActRIIB-ECD polypeptide may comprise a sequence at least 95% identical to SEQ ID NO: 1, in which the asparagine at position N18 is substituted with histidine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with isoleucine. For example, the mutant soluble ActRIIB-ECD polypeptide may comprise a sequence at least 95% identical to SEQ ID NO: 1, in which the asparagine at position N18 is substituted with isoleucine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with leucine. For example, the mutant soluble ActRIIB-ECD polypeptide may comprise a sequence at least 95% identical to SEQ ID NO: 1, in which the asparagine at position N18 is substituted with leucine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with lysine. For example, the mutant soluble ActRIIB-ECD polypeptide may comprise a sequence at least 95% identical to SEQ ID NO: 1, in which the asparagine at position N18 is substituted with lysine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with methionine. For example, the mutant soluble ActRIIB-ECD polypeptide may comprise a sequence at least 95% identical to SEQ ID NO: 1, in which the asparagine at position N18 is substituted with methionine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with phenylalanine. For example, the mutant soluble ActRIIB-ECD polypeptide may comprise a sequence at least 95% identical to SEQ ID NO: 1, in which the asparagine at position N18 is substituted with phenylalanine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with proline. For example, the mutant soluble ActRIIB-ECD polypeptide may comprise a sequence at least 95% identical to SEQ ID NO: 1, in which the asparagine at position N18 is substituted with proline.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with serine. For example, the mutant soluble ActRIIB-ECD polypeptide may comprise a sequence at least 95% identical to SEQ ID NO: 1, in which the asparagine at position N18 is substituted with serine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with threonine. For example, the mutant soluble ActRIIB-ECD polypeptide may comprise a sequence at least 95% identical to SEQ ID NO: 1, in which the asparagine at position N18 is substituted with threonine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with tryptophan. For example, the mutant soluble ActRIIB-ECD polypeptide may comprise a sequence at least 95% identical to SEQ ID NO: 1, in which the asparagine at position N18 is substituted with tryptophan.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with tyrosine. For example, the mutant soluble ActRIIB-ECD polypeptide may comprise a sequence at least 95% identical to SEQ ID NO: 1, in which the asparagine at position N18 is substituted with tyrosine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the asparagine at position N18 is substituted with valine. For example, the mutant soluble ActRIIB-ECD polypeptide may comprise a sequence at least 95% identical to SEQ ID NO: 1, in which the asparagine at position N18 is substituted with valine.

In some embodiments, the mutation on the N-linked glycosylation site may be on an amino acid residue corresponding to position S20 of SEQ ID NO: 1. In some examples, in the mutant soluble ActRIIB-ECD, the serine at position corresponding to S20 of SEQ ID NO: 1 may be substituted with an amino acid residue that is not serine or threonine. For example, the mutant soluble ActRIIB-ECD may comprise a sequence of SEQ ID NO: 326. For example, the mutant soluble ActRIIB-ECD may comprise a sequence of SEQ ID NO: 355. In another example, the mutant soluble ActRIIB-ECD may consist of a sequence of SEQ ID NO: 355. In another example, the mutant soluble ActRIIB-ECD may comprise a sequence of SEQ ID NO: 356. In another example, the mutant soluble ActRIIB-ECD may consist of a sequence of SEQ ID NO: 356. In another example, the mutant soluble ActRIIB-ECD may comprise a sequence of SEQ ID NO: 357. In another example, the mutant soluble ActRIIB-ECD may consist of a sequence of SEQ ID NO: 357. In another example, the mutant soluble ActRIIB-ECD may comprise a sequence of SEQ ID NO: 358. In another example, the mutant soluble ActRIIB-ECD may consist of a sequence of SEQ ID NO: 358. In another example, the mutant soluble ActRIIB-ECD may comprise a sequence of SEQ ID NO: 359. In another example, the mutant soluble ActRIIB-ECD may consist of a sequence of SEQ ID NO: 359. In another example, the mutant soluble ActRIIB-ECD may comprise a sequence of SEQ ID NO: 360. In another example, the mutant soluble ActRIIB-ECD may consist of a sequence of SEQ ID NO: 360. In another example, the mutant soluble ActRIIB-ECD may comprise a sequence of SEQ ID NO: 361. In another example, the mutant soluble ActRIIB-ECD may consist of a sequence of SEQ ID NO: 361. In another example, the mutant soluble ActRIIB-ECD may comprise a sequence of SEQ ID NO: 362. In another example, the mutant soluble ActRIIB-ECD may consist of a sequence of SEQ ID NO: 362. In another example, the mutant soluble ActRIIB-ECD may comprise a sequence of SEQ ID NO: 363. In another example, the mutant soluble ActRIIB-ECD may consist of a sequence of SEQ ID NO: 363. In another example, the mutant soluble ActRIIB-ECD may comprise a sequence of SEQ ID NO: 364. In another example, the mutant soluble ActRIIB-ECD may consist of a sequence of SEQ ID NO: 364. In another example, the mutant soluble ActRIIB-ECD may comprise a sequence of SEQ ID NO: 365. In another example, the mutant soluble ActRIIB-ECD may consist of a sequence of SEQ ID NO: 365. In another example, the mutant soluble ActRIIB-ECD may comprise a sequence of SEQ ID NO: 366. In another example, the mutant soluble ActRIIB-ECD may consist of a sequence of SEQ ID NO: 366. In another example, the mutant soluble ActRIIB-ECD may comprise a sequence of SEQ ID NO: 367. In another example, the mutant soluble ActRIIB-ECD may consist of a sequence of SEQ ID NO: 367. In another example, the mutant soluble ActRIIB-ECD may comprise a sequence of SEQ ID NO: 368. In another example, the mutant soluble ActRIIB-ECD may consist of a sequence of SEQ ID NO: 368. In another example, the mutant soluble ActRIIB-ECD may comprise a sequence of SEQ ID NO: 369. In another example, the mutant soluble ActRIIB-ECD may consist of a sequence of SEQ ID NO: 369. In another example, the mutant soluble ActRIIB-ECD may comprise a sequence of SEQ ID NO: 370. In another example, the mutant soluble ActRIIB-ECD may consist of a sequence of SEQ ID NO: 370. In another example, the mutant soluble ActRIIB-ECD may comprise a sequence of SEQ ID NO: 371. In another example, the mutant soluble ActRIIB-ECD may consist of a sequence of SEQ ID NO: 371. In another example, the mutant soluble ActRIIB-ECD may comprise a sequence of SEQ ID NO: 372. In another example, the mutant soluble ActRIIB-ECD may consist of a sequence of SEQ ID NO: 372. In some embodiments, the isolated protein may comprise a mutant ActRIIB-ECD, which may comprise a sequence selected from the group consisting of SEQ ID NOs: 355-372, wherein the mutant ActRIIB-ECD may further comprise from 1 to 6 additional amino acids on the N-terminus of the sequence corresponding to amino acids 1-6 of SEQ ID NO: 46.

In some example, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is be substituted with an amino acid residue that is not serine or threonine. For example, the mutant soluble ActRIIB-ECD polypeptide may comprise a sequence at least 95% identical to SEQ ID NO: 1, in which the serine at position S20 is substituted with an amino acid residue that is not serine(S) or threonine (T).

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with alanine. For example, the mutant soluble ActRIIB-ECD polypeptide may comprise a sequence at least 95% identical to SEQ ID NO: 1, in which the serine at position S20 is substituted with alanine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with arginine. For example, the mutant soluble ActRIIB-ECD polypeptide may comprise a sequence at least 95% identical to SEQ ID NO: 1, in which the serine at position S20 is substituted with arginine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with asparagine. For example, the mutant soluble ActRIIB-ECD polypeptide may comprise a sequence at least 95% identical to SEQ ID NO: 1, in which the serine at position S20 is substituted with asparagine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with aspartate. For example, the mutant soluble ActRIIB-ECD polypeptide may comprise a sequence at least 95% identical to SEQ ID NO: 1, in which the serine at position S20 is substituted with aspartate.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with cysteine. For example, the mutant soluble ActRIIB-ECD polypeptide may comprise a sequence at least 95% identical to SEQ ID NO: 1, in which the serine at position S20 is substituted with cysteine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with glutamate. For example, the mutant soluble ActRIIB-ECD polypeptide may comprise a sequence at least 95% identical to SEQ ID NO: 1, in which the serine at position S20 is substituted with glutamate.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with glutamine. For example, the mutant soluble ActRIIB-ECD polypeptide may comprise a sequence at least 95% identical to SEQ ID NO: 1, in which the serine at position S20 is substituted with glutamine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with glycine. For example, the mutant soluble ActRIIB-ECD polypeptide may comprise a sequence at least 95% identical to SEQ ID NO: 1, in which the serine at position S20 is substituted with glycine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with histidine. For example, the mutant soluble ActRIIB-ECD polypeptide may comprise a sequence at least 95% identical to SEQ ID NO: 1, in which the serine at position S20 is substituted with histidine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with isoleucine. For example, the mutant soluble ActRIIB-ECD polypeptide may comprise a sequence at least 95% identical to SEQ ID NO: 1, in which the serine at position S20 is substituted with isoleucine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with leucine. For example, the mutant soluble ActRIIB-ECD polypeptide may comprise a sequence at least 95% identical to SEQ ID NO: 1, in which the serine at position S20 is substituted with leucine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with lysine. For example, the mutant soluble ActRIIB-ECD polypeptide may comprise a sequence at least 95% identical to SEQ ID NO: 1, in which the serine at position S20 is substituted with lysine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with methionine. For example, the mutant soluble ActRIIB-ECD polypeptide may comprise a sequence at least 95% identical to SEQ ID NO: 1, in which the serine at position S20 is substituted with methionine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with phenylalanine. For example, the mutant soluble ActRIIB-ECD polypeptide may comprise a sequence at least 95% identical to SEQ ID NO: 1, in which the serine at position S20 is substituted with phenylalanine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with proline. For example, the mutant soluble ActRIIB-ECD polypeptide may comprise a sequence at least 95% identical to SEQ ID NO: 1, in which the serine at position S20 is substituted with proline.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with tryptophan. For example, the mutant soluble ActRIIB-ECD polypeptide may comprise a sequence at least 95% identical to SEQ ID NO: 1, in which the serine at position S20 is substituted with tryptophan.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with tyrosine. For example, the mutant soluble ActRIIB-ECD polypeptide may comprise a sequence at least 95% identical to SEQ ID NO: 1, in which the serine at position S20 is substituted with tyrosine.

In some examples, the mutant soluble ActRIIB-ECD polypeptide may comprise a sequence at least 80% (e.g., at least 85%, 90%, 95%, 96%, 97%, or 98%) identical to SEQ ID NO: 1, in which the serine at position S20 is substituted with valine. For example, the mutant soluble ActRIIB-ECD polypeptide may comprise a sequence at least 95% identical to SEQ ID NO: 1, in which the serine at position S20 is substituted with valine.

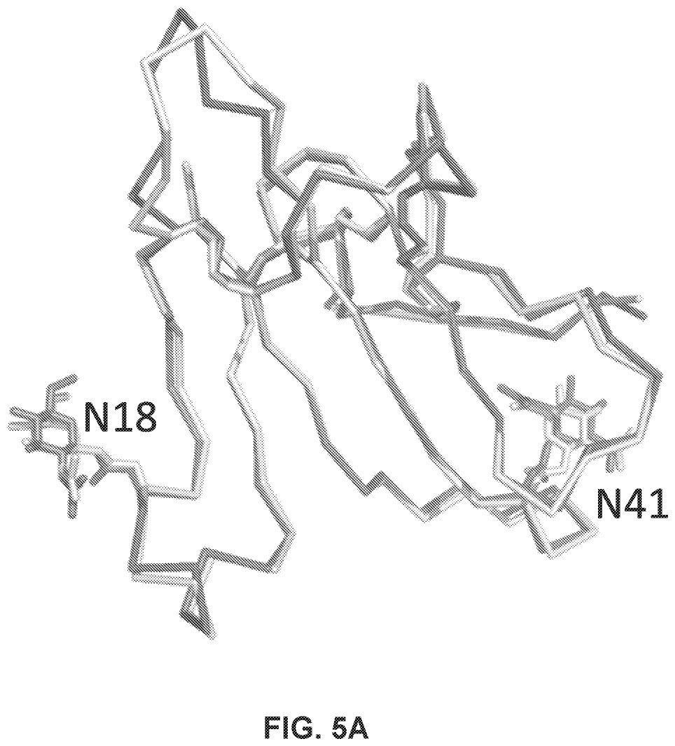

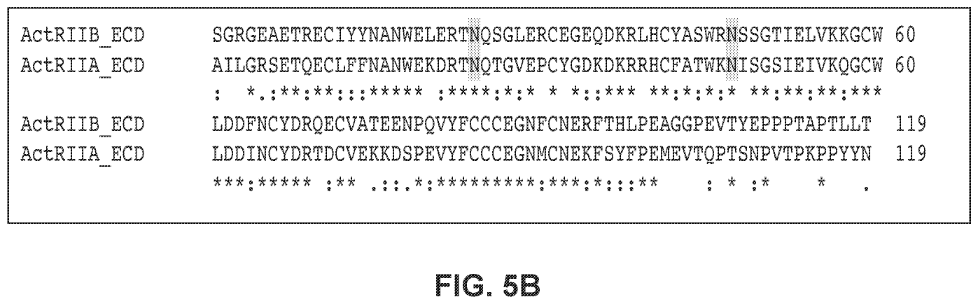

In some aspects, the isolated protein may comprise a mutant soluble ActRIIA-ECD. The mutant soluble ActRIIA-ECD may comprise any mutations corresponding to any of the mutations in the mutant soluble ActRIIB-ECD described herein, or other mutations to ActRIIA-ECD known in the art. The N18 and N41 glycosylation sites are conserved between ActRIIA-ECD and ActRIIB-ECD (see FIGS. 5A and 5B). Thus, N18 of soluble ActRIIA-ECD corresponds to N18 of soluble ActRIIB-ECD. T20 of soluble ActRIIA-ECD corresponds to S20 of soluble ActRIIB-ECD. N41 of soluble ActRIIA-ECD corresponds to N41 of soluble ActRIIB-ECD. In some embodiments, the isolated protein may comprise a mutant soluble ActRIIA-ECD comprising a mutation that removes the glycosylation site at N18 of SEQ ID NO: 1654. SEQ ID NO: 1654 is a truncated form of wild-type human ActRIIA-ECD (SEQ ID NO: 48), in which the first six amino acids at the N-terminus of SEQ ID NO: 48 are truncated.

In some embodiments, the mutant soluble ActRIIA-ECD may comprise a mutation at position corresponding to position N18 of SEQ ID NO: 1654 (i.e., the asparagine at position N18 is substituted with another amino acid). For example, the mutant soluble ActRIIA-ECD may comprise a sequence of SEQ ID NO: 1655.

In some examples, the mutant soluble ActRIIA-ECD may comprise a N18Q mutation, i.e., in the mutant soluble ActRIIA-ECD, the asparagine at position N18 or a position corresponding to N18 is substituted with glutamine. For example, the mutant soluble ActRIIA-ECD may comprise a sequence of SEQ ID NO: 1656. In another example, the mutant soluble ActRIIA-ECD may consist of a sequence of SEQ ID NO: 1656.

In some examples, the mutant soluble ActRIIA-ECD polypeptide may comprise a sequence at least 80% (e.g., at least 85%, 90%, 95%, 96%, 97%, or 98%) identical to SEQ ID NO: 1655, in which the asparagine at position N18 is substituted with another amino acid. For example, the mutant soluble ActRIIA-ECD polypeptide may comprise a sequence at least 95% identical to SEQ ID NO: 1655, in which the asparagine at position N18 is substituted with another amino acid.

In some examples, the mutant soluble ActRIIA-ECD polypeptide may comprise a sequence at least 80% (e.g., at least 85%, 90%, 95%, 96%, 97%, or 98%) identical to SEQ ID NO: 1656, in which the asparagine at position N18 is substituted with glutamine. For example, the mutant soluble ActRIIA-ECD polypeptide may comprise a sequence at least 95% identical to SEQ ID NO: 1656, in which the asparagine at position N18 is substituted with glutamine.

In some embodiments, the mutant soluble ActRIIA-ECD may comprise a mutation at position corresponding to position T20 of SEQ ID NO: 1654 (i.e., the asparagine at position T20 is substituted with another amino acid other than serine(S) or threonine (T)). For example, the mutant soluble ActRIIA-ECD may comprise a sequence of SEQ ID NO: 1657.

In some examples, the mutant soluble ActRIIA-ECD polypeptide may comprise a sequence at least 80% (e.g., at least 85%, 90%, 95%, 96%, 97%, or 98%) identical to SEQ ID NO: 1657, in which the asparagine at position T20 is substituted with another amino acid than serine(S) or threonine (T). For example, the mutant soluble ActRIIA-ECD polypeptide may comprise a sequence at least 95% identical to SEQ ID NO: 1655, in which the asparagine at position T20 is substituted with another amino acid than serine(S) or threonine (T).

In some embodiments, the isolated protein may comprise a mutant soluble ActRIIA-ECD, which may comprise a sequence selected from the group consisting of SEQ ID NOs: 1655, 1656, and 1657, and the mutant soluble ActRIIA-ECD may further comprise from 1 to 6 additional amino acids on the N-terminus of the sequence corresponding to amino acids 1-6 of SEQ ID NO: 48. In one example, the mutant soluble ActRIIA-ECD may have comprise S on the N-terminus. In one example, the mutant soluble ActRIIA-ECD may have comprise RS on the N-terminus. In one example, the mutant soluble ActRIIA-ECD may have comprise GRS on the N-terminus. In one example, the mutant soluble ActRIIA-ECD may have comprise LGRS on the N-terminus. In one example, the mutant soluble ActRIIA-ECD may have comprise ILGRS on the N-terminus. In one example, the mutant soluble ActRIIA-ECD may have comprise AILGRS on the N-terminus.

### Additional Mutation(s)

Besides the one or more mutations that remove a glycosylation site (e.g., the N-linked glycosylation site), the mutant soluble ActRIIB-ECD polypeptide may further comprise one or more additional mutations.

In some embodiments, the one or more additional mutations may be introduced so that the mutant soluble ActRIIB-ECD demonstrates a marked reduction of BMP9-neutralization as compared to a wild-type soluble ActRIIB-ECD or mutant soluble ActRIIB-ECD without the additional mutations, while retaining (e.g., fully retaining) myostatin- and activin A-neutralization. In some embodiments, the additional mutations may be introduced by replacing one or more amino acids of a wild-type soluble ActRIIB-ECD (SEQ ID NO: 1) with the amino acids from a wild-type soluble ActRIIA-ECD (SEQ ID NO: 2) at corresponding position(s) based on sequence alignment between the two soluble ActRII ECD domains at the amino acid level. The term “wild-type ActRIIA-ECD” refers to the extracellular domain of ActRIIA, amino acids 1 to 135 (with signal sequence), or amino acids 20 through 135 of SEQ ID NO: 47 (without signal sequence) (referred to herein as SEQ ID NO: 48).

In some embodiments, the one or more additional mutations may be introduced so that at least one of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least two of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least three of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least four of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least five of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least six of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least seven of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least eight of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least nine of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least ten of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least fifteen of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least twenty of amino acid residues corresponding to R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least twenty-five of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2). In some embodiments, the one or more additional mutations may be introduced so that at least thirty of amino acid residues corresponding to R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 is substituted with another amino acid residue (e.g., amino acid residue in the corresponding position of SEQ ID NO: 2).

In some embodiments, the mutant soluble ActRIIB-ECD may comprise a sequence of any one of SEQ ID NOs: 3-37 and 51-117, in which the asparagine at position N18 is substituted with another amino acid. In some examples, the mutant soluble ActRIIB-ECD may comprise a sequence of SEQ ID NO: 224. In some examples, the mutant soluble ActRIIB-ECD may comprise a sequence of SEQ ID NO: 225. In some examples, the mutant soluble ActRIIB-ECD may comprise a sequence of SEQ ID NO: 226. In some examples, the mutant soluble ActRIIB-ECD may comprise a sequence of SEQ ID NO: 227. In some examples, the mutant soluble ActRIIB-ECD may comprise a sequence of SEQ ID NO: 228. In some examples, the mutant soluble ActRIIB-ECD may comprise a sequence of SEQ ID NO: 229. In some examples, the mutant soluble ActRIIB-ECD may comprise a sequence of SEQ ID NO: 230. In some examples, the mutant soluble ActRIIB-ECD may comprise a sequence of SEQ ID NO: 231. In some examples, the mutant soluble ActRIIB-ECD may comprise a sequence of SEQ ID NO: 232. In some examples, the mutant soluble ActRIIB-ECD may comprise a sequence of SEQ ID NO: 233. In some examples, the mutant soluble ActRIIB-ECD may comprise a sequence of SEQ ID NO: 234. In some examples, the mutant soluble ActRIIB-ECD may comprise a sequence of SEQ ID NO: 235. In some examples, the mutant soluble ActRIIB-ECD may comprise a sequence of SEQ ID NO: 236. In some examples, the mutant soluble ActRIIB-ECD may comprise a sequence of SEQ ID NO: 237. In some examples, the mutant soluble ActRIIB-ECD may comprise a sequence of SEQ ID NO: 238. In some examples, the mutant soluble ActRIIB-ECD may comprise a sequence of SEQ ID NO: 239. In some examples, the mutant soluble ActRIIB-ECD may comprise a sequence of SEQ ID NO: 240. In some examples, the mutant soluble ActRIIB-ECD may comprise a sequence of SEQ ID NO: 241. In some examples, the mutant soluble ActRIIB-ECD may comprise a sequence of SEQ ID NO: 242. In some examples, the mutant soluble ActRIIB-ECD may comprise a sequence of SEQ ID NO: 243. In some examples, the mutant soluble ActRIIB-ECD may comprise a sequence of SEQ ID NO: 244. In some examples, the mutant soluble ActRIIB-ECD may comprise a sequence of SEQ ID NO: 245. In some examples, the mutant soluble ActRIIB-ECD may comprise a sequence of SEQ ID NO: 246. In some examples, the mutant soluble ActRIIB-ECD may comprise a sequence of SEQ ID NO: 247. In some examples, the mutant soluble ActRIIB-ECD may comprise a sequence of SEQ ID NO: 248. In some examples, the mutant soluble ActRIIB-ECD may comprise a sequence of SEQ ID NO: 249. In some examples, the mutant soluble ActRIIB-ECD may comprise a sequence of SEQ ID NO: 250. In some examples, the mutant soluble ActRIIB-ECD may comprise a sequence of SEQ ID NO: 251. In some examples, the mutant soluble ActRIIB-ECD may comprise a sequence of SEQ ID NO: 252. In some examples, the mutant soluble ActRIIB-ECD may comprise a sequence of SEQ ID NO: 253. In some examples, the mutant soluble ActRIIB-ECD may comprise a sequence of SEQ ID NO: 254. In some examples, the mutant soluble ActRIIB-ECD may comprise a sequence of SEQ ID NO: 255. In some examples, the mutant soluble ActRIIB-ECD may comprise a sequence of SEQ ID NO: 256. In some examples, the mutant soluble ActRIIB-ECD may comprise a sequence of SEQ ID NO: 257. In some examples, the mutant soluble ActRIIB-ECD may comprise a sequence of SEQ ID NO: 258. In some examples, the mutant soluble ActRIIB-ECD may comprise a sequence of SEQ ID NO: 259. In some examples, the mutant soluble ActRIIB-ECD may comprise a sequence of SEQ ID NO: 260. In some examples, the mutant soluble ActRIIB-ECD may comprise a sequence of SEQ ID NO: 261. In some examples, the mutant soluble ActRIIB-ECD may comprise a sequence of SEQ ID NO: 262. In some examples, the mutant soluble ActRIIB-ECD may comprise a sequence of SEQ ID NO: 263. In some examples, the mutant soluble ActRIIB-ECD may comprise a sequence of SEQ ID NO: 264. In some examples, the mutant soluble ActRIIB-ECD may comprise a sequence of SEQ ID NO: 265. In some examples, the mutant soluble ActRIIB-ECD may comprise a sequence of SEQ ID NO: 266. In some examples, the mutant soluble ActRIIB-ECD may comprise a sequence of SEQ ID NO: 267. In some examples, the mutant soluble ActRIIB-ECD may comprise a sequence of SEQ ID NO: 268. In some examples, the mutant soluble ActRIIB-ECD may comprise a sequence of SEQ ID NO: 269. In some examples, the mutant soluble ActRIIB-ECD may comprise a sequence of SEQ ID NO: 270. In some examples, the mutant soluble ActRIIB-ECD may comprise a sequence of SEQ ID NO: 271. In some examples, the mutant soluble ActRIIB-ECD may comprise a sequence of SEQ ID NO: 272. In some examples, the mutant soluble ActRIIB-ECD may comprise a sequence of SEQ ID NO: 273. In some examples, the mutant soluble ActRIIB-ECD may comprise a sequence of SEQ ID NO: 274. In some examples, the mutant soluble ActRIIB-ECD may comprise a sequence of SEQ ID NO: 275. In some examples, the mutant soluble ActRIIB-ECD may comprise a sequence of SEQ ID NO: 276. In some examples, the mutant soluble ActRIIB-ECD may comprise a sequence of SEQ ID NO: 277. In some examples, the mutant soluble ActRIIB-ECD may comprise a sequence of SEQ ID NO: 278. In some examples, the mutant soluble ActRIIB-ECD may comprise a sequence of SEQ ID NO: 279. In some examples, the mutant soluble ActRIIB-ECD may comprise a sequence of SEQ ID NO: 280. In some examples, the mutant soluble ActRIIB-ECD may comprise a sequence of SEQ ID NO: 281. In some examples, the mutant soluble ActRIIB-ECD may comprise a sequence of SEQ ID NO: 282. In some examples, the mutant soluble ActRIIB-ECD may comprise a sequence of SEQ ID NO: 283. In some examples, the mutant soluble ActRIIB-ECD may comprise a sequence of SEQ ID NO: 284. In some examples, the mutant soluble ActRIIB-ECD may comprise a sequence of SEQ ID NO: 285. In some examples, the mutant soluble ActRIIB-ECD may comprise a sequence of SEQ ID NO: 286. In some examples, the mutant soluble ActRIIB-ECD may comprise a sequence of SEQ ID NO: 287. In some examples, the mutant soluble ActRIIB-ECD may comprise a sequence of SEQ ID NO: 288. In some examples, the mutant soluble ActRIIB-ECD may comprise a sequence of SEQ ID NO: 289. In some examples, the mutant soluble ActRIIB-ECD may comprise a sequence of SEQ ID NO: 290. In some examples, the mutant soluble ActRIIB-ECD may comprise a sequence of SEQ ID NO: 291. In some examples, the mutant soluble ActRIIB-ECD may comprise a sequence of SEQ ID NO: 292. In some examples, the mutant soluble ActRIIB-ECD may comprise a sequence of SEQ ID NO: 293. In some examples, the mutant soluble ActRIIB-ECD may comprise a sequence of SEQ ID NO: 294. In some examples, the mutant soluble ActRIIB-ECD may comprise a sequence of SEQ ID NO: 295. In some examples, the mutant soluble ActRIIB-ECD may comprise a sequence of SEQ ID NO: 296. In some examples, the mutant soluble ActRIIB-ECD may comprise a sequence of SEQ ID NO: 297. In some examples, the mutant soluble ActRIIB-ECD may comprise a sequence of SEQ ID NO: 298. In some examples, the mutant soluble ActRIIB-ECD may comprise a sequence of SEQ ID NO: 299. In some examples, the mutant soluble ActRIIB-ECD may comprise a sequence of SEQ ID NO: 300. In some examples, the mutant soluble ActRIIB-ECD may comprise a sequence of SEQ ID NO: 301. In some examples, the mutant soluble ActRIIB-ECD may comprise a sequence of SEQ ID NO: 302. In some examples, the mutant soluble ActRIIB-ECD may comprise a sequence of SEQ ID NO: 303. In some examples, the mutant soluble ActRIIB-ECD may comprise a sequence of SEQ ID NO: 304. In some examples, the mutant soluble ActRIIB-ECD may comprise a sequence of SEQ ID NO: 305. In some examples, the mutant soluble ActRIIB-ECD may comprise a sequence of SEQ ID NO: 306. In some examples, the mutant soluble ActRIIB-ECD may comprise a sequence of SEQ ID NO: 307. In some examples, the mutant soluble ActRIIB-ECD may comprise a sequence of SEQ ID NO: 308. In some examples, the mutant soluble ActRIIB-ECD may comprise a sequence of SEQ ID NO: 309. In some examples, the mutant soluble ActRIIB-ECD may comprise a sequence of SEQ ID NO: 310. In some examples, the mutant soluble ActRIIB-ECD may comprise a sequence of SEQ ID NO: 311. In some examples, the mutant soluble ActRIIB-ECD may comprise a sequence of SEQ ID NO: 312. In some examples, the mutant soluble ActRIIB-ECD may comprise a sequence of SEQ ID NO: 313. In some examples, the mutant soluble ActRIIB-ECD may comprise a sequence of SEQ ID NO: 314. In some examples, the mutant soluble ActRIIB-ECD may comprise a sequence of SEQ ID NO: 315. In some examples, the mutant soluble ActRIIB-ECD may comprise a sequence of SEQ ID NO: 316. In some examples, the mutant soluble ActRIIB-ECD may comprise a sequence of SEQ ID NO: 317. In some examples, the mutant soluble ActRIIB-ECD may comprise a sequence of SEQ ID NO: 318. In some examples, the mutant soluble ActRIIB-ECD may comprise a sequence of SEQ ID NO: 319. In some examples, the mutant soluble ActRIIB-ECD may comprise a sequence of SEQ ID NO: 320. In some examples, the mutant soluble ActRIIB-ECD may comprise a sequence of SEQ ID NO: 321. In some examples, the mutant soluble ActRIIB-ECD may comprise a sequence of SEQ ID NO: 322. In some examples, the mutant soluble ActRIIB-ECD may comprise a sequence of SEQ ID NO: 323. In some examples, the mutant soluble ActRIIB-ECD may comprise a sequence of SEQ ID NO: 324. In some examples, the mutant soluble ActRIIB-ECD may comprise a sequence of SEQ ID NO: 325.

In some embodiments, the isolated protein may comprise a mutant ActRIIB-ECD may comprise a sequence selected from the group consisting of SEQ ID NOs: 224-325, wherein the mutant ActRIIB-ECD may further comprise from 1 to 6 additional amino acids on the N-terminus of the sequence corresponding to amino acids 1-6 of SEQ ID NO: 46.

In some examples, the mutant ActRIIB-ECD may comprise a sequence of SEQ ID NOs: 1666, 393, 399, 405, 411, 417, 423, 429, 435, 441, 447, 453, 459, 465, 471, 477, 483, 489, 495, 501, 507, 513, 519, 525, 531, 537, 543, 549, 555, 561, 567, 573, 579, 585, 591, 597, 603, 609, 615, 621, 627, 633, 639, 645, 651, 657, 663, 669, 675, 681, 687, 693, 699, 705, 711, 717, 723, 729, 735, 741, 747, 753, 759, 765, 771, 777, 783, 789, 795, 801, 807, 813, 819, 825, 831, 837, 843, 849, 855, 861, 867, 873, 879, 885, 891, 897, 903, 909, 915, 921, 927, 933, 939, 945, 951, 957, 963, 969, 975, 981, 987, or 993, in which N19 is substituted with another amino acid. For example, the mutant ActRIIB-ECD may comprise a sequence of SEQ ID NO: 1000, 1006, 1012, 1018, 1024, 1030, 1036, 1042, 1048, 1054, 1060, 1066, 1072, 1078, 1084, 1090, 1096, 1102, 1108, 1114, 1120, 1126, 1132, 1138, 1144, 1150, 1156, 1162, 1168, 1174, 1180, 1186, 1192, 1198, 1204, 1210, 1216, 1222, 1228, 1234, 1240, 1246, 1252, 1258, 1264, 1270, 1276, 1282, 1288, 1294, 1300, 1306, 1312, 1318, 1324, 1330, 1336, 1342, 1348, 1354, 1360, 1366, 1372, 1378, 1384, 1390, 1396, 1402, 1408, 1414, 1420, 1426, 1432, 1438, 1444, 1450, 1456, 1462, 1468, 1474, 1480, 1486, 1492, 1498, 1504, 1510, 1516, 1522, 1528, 1534, 1540, 1546, 1552, 1558, 1564, 1570, 1576, 1582, 1588, 1594, 1600, or 1606.

In some examples, the mutant ActRIIB-ECD may comprise a sequence of SEQ ID NO: 388, 394, 400, 406, 412, 418, 424, 430, 436, 442, 448, 454, 460, 466, 472, 478, 484, 490, 496, 502, 508, 514, 520, 526, 532, 538, 544, 550, 556, 562, 568, 574, 580, 586, 592, 598, 604, 610, 616, 622, 628, 634, 640, 646, 652, 658, 664, 670, 676, 682, 688, 694, 700, 706, 712, 718, 724, 730, 736, 742, 748, 754, 760, 766, 772, 778, 784, 790, 796, 802, 808, 814, 820, 826, 832, 838, 844, 850, 856, 862, 868, 874, 880, 886, 892, 898, 904, 910, 916, 922, 928, 934, 940, 946, 952, 958, 964, 970, 976, 982, 988, or 994, in which N20 is substituted with another amino acid. For example, the mutant ActRIIB-ECD may comprise a sequence of SEQ ID NO: 1001, 1007, 1013, 1019, 1025, 1031, 1037, 1043, 1049, 1055, 1061, 1067, 1073, 1079, 1085, 1091, 1097, 1103, 1109, 1115, 1121, 1127, 1133, 1139, 1145, 1151, 1157, 1163, 1169, 1175, 1181, 1187, 1193, 1199, 1205, 1211, 1217, 1223, 1229, 1235, 1241, 1247, 1253, 1259, 1265, 1271, 1277, 1283, 1289, 1295, 1301, 1307, 1313, 1319, 1325, 1331, 1337, 1343, 1349, 1355, 1361, 1367, 1373, 1379, 1385, 1391, 1397, 1403, 1409, 1415, 1421, 1427, 1433, 1439, 1445, 1451, 1457, 1463, 1469, 1475, 1481, 1487, 1493, 1499, 1505, 1511, 1517, 1523, 1529, 1535, 1541, 1547, 1553, 1559, 1565, 1571, 1577, 1583, 1589, 1595, 1601, or 1607.

In some examples, the mutant ActRIIB-ECD may comprise a sequence of SEQ ID NO: 389, 395, 401, 407, 413, 419, 425, 431, 437, 443, 449, 455, 461, 467, 473, 479, 485, 491, 497, 503, 509, 515, 521, 527, 533, 539, 545, 551, 557, 563, 569, 575, 581, 587, 593, 599, 605, 611, 617, 623, 629, 635, 641, 647, 653, 659, 665, 671, 677, 683, 689, 695, 701, 707, 713, 719, 725, 731, 737, 743, 749, 755, 761, 767, 773, 779, 785, 791, 797, 803, 809, 815, 821, 827, 833, 839, 845, 851, 857, 863, 869, 875, 881, 887, 893, 899, 905, 911, 917, 923, 929, 935, 941, 947, 953, 959, 965, 971, 977, 983, 989, or 995, in which N21 is substituted with another amino acid. For example, the mutant ActRIIB-ECD may comprise a sequence of SEQ ID NO: 1002, 1008, 1014, 1020, 1026, 1032, 1038, 1044, 1050, 1056, 1062, 1068, 1074, 1080, 1086, 1092, 1098, 1104, 1110, 1116, 1122, 1128, 1134, 1140, 1146, 1152, 1158, 1164, 1170, 1176, 1182, 1188, 1194, 1200, 1206, 1212, 1218, 1224, 1230, 1236, 1242, 1248, 1254, 1260, 1266, 1272, 1278, 1284, 1290, 1296, 1302, 1308, 1314, 1320, 1326, 1332, 1338, 1344, 1350, 1356, 1362, 1368, 1374, 1380, 1386, 1392, 1398, 1404, 1410, 1416, 1422, 1428, 1434, 1440, 1446, 1452, 1458, 1464, 1470, 1476, 1482, 1488, 1494, 1500, 1506, 1512, 1518, 1524, 1530, 1536, 1542, 1548, 1554, 1560, 1566, 1572, 1578, 1584, 1590, 1596, 1602, or 1608.

In some examples, the mutant ActRIIB-ECD may comprise a sequence of SEQ ID NO: 390, 396, 402, 408, 414, 420, 426, 432, 438, 444, 450, 456, 462, 468, 474, 480, 486, 492, 498, 504, 510, 516, 522, 528, 534, 540, 546, 552, 558, 564, 570, 576, 582, 588, 594, 600, 606, 612, 618, 624, 630, 636, 642, 648, 654, 660, 666, 672, 678, 684, 690, 696, 702, 708, 714, 720, 726, 732, 738, 744, 750, 756, 762, 768, 774, 780, 786, 792, 798, 804, 810, 816, 822, 828, 834, 840, 846, 852, 858, 864, 870, 876, 882, 888, 894, 900, 906, 912, 918, 924, 930, 936, 942, 948, 954, 960, 966, 972, 978, 984, 990, or 996, in which N22 is substituted with another amino acid. For example, the mutant ActRIIB-ECD may comprise a sequence of SEQ ID NO: 1003, 1009, 1015, 1021, 1027, 1033, 1039, 1045, 1051, 1057, 1063, 1069, 1075, 1081, 1087, 1093, 1099, 1105, 1111, 1117, 1123, 1129, 1135, 1141, 1147, 1153, 1159, 1165, 1171, 1177, 1183, 1189, 1195, 1201, 1207, 1213, 1219, 1225, 1231, 1237, 1243, 1249, 1255, 1261, 1267, 1273, 1279, 1285, 1291, 1297, 1303, 1309, 1315, 1321, 1327, 1333, 1339, 1345, 1351, 1357, 1363, 1369, 1375, 1381, 1387, 1393, 1399, 1405, 1411, 1417, 1423, 1429, 1435, 1441, 1447, 1453, 1459, 1465, 1471, 1477, 1483, 1489, 1495, 1501, 1507, 1513, 1519, 1525, 1531, 1537, 1543, 1549, 1555, 1561, 1567, 1573, 1579, 1585, 1591, 1597, 1603, or 1609.

In some examples, the mutant ActRIIB-ECD may comprise a sequence of SEQ ID NO: 391, 397, 403, 409, 415, 421, 427, 433, 439, 445, 451, 457, 463, 469, 475, 481, 487, 493, 499, 505, 511, 517, 523, 529, 535, 541, 547, 553, 559, 565, 571, 577, 583, 589, 595, 601, 607, 613, 619, 625, 631, 637, 643, 649, 655, 661, 667, 673, 679, 685, 691, 697, 703, 709, 715, 721, 727, 733, 739, 745, 751, 757, 763, 769, 775, 781, 787, 793, 799, 805, 811, 817, 823, 829, 835, 841, 847, 853, 859, 865, 871, 877, 883, 889, 895, 901, 907, 913, 919, 925, 931, 937, 943, 949, 955, 961, 967, 973, 979, 985, 991, or 997, in which N23 is substituted with another amino acid. For example, the mutant ActRIIB-ECD may comprise a sequence of SEQ ID NO: 1004, 1010, 1016, 1022, 1028, 1034, 1040, 1046, 1052, 1058, 1064, 1070, 1076, 1082, 1088, 1094, 1100, 1106, 1112, 1118, 1124, 1130, 1136, 1142, 1148, 1154, 1160, 1166, 1172, 1178, 1184, 1190, 1196, 1202, 1208, 1214, 1220, 1226, 1232, 1238, 1244, 1250, 1256, 1262, 1268, 1274, 1280, 1286, 1292, 1298, 1304, 1310, 1316, 1322, 1328, 1334, 1340, 1346, 1352, 1358, 1364, 1370, 1376, 1382, 1388, 1394, 1400, 1406, 1412, 1418, 1424, 1430, 1436, 1442, 1448, 1454, 1460, 1466, 1472, 1478, 1484, 1490, 1496, 1502, 1508, 1514, 1520, 1526, 1532, 1538, 1544, 1550, 1556, 1562, 1568, 1574, 1580, 1586, 1592, 1598, 1604, or 1610.

In some examples, the mutant ActRIIB-ECD may comprise a sequence of SEQ ID NO: 392, 398, 404, 410, 416, 422, 428, 434, 440, 446, 452, 458, 464, 470, 476, 482, 488, 494, 500, 506, 512, 518, 524, 530, 536, 542, 548, 554, 560, 566, 572, 578, 584, 590, 596, 602, 608, 614, 620, 626, 632, 638, 644, 650, 656, 662, 668, 674, 680, 686, 692, 698, 704, 710, 716, 722, 728, 734, 740, 746, 752, 758, 764, 770, 776, 782, 788, 794, 800, 806, 812, 818, 824, 830, 836, 842, 848, 854, 860, 866, 872, 878, 884, 890, 896, 902, 908, 914, 920, 926, 932, 938, 944, 950, 956, 962, 968, 974, 980, 986, 992, or 998, in which N24 is substituted with another amino acid. For example, the mutant ActRIIB-ECD may comprise a sequence of SEQ ID NO: 1005, 1011, 1017, 1023, 1029, 1035, 1041, 1047, 1053, 1059, 1065, 1071, 1077, 1083, 1089, 1095, 1101, 1107, 1113, 1119, 1125, 1131, 1137, 1143, 1149, 1155, 1161, 1167, 1173, 1179, 1185, 1191, 1197, 1203, 1209, 1215, 1221, 1227, 1233, 1239, 1245, 1251, 1257, 1263, 1269, 1275, 1281, 1287, 1293, 1299, 1305, 1311, 1317, 1323, 1329, 1335, 1341, 1347, 1353, 1359, 1365, 1371, 1377, 1383, 1389, 1395, 1401, 1407, 1413, 1419, 1425, 1431, 1437, 1443, 1449, 1455, 1461, 1467, 1473, 1479, 1485, 1491, 1497, 1503, 1509, 1515, 1521, 1527, 1533, 1539, 1545, 1551, 1557, 1563, 1569, 1575, 1581, 1587, 1593, 1599, 1605, or 1611.

In some examples, the mutant ActRIIB-ECD may comprise a sequence of SEQ ID NO: 1666, 393, 399, 405, 411, 417, 423, 429, 435, 441, 447, 453, 459, 465, 471, 477, 483, 489, 495, 501, 507, 513, 519, 525, 531, 537, 543, 549, 555, 561, 567, 573, 579, 585, 591, 597, 603, 609, 615, 621, 627, 633, 639, 645, 651, 657, 663, 669, 675, 681, 687, 693, 699, 705, 711, 717, 723, 729, 735, 741, 747, 753, 759, 765, 771, 777, 783, 789, 795, 801, 807, 813, 819, 825, 831, 837, 843, 849, 855, 861, 867, 873, 879, 885, 891, 897, 903, 909, 915, 921, 927, 933, 939, 945, 951, 957, 963, 969, 975, 981, 987, or 993, in which S21 is substituted with another amino acid other than serine(S) or threonine (T).

In some examples, the mutant ActRIIB-ECD may comprise a sequence of SEQ ID NO: 388, 394, 400, 406, 412, 418, 424, 430, 436, 442, 448, 454, 460, 466, 472, 478, 484, 490, 496, 502, 508, 514, 520, 526, 532, 538, 544, 550, 556, 562, 568, 574, 580, 586, 592, 598, 604, 610, 616, 622, 628, 634, 640, 646, 652, 658, 664, 670, 676, 682, 688, 694, 700, 706, 712, 718, 724, 730, 736, 742, 748, 754, 760, 766, 772, 778, 784, 790, 796, 802, 808, 814, 820, 826, 832, 838, 844, 850, 856, 862, 868, 874, 880, 886, 892, 898, 904, 910, 916, 922, 928, 934, 940, 946, 952, 958, 964, 970, 976, 982, 988, or 994, in which S22 is substituted with another amino acid other than serine(S) or threonine (T).

In some examples, the mutant ActRIIB-ECD may comprise a sequence of SEQ ID NO: 389, 395, 401, 407, 413, 419, 425, 431, 437, 443, 449, 455, 461, 467, 473, 479, 485, 491, 497, 503, 509, 515, 521, 527, 533, 539, 545, 551, 557, 563, 569, 575, 581, 587, 593, 599, 605, 611, 617, 623, 629, 635, 641, 647, 653, 659, 665, 671, 677, 683, 689, 695, 701, 707, 713, 719, 725, 731, 737, 743, 749, 755, 761, 767, 773, 779, 785, 791, 797, 803, 809, 815, 821, 827, 833, 839, 845, 851, 857, 863, 869, 875, 881, 887, 893, 899, 905, 911, 917, 923, 929, 935, 941, 947, 953, 959, 965, 971, 977, 983, 989, or 995, in which S23 is substituted with another amino acid other than serine(S) or threonine (T).

In some examples, the mutant ActRIIB-ECD may comprise a sequence of SEQ ID NO: 390, 396, 402, 408, 414, 420, 426, 432, 438, 444, 450, 456, 462, 468, 474, 480, 486, 492, 498, 504, 510, 516, 522, 528, 534, 540, 546, 552, 558, 564, 570, 576, 582, 588, 594, 600, 606, 612, 618, 624, 630, 636, 642, 648, 654, 660, 666, 672, 678, 684, 690, 696, 702, 708, 714, 720, 726, 732, 738, 744, 750, 756, 762, 768, 774, 780, 786, 792, 798, 804, 810, 816, 822, 828, 834, 840, 846, 852, 858, 864, 870, 876, 882, 888, 894, 900, 906, 912, 918, 924, 930, 936, 942, 948, 954, 960, 966, 972, 978, 984, 990, or 996, in which S24 is substituted with another amino acid other than serine(S) or threonine (T).

In some examples, the mutant ActRIIB-ECD may comprise a sequence of SEQ ID NO: 391, 397, 403, 409, 415, 421, 427, 433, 439, 445, 451, 457, 463, 469, 475, 481, 487, 493, 499, 505, 511, 517, 523, 529, 535, 541, 547, 553, 559, 565, 571, 577, 583, 589, 595, 601, 607, 613, 619, 625, 631, 637, 643, 649, 655, 661, 667, 673, 679, 685, 691, 697, 703, 709, 715, 721, 727, 733, 739, 745, 751, 757, 763, 769, 775, 781, 787, 793, 799, 805, 811, 817, 823, 829, 835, 841, 847, 853, 859, 865, 871, 877, 883, 889, 895, 901, 907, 913, 919, 925, 931, 937, 943, 949, 955, 961, 967, 973, 979, 985, 991, or 997, in which S25 is substituted with another amino acid other than serine(S) or threonine (T).

In some examples, the mutant ActRIIB-ECD may comprise a sequence of SEQ ID NO: 392, 398, 404, 410, 416, 422, 428, 434, 440, 446, 452, 458, 464, 470, 476, 482, 488, 494, 500, 506, 512, 518, 524, 530, 536, 542, 548, 554, 560, 566, 572, 578, 584, 590, 596, 602, 608, 614, 620, 626, 632, 638, 644, 650, 656, 662, 668, 674, 680, 686, 692, 698, 704, 710, 716, 722, 728, 734, 740, 746, 752, 758, 764, 770, 776, 782, 788, 794, 800, 806, 812, 818, 824, 830, 836, 842, 848, 854, 860, 866, 872, 878, 884, 890, 896, 902, 908, 914, 920, 926, 932, 938, 944, 950, 956, 962, 968, 974, 980, 986, 992, or 998, in which S26 is substituted with another amino acid other than serine(S) or threonine (T).

In some embodiments, the mutant soluble ActRIIB-ECD may consist of a sequence of any one of SEQ ID NOs: 3-37 and 51-117, in which the asparagine at position N18 is substituted with another amino acid. In some examples, the mutant soluble ActRIIB-ECD may consist of a sequence of SEQ ID NO: 224. In some examples, the mutant soluble ActRIIB-ECD may consist of a sequence of SEQ ID NO: 225. In some examples, the mutant soluble ActRIIB-ECD may consist of a sequence of SEQ ID NO: 226. In some examples, the mutant soluble ActRIIB-ECD may consist of a sequence of SEQ ID NO: 227. In some examples, the mutant soluble ActRIIB-ECD may consist of a sequence of SEQ ID NO: 228. In some examples, the mutant soluble ActRIIB-ECD may consist of a sequence of SEQ ID NO: 229. In some examples, the mutant soluble ActRIIB-ECD may consist of a sequence of SEQ ID NO: 230. In some examples, the mutant soluble ActRIIB-ECD may consist of a sequence of SEQ ID NO: 231. In some examples, the mutant soluble ActRIIB-ECD may consist of a sequence of SEQ ID NO: 232. In some examples, the mutant soluble ActRIIB-ECD may consist of a sequence of SEQ ID NO: 233. In some examples, the mutant soluble ActRIIB-ECD may consist of a sequence of SEQ ID NO: 234. In some examples, the mutant soluble ActRIIB-ECD may consist of a sequence of SEQ ID NO: 235. In some examples, the mutant soluble ActRIIB-ECD may consist of a sequence of SEQ ID NO: 236. In some examples, the mutant soluble ActRIIB-ECD may consist of a sequence of SEQ ID NO: 237. In some examples, the mutant soluble ActRIIB-ECD may consist of a sequence of SEQ ID NO: 238. In some examples, the mutant soluble ActRIIB-ECD may consist of a sequence of SEQ ID NO: 239. In some examples, the mutant soluble ActRIIB-ECD may consist of a sequence of SEQ ID NO: 240. In some examples, the mutant soluble ActRIIB-ECD may consist of a sequence of SEQ ID NO: 241. In some examples, the mutant soluble ActRIIB-ECD may consist of a sequence of SEQ ID NO: 242. In some examples, the mutant soluble ActRIIB-ECD may consist of a sequence of SEQ ID NO: 243. In some examples, the mutant soluble ActRIIB-ECD may consist of a sequence of SEQ ID NO: 244. In some examples, the mutant soluble ActRIIB-ECD may consist of a sequence of SEQ ID NO: 245. In some examples, the mutant soluble ActRIIB-ECD may consist of a sequence of SEQ ID NO: 246. In some examples, the mutant soluble ActRIIB-ECD may consist of a sequence of SEQ ID NO: 247. In some examples, the mutant soluble ActRIIB-ECD may consist of a sequence of SEQ ID NO: 248. In some examples, the mutant soluble ActRIIB-ECD may consist of a sequence of SEQ ID NO: 249. In some examples, the mutant soluble ActRIIB-ECD may consist of a sequence of SEQ ID NO: 250. In some examples, the mutant soluble ActRIIB-ECD may consist of a sequence of SEQ ID NO: 251. In some examples, the mutant soluble ActRIIB-ECD may consist of a sequence of SEQ ID NO: 252. In some examples, the mutant soluble ActRIIB-ECD may consist of a sequence of SEQ ID NO: 253. In some examples, the mutant soluble ActRIIB-ECD may consist of a sequence of SEQ ID NO: 254. In some examples, the mutant soluble ActRIIB-ECD may consist of a sequence of SEQ ID NO: 255. In some examples, the mutant soluble ActRIIB-ECD may consist of a sequence of SEQ ID NO: 256. In some examples, the mutant soluble ActRIIB-ECD may consist of a sequence of SEQ ID NO: 257. In some examples, the mutant soluble ActRIIB-ECD may consist of a sequence of SEQ ID NO: 258. In some examples, the mutant soluble ActRIIB-ECD may consist of a sequence of SEQ ID NO: 259. In some examples, the mutant soluble ActRIIB-ECD may consist of a sequence of SEQ ID NO: 260. In some examples, the mutant soluble ActRIIB-ECD may consist of a sequence of SEQ ID NO: 261. In some examples, the mutant soluble ActRIIB-ECD may consist of a sequence of SEQ ID NO: 262. In some examples, the mutant soluble ActRIIB-ECD may consist of a sequence of SEQ ID NO: 263. In some examples, the mutant soluble ActRIIB-ECD may consist of a sequence of SEQ ID NO: 264. In some examples, the mutant soluble ActRIIB-ECD may consist of a sequence of SEQ ID NO: 265. In some examples, the mutant soluble ActRIIB-ECD may consist of a sequence of SEQ ID NO: 266. In some examples, the mutant soluble ActRIIB-ECD may consist of a sequence of SEQ ID NO: 267. In some examples, the mutant soluble ActRIIB-ECD may consist of a sequence of SEQ ID NO: 268. In some examples, the mutant soluble ActRIIB-ECD may consist of a sequence of SEQ ID NO: 269. In some examples, the mutant soluble ActRIIB-ECD may consist of a sequence of SEQ ID NO: 270. In some examples, the mutant soluble ActRIIB-ECD may consist of a sequence of SEQ ID NO: 271. In some examples, the mutant soluble ActRIIB-ECD may consist of a sequence of SEQ ID NO: 272. In some examples, the mutant soluble ActRIIB-ECD may consist of a sequence of SEQ ID NO: 273. In some examples, the mutant soluble ActRIIB-ECD may consist of a sequence of SEQ ID NO: 274. In some examples, the mutant soluble ActRIIB-ECD may consist of a sequence of SEQ ID NO: 275. In some examples, the mutant soluble ActRIIB-ECD may consist of a sequence of SEQ ID NO: 276. In some examples, the mutant soluble ActRIIB-ECD may consist of a sequence of SEQ ID NO: 277. In some examples, the mutant soluble ActRIIB-ECD may consist of a sequence of SEQ ID NO: 278. In some examples, the mutant soluble ActRIIB-ECD may consist of a sequence of SEQ ID NO: 279. In some examples, the mutant soluble ActRIIB-ECD may consist of a sequence of SEQ ID NO: 280. In some examples, the mutant soluble ActRIIB-ECD may consist of a sequence of SEQ ID NO: 281. In some examples, the mutant soluble ActRIIB-ECD may consist of a sequence of SEQ ID NO: 282. In some examples, the mutant soluble ActRIIB-ECD may consist of a sequence of SEQ ID NO: 283. In some examples, the mutant soluble ActRIIB-ECD may consist of a sequence of SEQ ID NO: 284. In some examples, the mutant soluble ActRIIB-ECD may consist of a sequence of SEQ ID NO: 285. In some examples, the mutant soluble ActRIIB-ECD may consist of a sequence of SEQ ID NO: 286. In some examples, the mutant soluble ActRIIB-ECD may consist of a sequence of SEQ ID NO: 287. In some examples, the mutant soluble ActRIIB-ECD may consist of a sequence of SEQ ID NO: 288. In some examples, the mutant soluble ActRIIB-ECD may consist of a sequence of SEQ ID NO: 289. In some examples, the mutant soluble ActRIIB-ECD may consist of a sequence of SEQ ID NO: 290. In some examples, the mutant soluble ActRIIB-ECD may consist of a sequence of SEQ ID NO: 291. In some examples, the mutant soluble ActRIIB-ECD may consist of a sequence of SEQ ID NO: 292. In some examples, the mutant soluble ActRIIB-ECD may consist of a sequence of SEQ ID NO: 293. In some examples, the mutant soluble ActRIIB-ECD may consist of a sequence of SEQ ID NO: 294. In some examples, the mutant soluble ActRIIB-ECD may consist of a sequence of SEQ ID NO: 295. In some examples, the mutant soluble ActRIIB-ECD may consist of a sequence of SEQ ID NO: 296. In some examples, the mutant soluble ActRIIB-ECD may consist of a sequence of SEQ ID NO: 297. In some examples, the mutant soluble ActRIIB-ECD may consist of a sequence of SEQ ID NO: 298. In some examples, the mutant soluble ActRIIB-ECD may consist of a sequence of SEQ ID NO: 299. In some examples, the mutant soluble ActRIIB-ECD may consist of a sequence of SEQ ID NO: 300. In some examples, the mutant soluble ActRIIB-ECD may consist of a sequence of SEQ ID NO: 301. In some examples, the mutant soluble ActRIIB-ECD may consist of a sequence of SEQ ID NO: 302. In some examples, the mutant soluble ActRIIB-ECD may consist of a sequence of SEQ ID NO: 303. In some examples, the mutant soluble ActRIIB-ECD may consist of a sequence of SEQ ID NO: 304. In some examples, the mutant soluble ActRIIB-ECD may consist of a sequence of SEQ ID NO: 305. In some examples, the mutant soluble ActRIIB-ECD may consist of a sequence of SEQ ID NO: 306. In some examples, the mutant soluble ActRIIB-ECD may consist of a sequence of SEQ ID NO: 307. In some examples, the mutant soluble ActRIIB-ECD may consist of a sequence of SEQ ID NO: 308. In some examples, the mutant soluble ActRIIB-ECD may consist of a sequence of SEQ ID NO: 309. In some examples, the mutant soluble ActRIIB-ECD may consist of a sequence of SEQ ID NO: 310. In some examples, the mutant soluble ActRIIB-ECD may consist of a sequence of SEQ ID NO: 311. In some examples, the mutant soluble ActRIIB-ECD may consist of a sequence of SEQ ID NO: 312. In some examples, the mutant soluble ActRIIB-ECD may consist of a sequence of SEQ ID NO: 313. In some examples, the mutant soluble ActRIIB-ECD may consist of a sequence of SEQ ID NO: 314. In some examples, the mutant soluble ActRIIB-ECD may consist of a sequence of SEQ ID NO: 315. In some examples, the mutant soluble ActRIIB-ECD may consist of a sequence of SEQ ID NO: 316. In some examples, the mutant soluble ActRIIB-ECD may consist of a sequence of SEQ ID NO: 317. In some examples, the mutant soluble ActRIIB-ECD may consist of a sequence of SEQ ID NO: 318. In some examples, the mutant soluble ActRIIB-ECD may consist of a sequence of SEQ ID NO: 319. In some examples, the mutant soluble ActRIIB-ECD may consist of a sequence of SEQ ID NO: 320. In some examples, the mutant soluble ActRIIB-ECD may consist of a sequence of SEQ ID NO: 321. In some examples, the mutant soluble ActRIIB-ECD may consist of a sequence of SEQ ID NO: 322. In some examples, the mutant soluble ActRIIB-ECD may consist of a sequence of SEQ ID NO: 323. In some examples, the mutant soluble ActRIIB-ECD may consist of a sequence of SEQ ID NO: 324. In some examples, the mutant soluble ActRIIB-ECD may consist of a sequence of SEQ ID NO: 325.

In some examples, in any of SEQ ID NOs: 224-325, the asparagine at position N18 may be substituted with an alanine. In some examples, in any of SEQ ID NOs: 224-325, the asparagine at position N18 may be substituted with an arginine. In some examples, in any of SEQ ID NOs: 224-325, the asparagine at position N18 may be substituted with an aspartate. In some examples, in any of SEQ ID NOs: 224-325, the asparagine at position N18 may be substituted with a cysteine. In some examples, in any of SEQ ID NOs: 224-325, the asparagine at position N18 may be substituted with a glutamate. In some examples, in any of SEQ ID NOs: 224-325, the asparagine at position N18 may be substituted with a glycine. In some examples, in any of SEQ ID NOs: 224-325, the asparagine at position N18 may be substituted with a histidine. In some examples, in any of SEQ ID NOs: 224-325, the asparagine at position N18 may be substituted with an isoleucine. In some examples, in any of SEQ ID NOs: 224-325, the asparagine at position N18 may be substituted with a leucine. In some examples, in any of SEQ ID NOs: 224-325, the asparagine at position N18 may be substituted with a lysine. In some examples, in any of SEQ ID NOs: 224-325, the asparagine at position N18 may be substituted with a methionine. In some examples, in any of SEQ ID NOs: 224-325, the asparagine at position N18 may be substituted with a phenylalanine. In some examples, in any of SEQ ID NOs: 224-325, the asparagine at position N18 may be substituted with a proline. In some examples, in any of SEQ ID NOs: 224-325, the asparagine at position N18 may be substituted with a serine. In some examples, in any of SEQ ID NOs: 224-325, the asparagine at position N18 may be substituted with a threonine. In some examples, in any of SEQ ID NOs: 224-325, the asparagine at position N18 may be substituted with a tryptophan. In some examples, in any of SEQ ID NOs: 224-325, the asparagine at position N18 may be substituted with a tyrosine. In some examples, in any of SEQ ID NOs: 224-325, the asparagine at position N18 may be substituted with a valine.

In some examples, the mutant soluble ActRIIB-ECD may comprise a sequence at least 80% (e.g., at least 85%, 90%, 95%, 96%, 97%, or 98%) identical to any one of SEQ ID NOs: 3-37 and 51-117, in which the asparagine at position N18 is substituted with another amino acid. For example, the mutant soluble ActRIIB-ECD may comprise a sequence at least 95% identical to any one of SEQ ID NOs: 3-37 and 51-117, in which the asparagine at position N18 is substituted with another amino acid.

In some examples, the mutant soluble ActRIIB-ECD may comprise a sequence at least 95% identical to SEQ ID NO: 3, in which the asparagine at position N18 is substituted with another amino acid. In some examples, the mutant soluble ActRIIB-ECD may comprise a sequence at least 95% identical to SEQ ID NO: 4, in which the asparagine at position N18 is substituted with another amino acid. In some examples, the mutant soluble ActRIIB-ECD may comprise a sequence at least 95% identical to SEQ ID NO: 5, in which the asparagine at position N18 is substituted with another amino acid. In some examples, the mutant soluble ActRIIB-ECD may comprise a sequence at least 95% identical to SEQ ID NO: 6, in which the asparagine at position N18 is substituted with another amino acid. In some examples, the mutant soluble ActRIIB-ECD may comprise a sequence at least 95% identical to SEQ ID NO: 7, in which the asparagine at position N18 is substituted with another amino acid. In some examples, the mutant soluble ActRIIB-ECD may comprise a sequence at least 95% identical to SEQ ID NO: 8, in which the asparagine at position N18 is substituted with another amino acid. In some examples, the mutant soluble ActRIIB-ECD may comprise a sequence at least 95% identical to SEQ ID NO: 9, in which the asparagine at position N18 is substituted with another amino acid. In some examples, the mutant soluble ActRIIB-ECD may comprise a sequence at least 95% identical to SEQ ID NO: 10, in which the asparagine at position N18 is substituted with another amino acid. In some examples, the mutant soluble ActRIIB-ECD polypeptide may comprise a sequence at least 95% identical to SEQ ID NO: 11, in which the asparagine at position N18 is substituted with another amino acid. In some examples, the mutant soluble ActRIIB-ECD polypeptide may comprise a sequence at least 95% identical to SEQ ID NO: 12, in which the asparagine at position N18 is substituted with another amino acid. In some examples, the mutant soluble ActRIIB-ECD polypeptide may comprise a sequence at least 95% identical to SEQ ID NO: 13, in which the asparagine at position N18 is substituted with another amino acid. In some examples, the mutant soluble ActRIIB-ECD polypeptide may comprise a sequence at least 95% identical to SEQ ID NO: 14, in which the asparagine at position N18 is substituted with another amino acid. In some examples, the mutant soluble ActRIIB-ECD polypeptide may comprise a sequence at least 95% identical to SEQ ID NO: 15, in which the asparagine at position N18 is substituted with another amino acid. In some examples, the mutant soluble ActRIIB-ECD polypeptide may comprise a sequence at least 95% identical to SEQ ID NO: 16, in which the asparagine at position N18 is substituted with another amino acid. In some examples, the mutant soluble ActRIIB-ECD polypeptide may comprise a sequence at least 95% identical to SEQ ID NO: 17, in which the asparagine at position N18 is substituted with another amino acid. In some examples, the mutant soluble ActRIIB-ECD polypeptide may comprise a sequence at least 95% identical to SEQ ID NO: 18, in which the asparagine at position N18 is substituted with another amino acid. In some examples, the mutant soluble ActRIIB-ECD polypeptide may comprise a sequence at least 95% identical to SEQ ID NO: 19, in which the asparagine at position N18 is substituted with another amino acid. In some examples, the mutant soluble ActRIIB-ECD polypeptide may comprise a sequence at least 95% identical to SEQ ID NO: 21, in which the soluble ActRIIB-ECD polypeptide may comprise a sequence at least 95% identical to SEQ ID NO: 22, in which the asparagine at position N18 is substituted with another amino acid. In some examples, the mutant soluble ActRIIB-ECD polypeptide may comprise a sequence at least 95% identical to SEQ ID NO: 23, in which the asparagine at position N18 is substituted with another amino acid. In some examples, the mutant soluble ActRIIB-ECD polypeptide may comprise a sequence at least 95% identical to SEQ ID NO: 24, in which the asparagine at position N18 is substituted with another amino acid. In some examples, the mutant soluble ActRIIB-ECD polypeptide may comprise a sequence at least 95% identical to SEQ ID NO: 25, in which the asparagine at position N18 is substituted with another amino acid. In some examples, the mutant soluble ActRIIB-ECD polypeptide may comprise a sequence at least 95% identical to SEQ ID NO: 26, in which the asparagine at position N18 is substituted with another amino acid. In some examples, the mutant soluble ActRIIB-ECD polypeptide may comprise a sequence at least 95% identical to SEQ ID NO: 27, in which the asparagine at position N18 is substituted with another amino acid. In some examples, the mutant soluble ActRIIB-ECD polypeptide may comprise a sequence at least 95% identical to SEQ ID NO: 28, in which the asparagine at position N18 is substituted with another amino acid. In some examples, the mutant soluble ActRIIB-ECD polypeptide may comprise a sequence at least 95% identical to SEQ ID NO: 29, in which the asparagine at position N18 is substituted with another amino acid. In some examples, the mutant soluble ActRIIB-ECD polypeptide may comprise a sequence at least 95% identical to SEQ ID NO: 30, in which the asparagine at position N18 is substituted with another amino acid. In some examples, the mutant soluble ActRIIB-ECD polypeptide may comprise a sequence at least 95% identical to SEQ ID NO: 31, in which the asparagine at position N18 is substituted with another amino acid. In some examples, the mutant soluble ActRIIB-ECD polypeptide may comprise a sequence at least 95% identical to SEQ ID NO: 32, in which the asparagine at position N18 is substituted with another amino acid. In some examples, the mutant soluble ActRIIB-ECD polypeptide may comprise a sequence at least 95% identical to SEQ ID NO: 33, in which the asparagine at position N18 is substituted with another amino acid. In some examples, the mutant soluble ActRIIB-ECD polypeptide may comprise a sequence at least 95% identical to SEQ ID NO: 34, in which the asparagine at position N18 is substituted with another amino acid. In some examples, the mutant soluble ActRIIB-ECD polypeptide may comprise a sequence at least 95% identical to SEQ ID NO: 35, in which the asparagine at position N18 is substituted with another amino acid. In some examples, the mutant soluble ActRIIB-ECD polypeptide may comprise a mutant of a sequence at least 95% identical to SEQ ID NO: 36, in which the asparagine at position N18 is substituted with another amino acid. In some examples, the mutant soluble ActRIIB-ECD polypeptide may comprise a sequence at least 95% identical to SEQ ID NO: 37, in which the asparagine at position N18 is substituted with another amino acid. In some examples, the mutant soluble ActRIIB-ECD polypeptide may comprise a sequence at least 95% identical to SEQ ID NO: 51, in which the asparagine at position N18 is substituted with another amino acid. In some examples, the mutant soluble ActRIIB-ECD polypeptide may comprise a sequence at least 95% identical to SEQ ID NO: 52, in which the asparagine at position N18 is substituted with another amino acid. In some examples, the mutant soluble ActRIIB-ECD polypeptide may comprise a sequence at least 95% identical to SEQ ID NO: 53, in which the asparagine at position N18 is substituted with another amino acid. In some examples, the mutant soluble ActRIIB-ECD polypeptide may comprise a sequence at least 95% identical to SEQ ID NO: 54, in which the asparagine at position N18 is substituted with another amino acid. In some examples, the mutant soluble ActRIIB-ECD polypeptide may comprise a sequence at least 95% identical to SEQ ID NO: 55, in which the asparagine at position N18 is substituted with another amino acid. In some examples, the mutant soluble ActRIIB-ECD polypeptide may comprise a sequence at least 95% identical to SEQ ID NO: 56, in which the asparagine at position N18 is substituted with another amino acid. In some examples, the mutant soluble ActRIIB-ECD polypeptide may comprise a sequence at least 95% identical to SEQ ID NO: 57, in which the asparagine at position N18 is substituted with another amino acid. In some examples, the mutant soluble ActRIIB-ECD polypeptide may comprise a sequence at least 95% identical to SEQ ID NO: 58, in which the asparagine at position N18 is substituted with another amino acid. In some examples, the mutant soluble ActRIIB-ECD polypeptide may comprise a sequence at least 95% identical to SEQ ID NO: 59, in which the asparagine at position N18 is substituted with another amino acid. In some examples, the mutant soluble ActRIIB-ECD polypeptide may comprise a sequence at least 95% identical to SEQ ID NO: 60, in which the asparagine at position N18 is substituted with another amino acid. In some examples, the mutant soluble ActRIIB-ECD polypeptide may comprise a sequence at least 95% identical to SEQ ID NO: 61, in which the asparagine at position N18 is substituted with another amino acid. In some examples, the mutant soluble ActRIIB-ECD polypeptide may comprise a sequence at least 95% identical to SEQ ID NO: 62, in which the asparagine at position N18 is substituted with another amino acid. In some examples, the mutant soluble ActRIIB-ECD polypeptide may comprise a sequence at least 95% identical to SEQ ID NO: 63, in which the asparagine at position N18 is substituted with another amino acid. In some examples, the mutant soluble ActRIIB-ECD polypeptide may comprise a sequence at least 95% identical to SEQ ID NO: 64, in which the asparagine at position N18 is substituted with another amino acid. In some examples, the mutant soluble ActRIIB-ECD polypeptide may comprise a sequence at least 95% identical to SEQ ID NO: 65, in which the asparagine at position N18 is substituted with another amino acid. In some examples, the mutant soluble ActRIIB-ECD polypeptide may comprise a sequence at least 95% identical to SEQ ID NO: 66, in which the asparagine at position N18 is substituted with another amino acid. In some examples, the mutant soluble ActRIIB-ECD polypeptide may comprise a sequence at least 95% identical to SEQ ID NO: 67, in which the asparagine at position N18 is substituted with another amino acid. In some examples, the mutant soluble ActRIIB-ECD polypeptide may comprise a sequence at least 95% identical to SEQ ID NO: 68, in which the asparagine at position N18 is substituted with another amino acid. In some examples, the mutant soluble ActRIIB-ECD polypeptide may comprise a sequence at least 95% identical to SEQ ID NO: 69, in which the asparagine at position N18 is substituted with another amino acid. In some examples, the mutant soluble ActRIIB-ECD polypeptide may comprise a sequence at least 95% identical to SEQ ID NO: 70, in which the asparagine at position N18 is substituted with another amino acid. In some examples, the mutant soluble ActRIIB-ECD polypeptide may comprise a sequence at least 95% identical to SEQ ID NO: 71, in which the asparagine at position N18 is substituted with another amino acid. In some examples, the mutant soluble ActRIIB-ECD polypeptide may comprise a sequence at least 95% identical to SEQ ID NO: 72, in which the asparagine at position N18 is substituted with another amino acid. In some examples, the mutant soluble ActRIIB-ECD polypeptide may comprise a sequence at least 95% identical to SEQ ID NO: 73, in which the asparagine at position N18 is substituted with another amino acid. In some examples, the mutant soluble ActRIIB-ECD polypeptide may comprise a sequence at least 95% identical to SEQ ID NO: 74, in which the soluble ActRIIB-ECD polypeptide may comprise a sequence at least 95% identical to SEQ ID NO: 75, in which the asparagine at position N18 is substituted with another amino acid. In some examples, the mutant soluble ActRIIB-ECD polypeptide may comprise a sequence at least 95% identical to SEQ ID NO: 76, in which the asparagine at position N18 is substituted with another amino acid. In some examples, the mutant soluble ActRIIB-ECD polypeptide may comprise a sequence at least 95% identical to SEQ ID NO: 77, in which the asparagine at position N18 is substituted with another amino acid. In some examples, the mutant soluble ActRIIB-ECD polypeptide may comprise a sequence at least 95% identical to SEQ ID NO: 78, in which the asparagine at position N18 is substituted with another amino acid. In some examples, the mutant soluble ActRIIB-ECD polypeptide may comprise a sequence at least 95% identical to SEQ ID NO: 79, in which the asparagine at position N18 is substituted with another amino acid. In some examples, the mutant soluble ActRIIB-ECD polypeptide may comprise a sequence at least 95% identical to SEQ ID NO: 80, in which the asparagine at position N18 is substituted with another amino acid. In some examples, the mutant soluble ActRIIB-ECD polypeptide may comprise a sequence at least 95% identical to SEQ ID NO: 81, in which the asparagine at position N18 is substituted with another amino acid. In some examples, the mutant soluble ActRIIB-ECD polypeptide may comprise a sequence at least 95% identical to SEQ ID NO: 82, in which the asparagine at position N18 is substituted with another amino acid. In some examples, the mutant soluble ActRIIB-ECD polypeptide may comprise a sequence at least 95% identical to SEQ ID NO: 83, in which the asparagine at position N18 is substituted with another amino acid. In some examples, the mutant soluble ActRIIB-ECD polypeptide may comprise a sequence at least 95% identical to SEQ ID NO: 84, in which the asparagine at position N18 is substituted with another amino acid. In some examples, the mutant soluble ActRIIB-ECD polypeptide may comprise a sequence at least 95% identical to SEQ ID NO: 85, in which the asparagine at position N18 is substituted with another amino acid. In some examples, the mutant soluble ActRIIB-ECD polypeptide may comprise a sequence at least 95% identical to SEQ ID NO: 86, in which the asparagine at position N18 is substituted with another amino acid. In some examples, the mutant soluble ActRIIB-ECD polypeptide may comprise a sequence at least 95% identical to SEQ ID NO: 87, in which the asparagine at position N18 is substituted with another amino acid. In some examples, the mutant soluble ActRIIB-ECD polypeptide may comprise a sequence at least 95% identical to SEQ ID NO: 88, in which the asparagine at position N18 is substituted with another amino acid. In some examples, the mutant soluble ActRIIB-ECD polypeptide may comprise a sequence at least 95% identical to SEQ ID NO: 89, in which the asparagine at position N18 is substituted with another amino acid. In some examples, the mutant soluble ActRIIB-ECD polypeptide may comprise a sequence at least 95% identical to SEQ ID NO: 90, in which the asparagine at position N18 is substituted with another amino acid. In some examples, the mutant soluble ActRIIB-ECD polypeptide may comprise a sequence at least 95% identical to SEQ ID NO: 91, in which the asparagine at position N18 is substituted with another amino acid. In some examples, the mutant soluble ActRIIB-ECD polypeptide may comprise a sequence at least 95% identical to SEQ ID NO: 92, in which the asparagine at position N18 is substituted with another amino acid. In some examples, the mutant soluble ActRIIB-ECD polypeptide may comprise a sequence at least 95% identical to SEQ ID NO: 93, in which the asparagine at position N18 is substituted with another amino acid. In some examples, the mutant soluble ActRIIB-ECD polypeptide may comprise a sequence at least 95% identical to SEQ ID NO: 94, in which the asparagine at position N18 is substituted with another amino acid. In some examples, the mutant soluble ActRIIB-ECD polypeptide may comprise a sequence at least 95% identical to SEQ ID NO: 95, in which the asparagine at position N18 is substituted with another amino acid. In some examples, the mutant soluble ActRIIB-ECD polypeptide may comprise a sequence at least 95% identical to SEQ ID NO: 96, in which the asparagine at position N18 is substituted with another amino acid. In some examples, the mutant soluble ActRIIB-ECD polypeptide may comprise a sequence at least 95% identical to SEQ ID NO: 97, in which the asparagine at position N18 is substituted with another amino acid. In some examples, the mutant soluble ActRIIB-ECD polypeptide may comprise a sequence at least 95% identical to SEQ ID NO: 98, in which the asparagine at position N18 is substituted with another amino acid. In some examples, the mutant soluble ActRIIB-ECD polypeptide may comprise a sequence at least 95% identical to SEQ ID NO: 99, in which the asparagine at position N18 is substituted with another amino acid. In some examples, the mutant soluble ActRIIB-ECD polypeptide may comprise a sequence at least 95% identical to SEQ ID NO: 100, in which the asparagine at position N18 is substituted with another amino acid. In some examples, the mutant soluble ActRIIB-ECD polypeptide may comprise a sequence at least 95% identical to SEQ ID NO: 101, in which the asparagine at position N18 is substituted with another amino acid. In some examples, the mutant soluble ActRIIB-ECD polypeptide may comprise a sequence at least 95% identical to SEQ ID NO: 102, in which the asparagine at position N18 is substituted with another amino acid. In some examples, the mutant soluble ActRIIB-ECD polypeptide may comprise a sequence at least 95% identical to SEQ ID NO: 103, in which the asparagine at position N18 is substituted with another amino acid. In some examples, the mutant soluble ActRIIB-ECD polypeptide may comprise a sequence at least 95% identical to SEQ ID NO: 104, in which the asparagine at position N18 is substituted with another amino acid. In some examples, the mutant soluble ActRIIB-ECD polypeptide may comprise a sequence at least 95% identical to SEQ ID NO: 105, in which the asparagine at position N18 is substituted with another amino acid. In some examples, the mutant soluble ActRIIB-ECD polypeptide may comprise a sequence at least 95% identical to SEQ ID NO: 106, in which the asparagine at position N18 is substituted with another amino acid. In some examples, the mutant soluble ActRIIB-ECD polypeptide may comprise a sequence at least 95% identical to SEQ ID NO: 107, in which the asparagine at position N18 is substituted with another amino acid. In some examples, the mutant soluble ActRIIB-ECD polypeptide may comprise a sequence at least 95% identical to SEQ ID NO: 108, in which the asparagine at position N18 is substituted with another amino acid. In some examples, the mutant soluble ActRIIB-ECD polypeptide may comprise a sequence at least 95% identical to SEQ ID NO: 109, in which the asparagine at position N18 is substituted with another amino acid. In some examples, the mutant soluble ActRIIB-ECD polypeptide may comprise a sequence at least 95% identical to SEQ ID NO: 110, in which the asparagine at position N18 is substituted with another amino acid. In some examples, the mutant soluble ActRIIB-ECD polypeptide may comprise a sequence at least 95% identical to SEQ ID NO: 111, in which the asparagine at position N18 is substituted with another amino acid. In some examples, the mutant soluble ActRIIB-ECD polypeptide may comprise a sequence at least 95% identical to SEQ ID NO: 112, in which the asparagine at position N18 is substituted with another amino acid. In some examples, the mutant soluble ActRIIB-ECD polypeptide may comprise a sequence at least 95% identical to SEQ ID NO: 113, in which the asparagine at position N18 is substituted with another amino acid. In some examples, the mutant soluble ActRIIB-ECD polypeptide may comprise a sequence at least 95% identical to SEQ ID NO: 114, in which the soluble ActRIIB-ECD polypeptide may comprise a sequence at least 95% identical to SEQ ID NO: 115, in which the asparagine at position N18 is substituted with another amino acid. In some examples, the mutant soluble ActRIIB-ECD polypeptide may comprise a sequence at least 95% identical to SEQ ID NO: 116, in which the asparagine at position N18 is substituted with another amino acid. In some examples, the mutant soluble ActRIIB-ECD polypeptide may comprise a sequence at least 95% identical to SEQ ID NO: 117, in which the asparagine at position N18 is substituted with another amino acid.

In some embodiments, in the mutant soluble ActRIIB-ECD that has the additional mutation(s), the asparagine at the position corresponding to N18 of SEQ ID NO: 1 is substituted with a glutamine. In some examples, the mutant soluble ActRIIB-ECD may comprise a sequence at least 80% (e.g., at least 85%, 90%, 95%, 96%, 97%, or 98%) identical to any one of SEQ ID NOs: 3-37 and 51-117, in which the asparagine at position N18 is substituted with glutamine. For example, the mutant soluble ActRIIB-ECD may comprise a sequence at least 95% identical to any one of SEQ ID NOs: 3-37 and 51-117, in which the asparagine at position N18 is substituted with glutamine.

In some examples, the mutant soluble ActRIIB-ECD may comprise a sequence at least 95% identical to SEQ ID NO: 3, in which the asparagine at position N18 is substituted with glutamine. In some examples, the mutant soluble ActRIIB-ECD may comprise a sequence at least 95% identical to SEQ ID NO: 4, in which the asparagine at position N18 is substituted with glutamine. In some examples, the mutant soluble ActRIIB-ECD may comprise a sequence at least 95% identical to SEQ ID NO: 5, in which the asparagine at position N18 is substituted with glutamine. In some examples, the mutant soluble ActRIIB-ECD may comprise a sequence at least 95% identical to SEQ ID NO: 6, in which the asparagine at position N18 is substituted with glutamine. In some examples, the mutant soluble ActRIIB-ECD may comprise a sequence at least 95% identical to SEQ ID NO: 7, in which the asparagine at position N18 is substituted with glutamine. In some examples, the mutant soluble ActRIIB-ECD may comprise a sequence at least 95% identical to SEQ ID NO: 8, in which the asparagine at position N18 is substituted with glutamine. In some examples, the mutant soluble ActRIIB-ECD may comprise a sequence at least 95% identical to SEQ ID NO: 9, in which the asparagine at position N18 is substituted with glutamine. In some examples, the mutant soluble ActRIIB-ECD may comprise a sequence at least 95% identical to SEQ ID NO: 10, in which the asparagine at position N18 is substituted with glutamine. In some examples, the mutant soluble ActRIIB-ECD polypeptide may comprise a sequence at least 95% identical to SEQ ID NO: 11, in which the asparagine at position N18 is substituted with glutamine. In some examples, the mutant soluble ActRIIB-ECD polypeptide may comprise a sequence at least 95% identical to SEQ ID NO: 12, in which the asparagine at position N18 is substituted with glutamine. In some examples, the mutant soluble ActRIIB-ECD polypeptide may comprise a sequence at least 95% identical to SEQ ID NO: 13, in which the asparagine at position N18 is substituted with glutamine. In some examples, the mutant soluble ActRIIB-ECD polypeptide may comprise a sequence at least 95% identical to SEQ ID NO: 14, in which the asparagine at position N18 is substituted with glutamine. In some examples, the mutant soluble ActRIIB-ECD polypeptide may comprise a sequence at least 95% identical to SEQ ID NO: 15, in which the asparagine at position N18 is substituted with glutamine. In some examples, the mutant soluble ActRIIB-ECD polypeptide may comprise a sequence at least 95% identical to SEQ ID NO: 16, in which the asparagine at position N18 is substituted with glutamine. In some examples, the mutant soluble ActRIIB-ECD polypeptide may comprise a sequence at least 95% identical to SEQ ID NO: 17, in which the asparagine at position N18 is substituted with glutamine. In some examples, the mutant soluble ActRIIB-ECD polypeptide may comprise a sequence at least 95% identical to SEQ ID NO: 18, in which the asparagine at position N18 is substituted with glutamine. In some examples, the mutant soluble ActRIIB-ECD polypeptide may comprise a sequence at least 95% identical to SEQ ID NO: 19, in which the asparagine at position N18 is substituted with glutamine. In some examples, the mutant soluble ActRIIB-ECD polypeptide may comprise a sequence at least 95% identical to SEQ ID NO: 21, in which the asparagine at position N18 is substituted with glutamine. In some examples, the mutant soluble ActRIIB-ECD polypeptide may comprise a sequence at least 95% identical to SEQ ID NO: 22, in which the asparagine at position N18 is substituted with glutamine. In some examples, the mutant soluble ActRIIB-ECD polypeptide may comprise a sequence at least 95% identical to SEQ ID NO: 23, in which the asparagine at position N18 is substituted with glutamine. In some examples, the mutant soluble ActRIIB-ECD polypeptide may comprise a sequence at least 95% identical to SEQ ID NO: 24, in which the asparagine at position N18 is substituted with glutamine. In some examples, the mutant soluble ActRIIB-ECD polypeptide may comprise a sequence at least 95% identical to SEQ ID NO: 25, in which the asparagine at position N18 is substituted with glutamine. In some examples, the mutant soluble ActRIIB-ECD polypeptide may comprise a sequence at least 95% identical to SEQ ID NO: 26, in which the asparagine at position N18 is substituted with glutamine. In some examples, the mutant soluble ActRIIB-ECD polypeptide may comprise a sequence at least 95% identical to SEQ ID NO: 27, in which the asparagine at position N18 is substituted with glutamine. In some examples, the mutant soluble ActRIIB-ECD polypeptide may comprise a sequence at least 95% identical to SEQ ID NO: 28, in which the asparagine at position N18 is substituted with glutamine. In some examples, the mutant soluble ActRIIB-ECD polypeptide may comprise a sequence at least 95% identical to SEQ ID NO: 29, in which the asparagine at position N18 is substituted with glutamine. In some examples, the mutant soluble ActRIIB-ECD polypeptide may comprise a sequence at least 95% identical to SEQ ID NO: 30, in which the asparagine at position N18 is substituted with glutamine. In some examples, the mutant soluble ActRIIB-ECD polypeptide may comprise a sequence at least 95% identical to SEQ ID NO: 31, in which the asparagine at position N18 is substituted with glutamine. In some examples, the mutant soluble ActRIIB-ECD polypeptide may comprise a sequence at least 95% identical to SEQ ID NO: 32, in which the asparagine at position N18 is substituted with glutamine. In some examples, the mutant soluble ActRIIB-ECD polypeptide may comprise a sequence at least 95% identical to SEQ ID NO: 33, in which the asparagine at position N18 is substituted with glutamine. In some examples, the mutant soluble ActRIIB-ECD polypeptide may comprise a sequence at least 95% identical to SEQ ID NO: 34, in which the asparagine at position N18 is substituted with another amino acid. In some examples, the mutant soluble ActRIIB-ECD polypeptide may comprise a sequence at least 95% identical to SEQ ID NO: 35, in which the asparagine at position N18 is substituted with glutamine. In some examples, the mutant soluble ActRIIB-ECD polypeptide may comprise a mutant of a sequence at least 95% identical to SEQ ID NO: 36, in which the asparagine at position N18 is substituted with glutamine. In some examples, the mutant soluble ActRIIB-ECD polypeptide may comprise a sequence at least 95% identical to SEQ ID NO: 37, in which the asparagine at position N18 is substituted with glutamine. In some examples, the mutant soluble ActRIIB-ECD polypeptide may comprise a sequence at least 95% identical to SEQ ID NO: 51, in which the asparagine at position N18 is substituted with glutamine. In some examples, the mutant soluble ActRIIB-ECD polypeptide may comprise a sequence at least 95% identical to SEQ ID NO: 52, in which the asparagine at position N18 is substituted with glutamine. In some examples, the mutant soluble ActRIIB-ECD polypeptide may comprise a sequence at least 95% identical to SEQ ID NO: 53, in which the asparagine at position N18 is substituted with glutamine. In some examples, the mutant soluble ActRIIB-ECD polypeptide may comprise a sequence at least 95% identical to SEQ ID NO: 54, in which the asparagine at position N18 is substituted with glutamine. In some examples, the mutant soluble ActRIIB-ECD polypeptide may comprise a sequence at least 95% identical to SEQ ID NO: 55, in which the asparagine at position N18 is substituted with glutamine. In some examples, the mutant soluble ActRIIB-ECD polypeptide may comprise a sequence at least 95% identical to SEQ ID NO: 56, in which the asparagine at position N18 is substituted with glutamine. In some examples, the mutant soluble ActRIIB-ECD polypeptide may comprise a sequence at least 95% identical to SEQ ID NO: 57, in which the asparagine at position N18 is substituted with glutamine. In some examples, the mutant soluble ActRIIB-ECD polypeptide may comprise a sequence at least 95% identical to SEQ ID NO: 58, in which the asparagine at position N18 is substituted with glutamine. In some examples, the mutant soluble ActRIIB-ECD polypeptide may comprise a sequence at least 95% identical to SEQ ID NO: 59, in which the asparagine at position N18 is substituted with glutamine. In some examples, the mutant soluble ActRIIB-ECD polypeptide may comprise a sequence at least 95% identical to SEQ ID NO: 60, in which the asparagine at position N18 is substituted with glutamine. In some examples, the mutant soluble ActRIIB-ECD polypeptide may comprise a sequence at least 95% identical to SEQ ID NO: 61, in which the asparagine at position N18 is substituted with glutamine. In some examples, the mutant soluble ActRIIB-ECD polypeptide may comprise a sequence at least 95% identical to SEQ ID NO: 62, in which the asparagine at position N18 is substituted with glutamine. In some examples, the mutant soluble ActRIIB-ECD polypeptide may comprise a sequence at least 95% identical to SEQ ID NO: 63, in which the asparagine at position N18 is substituted with glutamine. In some examples, the mutant soluble ActRIIB-ECD polypeptide may comprise a sequence at least 95% identical to SEQ ID NO: 64, in which the asparagine at position N18 is substituted with glutamine. In some examples, the mutant soluble ActRIIB-ECD polypeptide may comprise a sequence at least 95% identical to SEQ ID NO: 65, in which the asparagine at position N18 is substituted with glutamine. In some examples, the mutant soluble ActRIIB-ECD polypeptide may comprise a sequence at least 95% identical to SEQ ID NO: 66, in which the asparagine at position N18 is substituted with another amino acid In some examples, the mutant soluble ActRIIB-ECD polypeptide may comprise a sequence at least 95% identical to SEQ ID NO: 67, in which the asparagine at position N18 is substituted with glutamine. In some examples, the mutant soluble ActRIIB-ECD polypeptide may comprise a sequence at least 95% identical to SEQ ID NO: 68, in which the asparagine at position N18 is substituted with glutamine. In some examples, the mutant soluble ActRIIB-ECD polypeptide may comprise a sequence at least 95% identical to SEQ ID NO: 69, in which the asparagine at position N18 is substituted with glutamine. In some examples, the mutant soluble ActRIIB-ECD polypeptide may comprise a sequence at least 95% identical to SEQ ID NO: 70, in which the asparagine at position N18 is substituted with glutamine. In some examples, the mutant soluble ActRIIB-ECD polypeptide may comprise a sequence at least 95% identical to SEQ ID NO: 71, in which the asparagine at position N18 is substituted with glutamine. In some examples, the mutant soluble ActRIIB-ECD polypeptide may comprise a sequence at least 95% identical to SEQ ID NO: 72, in which the asparagine at position N18 is substituted with glutamine. In some examples, the mutant soluble ActRIIB-ECD polypeptide may comprise a sequence at least 95% identical to SEQ ID NO: 73, in which the asparagine at position N18 is substituted with glutamine. In some examples, the mutant soluble ActRIIB-ECD polypeptide may comprise a sequence at least 95% identical to SEQ ID NO: 74, in which the asparagine at position N18 is substituted with glutamine. In some examples, the mutant soluble ActRIIB-ECD polypeptide may comprise a sequence at least 95% identical to SEQ ID NO: 75, in which the asparagine at position N18 is substituted with glutamine. In some examples, the mutant soluble ActRIIB-ECD polypeptide may comprise a sequence at least 95% identical to SEQ ID NO: 76, in which the asparagine at position N18 is substituted with another amino acid. In some examples, the mutant soluble ActRIIB-ECD polypeptide may comprise a sequence at least 95% identical to SEQ ID NO: 77, in which the asparagine at position N18 is substituted with glutamine. In some examples, the mutant soluble ActRIIB-ECD polypeptide may comprise a sequence at least 95% identical to SEQ ID NO: 78, in which the asparagine at position N18 is substituted with glutamine. In some examples, the mutant soluble ActRIIB-ECD polypeptide may comprise a sequence at least 95% identical to SEQ ID NO: 79, in which the asparagine at position N18 is substituted with glutamine. In some examples, the mutant soluble ActRIIB-ECD polypeptide may comprise a sequence at least 95% identical to SEQ ID NO: 80, in which the asparagine at position N18 is substituted with glutamine. In some examples, the mutant soluble ActRIIB-ECD polypeptide may comprise a sequence at least 95% identical to SEQ ID NO: 81, in which the asparagine at position N18 is substituted with glutamine. In some examples, the mutant soluble ActRIIB-ECD polypeptide may comprise a sequence at least 95% identical to SEQ ID NO: 82, in which the asparagine at position N18 is substituted with glutamine. In some examples, the mutant soluble ActRIIB-ECD polypeptide may comprise a sequence at least 95% identical to SEQ ID NO: 83, in which the asparagine at position N18 is substituted with glutamine. In some examples, the mutant soluble ActRIIB-ECD polypeptide may comprise a sequence at least 95% identical to SEQ ID NO: 84, in which the asparagine at position N18 is substituted with glutamine. In some examples, the mutant soluble ActRIIB-ECD polypeptide may comprise a sequence at least 95% identical to SEQ ID NO: 85, in which the asparagine at position N18 is substituted with another amino acid. In some examples, the mutant soluble ActRIIB-ECD polypeptide may comprise a sequence at least 95% identical to SEQ ID NO: 86, in which the asparagine at position N18 is substituted with glutamine. In some examples, the mutant soluble ActRIIB-ECD polypeptide may comprise a sequence at least 95% identical to SEQ ID NO: 87, in which the asparagine at position N18 is substituted with another amino acid. In some examples, the mutant soluble ActRIIB-ECD polypeptide may comprise a sequence at least 95% identical to SEQ ID NO: 88, in which the asparagine at position N18 is substituted with glutamine. In some examples, the mutant soluble ActRIIB-ECD polypeptide may comprise a sequence at least 95% identical to SEQ ID NO: 89, in which the asparagine at position N18 is substituted with glutamine. In some examples, the mutant soluble ActRIIB-ECD polypeptide may comprise a sequence at least 95% identical to SEQ ID NO: 90, in which the asparagine at position N18 is substituted with glutamine. In some examples, the mutant soluble ActRIIB-ECD polypeptide may comprise a sequence at least 95% identical to SEQ ID NO: 91, in which the asparagine at position N18 is substituted with glutamine. In some examples, the mutant soluble ActRIIB-ECD polypeptide may comprise a sequence at least 95% identical to SEQ ID NO: 92, in which the asparagine at position N18 is substituted with glutamine. In some examples, the mutant soluble ActRIIB-ECD polypeptide may comprise a sequence at least 95% identical to SEQ ID NO: 93, in which the asparagine at position N18 is substituted with glutamine. In some examples, the mutant soluble ActRIIB-ECD polypeptide may comprise a sequence at least 95% identical to SEQ ID NO: 94, in which the asparagine at position N18 is substituted with glutamine. In some examples, the mutant soluble ActRIIB-ECD polypeptide may comprise a sequence at least 95% identical to SEQ ID NO: 95, in which the asparagine at position N18 is substituted with glutamine. In some examples, the mutant soluble ActRIIB-ECD polypeptide may comprise a sequence at least 95% identical to SEQ ID NO: 96, in which the asparagine at position N18 is substituted with glutamine. In some examples, the mutant soluble ActRIIB-ECD polypeptide may comprise a sequence at least 95% identical to SEQ ID NO: 97, in which the asparagine at position N18 is substituted with glutamine. In some examples, the mutant soluble ActRIIB-ECD polypeptide may comprise a sequence at least 95% identical to SEQ ID NO: 98, in which the asparagine at position N18 is substituted with glutamine. In some examples, the mutant soluble ActRIIB-ECD polypeptide may comprise a sequence at least 95% identical to SEQ ID NO: 99, in which the asparagine at position N18 is substituted with glutamine. In some examples, the mutant soluble ActRIIB-ECD polypeptide may comprise a sequence at least 95% identical to SEQ ID NO: 100, in which the asparagine at position N18 is substituted with glutamine. In some examples, the mutant soluble ActRIIB-ECD polypeptide may comprise a sequence at least 95% identical to SEQ ID NO: 101, in which the asparagine at position N18 is substituted with glutamine. In some examples, the mutant soluble ActRIIB-ECD polypeptide may comprise a sequence at least 95% identical to SEQ ID NO: 102, in which the asparagine at position N18 is substituted with glutamine. In some examples, the mutant soluble ActRIIB-ECD polypeptide may comprise a sequence at least 95% identical to SEQ ID NO: 103, in which the asparagine at position N18 is substituted with glutamine. In some examples, the mutant soluble ActRIIB-ECD polypeptide may comprise a sequence at least 95% identical to SEQ ID NO: 104, in which the asparagine at position N18 is substituted with glutamine. In some examples, the mutant soluble ActRIIB-ECD polypeptide may comprise a sequence at least 95% identical to SEQ ID NO: 105, in which the asparagine at position N18 is substituted with glutamine. In some examples, the mutant soluble ActRIIB-ECD polypeptide may comprise a sequence at least 95% identical to SEQ ID NO: 106, in which the asparagine at position N18 is substituted with glutamine. In some examples, the mutant soluble ActRIIB-ECD polypeptide may comprise a sequence at least 95% identical to SEQ ID NO: 107, in which the asparagine at position N18 is substituted with glutamine. In some examples, the mutant soluble ActRIIB-ECD polypeptide may comprise a sequence at least 95% identical to SEQ ID NO: 108, in which the asparagine at position N18 is substituted with another amino acid. In some examples, the mutant soluble ActRIIB-ECD polypeptide may comprise a sequence at least 95% identical to SEQ ID NO: 109, in which the asparagine at position N18 is substituted with glutamine. In some examples, the mutant soluble ActRIIB-ECD polypeptide may comprise a sequence at least 95% identical to SEQ ID NO: 110, in which the asparagine at position N18 is substituted with glutamine. In some examples, the mutant soluble ActRIIB-ECD polypeptide may comprise a sequence at least 95% identical to SEQ ID NO: 111, in which the asparagine at position N18 is substituted with glutamine. In some examples, the mutant soluble ActRIIB-ECD polypeptide may comprise a sequence at least 95% identical to SEQ ID NO: 112, in which the asparagine at position N18 is substituted with glutamine. In some examples, the mutant soluble ActRIIB-ECD polypeptide may comprise a sequence at least 95% identical to SEQ ID NO: 113, in which the asparagine at position N18 is substituted with glutamine. In some examples, the mutant soluble ActRIIB-ECD polypeptide may comprise a sequence at least 95% identical to SEQ ID NO: 114, in which the asparagine at position N18 is substituted with glutamine. In some examples, the mutant soluble ActRIIB-ECD polypeptide may comprise a sequence at least 95% identical to SEQ ID NO: 115, in which the asparagine at position N18 is substituted with glutamine. In some examples, the mutant soluble ActRIIB-ECD polypeptide may comprise a sequence at least 95% identical to SEQ ID NO: 116, in which the asparagine at position N18 is substituted with glutamine. In some examples, the mutant soluble ActRIIB-ECD polypeptide may comprise a sequence at least 95% identical to SEQ ID NO: 117, in which the asparagine at position N18 is substituted with glutamine.

In some examples, the mutant soluble ActRIIB-ECD may comprise a sequence of any one of SEQ ID NOs: 3-37 and 51-117, in which the asparagine at position N18 is substituted with glutamine. For example, the mutant soluble ActRIIB-ECD may comprise a sequence of any one of SEQ ID NOs: 120-221. In one example, the mutant soluble ActRIIB-ECD may comprise a sequence of SEQ ID NO: 120. In another example, the mutant soluble ActRIIB-ECD may comprise a sequence of SEQ ID NO: 121. In another example, the mutant soluble ActRIIB-ECD may comprise a sequence of SEQ ID NO: 122. In another example, the mutant soluble ActRIIB-ECD may comprise a sequence of SEQ ID NO: 123. In another example, the mutant soluble ActRIIB-ECD may comprise a sequence of SEQ ID NO: 124. In another example, the mutant soluble ActRIIB-ECD may comprise a sequence of SEQ ID NO: 125. In another example, the mutant soluble ActRIIB-ECD may comprise a sequence of SEQ ID NO: 126. In another example, the mutant soluble ActRIIB-ECD may comprise a sequence of SEQ ID NO: 127. In another example, the mutant soluble ActRIIB-ECD may comprise a sequence of SEQ ID NO: 128. In another example, the mutant soluble ActRIIB-ECD may comprise a sequence of SEQ ID NO: 129. In another example, the mutant soluble ActRIIB-ECD may comprise a sequence of SEQ ID NO: 130. In another example, the mutant soluble ActRIIB-ECD may comprise a sequence of SEQ ID NO: 131. In another example, the mutant soluble ActRIIB-ECD may comprise a sequence of SEQ ID NO: 132. In another example, the mutant soluble ActRIIB-ECD may comprise a sequence of SEQ ID NO: 133. In another example, the mutant soluble ActRIIB-ECD may comprise a sequence of SEQ ID NO: 134. In another example, the mutant soluble ActRIIB-ECD may comprise a sequence of SEQ ID NO: 135. In another example, the mutant soluble ActRIIB-ECD may comprise a sequence of SEQ ID NO: 136. In another example, the mutant soluble ActRIIB-ECD may comprise a sequence of SEQ ID NO: 137. In another example, the mutant soluble ActRIIB-ECD may comprise a sequence of SEQ ID NO: 138. In another example, the mutant soluble ActRIIB-ECD may comprise a sequence of SEQ ID NO: 139. In another example, the mutant soluble ActRIIB-ECD may comprise a sequence of SEQ ID NO: 140. In another example, the mutant soluble ActRIIB-ECD may comprise a sequence of SEQ ID NO: 141. In another example, the mutant soluble ActRIIB-ECD may comprise a sequence of SEQ ID NO: 142. In another example, the mutant soluble ActRIIB-ECD may comprise a sequence of SEQ ID NO: 143. In another example, the mutant soluble ActRIIB-ECD may comprise a sequence of SEQ ID NO: 144. In another example, the mutant soluble ActRIIB-ECD may comprise a sequence of SEQ ID NO: 145. In another example, the mutant soluble ActRIIB-ECD may comprise a sequence of SEQ ID NO: 146. In another example, the mutant soluble ActRIIB-ECD may comprise a sequence of SEQ ID NO: 147. In another example, the mutant soluble ActRIIB-ECD may comprise a sequence of SEQ ID NO: 148. In another example, the mutant soluble ActRIIB-ECD may comprise a sequence of SEQ ID NO: 149. In another example, the mutant soluble ActRIIB-ECD may comprise a sequence of SEQ ID NO: 150. In another example, the mutant soluble ActRIIB-ECD may comprise a sequence of SEQ ID NO: 151. In another example, the mutant soluble ActRIIB-ECD may comprise a sequence of SEQ ID NO: 152. In another example, the mutant soluble ActRIIB-ECD may comprise a sequence of SEQ ID NO: 153. In another example, the mutant soluble ActRIIB-ECD may comprise a sequence of SEQ ID NO: 154. In another example, the mutant soluble ActRIIB-ECD may comprise a sequence of SEQ ID NO: 155. In another example, the mutant soluble ActRIIB-ECD may comprise a sequence of SEQ ID NO: 156. In another example, the mutant soluble ActRIIB-ECD may comprise a sequence of SEQ ID NO: 157. In another example, the mutant soluble ActRIIB-ECD may comprise a sequence of SEQ ID NO: 158. In another example, the mutant soluble ActRIIB-ECD may comprise a sequence of SEQ ID NO: 159. In another example, the mutant soluble ActRIIB-ECD may comprise a sequence of SEQ ID NO: 160. In another example, the mutant soluble ActRIIB-ECD may comprise a sequence of SEQ ID NO: 161. In another example, the mutant soluble ActRIIB-ECD may comprise a sequence of SEQ ID NO: 162. In another example, the mutant soluble ActRIIB-ECD may comprise a sequence of SEQ ID NO: 163. In another example, the mutant soluble ActRIIB-ECD may comprise a sequence of SEQ ID NO: 164. In another example, the mutant soluble ActRIIB-ECD may comprise a sequence of SEQ ID NO: 165. In another example, the mutant soluble ActRIIB-ECD may comprise a sequence of SEQ ID NO: 166. In another example, the mutant soluble ActRIIB-ECD may comprise a sequence of SEQ ID NO: 167. In another example, the mutant soluble ActRIIB-ECD may comprise a sequence of SEQ ID NO: 168. In another example, the mutant soluble ActRIIB-ECD may comprise a sequence of SEQ ID NO: 169. In another example, the mutant soluble ActRIIB-ECD may comprise a sequence of SEQ ID NO: 170. In another example, the mutant soluble ActRIIB-ECD may comprise a sequence of SEQ ID NO: 171. In another example, the mutant soluble ActRIIB-ECD may comprise a sequence of SEQ ID NO: 172. In another example, the mutant soluble ActRIIB-ECD may comprise a sequence of SEQ ID NO: 173. In another example, the mutant soluble ActRIIB-ECD may comprise a sequence of SEQ ID NO: 174. In another example, the mutant soluble ActRIIB-ECD may comprise a sequence of SEQ ID NO: 175. In another example, the mutant soluble ActRIIB-ECD may comprise a sequence of SEQ ID NO: 176. In another example, the mutant soluble ActRIIB-ECD may comprise a sequence of SEQ ID NO: 177. In another example, the mutant soluble ActRIIB-ECD may comprise a sequence of SEQ ID NO: 178. In another example, the mutant soluble ActRIIB-ECD may comprise a sequence of SEQ ID NO: 179. In another example, the mutant soluble ActRIIB-ECD may comprise a sequence of SEQ ID NO: 180. In another example, the mutant soluble ActRIIB-ECD may comprise a sequence of SEQ ID NO: 181. In another example, the mutant soluble ActRIIB-ECD may comprise a sequence of SEQ ID NO: 182. In another example, the mutant soluble ActRIIB-ECD may comprise a sequence of SEQ ID NO: 183. In another example, the mutant soluble ActRIIB-ECD may comprise a sequence of SEQ ID NO: 184. In another example, the mutant soluble ActRIIB-ECD may comprise a sequence of SEQ ID NO: 185. In another example, the mutant soluble ActRIIB-ECD may comprise a sequence of SEQ ID NO: 186. In another example, the mutant soluble ActRIIB-ECD may comprise a sequence of SEQ ID NO: 187. In another example, the mutant soluble ActRIIB-ECD may comprise a sequence of SEQ ID NO: 188. In another example, the mutant soluble ActRIIB-ECD may comprise a sequence of SEQ ID NO: 189. In another example, the mutant soluble ActRIIB-ECD may comprise a sequence of SEQ ID NO: 190. In another example, the mutant soluble ActRIIB-ECD may comprise a sequence of SEQ ID NO: 191. In another example, the mutant soluble ActRIIB-ECD may comprise a sequence of SEQ ID NO: 192. In another example, the mutant soluble ActRIIB-ECD may comprise a sequence of SEQ ID NO: 193. In another example, the mutant soluble ActRIIB-ECD may comprise a sequence of SEQ ID NO: 194. In another example, the mutant soluble ActRIIB-ECD may comprise a sequence of SEQ ID NO: 195. In another example, the mutant soluble ActRIIB-ECD may comprise a sequence of SEQ ID NO: 196. In another example, the mutant soluble ActRIIB-ECD may comprise a sequence of SEQ ID NO: 197. In another example, the mutant soluble ActRIIB-ECD may comprise a sequence of SEQ ID NO: 198. In another example, the mutant soluble ActRIIB-ECD may comprise a sequence of SEQ ID NO: 199. In another example, the mutant soluble ActRIIB-ECD may comprise a sequence of SEQ ID NO: 200. In another example, the mutant soluble ActRIIB-ECD may comprise a sequence of SEQ ID NO: 201. In another example, the mutant soluble ActRIIB-ECD may comprise a sequence of SEQ ID NO: 202. In another example, the mutant soluble ActRIIB-ECD may comprise a sequence of SEQ ID NO: 203. In another example, the mutant soluble ActRIIB-ECD may comprise a sequence of SEQ ID NO: 204. In another example, the mutant soluble ActRIIB-ECD may comprise a sequence of SEQ ID NO: 205. In another example, the mutant soluble ActRIIB-ECD may comprise a sequence of SEQ ID NO: 206. In another example, the mutant soluble ActRIIB-ECD may comprise a sequence of SEQ ID NO: 207. In another example, the mutant soluble ActRIIB-ECD may comprise a sequence of SEQ ID NO: 208. In another example, the mutant soluble ActRIIB-ECD may comprise a sequence of SEQ ID NO: 209. In another example, the mutant soluble ActRIIB-ECD may comprise a sequence of SEQ ID NO: 210. In another example, the mutant soluble ActRIIB-ECD may comprise a sequence of SEQ ID NO: 211. In another example, the mutant soluble ActRIIB-ECD may comprise a sequence of SEQ ID NO: 212. In another example, the mutant soluble ActRIIB-ECD may comprise a sequence of SEQ ID NO: 213. In another example, the mutant soluble ActRIIB-ECD may comprise a sequence of SEQ ID NO: 214. In another example, the mutant soluble ActRIIB-ECD may comprise a sequence of SEQ ID NO: 215. In another example, the mutant soluble ActRIIB-ECD may comprise a sequence of SEQ ID NO: 216. In another example, the mutant soluble ActRIIB-ECD may comprise a sequence of SEQ ID NO: 217. In another example, the mutant soluble ActRIIB-ECD may comprise a sequence of SEQ ID NO: 218. In another example, the mutant soluble ActRIIB-ECD may comprise a sequence of SEQ ID NO: 219. In another example, the mutant soluble ActRIIB-ECD may comprise a sequence of SEQ ID NO: 220. In another example, the mutant soluble ActRIIB-ECD may comprise a sequence of SEQ ID NO: 221. In some embodiments, the isolated protein may comprise a mutant ActRIIB-ECD, which may comprise a sequence selected from the group consisting of SEQ ID NOs: 120-221, and the ActRIIB-ECD may further comprise from 1 to 6 additional amino acids on the N-terminus of the sequence corresponding to amino acids 1-6 of SEQ ID NO: 46.

In some examples, the mutant soluble ActRIIB-ECD may consist of a sequence of any one of SEQ ID NOs: 3-37 and 51-117, in which the asparagine at position N18 is substituted with glutamine. For example, the mutant soluble ActRIIB-ECD may consist of a sequence of any one of SEQ ID NOs: 120-221. In one example, the mutant soluble ActRIIB-ECD may consist of a sequence of SEQ ID NO: 120. In another example, the mutant soluble ActRIIB-ECD may consist of a sequence of SEQ ID NO: 121. In another example, the mutant soluble ActRIIB-ECD may consist of a sequence of SEQ ID NO: 122. In another example, the mutant soluble ActRIIB-ECD may consist of a sequence of SEQ ID NO: 123. In another example, the mutant soluble ActRIIB-ECD may consist of a sequence of SEQ ID NO: 124. In another example, the mutant soluble ActRIIB-ECD may consist of a sequence of SEQ ID NO: 125. In another example, the mutant soluble ActRIIB-ECD may consist of a sequence of SEQ ID NO: 126. In another example, the mutant soluble ActRIIB-ECD may consist of a sequence of SEQ ID NO: 127. In another example, the mutant soluble ActRIIB-ECD may consist of a sequence of SEQ ID NO: 128. In another example, the mutant soluble ActRIIB-ECD may consist of a sequence of SEQ ID NO: 129. In another example, the mutant soluble ActRIIB-ECD may consist of a sequence of SEQ ID NO: 130. In another example, the mutant soluble ActRIIB-ECD may consist of a sequence of SEQ ID NO: 131. In another example, the mutant soluble ActRIIB-ECD may consist of a sequence of SEQ ID NO: 132. In another example, the mutant soluble ActRIIB-ECD may consist of a sequence of SEQ ID NO: 133. In another example, the mutant soluble ActRIIB-ECD may consist of a sequence of SEQ ID NO: 134. In another example, the mutant soluble ActRIIB-ECD may consist of a sequence of SEQ ID NO: 135. In another example, the mutant soluble ActRIIB-ECD may consist of a sequence of SEQ ID NO: 136. In another example, the mutant soluble ActRIIB-ECD may consist of a sequence of SEQ ID NO: 137. In another example, the mutant soluble ActRIIB-ECD may consist of a sequence of SEQ ID NO: 138. In another example, the mutant soluble ActRIIB-ECD may consist of a sequence of SEQ ID NO: 139. In another example, the mutant soluble ActRIIB-ECD may consist of a sequence of SEQ ID NO: 140. In another example, the mutant soluble ActRIIB-ECD may consist of a sequence of SEQ ID NO: 141. In another example, the mutant soluble ActRIIB-ECD may consist of a sequence of SEQ ID NO: 142. In another example, the mutant soluble ActRIIB-ECD may consist of a sequence of SEQ ID NO: 143. In another example, the mutant soluble ActRIIB-ECD may consist of a sequence of SEQ ID NO: 144. In another example, the mutant soluble ActRIIB-ECD may consist of a sequence of SEQ ID NO: 145. In another example, the mutant soluble ActRIIB-ECD may consist of a sequence of SEQ ID NO: 146. In another example, the mutant soluble ActRIIB-ECD may consist of a sequence of SEQ ID NO: 147. In another example, the mutant soluble ActRIIB-ECD may consist of a sequence of SEQ ID NO: 148. In another example, the mutant soluble ActRIIB-ECD may consist of a sequence of SEQ ID NO: 149. In another example, the mutant soluble ActRIIB-ECD may consist of a sequence of SEQ ID NO: 150. In another example, the mutant soluble ActRIIB-ECD may consist of a sequence of SEQ ID NO: 151. In another example, the mutant soluble ActRIIB-ECD may consist of a sequence of SEQ ID NO: 152. In another example, the mutant soluble ActRIIB-ECD may consist of a sequence of SEQ ID NO: 153. In another example, the mutant soluble ActRIIB-ECD may consist of a sequence of SEQ ID NO: 154. In another example, the mutant soluble ActRIIB-ECD may consist of a sequence of SEQ ID NO: 155. In another example, the mutant soluble ActRIIB-ECD may consist of a sequence of SEQ ID NO: 156. In another example, the mutant soluble ActRIIB-ECD may consist of a sequence of SEQ ID NO: 157. In another example, the mutant soluble ActRIIB-ECD may consist of a sequence of SEQ ID NO: 158. In another example, the mutant soluble ActRIIB-ECD may consist of a sequence of SEQ ID NO: 159. In another example, the mutant soluble ActRIIB-ECD may consist of a sequence of SEQ ID NO: 160. In another example, the mutant soluble ActRIIB-ECD may consist of a sequence of SEQ ID NO: 161. In another example, the mutant soluble ActRIIB-ECD may consist of a sequence of SEQ ID NO: 162. In another example, the mutant soluble ActRIIB-ECD may consist of a sequence of SEQ ID NO: 163. In another example, the mutant soluble ActRIIB-ECD may consist of a sequence of SEQ ID NO: 164. In another example, the mutant soluble ActRIIB-ECD may consist of a sequence of SEQ ID NO: 165. In another example, the mutant soluble ActRIIB-ECD may consist of a sequence of SEQ ID NO: 166. In another example, the mutant soluble ActRIIB-ECD may consist of a sequence of SEQ ID NO: 167. In another example, the mutant soluble ActRIIB-ECD may consist of a sequence of SEQ ID NO: 168. In another example, the mutant soluble ActRIIB-ECD may consist of a sequence of SEQ ID NO: 169. In another example, the mutant soluble ActRIIB-ECD may consist of a sequence of SEQ ID NO: 170. In another example, the mutant soluble ActRIIB-ECD may consist of a sequence of SEQ ID NO: 171. In another example, the mutant soluble ActRIIB-ECD may consist of a sequence of SEQ ID NO: 172. In another example, the mutant soluble ActRIIB-ECD may consist of a sequence of SEQ ID NO: 173. In another example, the mutant soluble ActRIIB-ECD may consist of a sequence of SEQ ID NO: 174. In another example, the mutant soluble ActRIIB-ECD may consist of a sequence of SEQ ID NO: 175. In another example, the mutant soluble ActRIIB-ECD may consist of a sequence of SEQ ID NO: 176. In another example, the mutant soluble ActRIIB-ECD may consist of a sequence of SEQ ID NO: 177. In another example, the mutant soluble ActRIIB-ECD may consist of a sequence of SEQ ID NO: 178. In another example, the mutant soluble ActRIIB-ECD may consist of a sequence of SEQ ID NO: 179. In another example, the mutant soluble ActRIIB-ECD may consist of a sequence of SEQ ID NO: 180. In another example, the mutant soluble ActRIIB-ECD may consist of a sequence of SEQ ID NO: 181. In another example, the mutant soluble ActRIIB-ECD may consist of a sequence of SEQ ID NO: 182. In another example, the mutant soluble ActRIIB-ECD may consist of a sequence of SEQ ID NO: 183. In another example, the mutant soluble ActRIIB-ECD may consist of a sequence of SEQ ID NO: 184. In another example, the mutant soluble ActRIIB-ECD may consist of a sequence of SEQ ID NO: 185. In another example, the mutant soluble ActRIIB-ECD may consist of a sequence of SEQ ID NO: 186. In another example, the mutant soluble ActRIIB-ECD may consist of a sequence of SEQ ID NO: 187. In another example, the mutant soluble ActRIIB-ECD may consist of a sequence of SEQ ID NO: 188. In another example, the mutant soluble ActRIIB-ECD may consist of a sequence of SEQ ID NO: 189. In another example, the mutant soluble ActRIIB-ECD may consist of a sequence of SEQ ID NO: 190. In another example, the mutant soluble ActRIIB-ECD may consist of a sequence of SEQ ID NO: 191. In another example, the mutant soluble ActRIIB-ECD may consist of a sequence of SEQ ID NO: 192. In another example, the mutant soluble ActRIIB-ECD may consist of a sequence of SEQ ID NO: 193. In another example, the mutant soluble ActRIIB-ECD may consist of a sequence of SEQ ID NO: 194. In another example, the mutant soluble ActRIIB-ECD may consist of a sequence of SEQ ID NO: 195. In another example, the mutant soluble ActRIIB-ECD may consist of a sequence of SEQ ID NO: 196. In another example, the mutant soluble ActRIIB-ECD may consist of a sequence of SEQ ID NO: 197. In another example, the mutant soluble ActRIIB-ECD may consist of a sequence of SEQ ID NO: 198. In another example, the mutant soluble ActRIIB-ECD may consist of a sequence of SEQ ID NO: 199. In another example, the mutant soluble ActRIIB-ECD may consist of a sequence of SEQ ID NO: 200. In another example, the mutant soluble ActRIIB-ECD may consist of a sequence of SEQ ID NO: 201. In another example, the mutant soluble ActRIIB-ECD may consist of a sequence of SEQ ID NO: 202. In another example, the mutant soluble ActRIIB-ECD may consist of a sequence of SEQ ID NO: 203. In another example, the mutant soluble ActRIIB-ECD may consist of a sequence of SEQ ID NO: 204. In another example, the mutant soluble ActRIIB-ECD may consist of a sequence of SEQ ID NO: 205. In another example, the mutant soluble ActRIIB-ECD may consist of a sequence of SEQ ID NO: 206. In another example, the mutant soluble ActRIIB-ECD may consist of a sequence of SEQ ID NO: 207. In another example, the mutant soluble ActRIIB-ECD may consist of a sequence of SEQ ID NO: 208. In another example, the mutant soluble ActRIIB-ECD may consist of a sequence of SEQ ID NO: 209. In another example, the mutant soluble ActRIIB-ECD may consist of a sequence of SEQ ID NO: 210. In another example, the mutant soluble ActRIIB-ECD may consist of a sequence of SEQ ID NO: 211. In another example, the mutant soluble ActRIIB-ECD may consist of a sequence of SEQ ID NO: 212. In another example, the mutant soluble ActRIIB-ECD may consist of a sequence of SEQ ID NO: 213. In another example, the mutant soluble ActRIIB-ECD may consist of a sequence of SEQ ID NO: 214. In another example, the mutant soluble ActRIIB-ECD may consist of a sequence of SEQ ID NO: 215. In another example, the mutant soluble ActRIIB-ECD may consist of a sequence of SEQ ID NO: 216. In another example, the mutant soluble ActRIIB-ECD may consist of a sequence of SEQ ID NO: 217. In another example, the mutant soluble ActRIIB-ECD may consist of a sequence of SEQ ID NO: 218. In another example, the mutant soluble ActRIIB-ECD may consist of a sequence of SEQ ID NO: 219. In another example, the mutant soluble ActRIIB-ECD may consist of a sequence of SEQ ID NO: 220. In another example, the mutant soluble ActRIIB-ECD may comprise a sequence of SEQ ID NO: 221.

In some embodiments, the mutant soluble ActRIIB-ECD may comprise a sequence of any one of SEQ ID NOs: 3-37 and 51-117, in which the serine at position S20 is substituted with another amino acid that is not serine(S) or threonine (T). In one example, the mutant soluble ActRIIB-ECD may comprise a sequence of any one of SEQ ID NOs: 3-37 and 51-117, in which the serine at position S20 is substituted with an alanine. In another example, the mutant soluble ActRIIB-ECD may comprise a sequence of any one of SEQ ID NOs: 3-37 and 51-117, in which the serine at position S20 is substituted with an arginine. In another example, the mutant soluble ActRIIB-ECD may comprise a sequence of any one of SEQ ID NOs: 3-37 and 51-117, in which the serine at position S20 is substituted with an asparagine. In another example, the mutant soluble ActRIIB-ECD may comprise a sequence of any one of SEQ ID NOs: 3-37 and 51-117, in which the serine at position S20 is substituted with an aspartate. In another example, the mutant soluble ActRIIB-ECD may comprise a sequence of any one of SEQ ID NOs: 3-37 and 51-117, in which the serine at position S20 is substituted with a cysteine. In another example, the mutant soluble ActRIIB-ECD may comprise a sequence of any one of SEQ ID NOs: 3-37 and 51-117, in which the serine at position S20 is substituted with a glutamine. In another example, the mutant soluble ActRIIB-ECD may comprise a sequence of any one of SEQ ID NOs: 3-37 and 51-117, in which the serine at position S20 is substituted with a glutamate. In another example, the mutant soluble ActRIIB-ECD may comprise a sequence of any one of SEQ ID NOs: 3-37 and 51-117, in which the serine at position S20 is substituted with a glycine. In another example, the mutant soluble ActRIIB-ECD may comprise a sequence of any one of SEQ ID NOs: 3-37 and 51-117, in which the serine at position S20 is substituted with a histidine. In another example, the mutant soluble ActRIIB-ECD may comprise a sequence of any one of SEQ ID NOs: 3-37 and 51-117, in which the serine at position S20 is substituted with an isoleucine. In another example, the mutant soluble ActRIIB-ECD may comprise a sequence of any one of SEQ ID NOs: 3-37 and 51-117, in which the serine at position S20 is substituted with a leucine. In another example, the mutant soluble ActRIIB-ECD may comprise a sequence of any one of SEQ ID NOs: 3-37 and 51-117, in which the serine at position S20 is substituted with a lysine. In another example, the mutant soluble ActRIIB-ECD may comprise a sequence of any one of SEQ ID NOs: 3-37 and 51-117, in which the serine at position S20 is substituted with a methionine. In another example, the mutant soluble ActRIIB-ECD may comprise a sequence of any one of SEQ ID NOs: 3-37 and 51-117, in which the serine at position S20 is substituted with a phenylalanine. In another example, the mutant soluble ActRIIB-ECD may comprise a sequence of any one of SEQ ID NOs: 3-37 and 51-117, in which the serine at position S20 is substituted with a proline. In another example, the mutant soluble ActRIIB-ECD may comprise a sequence of any one of SEQ ID NOs: 3-37 and 51-117, in which the serine at position S20 is substituted with a tryptophan. In another example, the mutant soluble ActRIIB-ECD may comprise a sequence of any one of SEQ ID NOs: 3-37 and 51-117, in which the serine at position S20 is substituted with a tyrosine. In another example, the mutant soluble ActRIIB-ECD may comprise a sequence of any one of SEQ ID NOs: 3-37 and 51-117, in which the serine at position S20 is substituted with a valine.

In some embodiments, the one more additional mutations may be introduced so that the mutant soluble ActRIIB-ECD demonstrates decreased affinity for activin while retaining binding to Growth Differentiation Factor 11 (GDF11). Such mutants may exhibit desired effects on muscle but reduced effects on other tissues. In some examples, the additional mutation in the mutant soluble ActRIIB-ECD may be a substitution of the leucine corresponding to position L55 of SEQ ID NO: 1 with an acidic amino acid (e.g., aspartate (D) or glutamate (E)). The L55D and L55E variants may show substantial loss of activin binding while retaining almost wild type inhibition of GDF-11. Methods of measuring the effects of L55D and L55E on the binding of the mutant soluble ActRIIB-ECD include those described in WO2008097541, which is incorporated by reference herein in its entirety.

In some examples, the additional mutation in the mutant soluble ActRIIB-ECD may be a substitution of the leucine corresponding to position L55 of SEQ ID NO: 1 with an aspartate (D). In some examples, the mutant soluble ActRIIB-ECD may comprise a sequence at least 80% (e.g., at least 85%, 90%, 95%, 96%, 97%, or 98%) identical to SEQ ID NO: 327, in which the asparagine at position N18 is substituted with another amino acid. For example, the mutant soluble ActRIIB-ECD may comprise a sequence at least 95% identical to SEQ ID NO: 327, in which the asparagine at position N18 is substituted with another amino acid. In one example, the mutant soluble ActRIIB-ECD may comprise a sequence of SEQ ID NO: 328. In one example, the mutant soluble ActRIIB-ECD may consist of a sequence of SEQ ID NO: 328. In some examples, the mutant soluble ActRIIB-ECD may comprise a sequence at least 80% (e.g., at least 85%, 90%, 95%, 96%, 97%, or 98%) identical to SEQ ID NO: 327, in which the asparagine at position N18 is substituted with glutamine. For example, the mutant soluble ActRIIB-ECD may comprise a sequence at least 95% identical to SEQ ID NO: 327, in which the asparagine at position N18 is substituted with glutamine. In one example, the mutant soluble ActRIIB-ECD may comprise a sequence of SEQ ID NO: 329. In one example, the mutant soluble ActRIIB-ECD may consist of a sequence of SEQ ID NO: 329. In some examples, the mutant soluble ActRIIB-ECD may comprise a sequence at least 80% (e.g., at least 85%, 90%, 95%, 96%, 97%, or 98%) identical to SEQ ID NO: 327, in which the serine at position S20 is substituted with another amino acid that is not serine(S) or threonine (T).

In some examples, the additional mutation in the mutant soluble ActRIIB-ECD may be a substitution of the leucine corresponding to position L55 of SEQ ID NO: 1 with a glutamate (E). In some examples, the mutant soluble ActRIIB-ECD may comprise a sequence at least 80% (e.g., at least 85%, 90%, 95%, 96%, 97%, or 98%) identical to SEQ ID NO: 330 in which the asparagine at position N18 is substituted with another amino acid. For example, the mutant soluble ActRIIB-ECD may comprise a sequence at least 95% identical to SEQ ID NO: 330, in which the asparagine at position N18 is substituted with another amino acid. In one example, the mutant soluble ActRIIB-ECD may comprise a sequence of SEQ ID NO: 331. In one example, the mutant soluble ActRIIB-ECD may consist of a sequence of SEQ ID NO: 331. In some examples, the mutant soluble ActRIIB-ECD may comprise a sequence at least 80% (e.g., at least 85%, 90%, 95%, 96%, 97%, or 98%) identical to SEQ ID NO: 330, in which the asparagine at position N18 is substituted with glutamine. For example, the mutant soluble ActRIIB-ECD may comprise a sequence at least 95% identical SEQ ID NO: 330, in which the asparagine at position N18 is substituted with glutamine. In one example, the mutant soluble ActRIIB-ECD may comprise a sequence of SEQ ID NO: 332. In one example, the mutant soluble ActRIIB-ECD may consist of a sequence of SEQ ID NO: 332. In some examples, the mutant soluble ActRIIB-ECD may comprise a sequence at least 80% (e.g., at least 85%, 90%, 95%, 96%, 97%, or 98%) identical to SEQ ID NO: 330 in which the serine at position S20 is substituted with another amino acid that is that is not serine(S) or threonine (T).

In some embodiments, the additional mutations may include mutations resulting from naturally polymorphism. In some examples, in the mutant soluble ActRIIB-ECD, the arginine (R) corresponding to position R40 of SEQ ID NO: 1 may be substituted with an alanine (A). The R40A mutant may show an altered ligand binding affinity. For example, the R40A mutation may cause decreased GDF-11 and/or activin A inhibition, which may be desired in certain applications. Methods of measuring the effects on ligand binding or inhibition include those described in WO2006012627, which is incorporated by reference herein in its entirety.

In some examples, the additional mutation in the mutant soluble ActRIIB-ECD may be a substitution of the arginine corresponding to position R40 of SEQ ID NO: 1 with an alanine (A). In some examples, the mutant soluble ActRIIB-ECD may comprise a sequence at least 80% (e.g., at least 85%, 90%, 95%, 96%, 97%, or 98%) identical to SEQ ID NO: 333, in which the asparagine at position N18 is substituted with another amino acid. For example, the mutant soluble ActRIIB-ECD may comprise a sequence at least 95% identical to SEQ ID NO: 333, in which the asparagine at position N18 is substituted with another amino acid. In one example, the mutant soluble ActRIIB-ECD may comprise a sequence of SEQ ID NO: 334. In one example, the mutant soluble ActRIIB-ECD may consist of a sequence of SEQ ID NO: 334. In some examples, the mutant soluble ActRIIB-ECD may comprise a sequence at least 80% (e.g., at least 85%, 90%, 95%, 96%, 97%, or 98%) identical to SEQ ID NO: 333, in which the asparagine at position N18 is substituted with glutamine. For example, the mutant soluble ActRIIB-ECD may comprise a sequence at least 95% identical to SEQ ID NO: 333, in which the asparagine at position N18 is substituted with glutamine. In one example, the mutant soluble ActRIIB-ECD may comprise a sequence of SEQ ID NO: 335. In one example, the mutant soluble ActRIIB-ECD may consist of a sequence of SEQ ID NO: 335. In some examples, the mutant soluble ActRIIB-ECD may comprise a sequence at least 80% (e.g., at least 85%, 90%, 95%, 96%, 97%, or 98%) identical to SEQ ID NO: 333, in which the serine at position S20 is substituted with another amino acid that is that is not serine(S) or threonine (T).

In some embodiments, the isolated protein may comprise a mutant ActRIIB-ECD, which may comprise a sequence selected from the group consisting of SEQ ID NOs: 333-335, wherein the ActRIIB-ECD may further comprise from 1 to 6 additional amino acids on the N-terminus of the sequence corresponding to amino acids 1-6 of SEQ ID NO: 46. In some examples, the mutant ActRIIB-ECD may comprise a sequence of SEQ ID NO: 1613, 1626, or 1639, in which N19 is substituted with another amino acid. For example, the mutant ActRIIB-ECD may comprise a sequence of SEQ ID NO: 1619, 1632, or 1645. In some examples, the mutant ActRIIB-ECD may comprise a sequence of SEQ ID NO: 1614, 1627, or 1640, in which N20 is substituted with another amino acid. For example, the mutant ActRIIB-ECD may comprise a sequence of SEQ ID NO: 1620, 1633, or 1646. In some examples, the mutant ActRIIB-ECD may comprise a sequence of SEQ ID NO: 1615, 1628, or 1641, in which N21 is substituted with another amino acid. For example, the mutant ActRIIB-ECD may comprise a sequence of SEQ ID NO: 1621, 1634, or 1647. In some examples, the mutant ActRIIB-ECD may comprise a sequence of SEQ ID NO: 1616, 1629, or 1642, in which N22 is substituted with another amino acid. For example, the mutant ActRIIB-ECD may comprise a sequence of SEQ ID NO: 1622, 1635, or 1648. In some examples, the mutant ActRIIB-ECD may comprise a sequence of SEQ ID NO: 1617, 1630, or 1643, in which N23 is substituted with another amino acid. For example, the mutant ActRIIB-ECD may comprise a sequence of SEQ ID NO: 1623, 1636, or 1649. In some examples, the mutant ActRIIB-ECD may comprise a sequence of SEQ ID NO: 1618, 1631, or 1644, in which N24 is substituted with another amino acid. For example, the mutant ActRIIB-ECD may comprise a sequence of SEQ ID NO: 1624, 1637, or 1650.

In some examples, the mutant ActRIIB-ECD may comprise a sequence of SEQ ID NO: 1613, 1626, or 1639, in which S21 is substituted with another amino acid other than serine(S) or threonine (T). In some examples, the mutant ActRIIB-ECD may comprise a sequence of SEQ ID NO: 1614, 1627, or 1640, in which S22 is substituted with another amino acid other than serine(S) or threonine (T). In some examples, the mutant ActRIIB-ECD may comprise a sequence of SEQ ID NO: 1615, 1628, or 1641, in which S23 is substituted with another amino acid other than serine(S) or threonine (T). In some examples, the mutant ActRIIB-ECD may comprise a sequence of SEQ ID NO: 1616, 1629, or 1642, in which S24 is substituted with another amino acid other than serine(S) or threonine (T). In some examples, the mutant ActRIIB-ECD may comprise a sequence of SEQ ID NO: 1617, 1630, or 1643, in which S25 is substituted with another amino acid other than serine(S) or threonine (T). In some examples, the mutant ActRIIB-ECD may comprise a sequence of SEQ ID NO: 1618, 1631, or 1644, in which S26 is substituted with another amino acid other than serine(S) or threonine (T).

In some embodiments, the isolated protein may comprise a mutant of ActRIIB-ECD that has been developed, tested, and/or used as a therapeutic agent. For example, the isolated protein may comprise a mutant of the ActRIIB-ECD polypeptide in luspatercept (SEQ ID NO: 1658). In some embodiments, the isolated protein may comprise a mutant soluble ActRIIB-ECD comprising a mutation that removes the glycosylation site at N18 of SEQ ID NO: 1658. In some embodiments, the isolated protein may comprise the sequence of SEQ ID NO: 1664.

In some embodiments, the mutant soluble ActRIIB-ECD may comprise a mutation at the position corresponding to position N18 of SEQ ID NO: 1658 (i.e., the asparagine at position N18 is substituted with another amino acid). For example, the mutant soluble ActRIIB-ECD may comprise a sequence of SEQ ID NO: 1659.

In some examples, the mutant soluble ActRIIB-ECD may comprise a N18Q mutation, i.e., in the mutant soluble ActRIIB-ECD, the asparagine at position N18 or a position corresponding to N18 is substituted with glutamine. For example, the mutant soluble ActRIIB-ECD may comprise a sequence of SEQ ID NO: 1660. In another example, the mutant soluble ActRIIB-ECD may consist of a sequence of SEQ ID NO: 1660.

In some examples, the mutant soluble ActRIIB-ECD polypeptide may comprise a sequence at least 80% (e.g., at least 85%, 90%, 95%, 96%, 97%, or 98%) identical to SEQ ID NO: 1659, in which the asparagine at position N18 is substituted with another amino acid. For example, the mutant soluble ActRIIB-ECD polypeptide may comprise a sequence at least 95% identical to SEQ ID NO: 1659, in which the asparagine at position N18 is substituted with another amino acid.

In some examples, the mutant soluble ActRIIB-ECD polypeptide may comprise a sequence at least 80% (e.g., at least 85%, 90%, 95%, 96%, 97%, or 98%) identical to SEQ ID NO: 1660, in which the asparagine at position N18 is substituted with glutamine. For example, the mutant soluble ActRIIB-ECD polypeptide may comprise a sequence at least 95% identical to SEQ ID NO: 1660, in which the asparagine at position N18 is substituted with glutamine.

In some embodiments, the mutant soluble ActRIIB-ECD may comprise a mutation at position corresponding to position S20 of SEQ ID NO: 1658 (i.e., the asparagine at position S20 is substituted with another amino acid other than serine(S) or threonine (T)). For example, the mutant soluble ActRIIB-ECD may comprise a sequence of SEQ ID NO: 1661.

In some examples, the mutant soluble ActRIIB-ECD polypeptide may comprise a sequence at least 80% (e.g., at least 85%, 90%, 95%, 96%, 97%, or 98%) identical to SEQ ID NO: 1661, in which the asparagine at position S20 is substituted with another amino acid than serine(S) or threonine (T). For example, the mutant soluble ActRIIB-ECD polypeptide may comprise a sequence at least 95% identical to SEQ ID NO: 1661, in which the asparagine at position S20 is substituted with another amino acid than serine(S) or threonine (T).

### Binding to Partners and Modifications

In some embodiments, the mutant soluble ActRIIA-ECD or ActRIIB-ECD polypeptides demonstrate increased binding of a binding partner compared to relative to an otherwise identical soluble ActRIIB-ECD that includes the N-linked glycosylation site corresponding to position N18 of SEQ ID NO: 1. In some examples, the binding partner may be myostatin. In some examples, the binding partner may be activin. The binding of the mutant soluble ActRIIA-ECD or ActRIIB-ECD with its binding partner may be measured by any method for determining protein-protein interactions, e.g., the method described in Example 3.

In some examples, the mutant soluble ActRIIA-ECD or ActRIIB-ECD polypeptides demonstrate at least 10%, 20%, 30%, 40%, 50%, 60%, 70%, 80%, or 90% higher level of binding to a binding partner relative to an otherwise identical soluble ActRIIA-ECD or ActRIIB-ECD that includes the N-linked glycosylation site corresponding to position N18 of SEQ ID NO: 1. In some examples, the mutant soluble ActRIIB-ECD polypeptides herein demonstrate at least 10%, 20%, 30%, 40%, 50%, 60%, 70%, 80%, or 90% higher level of binding to myostatin relative to an otherwise identical soluble ActRIIA-ECD or ActRIIB-ECD that includes the N-linked glycosylation site corresponding to position N18 of SEQ ID NO: 1. In some examples, the mutant soluble ActRIIA-ECD or ActRIIB-ECD polypeptides herein demonstrate at least 10%, 20%, 30%, 40%, 50%, 60%, 70%, 80%, or 90% higher level of binding to activin relative to an otherwise identical soluble ActRIIA-ECD or ActRIIB-ECD that includes the N-linked glycosylation site corresponding to position N18 of SEQ ID NO: 1. The binding level may refer to the binding affinity, which is the measurement of the strength of the binding interaction between two molecules. The binding level between the soluble ActRIIA-ECD or ActRIIB-ECD and its binding partner may be measured by enzyme-linked immunosorbent assays (ELISA), as shown in Example 3.

In some embodiments, the removal of the N-linked glycosylation site corresponding to position N18 of SEQ ID NO: 1 does not eliminate or affect glycosylation on other site. In some examples, the mutant soluble ActRIIA-ECD or ActRIIB-ECD polypeptide may be glycosylated at an asparagine residue corresponding to positions N41 of SEQ ID NO: 1. In some examples, the mutant soluble ActRIIA-ECD or ActRIIB-ECD polypeptide may be glycosylated at an asparagine residue corresponding to positions N41 of SEQ ID NO: 1.

In some embodiments, the mutant soluble ActRIIA-ECD or ActRIIB polypeptide may comprise a certain level of sialylation. For example, a sample of the mutant soluble ActRIIB polypeptide may comprise at least 5%, 10%, 20%, 30%, 40%, 50%, 60%, 70%, 80%, or 90% sialylated glycans. In some examples, a sample of the mutant soluble ActRIIA-ECD or ActRIIB polypeptide may comprise at least 40% sialylated glycans. In some embodiments, a sample of the mutant soluble ActRIIA-ECD or ActRIIB-ECD comprises at least 5%, 10%, 20%, 30%, 40%, 50%, 60%, 70%, 80%, or 90% sialylated glycans at the position corresponding to N41 of SEQ ID NO: 1. In some preferred embodiments, a sample of the mutant soluble ActRIIA-ECD or ActRIIB-ECD comprises at least 40%, 45%, 50%, 55%, 60%, 65%, 70%, 75%, 80%, 85%, 90% or more sialylated glycans at the position corresponding to N41 of SEQ ID NO: 1. In more preferred embodiments, a sample of the mutant soluble ActRIIA-ECD or ActRIIB-ECD comprises at least 60%, 65%, 70%, 75%, 80%, 85%, 90% or more sialylated glycans at the position corresponding to N41 of SEQ ID NO: 1. In especially preferred embodiments, a sample of the mutant soluble ActRIIA-ECD or ActRIIB-ECD comprises at least 70%, 75%, 80%, 85%, 90% or more sialylated glycans at the position corresponding to N41 of SEQ ID NO: 1. The level of sialylation may be measured by any suitable method, including those described in Gerhild Zauner et al., Electrophoresis. 2011 December; 32 (24): 3456-66. doi: 10.1002/elps.201100247, which is incorporated by reference herein in its entirety. The level of sialylation can be optimized by methods known in the art. A cell line may be selected for production of higher levels of sialylation. In addition or alternatively, purification methods may be used to enrich or isolate proteins with desired level of sialylation. In one example, a sample may be fractionated (e.g., by ion exchange chromatography) to enrich for products with desired (e.g., higher) level of sialylation.

### Heterologous Proteins

In another aspect, the isolated protein according to the present disclosure may comprise a mutant soluble ActRIIA-ECD or ActRIIB-ECD polypeptide and at least one heterologous protein attached to the ActRIIA-ECD or ActRIIB-ECD polypeptide either directly or through one or more linkers to form a fusion protein.

The term “heterologous” as used herein refers to a composition or state that is not native or naturally found, for example, that may be achieved by replacing an existing natural composition or state with one that is derived from another source. Similarly the expression of a protein in an organism other than the organism in which that protein is naturally expressed constitutes a heterologous expression system and a heterologous protein. In some examples, a fusion protein comprising Protein A and a heterologous Protein B may refer to the cases where the fusion protein is not naturally found.

As used herein the term “fusion protein” refers to a protein having a heterologous polypeptide attached (e.g., via recombinant DNA techniques). Examples of the heterologous proteins include a polyhistidine tag, a Glu-Glu, a glutathione S transferase (GST), a thioredoxin, a protein A, a protein G, a fluorescent protein, a maltose binding protein (MBP), a human serum albumin or an Fc polypeptide or Fc domain.

In some embodiments, the heterologous protein comprises a constant domain of an immunoglobulin, e.g., an Fc domain of an immunoglobulin. In various embodiments, the Fc domain is a human IgG Fc domain. The Fc domain may be the Fc domain of a human immunoglobulin gamma-1 (IgG1), the Fc domain of a human immunoglobulin gamma-2 (IgG2), or the Fc domain of a human immunoglobulin gamma-4 (IgG4). In various embodiments, the Fc domain is derived from the human IgG1 heavy chain constant domain sequence set forth in SEQ ID NO: 38. In various embodiments, the Fc domain comprises the amino acid sequence set forth in SEQ ID NO: 39. In various embodiments, the Fc domain is derived from the human IgG2 heavy chain constant domain sequence set forth in SEQ ID NO: 40. In various embodiments, the Fc domain comprises the amino acid sequence set forth in SEQ ID NO: 41. In various embodiments, the Fc domain is derived from the human IgG4 heavy chain constant domain sequence set forth in SEQ ID NO: 42. In some embodiments, the Fc domain is derived from the human IgG4 heavy chain constant domain sequence set forth in SEQ ID NO: 42 and comprises an S228P mutation. In various embodiments, the Fc domain comprises the amino acid sequence set forth in SEQ ID NO: 43. In some embodiments, the Fc domain of SEQ ID NO: 39, SEQ ID NO: 41 or SEQ ID NO: 43 may further comprise a lysine (K) residue at the C-terminus of the sequence.

In some embodiments, the Fc domains include mutations to eliminate glycosylation and/or to reduce Fc-gamma receptor binding. In some embodiments, the Fc domains are human Fc domains and comprise the mutation N297Q, N297A, or N297G; in some embodiments the human Fc domains comprise a mutation at position 234 and/or 235, for example L235E, or L234A and L235A (in IgG1), or F234A and L235A (in IgG4); in some embodiments the human Fc domains are IgG2 Fc domains that comprise the mutations V234A, G237A, P238S, H268Q/A, V309L, A330S, or P331S, or a combination thereof (all according to EU numbering).

Additional examples of modified human Fc domains are known to those skilled in the art. Examples of human IgG heavy chain constant region amino acids in which mutations in at least one amino acid leads to reduced Fc function include, but are not limited to, mutations in amino acid 228, 233, 234, 235, 236, 237, 239, 252, 254, 256, 265, 270, 297, 318, 320, 322, 327, 329, 330, and 331 of the heavy constant region (according to EU numbering). Examples of combinations of mutated amino acids are also known in the art, such as, but not limited to a combination of mutations in amino acids 234, 235, and 331, such as L234F, L235E, and P331S or a combination of amino acids 318, 320, and 322, such as E318A, K320A, and K322A. In some embodiments, the Fc domain comprises one or more substitutions selected from the group consisting of N297A in IgG1, N297Q in IgG1, and S228P in IgG4.

### Linkers

In some embodiments, the isolated protein according to the present disclosure may further comprise one or more linkers between two components in the isolated protein, e.g., between the mutant soluble ActRIIA-ECD or ActRIIB-ECD polypeptide and the heterologous protein (e.g., Fc domain). For example, the one or more linkers may serve as a spacer between a mutant soluble ActRIIA-ECD or ActRIIB-ECD polypeptide and a heterologous protein or other type of fusion, or between two or more mutant soluble ActRIIB-ECD polypeptides. In some examples, the heterologous protein may be attached to the mutant soluble ActRIIA-ECD or ActRIIB-ECD polypeptide via one or more linkers.

A linker may be a peptide sequence (e.g., an artificial peptide sequence). The linker may be relatively free from secondary structure. The linker may have from 1 to 50 amino acids in length. For example, a linker may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some examples, the linker may more than 50 amino acids in length.

In various embodiments, the linkers may comprise amino acids selected from glycine, alanine, proline, asparagine, glutamine, and lysine. In various embodiments, a linker may be made up of a majority of amino acids that are sterically unhindered, such as glycine and alanine, and are polyglycines (e.g., (Gly) 5. (Gly) 8), poly(Gly-Ala), and polyalanines. In various embodiments, the linker may be rich in G/S content (e.g., at least about 60%, 70%, 80%, 90%, or more of the amino acids in the linker are G or S. In various embodiments, the linker has a (GGGGS (SEQ ID NO: 44)) n motif, wherein n=1-6. Examples of such linkers include those described extensively in art (see, e.g., U.S. Pat. No. 8,410,043 (Sun et al), incorporated by reference herein for the purposes of teaching such linkers).

In some examples, the linker may be a hinge linker, which comprises one or more amino acid residues (e.g., cysteines) capable of forming a covalent bond (hinge). When the fusion protein is multimerized (e.g., dimerized), one or more covalent bonds (e.g., disulfide bonds) may be formed between the hinge linkers on the monomers. An example of the hinge linker is a sequence of SEQ ID NO: 118.

In some examples, the isolated protein may comprise a linker comprising the amino acid sequence set forth in SEQ ID NO: 44 between the mutant ActRIIB polypeptide and the heterologous protein (e.g., Fc domain). In some examples, the isolated protein may comprise a hinge linker comprising the amino acid sequence set forth in SEQ ID NO: 118 between the mutant ActRIIB polypeptide and the heterologous protein (e.g., Fc domain). In some examples, the isolated protein may comprises a linker comprising the amino acid sequence set forth in SEQ ID NO: 44 and a hinge linker comprising the amino acid sequence set forth in SEQ ID NO: 118 between the mutant ActRIIB polypeptide and the heterologous protein (e.g., Fc domain).

In various embodiments, a linker having the amino acid sequence set forth in SEQ ID NO: 44 and a hinge linker having the amino acid sequence set forth in SEQ ID NO: 118 is used to link a human IgG1 Fc (SEQ ID NO: 39), a human IgG2 Fc (SEQ ID NO: 41), or a human IgG4 Fc (SEQ ID NO: 43) to a mutant soluble ActRIIA-ECD or ActRIIB-ECD polypeptide of the present disclosure.

Linkers may also be non-peptide linkers. For example, alkyl linkers such as —NH— (CH₂)ₛ—C(O)—, wherein s=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc.

### Isolated Proteins

It is understood that the different elements of the isolated proteins (e.g., the mutant soluble ActRIIB polypeptide, linker(s), and heterologous protein (e.g., Fc domain)) may be arranged in any manner that is consistent with the desired functionality.

For example, a heterologous protein may be attached (directly or indirectly) to the C-terminus of a mutant soluble ActRIIA-ECD or ActRIIB-ECD polypeptide. Alternatively, a heterologous protein may be attached (directly or indirectly) to the N-terminus of a mutant soluble ActRIIA-ECD or ActRIIB-ECD polypeptide. The mutant soluble ActRIIA-ECD or ActRIIB-ECD polypeptide and the heterologous domain need not be adjacent, and additional domains or amino acid sequences may be included C- or N-terminal to either domain or between the domains (e.g., include a linker described herein).

In the isolated proteins comprising the mutant soluble ActRIIA-ECD or ActRIIB-ECD and the heterologous protein (e.g., Fc domain), the removal of the N-linked glycosylation site on the mutant soluble ActRIIA-ECD or ActRIIB-ECD corresponding to position N18 of SEQ ID NO: 1 does not eliminate or affect glycosylation on other sites. For example, when the N-linked glycosylation site on the mutant soluble ActRIIA-ECD or ActRIIB-ECD corresponding to position N18 of SEQ ID NO: 1 is removed by mutation(s), the glycosylation on the amino acid residue(s) on the mutant ActRIIA-ECD or ActRIIB-ECD corresponding to N41 of SEQ ID NO: 1 and the amino acid residue(s) on the Fc domain (e.g., amino acid residue corresponding to position N67 of SEQ ID NO: 43) are not eliminated or affected.

In some embodiments, the isolated protein may be in a multimerized form. For example, the isolated protein may be in a dimerized form, e.g., forming a dimer through the heterologous protein, e.g., Fc domain. In some examples, a hinge may be formed between one or more amino acid residues of the monomers of the dimer. The one or more amino acid residues may be on the hinge linker. Alternatively or additionally, the one or more amino acid residues may be on the mutant soluble ActRIIB polypeptide and/or the heterologous protein.

The isolated protein may be a multimer comprising a plurality of monomers. In some examples, the monomers may be the same. In some examples, at least two of the monomers are different. In one example, a monomer may comprise a mutation that removes a glycosylation site (e.g., the mutations described herein) and another monomer does not comprise such mutation. In some examples, the isolated protein may be a dimer comprising two monomers. In one example, the two monomers may be the same. In another example, the two monomers may be different.

In some embodiments, the isolated protein may comprise a signal peptide. When the protein is expressed in a cell, the signal peptide may be present at the N-terminus of the protein and prompt the cell to secret the protein. Examples of signal sequences include any of SEQ ID NOs: 49 or 50. In some embodiments, the isolated protein does not have a signal peptide.

In some examples, an isolated protein may be a fusion protein comprising, in an N-terminus to C-terminus direction, mutant soluble ActRIIA-ECD or ActRIIB-ECD polypeptide, one or more linkers, and a heterologous protein (e.g., Fc domain). In some examples, an isolated protein may be a fusion protein comprising, in an N-terminus to C-terminus direction, a signal peptide, a mutant soluble ActRIIA-ECD or ActRIIB-ECD polypeptide, one or more linkers, and a heterologous protein (e.g., Fc domain).

In some examples, an isolated protein may be a fusion protein comprising, in an N-terminus to C-terminus direction, a heterologous protein (e.g., Fc domain), one or more linkers, and a mutant soluble ActRIIB polypeptide. In some examples, an isolated protein may be a fusion protein comprising, in an N-terminus to C-terminus direction, a signal peptide, a heterologous protein (e.g., Fc domain), one or more linkers, and a mutant soluble ActRIIA-ECD or ActRIIB-ECD polypeptide.

An example of the isolated protein may comprise SEQ ID NO: 222. Another example of the isolated protein may consist of SEQ ID NO: 222. Further examples of isolated proteins include those described in Tables 2-7 below. Each row of the tables describes an isolated protein comprising or consisting of the components from an N-terminus to C-terminus direction. Fusion proteins described in Tables 2-4 may further comprise a signal peptide of SEQ ID NO: 49. Fusion proteins described in Tables 5-7 may further comprise a signal peptide of SEQ ID NO: 50. Any of the fusion proteins may further comprise a C-terminal lysine on the heterologous protein of SEQ ID NO: 39, 41 or 43.

TABLE 2
Example configurations of fusion proteins
Components (from an N-terminus to C-terminus direction)

| Mutant Soluble | | Heterologous |
|---|---|---|
| ActRIIB-ECD polypeptide | Linker(s) | Protein |
| SEQ ID NO: 119 | SEQ ID NOs: 44 and 118 | SEQ ID NO: 39 |
| SEQ ID NO: 120 | SEQ ID NOs: 44 and 118 | SEQ ID NO: 39 |
| SEQ ID NO: 121 | SEQ ID NOs: 44 and 118 | SEQ ID NO: 39 |
| SEQ ID NO: 122 | SEQ ID NOs: 44 and 118 | SEQ ID NO: 39 |
| SEQ ID NO: 123 | SEQ ID NOs: 44 and 118 | SEQ ID NO: 39 |
| SEQ ID NO: 124 | SEQ ID NOs: 44 and 118 | SEQ ID NO: 39 |
| SEQ ID NO: 125 | SEQ ID NOs: 44 and 118 | SEQ ID NO: 39 |
| SEQ ID NO: 126 | SEQ ID NOs: 44 and 118 | SEQ ID NO: 39 |
| SEQ ID NO: 127 | SEQ ID NOs: 44 and 118 | SEQ ID NO: 39 |
| SEQ ID NO: 128 | SEQ ID NOs: 44 and 118 | SEQ ID NO: 39 |
| SEQ ID NO: 129 | SEQ ID NOs: 44 and 118 | SEQ ID NO: 39 |
| SEQ ID NO: 130 | SEQ ID NOs: 44 and 118 | SEQ ID NO: 39 |
| SEQ ID NO: 131 | SEQ ID NOs: 44 and 118 | SEQ ID NO: 39 |
| SEQ ID NO: 132 | SEQ ID NOs: 44 and 118 | SEQ ID NO: 39 |
| SEQ ID NO: 133 | SEQ ID NOs: 44 and 118 | SEQ ID NO: 39 |
| SEQ ID NO: 134 | SEQ ID NOs: 44 and 118 | SEQ ID NO: 39 |
| SEQ ID NO: 135 | SEQ ID NOs: 44 and 118 | SEQ ID NO: 39 |
| SEQ ID NO: 136 | SEQ ID NOs: 44 and 118 | SEQ ID NO: 39 |
| SEQ ID NO: 137 | SEQ ID NOs: 44 and 118 | SEQ ID NO: 39 |
| SEQ ID NO: 138 | SEQ ID NOs: 44 and 118 | SEQ ID NO: 39 |
| SEQ ID NO: 139 | SEQ ID NOs: 44 and 118 | SEQ ID NO: 39 |
| SEQ ID NO: 140 | SEQ ID NOs: 44 and 118 | SEQ ID NO: 39 |
| SEQ ID NO: 141 | SEQ ID NOs: 44 and 118 | SEQ ID NO: 39 |
| SEQ ID NO: 142 | SEQ ID NOs: 44 and 118 | SEQ ID NO: 39 |
| SEQ ID NO: 143 | SEQ ID NOs: 44 and 118 | SEQ ID NO: 39 |
| SEQ ID NO: 144 | SEQ ID NOs: 44 and 118 | SEQ ID NO: 39 |
| SEQ ID NO: 145 | SEQ ID NOs: 44 and 118 | SEQ ID NO: 39 |
| SEQ ID NO: 146 | SEQ ID NOs: 44 and 118 | SEQ ID NO: 39 |
| SEQ ID NO: 147 | SEQ ID NOs: 44 and 118 | SEQ ID NO: 39 |
| SEQ ID NO: 148 | SEQ ID NOs: 44 and 118 | SEQ ID NO: 39 |
| SEQ ID NO: 149 | SEQ ID NOs: 44 and 118 | SEQ ID NO: 39 |
| SEQ ID NO: 150 | SEQ ID NOs: 44 and 118 | SEQ ID NO: 39 |
| SEQ ID NO: 151 | SEQ ID NOs: 44 and 118 | SEQ ID NO: 39 |
| SEQ ID NO: 152 | SEQ ID NOs: 44 and 118 | SEQ ID NO: 39 |
| SEQ ID NO: 153 | SEQ ID NOs: 44 and 118 | SEQ ID NO: 39 |
| SEQ ID NO: 154 | SEQ ID NOs: 44 and 118 | SEQ ID NO: 39 |
| SEQ ID NO: 155 | SEQ ID NOs: 44 and 118 | SEQ ID NO: 39 |
| SEQ ID NO: 156 | SEQ ID NOs: 44 and 118 | SEQ ID NO: 39 |
| SEQ ID NO: 157 | SEQ ID NOs: 44 and 118 | SEQ ID NO: 39 |
| SEQ ID NO: 158 | SEQ ID NOs: 44 and 118 | SEQ ID NO: 39 |
| SEQ ID NO: 159 | SEQ ID NOs: 44 and 118 | SEQ ID NO: 39 |
| SEQ ID NO: 160 | SEQ ID NOs: 44 and 118 | SEQ ID NO: 39 |
| SEQ ID NO: 161 | SEQ ID NOs: 44 and 118 | SEQ ID NO: 39 |
| SEQ ID NO: 162 | SEQ ID NOs: 44 and 118 | SEQ ID NO: 39 |
| SEQ ID NO: 163 | SEQ ID NOs: 44 and 118 | SEQ ID NO: 39 |
| SEQ ID NO: 164 | SEQ ID NOs: 44 and 118 | SEQ ID NO: 39 |
| SEQ ID NO: 165 | SEQ ID NOs: 44 and 118 | SEQ ID NO: 39 |
| SEQ ID NO: 166 | SEQ ID NOs: 44 and 118 | SEQ ID NO: 39 |
| SEQ ID NO: 167 | SEQ ID NOs: 44 and 118 | SEQ ID NO: 39 |
| SEQ ID NO: 168 | SEQ ID NOs: 44 and 118 | SEQ ID NO: 39 |
| SEQ ID NO: 169 | SEQ ID NOs: 44 and 118 | SEQ ID NO: 39 |
| SEQ ID NO: 170 | SEQ ID NOs: 44 and 118 | SEQ ID NO: 39 |
| SEQ ID NO: 171 | SEQ ID NOs: 44 and 118 | SEQ ID NO: 39 |
| SEQ ID NO: 172 | SEQ ID NOs: 44 and 118 | SEQ ID NO: 39 |
| SEQ ID NO: 173 | SEQ ID NOs: 44 and 118 | SEQ ID NO: 39 |
| SEQ ID NO: 174 | SEQ ID NOs: 44 and 118 | SEQ ID NO: 39 |
| SEQ ID NO: 175 | SEQ ID NOs: 44 and 118 | SEQ ID NO: 39 |
| SEQ ID NO: 176 | SEQ ID NOs: 44 and 118 | SEQ ID NO: 39 |
| SEQ ID NO: 177 | SEQ ID NOs: 44 and 118 | SEQ ID NO: 39 |
| SEQ ID NO: 178 | SEQ ID NOs: 44 and 118 | SEQ ID NO: 39 |
| SEQ ID NO: 179 | SEQ ID NOs: 44 and 118 | SEQ ID NO: 39 |
| SEQ ID NO: 180 | SEQ ID NOs: 44 and 118 | SEQ ID NO: 39 |
| SEQ ID NO: 181 | SEQ ID NOs: 44 and 118 | SEQ ID NO: 39 |
| SEQ ID NO: 182 | SEQ ID NOs: 44 and 118 | SEQ ID NO: 39 |
| SEQ ID NO: 183 | SEQ ID NOs: 44 and 118 | SEQ ID NO: 39 |
| SEQ ID NO: 184 | SEQ ID NOs: 44 and 118 | SEQ ID NO: 39 |
| SEQ ID NO: 185 | SEQ ID NOs: 44 and 118 | SEQ ID NO: 39 |
| SEQ ID NO: 186 | SEQ ID NOs: 44 and 118 | SEQ ID NO: 39 |
| SEQ ID NO: 187 | SEQ ID NOs: 44 and 118 | SEQ ID NO: 39 |
| SEQ ID NO: 188 | SEQ ID NOs: 44 and 118 | SEQ ID NO: 39 |
| SEQ ID NO: 189 | SEQ ID NOs: 44 and 118 | SEQ ID NO: 39 |
| SEQ ID NO: 190 | SEQ ID NOs: 44 and 118 | SEQ ID NO: 39 |
| SEQ ID NO: 191 | SEQ ID NOs: 44 and 118 | SEQ ID NO: 39 |
| SEQ ID NO: 192 | SEQ ID NOs: 44 and 118 | SEQ ID NO: 39 |
| SEQ ID NO: 193 | SEQ ID NOs: 44 and 118 | SEQ ID NO: 39 |
| SEQ ID NO: 194 | SEQ ID NOs: 44 and 118 | SEQ ID NO: 39 |
| SEQ ID NO: 195 | SEQ ID NOs: 44 and 118 | SEQ ID NO: 39 |
| SEQ ID NO: 196 | SEQ ID NOs: 44 and 118 | SEQ ID NO: 39 |
| SEQ ID NO: 197 | SEQ ID NOs: 44 and 118 | SEQ ID NO: 39 |
| SEQ ID NO: 198 | SEQ ID NOs: 44 and 118 | SEQ ID NO: 39 |
| SEQ ID NO: 199 | SEQ ID NOs: 44 and 118 | SEQ ID NO: 39 |
| SEQ ID NO: 200 | SEQ ID NOs: 44 and 118 | SEQ ID NO: 39 |
| SEQ ID NO: 201 | SEQ ID NOs: 44 and 118 | SEQ ID NO: 39 |
| SEQ ID NO: 202 | SEQ ID NOs: 44 and 118 | SEQ ID NO: 39 |
| SEQ ID NO: 203 | SEQ ID NOs: 44 and 118 | SEQ ID NO: 39 |
| SEQ ID NO: 204 | SEQ ID NOs: 44 and 118 | SEQ ID NO: 39 |
| SEQ ID NO: 205 | SEQ ID NOs: 44 and 118 | SEQ ID NO: 39 |
| SEQ ID NO: 206 | SEQ ID NOs: 44 and 118 | SEQ ID NO: 39 |
| SEQ ID NO: 207 | SEQ ID NOs: 44 and 118 | SEQ ID NO: 39 |
| SEQ ID NO: 208 | SEQ ID NOs: 44 and 118 | SEQ ID NO: 39 |
| SEQ ID NO: 209 | SEQ ID NOs: 44 and 118 | SEQ ID NO: 39 |
| SEQ ID NO: 210 | SEQ ID NOs: 44 and 118 | SEQ ID NO: 39 |
| SEQ ID NO: 211 | SEQ ID NOs: 44 and 118 | SEQ ID NO: 39 |
| SEQ ID NO: 212 | SEQ ID NOs: 44 and 118 | SEQ ID NO: 39 |
| SEQ ID NO: 213 | SEQ ID NOs: 44 and 118 | SEQ ID NO: 39 |
| SEQ ID NO: 214 | SEQ ID NOs: 44 and 118 | SEQ ID NO: 39 |
| SEQ ID NO: 215 | SEQ ID NOs: 44 and 118 | SEQ ID NO: 39 |
| SEQ ID NO: 216 | SEQ ID NOs: 44 and 118 | SEQ ID NO: 39 |
| SEQ ID NO: 217 | SEQ ID NOs: 44 and 118 | SEQ ID NO: 39 |
| SEQ ID NO: 218 | SEQ ID NOs: 44 and 118 | SEQ ID NO: 39 |
| SEQ ID NO: 219 | SEQ ID NOs: 44 and 118 | SEQ ID NO: 39 |
| SEQ ID NO: 220 | SEQ ID NOs: 44 and 118 | SEQ ID NO: 39 |
| SEQ ID NO: 221 | SEQ ID NOs: 44 and 118 | SEQ ID NO: 39 |
| SEQ ID NO: 329 | SEQ ID NOs: 44 and 118 | SEQ ID NO: 39 |
| SEQ ID NO: 332 | SEQ ID NOs: 44 and 118 | SEQ ID NO: 39 |
| SEQ ID NO: 335 | SEQ ID NOs: 44 and 118 | SEQ ID NO: 39 |
| SEQ ID NO: 119 | SEQ ID NOs: 44 and 118 | SEQ ID NO: 41 |
| SEQ ID NO: 120 | SEQ ID NOs: 44 and 118 | SEQ ID NO: 41 |
| SEQ ID NO: 121 | SEQ ID NOs: 44 and 118 | SEQ ID NO: 41 |
| SEQ ID NO: 122 | SEQ ID NOs: 44 and 118 | SEQ ID NO: 41 |
| SEQ ID NO: 123 | SEQ ID NOs: 44 and 118 | SEQ ID NO: 41 |
| SEQ ID NO: 124 | SEQ ID NOs: 44 and 118 | SEQ ID NO: 41 |
| SEQ ID NO: 125 | SEQ ID NOs: 44 and 118 | SEQ ID NO: 41 |
| SEQ ID NO: 126 | SEQ ID NOs: 44 and 118 | SEQ ID NO: 41 |
| SEQ ID NO: 127 | SEQ ID NOs: 44 and 118 | SEQ ID NO: 41 |
| SEQ ID NO: 128 | SEQ ID NOs: 44 and 118 | SEQ ID NO: 41 |
| SEQ ID NO: 129 | SEQ ID NOs: 44 and 118 | SEQ ID NO: 41 |
| SEQ ID NO: 130 | SEQ ID NOs: 44 and 118 | SEQ ID NO: 41 |
| SEQ ID NO: 131 | SEQ ID NOs: 44 and 118 | SEQ ID NO: 41 |
| SEQ ID NO: 132 | SEQ ID NOs: 44 and 118 | SEQ ID NO: 41 |
| SEQ ID NO: 133 | SEQ ID NOs: 44 and 118 | SEQ ID NO: 41 |
| SEQ ID NO: 134 | SEQ ID NOs: 44 and 118 | SEQ ID NO: 41 |
| SEQ ID NO: 135 | SEQ ID NOs: 44 and 118 | SEQ ID NO: 41 |
| SEQ ID NO: 136 | SEQ ID NOs: 44 and 118 | SEQ ID NO: 41 |
| SEQ ID NO: 137 | SEQ ID NOs: 44 and 118 | SEQ ID NO: 41 |
| SEQ ID NO: 138 | SEQ ID NOs: 44 and 118 | SEQ ID NO: 41 |
| SEQ ID NO: 139 | SEQ ID NOs: 44 and 118 | SEQ ID NO: 41 |
| SEQ ID NO: 140 | SEQ ID NOs: 44 and 118 | SEQ ID NO: 41 |
| SEQ ID NO: 141 | SEQ ID NOs: 44 and 118 | SEQ ID NO: 41 |
| SEQ ID NO: 142 | SEQ ID NOs: 44 and 118 | SEQ ID NO: 41 |
| SEQ ID NO: 143 | SEQ ID NOs: 44 and 118 | SEQ ID NO: 41 |
| SEQ ID NO: 144 | SEQ ID NOs: 44 and 118 | SEQ ID NO: 41 |
| SEQ ID NO: 145 | SEQ ID NOs: 44 and 118 | SEQ ID NO: 41 |
| SEQ ID NO: 146 | SEQ ID NOs: 44 and 118 | SEQ ID NO: 41 |
| SEQ ID NO: 147 | SEQ ID NOs: 44 and 118 | SEQ ID NO: 41 |
| SEQ ID NO: 148 | SEQ ID NOs: 44 and 118 | SEQ ID NO: 41 |
| SEQ ID NO: 149 | SEQ ID NOs: 44 and 118 | SEQ ID NO: 41 |
| SEQ ID NO: 150 | SEQ ID NOs: 44 and 118 | SEQ ID NO: 41 |
| SEQ ID NO: 151 | SEQ ID NOs: 44 and 118 | SEQ ID NO: 41 |
| SEQ ID NO: 152 | SEQ ID NOs: 44 and 118 | SEQ ID NO: 41 |
| SEQ ID NO: 153 | SEQ ID NOs: 44 and 118 | SEQ ID NO: 41 |
| SEQ ID NO: 154 | SEQ ID NOs: 44 and 118 | SEQ ID NO: 41 |
| SEQ ID NO: 155 | SEQ ID NOs: 44 and 118 | SEQ ID NO: 41 |
| SEQ ID NO: 156 | SEQ ID NOs: 44 and 118 | SEQ ID NO: 41 |
| SEQ ID NO: 157 | SEQ ID NOs: 44 and 118 | SEQ ID NO: 41 |
| SEQ ID NO: 158 | SEQ ID NOs: 44 and 118 | SEQ ID NO: 41 |
| SEQ ID NO: 159 | SEQ ID NOs: 44 and 118 | SEQ ID NO: 41 |
| SEQ ID NO: 160 | SEQ ID NOs: 44 and 118 | SEQ ID NO: 41 |
| SEQ ID NO: 161 | SEQ ID NOs: 44 and 118 | SEQ ID NO: 41 |
| SEQ ID NO: 162 | SEQ ID NOs: 44 and 118 | SEQ ID NO: 41 |
| SEQ ID NO: 163 | SEQ ID NOs: 44 and 118 | SEQ ID NO: 41 |
| SEQ ID NO: 164 | SEQ ID NOs: 44 and 118 | SEQ ID NO: 41 |
| SEQ ID NO: 165 | SEQ ID NOs: 44 and 118 | SEQ ID NO: 41 |
| SEQ ID NO: 166 | SEQ ID NOs: 44 and 118 | SEQ ID NO: 41 |
| SEQ ID NO: 167 | SEQ ID NOs: 44 and 118 | SEQ ID NO: 41 |
| SEQ ID NO: 168 | SEQ ID NOs: 44 and 118 | SEQ ID NO: 41 |
| SEQ ID NO: 169 | SEQ ID NOs: 44 and 118 | SEQ ID NO: 41 |
| SEQ ID NO: 170 | SEQ ID NOs: 44 and 118 | SEQ ID NO: 41 |
| SEQ ID NO: 171 | SEQ ID NOs: 44 and 118 | SEQ ID NO: 41 |
| SEQ ID NO: 172 | SEQ ID NOs: 44 and 118 | SEQ ID NO: 41 |
| SEQ ID NO: 173 | SEQ ID NOs: 44 and 118 | SEQ ID NO: 41 |
| SEQ ID NO: 174 | SEQ ID NOs: 44 and 118 | SEQ ID NO: 41 |
| SEQ ID NO: 175 | SEQ ID NOs: 44 and 118 | SEQ ID NO: 41 |
| SEQ ID NO: 176 | SEQ ID NOs: 44 and 118 | SEQ ID NO: 41 |
| SEQ ID NO: 177 | SEQ ID NOs: 44 and 118 | SEQ ID NO: 41 |
| SEQ ID NO: 178 | SEQ ID NOs: 44 and 118 | SEQ ID NO: 41 |
| SEQ ID NO: 179 | SEQ ID NOs: 44 and 118 | SEQ ID NO: 41 |
| SEQ ID NO: 180 | SEQ ID NOs: 44 and 118 | SEQ ID NO: 41 |
| SEQ ID NO: 181 | SEQ ID NOs: 44 and 118 | SEQ ID NO: 41 |
| SEQ ID NO: 182 | SEQ ID NOs: 44 and 118 | SEQ ID NO: 41 |
| SEQ ID NO: 183 | SEQ ID NOs: 44 and 118 | SEQ ID NO: 41 |
| SEQ ID NO: 184 | SEQ ID NOs: 44 and 118 | SEQ ID NO: 41 |
| SEQ ID NO: 185 | SEQ ID NOs: 44 and 118 | SEQ ID NO: 41 |
| SEQ ID NO: 186 | SEQ ID NOs: 44 and 118 | SEQ ID NO: 41 |
| SEQ ID NO: 187 | SEQ ID NOs: 44 and 118 | SEQ ID NO: 41 |
| SEQ ID NO: 188 | SEQ ID NOs: 44 and 118 | SEQ ID NO: 41 |
| SEQ ID NO: 189 | SEQ ID NOs: 44 and 118 | SEQ ID NO: 41 |
| SEQ ID NO: 190 | SEQ ID NOs: 44 and 118 | SEQ ID NO: 41 |
| SEQ ID NO: 191 | SEQ ID NOs: 44 and 118 | SEQ ID NO: 41 |
| SEQ ID NO: 192 | SEQ ID NOs: 44 and 118 | SEQ ID NO: 41 |
| SEQ ID NO: 193 | SEQ ID NOs: 44 and 118 | SEQ ID NO: 41 |
| SEQ ID NO: 194 | SEQ ID NOs: 44 and 118 | SEQ ID NO: 41 |
| SEQ ID NO: 195 | SEQ ID NOs: 44 and 118 | SEQ ID NO: 41 |
| SEQ ID NO: 196 | SEQ ID NOs: 44 and 118 | SEQ ID NO: 41 |
| SEQ ID NO: 197 | SEQ ID NOs: 44 and 118 | SEQ ID NO: 41 |
| SEQ ID NO: 198 | SEQ ID NOs: 44 and 118 | SEQ ID NO: 41 |
| SEQ ID NO: 199 | SEQ ID NOs: 44 and 118 | SEQ ID NO: 41 |
| SEQ ID NO: 200 | SEQ ID NOs: 44 and 118 | SEQ ID NO: 41 |
| SEQ ID NO: 201 | SEQ ID NOs: 44 and 118 | SEQ ID NO: 41 |
| SEQ ID NO: 202 | SEQ ID NOs: 44 and 118 | SEQ ID NO: 41 |
| SEQ ID NO: 203 | SEQ ID NOs: 44 and 118 | SEQ ID NO: 41 |
| SEQ ID NO: 204 | SEQ ID NOs: 44 and 118 | SEQ ID NO: 41 |
| SEQ ID NO: 205 | SEQ ID NOs: 44 and 118 | SEQ ID NO: 41 |
| SEQ ID NO: 206 | SEQ ID NOs: 44 and 118 | SEQ ID NO: 41 |
| SEQ ID NO: 207 | SEQ ID NOs: 44 and 118 | SEQ ID NO: 41 |
| SEQ ID NO: 208 | SEQ ID NOs: 44 and 118 | SEQ ID NO: 41 |
| SEQ ID NO: 209 | SEQ ID NOs: 44 and 118 | SEQ ID NO: 41 |
| SEQ ID NO: 210 | SEQ ID NOs: 44 and 118 | SEQ ID NO: 41 |
| SEQ ID NO: 211 | SEQ ID NOs: 44 and 118 | SEQ ID NO: 41 |
| SEQ ID NO: 212 | SEQ ID NOs: 44 and 118 | SEQ ID NO: 41 |
| SEQ ID NO: 213 | SEQ ID NOs: 44 and 118 | SEQ ID NO: 41 |
| SEQ ID NO: 214 | SEQ ID NOs: 44 and 118 | SEQ ID NO: 41 |
| SEQ ID NO: 215 | SEQ ID NOs: 44 and 118 | SEQ ID NO: 41 |
| SEQ ID NO: 216 | SEQ ID NOs: 44 and 118 | SEQ ID NO: 41 |
| SEQ ID NO: 217 | SEQ ID NOs: 44 and 118 | SEQ ID NO: 41 |
| SEQ ID NO: 218 | SEQ ID NOs: 44 and 118 | SEQ ID NO: 41 |
| SEQ ID NO: 219 | SEQ ID NOs: 44 and 118 | SEQ ID NO: 41 |
| SEQ ID NO: 220 | SEQ ID NOs: 44 and 118 | SEQ ID NO: 41 |
| SEQ ID NO: 221 | SEQ ID NOs: 44 and 118 | SEQ ID NO: 41 |
| SEQ ID NO: 329 | SEQ ID NOs: 44 and 118 | SEQ ID NO: 41 |
| SEQ ID NO: 332 | SEQ ID NOs: 44 and 118 | SEQ ID NO: 41 |
| SEQ ID NO: 335 | SEQ ID NOs: 44 and 118 | SEQ ID NO: 41 |
| SEQ ID NO: 119 | SEQ ID NOs: 44 and 118 | SEQ ID NO: 41 |
| SEQ ID NO: 120 | SEQ ID NOs: 44 and 118 | SEQ ID NO: 43 |
| SEQ ID NO: 121 | SEQ ID NOs: 44 and 118 | SEQ ID NO: 43 |
| SEQ ID NO: 122 | SEQ ID NOs: 44 and 118 | SEQ ID NO: 43 |
| SEQ ID NO: 123 | SEQ ID NOs: 44 and 118 | SEQ ID NO: 43 |
| SEQ ID NO: 124 | SEQ ID NOs: 44 and 118 | SEQ ID NO: 43 |
| SEQ ID NO: 125 | SEQ ID NOs: 44 and 118 | SEQ ID NO: 43 |
| SEQ ID NO: 126 | SEQ ID NOs: 44 and 118 | SEQ ID NO: 43 |
| SEQ ID NO: 127 | SEQ ID NOs: 44 and 118 | SEQ ID NO: 43 |
| SEQ ID NO: 128 | SEQ ID NOs: 44 and 118 | SEQ ID NO: 43 |
| SEQ ID NO: 129 | SEQ ID NOs: 44 and 118 | SEQ ID NO: 43 |
| SEQ ID NO: 130 | SEQ ID NOs: 44 and 118 | SEQ ID NO: 43 |
| SEQ ID NO: 131 | SEQ ID NOs: 44 and 118 | SEQ ID NO: 43 |
| SEQ ID NO: 132 | SEQ ID NOs: 44 and 118 | SEQ ID NO: 43 |
| SEQ ID NO: 133 | SEQ ID NOs: 44 and 118 | SEQ ID NO: 43 |
| SEQ ID NO: 134 | SEQ ID NOs: 44 and 118 | SEQ ID NO: 43 |
| SEQ ID NO: 135 | SEQ ID NOs: 44 and 118 | SEQ ID NO: 43 |
| SEQ ID NO: 136 | SEQ ID NOs: 44 and 118 | SEQ ID NO: 43 |
| SEQ ID NO: 137 | SEQ ID NOs: 44 and 118 | SEQ ID NO: 43 |
| SEQ ID NO: 138 | SEQ ID NOs: 44 and 118 | SEQ ID NO: 43 |
| SEQ ID NO: 139 | SEQ ID NOs: 44 and 118 | SEQ ID NO: 43 |
| SEQ ID NO: 140 | SEQ ID NOs: 44 and 118 | SEQ ID NO: 43 |
| SEQ ID NO: 141 | SEQ ID NOs: 44 and 118 | SEQ ID NO: 43 |
| SEQ ID NO: 142 | SEQ ID NOs: 44 and 118 | SEQ ID NO: 43 |
| SEQ ID NO: 143 | SEQ ID NOs: 44 and 118 | SEQ ID NO: 43 |
| SEQ ID NO: 144 | SEQ ID NOs: 44 and 118 | SEQ ID NO: 43 |
| SEQ ID NO: 145 | SEQ ID NOs: 44 and 118 | SEQ ID NO: 43 |
| SEQ ID NO: 146 | SEQ ID NOs: 44 and 118 | SEQ ID NO: 43 |
| SEQ ID NO: 147 | SEQ ID NOs: 44 and 118 | SEQ ID NO: 43 |
| SEQ ID NO: 148 | SEQ ID NOs: 44 and 118 | SEQ ID NO: 43 |
| SEQ ID NO: 149 | SEQ ID NOs: 44 and 118 | SEQ ID NO: 43 |
| SEQ ID NO: 150 | SEQ ID NOs: 44 and 118 | SEQ ID NO: 43 |
| SEQ ID NO: 151 | SEQ ID NOs: 44 and 118 | SEQ ID NO: 43 |
| SEQ ID NO: 152 | SEQ ID NOs: 44 and 118 | SEQ ID NO: 43 |
| SEQ ID NO: 153 | SEQ ID NOs: 44 and 118 | SEQ ID NO: 43 |
| SEQ ID NO: 154 | SEQ ID NOs: 44 and 118 | SEQ ID NO: 43 |
| SEQ ID NO: 155 | SEQ ID NOs: 44 and 118 | SEQ ID NO: 43 |
| SEQ ID NO: 156 | SEQ ID NOs: 44 and 118 | SEQ ID NO: 43 |
| SEQ ID NO: 157 | SEQ ID NOs: 44 and 118 | SEQ ID NO: 43 |
| SEQ ID NO: 158 | SEQ ID NOs: 44 and 118 | SEQ ID NO: 43 |
| SEQ ID NO: 159 | SEQ ID NOs: 44 and 118 | SEQ ID NO: 43 |
| SEQ ID NO: 160 | SEQ ID NOs: 44 and 118 | SEQ ID NO: 43 |
| SEQ ID NO: 161 | SEQ ID NOs: 44 and 118 | SEQ ID NO: 43 |
| SEQ ID NO: 162 | SEQ ID NOs: 44 and 118 | SEQ ID NO: 43 |
| SEQ ID NO: 163 | SEQ ID NOs: 44 and 118 | SEQ ID NO: 43 |
| SEQ ID NO: 164 | SEQ ID NOs: 44 and 118 | SEQ ID NO: 43 |
| SEQ ID NO: 165 | SEQ ID NOs: 44 and 118 | SEQ ID NO: 43 |
| SEQ ID NO: 166 | SEQ ID NOs: 44 and 118 | SEQ ID NO: 43 |
| SEQ ID NO: 167 | SEQ ID NOs: 44 and 118 | SEQ ID NO: 43 |
| SEQ ID NO: 168 | SEQ ID NOs: 44 and 118 | SEQ ID NO: 43 |
| SEQ ID NO: 169 | SEQ ID NOs: 44 and 118 | SEQ ID NO: 43 |
| SEQ ID NO: 170 | SEQ ID NOs: 44 and 118 | SEQ ID NO: 43 |
| SEQ ID NO: 171 | SEQ ID NOs: 44 and 118 | SEQ ID NO: 43 |
| SEQ ID NO: 172 | SEQ ID NOs: 44 and 118 | SEQ ID NO: 43 |
| SEQ ID NO: 173 | SEQ ID NOs: 44 and 118 | SEQ ID NO: 43 |
| SEQ ID NO: 174 | SEQ ID NOs: 44 and 118 | SEQ ID NO: 43 |
| SEQ ID NO: 175 | SEQ ID NOs: 44 and 118 | SEQ ID NO: 43 |
| SEQ ID NO: 176 | SEQ ID NOs: 44 and 118 | SEQ ID NO: 43 |
| SEQ ID NO: 177 | SEQ ID NOs: 44 and 118 | SEQ ID NO: 43 |
| SEQ ID NO: 178 | SEQ ID NOs: 44 and 118 | SEQ ID NO: 43 |
| SEQ ID NO: 179 | SEQ ID NOs: 44 and 118 | SEQ ID NO: 43 |
| SEQ ID NO: 180 | SEQ ID NOs: 44 and 118 | SEQ ID NO: 43 |
| SEQ ID NO: 181 | SEQ ID NOs: 44 and 118 | SEQ ID NO: 43 |
| SEQ ID NO: 182 | SEQ ID NOs: 44 and 118 | SEQ ID NO: 43 |
| SEQ ID NO: 183 | SEQ ID NOs: 44 and 118 | SEQ ID NO: 43 |
| SEQ ID NO: 184 | SEQ ID NOs: 44 and 118 | SEQ ID NO: 43 |
| SEQ ID NO: 185 | SEQ ID NOs: 44 and 118 | SEQ ID NO: 43 |
| SEQ ID NO: 186 | SEQ ID NOs: 44 and 118 | SEQ ID NO: 43 |
| SEQ ID NO: 187 | SEQ ID NOs: 44 and 118 | SEQ ID NO: 43 |
| SEQ ID NO: 188 | SEQ ID NOs: 44 and 118 | SEQ ID NO: 43 |
| SEQ ID NO: 189 | SEQ ID NOs: 44 and 118 | SEQ ID NO: 43 |
| SEQ ID NO: 190 | SEQ ID NOs: 44 and 118 | SEQ ID NO: 43 |
| SEQ ID NO: 191 | SEQ ID NOs: 44 and 118 | SEQ ID NO: 43 |
| SEQ ID NO: 192 | SEQ ID NOs: 44 and 118 | SEQ ID NO: 43 |
| SEQ ID NO: 193 | SEQ ID NOs: 44 and 118 | SEQ ID NO: 43 |
| SEQ ID NO: 194 | SEQ ID NOs: 44 and 118 | SEQ ID NO: 43 |
| SEQ ID NO: 195 | SEQ ID NOs: 44 and 118 | SEQ ID NO: 43 |
| SEQ ID NO: 196 | SEQ ID NOs: 44 and 118 | SEQ ID NO: 43 |
| SEQ ID NO: 197 | SEQ ID NOs: 44 and 118 | SEQ ID NO: 43 |
| SEQ ID NO: 198 | SEQ ID NOs: 44 and 118 | SEQ ID NO: 43 |
| SEQ ID NO: 199 | SEQ ID NOs: 44 and 118 | SEQ ID NO: 43 |
| SEQ ID NO: 200 | SEQ ID NOs: 44 and 118 | SEQ ID NO: 43 |
| SEQ ID NO: 201 | SEQ ID NOs: 44 and 118 | SEQ ID NO: 43 |
| SEQ ID NO: 202 | SEQ ID NOs: 44 and 118 | SEQ ID NO: 43 |
| SEQ ID NO: 203 | SEQ ID NOs: 44 and 118 | SEQ ID NO: 43 |
| SEQ ID NO: 204 | SEQ ID NOs: 44 and 118 | SEQ ID NO: 43 |
| SEQ ID NO: 205 | SEQ ID NOs: 44 and 118 | SEQ ID NO: 43 |
| SEQ ID NO: 206 | SEQ ID NOs: 44 and 118 | SEQ ID NO: 43 |
| SEQ ID NO: 207 | SEQ ID NOs: 44 and 118 | SEQ ID NO: 43 |
| SEQ ID NO: 208 | SEQ ID NOs: 44 and 118 | SEQ ID NO: 43 |
| SEQ ID NO: 209 | SEQ ID NOs: 44 and 118 | SEQ ID NO: 43 |
| SEQ ID NO: 210 | SEQ ID NOs: 44 and 118 | SEQ ID NO: 43 |
| SEQ ID NO: 211 | SEQ ID NOs: 44 and 118 | SEQ ID NO: 43 |
| SEQ ID NO: 212 | SEQ ID NOs: 44 and 118 | SEQ ID NO: 43 |
| SEQ ID NO: 213 | SEQ ID NOs: 44 and 118 | SEQ ID NO: 43 |
| SEQ ID NO: 214 | SEQ ID NOs: 44 and 118 | SEQ ID NO: 43 |
| SEQ ID NO: 215 | SEQ ID NOs: 44 and 118 | SEQ ID NO: 43 |
| SEQ ID NO: 216 | SEQ ID NOs: 44 and 118 | SEQ ID NO: 43 |
| SEQ ID NO: 217 | SEQ ID NOs: 44 and 118 | SEQ ID NO: 43 |
| SEQ ID NO: 218 | SEQ ID NOs: 44 and 118 | SEQ ID NO: 43 |
| SEQ ID NO: 219 | SEQ ID NOs: 44 and 118 | SEQ ID NO: 43 |
| SEQ ID NO: 220 | SEQ ID NOs: 44 and 118 | SEQ ID NO: 43 |
| SEQ ID NO: 221 | SEQ ID NOs: 44 and 118 | SEQ ID NO: 43 |
| SEQ ID NO: 329 | SEQ ID NOs: 44 and 118 | SEQ ID NO: 43 |
| SEQ ID NO: 332 | SEQ ID NOs: 44 and 118 | SEQ ID NO: 43 |
| SEQ ID NO: 335 | SEQ ID NOs: 44 and 118 | SEQ ID NO: 43 |

TABLE 3
Example configurations of fusion proteins
Components (from an N-terminus to C-terminus direction)

| Mutant Soluble | | |
|---|---|---|
| ActRIIB-ECD polypeptide | Linker(s) | Heterologous Protein |
| SEQ ID NO: 119 | SEQ ID NO: 44 | SEQ ID NO: 39 |
| SEQ ID NO: 120 | SEQ ID NO: 44 | SEQ ID NO: 39 |
| SEQ ID NO: 121 | SEQ ID NO: 44 | SEQ ID NO: 39 |
| SEQ ID NO: 122 | SEQ ID NO: 44 | SEQ ID NO: 39 |
| SEQ ID NO: 123 | SEQ ID NO: 44 | SEQ ID NO: 39 |
| SEQ ID NO: 124 | SEQ ID NO: 44 | SEQ ID NO: 39 |
| SEQ ID NO: 125 | SEQ ID NO: 44 | SEQ ID NO: 39 |
| SEQ ID NO: 126 | SEQ ID NO: 44 | SEQ ID NO: 39 |
| SEQ ID NO: 127 | SEQ ID NO: 44 | SEQ ID NO: 39 |
| SEQ ID NO: 128 | SEQ ID NO: 44 | SEQ ID NO: 39 |
| SEQ ID NO: 129 | SEQ ID NO: 44 | SEQ ID NO: 39 |
| SEQ ID NO: 130 | SEQ ID NO: 44 | SEQ ID NO: 39 |
| SEQ ID NO: 131 | SEQ ID NO: 44 | SEQ ID NO: 39 |
| SEQ ID NO: 132 | SEQ ID NO: 44 | SEQ ID NO: 39 |
| SEQ ID NO: 133 | SEQ ID NO: 44 | SEQ ID NO: 39 |
| SEQ ID NO: 134 | SEQ ID NO: 44 | SEQ ID NO: 39 |
| SEQ ID NO: 135 | SEQ ID NO: 44 | SEQ ID NO: 39 |
| SEQ ID NO: 136 | SEQ ID NO: 44 | SEQ ID NO: 39 |
| SEQ ID NO: 137 | SEQ ID NO: 44 | SEQ ID NO: 39 |
| SEQ ID NO: 138 | SEQ ID NO: 44 | SEQ ID NO: 39 |
| SEQ ID NO: 139 | SEQ ID NO: 44 | SEQ ID NO: 39 |
| SEQ ID NO: 140 | SEQ ID NO: 44 | SEQ ID NO: 39 |
| SEQ ID NO: 141 | SEQ ID NO: 44 | SEQ ID NO: 39 |
| SEQ ID NO: 142 | SEQ ID NO: 44 | SEQ ID NO: 39 |
| SEQ ID NO: 143 | SEQ ID NO: 44 | SEQ ID NO: 39 |
| SEQ ID NO: 144 | SEQ ID NO: 44 | SEQ ID NO: 39 |
| SEQ ID NO: 145 | SEQ ID NO: 44 | SEQ ID NO: 39 |
| SEQ ID NO: 146 | SEQ ID NO: 44 | SEQ ID NO: 39 |
| SEQ ID NO: 147 | SEQ ID NO: 44 | SEQ ID NO: 39 |
| SEQ ID NO: 148 | SEQ ID NO: 44 | SEQ ID NO: 39 |
| SEQ ID NO: 149 | SEQ ID NO: 44 | SEQ ID NO: 39 |
| SEQ ID NO: 150 | SEQ ID NO: 44 | SEQ ID NO: 39 |
| SEQ ID NO: 151 | SEQ ID NO: 44 | SEQ ID NO: 39 |
| SEQ ID NO: 152 | SEQ ID NO: 44 | SEQ ID NO: 39 |
| SEQ ID NO: 153 | SEQ ID NO: 44 | SEQ ID NO: 39 |
| SEQ ID NO: 154 | SEQ ID NO: 44 | SEQ ID NO: 39 |
| SEQ ID NO: 155 | SEQ ID NO: 44 | SEQ ID NO: 39 |
| SEQ ID NO: 156 | SEQ ID NO: 44 | SEQ ID NO: 39 |
| SEQ ID NO: 157 | SEQ ID NO: 44 | SEQ ID NO: 39 |
| SEQ ID NO: 158 | SEQ ID NO: 44 | SEQ ID NO: 39 |
| SEQ ID NO: 159 | SEQ ID NO: 44 | SEQ ID NO: 39 |
| SEQ ID NO: 160 | SEQ ID NO: 44 | SEQ ID NO: 39 |
| SEQ ID NO: 161 | SEQ ID NO: 44 | SEQ ID NO: 39 |
| SEQ ID NO: 162 | SEQ ID NO: 44 | SEQ ID NO: 39 |
| SEQ ID NO: 163 | SEQ ID NO: 44 | SEQ ID NO: 39 |
| SEQ ID NO: 164 | SEQ ID NO: 44 | SEQ ID NO: 39 |
| SEQ ID NO: 165 | SEQ ID NO: 44 | SEQ ID NO: 39 |
| SEQ ID NO: 166 | SEQ ID NO: 44 | SEQ ID NO: 39 |
| SEQ ID NO: 167 | SEQ ID NO: 44 | SEQ ID NO: 39 |
| SEQ ID NO: 168 | SEQ ID NO: 44 | SEQ ID NO: 39 |
| SEQ ID NO: 169 | SEQ ID NO: 44 | SEQ ID NO: 39 |
| SEQ ID NO: 170 | SEQ ID NO: 44 | SEQ ID NO: 39 |
| SEQ ID NO: 171 | SEQ ID NO: 44 | SEQ ID NO: 39 |
| SEQ ID NO: 172 | SEQ ID NO: 44 | SEQ ID NO: 39 |
| SEQ ID NO: 173 | SEQ ID NO: 44 | SEQ ID NO: 39 |
| SEQ ID NO: 174 | SEQ ID NO: 44 | SEQ ID NO: 39 |
| SEQ ID NO: 175 | SEQ ID NO: 44 | SEQ ID NO: 39 |
| SEQ ID NO: 176 | SEQ ID NO: 44 | SEQ ID NO: 39 |
| SEQ ID NO: 177 | SEQ ID NO: 44 | SEQ ID NO: 39 |
| SEQ ID NO: 178 | SEQ ID NO: 44 | SEQ ID NO: 39 |
| SEQ ID NO: 179 | SEQ ID NO: 44 | SEQ ID NO: 39 |
| SEQ ID NO: 180 | SEQ ID NO: 44 | SEQ ID NO: 39 |
| SEQ ID NO: 181 | SEQ ID NO: 44 | SEQ ID NO: 39 |
| SEQ ID NO: 182 | SEQ ID NO: 44 | SEQ ID NO: 39 |
| SEQ ID NO: 183 | SEQ ID NO: 44 | SEQ ID NO: 39 |
| SEQ ID NO: 184 | SEQ ID NO: 44 | SEQ ID NO: 39 |
| SEQ ID NO: 185 | SEQ ID NO: 44 | SEQ ID NO: 39 |
| SEQ ID NO: 186 | SEQ ID NO: 44 | SEQ ID NO: 39 |
| SEQ ID NO: 187 | SEQ ID NO: 44 | SEQ ID NO: 39 |
| SEQ ID NO: 188 | SEQ ID NO: 44 | SEQ ID NO: 39 |
| SEQ ID NO: 189 | SEQ ID NO: 44 | SEQ ID NO: 39 |
| SEQ ID NO: 190 | SEQ ID NO: 44 | SEQ ID NO: 39 |
| SEQ ID NO: 191 | SEQ ID NO: 44 | SEQ ID NO: 39 |
| SEQ ID NO: 192 | SEQ ID NO: 44 | SEQ ID NO: 39 |
| SEQ ID NO: 193 | SEQ ID NO: 44 | SEQ ID NO: 39 |
| SEQ ID NO: 194 | SEQ ID NO: 44 | SEQ ID NO: 39 |
| SEQ ID NO: 195 | SEQ ID NO: 44 | SEQ ID NO: 39 |
| SEQ ID NO: 196 | SEQ ID NO: 44 | SEQ ID NO: 39 |
| SEQ ID NO: 197 | SEQ ID NO: 44 | SEQ ID NO: 39 |
| SEQ ID NO: 198 | SEQ ID NO: 44 | SEQ ID NO: 39 |
| SEQ ID NO: 199 | SEQ ID NO: 44 | SEQ ID NO: 39 |
| SEQ ID NO: 200 | SEQ ID NO: 44 | SEQ ID NO: 39 |
| SEQ ID NO: 201 | SEQ ID NO: 44 | SEQ ID NO: 39 |
| SEQ ID NO: 202 | SEQ ID NO: 44 | SEQ ID NO: 39 |
| SEQ ID NO: 203 | SEQ ID NO: 44 | SEQ ID NO: 39 |
| SEQ ID NO: 204 | SEQ ID NO: 44 | SEQ ID NO: 39 |
| SEQ ID NO: 205 | SEQ ID NO: 44 | SEQ ID NO: 39 |
| SEQ ID NO: 206 | SEQ ID NO: 44 | SEQ ID NO: 39 |
| SEQ ID NO: 207 | SEQ ID NO: 44 | SEQ ID NO: 39 |
| SEQ ID NO: 208 | SEQ ID NO: 44 | SEQ ID NO: 39 |
| SEQ ID NO: 209 | SEQ ID NO: 44 | SEQ ID NO: 39 |
| SEQ ID NO: 210 | SEQ ID NO: 44 | SEQ ID NO: 39 |
| SEQ ID NO: 211 | SEQ ID NO: 44 | SEQ ID NO: 39 |
| SEQ ID NO: 212 | SEQ ID NO: 44 | SEQ ID NO: 39 |
| SEQ ID NO: 213 | SEQ ID NO: 44 | SEQ ID NO: 39 |
| SEQ ID NO: 214 | SEQ ID NO: 44 | SEQ ID NO: 39 |
| SEQ ID NO: 215 | SEQ ID NO: 44 | SEQ ID NO: 39 |
| SEQ ID NO: 216 | SEQ ID NO: 44 | SEQ ID NO: 39 |
| SEQ ID NO: 217 | SEQ ID NO: 44 | SEQ ID NO: 39 |
| SEQ ID NO: 218 | SEQ ID NO: 44 | SEQ ID NO: 39 |
| SEQ ID NO: 219 | SEQ ID NO: 44 | SEQ ID NO: 39 |
| SEQ ID NO: 220 | SEQ ID NO: 44 | SEQ ID NO: 39 |
| SEQ ID NO: 221 | SEQ ID NO: 44 | SEQ ID NO: 39 |
| SEQ ID NO: 329 | SEQ ID NO: 44 | SEQ ID NO: 39 |
| SEQ ID NO: 332 | SEQ ID NO: 44 | SEQ ID NO: 39 |
| SEQ ID NO: 335 | SEQ ID NO: 44 | SEQ ID NO: 39 |
| SEQ ID NO: 119 | SEQ ID NO: 44 | SEQ ID NO: 41 |
| SEQ ID NO: 120 | SEQ ID NO: 44 | SEQ ID NO: 41 |
| SEQ ID NO: 121 | SEQ ID NO: 44 | SEQ ID NO: 41 |
| SEQ ID NO: 122 | SEQ ID NO: 44 | SEQ ID NO: 41 |
| SEQ ID NO: 123 | SEQ ID NO: 44 | SEQ ID NO: 41 |
| SEQ ID NO: 124 | SEQ ID NO: 44 | SEQ ID NO: 41 |
| SEQ ID NO: 125 | SEQ ID NO: 44 | SEQ ID NO: 41 |
| SEQ ID NO: 126 | SEQ ID NO: 44 | SEQ ID NO: 41 |
| SEQ ID NO: 127 | SEQ ID NO: 44 | SEQ ID NO: 41 |
| SEQ ID NO: 128 | SEQ ID NO: 44 | SEQ ID NO: 41 |
| SEQ ID NO: 129 | SEQ ID NO: 44 | SEQ ID NO: 41 |
| SEQ ID NO: 130 | SEQ ID NO: 44 | SEQ ID NO: 41 |
| SEQ ID NO: 131 | SEQ ID NO: 44 | SEQ ID NO: 41 |
| SEQ ID NO: 132 | SEQ ID NO: 44 | SEQ ID NO: 41 |
| SEQ ID NO: 133 | SEQ ID NO: 44 | SEQ ID NO: 41 |
| SEQ ID NO: 134 | SEQ ID NO: 44 | SEQ ID NO: 41 |
| SEQ ID NO: 135 | SEQ ID NO: 44 | SEQ ID NO: 41 |
| SEQ ID NO: 136 | SEQ ID NO: 44 | SEQ ID NO: 41 |
| SEQ ID NO: 137 | SEQ ID NO: 44 | SEQ ID NO: 41 |
| SEQ ID NO: 138 | SEQ ID NO: 44 | SEQ ID NO: 41 |
| SEQ ID NO: 139 | SEQ ID NO: 44 | SEQ ID NO: 41 |
| SEQ ID NO: 140 | SEQ ID NO: 44 | SEQ ID NO: 41 |
| SEQ ID NO: 141 | SEQ ID NO: 44 | SEQ ID NO: 41 |
| SEQ ID NO: 142 | SEQ ID NO: 44 | SEQ ID NO: 41 |
| SEQ ID NO: 143 | SEQ ID NO: 44 | SEQ ID NO: 41 |
| SEQ ID NO: 144 | SEQ ID NO: 44 | SEQ ID NO: 41 |
| SEQ ID NO: 145 | SEQ ID NO: 44 | SEQ ID NO: 41 |
| SEQ ID NO: 146 | SEQ ID NO: 44 | SEQ ID NO: 41 |
| SEQ ID NO: 147 | SEQ ID NO: 44 | SEQ ID NO: 41 |
| SEQ ID NO: 148 | SEQ ID NO: 44 | SEQ ID NO: 41 |
| SEQ ID NO: 149 | SEQ ID NO: 44 | SEQ ID NO: 41 |
| SEQ ID NO: 150 | SEQ ID NO: 44 | SEQ ID NO: 41 |
| SEQ ID NO: 151 | SEQ ID NO: 44 | SEQ ID NO: 41 |
| SEQ ID NO: 152 | SEQ ID NO: 44 | SEQ ID NO: 41 |
| SEQ ID NO: 153 | SEQ ID NO: 44 | SEQ ID NO: 41 |
| SEQ ID NO: 154 | SEQ ID NO: 44 | SEQ ID NO: 41 |
| SEQ ID NO: 155 | SEQ ID NO: 44 | SEQ ID NO: 41 |
| SEQ ID NO: 156 | SEQ ID NO: 44 | SEQ ID NO: 41 |
| SEQ ID NO: 157 | SEQ ID NO: 44 | SEQ ID NO: 41 |
| SEQ ID NO: 158 | SEQ ID NO: 44 | SEQ ID NO: 41 |
| SEQ ID NO: 159 | SEQ ID NO: 44 | SEQ ID NO: 41 |
| SEQ ID NO: 160 | SEQ ID NO: 44 | SEQ ID NO: 41 |
| SEQ ID NO: 161 | SEQ ID NO: 44 | SEQ ID NO: 41 |
| SEQ ID NO: 162 | SEQ ID NO: 44 | SEQ ID NO: 41 |
| SEQ ID NO: 163 | SEQ ID NO: 44 | SEQ ID NO: 41 |
| SEQ ID NO: 164 | SEQ ID NO: 44 | SEQ ID NO: 41 |
| SEQ ID NO: 165 | SEQ ID NO: 44 | SEQ ID NO: 41 |
| SEQ ID NO: 166 | SEQ ID NO: 44 | SEQ ID NO: 41 |
| SEQ ID NO: 167 | SEQ ID NO: 44 | SEQ ID NO: 41 |
| SEQ ID NO: 168 | SEQ ID NO: 44 | SEQ ID NO: 41 |
| SEQ ID NO: 169 | SEQ ID NO: 44 | SEQ ID NO: 41 |
| SEQ ID NO: 170 | SEQ ID NO: 44 | SEQ ID NO: 41 |
| SEQ ID NO: 171 | SEQ ID NO: 44 | SEQ ID NO: 41 |
| SEQ ID NO: 172 | SEQ ID NO: 44 | SEQ ID NO: 41 |
| SEQ ID NO: 173 | SEQ ID NO: 44 | SEQ ID NO: 41 |
| SEQ ID NO: 174 | SEQ ID NO: 44 | SEQ ID NO: 41 |
| SEQ ID NO: 175 | SEQ ID NO: 44 | SEQ ID NO: 41 |
| SEQ ID NO: 176 | SEQ ID NO: 44 | SEQ ID NO: 41 |
| SEQ ID NO: 177 | SEQ ID NO: 44 | SEQ ID NO: 41 |
| SEQ ID NO: 178 | SEQ ID NO: 44 | SEQ ID NO: 41 |
| SEQ ID NO: 179 | SEQ ID NO: 44 | SEQ ID NO: 41 |
| SEQ ID NO: 180 | SEQ ID NO: 44 | SEQ ID NO: 41 |
| SEQ ID NO: 181 | SEQ ID NO: 44 | SEQ ID NO: 41 |
| SEQ ID NO: 182 | SEQ ID NO: 44 | SEQ ID NO: 41 |
| SEQ ID NO: 183 | SEQ ID NO: 44 | SEQ ID NO: 41 |
| SEQ ID NO: 184 | SEQ ID NO: 44 | SEQ ID NO: 41 |
| SEQ ID NO: 185 | SEQ ID NO: 44 | SEQ ID NO: 41 |
| SEQ ID NO: 186 | SEQ ID NO: 44 | SEQ ID NO: 41 |
| SEQ ID NO: 187 | SEQ ID NO: 44 | SEQ ID NO: 41 |
| SEQ ID NO: 188 | SEQ ID NO: 44 | SEQ ID NO: 41 |
| SEQ ID NO: 189 | SEQ ID NO: 44 | SEQ ID NO: 41 |
| SEQ ID NO: 190 | SEQ ID NO: 44 | SEQ ID NO: 41 |
| SEQ ID NO: 191 | SEQ ID NO: 44 | SEQ ID NO: 41 |
| SEQ ID NO: 192 | SEQ ID NO: 44 | SEQ ID NO: 41 |
| SEQ ID NO: 193 | SEQ ID NO: 44 | SEQ ID NO: 41 |
| SEQ ID NO: 194 | SEQ ID NO: 44 | SEQ ID NO: 41 |
| SEQ ID NO: 195 | SEQ ID NO: 44 | SEQ ID NO: 41 |
| SEQ ID NO: 196 | SEQ ID NO: 44 | SEQ ID NO: 41 |
| SEQ ID NO: 197 | SEQ ID NO: 44 | SEQ ID NO: 41 |
| SEQ ID NO: 198 | SEQ ID NO: 44 | SEQ ID NO: 41 |
| SEQ ID NO: 199 | SEQ ID NO: 44 | SEQ ID NO: 41 |
| SEQ ID NO: 200 | SEQ ID NO: 44 | SEQ ID NO: 41 |
| SEQ ID NO: 201 | SEQ ID NO: 44 | SEQ ID NO: 41 |
| SEQ ID NO: 202 | SEQ ID NO: 44 | SEQ ID NO: 41 |
| SEQ ID NO: 203 | SEQ ID NO: 44 | SEQ ID NO: 41 |
| SEQ ID NO: 204 | SEQ ID NO: 44 | SEQ ID NO: 41 |
| SEQ ID NO: 205 | SEQ ID NO: 44 | SEQ ID NO: 41 |
| SEQ ID NO: 206 | SEQ ID NO: 44 | SEQ ID NO: 41 |
| SEQ ID NO: 207 | SEQ ID NO: 44 | SEQ ID NO: 41 |
| SEQ ID NO: 208 | SEQ ID NO: 44 | SEQ ID NO: 41 |
| SEQ ID NO: 209 | SEQ ID NO: 44 | SEQ ID NO: 41 |
| SEQ ID NO: 210 | SEQ ID NO: 44 | SEQ ID NO: 41 |
| SEQ ID NO: 211 | SEQ ID NO: 44 | SEQ ID NO: 41 |
| SEQ ID NO: 212 | SEQ ID NO: 44 | SEQ ID NO: 41 |
| SEQ ID NO: 213 | SEQ ID NO: 44 | SEQ ID NO: 41 |
| SEQ ID NO: 214 | SEQ ID NO: 44 | SEQ ID NO: 41 |
| SEQ ID NO: 215 | SEQ ID NO: 44 | SEQ ID NO: 41 |
| SEQ ID NO: 216 | SEQ ID NO: 44 | SEQ ID NO: 41 |
| SEQ ID NO: 217 | SEQ ID NO: 44 | SEQ ID NO: 41 |
| SEQ ID NO: 218 | SEQ ID NO: 44 | SEQ ID NO: 41 |
| SEQ ID NO: 219 | SEQ ID NO: 44 | SEQ ID NO: 41 |
| SEQ ID NO: 220 | SEQ ID NO: 44 | SEQ ID NO: 41 |
| SEQ ID NO: 221 | SEQ ID NO: 44 | SEQ ID NO: 41 |
| SEQ ID NO: 329 | SEQ ID NO: 44 | SEQ ID NO: 41 |
| SEQ ID NO: 332 | SEQ ID NO: 44 | SEQ ID NO: 41 |
| SEQ ID NO: 335 | SEQ ID NO: 44 | SEQ ID NO: 41 |
| SEQ ID NO: 119 | SEQ ID NO: 44 | SEQ ID NO: 43 |
| SEQ ID NO: 120 | SEQ ID NO: 44 | SEQ ID NO: 43 |
| SEQ ID NO: 121 | SEQ ID NO: 44 | SEQ ID NO: 43 |
| SEQ ID NO: 122 | SEQ ID NO: 44 | SEQ ID NO: 43 |
| SEQ ID NO: 123 | SEQ ID NO: 44 | SEQ ID NO: 43 |
| SEQ ID NO: 124 | SEQ ID NO: 44 | SEQ ID NO: 43 |
| SEQ ID NO: 125 | SEQ ID NO: 44 | SEQ ID NO: 43 |
| SEQ ID NO: 126 | SEQ ID NO: 44 | SEQ ID NO: 43 |
| SEQ ID NO: 127 | SEQ ID NO: 44 | SEQ ID NO: 43 |
| SEQ ID NO: 128 | SEQ ID NO: 44 | SEQ ID NO: 43 |
| SEQ ID NO: 129 | SEQ ID NO: 44 | SEQ ID NO: 43 |
| SEQ ID NO: 130 | SEQ ID NO: 44 | SEQ ID NO: 43 |
| SEQ ID NO: 131 | SEQ ID NO: 44 | SEQ ID NO: 43 |
| SEQ ID NO: 132 | SEQ ID NO: 44 | SEQ ID NO: 43 |
| SEQ ID NO: 133 | SEQ ID NO: 44 | SEQ ID NO: 43 |
| SEQ ID NO: 134 | SEQ ID NO: 44 | SEQ ID NO: 43 |
| SEQ ID NO: 135 | SEQ ID NO: 44 | SEQ ID NO: 43 |
| SEQ ID NO: 136 | SEQ ID NO: 44 | SEQ ID NO: 43 |
| SEQ ID NO: 137 | SEQ ID NO: 44 | SEQ ID NO: 43 |
| SEQ ID NO: 138 | SEQ ID NO: 44 | SEQ ID NO: 43 |
| SEQ ID NO: 139 | SEQ ID NO: 44 | SEQ ID NO: 43 |
| SEQ ID NO: 140 | SEQ ID NO: 44 | SEQ ID NO: 43 |
| SEQ ID NO: 141 | SEQ ID NO: 44 | SEQ ID NO: 43 |
| SEQ ID NO: 142 | SEQ ID NO: 44 | SEQ ID NO: 43 |
| SEQ ID NO: 143 | SEQ ID NO: 44 | SEQ ID NO: 43 |
| SEQ ID NO: 144 | SEQ ID NO: 44 | SEQ ID NO: 43 |
| SEQ ID NO: 145 | SEQ ID NO: 44 | SEQ ID NO: 43 |
| SEQ ID NO: 146 | SEQ ID NO: 44 | SEQ ID NO: 43 |
| SEQ ID NO: 147 | SEQ ID NO: 44 | SEQ ID NO: 43 |
| SEQ ID NO: 148 | SEQ ID NO: 44 | SEQ ID NO: 43 |
| SEQ ID NO: 149 | SEQ ID NO: 44 | SEQ ID NO: 43 |
| SEQ ID NO: 150 | SEQ ID NO: 44 | SEQ ID NO: 43 |
| SEQ ID NO: 151 | SEQ ID NO: 44 | SEQ ID NO: 43 |
| SEQ ID NO: 152 | SEQ ID NO: 44 | SEQ ID NO: 43 |
| SEQ ID NO: 153 | SEQ ID NO: 44 | SEQ ID NO: 43 |
| SEQ ID NO: 154 | SEQ ID NO: 44 | SEQ ID NO: 43 |
| SEQ ID NO: 155 | SEQ ID NO: 44 | SEQ ID NO: 43 |
| SEQ ID NO: 156 | SEQ ID NO: 44 | SEQ ID NO: 43 |
| SEQ ID NO: 157 | SEQ ID NO: 44 | SEQ ID NO: 43 |
| SEQ ID NO: 158 | SEQ ID NO: 44 | SEQ ID NO: 43 |
| SEQ ID NO: 159 | SEQ ID NO: 44 | SEQ ID NO: 43 |
| SEQ ID NO: 160 | SEQ ID NO: 44 | SEQ ID NO: 43 |
| SEQ ID NO: 161 | SEQ ID NO: 44 | SEQ ID NO: 43 |
| SEQ ID NO: 162 | SEQ ID NO: 44 | SEQ ID NO: 43 |
| SEQ ID NO: 163 | SEQ ID NO: 44 | SEQ ID NO: 43 |
| SEQ ID NO: 164 | SEQ ID NO: 44 | SEQ ID NO: 43 |
| SEQ ID NO: 165 | SEQ ID NO: 44 | SEQ ID NO: 43 |
| SEQ ID NO: 166 | SEQ ID NO: 44 | SEQ ID NO: 43 |
| SEQ ID NO: 167 | SEQ ID NO: 44 | SEQ ID NO: 43 |
| SEQ ID NO: 168 | SEQ ID NO: 44 | SEQ ID NO: 43 |
| SEQ ID NO: 169 | SEQ ID NO: 44 | SEQ ID NO: 43 |
| SEQ ID NO: 170 | SEQ ID NO: 44 | SEQ ID NO: 43 |
| SEQ ID NO: 171 | SEQ ID NO: 44 | SEQ ID NO: 43 |
| SEQ ID NO: 172 | SEQ ID NO: 44 | SEQ ID NO: 43 |
| SEQ ID NO: 173 | SEQ ID NO: 44 | SEQ ID NO: 43 |
| SEQ ID NO: 174 | SEQ ID NO: 44 | SEQ ID NO: 43 |
| SEQ ID NO: 175 | SEQ ID NO: 44 | SEQ ID NO: 43 |
| SEQ ID NO: 176 | SEQ ID NO: 44 | SEQ ID NO: 43 |
| SEQ ID NO: 177 | SEQ ID NO: 44 | SEQ ID NO: 43 |
| SEQ ID NO: 178 | SEQ ID NO: 44 | SEQ ID NO: 43 |
| SEQ ID NO: 179 | SEQ ID NO: 44 | SEQ ID NO: 43 |
| SEQ ID NO: 180 | SEQ ID NO: 44 | SEQ ID NO: 43 |
| SEQ ID NO: 181 | SEQ ID NO: 44 | SEQ ID NO: 43 |
| SEQ ID NO: 182 | SEQ ID NO: 44 | SEQ ID NO: 43 |
| SEQ ID NO: 183 | SEQ ID NO: 44 | SEQ ID NO: 43 |
| SEQ ID NO: 184 | SEQ ID NO: 44 | SEQ ID NO: 43 |
| SEQ ID NO: 185 | SEQ ID NO: 44 | SEQ ID NO: 43 |
| SEQ ID NO: 186 | SEQ ID NO: 44 | SEQ ID NO: 43 |
| SEQ ID NO: 187 | SEQ ID NO: 44 | SEQ ID NO: 43 |
| SEQ ID NO: 188 | SEQ ID NO: 44 | SEQ ID NO: 43 |
| SEQ ID NO: 189 | SEQ ID NO: 44 | SEQ ID NO: 43 |
| SEQ ID NO: 190 | SEQ ID NO: 44 | SEQ ID NO: 43 |
| SEQ ID NO: 191 | SEQ ID NO: 44 | SEQ ID NO: 43 |
| SEQ ID NO: 192 | SEQ ID NO: 44 | SEQ ID NO: 43 |
| SEQ ID NO: 193 | SEQ ID NO: 44 | SEQ ID NO: 43 |
| SEQ ID NO: 194 | SEQ ID NO: 44 | SEQ ID NO: 43 |
| SEQ ID NO: 195 | SEQ ID NO: 44 | SEQ ID NO: 43 |
| SEQ ID NO: 196 | SEQ ID NO: 44 | SEQ ID NO: 43 |
| SEQ ID NO: 197 | SEQ ID NO: 44 | SEQ ID NO: 43 |
| SEQ ID NO: 198 | SEQ ID NO: 44 | SEQ ID NO: 43 |
| SEQ ID NO: 199 | SEQ ID NO: 44 | SEQ ID NO: 43 |
| SEQ ID NO: 200 | SEQ ID NO: 44 | SEQ ID NO: 43 |
| SEQ ID NO: 201 | SEQ ID NO: 44 | SEQ ID NO: 43 |
| SEQ ID NO: 202 | SEQ ID NO: 44 | SEQ ID NO: 43 |
| SEQ ID NO: 203 | SEQ ID NO: 44 | SEQ ID NO: 43 |
| SEQ ID NO: 204 | SEQ ID NO: 44 | SEQ ID NO: 43 |
| SEQ ID NO: 205 | SEQ ID NO: 44 | SEQ ID NO: 43 |
| SEQ ID NO: 206 | SEQ ID NO: 44 | SEQ ID NO: 43 |
| SEQ ID NO: 207 | SEQ ID NO: 44 | SEQ ID NO: 43 |
| SEQ ID NO: 208 | SEQ ID NO: 44 | SEQ ID NO: 43 |
| SEQ ID NO: 209 | SEQ ID NO: 44 | SEQ ID NO: 43 |
| SEQ ID NO: 210 | SEQ ID NO: 44 | SEQ ID NO: 43 |
| SEQ ID NO: 211 | SEQ ID NO: 44 | SEQ ID NO: 43 |
| SEQ ID NO: 212 | SEQ ID NO: 44 | SEQ ID NO: 43 |
| SEQ ID NO: 213 | SEQ ID NO: 44 | SEQ ID NO: 43 |
| SEQ ID NO: 214 | SEQ ID NO: 44 | SEQ ID NO: 43 |
| SEQ ID NO: 215 | SEQ ID NO: 44 | SEQ ID NO: 43 |
| SEQ ID NO: 216 | SEQ ID NO: 44 | SEQ ID NO: 43 |
| SEQ ID NO: 217 | SEQ ID NO: 44 | SEQ ID NO: 43 |
| SEQ ID NO: 218 | SEQ ID NO: 44 | SEQ ID NO: 43 |
| SEQ ID NO: 219 | SEQ ID NO: 44 | SEQ ID NO: 43 |
| SEQ ID NO: 220 | SEQ ID NO: 44 | SEQ ID NO: 43 |
| SEQ ID NO: 221 | SEQ ID NO: 44 | SEQ ID NO: 43 |
| SEQ ID NO: 329 | SEQ ID NO: 44 | SEQ ID NO: 43 |
| SEQ ID NO: 332 | SEQ ID NO: 44 | SEQ ID NO: 43 |
| SEQ ID NO: 335 | SEQ ID NO: 44 | SEQ ID NO: 43 |

TABLE 4
Example configurations of fusion proteins
Components (from an N-terminus to C-terminus direction)

| Mutant Soluble | | |
|---|---|---|
| ActRIIB-ECD polypeptide | Linker(s) | Heterologous Protein |
| SEQ ID NO: 119 | SEQ ID NO: 118 | SEQ ID NO: 39 |
| SEQ ID NO: 120 | SEQ ID NO: 118 | SEQ ID NO: 39 |
| SEQ ID NO: 121 | SEQ ID NO: 118 | SEQ ID NO: 39 |
| SEQ ID NO: 122 | SEQ ID NO: 118 | SEQ ID NO: 39 |
| SEQ ID NO: 123 | SEQ ID NO: 118 | SEQ ID NO: 39 |
| SEQ ID NO: 124 | SEQ ID NO: 118 | SEQ ID NO: 39 |
| SEQ ID NO: 125 | SEQ ID NO: 118 | SEQ ID NO: 39 |
| SEQ ID NO: 126 | SEQ ID NO: 118 | SEQ ID NO: 39 |
| SEQ ID NO: 127 | SEQ ID NO: 118 | SEQ ID NO: 39 |
| SEQ ID NO: 128 | SEQ ID NO: 118 | SEQ ID NO: 39 |
| SEQ ID NO: 129 | SEQ ID NO: 118 | SEQ ID NO: 39 |
| SEQ ID NO: 130 | SEQ ID NO: 118 | SEQ ID NO: 39 |
| SEQ ID NO: 131 | SEQ ID NO: 118 | SEQ ID NO: 39 |
| SEQ ID NO: 132 | SEQ ID NO: 118 | SEQ ID NO: 39 |
| SEQ ID NO: 133 | SEQ ID NO: 118 | SEQ ID NO: 39 |
| SEQ ID NO: 134 | SEQ ID NO: 118 | SEQ ID NO: 39 |
| SEQ ID NO: 135 | SEQ ID NO: 118 | SEQ ID NO: 39 |
| SEQ ID NO: 136 | SEQ ID NO: 118 | SEQ ID NO: 39 |
| SEQ ID NO: 137 | SEQ ID NO: 118 | SEQ ID NO: 39 |
| SEQ ID NO: 138 | SEQ ID NO: 118 | SEQ ID NO: 39 |
| SEQ ID NO: 139 | SEQ ID NO: 118 | SEQ ID NO: 39 |
| SEQ ID NO: 140 | SEQ ID NO: 118 | SEQ ID NO: 39 |
| SEQ ID NO: 141 | SEQ ID NO: 118 | SEQ ID NO: 39 |
| SEQ ID NO: 142 | SEQ ID NO: 118 | SEQ ID NO: 39 |
| SEQ ID NO: 143 | SEQ ID NO: 118 | SEQ ID NO: 39 |
| SEQ ID NO: 144 | SEQ ID NO: 118 | SEQ ID NO: 39 |
| SEQ ID NO: 145 | SEQ ID NO: 118 | SEQ ID NO: 39 |
| SEQ ID NO: 146 | SEQ ID NO: 118 | SEQ ID NO: 39 |
| SEQ ID NO: 147 | SEQ ID NO: 118 | SEQ ID NO: 39 |
| SEQ ID NO: 148 | SEQ ID NO: 118 | SEQ ID NO: 39 |
| SEQ ID NO: 149 | SEQ ID NO: 118 | SEQ ID NO: 39 |
| SEQ ID NO: 150 | SEQ ID NO: 118 | SEQ ID NO: 39 |
| SEQ ID NO: 151 | SEQ ID NO: 118 | SEQ ID NO: 39 |
| SEQ ID NO: 152 | SEQ ID NO: 118 | SEQ ID NO: 39 |
| SEQ ID NO: 153 | SEQ ID NO: 118 | SEQ ID NO: 39 |
| SEQ ID NO: 154 | SEQ ID NO: 118 | SEQ ID NO: 39 |
| SEQ ID NO: 155 | SEQ ID NO: 118 | SEQ ID NO: 39 |
| SEQ ID NO: 156 | SEQ ID NO: 118 | SEQ ID NO: 39 |
| SEQ ID NO: 157 | SEQ ID NO: 118 | SEQ ID NO: 39 |
| SEQ ID NO: 158 | SEQ ID NO: 118 | SEQ ID NO: 39 |
| SEQ ID NO: 159 | SEQ ID NO: 118 | SEQ ID NO: 39 |
| SEQ ID NO: 160 | SEQ ID NO: 118 | SEQ ID NO: 39 |
| SEQ ID NO: 161 | SEQ ID NO: 118 | SEQ ID NO: 39 |
| SEQ ID NO: 162 | SEQ ID NO: 118 | SEQ ID NO: 39 |
| SEQ ID NO: 163 | SEQ ID NO: 118 | SEQ ID NO: 39 |
| SEQ ID NO: 164 | SEQ ID NO: 118 | SEQ ID NO: 39 |
| SEQ ID NO: 165 | SEQ ID NO: 118 | SEQ ID NO: 39 |
| SEQ ID NO: 166 | SEQ ID NO: 118 | SEQ ID NO: 39 |
| SEQ ID NO: 167 | SEQ ID NO: 118 | SEQ ID NO: 39 |
| SEQ ID NO: 168 | SEQ ID NO: 118 | SEQ ID NO: 39 |
| SEQ ID NO: 169 | SEQ ID NO: 118 | SEQ ID NO: 39 |
| SEQ ID NO: 170 | SEQ ID NO: 118 | SEQ ID NO: 39 |
| SEQ ID NO: 171 | SEQ ID NO: 118 | SEQ ID NO: 39 |
| SEQ ID NO: 172 | SEQ ID NO: 118 | SEQ ID NO: 39 |
| SEQ ID NO: 173 | SEQ ID NO: 118 | SEQ ID NO: 39 |
| SEQ ID NO: 174 | SEQ ID NO: 118 | SEQ ID NO: 39 |
| SEQ ID NO: 175 | SEQ ID NO: 118 | SEQ ID NO: 39 |
| SEQ ID NO: 176 | SEQ ID NO: 118 | SEQ ID NO: 39 |
| SEQ ID NO: 177 | SEQ ID NO: 118 | SEQ ID NO: 39 |
| SEQ ID NO: 178 | SEQ ID NO: 118 | SEQ ID NO: 39 |
| SEQ ID NO: 179 | SEQ ID NO: 118 | SEQ ID NO: 39 |
| SEQ ID NO: 180 | SEQ ID NO: 118 | SEQ ID NO: 39 |
| SEQ ID NO: 181 | SEQ ID NO: 118 | SEQ ID NO: 39 |
| SEQ ID NO: 182 | SEQ ID NO: 118 | SEQ ID NO: 39 |
| SEQ ID NO: 183 | SEQ ID NO: 118 | SEQ ID NO: 39 |
| SEQ ID NO: 184 | SEQ ID NO: 118 | SEQ ID NO: 39 |
| SEQ ID NO: 185 | SEQ ID NO: 118 | SEQ ID NO: 39 |
| SEQ ID NO: 186 | SEQ ID NO: 118 | SEQ ID NO: 39 |
| SEQ ID NO: 187 | SEQ ID NO: 118 | SEQ ID NO: 39 |
| SEQ ID NO: 188 | SEQ ID NO: 118 | SEQ ID NO: 39 |
| SEQ ID NO: 189 | SEQ ID NO: 118 | SEQ ID NO: 39 |
| SEQ ID NO: 190 | SEQ ID NO: 118 | SEQ ID NO: 39 |
| SEQ ID NO: 191 | SEQ ID NO: 118 | SEQ ID NO: 39 |
| SEQ ID NO: 192 | SEQ ID NO: 118 | SEQ ID NO: 39 |
| SEQ ID NO: 193 | SEQ ID NO: 118 | SEQ ID NO: 39 |
| SEQ ID NO: 194 | SEQ ID NO: 118 | SEQ ID NO: 39 |
| SEQ ID NO: 195 | SEQ ID NO: 118 | SEQ ID NO: 39 |
| SEQ ID NO: 196 | SEQ ID NO: 118 | SEQ ID NO: 39 |
| SEQ ID NO: 197 | SEQ ID NO: 118 | SEQ ID NO: 39 |
| SEQ ID NO: 198 | SEQ ID NO: 118 | SEQ ID NO: 39 |
| SEQ ID NO: 199 | SEQ ID NO: 118 | SEQ ID NO: 39 |
| SEQ ID NO: 200 | SEQ ID NO: 118 | SEQ ID NO: 39 |
| SEQ ID NO: 201 | SEQ ID NO: 118 | SEQ ID NO: 39 |
| SEQ ID NO: 202 | SEQ ID NO: 118 | SEQ ID NO: 39 |
| SEQ ID NO: 203 | SEQ ID NO: 118 | SEQ ID NO: 39 |
| SEQ ID NO: 204 | SEQ ID NO: 118 | SEQ ID NO: 39 |
| SEQ ID NO: 205 | SEQ ID NO: 118 | SEQ ID NO: 39 |
| SEQ ID NO: 206 | SEQ ID NO: 118 | SEQ ID NO: 39 |
| SEQ ID NO: 207 | SEQ ID NO: 118 | SEQ ID NO: 39 |
| SEQ ID NO: 208 | SEQ ID NO: 118 | SEQ ID NO: 39 |
| SEQ ID NO: 209 | SEQ ID NO: 118 | SEQ ID NO: 39 |
| SEQ ID NO: 210 | SEQ ID NO: 118 | SEQ ID NO: 39 |
| SEQ ID NO: 211 | SEQ ID NO: 118 | SEQ ID NO: 39 |
| SEQ ID NO: 212 | SEQ ID NO: 118 | SEQ ID NO: 39 |
| SEQ ID NO: 213 | SEQ ID NO: 118 | SEQ ID NO: 39 |
| SEQ ID NO: 214 | SEQ ID NO: 118 | SEQ ID NO: 39 |
| SEQ ID NO: 215 | SEQ ID NO: 118 | SEQ ID NO: 39 |
| SEQ ID NO: 216 | SEQ ID NO: 118 | SEQ ID NO: 39 |
| SEQ ID NO: 217 | SEQ ID NO: 118 | SEQ ID NO: 39 |
| SEQ ID NO: 218 | SEQ ID NO: 118 | SEQ ID NO: 39 |
| SEQ ID NO: 219 | SEQ ID NO: 118 | SEQ ID NO: 39 |
| SEQ ID NO: 220 | SEQ ID NO: 118 | SEQ ID NO: 39 |
| SEQ ID NO: 221 | SEQ ID NO: 118 | SEQ ID NO: 39 |
| SEQ ID NO: 329 | SEQ ID NO: 118 | SEQ ID NO: 39 |
| SEQ ID NO: 332 | SEQ ID NO: 118 | SEQ ID NO: 39 |
| SEQ ID NO: 335 | SEQ ID NO: 118 | SEQ ID NO: 39 |
| SEQ ID NO: 119 | SEQ ID NO: 118 | SEQ ID NO: 41 |
| SEQ ID NO: 120 | SEQ ID NO: 118 | SEQ ID NO: 41 |
| SEQ ID NO: 121 | SEQ ID NO: 118 | SEQ ID NO: 41 |
| SEQ ID NO: 122 | SEQ ID NO: 118 | SEQ ID NO: 41 |
| SEQ ID NO: 123 | SEQ ID NO: 118 | SEQ ID NO: 41 |
| SEQ ID NO: 124 | SEQ ID NO: 118 | SEQ ID NO: 41 |
| SEQ ID NO: 125 | SEQ ID NO: 118 | SEQ ID NO: 41 |
| SEQ ID NO: 126 | SEQ ID NO: 118 | SEQ ID NO: 41 |
| SEQ ID NO: 127 | SEQ ID NO: 118 | SEQ ID NO: 41 |
| SEQ ID NO: 128 | SEQ ID NO: 118 | SEQ ID NO: 41 |
| SEQ ID NO: 129 | SEQ ID NO: 118 | SEQ ID NO: 41 |
| SEQ ID NO: 130 | SEQ ID NO: 118 | SEQ ID NO: 41 |
| SEQ ID NO: 131 | SEQ ID NO: 118 | SEQ ID NO: 41 |
| SEQ ID NO: 132 | SEQ ID NO: 118 | SEQ ID NO: 41 |
| SEQ ID NO: 133 | SEQ ID NO: 118 | SEQ ID NO: 41 |
| SEQ ID NO: 134 | SEQ ID NO: 118 | SEQ ID NO: 41 |
| SEQ ID NO: 135 | SEQ ID NO: 118 | SEQ ID NO: 41 |
| SEQ ID NO: 136 | SEQ ID NO: 118 | SEQ ID NO: 41 |
| SEQ ID NO: 137 | SEQ ID NO: 118 | SEQ ID NO: 41 |
| SEQ ID NO: 138 | SEQ ID NO: 118 | SEQ ID NO: 41 |
| SEQ ID NO: 139 | SEQ ID NO: 118 | SEQ ID NO: 41 |
| SEQ ID NO: 140 | SEQ ID NO: 118 | SEQ ID NO: 41 |
| SEQ ID NO: 141 | SEQ ID NO: 118 | SEQ ID NO: 41 |
| SEQ ID NO: 142 | SEQ ID NO: 118 | SEQ ID NO: 41 |
| SEQ ID NO: 143 | SEQ ID NO: 118 | SEQ ID NO: 41 |
| SEQ ID NO: 144 | SEQ ID NO: 118 | SEQ ID NO: 41 |
| SEQ ID NO: 145 | SEQ ID NO: 118 | SEQ ID NO: 41 |
| SEQ ID NO: 146 | SEQ ID NO: 118 | SEQ ID NO: 41 |
| SEQ ID NO: 147 | SEQ ID NO: 118 | SEQ ID NO: 41 |
| SEQ ID NO: 148 | SEQ ID NO: 118 | SEQ ID NO: 41 |
| SEQ ID NO: 149 | SEQ ID NO: 118 | SEQ ID NO: 41 |
| SEQ ID NO: 150 | SEQ ID NO: 118 | SEQ ID NO: 41 |
| SEQ ID NO: 151 | SEQ ID NO: 118 | SEQ ID NO: 41 |
| SEQ ID NO: 152 | SEQ ID NO: 118 | SEQ ID NO: 41 |
| SEQ ID NO: 153 | SEQ ID NO: 118 | SEQ ID NO: 41 |
| SEQ ID NO: 154 | SEQ ID NO: 118 | SEQ ID NO: 41 |
| SEQ ID NO: 155 | SEQ ID NO: 118 | SEQ ID NO: 41 |
| SEQ ID NO: 156 | SEQ ID NO: 118 | SEQ ID NO: 41 |
| SEQ ID NO: 157 | SEQ ID NO: 118 | SEQ ID NO: 41 |
| SEQ ID NO: 158 | SEQ ID NO: 118 | SEQ ID NO: 41 |
| SEQ ID NO: 159 | SEQ ID NO: 118 | SEQ ID NO: 41 |
| SEQ ID NO: 160 | SEQ ID NO: 118 | SEQ ID NO: 41 |
| SEQ ID NO: 161 | SEQ ID NO: 118 | SEQ ID NO: 41 |
| SEQ ID NO: 162 | SEQ ID NO: 118 | SEQ ID NO: 41 |
| SEQ ID NO: 163 | SEQ ID NO: 118 | SEQ ID NO: 41 |
| SEQ ID NO: 164 | SEQ ID NO: 118 | SEQ ID NO: 41 |
| SEQ ID NO: 165 | SEQ ID NO: 118 | SEQ ID NO: 41 |
| SEQ ID NO: 166 | SEQ ID NO: 118 | SEQ ID NO: 41 |
| SEQ ID NO: 167 | SEQ ID NO: 118 | SEQ ID NO: 41 |
| SEQ ID NO: 168 | SEQ ID NO: 118 | SEQ ID NO: 41 |
| SEQ ID NO: 169 | SEQ ID NO: 118 | SEQ ID NO: 41 |
| SEQ ID NO: 170 | SEQ ID NO: 118 | SEQ ID NO: 41 |
| SEQ ID NO: 171 | SEQ ID NO: 118 | SEQ ID NO: 41 |
| SEQ ID NO: 172 | SEQ ID NO: 118 | SEQ ID NO: 41 |
| SEQ ID NO: 173 | SEQ ID NO: 118 | SEQ ID NO: 41 |
| SEQ ID NO: 174 | SEQ ID NO: 118 | SEQ ID NO: 41 |
| SEQ ID NO: 175 | SEQ ID NO: 118 | SEQ ID NO: 41 |
| SEQ ID NO: 176 | SEQ ID NO: 118 | SEQ ID NO: 41 |
| SEQ ID NO: 177 | SEQ ID NO: 118 | SEQ ID NO: 41 |
| SEQ ID NO: 178 | SEQ ID NO: 118 | SEQ ID NO: 41 |
| SEQ ID NO: 179 | SEQ ID NO: 118 | SEQ ID NO: 41 |
| SEQ ID NO: 180 | SEQ ID NO: 118 | SEQ ID NO: 41 |
| SEQ ID NO: 181 | SEQ ID NO: 118 | SEQ ID NO: 41 |
| SEQ ID NO: 182 | SEQ ID NO: 118 | SEQ ID NO: 41 |
| SEQ ID NO: 183 | SEQ ID NO: 118 | SEQ ID NO: 41 |
| SEQ ID NO: 184 | SEQ ID NO: 118 | SEQ ID NO: 41 |
| SEQ ID NO: 185 | SEQ ID NO: 118 | SEQ ID NO: 41 |
| SEQ ID NO: 186 | SEQ ID NO: 118 | SEQ ID NO: 41 |
| SEQ ID NO: 187 | SEQ ID NO: 118 | SEQ ID NO: 41 |
| SEQ ID NO: 188 | SEQ ID NO: 118 | SEQ ID NO: 41 |
| SEQ ID NO: 189 | SEQ ID NO: 118 | SEQ ID NO: 41 |
| SEQ ID NO: 190 | SEQ ID NO: 118 | SEQ ID NO: 41 |
| SEQ ID NO: 191 | SEQ ID NO: 118 | SEQ ID NO: 41 |
| SEQ ID NO: 192 | SEQ ID NO: 118 | SEQ ID NO: 41 |
| SEQ ID NO: 193 | SEQ ID NO: 118 | SEQ ID NO: 41 |
| SEQ ID NO: 194 | SEQ ID NO: 118 | SEQ ID NO: 41 |
| SEQ ID NO: 195 | SEQ ID NO: 118 | SEQ ID NO: 41 |
| SEQ ID NO: 196 | SEQ ID NO: 118 | SEQ ID NO: 41 |
| SEQ ID NO: 197 | SEQ ID NO: 118 | SEQ ID NO: 41 |
| SEQ ID NO: 198 | SEQ ID NO: 118 | SEQ ID NO: 41 |
| SEQ ID NO: 199 | SEQ ID NO: 118 | SEQ ID NO: 41 |
| SEQ ID NO: 200 | SEQ ID NO: 118 | SEQ ID NO: 41 |
| SEQ ID NO: 201 | SEQ ID NO: 118 | SEQ ID NO: 41 |
| SEQ ID NO: 202 | SEQ ID NO: 118 | SEQ ID NO: 41 |
| SEQ ID NO: 203 | SEQ ID NO: 118 | SEQ ID NO: 41 |
| SEQ ID NO: 204 | SEQ ID NO: 118 | SEQ ID NO: 41 |
| SEQ ID NO: 205 | SEQ ID NO: 118 | SEQ ID NO: 41 |
| SEQ ID NO: 206 | SEQ ID NO: 118 | SEQ ID NO: 41 |
| SEQ ID NO: 207 | SEQ ID NO: 118 | SEQ ID NO: 41 |
| SEQ ID NO: 208 | SEQ ID NO: 118 | SEQ ID NO: 41 |
| SEQ ID NO: 209 | SEQ ID NO: 118 | SEQ ID NO: 41 |
| SEQ ID NO: 210 | SEQ ID NO: 118 | SEQ ID NO: 41 |
| SEQ ID NO: 211 | SEQ ID NO: 118 | SEQ ID NO: 41 |
| SEQ ID NO: 212 | SEQ ID NO: 118 | SEQ ID NO: 41 |
| SEQ ID NO: 213 | SEQ ID NO: 118 | SEQ ID NO: 41 |
| SEQ ID NO: 214 | SEQ ID NO: 118 | SEQ ID NO: 41 |
| SEQ ID NO: 215 | SEQ ID NO: 118 | SEQ ID NO: 41 |
| SEQ ID NO: 216 | SEQ ID NO: 118 | SEQ ID NO: 41 |
| SEQ ID NO: 217 | SEQ ID NO: 118 | SEQ ID NO: 41 |
| SEQ ID NO: 218 | SEQ ID NO: 118 | SEQ ID NO: 41 |
| SEQ ID NO: 219 | SEQ ID NO: 118 | SEQ ID NO: 41 |
| SEQ ID NO: 220 | SEQ ID NO: 118 | SEQ ID NO: 41 |
| SEQ ID NO: 221 | SEQ ID NO: 118 | SEQ ID NO: 41 |
| SEQ ID NO: 329 | SEQ ID NO: 118 | SEQ ID NO: 41 |
| SEQ ID NO: 332 | SEQ ID NO: 118 | SEQ ID NO: 41 |
| SEQ ID NO: 335 | SEQ ID NO: 118 | SEQ ID NO: 41 |
| SEQ ID NO: 119 | SEQ ID NO: 118 | SEQ ID NO: 43 |
| SEQ ID NO: 120 | SEQ ID NO: 118 | SEQ ID NO: 43 |
| SEQ ID NO: 121 | SEQ ID NO: 118 | SEQ ID NO: 43 |
| SEQ ID NO: 122 | SEQ ID NO: 118 | SEQ ID NO: 43 |
| SEQ ID NO: 123 | SEQ ID NO: 118 | SEQ ID NO: 43 |
| SEQ ID NO: 124 | SEQ ID NO: 118 | SEQ ID NO: 43 |
| SEQ ID NO: 125 | SEQ ID NO: 118 | SEQ ID NO: 43 |
| SEQ ID NO: 126 | SEQ ID NO: 118 | SEQ ID NO: 43 |
| SEQ ID NO: 127 | SEQ ID NO: 118 | SEQ ID NO: 43 |
| SEQ ID NO: 128 | SEQ ID NO: 118 | SEQ ID NO: 43 |
| SEQ ID NO: 129 | SEQ ID NO: 118 | SEQ ID NO: 43 |
| SEQ ID NO: 130 | SEQ ID NO: 118 | SEQ ID NO: 43 |
| SEQ ID NO: 131 | SEQ ID NO: 118 | SEQ ID NO: 43 |
| SEQ ID NO: 132 | SEQ ID NO: 118 | SEQ ID NO: 43 |
| SEQ ID NO: 133 | SEQ ID NO: 118 | SEQ ID NO: 43 |
| SEQ ID NO: 134 | SEQ ID NO: 118 | SEQ ID NO: 43 |
| SEQ ID NO: 135 | SEQ ID NO: 118 | SEQ ID NO: 43 |
| SEQ ID NO: 136 | SEQ ID NO: 118 | SEQ ID NO: 43 |
| SEQ ID NO: 137 | SEQ ID NO: 118 | SEQ ID NO: 43 |
| SEQ ID NO: 138 | SEQ ID NO: 118 | SEQ ID NO: 43 |
| SEQ ID NO: 139 | SEQ ID NO: 118 | SEQ ID NO: 43 |
| SEQ ID NO: 140 | SEQ ID NO: 118 | SEQ ID NO: 43 |
| SEQ ID NO: 141 | SEQ ID NO: 118 | SEQ ID NO: 43 |
| SEQ ID NO: 142 | SEQ ID NO: 118 | SEQ ID NO: 43 |
| SEQ ID NO: 143 | SEQ ID NO: 118 | SEQ ID NO: 43 |
| SEQ ID NO: 144 | SEQ ID NO: 118 | SEQ ID NO: 43 |
| SEQ ID NO: 145 | SEQ ID NO: 118 | SEQ ID NO: 43 |
| SEQ ID NO: 146 | SEQ ID NO: 118 | SEQ ID NO: 43 |
| SEQ ID NO: 147 | SEQ ID NO: 118 | SEQ ID NO: 43 |
| SEQ ID NO: 148 | SEQ ID NO: 118 | SEQ ID NO: 43 |
| SEQ ID NO: 149 | SEQ ID NO: 118 | SEQ ID NO: 43 |
| SEQ ID NO: 150 | SEQ ID NO: 118 | SEQ ID NO: 43 |
| SEQ ID NO: 151 | SEQ ID NO: 118 | SEQ ID NO: 43 |
| SEQ ID NO: 152 | SEQ ID NO: 118 | SEQ ID NO: 43 |
| SEQ ID NO: 153 | SEQ ID NO: 118 | SEQ ID NO: 43 |
| SEQ ID NO: 154 | SEQ ID NO: 118 | SEQ ID NO: 43 |
| SEQ ID NO: 155 | SEQ ID NO: 118 | SEQ ID NO: 43 |
| SEQ ID NO: 156 | SEQ ID NO: 118 | SEQ ID NO: 43 |
| SEQ ID NO: 157 | SEQ ID NO: 118 | SEQ ID NO: 43 |
| SEQ ID NO: 158 | SEQ ID NO: 118 | SEQ ID NO: 43 |
| SEQ ID NO: 159 | SEQ ID NO: 118 | SEQ ID NO: 43 |
| SEQ ID NO: 160 | SEQ ID NO: 118 | SEQ ID NO: 43 |
| SEQ ID NO: 161 | SEQ ID NO: 118 | SEQ ID NO: 43 |
| SEQ ID NO: 162 | SEQ ID NO: 118 | SEQ ID NO: 43 |
| SEQ ID NO: 163 | SEQ ID NO: 118 | SEQ ID NO: 43 |
| SEQ ID NO: 164 | SEQ ID NO: 118 | SEQ ID NO: 43 |
| SEQ ID NO: 165 | SEQ ID NO: 118 | SEQ ID NO: 43 |
| SEQ ID NO: 166 | SEQ ID NO: 118 | SEQ ID NO: 43 |
| SEQ ID NO: 167 | SEQ ID NO: 118 | SEQ ID NO: 43 |
| SEQ ID NO: 168 | SEQ ID NO: 118 | SEQ ID NO: 43 |
| SEQ ID NO: 169 | SEQ ID NO: 118 | SEQ ID NO: 43 |
| SEQ ID NO: 170 | SEQ ID NO: 118 | SEQ ID NO: 43 |
| SEQ ID NO: 171 | SEQ ID NO: 118 | SEQ ID NO: 43 |
| SEQ ID NO: 172 | SEQ ID NO: 118 | SEQ ID NO: 43 |
| SEQ ID NO: 173 | SEQ ID NO: 118 | SEQ ID NO: 43 |
| SEQ ID NO: 174 | SEQ ID NO: 118 | SEQ ID NO: 43 |
| SEQ ID NO: 175 | SEQ ID NO: 118 | SEQ ID NO: 43 |
| SEQ ID NO: 176 | SEQ ID NO: 118 | SEQ ID NO: 43 |
| SEQ ID NO: 177 | SEQ ID NO: 118 | SEQ ID NO: 43 |
| SEQ ID NO: 178 | SEQ ID NO: 118 | SEQ ID NO: 43 |
| SEQ ID NO: 179 | SEQ ID NO: 118 | SEQ ID NO: 43 |
| SEQ ID NO: 180 | SEQ ID NO: 118 | SEQ ID NO: 43 |
| SEQ ID NO: 181 | SEQ ID NO: 118 | SEQ ID NO: 43 |
| SEQ ID NO: 182 | SEQ ID NO: 118 | SEQ ID NO: 43 |
| SEQ ID NO: 183 | SEQ ID NO: 118 | SEQ ID NO: 43 |
| SEQ ID NO: 184 | SEQ ID NO: 118 | SEQ ID NO: 43 |
| SEQ ID NO: 185 | SEQ ID NO: 118 | SEQ ID NO: 43 |
| SEQ ID NO: 186 | SEQ ID NO: 118 | SEQ ID NO: 43 |
| SEQ ID NO: 187 | SEQ ID NO: 118 | SEQ ID NO: 43 |
| SEQ ID NO: 188 | SEQ ID NO: 118 | SEQ ID NO: 43 |
| SEQ ID NO: 189 | SEQ ID NO: 118 | SEQ ID NO: 43 |
| SEQ ID NO: 190 | SEQ ID NO: 118 | SEQ ID NO: 43 |
| SEQ ID NO: 191 | SEQ ID NO: 118 | SEQ ID NO: 43 |
| SEQ ID NO: 192 | SEQ ID NO: 118 | SEQ ID NO: 43 |
| SEQ ID NO: 193 | SEQ ID NO: 118 | SEQ ID NO: 43 |
| SEQ ID NO: 194 | SEQ ID NO: 118 | SEQ ID NO: 43 |
| SEQ ID NO: 195 | SEQ ID NO: 118 | SEQ ID NO: 43 |
| SEQ ID NO: 196 | SEQ ID NO: 118 | SEQ ID NO: 43 |
| SEQ ID NO: 197 | SEQ ID NO: 118 | SEQ ID NO: 43 |
| SEQ ID NO: 198 | SEQ ID NO: 118 | SEQ ID NO: 43 |
| SEQ ID NO: 199 | SEQ ID NO: 118 | SEQ ID NO: 43 |
| SEQ ID NO: 200 | SEQ ID NO: 118 | SEQ ID NO: 43 |
| SEQ ID NO: 201 | SEQ ID NO: 118 | SEQ ID NO: 43 |
| SEQ ID NO: 202 | SEQ ID NO: 118 | SEQ ID NO: 43 |
| SEQ ID NO: 203 | SEQ ID NO: 118 | SEQ ID NO: 43 |
| SEQ ID NO: 204 | SEQ ID NO: 118 | SEQ ID NO: 43 |
| SEQ ID NO: 205 | SEQ ID NO: 118 | SEQ ID NO: 43 |
| SEQ ID NO: 206 | SEQ ID NO: 118 | SEQ ID NO: 43 |
| SEQ ID NO: 207 | SEQ ID NO: 118 | SEQ ID NO: 43 |
| SEQ ID NO: 208 | SEQ ID NO: 118 | SEQ ID NO: 43 |
| SEQ ID NO: 209 | SEQ ID NO: 118 | SEQ ID NO: 43 |
| SEQ ID NO: 210 | SEQ ID NO: 118 | SEQ ID NO: 43 |
| SEQ ID NO: 211 | SEQ ID NO: 118 | SEQ ID NO: 43 |
| SEQ ID NO: 212 | SEQ ID NO: 118 | SEQ ID NO: 43 |
| SEQ ID NO: 213 | SEQ ID NO: 118 | SEQ ID NO: 43 |
| SEQ ID NO: 214 | SEQ ID NO: 118 | SEQ ID NO: 43 |
| SEQ ID NO: 215 | SEQ ID NO: 118 | SEQ ID NO: 43 |
| SEQ ID NO: 216 | SEQ ID NO: 118 | SEQ ID NO: 43 |
| SEQ ID NO: 217 | SEQ ID NO: 118 | SEQ ID NO: 43 |
| SEQ ID NO: 218 | SEQ ID NO: 118 | SEQ ID NO: 43 |
| SEQ ID NO: 219 | SEQ ID NO: 118 | SEQ ID NO: 43 |
| SEQ ID NO: 220 | SEQ ID NO: 118 | SEQ ID NO: 43 |
| SEQ ID NO: 221 | SEQ ID NO: 118 | SEQ ID NO: 43 |
| SEQ ID NO: 329 | SEQ ID NO: 118 | SEQ ID NO: 43 |
| SEQ ID NO: 332 | SEQ ID NO: 118 | SEQ ID NO: 43 |
| SEQ ID NO: 335 | SEQ ID NO: 118 | SEQ ID NO: 43 |

TABLE 5
Example configurations of fusion proteins
Components (from an N-terminus to C-terminus direction)

| Heterologous | | Mutant Soluble |
|---|---|---|
| Protein | Linker(s) | ActRIIB-ECD polypeptide |
| SEQ ID NO: 39 | SEQ ID NOs: 118 and 44 | SEQ ID NO: 119 |
| SEQ ID NO: 39 | SEQ ID NOs: 118 and 44 | SEQ ID NO: 120 |
| SEQ ID NO: 39 | SEQ ID NOs: 118 and 44 | SEQ ID NO: 121 |
| SEQ ID NO: 39 | SEQ ID NOs: 118 and 44 | SEQ ID NO: 122 |
| SEQ ID NO: 39 | SEQ ID NOs: 118 and 44 | SEQ ID NO: 123 |
| SEQ ID NO: 39 | SEQ ID NOs: 118 and 44 | SEQ ID NO: 124 |
| SEQ ID NO: 39 | SEQ ID NOs: 118 and 44 | SEQ ID NO: 125 |
| SEQ ID NO: 39 | SEQ ID NOs: 118 and 44 | SEQ ID NO: 126 |
| SEQ ID NO: 39 | SEQ ID NOs: 118 and 44 | SEQ ID NO: 127 |
| SEQ ID NO: 39 | SEQ ID NOs: 118 and 44 | SEQ ID NO: 128 |
| SEQ ID NO: 39 | SEQ ID NOs: 118 and 44 | SEQ ID NO: 129 |
| SEQ ID NO: 39 | SEQ ID NOs: 118 and 44 | SEQ ID NO: 130 |
| SEQ ID NO: 39 | SEQ ID NOs: 118 and 44 | SEQ ID NO: 131 |
| SEQ ID NO: 39 | SEQ ID NOs: 118 and 44 | SEQ ID NO: 132 |
| SEQ ID NO: 39 | SEQ ID NOs: 118 and 44 | SEQ ID NO: 133 |
| SEQ ID NO: 39 | SEQ ID NOs: 118 and 44 | SEQ ID NO: 134 |
| SEQ ID NO: 39 | SEQ ID NOs: 118 and 44 | SEQ ID NO: 135 |
| SEQ ID NO: 39 | SEQ ID NOs: 118 and 44 | SEQ ID NO: 136 |
| SEQ ID NO: 39 | SEQ ID NOs: 118 and 44 | SEQ ID NO: 137 |
| SEQ ID NO: 39 | SEQ ID NOs: 118 and 44 | SEQ ID NO: 138 |
| SEQ ID NO: 39 | SEQ ID NOs: 118 and 44 | SEQ ID NO: 139 |
| SEQ ID NO: 39 | SEQ ID NOs: 118 and 44 | SEQ ID NO: 140 |
| SEQ ID NO: 39 | SEQ ID NOs: 118 and 44 | SEQ ID NO: 141 |
| SEQ ID NO: 39 | SEQ ID NOs: 118 and 44 | SEQ ID NO: 142 |
| SEQ ID NO: 39 | SEQ ID NOs: 118 and 44 | SEQ ID NO: 143 |
| SEQ ID NO: 39 | SEQ ID NOs: 118 and 44 | SEQ ID NO: 144 |
| SEQ ID NO: 39 | SEQ ID NOs: 118 and 44 | SEQ ID NO: 145 |
| SEQ ID NO: 39 | SEQ ID NOs: 118 and 44 | SEQ ID NO: 146 |
| SEQ ID NO: 39 | SEQ ID NOs: 118 and 44 | SEQ ID NO: 147 |
| SEQ ID NO: 39 | SEQ ID NOs: 118 and 44 | SEQ ID NO: 148 |
| SEQ ID NO: 39 | SEQ ID NOs: 118 and 44 | SEQ ID NO: 149 |
| SEQ ID NO: 39 | SEQ ID NOs: 118 and 44 | SEQ ID NO: 150 |
| SEQ ID NO: 39 | SEQ ID NOs: 118 and 44 | SEQ ID NO: 151 |
| SEQ ID NO: 39 | SEQ ID NOs: 118 and 44 | SEQ ID NO: 152 |
| SEQ ID NO: 39 | SEQ ID NOs: 118 and 44 | SEQ ID NO: 153 |
| SEQ ID NO: 39 | SEQ ID NOs: 118 and 44 | SEQ ID NO: 154 |
| SEQ ID NO: 39 | SEQ ID NOs: 118 and 44 | SEQ ID NO: 155 |
| SEQ ID NO: 39 | SEQ ID NOs: 118 and 44 | SEQ ID NO: 156 |
| SEQ ID NO: 39 | SEQ ID NOs: 118 and 44 | SEQ ID NO: 157 |
| SEQ ID NO: 39 | SEQ ID NOs: 118 and 44 | SEQ ID NO: 158 |
| SEQ ID NO: 39 | SEQ ID NOs: 118 and 44 | SEQ ID NO: 159 |
| SEQ ID NO: 39 | SEQ ID NOs: 118 and 44 | SEQ ID NO: 160 |
| SEQ ID NO: 39 | SEQ ID NOs: 118 and 44 | SEQ ID NO: 161 |
| SEQ ID NO: 39 | SEQ ID NOs: 118 and 44 | SEQ ID NO: 162 |
| SEQ ID NO: 39 | SEQ ID NOs: 118 and 44 | SEQ ID NO: 163 |
| SEQ ID NO: 39 | SEQ ID NOs: 118 and 44 | SEQ ID NO: 164 |
| SEQ ID NO: 39 | SEQ ID NOs: 118 and 44 | SEQ ID NO: 165 |
| SEQ ID NO: 39 | SEQ ID NOs: 118 and 44 | SEQ ID NO: 166 |
| SEQ ID NO: 39 | SEQ ID NOs: 118 and 44 | SEQ ID NO: 167 |
| SEQ ID NO: 39 | SEQ ID NOs: 118 and 44 | SEQ ID NO: 168 |
| SEQ ID NO: 39 | SEQ ID NOs: 118 and 44 | SEQ ID NO: 169 |
| SEQ ID NO: 39 | SEQ ID NOs: 118 and 44 | SEQ ID NO: 170 |
| SEQ ID NO: 39 | SEQ ID NOs: 118 and 44 | SEQ ID NO: 171 |
| SEQ ID NO: 39 | SEQ ID NOs: 118 and 44 | SEQ ID NO: 172 |
| SEQ ID NO: 39 | SEQ ID NOs: 118 and 44 | SEQ ID NO: 173 |
| SEQ ID NO: 39 | SEQ ID NOs: 118 and 44 | SEQ ID NO: 174 |
| SEQ ID NO: 39 | SEQ ID NOs: 118 and 44 | SEQ ID NO: 175 |
| SEQ ID NO: 39 | SEQ ID NOs: 118 and 44 | SEQ ID NO: 176 |
| SEQ ID NO: 39 | SEQ ID NOs: 118 and 44 | SEQ ID NO: 177 |
| SEQ ID NO: 39 | SEQ ID NOs: 118 and 44 | SEQ ID NO: 178 |
| SEQ ID NO: 39 | SEQ ID NOs: 118 and 44 | SEQ ID NO: 179 |
| SEQ ID NO: 39 | SEQ ID NOs: 118 and 44 | SEQ ID NO: 180 |
| SEQ ID NO: 39 | SEQ ID NOs: 118 and 44 | SEQ ID NO: 181 |
| SEQ ID NO: 39 | SEQ ID NOs: 118 and 44 | SEQ ID NO: 182 |
| SEQ ID NO: 39 | SEQ ID NOs: 118 and 44 | SEQ ID NO: 183 |
| SEQ ID NO: 39 | SEQ ID NOs: 118 and 44 | SEQ ID NO: 184 |
| SEQ ID NO: 39 | SEQ ID NOs: 118 and 44 | SEQ ID NO: 185 |
| SEQ ID NO: 39 | SEQ ID NOs: 118 and 44 | SEQ ID NO: 186 |
| SEQ ID NO: 39 | SEQ ID NOs: 118 and 44 | SEQ ID NO: 187 |
| SEQ ID NO: 39 | SEQ ID NOs: 118 and 44 | SEQ ID NO: 188 |
| SEQ ID NO: 39 | SEQ ID NOs: 118 and 44 | SEQ ID NO: 189 |
| SEQ ID NO: 39 | SEQ ID NOs: 118 and 44 | SEQ ID NO: 190 |
| SEQ ID NO: 39 | SEQ ID NOs: 118 and 44 | SEQ ID NO: 191 |
| SEQ ID NO: 39 | SEQ ID NOs: 118 and 44 | SEQ ID NO: 192 |
| SEQ ID NO: 39 | SEQ ID NOs: 118 and 44 | SEQ ID NO: 193 |
| SEQ ID NO: 39 | SEQ ID NOs: 118 and 44 | SEQ ID NO: 194 |
| SEQ ID NO: 39 | SEQ ID NOs: 118 and 44 | SEQ ID NO: 195 |
| SEQ ID NO: 39 | SEQ ID NOs: 118 and 44 | SEQ ID NO: 196 |
| SEQ ID NO: 39 | SEQ ID NOs: 118 and 44 | SEQ ID NO: 197 |
| SEQ ID NO: 39 | SEQ ID NOs: 118 and 44 | SEQ ID NO: 198 |
| SEQ ID NO: 39 | SEQ ID NOs: 118 and 44 | SEQ ID NO: 199 |
| SEQ ID NO: 39 | SEQ ID NOs: 118 and 44 | SEQ ID NO: 200 |
| SEQ ID NO: 39 | SEQ ID NOs: 118 and 44 | SEQ ID NO: 201 |
| SEQ ID NO: 39 | SEQ ID NOs: 118 and 44 | SEQ ID NO: 202 |
| SEQ ID NO: 39 | SEQ ID NOs: 118 and 44 | SEQ ID NO: 203 |
| SEQ ID NO: 39 | SEQ ID NOs: 118 and 44 | SEQ ID NO: 204 |
| SEQ ID NO: 39 | SEQ ID NOs: 118 and 44 | SEQ ID NO: 205 |
| SEQ ID NO: 39 | SEQ ID NOs: 118 and 44 | SEQ ID NO: 206 |
| SEQ ID NO: 39 | SEQ ID NOs: 118 and 44 | SEQ ID NO: 207 |
| SEQ ID NO: 39 | SEQ ID NOs: 118 and 44 | SEQ ID NO: 208 |
| SEQ ID NO: 39 | SEQ ID NOs: 118 and 44 | SEQ ID NO: 209 |
| SEQ ID NO: 39 | SEQ ID NOs: 118 and 44 | SEQ ID NO: 210 |
| SEQ ID NO: 39 | SEQ ID NOs: 118 and 44 | SEQ ID NO: 211 |
| SEQ ID NO: 39 | SEQ ID NOs: 118 and 44 | SEQ ID NO: 212 |
| SEQ ID NO: 39 | SEQ ID NOs: 118 and 44 | SEQ ID NO: 213 |
| SEQ ID NO: 39 | SEQ ID NOs: 118 and 44 | SEQ ID NO: 214 |
| SEQ ID NO: 39 | SEQ ID NOs: 118 and 44 | SEQ ID NO: 215 |
| SEQ ID NO: 39 | SEQ ID NOs: 118 and 44 | SEQ ID NO: 216 |
| SEQ ID NO: 39 | SEQ ID NOs: 118 and 44 | SEQ ID NO: 217 |
| SEQ ID NO: 39 | SEQ ID NOs: 118 and 44 | SEQ ID NO: 218 |
| SEQ ID NO: 39 | SEQ ID NOs: 118 and 44 | SEQ ID NO: 219 |
| SEQ ID NO: 39 | SEQ ID NOs: 118 and 44 | SEQ ID NO: 220 |
| SEQ ID NO: 39 | SEQ ID NOs: 118 and 44 | SEQ ID NO: 221 |
| SEQ ID NO: 41 | SEQ ID NOs: 118 and 44 | SEQ ID NO: 120 |
| SEQ ID NO: 41 | SEQ ID NOs: 118 and 44 | SEQ ID NO: 121 |
| SEQ ID NO: 41 | SEQ ID NOs: 118 and 44 | SEQ ID NO: 122 |
| SEQ ID NO: 41 | SEQ ID NOs: 118 and 44 | SEQ ID NO: 123 |
| SEQ ID NO: 41 | SEQ ID NOs: 118 and 44 | SEQ ID NO: 124 |
| SEQ ID NO: 41 | SEQ ID NOs: 118 and 44 | SEQ ID NO: 125 |
| SEQ ID NO: 41 | SEQ ID NOs: 118 and 44 | SEQ ID NO: 126 |
| SEQ ID NO: 41 | SEQ ID NOs: 118 and 44 | SEQ ID NO: 127 |
| SEQ ID NO: 41 | SEQ ID NOs: 118 and 44 | SEQ ID NO: 128 |
| SEQ ID NO: 41 | SEQ ID NOs: 118 and 44 | SEQ ID NO: 129 |
| SEQ ID NO: 41 | SEQ ID NOs: 118 and 44 | SEQ ID NO: 130 |
| SEQ ID NO: 41 | SEQ ID NOs: 118 and 44 | SEQ ID NO: 131 |
| SEQ ID NO: 41 | SEQ ID NOs: 118 and 44 | SEQ ID NO: 132 |
| SEQ ID NO: 41 | SEQ ID NOs: 118 and 44 | SEQ ID NO: 133 |
| SEQ ID NO: 41 | SEQ ID NOs: 118 and 44 | SEQ ID NO: 134 |
| SEQ ID NO: 41 | SEQ ID NOs: 118 and 44 | SEQ ID NO: 135 |
| SEQ ID NO: 41 | SEQ ID NOs: 118 and 44 | SEQ ID NO: 136 |
| SEQ ID NO: 41 | SEQ ID NOs: 118 and 44 | SEQ ID NO: 137 |
| SEQ ID NO: 41 | SEQ ID NOs: 118 and 44 | SEQ ID NO: 138 |
| SEQ ID NO: 41 | SEQ ID NOs: 118 and 44 | SEQ ID NO: 139 |
| SEQ ID NO: 41 | SEQ ID NOs: 118 and 44 | SEQ ID NO: 140 |
| SEQ ID NO: 41 | SEQ ID NOs: 118 and 44 | SEQ ID NO: 141 |
| SEQ ID NO: 41 | SEQ ID NOs: 118 and 44 | SEQ ID NO: 142 |
| SEQ ID NO: 41 | SEQ ID NOs: 118 and 44 | SEQ ID NO: 143 |
| SEQ ID NO: 41 | SEQ ID NOs: 118 and 44 | SEQ ID NO: 144 |
| SEQ ID NO: 41 | SEQ ID NOs: 118 and 44 | SEQ ID NO: 145 |
| SEQ ID NO: 41 | SEQ ID NOs: 118 and 44 | SEQ ID NO: 146 |
| SEQ ID NO: 41 | SEQ ID NOs: 118 and 44 | SEQ ID NO: 147 |
| SEQ ID NO: 41 | SEQ ID NOs: 118 and 44 | SEQ ID NO: 148 |
| SEQ ID NO: 41 | SEQ ID NOs: 118 and 44 | SEQ ID NO: 149 |
| SEQ ID NO: 41 | SEQ ID NOs: 118 and 44 | SEQ ID NO: 150 |
| SEQ ID NO: 41 | SEQ ID NOs: 118 and 44 | SEQ ID NO: 151 |
| SEQ ID NO: 41 | SEQ ID NOs: 118 and 44 | SEQ ID NO: 152 |
| SEQ ID NO: 41 | SEQ ID NOs: 118 and 44 | SEQ ID NO: 153 |
| SEQ ID NO: 41 | SEQ ID NOs: 118 and 44 | SEQ ID NO: 154 |
| SEQ ID NO: 41 | SEQ ID NOs: 118 and 44 | SEQ ID NO: 155 |
| SEQ ID NO: 41 | SEQ ID NOs: 118 and 44 | SEQ ID NO: 156 |
| SEQ ID NO: 41 | SEQ ID NOs: 118 and 44 | SEQ ID NO: 157 |
| SEQ ID NO: 41 | SEQ ID NOs: 118 and 44 | SEQ ID NO: 158 |
| SEQ ID NO: 41 | SEQ ID NOs: 118 and 44 | SEQ ID NO: 159 |
| SEQ ID NO: 41 | SEQ ID NOs: 118 and 44 | SEQ ID NO: 160 |
| SEQ ID NO: 41 | SEQ ID NOs: 118 and 44 | SEQ ID NO: 161 |
| SEQ ID NO: 41 | SEQ ID NOs: 118 and 44 | SEQ ID NO: 162 |
| SEQ ID NO: 41 | SEQ ID NOs: 118 and 44 | SEQ ID NO: 163 |
| SEQ ID NO: 41 | SEQ ID NOs: 118 and 44 | SEQ ID NO: 164 |
| SEQ ID NO: 41 | SEQ ID NOs: 118 and 44 | SEQ ID NO: 165 |
| SEQ ID NO: 41 | SEQ ID NOs: 118 and 44 | SEQ ID NO: 166 |
| SEQ ID NO: 41 | SEQ ID NOs: 118 and 44 | SEQ ID NO: 167 |
| SEQ ID NO: 41 | SEQ ID NOs: 118 and 44 | SEQ ID NO: 168 |
| SEQ ID NO: 41 | SEQ ID NOs: 118 and 44 | SEQ ID NO: 169 |
| SEQ ID NO: 41 | SEQ ID NOs: 118 and 44 | SEQ ID NO: 170 |
| SEQ ID NO: 41 | SEQ ID NOs: 118 and 44 | SEQ ID NO: 171 |
| SEQ ID NO: 41 | SEQ ID NOs: 118 and 44 | SEQ ID NO: 172 |
| SEQ ID NO: 41 | SEQ ID NOs: 118 and 44 | SEQ ID NO: 173 |
| SEQ ID NO: 41 | SEQ ID NOs: 118 and 44 | SEQ ID NO: 174 |
| SEQ ID NO: 41 | SEQ ID NOs: 118 and 44 | SEQ ID NO: 175 |
| SEQ ID NO: 41 | SEQ ID NOs: 118 and 44 | SEQ ID NO: 176 |
| SEQ ID NO: 41 | SEQ ID NOs: 118 and 44 | SEQ ID NO: 177 |
| SEQ ID NO: 41 | SEQ ID NOs: 118 and 44 | SEQ ID NO: 178 |
| SEQ ID NO: 41 | SEQ ID NOs: 118 and 44 | SEQ ID NO: 179 |
| SEQ ID NO: 41 | SEQ ID NOs: 118 and 44 | SEQ ID NO: 180 |
| SEQ ID NO: 41 | SEQ ID NOs: 118 and 44 | SEQ ID NO: 181 |
| SEQ ID NO: 41 | SEQ ID NOs: 118 and 44 | SEQ ID NO: 182 |
| SEQ ID NO: 41 | SEQ ID NOs: 118 and 44 | SEQ ID NO: 183 |
| SEQ ID NO: 41 | SEQ ID NOs: 118 and 44 | SEQ ID NO: 184 |
| SEQ ID NO: 41 | SEQ ID NOs: 118 and 44 | SEQ ID NO: 185 |
| SEQ ID NO: 41 | SEQ ID NOs: 118 and 44 | SEQ ID NO: 186 |
| SEQ ID NO: 41 | SEQ ID NOs: 118 and 44 | SEQ ID NO: 187 |
| SEQ ID NO: 41 | SEQ ID NOs: 118 and 44 | SEQ ID NO: 188 |
| SEQ ID NO: 41 | SEQ ID NOs: 118 and 44 | SEQ ID NO: 189 |
| SEQ ID NO: 41 | SEQ ID NOs: 118 and 44 | SEQ ID NO: 190 |
| SEQ ID NO: 41 | SEQ ID NOs: 118 and 44 | SEQ ID NO: 191 |
| SEQ ID NO: 41 | SEQ ID NOs: 118 and 44 | SEQ ID NO: 192 |
| SEQ ID NO: 41 | SEQ ID NOs: 118 and 44 | SEQ ID NO: 193 |
| SEQ ID NO: 41 | SEQ ID NOs: 118 and 44 | SEQ ID NO: 194 |
| SEQ ID NO: 41 | SEQ ID NOs: 118 and 44 | SEQ ID NO: 195 |
| SEQ ID NO: 41 | SEQ ID NOs: 118 and 44 | SEQ ID NO: 196 |
| SEQ ID NO: 41 | SEQ ID NOs: 118 and 44 | SEQ ID NO: 197 |
| SEQ ID NO: 41 | SEQ ID NOs: 118 and 44 | SEQ ID NO: 198 |
| SEQ ID NO: 41 | SEQ ID NOs: 118 and 44 | SEQ ID NO: 199 |
| SEQ ID NO: 41 | SEQ ID NOs: 118 and 44 | SEQ ID NO: 200 |
| SEQ ID NO: 41 | SEQ ID NOs: 118 and 44 | SEQ ID NO: 201 |
| SEQ ID NO: 41 | SEQ ID NOs: 118 and 44 | SEQ ID NO: 202 |
| SEQ ID NO: 41 | SEQ ID NOs: 118 and 44 | SEQ ID NO: 203 |
| SEQ ID NO: 41 | SEQ ID NOs: 118 and 44 | SEQ ID NO: 204 |
| SEQ ID NO: 41 | SEQ ID NOs: 118 and 44 | SEQ ID NO: 205 |
| SEQ ID NO: 41 | SEQ ID NOs: 118 and 44 | SEQ ID NO: 206 |
| SEQ ID NO: 41 | SEQ ID NOs: 118 and 44 | SEQ ID NO: 207 |
| SEQ ID NO: 41 | SEQ ID NOs: 118 and 44 | SEQ ID NO: 208 |
| SEQ ID NO: 41 | SEQ ID NOs: 118 and 44 | SEQ ID NO: 209 |
| SEQ ID NO: 41 | SEQ ID NOs: 118 and 44 | SEQ ID NO: 210 |
| SEQ ID NO: 41 | SEQ ID NOs: 118 and 44 | SEQ ID NO: 211 |
| SEQ ID NO: 41 | SEQ ID NOs: 118 and 44 | SEQ ID NO: 212 |
| SEQ ID NO: 41 | SEQ ID NOs: 118 and 44 | SEQ ID NO: 213 |
| SEQ ID NO: 41 | SEQ ID NOs: 118 and 44 | SEQ ID NO: 214 |
| SEQ ID NO: 41 | SEQ ID NOs: 118 and 44 | SEQ ID NO: 215 |
| SEQ ID NO: 41 | SEQ ID NOs: 118 and 44 | SEQ ID NO: 216 |
| SEQ ID NO: 41 | SEQ ID NOs: 118 and 44 | SEQ ID NO: 217 |
| SEQ ID NO: 41 | SEQ ID NOs: 118 and 44 | SEQ ID NO: 218 |
| SEQ ID NO: 41 | SEQ ID NOs: 118 and 44 | SEQ ID NO: 219 |
| SEQ ID NO: 41 | SEQ ID NOs: 118 and 44 | SEQ ID NO: 220 |
| SEQ ID NO: 41 | SEQ ID NOs: 118 and 44 | SEQ ID NO: 221 |
| SEQ ID NO: 43 | SEQ ID NOs: 118 and 44 | SEQ ID NO: 120 |
| SEQ ID NO: 43 | SEQ ID NOs: 118 and 44 | SEQ ID NO: 121 |
| SEQ ID NO: 43 | SEQ ID NOs: 118 and 44 | SEQ ID NO: 122 |
| SEQ ID NO: 43 | SEQ ID NOs: 118 and 44 | SEQ ID NO: 123 |
| SEQ ID NO: 43 | SEQ ID NOs: 118 and 44 | SEQ ID NO: 124 |
| SEQ ID NO: 43 | SEQ ID NOs: 118 and 44 | SEQ ID NO: 125 |
| SEQ ID NO: 43 | SEQ ID NOs: 118 and 44 | SEQ ID NO: 126 |
| SEQ ID NO: 43 | SEQ ID NOs: 118 and 44 | SEQ ID NO: 127 |
| SEQ ID NO: 43 | SEQ ID NOs: 118 and 44 | SEQ ID NO: 128 |
| SEQ ID NO: 43 | SEQ ID NOs: 118 and 44 | SEQ ID NO: 129 |
| SEQ ID NO: 43 | SEQ ID NOs: 118 and 44 | SEQ ID NO: 130 |
| SEQ ID NO: 43 | SEQ ID NOs: 118 and 44 | SEQ ID NO: 131 |
| SEQ ID NO: 43 | SEQ ID NOs: 118 and 44 | SEQ ID NO: 132 |
| SEQ ID NO: 43 | SEQ ID NOs: 118 and 44 | SEQ ID NO: 133 |
| SEQ ID NO: 43 | SEQ ID NOs: 118 and 44 | SEQ ID NO: 134 |
| SEQ ID NO: 43 | SEQ ID NOs: 118 and 44 | SEQ ID NO: 135 |
| SEQ ID NO: 43 | SEQ ID NOs: 118 and 44 | SEQ ID NO: 136 |
| SEQ ID NO: 43 | SEQ ID NOs: 118 and 44 | SEQ ID NO: 137 |
| SEQ ID NO: 43 | SEQ ID NOs: 118 and 44 | SEQ ID NO: 138 |
| SEQ ID NO: 43 | SEQ ID NOs: 118 and 44 | SEQ ID NO: 139 |
| SEQ ID NO: 43 | SEQ ID NOs: 118 and 44 | SEQ ID NO: 140 |
| SEQ ID NO: 43 | SEQ ID NOs: 118 and 44 | SEQ ID NO: 141 |
| SEQ ID NO: 43 | SEQ ID NOs: 118 and 44 | SEQ ID NO: 142 |
| SEQ ID NO: 43 | SEQ ID NOs: 118 and 44 | SEQ ID NO: 143 |
| SEQ ID NO: 43 | SEQ ID NOs: 118 and 44 | SEQ ID NO: 144 |
| SEQ ID NO: 43 | SEQ ID NOs: 118 and 44 | SEQ ID NO: 145 |
| SEQ ID NO: 43 | SEQ ID NOs: 118 and 44 | SEQ ID NO: 146 |
| SEQ ID NO: 43 | SEQ ID NOs: 118 and 44 | SEQ ID NO: 147 |
| SEQ ID NO: 43 | SEQ ID NOs: 118 and 44 | SEQ ID NO: 148 |
| SEQ ID NO: 43 | SEQ ID NOs: 118 and 44 | SEQ ID NO: 149 |
| SEQ ID NO: 43 | SEQ ID NOs: 118 and 44 | SEQ ID NO: 150 |
| SEQ ID NO: 43 | SEQ ID NOs: 118 and 44 | SEQ ID NO: 151 |
| SEQ ID NO: 43 | SEQ ID NOs: 118 and 44 | SEQ ID NO: 152 |
| SEQ ID NO: 43 | SEQ ID NOs: 118 and 44 | SEQ ID NO: 153 |
| SEQ ID NO: 43 | SEQ ID NOs: 118 and 44 | SEQ ID NO: 154 |
| SEQ ID NO: 43 | SEQ ID NOs: 118 and 44 | SEQ ID NO: 155 |
| SEQ ID NO: 43 | SEQ ID NOs: 118 and 44 | SEQ ID NO: 156 |
| SEQ ID NO: 43 | SEQ ID NOs: 118 and 44 | SEQ ID NO: 157 |
| SEQ ID NO: 43 | SEQ ID NOs: 118 and 44 | SEQ ID NO: 158 |
| SEQ ID NO: 43 | SEQ ID NOs: 118 and 44 | SEQ ID NO: 159 |
| SEQ ID NO: 43 | SEQ ID NOs: 118 and 44 | SEQ ID NO: 160 |
| SEQ ID NO: 43 | SEQ ID NOs: 118 and 44 | SEQ ID NO: 161 |
| SEQ ID NO: 43 | SEQ ID NOs: 118 and 44 | SEQ ID NO: 162 |
| SEQ ID NO: 43 | SEQ ID NOs: 118 and 44 | SEQ ID NO: 163 |
| SEQ ID NO: 43 | SEQ ID NOs: 118 and 44 | SEQ ID NO: 164 |
| SEQ ID NO: 43 | SEQ ID NOs: 118 and 44 | SEQ ID NO: 165 |
| SEQ ID NO: 43 | SEQ ID NOs: 118 and 44 | SEQ ID NO: 166 |
| SEQ ID NO: 43 | SEQ ID NOs: 118 and 44 | SEQ ID NO: 167 |
| SEQ ID NO: 43 | SEQ ID NOs: 118 and 44 | SEQ ID NO: 168 |
| SEQ ID NO: 43 | SEQ ID NOs: 118 and 44 | SEQ ID NO: 169 |
| SEQ ID NO: 43 | SEQ ID NOs: 118 and 44 | SEQ ID NO: 170 |
| SEQ ID NO: 43 | SEQ ID NOs: 118 and 44 | SEQ ID NO: 171 |
| SEQ ID NO: 43 | SEQ ID NOs: 118 and 44 | SEQ ID NO: 172 |
| SEQ ID NO: 43 | SEQ ID NOs: 118 and 44 | SEQ ID NO: 173 |
| SEQ ID NO: 43 | SEQ ID NOs: 118 and 44 | SEQ ID NO: 174 |
| SEQ ID NO: 43 | SEQ ID NOs: 118 and 44 | SEQ ID NO: 175 |
| SEQ ID NO: 43 | SEQ ID NOs: 118 and 44 | SEQ ID NO: 176 |
| SEQ ID NO: 43 | SEQ ID NOs: 118 and 44 | SEQ ID NO: 177 |
| SEQ ID NO: 43 | SEQ ID NOs: 118 and 44 | SEQ ID NO: 178 |
| SEQ ID NO: 43 | SEQ ID NOs: 118 and 44 | SEQ ID NO: 179 |
| SEQ ID NO: 43 | SEQ ID NOs: 118 and 44 | SEQ ID NO: 180 |
| SEQ ID NO: 43 | SEQ ID NOs: 118 and 44 | SEQ ID NO: 181 |
| SEQ ID NO: 43 | SEQ ID NOs: 118 and 44 | SEQ ID NO: 182 |
| SEQ ID NO: 43 | SEQ ID NOs: 118 and 44 | SEQ ID NO: 183 |
| SEQ ID NO: 43 | SEQ ID NOs: 118 and 44 | SEQ ID NO: 184 |
| SEQ ID NO: 43 | SEQ ID NOs: 118 and 44 | SEQ ID NO: 185 |
| SEQ ID NO: 43 | SEQ ID NOs: 118 and 44 | SEQ ID NO: 186 |
| SEQ ID NO: 43 | SEQ ID NOs: 118 and 44 | SEQ ID NO: 187 |
| SEQ ID NO: 43 | SEQ ID NOs: 118 and 44 | SEQ ID NO: 188 |
| SEQ ID NO: 43 | SEQ ID NOs: 118 and 44 | SEQ ID NO: 189 |
| SEQ ID NO: 43 | SEQ ID NOs: 118 and 44 | SEQ ID NO: 190 |
| SEQ ID NO: 43 | SEQ ID NOs: 118 and 44 | SEQ ID NO: 191 |
| SEQ ID NO: 43 | SEQ ID NOs: 118 and 44 | SEQ ID NO: 192 |
| SEQ ID NO: 43 | SEQ ID NOs: 118 and 44 | SEQ ID NO: 193 |
| SEQ ID NO: 43 | SEQ ID NOs: 118 and 44 | SEQ ID NO: 194 |
| SEQ ID NO: 43 | SEQ ID NOs: 118 and 44 | SEQ ID NO: 195 |
| SEQ ID NO: 43 | SEQ ID NOs: 118 and 44 | SEQ ID NO: 196 |
| SEQ ID NO: 43 | SEQ ID NOs: 118 and 44 | SEQ ID NO: 197 |
| SEQ ID NO: 43 | SEQ ID NOs: 118 and 44 | SEQ ID NO: 198 |
| SEQ ID NO: 43 | SEQ ID NOs: 118 and 44 | SEQ ID NO: 199 |
| SEQ ID NO: 43 | SEQ ID NOs: 118 and 44 | SEQ ID NO: 200 |
| SEQ ID NO: 43 | SEQ ID NOs: 118 and 44 | SEQ ID NO: 201 |
| SEQ ID NO: 43 | SEQ ID NOs: 118 and 44 | SEQ ID NO: 202 |
| SEQ ID NO: 43 | SEQ ID NOs: 118 and 44 | SEQ ID NO: 203 |
| SEQ ID NO: 43 | SEQ ID NOs: 118 and 44 | SEQ ID NO: 204 |
| SEQ ID NO: 43 | SEQ ID NOs: 118 and 44 | SEQ ID NO: 205 |
| SEQ ID NO: 43 | SEQ ID NOs: 118 and 44 | SEQ ID NO: 206 |
| SEQ ID NO: 43 | SEQ ID NOs: 118 and 44 | SEQ ID NO: 207 |
| SEQ ID NO: 43 | SEQ ID NOs: 118 and 44 | SEQ ID NO: 208 |
| SEQ ID NO: 43 | SEQ ID NOs: 118 and 44 | SEQ ID NO: 209 |
| SEQ ID NO: 43 | SEQ ID NOs: 118 and 44 | SEQ ID NO: 210 |
| SEQ ID NO: 43 | SEQ ID NOs: 118 and 44 | SEQ ID NO: 211 |
| SEQ ID NO: 43 | SEQ ID NOs: 118 and 44 | SEQ ID NO: 212 |
| SEQ ID NO: 43 | SEQ ID NOs: 118 and 44 | SEQ ID NO: 213 |
| SEQ ID NO: 43 | SEQ ID NOs: 118 and 44 | SEQ ID NO: 214 |
| SEQ ID NO: 43 | SEQ ID NOs: 118 and 44 | SEQ ID NO: 215 |
| SEQ ID NO: 43 | SEQ ID NOs: 118 and 44 | SEQ ID NO: 216 |
| SEQ ID NO: 43 | SEQ ID NOs: 118 and 44 | SEQ ID NO: 217 |
| SEQ ID NO: 43 | SEQ ID NOs: 118 and 44 | SEQ ID NO: 218 |
| SEQ ID NO: 43 | SEQ ID NOs: 118 and 44 | SEQ ID NO: 219 |
| SEQ ID NO: 43 | SEQ ID NOs: 118 and 44 | SEQ ID NO: 220 |
| SEQ ID NO: 43 | SEQ ID NOs: 118 and 44 | SEQ ID NO: 221 |
| SEQ ID NO: 43 | SEQ ID NOs: 118 and 44 | SEQ ID NO: 329 |
| SEQ ID NO: 43 | SEQ ID NOs: 118 and 44 | SEQ ID NO: 332 |
| SEQ ID NO: 43 | SEQ ID NOs: 118 and 44 | SEQ ID NO: 335 |

TABLE 6
Example configurations of fusion proteins
Components (from an N-terminus to C-terminus direction)

| Heterologous | | Mutant Soluble |
|---|---|---|
| Protein | Linker(s) | ActRIIB-ECD polypeptide |
| SEQ ID NO: 39 | SEQ ID NO: 44 | SEQ ID NO: 119 |
| SEQ ID NO: 39 | SEQ ID NO: 44 | SEQ ID NO: 120 |
| SEQ ID NO: 39 | SEQ ID NO: 44 | SEQ ID NO: 121 |
| SEQ ID NO: 39 | SEQ ID NO: 44 | SEQ ID NO: 122 |
| SEQ ID NO: 39 | SEQ ID NO: 44 | SEQ ID NO: 123 |
| SEQ ID NO: 39 | SEQ ID NO: 44 | SEQ ID NO: 124 |
| SEQ ID NO: 39 | SEQ ID NO: 44 | SEQ ID NO: 125 |
| SEQ ID NO: 39 | SEQ ID NO: 44 | SEQ ID NO: 126 |
| SEQ ID NO: 39 | SEQ ID NO: 44 | SEQ ID NO: 127 |
| SEQ ID NO: 39 | SEQ ID NO: 44 | SEQ ID NO: 128 |
| SEQ ID NO: 39 | SEQ ID NO: 44 | SEQ ID NO: 129 |
| SEQ ID NO: 39 | SEQ ID NO: 44 | SEQ ID NO: 130 |
| SEQ ID NO: 39 | SEQ ID NO: 44 | SEQ ID NO: 131 |
| SEQ ID NO: 39 | SEQ ID NO: 44 | SEQ ID NO: 132 |
| SEQ ID NO: 39 | SEQ ID NO: 44 | SEQ ID NO: 133 |
| SEQ ID NO: 39 | SEQ ID NO: 44 | SEQ ID NO: 134 |
| SEQ ID NO: 39 | SEQ ID NO: 44 | SEQ ID NO: 135 |
| SEQ ID NO: 39 | SEQ ID NO: 44 | SEQ ID NO: 136 |
| SEQ ID NO: 39 | SEQ ID NO: 44 | SEQ ID NO: 137 |
| SEQ ID NO: 39 | SEQ ID NO: 44 | SEQ ID NO: 138 |
| SEQ ID NO: 39 | SEQ ID NO: 44 | SEQ ID NO: 139 |
| SEQ ID NO: 39 | SEQ ID NO: 44 | SEQ ID NO: 140 |
| SEQ ID NO: 39 | SEQ ID NO: 44 | SEQ ID NO: 141 |
| SEQ ID NO: 39 | SEQ ID NO: 44 | SEQ ID NO: 142 |
| SEQ ID NO: 39 | SEQ ID NO: 44 | SEQ ID NO: 143 |
| SEQ ID NO: 39 | SEQ ID NO: 44 | SEQ ID NO: 144 |
| SEQ ID NO: 39 | SEQ ID NO: 44 | SEQ ID NO: 145 |
| SEQ ID NO: 39 | SEQ ID NO: 44 | SEQ ID NO: 146 |
| SEQ ID NO: 39 | SEQ ID NO: 44 | SEQ ID NO: 147 |
| SEQ ID NO: 39 | SEQ ID NO: 44 | SEQ ID NO: 148 |
| SEQ ID NO: 39 | SEQ ID NO: 44 | SEQ ID NO: 149 |
| SEQ ID NO: 39 | SEQ ID NO: 44 | SEQ ID NO: 150 |
| SEQ ID NO: 39 | SEQ ID NO: 44 | SEQ ID NO: 151 |
| SEQ ID NO: 39 | SEQ ID NO: 44 | SEQ ID NO: 152 |
| SEQ ID NO: 39 | SEQ ID NO: 44 | SEQ ID NO: 153 |
| SEQ ID NO: 39 | SEQ ID NO: 44 | SEQ ID NO: 154 |
| SEQ ID NO: 39 | SEQ ID NO: 44 | SEQ ID NO: 155 |
| SEQ ID NO: 39 | SEQ ID NO: 44 | SEQ ID NO: 156 |
| SEQ ID NO: 39 | SEQ ID NO: 44 | SEQ ID NO: 157 |
| SEQ ID NO: 39 | SEQ ID NO: 44 | SEQ ID NO: 158 |
| SEQ ID NO: 39 | SEQ ID NO: 44 | SEQ ID NO: 159 |
| SEQ ID NO: 39 | SEQ ID NO: 44 | SEQ ID NO: 160 |
| SEQ ID NO: 39 | SEQ ID NO: 44 | SEQ ID NO: 161 |
| SEQ ID NO: 39 | SEQ ID NO: 44 | SEQ ID NO: 162 |
| SEQ ID NO: 39 | SEQ ID NO: 44 | SEQ ID NO: 163 |
| SEQ ID NO: 39 | SEQ ID NO: 44 | SEQ ID NO: 164 |
| SEQ ID NO: 39 | SEQ ID NO: 44 | SEQ ID NO: 165 |
| SEQ ID NO: 39 | SEQ ID NO: 44 | SEQ ID NO: 166 |
| SEQ ID NO: 39 | SEQ ID NO: 44 | SEQ ID NO: 167 |
| SEQ ID NO: 39 | SEQ ID NO: 44 | SEQ ID NO: 168 |
| SEQ ID NO: 39 | SEQ ID NO: 44 | SEQ ID NO: 169 |
| SEQ ID NO: 39 | SEQ ID NO: 44 | SEQ ID NO: 170 |
| SEQ ID NO: 39 | SEQ ID NO: 44 | SEQ ID NO: 171 |
| SEQ ID NO: 39 | SEQ ID NO: 44 | SEQ ID NO: 172 |
| SEQ ID NO: 39 | SEQ ID NO: 44 | SEQ ID NO: 173 |
| SEQ ID NO: 39 | SEQ ID NO: 44 | SEQ ID NO: 174 |
| SEQ ID NO: 39 | SEQ ID NO: 44 | SEQ ID NO: 175 |
| SEQ ID NO: 39 | SEQ ID NO: 44 | SEQ ID NO: 176 |
| SEQ ID NO: 39 | SEQ ID NO: 44 | SEQ ID NO: 177 |
| SEQ ID NO: 39 | SEQ ID NO: 44 | SEQ ID NO: 178 |
| SEQ ID NO: 39 | SEQ ID NO: 44 | SEQ ID NO: 179 |
| SEQ ID NO: 39 | SEQ ID NO: 44 | SEQ ID NO: 180 |
| SEQ ID NO: 39 | SEQ ID NO: 44 | SEQ ID NO: 181 |
| SEQ ID NO: 39 | SEQ ID NO: 44 | SEQ ID NO: 182 |
| SEQ ID NO: 39 | SEQ ID NO: 44 | SEQ ID NO: 183 |
| SEQ ID NO: 39 | SEQ ID NO: 44 | SEQ ID NO: 184 |
| SEQ ID NO: 39 | SEQ ID NO: 44 | SEQ ID NO: 185 |
| SEQ ID NO: 39 | SEQ ID NO: 44 | SEQ ID NO: 186 |
| SEQ ID NO: 39 | SEQ ID NO: 44 | SEQ ID NO: 187 |
| SEQ ID NO: 39 | SEQ ID NO: 44 | SEQ ID NO: 188 |
| SEQ ID NO: 39 | SEQ ID NO: 44 | SEQ ID NO: 189 |
| SEQ ID NO: 39 | SEQ ID NO: 44 | SEQ ID NO: 190 |
| SEQ ID NO: 39 | SEQ ID NO: 44 | SEQ ID NO: 191 |
| SEQ ID NO: 39 | SEQ ID NO: 44 | SEQ ID NO: 192 |
| SEQ ID NO: 39 | SEQ ID NO: 44 | SEQ ID NO: 193 |
| SEQ ID NO: 39 | SEQ ID NO: 44 | SEQ ID NO: 194 |
| SEQ ID NO: 39 | SEQ ID NO: 44 | SEQ ID NO: 195 |
| SEQ ID NO: 39 | SEQ ID NO: 44 | SEQ ID NO: 196 |
| SEQ ID NO: 39 | SEQ ID NO: 44 | SEQ ID NO: 197 |
| SEQ ID NO: 39 | SEQ ID NO: 44 | SEQ ID NO: 198 |
| SEQ ID NO: 39 | SEQ ID NO: 44 | SEQ ID NO: 199 |
| SEQ ID NO: 39 | SEQ ID NO: 44 | SEQ ID NO: 200 |
| SEQ ID NO: 39 | SEQ ID NO: 44 | SEQ ID NO: 201 |
| SEQ ID NO: 39 | SEQ ID NO: 44 | SEQ ID NO: 202 |
| SEQ ID NO: 39 | SEQ ID NO: 44 | SEQ ID NO: 203 |
| SEQ ID NO: 39 | SEQ ID NO: 44 | SEQ ID NO: 204 |
| SEQ ID NO: 39 | SEQ ID NO: 44 | SEQ ID NO: 205 |
| SEQ ID NO: 39 | SEQ ID NO: 44 | SEQ ID NO: 206 |
| SEQ ID NO: 39 | SEQ ID NO: 44 | SEQ ID NO: 207 |
| SEQ ID NO: 39 | SEQ ID NO: 44 | SEQ ID NO: 208 |
| SEQ ID NO: 39 | SEQ ID NO: 44 | SEQ ID NO: 209 |
| SEQ ID NO: 39 | SEQ ID NO: 44 | SEQ ID NO: 210 |
| SEQ ID NO: 39 | SEQ ID NO: 44 | SEQ ID NO: 211 |
| SEQ ID NO: 39 | SEQ ID NO: 44 | SEQ ID NO: 212 |
| SEQ ID NO: 39 | SEQ ID NO: 44 | SEQ ID NO: 213 |
| SEQ ID NO: 39 | SEQ ID NO: 44 | SEQ ID NO: 214 |
| SEQ ID NO: 39 | SEQ ID NO: 44 | SEQ ID NO: 215 |
| SEQ ID NO: 39 | SEQ ID NO: 44 | SEQ ID NO: 216 |
| SEQ ID NO: 39 | SEQ ID NO: 44 | SEQ ID NO: 217 |
| SEQ ID NO: 39 | SEQ ID NO: 44 | SEQ ID NO: 218 |
| SEQ ID NO: 39 | SEQ ID NO: 44 | SEQ ID NO: 219 |
| SEQ ID NO: 39 | SEQ ID NO: 44 | SEQ ID NO: 220 |
| SEQ ID NO: 39 | SEQ ID NO: 44 | SEQ ID NO: 221 |
| SEQ ID NO: 41 | SEQ ID NO: 44 | SEQ ID NO: 120 |
| SEQ ID NO: 41 | SEQ ID NO: 44 | SEQ ID NO: 121 |
| SEQ ID NO: 41 | SEQ ID NO: 44 | SEQ ID NO: 122 |
| SEQ ID NO: 41 | SEQ ID NO: 44 | SEQ ID NO: 123 |
| SEQ ID NO: 41 | SEQ ID NO: 44 | SEQ ID NO: 124 |
| SEQ ID NO: 41 | SEQ ID NO: 44 | SEQ ID NO: 125 |
| SEQ ID NO: 41 | SEQ ID NO: 44 | SEQ ID NO: 126 |
| SEQ ID NO: 41 | SEQ ID NO: 44 | SEQ ID NO: 127 |
| SEQ ID NO: 41 | SEQ ID NO: 44 | SEQ ID NO: 128 |
| SEQ ID NO: 41 | SEQ ID NO: 44 | SEQ ID NO: 129 |
| SEQ ID NO: 41 | SEQ ID NO: 44 | SEQ ID NO: 130 |
| SEQ ID NO: 41 | SEQ ID NO: 44 | SEQ ID NO: 131 |
| SEQ ID NO: 41 | SEQ ID NO: 44 | SEQ ID NO: 132 |
| SEQ ID NO: 41 | SEQ ID NO: 44 | SEQ ID NO: 133 |
| SEQ ID NO: 41 | SEQ ID NO: 44 | SEQ ID NO: 134 |
| SEQ ID NO: 41 | SEQ ID NO: 44 | SEQ ID NO: 135 |
| SEQ ID NO: 41 | SEQ ID NO: 44 | SEQ ID NO: 136 |
| SEQ ID NO: 41 | SEQ ID NO: 44 | SEQ ID NO: 137 |
| SEQ ID NO: 41 | SEQ ID NO: 44 | SEQ ID NO: 138 |
| SEQ ID NO: 41 | SEQ ID NO: 44 | SEQ ID NO: 139 |
| SEQ ID NO: 41 | SEQ ID NO: 44 | SEQ ID NO: 140 |
| SEQ ID NO: 41 | SEQ ID NO: 44 | SEQ ID NO: 141 |
| SEQ ID NO: 41 | SEQ ID NO: 44 | SEQ ID NO: 142 |
| SEQ ID NO: 41 | SEQ ID NO: 44 | SEQ ID NO: 143 |
| SEQ ID NO: 41 | SEQ ID NO: 44 | SEQ ID NO: 144 |
| SEQ ID NO: 41 | SEQ ID NO: 44 | SEQ ID NO: 145 |
| SEQ ID NO: 41 | SEQ ID NO: 44 | SEQ ID NO: 146 |
| SEQ ID NO: 41 | SEQ ID NO: 44 | SEQ ID NO: 147 |
| SEQ ID NO: 41 | SEQ ID NO: 44 | SEQ ID NO: 148 |
| SEQ ID NO: 41 | SEQ ID NO: 44 | SEQ ID NO: 149 |
| SEQ ID NO: 41 | SEQ ID NO: 44 | SEQ ID NO: 150 |
| SEQ ID NO: 41 | SEQ ID NO: 44 | SEQ ID NO: 151 |
| SEQ ID NO: 41 | SEQ ID NO: 44 | SEQ ID NO: 152 |
| SEQ ID NO: 41 | SEQ ID NO: 44 | SEQ ID NO: 153 |
| SEQ ID NO: 41 | SEQ ID NO: 44 | SEQ ID NO: 154 |
| SEQ ID NO: 41 | SEQ ID NO: 44 | SEQ ID NO: 155 |
| SEQ ID NO: 41 | SEQ ID NO: 44 | SEQ ID NO: 156 |
| SEQ ID NO: 41 | SEQ ID NO: 44 | SEQ ID NO: 157 |
| SEQ ID NO: 41 | SEQ ID NO: 44 | SEQ ID NO: 158 |
| SEQ ID NO: 41 | SEQ ID NO: 44 | SEQ ID NO: 159 |
| SEQ ID NO: 41 | SEQ ID NO: 44 | SEQ ID NO: 160 |
| SEQ ID NO: 41 | SEQ ID NO: 44 | SEQ ID NO: 161 |
| SEQ ID NO: 41 | SEQ ID NO: 44 | SEQ ID NO: 162 |
| SEQ ID NO: 41 | SEQ ID NO: 44 | SEQ ID NO: 163 |
| SEQ ID NO: 41 | SEQ ID NO: 44 | SEQ ID NO: 164 |
| SEQ ID NO: 41 | SEQ ID NO: 44 | SEQ ID NO: 165 |
| SEQ ID NO: 41 | SEQ ID NO: 44 | SEQ ID NO: 166 |
| SEQ ID NO: 41 | SEQ ID NO: 44 | SEQ ID NO: 167 |
| SEQ ID NO: 41 | SEQ ID NO: 44 | SEQ ID NO: 168 |
| SEQ ID NO: 41 | SEQ ID NO: 44 | SEQ ID NO: 169 |
| SEQ ID NO: 41 | SEQ ID NO: 44 | SEQ ID NO: 170 |
| SEQ ID NO: 41 | SEQ ID NO: 44 | SEQ ID NO: 171 |
| SEQ ID NO: 41 | SEQ ID NO: 44 | SEQ ID NO: 172 |
| SEQ ID NO: 41 | SEQ ID NO: 44 | SEQ ID NO: 173 |
| SEQ ID NO: 41 | SEQ ID NO: 44 | SEQ ID NO: 174 |
| SEQ ID NO: 41 | SEQ ID NO: 44 | SEQ ID NO: 175 |
| SEQ ID NO: 41 | SEQ ID NO: 44 | SEQ ID NO: 176 |
| SEQ ID NO: 41 | SEQ ID NO: 44 | SEQ ID NO: 177 |
| SEQ ID NO: 41 | SEQ ID NO: 44 | SEQ ID NO: 178 |
| SEQ ID NO: 41 | SEQ ID NO: 44 | SEQ ID NO: 179 |
| SEQ ID NO: 41 | SEQ ID NO: 44 | SEQ ID NO: 180 |
| SEQ ID NO: 41 | SEQ ID NO: 44 | SEQ ID NO: 181 |
| SEQ ID NO: 41 | SEQ ID NO: 44 | SEQ ID NO: 182 |
| SEQ ID NO: 41 | SEQ ID NO: 44 | SEQ ID NO: 183 |
| SEQ ID NO: 41 | SEQ ID NO: 44 | SEQ ID NO: 184 |
| SEQ ID NO: 41 | SEQ ID NO: 44 | SEQ ID NO: 185 |
| SEQ ID NO: 41 | SEQ ID NO: 44 | SEQ ID NO: 186 |
| SEQ ID NO: 41 | SEQ ID NO: 44 | SEQ ID NO: 187 |
| SEQ ID NO: 41 | SEQ ID NO: 44 | SEQ ID NO: 188 |
| SEQ ID NO: 41 | SEQ ID NO: 44 | SEQ ID NO: 189 |
| SEQ ID NO: 41 | SEQ ID NO: 44 | SEQ ID NO: 190 |
| SEQ ID NO: 41 | SEQ ID NO: 44 | SEQ ID NO: 191 |
| SEQ ID NO: 41 | SEQ ID NO: 44 | SEQ ID NO: 192 |
| SEQ ID NO: 41 | SEQ ID NO: 44 | SEQ ID NO: 193 |
| SEQ ID NO: 41 | SEQ ID NO: 44 | SEQ ID NO: 194 |
| SEQ ID NO: 41 | SEQ ID NO: 44 | SEQ ID NO: 195 |
| SEQ ID NO: 41 | SEQ ID NO: 44 | SEQ ID NO: 196 |
| SEQ ID NO: 41 | SEQ ID NO: 44 | SEQ ID NO: 197 |
| SEQ ID NO: 41 | SEQ ID NO: 44 | SEQ ID NO: 198 |
| SEQ ID NO: 41 | SEQ ID NO: 44 | SEQ ID NO: 199 |
| SEQ ID NO: 41 | SEQ ID NO: 44 | SEQ ID NO: 200 |
| SEQ ID NO: 41 | SEQ ID NO: 44 | SEQ ID NO: 201 |
| SEQ ID NO: 41 | SEQ ID NO: 44 | SEQ ID NO: 202 |
| SEQ ID NO: 41 | SEQ ID NO: 44 | SEQ ID NO: 203 |
| SEQ ID NO: 41 | SEQ ID NO: 44 | SEQ ID NO: 204 |
| SEQ ID NO: 41 | SEQ ID NO: 44 | SEQ ID NO: 205 |
| SEQ ID NO: 41 | SEQ ID NO: 44 | SEQ ID NO: 206 |
| SEQ ID NO: 41 | SEQ ID NO: 44 | SEQ ID NO: 207 |
| SEQ ID NO: 41 | SEQ ID NO: 44 | SEQ ID NO: 208 |
| SEQ ID NO: 41 | SEQ ID NO: 44 | SEQ ID NO: 209 |
| SEQ ID NO: 41 | SEQ ID NO: 44 | SEQ ID NO: 210 |
| SEQ ID NO: 41 | SEQ ID NO: 44 | SEQ ID NO: 211 |
| SEQ ID NO: 41 | SEQ ID NO: 44 | SEQ ID NO: 212 |
| SEQ ID NO: 41 | SEQ ID NO: 44 | SEQ ID NO: 213 |
| SEQ ID NO: 41 | SEQ ID NO: 44 | SEQ ID NO: 214 |
| SEQ ID NO: 41 | SEQ ID NO: 44 | SEQ ID NO: 215 |
| SEQ ID NO: 41 | SEQ ID NO: 44 | SEQ ID NO: 216 |
| SEQ ID NO: 41 | SEQ ID NO: 44 | SEQ ID NO: 217 |
| SEQ ID NO: 41 | SEQ ID NO: 44 | SEQ ID NO: 218 |
| SEQ ID NO: 41 | SEQ ID NO: 44 | SEQ ID NO: 219 |
| SEQ ID NO: 41 | SEQ ID NO: 44 | SEQ ID NO: 220 |
| SEQ ID NO: 41 | SEQ ID NO: 44 | SEQ ID NO: 221 |
| SEQ ID NO: 43 | SEQ ID NO: 44 | SEQ ID NO: 120 |
| SEQ ID NO: 43 | SEQ ID NO: 44 | SEQ ID NO: 121 |
| SEQ ID NO: 43 | SEQ ID NO: 44 | SEQ ID NO: 122 |
| SEQ ID NO: 43 | SEQ ID NO: 44 | SEQ ID NO: 123 |
| SEQ ID NO: 43 | SEQ ID NO: 44 | SEQ ID NO: 124 |
| SEQ ID NO: 43 | SEQ ID NO: 44 | SEQ ID NO: 125 |
| SEQ ID NO: 43 | SEQ ID NO: 44 | SEQ ID NO: 126 |
| SEQ ID NO: 43 | SEQ ID NO: 44 | SEQ ID NO: 127 |
| SEQ ID NO: 43 | SEQ ID NO: 44 | SEQ ID NO: 128 |
| SEQ ID NO: 43 | SEQ ID NO: 44 | SEQ ID NO: 129 |
| SEQ ID NO: 43 | SEQ ID NO: 44 | SEQ ID NO: 130 |
| SEQ ID NO: 43 | SEQ ID NO: 44 | SEQ ID NO: 131 |
| SEQ ID NO: 43 | SEQ ID NO: 44 | SEQ ID NO: 132 |
| SEQ ID NO: 43 | SEQ ID NO: 44 | SEQ ID NO: 133 |
| SEQ ID NO: 43 | SEQ ID NO: 44 | SEQ ID NO: 134 |
| SEQ ID NO: 43 | SEQ ID NO: 44 | SEQ ID NO: 135 |
| SEQ ID NO: 43 | SEQ ID NO: 44 | SEQ ID NO: 136 |
| SEQ ID NO: 43 | SEQ ID NO: 44 | SEQ ID NO: 137 |
| SEQ ID NO: 43 | SEQ ID NO: 44 | SEQ ID NO: 138 |
| SEQ ID NO: 43 | SEQ ID NO: 44 | SEQ ID NO: 139 |
| SEQ ID NO: 43 | SEQ ID NO: 44 | SEQ ID NO: 140 |
| SEQ ID NO: 43 | SEQ ID NO: 44 | SEQ ID NO: 141 |
| SEQ ID NO: 43 | SEQ ID NO: 44 | SEQ ID NO: 142 |
| SEQ ID NO: 43 | SEQ ID NO: 44 | SEQ ID NO: 143 |
| SEQ ID NO: 43 | SEQ ID NO: 44 | SEQ ID NO: 144 |
| SEQ ID NO: 43 | SEQ ID NO: 44 | SEQ ID NO: 145 |
| SEQ ID NO: 43 | SEQ ID NO: 44 | SEQ ID NO: 146 |
| SEQ ID NO: 43 | SEQ ID NO: 44 | SEQ ID NO: 147 |
| SEQ ID NO: 43 | SEQ ID NO: 44 | SEQ ID NO: 148 |
| SEQ ID NO: 43 | SEQ ID NO: 44 | SEQ ID NO: 149 |
| SEQ ID NO: 43 | SEQ ID NO: 44 | SEQ ID NO: 150 |
| SEQ ID NO: 43 | SEQ ID NO: 44 | SEQ ID NO: 151 |
| SEQ ID NO: 43 | SEQ ID NO: 44 | SEQ ID NO: 152 |
| SEQ ID NO: 43 | SEQ ID NO: 44 | SEQ ID NO: 153 |
| SEQ ID NO: 43 | SEQ ID NO: 44 | SEQ ID NO: 154 |
| SEQ ID NO: 43 | SEQ ID NO: 44 | SEQ ID NO: 155 |
| SEQ ID NO: 43 | SEQ ID NO: 44 | SEQ ID NO: 156 |
| SEQ ID NO: 43 | SEQ ID NO: 44 | SEQ ID NO: 157 |
| SEQ ID NO: 43 | SEQ ID NO: 44 | SEQ ID NO: 158 |
| SEQ ID NO: 43 | SEQ ID NO: 44 | SEQ ID NO: 159 |
| SEQ ID NO: 43 | SEQ ID NO: 44 | SEQ ID NO: 160 |
| SEQ ID NO: 43 | SEQ ID NO: 44 | SEQ ID NO: 161 |
| SEQ ID NO: 43 | SEQ ID NO: 44 | SEQ ID NO: 162 |
| SEQ ID NO: 43 | SEQ ID NO: 44 | SEQ ID NO: 163 |
| SEQ ID NO: 43 | SEQ ID NO: 44 | SEQ ID NO: 164 |
| SEQ ID NO: 43 | SEQ ID NO: 44 | SEQ ID NO: 165 |
| SEQ ID NO: 43 | SEQ ID NO: 44 | SEQ ID NO: 166 |
| SEQ ID NO: 43 | SEQ ID NO: 44 | SEQ ID NO: 167 |
| SEQ ID NO: 43 | SEQ ID NO: 44 | SEQ ID NO: 168 |
| SEQ ID NO: 43 | SEQ ID NO: 44 | SEQ ID NO: 169 |
| SEQ ID NO: 43 | SEQ ID NO: 44 | SEQ ID NO: 170 |
| SEQ ID NO: 43 | SEQ ID NO: 44 | SEQ ID NO: 171 |
| SEQ ID NO: 43 | SEQ ID NO: 44 | SEQ ID NO: 172 |
| SEQ ID NO: 43 | SEQ ID NO: 44 | SEQ ID NO: 173 |
| SEQ ID NO: 43 | SEQ ID NO: 44 | SEQ ID NO: 174 |
| SEQ ID NO: 43 | SEQ ID NO: 44 | SEQ ID NO: 175 |
| SEQ ID NO: 43 | SEQ ID NO: 44 | SEQ ID NO: 176 |
| SEQ ID NO: 43 | SEQ ID NO: 44 | SEQ ID NO: 177 |
| SEQ ID NO: 43 | SEQ ID NO: 44 | SEQ ID NO: 178 |
| SEQ ID NO: 43 | SEQ ID NO: 44 | SEQ ID NO: 179 |
| SEQ ID NO: 43 | SEQ ID NO: 44 | SEQ ID NO: 180 |
| SEQ ID NO: 43 | SEQ ID NO: 44 | SEQ ID NO: 181 |
| SEQ ID NO: 43 | SEQ ID NO: 44 | SEQ ID NO: 182 |
| SEQ ID NO: 43 | SEQ ID NO: 44 | SEQ ID NO: 183 |
| SEQ ID NO: 43 | SEQ ID NO: 44 | SEQ ID NO: 184 |
| SEQ ID NO: 43 | SEQ ID NO: 44 | SEQ ID NO: 185 |
| SEQ ID NO: 43 | SEQ ID NO: 44 | SEQ ID NO: 186 |
| SEQ ID NO: 43 | SEQ ID NO: 44 | SEQ ID NO: 187 |
| SEQ ID NO: 43 | SEQ ID NO: 44 | SEQ ID NO: 188 |
| SEQ ID NO: 43 | SEQ ID NO: 44 | SEQ ID NO: 189 |
| SEQ ID NO: 43 | SEQ ID NO: 44 | SEQ ID NO: 190 |
| SEQ ID NO: 43 | SEQ ID NO: 44 | SEQ ID NO: 191 |
| SEQ ID NO: 43 | SEQ ID NO: 44 | SEQ ID NO: 192 |
| SEQ ID NO: 43 | SEQ ID NO: 44 | SEQ ID NO: 193 |
| SEQ ID NO: 43 | SEQ ID NO: 44 | SEQ ID NO: 194 |
| SEQ ID NO: 43 | SEQ ID NO: 44 | SEQ ID NO: 195 |
| SEQ ID NO: 43 | SEQ ID NO: 44 | SEQ ID NO: 196 |
| SEQ ID NO: 43 | SEQ ID NO: 44 | SEQ ID NO: 197 |
| SEQ ID NO: 43 | SEQ ID NO: 44 | SEQ ID NO: 198 |
| SEQ ID NO: 43 | SEQ ID NO: 44 | SEQ ID NO: 199 |
| SEQ ID NO: 43 | SEQ ID NO: 44 | SEQ ID NO: 200 |
| SEQ ID NO: 43 | SEQ ID NO: 44 | SEQ ID NO: 201 |
| SEQ ID NO: 43 | SEQ ID NO: 44 | SEQ ID NO: 202 |
| SEQ ID NO: 43 | SEQ ID NO: 44 | SEQ ID NO: 203 |
| SEQ ID NO: 43 | SEQ ID NO: 44 | SEQ ID NO: 204 |
| SEQ ID NO: 43 | SEQ ID NO: 44 | SEQ ID NO: 205 |
| SEQ ID NO: 43 | SEQ ID NO: 44 | SEQ ID NO: 206 |
| SEQ ID NO: 43 | SEQ ID NO: 44 | SEQ ID NO: 207 |
| SEQ ID NO: 43 | SEQ ID NO: 44 | SEQ ID NO: 208 |
| SEQ ID NO: 43 | SEQ ID NO: 44 | SEQ ID NO: 209 |
| SEQ ID NO: 43 | SEQ ID NO: 44 | SEQ ID NO: 210 |
| SEQ ID NO: 43 | SEQ ID NO: 44 | SEQ ID NO: 211 |
| SEQ ID NO: 43 | SEQ ID NO: 44 | SEQ ID NO: 212 |
| SEQ ID NO: 43 | SEQ ID NO: 44 | SEQ ID NO: 213 |
| SEQ ID NO: 43 | SEQ ID NO: 44 | SEQ ID NO: 214 |
| SEQ ID NO: 43 | SEQ ID NO: 44 | SEQ ID NO: 215 |
| SEQ ID NO: 43 | SEQ ID NO: 44 | SEQ ID NO: 216 |
| SEQ ID NO: 43 | SEQ ID NO: 44 | SEQ ID NO: 217 |
| SEQ ID NO: 43 | SEQ ID NO: 44 | SEQ ID NO: 218 |
| SEQ ID NO: 43 | SEQ ID NO: 44 | SEQ ID NO: 219 |
| SEQ ID NO: 43 | SEQ ID NO: 44 | SEQ ID NO: 220 |
| SEQ ID NO: 43 | SEQ ID NO: 44 | SEQ ID NO: 221 |
| SEQ ID NO: 43 | SEQ ID NO: 44 | SEQ ID NO: 329 |
| SEQ ID NO: 43 | SEQ ID NO: 44 | SEQ ID NO: 332 |
| SEQ ID NO: 43 | SEQ ID NO: 44 | SEQ ID NO: 335 |

TABLE 7
Example configurations of fusion proteins
Components (from an N-terminus to C-terminus direction)

| Heterologous | | Mutant Soluble |
|---|---|---|
| Protein | Linker(s) | ActRIIB-ECD polypeptide |
| SEQ ID NO: 39 | SEQ ID NO: 118 | SEQ ID NO: 119 |
| SEQ ID NO: 39 | SEQ ID NO: 118 | SEQ ID NO: 120 |
| SEQ ID NO: 39 | SEQ ID NO: 118 | SEQ ID NO: 121 |
| SEQ ID NO: 39 | SEQ ID NO: 118 | SEQ ID NO: 122 |
| SEQ ID NO: 39 | SEQ ID NO: 118 | SEQ ID NO: 123 |
| SEQ ID NO: 39 | SEQ ID NO: 118 | SEQ ID NO: 124 |
| SEQ ID NO: 39 | SEQ ID NO: 118 | SEQ ID NO: 125 |
| SEQ ID NO: 39 | SEQ ID NO: 118 | SEQ ID NO: 126 |
| SEQ ID NO: 39 | SEQ ID NO: 118 | SEQ ID NO: 127 |
| SEQ ID NO: 39 | SEQ ID NO: 118 | SEQ ID NO: 128 |
| SEQ ID NO: 39 | SEQ ID NO: 118 | SEQ ID NO: 129 |
| SEQ ID NO: 39 | SEQ ID NO: 118 | SEQ ID NO: 130 |
| SEQ ID NO: 39 | SEQ ID NO: 118 | SEQ ID NO: 131 |
| SEQ ID NO: 39 | SEQ ID NO: 118 | SEQ ID NO: 132 |
| SEQ ID NO: 39 | SEQ ID NO: 118 | SEQ ID NO: 133 |
| SEQ ID NO: 39 | SEQ ID NO: 118 | SEQ ID NO: 134 |
| SEQ ID NO: 39 | SEQ ID NO: 118 | SEQ ID NO: 135 |
| SEQ ID NO: 39 | SEQ ID NO: 118 | SEQ ID NO: 136 |
| SEQ ID NO: 39 | SEQ ID NO: 118 | SEQ ID NO: 137 |
| SEQ ID NO: 39 | SEQ ID NO: 118 | SEQ ID NO: 138 |
| SEQ ID NO: 39 | SEQ ID NO: 118 | SEQ ID NO: 139 |
| SEQ ID NO: 39 | SEQ ID NO: 118 | SEQ ID NO: 140 |
| SEQ ID NO: 39 | SEQ ID NO: 118 | SEQ ID NO: 141 |
| SEQ ID NO: 39 | SEQ ID NO: 118 | SEQ ID NO: 142 |
| SEQ ID NO: 39 | SEQ ID NO: 118 | SEQ ID NO: 143 |
| SEQ ID NO: 39 | SEQ ID NO: 118 | SEQ ID NO: 144 |
| SEQ ID NO: 39 | SEQ ID NO: 118 | SEQ ID NO: 145 |
| SEQ ID NO: 39 | SEQ ID NO: 118 | SEQ ID NO: 146 |
| SEQ ID NO: 39 | SEQ ID NO: 118 | SEQ ID NO: 147 |
| SEQ ID NO: 39 | SEQ ID NO: 118 | SEQ ID NO: 148 |
| SEQ ID NO: 39 | SEQ ID NO: 118 | SEQ ID NO: 149 |
| SEQ ID NO: 39 | SEQ ID NO: 118 | SEQ ID NO: 150 |
| SEQ ID NO: 39 | SEQ ID NO: 118 | SEQ ID NO: 151 |
| SEQ ID NO: 39 | SEQ ID NO: 118 | SEQ ID NO: 152 |
| SEQ ID NO: 39 | SEQ ID NO: 118 | SEQ ID NO: 153 |
| SEQ ID NO: 39 | SEQ ID NO: 118 | SEQ ID NO: 154 |
| SEQ ID NO: 39 | SEQ ID NO: 118 | SEQ ID NO: 155 |
| SEQ ID NO: 39 | SEQ ID NO: 118 | SEQ ID NO: 156 |
| SEQ ID NO: 39 | SEQ ID NO: 118 | SEQ ID NO: 157 |
| SEQ ID NO: 39 | SEQ ID NO: 118 | SEQ ID NO: 158 |
| SEQ ID NO: 39 | SEQ ID NO: 118 | SEQ ID NO: 159 |
| SEQ ID NO: 39 | SEQ ID NO: 118 | SEQ ID NO: 160 |
| SEQ ID NO: 39 | SEQ ID NO: 118 | SEQ ID NO: 161 |
| SEQ ID NO: 39 | SEQ ID NO: 118 | SEQ ID NO: 162 |
| SEQ ID NO: 39 | SEQ ID NO: 118 | SEQ ID NO: 163 |
| SEQ ID NO: 39 | SEQ ID NO: 118 | SEQ ID NO: 164 |
| SEQ ID NO: 39 | SEQ ID NO: 118 | SEQ ID NO: 165 |
| SEQ ID NO: 39 | SEQ ID NO: 118 | SEQ ID NO: 166 |
| SEQ ID NO: 39 | SEQ ID NO: 118 | SEQ ID NO: 167 |
| SEQ ID NO: 39 | SEQ ID NO: 118 | SEQ ID NO: 168 |
| SEQ ID NO: 39 | SEQ ID NO: 118 | SEQ ID NO: 169 |
| SEQ ID NO: 39 | SEQ ID NO: 118 | SEQ ID NO: 170 |
| SEQ ID NO: 39 | SEQ ID NO: 118 | SEQ ID NO: 171 |
| SEQ ID NO: 39 | SEQ ID NO: 118 | SEQ ID NO: 172 |
| SEQ ID NO: 39 | SEQ ID NO: 118 | SEQ ID NO: 173 |
| SEQ ID NO: 39 | SEQ ID NO: 118 | SEQ ID NO: 174 |
| SEQ ID NO: 39 | SEQ ID NO: 118 | SEQ ID NO: 175 |
| SEQ ID NO: 39 | SEQ ID NO: 118 | SEQ ID NO: 176 |
| SEQ ID NO: 39 | SEQ ID NO: 118 | SEQ ID NO: 177 |
| SEQ ID NO: 39 | SEQ ID NO: 118 | SEQ ID NO: 178 |
| SEQ ID NO: 39 | SEQ ID NO: 118 | SEQ ID NO: 179 |
| SEQ ID NO: 39 | SEQ ID NO: 118 | SEQ ID NO: 180 |
| SEQ ID NO: 39 | SEQ ID NO: 118 | SEQ ID NO: 181 |
| SEQ ID NO: 39 | SEQ ID NO: 118 | SEQ ID NO: 182 |
| SEQ ID NO: 39 | SEQ ID NO: 118 | SEQ ID NO: 183 |
| SEQ ID NO: 39 | SEQ ID NO: 118 | SEQ ID NO: 184 |
| SEQ ID NO: 39 | SEQ ID NO: 118 | SEQ ID NO: 185 |
| SEQ ID NO: 39 | SEQ ID NO: 118 | SEQ ID NO: 186 |
| SEQ ID NO: 39 | SEQ ID NO: 118 | SEQ ID NO: 187 |
| SEQ ID NO: 39 | SEQ ID NO: 118 | SEQ ID NO: 188 |
| SEQ ID NO: 39 | SEQ ID NO: 118 | SEQ ID NO: 189 |
| SEQ ID NO: 39 | SEQ ID NO: 118 | SEQ ID NO: 190 |
| SEQ ID NO: 39 | SEQ ID NO: 118 | SEQ ID NO: 191 |
| SEQ ID NO: 39 | SEQ ID NO: 118 | SEQ ID NO: 192 |
| SEQ ID NO: 39 | SEQ ID NO: 118 | SEQ ID NO: 193 |
| SEQ ID NO: 39 | SEQ ID NO: 118 | SEQ ID NO: 194 |
| SEQ ID NO: 39 | SEQ ID NO: 118 | SEQ ID NO: 195 |
| SEQ ID NO: 39 | SEQ ID NO: 118 | SEQ ID NO: 196 |
| SEQ ID NO: 39 | SEQ ID NO: 118 | SEQ ID NO: 197 |
| SEQ ID NO: 39 | SEQ ID NO: 118 | SEQ ID NO: 198 |
| SEQ ID NO: 39 | SEQ ID NO: 118 | SEQ ID NO: 199 |
| SEQ ID NO: 39 | SEQ ID NO: 118 | SEQ ID NO: 200 |
| SEQ ID NO: 39 | SEQ ID NO: 118 | SEQ ID NO: 201 |
| SEQ ID NO: 39 | SEQ ID NO: 118 | SEQ ID NO: 202 |
| SEQ ID NO: 39 | SEQ ID NO: 118 | SEQ ID NO: 203 |
| SEQ ID NO: 39 | SEQ ID NO: 118 | SEQ ID NO: 204 |
| SEQ ID NO: 39 | SEQ ID NO: 118 | SEQ ID NO: 205 |
| SEQ ID NO: 39 | SEQ ID NO: 118 | SEQ ID NO: 206 |
| SEQ ID NO: 39 | SEQ ID NO: 118 | SEQ ID NO: 207 |
| SEQ ID NO: 39 | SEQ ID NO: 118 | SEQ ID NO: 208 |
| SEQ ID NO: 39 | SEQ ID NO: 118 | SEQ ID NO: 209 |
| SEQ ID NO: 39 | SEQ ID NO: 118 | SEQ ID NO: 210 |
| SEQ ID NO: 39 | SEQ ID NO: 118 | SEQ ID NO: 211 |
| SEQ ID NO: 39 | SEQ ID NO: 118 | SEQ ID NO: 212 |
| SEQ ID NO: 39 | SEQ ID NO: 118 | SEQ ID NO: 213 |
| SEQ ID NO: 39 | SEQ ID NO: 118 | SEQ ID NO: 214 |
| SEQ ID NO: 39 | SEQ ID NO: 118 | SEQ ID NO: 215 |
| SEQ ID NO: 39 | SEQ ID NO: 118 | SEQ ID NO: 216 |
| SEQ ID NO: 39 | SEQ ID NO: 118 | SEQ ID NO: 217 |
| SEQ ID NO: 39 | SEQ ID NO: 118 | SEQ ID NO: 218 |
| SEQ ID NO: 39 | SEQ ID NO: 118 | SEQ ID NO: 219 |
| SEQ ID NO: 39 | SEQ ID NO: 118 | SEQ ID NO: 220 |
| SEQ ID NO: 39 | SEQ ID NO: 118 | SEQ ID NO: 221 |
| SEQ ID NO: 41 | SEQ ID NO: 118 | SEQ ID NO: 120 |
| SEQ ID NO: 41 | SEQ ID NO: 118 | SEQ ID NO: 121 |
| SEQ ID NO: 41 | SEQ ID NO: 118 | SEQ ID NO: 122 |
| SEQ ID NO: 41 | SEQ ID NO: 118 | SEQ ID NO: 123 |
| SEQ ID NO: 41 | SEQ ID NO: 118 | SEQ ID NO: 124 |
| SEQ ID NO: 41 | SEQ ID NO: 118 | SEQ ID NO: 125 |
| SEQ ID NO: 41 | SEQ ID NO: 118 | SEQ ID NO: 126 |
| SEQ ID NO: 41 | SEQ ID NO: 118 | SEQ ID NO: 127 |
| SEQ ID NO: 41 | SEQ ID NO: 118 | SEQ ID NO: 128 |
| SEQ ID NO: 41 | SEQ ID NO: 118 | SEQ ID NO: 129 |
| SEQ ID NO: 41 | SEQ ID NO: 118 | SEQ ID NO: 130 |
| SEQ ID NO: 41 | SEQ ID NO: 118 | SEQ ID NO: 131 |
| SEQ ID NO: 41 | SEQ ID NO: 118 | SEQ ID NO: 132 |
| SEQ ID NO: 41 | SEQ ID NO: 118 | SEQ ID NO: 133 |
| SEQ ID NO: 41 | SEQ ID NO: 118 | SEQ ID NO: 134 |
| SEQ ID NO: 41 | SEQ ID NO: 118 | SEQ ID NO: 135 |
| SEQ ID NO: 41 | SEQ ID NO: 118 | SEQ ID NO: 136 |
| SEQ ID NO: 41 | SEQ ID NO: 118 | SEQ ID NO: 137 |
| SEQ ID NO: 41 | SEQ ID NO: 118 | SEQ ID NO: 138 |
| SEQ ID NO: 41 | SEQ ID NO: 118 | SEQ ID NO: 139 |
| SEQ ID NO: 41 | SEQ ID NO: 118 | SEQ ID NO: 140 |
| SEQ ID NO: 41 | SEQ ID NO: 118 | SEQ ID NO: 141 |
| SEQ ID NO: 41 | SEQ ID NO: 118 | SEQ ID NO: 142 |
| SEQ ID NO: 41 | SEQ ID NO: 118 | SEQ ID NO: 143 |
| SEQ ID NO: 41 | SEQ ID NO: 118 | SEQ ID NO: 144 |
| SEQ ID NO: 41 | SEQ ID NO: 118 | SEQ ID NO: 145 |
| SEQ ID NO: 41 | SEQ ID NO: 118 | SEQ ID NO: 146 |
| SEQ ID NO: 41 | SEQ ID NO: 118 | SEQ ID NO: 147 |
| SEQ ID NO: 41 | SEQ ID NO: 118 | SEQ ID NO: 148 |
| SEQ ID NO: 41 | SEQ ID NO: 118 | SEQ ID NO: 149 |
| SEQ ID NO: 41 | SEQ ID NO: 118 | SEQ ID NO: 150 |
| SEQ ID NO: 41 | SEQ ID NO: 118 | SEQ ID NO: 151 |
| SEQ ID NO: 41 | SEQ ID NO: 118 | SEQ ID NO: 152 |
| SEQ ID NO: 41 | SEQ ID NO: 118 | SEQ ID NO: 153 |
| SEQ ID NO: 41 | SEQ ID NO: 118 | SEQ ID NO: 154 |
| SEQ ID NO: 41 | SEQ ID NO: 118 | SEQ ID NO: 155 |
| SEQ ID NO: 41 | SEQ ID NO: 118 | SEQ ID NO: 156 |
| SEQ ID NO: 41 | SEQ ID NO: 118 | SEQ ID NO: 157 |
| SEQ ID NO: 41 | SEQ ID NO: 118 | SEQ ID NO: 158 |
| SEQ ID NO: 41 | SEQ ID NO: 118 | SEQ ID NO: 159 |
| SEQ ID NO: 41 | SEQ ID NO: 118 | SEQ ID NO: 160 |
| SEQ ID NO: 41 | SEQ ID NO: 118 | SEQ ID NO: 161 |
| SEQ ID NO: 41 | SEQ ID NO: 118 | SEQ ID NO: 162 |
| SEQ ID NO: 41 | SEQ ID NO: 118 | SEQ ID NO: 163 |
| SEQ ID NO: 41 | SEQ ID NO: 118 | SEQ ID NO: 164 |
| SEQ ID NO: 41 | SEQ ID NO: 118 | SEQ ID NO: 165 |
| SEQ ID NO: 41 | SEQ ID NO: 118 | SEQ ID NO: 166 |
| SEQ ID NO: 41 | SEQ ID NO: 118 | SEQ ID NO: 167 |
| SEQ ID NO: 41 | SEQ ID NO: 118 | SEQ ID NO: 168 |
| SEQ ID NO: 41 | SEQ ID NO: 118 | SEQ ID NO: 169 |
| SEQ ID NO: 41 | SEQ ID NO: 118 | SEQ ID NO: 170 |
| SEQ ID NO: 41 | SEQ ID NO: 118 | SEQ ID NO: 171 |
| SEQ ID NO: 41 | SEQ ID NO: 118 | SEQ ID NO: 172 |
| SEQ ID NO: 41 | SEQ ID NO: 118 | SEQ ID NO: 173 |
| SEQ ID NO: 41 | SEQ ID NO: 118 | SEQ ID NO: 174 |
| SEQ ID NO: 41 | SEQ ID NO: 118 | SEQ ID NO: 175 |
| SEQ ID NO: 41 | SEQ ID NO: 118 | SEQ ID NO: 176 |
| SEQ ID NO: 41 | SEQ ID NO: 118 | SEQ ID NO: 177 |
| SEQ ID NO: 41 | SEQ ID NO: 118 | SEQ ID NO: 178 |
| SEQ ID NO: 41 | SEQ ID NO: 118 | SEQ ID NO: 179 |
| SEQ ID NO: 41 | SEQ ID NO: 118 | SEQ ID NO: 180 |
| SEQ ID NO: 41 | SEQ ID NO: 118 | SEQ ID NO: 181 |
| SEQ ID NO: 41 | SEQ ID NO: 118 | SEQ ID NO: 182 |
| SEQ ID NO: 41 | SEQ ID NO: 118 | SEQ ID NO: 183 |
| SEQ ID NO: 41 | SEQ ID NO: 118 | SEQ ID NO: 184 |
| SEQ ID NO: 41 | SEQ ID NO: 118 | SEQ ID NO: 185 |
| SEQ ID NO: 41 | SEQ ID NO: 118 | SEQ ID NO: 186 |
| SEQ ID NO: 41 | SEQ ID NO: 118 | SEQ ID NO: 187 |
| SEQ ID NO: 41 | SEQ ID NO: 118 | SEQ ID NO: 188 |
| SEQ ID NO: 41 | SEQ ID NO: 118 | SEQ ID NO: 189 |
| SEQ ID NO: 41 | SEQ ID NO: 118 | SEQ ID NO: 190 |
| SEQ ID NO: 41 | SEQ ID NO: 118 | SEQ ID NO: 191 |
| SEQ ID NO: 41 | SEQ ID NO: 118 | SEQ ID NO: 192 |
| SEQ ID NO: 41 | SEQ ID NO: 118 | SEQ ID NO: 193 |
| SEQ ID NO: 41 | SEQ ID NO: 118 | SEQ ID NO: 194 |
| SEQ ID NO: 41 | SEQ ID NO: 118 | SEQ ID NO: 195 |
| SEQ ID NO: 41 | SEQ ID NO: 118 | SEQ ID NO: 196 |
| SEQ ID NO: 41 | SEQ ID NO: 118 | SEQ ID NO: 197 |
| SEQ ID NO: 41 | SEQ ID NO: 118 | SEQ ID NO: 198 |
| SEQ ID NO: 41 | SEQ ID NO: 118 | SEQ ID NO: 199 |
| SEQ ID NO: 41 | SEQ ID NO: 118 | SEQ ID NO: 200 |
| SEQ ID NO: 41 | SEQ ID NO: 118 | SEQ ID NO: 201 |
| SEQ ID NO: 41 | SEQ ID NO: 118 | SEQ ID NO: 202 |
| SEQ ID NO: 41 | SEQ ID NO: 118 | SEQ ID NO: 203 |
| SEQ ID NO: 41 | SEQ ID NO: 118 | SEQ ID NO: 204 |
| SEQ ID NO: 41 | SEQ ID NO: 118 | SEQ ID NO: 205 |
| SEQ ID NO: 41 | SEQ ID NO: 118 | SEQ ID NO: 206 |
| SEQ ID NO: 41 | SEQ ID NO: 118 | SEQ ID NO: 207 |
| SEQ ID NO: 41 | SEQ ID NO: 118 | SEQ ID NO: 208 |
| SEQ ID NO: 41 | SEQ ID NO: 118 | SEQ ID NO: 209 |
| SEQ ID NO: 41 | SEQ ID NO: 118 | SEQ ID NO: 210 |
| SEQ ID NO: 41 | SEQ ID NO: 118 | SEQ ID NO: 211 |
| SEQ ID NO: 41 | SEQ ID NO: 118 | SEQ ID NO: 212 |
| SEQ ID NO: 41 | SEQ ID NO: 118 | SEQ ID NO: 213 |
| SEQ ID NO: 41 | SEQ ID NO: 118 | SEQ ID NO: 214 |
| SEQ ID NO: 41 | SEQ ID NO: 118 | SEQ ID NO: 215 |
| SEQ ID NO: 41 | SEQ ID NO: 118 | SEQ ID NO: 216 |
| SEQ ID NO: 41 | SEQ ID NO: 118 | SEQ ID NO: 217 |
| SEQ ID NO: 41 | SEQ ID NO: 118 | SEQ ID NO: 218 |
| SEQ ID NO: 41 | SEQ ID NO: 118 | SEQ ID NO: 219 |
| SEQ ID NO: 41 | SEQ ID NO: 118 | SEQ ID NO: 220 |
| SEQ ID NO: 41 | SEQ ID NO: 118 | SEQ ID NO: 221 |
| SEQ ID NO: 43 | SEQ ID NO: 118 | SEQ ID NO: 120 |
| SEQ ID NO: 43 | SEQ ID NO: 118 | SEQ ID NO: 121 |
| SEQ ID NO: 43 | SEQ ID NO: 118 | SEQ ID NO: 122 |
| SEQ ID NO: 43 | SEQ ID NO: 118 | SEQ ID NO: 123 |
| SEQ ID NO: 43 | SEQ ID NO: 118 | SEQ ID NO: 124 |
| SEQ ID NO: 43 | SEQ ID NO: 118 | SEQ ID NO: 125 |
| SEQ ID NO: 43 | SEQ ID NO: 118 | SEQ ID NO: 126 |
| SEQ ID NO: 43 | SEQ ID NO: 118 | SEQ ID NO: 127 |
| SEQ ID NO: 43 | SEQ ID NO: 118 | SEQ ID NO: 128 |
| SEQ ID NO: 43 | SEQ ID NO: 118 | SEQ ID NO: 129 |
| SEQ ID NO: 43 | SEQ ID NO: 118 | SEQ ID NO: 130 |
| SEQ ID NO: 43 | SEQ ID NO: 118 | SEQ ID NO: 131 |
| SEQ ID NO: 43 | SEQ ID NO: 118 | SEQ ID NO: 132 |
| SEQ ID NO: 43 | SEQ ID NO: 118 | SEQ ID NO: 133 |
| SEQ ID NO: 43 | SEQ ID NO: 118 | SEQ ID NO: 134 |
| SEQ ID NO: 43 | SEQ ID NO: 118 | SEQ ID NO: 135 |
| SEQ ID NO: 43 | SEQ ID NO: 118 | SEQ ID NO: 136 |
| SEQ ID NO: 43 | SEQ ID NO: 118 | SEQ ID NO: 137 |
| SEQ ID NO: 43 | SEQ ID NO: 118 | SEQ ID NO: 138 |
| SEQ ID NO: 43 | SEQ ID NO: 118 | SEQ ID NO: 139 |
| SEQ ID NO: 43 | SEQ ID NO: 118 | SEQ ID NO: 140 |
| SEQ ID NO: 43 | SEQ ID NO: 118 | SEQ ID NO: 141 |
| SEQ ID NO: 43 | SEQ ID NO: 118 | SEQ ID NO: 142 |
| SEQ ID NO: 43 | SEQ ID NO: 118 | SEQ ID NO: 143 |
| SEQ ID NO: 43 | SEQ ID NO: 118 | SEQ ID NO: 144 |
| SEQ ID NO: 43 | SEQ ID NO: 118 | SEQ ID NO: 145 |
| SEQ ID NO: 43 | SEQ ID NO: 118 | SEQ ID NO: 146 |
| SEQ ID NO: 43 | SEQ ID NO: 118 | SEQ ID NO: 147 |
| SEQ ID NO: 43 | SEQ ID NO: 118 | SEQ ID NO: 148 |
| SEQ ID NO: 43 | SEQ ID NO: 118 | SEQ ID NO: 149 |
| SEQ ID NO: 43 | SEQ ID NO: 118 | SEQ ID NO: 150 |
| SEQ ID NO: 43 | SEQ ID NO: 118 | SEQ ID NO: 151 |
| SEQ ID NO: 43 | SEQ ID NO: 118 | SEQ ID NO: 152 |
| SEQ ID NO: 43 | SEQ ID NO: 118 | SEQ ID NO: 153 |
| SEQ ID NO: 43 | SEQ ID NO: 118 | SEQ ID NO: 154 |
| SEQ ID NO: 43 | SEQ ID NO: 118 | SEQ ID NO: 155 |
| SEQ ID NO: 43 | SEQ ID NO: 118 | SEQ ID NO: 156 |
| SEQ ID NO: 43 | SEQ ID NO: 118 | SEQ ID NO: 157 |
| SEQ ID NO: 43 | SEQ ID NO: 118 | SEQ ID NO: 158 |
| SEQ ID NO: 43 | SEQ ID NO: 118 | SEQ ID NO: 159 |
| SEQ ID NO: 43 | SEQ ID NO: 118 | SEQ ID NO: 160 |
| SEQ ID NO: 43 | SEQ ID NO: 118 | SEQ ID NO: 161 |
| SEQ ID NO: 43 | SEQ ID NO: 118 | SEQ ID NO: 162 |
| SEQ ID NO: 43 | SEQ ID NO: 118 | SEQ ID NO: 163 |
| SEQ ID NO: 43 | SEQ ID NO: 118 | SEQ ID NO: 164 |
| SEQ ID NO: 43 | SEQ ID NO: 118 | SEQ ID NO: 165 |
| SEQ ID NO: 43 | SEQ ID NO: 118 | SEQ ID NO: 166 |
| SEQ ID NO: 43 | SEQ ID NO: 118 | SEQ ID NO: 167 |
| SEQ ID NO: 43 | SEQ ID NO: 118 | SEQ ID NO: 168 |
| SEQ ID NO: 43 | SEQ ID NO: 118 | SEQ ID NO: 169 |
| SEQ ID NO: 43 | SEQ ID NO: 118 | SEQ ID NO: 170 |
| SEQ ID NO: 43 | SEQ ID NO: 118 | SEQ ID NO: 171 |
| SEQ ID NO: 43 | SEQ ID NO: 118 | SEQ ID NO: 172 |
| SEQ ID NO: 43 | SEQ ID NO: 118 | SEQ ID NO: 173 |
| SEQ ID NO: 43 | SEQ ID NO: 118 | SEQ ID NO: 174 |
| SEQ ID NO: 43 | SEQ ID NO: 118 | SEQ ID NO: 175 |
| SEQ ID NO: 43 | SEQ ID NO: 118 | SEQ ID NO: 176 |
| SEQ ID NO: 43 | SEQ ID NO: 118 | SEQ ID NO: 177 |
| SEQ ID NO: 43 | SEQ ID NO: 118 | SEQ ID NO: 178 |
| SEQ ID NO: 43 | SEQ ID NO: 118 | SEQ ID NO: 179 |
| SEQ ID NO: 43 | SEQ ID NO: 118 | SEQ ID NO: 180 |
| SEQ ID NO: 43 | SEQ ID NO: 118 | SEQ ID NO: 181 |
| SEQ ID NO: 43 | SEQ ID NO: 118 | SEQ ID NO: 182 |
| SEQ ID NO: 43 | SEQ ID NO: 118 | SEQ ID NO: 183 |
| SEQ ID NO: 43 | SEQ ID NO: 118 | SEQ ID NO: 184 |
| SEQ ID NO: 43 | SEQ ID NO: 118 | SEQ ID NO: 185 |
| SEQ ID NO: 43 | SEQ ID NO: 118 | SEQ ID NO: 186 |
| SEQ ID NO: 43 | SEQ ID NO: 118 | SEQ ID NO: 187 |
| SEQ ID NO: 43 | SEQ ID NO: 118 | SEQ ID NO: 188 |
| SEQ ID NO: 43 | SEQ ID NO: 118 | SEQ ID NO: 189 |
| SEQ ID NO: 43 | SEQ ID NO: 118 | SEQ ID NO: 190 |
| SEQ ID NO: 43 | SEQ ID NO: 118 | SEQ ID NO: 191 |
| SEQ ID NO: 43 | SEQ ID NO: 118 | SEQ ID NO: 192 |
| SEQ ID NO: 43 | SEQ ID NO: 118 | SEQ ID NO: 193 |
| SEQ ID NO: 43 | SEQ ID NO: 118 | SEQ ID NO: 194 |
| SEQ ID NO: 43 | SEQ ID NO: 118 | SEQ ID NO: 195 |
| SEQ ID NO: 43 | SEQ ID NO: 118 | SEQ ID NO: 196 |
| SEQ ID NO: 43 | SEQ ID NO: 118 | SEQ ID NO: 197 |
| SEQ ID NO: 43 | SEQ ID NO: 118 | SEQ ID NO: 198 |
| SEQ ID NO: 43 | SEQ ID NO: 118 | SEQ ID NO: 199 |
| SEQ ID NO: 43 | SEQ ID NO: 118 | SEQ ID NO: 200 |
| SEQ ID NO: 43 | SEQ ID NO: 118 | SEQ ID NO: 201 |
| SEQ ID NO: 43 | SEQ ID NO: 118 | SEQ ID NO: 202 |
| SEQ ID NO: 43 | SEQ ID NO: 118 | SEQ ID NO: 203 |
| SEQ ID NO: 43 | SEQ ID NO: 118 | SEQ ID NO: 204 |
| SEQ ID NO: 43 | SEQ ID NO: 118 | SEQ ID NO: 205 |
| SEQ ID NO: 43 | SEQ ID NO: 118 | SEQ ID NO: 206 |
| SEQ ID NO: 43 | SEQ ID NO: 118 | SEQ ID NO: 207 |
| SEQ ID NO: 43 | SEQ ID NO: 118 | SEQ ID NO: 208 |
| SEQ ID NO: 43 | SEQ ID NO: 118 | SEQ ID NO: 209 |
| SEQ ID NO: 43 | SEQ ID NO: 118 | SEQ ID NO: 210 |
| SEQ ID NO: 43 | SEQ ID NO: 118 | SEQ ID NO: 211 |
| SEQ ID NO: 43 | SEQ ID NO: 118 | SEQ ID NO: 212 |
| SEQ ID NO: 43 | SEQ ID NO: 118 | SEQ ID NO: 213 |
| SEQ ID NO: 43 | SEQ ID NO: 118 | SEQ ID NO: 214 |
| SEQ ID NO: 43 | SEQ ID NO: 118 | SEQ ID NO: 215 |
| SEQ ID NO: 43 | SEQ ID NO: 118 | SEQ ID NO: 216 |
| SEQ ID NO: 43 | SEQ ID NO: 118 | SEQ ID NO: 217 |
| SEQ ID NO: 43 | SEQ ID NO: 118 | SEQ ID NO: 218 |
| SEQ ID NO: 43 | SEQ ID NO: 118 | SEQ ID NO: 219 |
| SEQ ID NO: 43 | SEQ ID NO: 118 | SEQ ID NO: 220 |
| SEQ ID NO: 43 | SEQ ID NO: 118 | SEQ ID NO: 221 |
| SEQ ID NO: 43 | SEQ ID NO: 118 | SEQ ID NO: 329 |
| SEQ ID NO: 43 | SEQ ID NO: 118 | SEQ ID NO: 332 |
| SEQ ID NO: 43 | SEQ ID NO: 118 | SEQ ID NO: 335 |

In some embodiments, the isolated protein comprising the mutant soluble ActRIIA-ECD or ActRIIB-ECD polypeptide and other components may comprise higher level of sialylation compared to otherwise identical isolated protein in which the glycosylation site on the mutant soluble ActRIIA-ECD or ActRIIB-ECD polypeptide is more removed. In some embodiments, provided herein is a sample comprising mutant soluble ActRIIA-ECD or ActRIIB-ECD polypeptides described herein, e.g., comprising a certain percentage of sialylated glycans. For example, a sample of isolated protein may comprise at least 5%, 10%, 20%, 30%, 40%, 50%, 60%, 70%, 80%, or 90% sialylated glycans. In some embodiments, a sample of the mutant soluble ActRIIA-ECD or ActRIIB-ECD comprises at least 5%, 10%, 20%, 30%, 40%, 50%, 60%, 70%, 80%, or 90% sialylated glycans at the position corresponding to N41 of SEQ ID NO: 1. In some examples, a sample of the isolated protein may comprise at least 40% sialylated glycans. The level of sialylation may be measured by any suitable method, including those described in Gerhild Zauner et al., Electrophoresis. 2011 December; 32 (24): 3456-66. doi: 10.1002/elps.201100247, which is incorporated by reference herein in its entirety.

### Polynucleotides

In another aspect, the present disclosure provides nucleic acid molecules comprising one or more polynucleotides encoding an isolated protein or component(s) thereof. In some examples, a nucleic acid molecule may comprise a single polynucleotide encoding the full-length isolated protein. In various embodiments, the nucleic acid molecules comprise the polynucleotides described herein, and further comprise a polynucleotide encoding at least one heterologous protein described herein. In various embodiments, the nucleic acid molecules further comprise polynucleotides encoding the linkers or hinge linkers described herein. In some embodiments, the polynucleotides encodes any one of the polypeptide sequences of the fusion protein of SEQ ID NO: 222. In some embodiments, the polynucleotides encodes any one of the polypeptide sequences of a fusion protein set forth in any rows of Tables 2-7.

The subject nucleic acids may be single-stranded or double stranded. Such nucleic acids may be DNA or RNA molecules. DNA includes, for example, cDNA, genomic DNA, synthetic DNA, DNA amplified by PCR, and combinations thereof. Genomic DNA encoding isolated protein is obtained from genomic libraries which are available for a number of species. Synthetic DNA is available from chemical synthesis of overlapping oligonucleotide fragments followed by assembly of the fragments to reconstitute part or all of the coding regions and flanking sequences. RNA may be obtained from prokaryotic expression vectors which direct high-level synthesis of mRNA, such as vectors using T7 promoters and RNA polymerase. cDNA may be obtained from libraries prepared from mRNA isolated from various tissues that express the isolated protein. The DNA molecules of the disclosure include full-length genes as well as polynucleotides and fragments thereof. The full-length gene may also include sequences encoding the N-terminal signal sequence.

Such nucleic acids may be used, for example, in methods for making the isolated protein. In various embodiments, the polynucleotides encodes any one of the polypeptide sequences set forth in SEQ ID NOs: 119, 223, 120-221, 224-325, 328, 329, 331, 332, 334, 335, or 222, or fusion proteins described in Tables 2-7. In various embodiments, the polynucleotides encode a polypeptide having an amino acid sequence at least 80%, 85%, 90%, 95%, 96%, 97%, 98% or 99% identity to any one of the polypeptides sequences set forth in SEQ ID NOs: 119, 223, 120-221, 224-325, or 222, or fusion proteins described in Tables 2-7. In various embodiments, the polynucleotides encode a polypeptide having at least 90% identity to any one of the polypeptides sequences set forth in SEQ ID NOs: SEQ ID NOs: 119, 223, 120-221, 224-325, 328, 329, 331, 332, 334, 335, or 222, or fusion proteins described in Tables 2-7. In various embodiments, the polynucleotides encode a polypeptide having an amino acid sequence at least 95% identity to any one of the polypeptides sequences set forth in SEQ ID NOs: SEQ ID NOs: 119, 223, 120-221, 224-325, 328, 329, 331, 332, 334, 335, or 222, or fusion proteins described in Tables 2-7.

In various embodiments, the nucleic acid sequences of the present disclosure can be isolated, recombinant, and/or fused with a heterologous nucleotide sequence, or in a DNA library.

In various embodiments, the present disclosure provides nucleic acid molecules which hybridize under stringent or moderate conditions with the polypeptide-encoding regions of the polynucleotides described herein. One of ordinary skill in the art will understand readily that appropriate stringency conditions, which promote DNA hybridization can be varied. For example, one could perform the hybridization at 6.0 X sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

In various embodiments, the recombinant nucleic acids of the present disclosure may be operably linked to one or more regulatory nucleotide sequences in an expression construct. Regulatory sequences are art-recognized and are selected to direct expression of the isolated protein. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the present disclosure. The promoters may be either naturally occurring promoters, or hybrid promoters that combine elements of more than one promoter. An expression construct may be present in a cell on an episome, such as a plasmid, or the expression construct may be inserted in a chromosome. In various embodiments, the expression vector contains a selectable marker gene to allow the selection of transformed host cells. Selectable marker genes are well known in the art and will vary with the host cell used.

In another aspect, the nucleic acid molecule may be provided in an expression vector comprising a nucleotide sequence encoding the isolated protein and operably linked to at least one regulatory sequence. An “expression vector” is a type of vector that can direct the expression of a chosen polynucleotide. The term “expression vector” refers to a plasmid, phage, virus or vector for expressing a polypeptide from a polynucleotide sequence. Vectors suitable for expression in host cells are readily available and the nucleic acid molecules are inserted into the vectors using standard recombinant DNA techniques. Such vectors can include a wide variety of expression control sequences that control the expression of a DNA sequence when operatively linked to it may be used in these vectors to express DNA sequences encoding the isolated protein. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., PhoS, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered. An exemplary expression vector suitable for expression of vActRIIB is the pDSRa, (described in WO 90/14363, herein incorporated by reference) and its derivatives, containing vActRIIB polynucleotides, as well as any additional suitable vectors known in the art or described below.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant isolated protein include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Suitable vectors also include some mammalian expression vectors, which contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

In various embodiments, a vector may be designed for production of the subject isolated protein in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject isolated protein in cells propagated in culture, e.g., to produce proteins, including fusion proteins or variant proteins, for purification.

This present disclosure also pertains to a host cell transfected with a recombinant gene including a nucleotide sequence coding an amino acid for one or more of the subject isolated protein. The host cell may be any prokaryotic or eukaryotic cell. For example, an isolated protein of the present disclosure may be expressed in bacterial cells such as E. coli, insect cells (e.g., using a baculovirus expression system), yeast, or mammalian cells. Other suitable host cells are known to those skilled in the art.

### Methods of Production

Accordingly, the present disclosure further pertains to methods of producing the subject isolated protein. In general, the methods may include culturing the host cell herein under conditions promoting the expression of the protein, and recovering the protein. The protein may be purified, e.g., using one or more of Protein A chromatography, size exclusion chromatography, and ion exchange chromatography.

For example, a host cell transfected with an expression vector encoding an isolated protein can be cultured under appropriate conditions to allow expression of the isolated protein to occur. The isolated protein may be secreted and isolated from a mixture of cells and medium containing the isolated protein. Alternatively, the isolated protein may be retained cytoplasmically or in a membrane fraction and the cells harvested, lysed and the protein isolated. A cell culture includes host cells, media and other byproducts. Suitable media for cell culture are well known in the art.

The polypeptides and proteins of the present disclosure can be purified according to protein purification techniques are well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The purified may be a composition, isolatable from other components, wherein the protein is purified to any degree relative to its naturally-obtainable state. A purified protein may be free from the environment in which it may naturally occur. Generally, “purified” may refer to a polypeptide composition that has been subjected to fractionation to remove various other components, and which composition substantially retains its expressed biological activity.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography; isoelectric focusing; gel electrophoresis; and combinations of these techniques. As is generally known in the art, it is believed that the order of conducting the various purification steps may be changed, or that certain steps may be omitted, and still result in a suitable method for the preparation of a substantially purified polypeptide.

An exemplary configuration of a synthetic DNA cassette encoding an isolated protein can be generally described as comprising the following elements: 1) a signal peptide (or leader sequence) placed at the N-terminus, which can be either the native signal peptide of ActRIIB (e.g., SEQ ID NO: 49) or any surrogate signal peptide capable of mediating the processing and secretion of secreted proteins (e.g., by using the human immunoglobulin light chain leader sequence (SEQ ID NO: 50) as a surrogate signal peptide, efficient secretion of mutant soluble ActRIIB in CHO cells can be achieved); 2) a mutant soluble ActRIIB-ECD polypeptide sequence (e.g., any one of SEQ ID NOs: 119-221) fused to the signal peptide sequence; 3) a peptide linker sequence (e.g., SEQ ID NO: 44) and hinge linker sequence (SEQ ID NO: 118), and 4) an Fc domain (e.g., SEQ ID NOs: 39, 41 or 43) fused to the mutant soluble ActRIIB-ECD polypeptide sequence by the peptide/hinge linker.

### Pharmaceutical Compositions

In another aspect, the present disclosure provides a pharmaceutical composition comprising the isolated protein herein in admixture with a pharmaceutically acceptable carrier. Such pharmaceutically acceptable carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

When parenteral administration is contemplated, the therapeutic pharmaceutical compositions may be in the form of a pyrogen-free, parenterally acceptable aqueous solution comprising the desired isolated protein in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions.

In various embodiments, the therapeutic pharmaceutical compositions may be formulated for targeted delivery using a colloidal dispersion system. Colloidal dispersion systems include macromolecule complexes, nanocapsules, microspheres, beads, and lipid-based systems including oil-in-water emulsions, micelles, mixed micelles, and liposomes. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

In various embodiments, oral administration of the pharmaceutical compositions is contemplated. Pharmaceutical compositions that are administered in this fashion can be formulated with or without those carriers customarily used in the compounding of solid dosage forms such as tablets and capsules. In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavoring, coloring, perfuming, and preservative agents.

In various embodiments, topical administration of the pharmaceutical compositions, either to skin or to mucosal membranes, is contemplated. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject composition of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Additional pharmaceutical compositions contemplated for use herein include formulations involving polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art.

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the polypeptide is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.1 mg/kg to up to about 100 mg/kg or more, depending on the factors mentioned above. Polypeptide compositions may be preferably injected or administered intravenously. Long-acting pharmaceutical compositions may be administered every three to four days, every week, or biweekly depending on the half-life and clearance rate of the particular formulation. The frequency of dosing will depend upon the pharmacokinetic parameters of the polypeptide in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, or intraperitoneal; as well as intranasal, enteral, topical, sublingual, urethral, vaginal, or rectal means, by sustained release systems or by implantation devices. In some examples, the pharmaceutical composition is formulated for administration by a route selected from the group consisting of: subcutaneous, intramuscular, intravenous, and intrathecal administration.

Where desired, the compositions may be administered by bolus injection or continuously by infusion, or by implantation device. Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In various embodiments, the present disclosure provides a method for treating muscle wasting in a subject, comprising administering to the subject a therapeutically amount of the isolated protein in admixture with a pharmaceutically acceptable carrier. For example, the isolated protein may comprise an amino acid sequence selected from the group consisting of SEQ ID NOs: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating bone disorders in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating metabolic disorders in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating fibrosis in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating autoimmune/inflammatory disease in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating cardiovascular disease in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating cancer in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating renal disease in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating arthritis in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating anorexia in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating liver disease in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method of inducing stem cell growth for tissue repair or organ regeneration in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating anemia in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating pain in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In various embodiments, the present disclosure provides a method for treating aging in a subject, comprising administering to the subject a therapeutically amount of the isolated protein in admixture with a pharmaceutically acceptable carrier. For example, the isolated protein may comprise an amino acid sequence selected from the group consisting of SEQ ID NO: 119-221, 329, 332, and 335. In various embodiments, the isolated protein further comprise a linker having the amino acid sequence set forth in SEQ ID NO: 44, a hinge linker having the amino acid sequence set forth in SEQ ID NO: 118, and a human IgG4 Fc (SEQ ID NO: 43). In some examples, the isolated protein is a fusion protein comprising a sequence of SEQ ID NO: 222.

In some embodiments, the pharmaceutical composition may comprise a plurality of the isolated proteins and at least 5%, 10%, 20%, 30%, 40%, 50%, 60%, 70%, 80%, or 90% of the isolated proteins are sialylated. For example, the pharmaceutical composition may comprise a plurality of the isolated proteins and at least 40% of the isolated proteins are sialylated. In some embodiments, the pharmaceutical composition may comprise a plurality of mutant soluble ActRIIA-ECD or ActRIIB-ECD proteins and at least 5%, 10%, 20%, 30%, 40%, 50%, 60%, 70%, 80%, or 90% of the mutant soluble ActRIIA-ECD or ActRIIB-ECD are sialylated at the position corresponding to N41 of SEQ ID NO: 1. Preferably, at least 40%, 50%, 60%, 70%, 80%, or 90% of the mutant soluble ActRIIA-ECD or ActRIIB-ECD in the pharmaceutical composition are sialylated at the position corresponding to N41 of SEQ ID NO: 1.

In some aspects, provided herein include a sample comprising a plurality of the isolated proteins and at least 5%, 10%, 20%, 30%, 40%, 50%, 60%, 70%, 80%, or 90% of the isolated proteins are sialylated. For example, the sample may comprise a plurality of the isolated proteins and at least 40% of the isolated proteins are sialylated. In some embodiments, the sample may comprise a plurality of mutant soluble ActRIIA-ECD or ActRIIB-ECD proteins and at least 5%, 10%, 20%, 30%, 40%, 50%, 60%, 70%, 80%, or 90% of the mutant soluble ActRIIA-ECD or ActRIIB-ECD are sialylated at the position corresponding to N41 of SEQ ID NO: 1. Preferably, at least 40%, 50%, 60%, 70%, 80%, or 90% of the mutant soluble ActRIIA-ECD or ActRIIB-ECD in the sample are sialylated at the position corresponding to N41 of SEQ ID NO: 1.

### Methods of Treatment and Therapeutic Uses

In one aspect, the present disclosure provides a method for treating myostatin-related or activin A-related disorders in a subject, comprising administering to the subject a therapeutically effective amount (either as monotherapy or in a combination therapy regimen) of the isolated protein of the present disclosure in pharmaceutically acceptable carrier. Importantly, the pharmaceutical compositions of the present disclosure can be used to increase lean muscle mass as a percentage of body weight and decrease fat mass as percentage of body weight, while avoiding safety concerns reported for existing ActRIIB-Fc fusion protein-based therapeutics.

In one aspect, the present disclosure provides a method of treating or preventing a muscle wasting in a subject, comprising administering to the subject a therapeutically effective amount (either as monotherapy or in a combination therapy regimen) of the isolated protein of the present disclosure in admixture with a pharmaceutically acceptable carrier, wherein such administration attenuates the loss of muscle mass and/or loss of muscle function. In various embodiments, the muscle wasting is associated with a disease selected from the group consisting of: muscular dystrophies (such as Duchenne muscular dystrophy (DMD), Becker MD, Limb-Girdle MD, Myotonic MD and Facioscapulohumeral muscular dystrophy (FSHD)), myositis (such as Dermatomyositis, Polymyositis and Inclusion body myositis), myopathy (including inherited myopathy as well as acquired myopathy such as myopathy induced by androgen-deprivation therapy, corticosteroids or statins), motoneuron disease (such as Lou Gehrig's Disease or ALS), spinal muscular atrophy (including Infantile progressive spinal muscular atrophy, Intermediate spinal muscular atrophy, Juvenile spinal muscular atrophy and Adult spinal muscular atrophy), neuromuscular junction disease (such as Myasthenia gravis, Lambert-Eaton syndrome and Botulism), peripheral nerve disease (such as Charcot-Marie tooth disease, Dejerine-Sottas disease and Friedreich's ataxia), spinal cord injury, stroke, neurodegenerative disease (including Parkinson's disease, Huntington's disease, Alzheimer's disease and Creutzfeldt-Jakob disease), cancer (such as lung cancer, pancreatic cancer, gastric cancer, colon cancer, prostate cancer, breast cancer, esophageal cancer, head and neck cancer, ovarian cancer, rhabdomyosarcoma, glioma, neuroblastoma, lymphoma, and multiple myeloma, skin cancer, and blood cancer), organ failure (such as heart failure, renal failure and liver failure, trauma (such as burns or motorcycle accident), disuse (such as long-term bed-rest, hospitalization, and spaceflight), infection (such as HIV, Polio and Sepsis), chronic obstructive pulmonary disease (COPD), and aging (such as sarcopenia, sarcopenic obesity and osteroarthritis).

In some examples, the myostatin-related or activin A-related disorder is selected from the group consisting of muscle wasting, a bone disorder, a metabolic disorder, and anemia. In some examples, the muscle wasting is associate with a condition selected from the group consisting of: muscular dystrophy, myositis, myopathy (e.g., critical illness myopathy, e.g., ICU myopathy), motorneuron disease, muscle atrophy, amyotrophic lateral sclerosis, spinal muscular atrophy, neuromuscular junction disease, peripheral nerve disease, spinal cord injury, stroke, neurodegenerative disease, anorexia, cancer, organ failure, trauma, disuse, infection, chronic obstructive pulmonary disease (COPD), sarcopenia, sarcopenic obesity, osteroarthritis, androgen deprivation, emphysema, cystic fibrosis, chronic heart failure, cardiac atrophy, cancer cachexia, renal failure, uremia, protein energy wasting, anorexia, malnutrition, sarcopenia, Acquired Immunodeficiency Syndrome (AIDS), sepsis, burn injury, diabetes, carpal tunnel syndrome, prolonged bed rest, bone fracture, aging, and exposure to microgravity. In some examples, the spinal muscular atrophy is selected from the group consisting of infantile progressive spinal muscular atrophy, intermediate spinal muscular atrophy, juvenile spinal muscular atrophy and adult spinal muscular atrophy. In some examples, the muscular dystrophy may be myotonic dystrophy. In one example, the myotonic dystrophy may be myotonic dystrophy type 1 (DM1). In another example, the myotonic dystrophy may be myotonic dystrophy type 2 (DM2). In some examples, the peripheral nerve disease is selected from the group consisting of Charcot-Marie Tooth disease, Dejerine-Sottas disease and Friedreich's ataxia. In some examples, the neurodegenerative disease is selected from the group consisting of Parkinson's disease, Huntington's disease, Alzheimer's disease and Creutzfeldt-Jakob disease. In some examples, the aging condition is selected from the group consisting of: frailty of the elderly, age-related sarcopenia, and osteoarthritis. In some examples, the motorneuron disease is amyotrophic lateral sclerosis (ALS or Lou Gehrig's Disease). In some examples, the bone disease is selected from the group consisting of: osteoporosis, renal osteodystrophy, osteomalacia, osteogenesis imperfecta, fibrodysplasia ossificans progressiva, corticosteroid-induced bone loss, androgen-deprivation therapy-induced bone loss, bone fracture, cancer-induced bone loss, bone metastasis, Paget's disease of the bone, Rickets, Perthes' disease and fibrous dysplasia.

In another aspect, the present disclosure provides a method of treating or preventing bone disease in a subject, comprising administering to the subject a therapeutically effective amount (either as monotherapy or in a combination therapy regimen) of an isolated protein of the present disclosure in admixture with a pharmaceutically acceptable carrier. In various embodiments, the bone disease is selected from the group consisting of: osteoporosis, renal osteodystrophy, osteogenesis imperfecta, fibrodysplasia ossificans progressiva, corticosteroid-induced bone loss, androgen-depriviation therapy-induced bone loss, hip fracture, cancer-induced bone loss, bone metastasis, Paget's disease, Rickets, osteomalacia, Perthes' disease and fibrous dysplasia.

In another aspect, the present disclosure provides a method of treating or preventing a metabolic disorder in a subject, comprising administering to the subject a therapeutically effective amount (either as monotherapy or in a combination therapy regimen) of an isolated protein of the present disclosure in admixture with a pharmaceutically acceptable carrier. In various embodiments, the metabolic disorder is selected from the group consisting of: metabolic syndrome, obesity, dyslipidemia, sarcopenic obesity, non-alcoholic fatty liver disease such as non-alcoholic steatohepatitis (NASH), alcoholic fatty liver disease, insulin resistance, diabetes as well as diabetic myopathy, diabetic nephropathy, diabetic neuropathy, diabetic retinopathy, and hemochromatosis.

In another aspect, the present disclosure provides a method of treating or preventing fibrosis in a subject, comprising administering to the subject a therapeutically effective amount (either as monotherapy or in a combination therapy regimen) of an isolated protein of the present disclosure in admixture with a pharmaceutically acceptable carrier. In various embodiments, the fibrosis is selected from the group consisting of: interstitial lung disease, idiotypic pulmonary fibrosis, cystic fibrosis, liver fibrosis, cirrhosis, biliary atresia, myocardial infarction, cardiac fibrosis, renal fibrosis, myelofibrosis, idiopathic retroperitoneal fibrosis, nephrogenic fibrosing dermopathy, inflammatory bowel disease or Crohn's disease, keloid, scleroderma, retroperitoneal fibrosis, and arthrofibrosis.

In another aspect, the present disclosure provides a method of treating or preventing an autoimmune/inflammatory disease in a subject, comprising administering to the subject a therapeutically effective amount (either as monotherapy or in a combination therapy regimen) of an isolated protein of the present disclosure in admixture with a pharmaceutically acceptable carrier. In various embodiments, the disease is selected from the group consisting of: autoimmune/inflammatory disorders including multiple sclerosis (MS), systemic sclerosis, diabetes (type-1), glomerulonephritis, myasthenia gravis, psoriasis, systemic lupus erythematosus, polymyositis, Crohn's disease, ulcerative colitis, and primary biliary cirrhosis, arthritis, asthma, and sepsis.

In another aspect, the present disclosure provides a method of treating cardiovascular disease in a subject, comprising administering to the subject a therapeutically effective amount (either as monotherapy or in a combination therapy regimen) of an isolated protein of the present disclosure in admixture with a pharmaceutically acceptable carrier. In various embodiments, the cardiovascular disease is selected from the group consisting of: heart failure, cardiac atrophy, pulmonary arterial hypertension (PAH), myocarditis, coronary artery disease, myocardial infarction, cardiac arrhythmias, heart valve disease, cardiomyopathy, pericardial disease, aorta disease, Marfan syndrome and cardiac transplant.

In another aspect, the present disclosure provides for a method of treating cardiac dysfunction or heart failure in a subject comprising administering an effective amount of an isolated protein into the subject. The modulation may improve cardiac function of the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. The improvement of cardiac function can be evaluated by echocardiography to measure 1) cardiac pump functions focusing on the ejected blood volume and the efficiency of ejection and 2) myocardial functions focusing on the strength of myocardial contraction.

In another aspect, the present disclosure provides for a method of treating cancer cells in a subject, comprising administering to the subject a therapeutically effective amount (either as monotherapy or in a combination therapy regimen) of an isolated protein of the present disclosure in pharmaceutically acceptable carrier, wherein such administration inhibits the growth and/or proliferation of a cancer cell. Specifically, an isolated protein of the present disclosure is useful in treating disorders characterized as cancer. Such disorders include, but are not limited to solid tumors, such as cancers of the breast, respiratory tract, brain, reproductive organs, digestive tract, urinary tract, eye, liver, skin, head and neck, thyroid, parathyroid and their distant metastases, lymphomas, sarcomas, multiple myeloma and leukemia. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to small-cell and non-small-cell lung carcinoma, as well as bronchial adenoma and pleuropulmonary blastoma. Examples of brain cancers include, but are not limited to brain stem and hypophthalmic glioma, cerebellar and cerebral astrocytoma, medulloblastoma, ependymoma, as well as neuroectodermal and pineal tumor. Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, pancreatic, rectal, small-intestine, and salivary gland cancers. Tumors of the urinary tract include, but are not limited to bladder, penile, kidney, renal pelvis, ureter, and urethral cancers. Eye cancers include, but are not limited to intraocular melanoma and retinoblastoma. Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to nasopharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia. In certain embodiments, the cancer will be a cancer with high expression of TGF-B family member, such as activin A, myostatin, TGF-β and GDF15, e.g., pancreatic cancer, gastric cancer, ovarian cancer, colorectal cancer, melanoma leukemia, lung cancer, prostate cancer, brain cancer, bladder cancer, and head-neck cancer.

In another aspect, the present disclosure provides for a method of treating chronic kidney disease (CKD) in a subject, comprising administering to the subject a therapeutically effective amount (either as monotherapy or in a combination therapy regimen) of an isolated protein of the present disclosure in pharmaceutically acceptable carrier, wherein such administration attenuates the loss of muscle mass and/or loss of muscle function or inhibits kidney fibrosis. Specifically, an isolated protein of the present disclosure is useful in treating CKD including renal failure, interstitial fibrosis, and kidney dialysis as well as protein energy wasting (PEW) associated with CKD. The modulation may improve CKD or PEW of the subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. The improvement of renal function can be evaluated by measuring protein/creatinine ratio (PCR) in the urine and glomerular filtration rate (GFR). Improvement of PEW can be evaluated by measuring serum levels of albumin and inflammatory cytokines, rate of protein synthesis and degradation, body mass, muscle mass, physical activity and nutritional outcomes.

In another aspect, the present disclosure provides for methods for treating arthritis in a subject, comprising administering to the subject a therapeutically effective amount (either as monotherapy or in a combination therapy regimen) of an isolated protein of the present disclosure in pharmaceutically acceptable carrier. Specifically, an isolated protein of the present disclosure is useful in treating an arthritis selected from rheumatoid arthritis and osteoarthritis.

In another aspect, the present disclosure provides for methods for treating anorexia in a subject, comprising administering to the subject a therapeutically effective amount (either as monotherapy or in a combination therapy regimen) of an isolated protein of the present disclosure in pharmaceutically acceptable carrier. Specifically, an isolated protein of the present disclosure is useful in treating an anorexia selected from anorexia nervosa and anorexia-cachexia syndrome.

In another aspect, the present disclosure provides for methods for treating liver disease in a subject, comprising administering to the subject a therapeutically effective amount (either as monotherapy or in a combination therapy regimen) of an isolated protein of the present disclosure in pharmaceutically acceptable carrier. Specifically, an isolated protein of the present disclosure is useful in treating a liver disease selected from non-alcoholic fatty liver disease, non-alcoholic steatohepatitis, alcoholic fatty liver disease, liver cirrhosis, liver failure, autoimmune hepatitis and hepatocellular carcinoma.

In another aspect, the present disclosure provides for methods for organ or tissue transplantation in a subject, comprising administering to the subject a therapeutically effective amount (either as monotherapy or in a combination therapy regimen) of an isolated protein of the present disclosure in pharmaceutically acceptable carrier. Specifically, an isolated protein of the present disclosure is useful in treating a transplantation selected from organ transplantations of the heart, kidneys, liver, lungs, pancreas, intestine and thymus or from tissues transplantations of the bones, tendons, cornea, skin, heart valves, nerves and veins.

In another aspect, the present disclosure provides for methods for treating anemia in a subject, comprising administering to the subject a therapeutically effective amount (either as monotherapy or in a combination therapy regimen) of an isolated protein of the present disclosure in pharmaceutically acceptable carrier. In various embodiments, the anemia is selected from various anemia disorders including cancer-associated anemia, chemotherapy-induced anemia, chronic kidney disease-associated anemia, iron-deficiency anemia, thalassemia, sickle cell disease, aplastic anemia and myelodysplastic syndromes.

In another aspect, the present disclosure provides methods of treating pain in a subject, comprising administering a therapeutically effective amount of the pharmaceutical compositions of the invention to a subject in need thereof. In one embodiment, the subject is a human subject. In various embodiments, the pain is selected from neuropathic pain, inflammatory pain, or cancer pain.

In another aspect, the present disclosure provides a method of treating aging in a subject, comprising administering to the subject a therapeutically effective amount (either as monotherapy or in a combination therapy regimen) of an isolated protein of the present disclosure in pharmaceutically acceptable carrier. In various embodiments, the aging condition is selected from the group consisting of: frailty of the elderly, age-related sarcopenia, and osteoarthritis.

In another aspect, the present disclosure provides methods of inducing stem cell growth for tissue repair or organ regeneration in a subject, comprising administering to the subject a therapeutically effective amount (either as monotherapy or in a combination therapy regimen) of an isolated protein of the present disclosure in pharmaceutically acceptable carrier. In various embodiments, the stem cell is selected from the group consisting of: muscle stem (satellite) cell, cardiac stem cell, bone marrow-derived mesynchymal stem cell and pluripotent stem cell.

In various embodiments, the present disclosure provides for a method of inhibiting loss of muscle mass and/or muscle function in a subject comprising administering an effective amount of an isolated protein into the subject. The modulation may attenuate the loss of the muscle mass and/or function of the subject by at least 5%, 10%, at least 25%, at least 50%, at least 75%, or at least 90%. The inhibition of loss of muscle mass and function can be evaluated by using imaging techniques and physical strength tests. Examples of imaging techniques for muscle mass evaluation include Dual-Energy X-Ray Absorptiometry (DEXA), Magnetic Resonance Imaging (MRI), and Computed Tomography (CT). Examples of muscle function tests include grip strength test, stair climbing test, short physical performance battery (SPPB) and 6-minute walk, as well as maximal inspiratory pressure (MIP) and maximal expiratory pressure (MEP) that are used to measure respiratory muscle strength.

“Therapeutically effective amount” or “therapeutically effective dose” refers to that amount of the therapeutic agent being administered which will relieve to some extent one or more of the symptoms of the disorder being treated.

A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful doses in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

An exemplary, non-limiting daily dosing range for a therapeutically or prophylactically effective amount of an isolated protein of the disclosure can be 0.001 to 100 mg/kg, 0.001 to 90 mg/kg, 0.001 to 80 mg/kg, 0.001 to 70 mg/kg, 0.001 to 60 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the conditions to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In various embodiments, the total dose administered will achieve a plasma antibody concentration in the range of, e.g., about 1 to 1000 μg/ml, about 1 to 750 μg/ml, about 1 to 500 μg/ml, about 1 to 250 μg/ml, about 10 to 1000 μg/ml, about 10 to 750 μg/ml, about 10 to 500 μg/ml, about 10 to 250 μg/ml, about 20 to 1000 μg/ml, about 20 to 750 μg/ml, about 20 to 500 μg/ml, about 20 to 250 μg/ml, about 30 to 1000 μg/ml, about 30 to 750 μg/ml, about 30 to 500 μg/ml, about 30 to 250 μg/ml.

Toxicity and therapeutic index of the pharmaceutical compositions of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effective dose is the therapeutic index and it can be expressed as the ratio LD50/ED50. Compositions that exhibit large therapeutic indices are generally preferred.

The dosing frequency of the administration of the isolated protein pharmaceutical composition depends on the nature of the therapy and the particular disease being treated. The subject can be treated at regular intervals, such as weekly or monthly, until a desired therapeutic result is achieved. Exemplary dosing frequencies include, but are not limited to: once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Combination Therapy

The isolated proteins and related compositions according to the present disclosure may be used in combination therapies. As used herein, the terms “co-administration”, “co-administered” and “in combination with”, referring to the an isolated protein of the disclosure and one or more other therapeutic agents, is intended to mean, and does refer to and include the following: simultaneous administration of such combination of an isolated protein of the disclosure and therapeutic agent(s) to a subject in need of treatment, when such components are formulated together into a single dosage form which releases said components at substantially the same time to said subject; substantially simultaneous administration of such combination of an isolated protein of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of an isolated protein of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of an isolated protein of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

In another aspect, the present disclosure relates to methods of treating muscle wasting diseases in a subject, comprising administration of a combination of a) a therapeutically effective amount of an isolated protein of the present disclosure; and b) a second agent. This combination therapy may be particularly effective against a muscle wasting disease that is resistant or refractory to treatment using the second agent alone. In various embodiments, second agent is selected from growth hormone, ghrelin, IGF1, insulin, prednisone, corticosteroid therapy, androgen-deprivation therapy, anabolic steroids, antagonists to angiotensin or angiotensin receptor, antagonists to inflammatory cytokines such as TNF-alpha, IL-6, IL-1 and their receptors, and other antagonists to myostatin, activin A or another member of the TGF-beta family and to their receptors, bisphosphonates, RANKL inhibitors, agonists of peroxisome proliferator-activated receptors, β2 agonists, activator of PGC-1alpha, proteasome inhibitors, cancer therapeutics, chemotherapeutic agents, cell therapy, stem cell therapy, gene therapy, gene targeting therapy, and antisense oligonucleotide therapy.

The second agent composition may be administered to the subject prior to, concurrently with, or subsequent to administration of the pharmaceutical composition comprising the isolated protein. In various embodiments, the combination therapy comprises administering an isolated protein and the second agent composition simultaneously, either in the same pharmaceutical composition or in separate pharmaceutical composition. In various embodiments, a pharmaceutical composition comprising the isolated protein and the second agent may be administered sequentially, e.g., the pharmaceutical composition is administered either prior to or after the administration of the second agent composition.

In various embodiments, the administrations of an isolated protein composition and the second agent composition are concurrent, e.g., the administration period of an isolated protein composition and the second agent composition overlap with each other.

In various embodiments, the administrations of an isolated protein composition and the second agent composition are non-concurrent. For example, in various embodiments, the administration of an isolated protein composition is terminated before the second agent composition is administered. In various embodiments, the administration second agent composition is terminated before an isolated protein composition is administered.

### EXAMPLES

### Example 1

In this example, the preparation of an isolated protein comprising a mutant soluble ActRIIB-ECD polypeptide fused with an Fc domain is generally described. The mutant soluble ActRIIB-ECD polypeptide was designed by substituting the asparagine corresponding to position N18 of SEQ ID NO: 29 with a glutamine (Q).

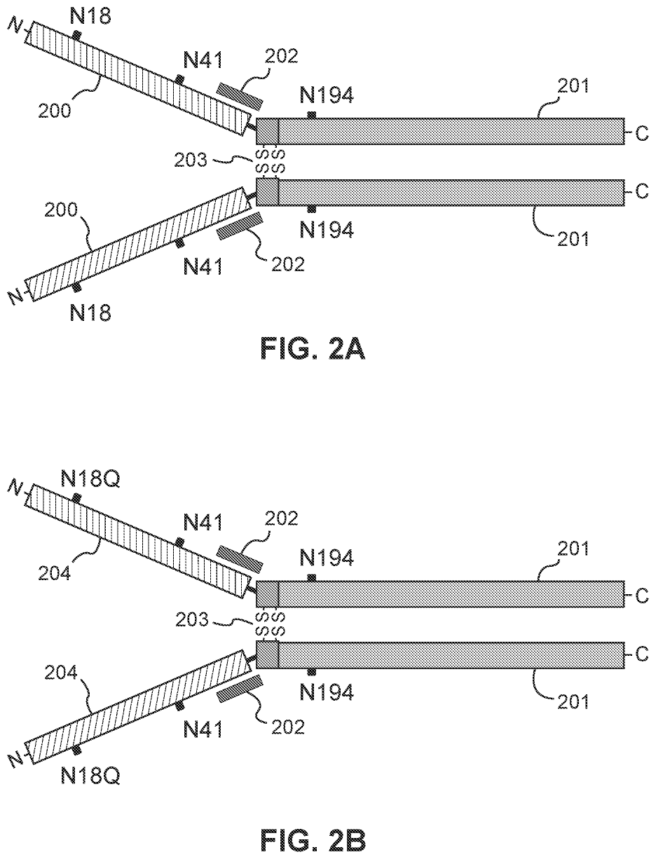

The isolated protein is a construct as depicted in FIG. 2B. As shown in FIG. 2B, the protein may form a dimer of two monomers. Each monomer prepared in this example comprises a mutant soluble ActRIIB-ECD (SEQ ID NO: 146, which comprises the N18Q mutation compared to the soluble ActRIIB-ECD of SEQ ID NO: 29), a peptide linker sequence, a hinge linker sequence, and an Fc domain. The construct has three O-linked glycosylation sites and two N-linked glycosylation sites (at N41 and N194) with the glycosylation site at amino acid 18 removed by the N18Q mutation. The construct in FIG. 2B has a N18Q mutation compared to the construct in FIG. 2A, which has three N-linked glycosylation sites, at N18, N41, and N194.

DNA expression cassette encoding the mutant soluble ActRIIB-ECD polypeptide (SEQ ID NO: 146) was synthesized as a double stranded Gene fragment by IDT (Integrated DNA Technologies). Human Fc cassette (encoding SEQ ID NO: 43) was PCR amplified from its DNA template and the two DNA cassettes pieces were cloned into a mammalian expression vector using HIFI GIBSON Assembly master mix (New England Biolabs). The resulting construct encodes a fusion protein comprising a signal peptide leader sequence (SEQ ID NO: 49), a mutant soluble ActRIIB-ECD polypeptide (SEQ ID NO: 146), a peptide linker sequence (SEQ ID NO: 44), a hinge linker sequence (SEQ ID NO: 118), and an Fc domain sequence (SEQ ID NO: 43). The DNA sequence encoding this fusion protein comprises SEQ ID NO: 1652.

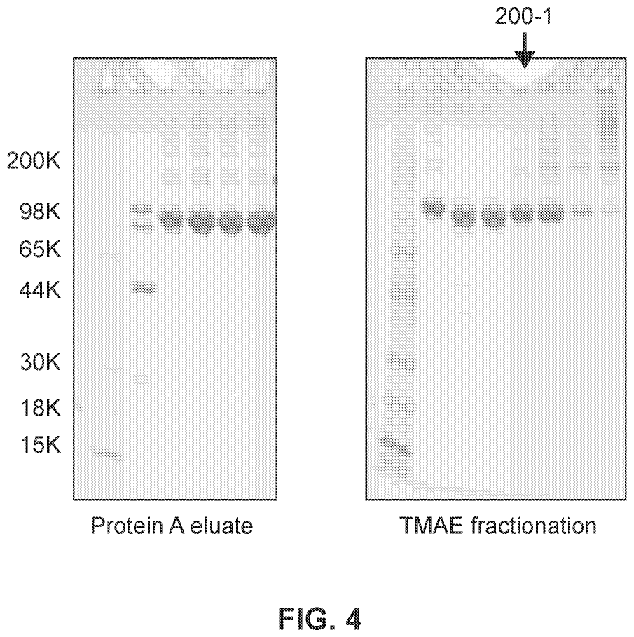

This plasmid was transfected into CHO cells using a lipid based transfection reagent. The transfected cells were grown in a culture medium. The fusion protein was secreted to the medium, which was collected for protein purification. The supernatants containing the secreted ActRIIB-huFc fusion protein were isolated by centrifuging and passing through a PES filter. The fusion protein was purified from the clarified medium by sequential chromatography steps on Protein A and Fractogel EMC TMAE anion exchange chromatography columns. FIG. 4 shows SDS-PAGE analysis of the column fractions. Fractionation 200-1 accounts for approximately 75% of the load. The purified protein runs as dimer as illustrated in FIG. 2B with a molecular weight of ˜90 kDa. Fraction 200-1 had the most of purified protein and was used for further testing (e.g., by the assays described in Example 3). The resulting ActRIIB-huFc fusion protein comprises the sequence of SEQ ID NO: 222, and generally forms dimers as illustrated in FIG. 2B. The mature fusion proteins generally lack the signal peptide leader sequence.

### Example 2

In this example, the preparation of an isolated protein comprising a mutant soluble ActRII-ECD polypeptide fused with an Fc domain is generally described. The mutant soluble ActRII-ECD polypeptide is designed by substituting the asparagine corresponding to position N18 of SEQ ID NO: 1 with glutamine. Besides the N18Q mutation, the mutant soluble ActRII-ECD polypeptide may comprise one or more additional mutations described in the disclosure.

The isolated protein is one of the constructs depicted in FIG. 2B. As shown in FIG. 2B, the protein may form a dimer of two monomers. Each monomer comprises a mutant soluble ActRII-ECD (with at least an N18Q mutation compared to the soluble ActRIIB-ECD of SEQ ID NO: 1 or the soluble ActRIIA-ECD of SEQ ID NO: 1654), a peptide linker sequence, a hinge linker sequence, and an Fc domain. The construct has three O-linked glycosylation sites and two N-linked glycosylation sites (at N41 and N194) with the glycosylation site at amino acid 18 removed by the N18Q mutation. The construct in FIG. 2B has a N18Q mutation compared to the construct in FIG. 2A, which has three N-linked glycosylation sites, at N18, N41, and N194.

DNA expression cassette encoding the mutant soluble ActRIIB-ECD polypeptide (e.g., any one of SEQ ID NO: 119, 120-145, 147-221, 329, 332, or 335) or mutant soluble ActRIIA-ECD polypeptide (e.g., any one of SEQ ID NO: 1655, 1656, or 1657) is synthesized as a double stranded Gene fragment by IDT (Integrated DNA Technologies). Human Fc cassette (encoding an Fc domain (e.g., any one of SEQ ID NO: 39, 41, or 43)) is PCR amplified from its DNA template and the two DNA cassettes pieces are cloned into a mammalian expression vector using HIFI GIBSON Assembly master mix (New England Biolabs). The resulting construct generates a fusion protein comprising a signal peptide leader sequence (e.g., SEQ ID NO: 49), a mutant soluble ActRII-ECD polypeptide (e.g., any one of SEQ ID NO: 119, 120-145, 147-221, 329, 332, 335, 1655, 1656, or 1657), a peptide linker sequence (SEQ ID NO: 44), a hinge linker sequence (SEQ ID NO: 118) and an Fc domain sequence (e.g., any one of SEQ ID NO: 39, 41, or 43).

This plasmid is transfected into CHO cells using a lipid based transfection reagent. This plasmid is transfected into CHO cells using a lipid based transfection reagent. The transfected cells are grown in a culture medium. The fusion protein is secreted to the medium, which is collected for protein purification. The supernatants containing the secreted ActRIIB-huFc fusion protein is isolated by centrifuging and passing through a PES filter. The fusion protein is purified from the clarified medium by sequential chromatography steps on Protein A and Fractogel EMC TMAE anion exchange chromatography columns. The purified protein runs as dimer as illustrated in FIG. 2B with a molecular weight of ˜90 kDa.

### Example 3

In this example, the myostatin binding activities of mutant ActRIIB polypeptide fusion proteins is evaluated.

Myostatin binding activities of mutant ActRIIB polypeptide fusion protein with N18Q mutation (SEQ ID NO: 119) as well as the corresponding ActRIIB polyppetide fusion protein without N18Q mutation (SEQ ID NO: 29) were analyzed using an ELISA assay. Results from the analysis are shown in Table 8 below. Batches of ActRIIB polypeptide fusion protein comprising soluble ActRIIB-ECD without the N18Q mutation (Sample #1 (used as reference when calculating potency), Sample #2, and #3) had varying levels of sialylation at the N18 position, and the inventors discovered that the soluble ActRIIB-ECD fusion proteins showed a reduction in potency with increased levels of sialylation. In contrast, the fusion protein with N18Q mutation in the soluble ActRIIB-ECD polypeptide produced in Example 1 (i.e., with SEQ ID NO: 222) (Sample #4) showed greater potency (e.g., myostatin binding) than the reference batch (Sample #1) and greater potency than either of the more highly sialylated batches (Samples #2 and #3). Percent sialylation was determined by mass spectrometry of the N18 peptide.

TABLE 8

| Sample | | N18 | Potency % of |
|---|---|---|---|
| # | Test sample | (% sialylation) | reference |
| 1 | Reference | 20 | 100 |
| 2 | Batch 2 | 75 | 79 |
| 3 | Batch 3 | 83 | 57 |
| 4 | N18Q mutant | 0 | 108 |

### Example 4

In this example, the myostatin binding activities of mutant ActRIIB and mutant ActRIIA polypeptide fusion proteins is evaluated. Without wishing to be bound by theory, it is expected that the absence of the glycosylation site corresponding to position N18 of SEQ ID NO: 1 reduces steric hinderance in the myostatin binding site of ActRII polypeptide fusion proteins. Accordingly, the inventors envision that removal of the N18 glysosylation site from various mutant ActRIIA and ActRIIB polypeptide fusion proteins will result in similar improvements to myostatin binding affinity.

Myostatin binding activities of various mutant ActRIIA and mutant ActRIIB polypeptide fusion proteins with N18Q mutation (e.g., fusion proteins comprising mutant soluble ActRIIB-ECD polypeptides comprising SEQ ID NOs: 119, 120-145, 147-221, 329, 332, or 335 or mutant soluble ActRIIA-ECD polypeptides comprising, e.g., SEQ ID NO: 1655, 1656, or 1675) as well as corresponding ActRIIB polypeptide fusion proteins without N18Q are analyzed using an ELISA assay. The mutant ActRIIA and mutant ActRIIB polypeptide fusion proteins with N18Q mutation are expected to display increased binding affinity for myostatin relative to the corresponding fusion proteins that lack the N18Q mutation (i.e., include a N-linked glycosylation site at position N18).

TABLE 9
Examples of sequences

| SEQ | | |
|---|---|---|
| ID | | |
| NO: | Notes | Sequences |
| 1 | Truncated wild- | ETRECIYYNANWELERTNQSGLERCEGEQDKRLHCYASWRNS |
| | type ActRIIB-ECD | SGTIELVKKGCWLDDFNCYDRQECVATEENPQVYFCCCEGNF |
| | (amino acids 25- | CNERFTHLPEAGGPEVTYEPPPTAPT |
| | 134 of SEQ ID NO: | |
| | 45) | |
| 45 | Full Length Amino | MTAPWVALALLWGSLCAGSGRGEAETRECIYYNANWELERT |
| | Acid Sequence of | NQSGLERCEGEQDKRLHCYASWRNSSGTIELVKKGCWLDDFN |
| | Human ActRIIB | CYDRQECVATEENPQVYFCCCEGNFCNERFTHLPEAGGPEVTY |
| | polypeptide | EPPPTAPTLLTVLAYSLLPIGGLSLIVLLAFWMYRHRKPPYGHV |
| | | DIHEDPGPPPPSPLVGLKPLQLLEIKARGRFGCVWKAQLMNDF |
| | | VAVKIFPLQDKQSWQSEREIFSTPGMKHENLLQFIAAEKRGSNL |
| | | EVELWLITAFHDKGSLTDYLKGNIITWNELCHVAETMSRGLSY |
| | | LHEDVPWCRGEGHKPSIAHRDFKSKNVLLKSDLTAVLADFGL |
| | | AVRFEPGKPPGDTHGQVGTRRYMAPEVLEGAINFQRDAFLRID |
| | | MYAMGLVLWELVSRCKAADGPVDEYMLPFEEEIGQHPSLEEL |
| | | QEVVVHKKMRPTIKDHWLKHPGLAQLCVTIEECWDHDAEAR |
| | | LSAGCVEERVSLIRRSVNGTTSDCLVSLVTSVTNVDLPPKESSI |
| 46 | Wild-type human | SGRGEAETRECIYYNANWELERTNQSGLERCEGEQDKRLHCY |
| | ActRIIB | ASWRNSSGTIELVKKGCWLDDFNCYDRQECVATEENPQVYFC |
| | extracellular | CCEGNFCNERFTHLPEAGGPEVTYEPPPTAPT |
| | domain (amino | |
| | acids 19-134 of | |
| | SEQ ID NO: 45) | |
| 47 | Full Length Amino | MGAATKLAFAVFLISCSSGAILGRSETQECIYYNANWEKDKTN |
| | Acid Sequence of | RSGIEPCYGDKDKRRHCFATWKNISGSIEIVKQGCWLDDINCY |
| | Human ActRIIA | DRNDCIEKKDSPEVFFCCCEGNMCNERFFYFPEMEVTQPTSNP |
| | polypeptide | VTPKPPLFNTLLYSLVPIMGIAVIVLFSFWMYRHHKLAYPPVLV |
| | | PTQDPGPPPPSPLMGLKPLQLLEIKARGRFGCVWKAQLLNEYV |
| | | AVKIFPIQDKQSWQNEYEIYSLPGMKHDNILQFIGAEKRGTSID |
| | | VDLWLITAFHEKGSLTDFLKANVVSWNELCHIAQTMARGLAY |
| | | LHEDIPGLKDGHKPAISHRDIKSKNVLLKNNLTACIADFGLALK |
| | | FEAGKSAGDTHGQVGTRRYMAPEVLEGAINFQRDAFLRIDMY |
| | | AMGLVLWELASRCTASDGPVDEYMLPFEEEIGQHPSLEDMQE |
| | | VVVHKKKRPVLRECWQKHSGMAMLCETIEECWDHDAEARLS |
| | | AGCVEERIIQMQKLTNIITTEDIVTVVTMVTNVDFPPKESSL |
| 48 | Wild-type human | AILGRSETQECLFFNANWEKDRTNQTGVEPCYGDKDKRRHCF |
| | ActRIIA | ATWKNISGSIEIVKQGCWLDDINCYDRTDCVEKKDSPEVYFCC |
| | extracellular | CEGNMCNEKFSYFPEMEVTQPTSNPVTPKPP |
| | domain (20-135 of | |
| | SEQ ID NO: 47) | |
| 119 | N18Q mutant of | ETRECIYYNANWELERTQQSGLERCEGEQDKRLHCYASWRNS |
| | truncated wild type | SGTIELVKKGCWLDDFNCYDRQECVATEENPQVYFCCCEGNF |
| | soluble ActRIIB- | CNERFTHLPEAGGPEVTYEPPPTAPT |
| | ECD (SEQ ID NO: | |
| | 1) | |
| 223 | N18X mutant of | ETRECIYYNANWELERTXQSGLERCEGEQDKRLHCYASWRNS |
| | wild type soluble | SGTIELVKKGCWLDDFNCYDRQECVATEENPQVYFCCCEGNF |
| | ActRIIB-ECD | CNERFTHLPEAGGPEVTYEPPPTAPT |
| | (SEQ ID NO: 1) | Wherein X is any amino acid that is not N |
| 2 | Truncated wild- | ETQECLFFNANWEKDRTNQTGVEPCYGDKDKRRHCFATWKNI |
| | type ActRIIA-ECD | SGSIEIVKQGCWLDDINCYDRTDCVEKKDSPEVYFCCCEGNMC |
| | | NEKFSYFPEMEVTQPTSNPVTPKPP |
| 3 | Hybrid hu- | ETRECIYYNANWELERTNQSGLERCYGDKDKRRHCYASWRNS |
| | ActRIIB-ECD | SGTIELVKKGCWLDDINCYDRTDCVEKKDSPEVYFCCCEGNFC |
| | (AG-0001) | NERFTHLPEAGGPEVTYEPPPTAPT |
| 4 | Hybrid hu- | ETRECIYYNANWELERTNQSGLERCYGDKDKRRHCYASWRNS |
| | ActRIIB-ECD | SGTIELVKKGCWLDDFNCYDRTDCVEKKDSPEVYFCCCEGNF |
| | (AG-0002) | CNERFTHLPEAGGPEVTYEPPPTAPT |
| 5 | Hybrid hu- | ETRECIYYNANWELERTNQSGLERCEGEQDKRLHCYASWRNS |
| | ActRIIB-ECD | SGTIELVKKGCWLDDINCYDRTDCVEKKDSPEVYFCCCEGNFC |
| | (AG-0003) | NERFTHLPEAGGPEVTYEPPPTAPT |
| 6 | Hybrid hu- | ETRECIYYNANWELERTNQSGLERCEGEQDKRLHCYASWRNS |
| | ActRIIB-ECD | SGTIELVKKGCWLDDINCYDRTDCVEKKDSPQVYFCCCEGNFC |
| | (AG-0004) | NERFTHLPEAGGPEVTYEPPPTAPT |
| 7 | Hybrid hu- | ETRECIYYNANWELERTNQSGLERCEGEQDKRLHCYASWRNS |
| | ActRIIB-ECD | SGTIELVKKGCWLDDFNCYDRTDCVEKKDSPQVYFCCCEGNF |
| | | CNERFTHLPEAGGPEVTYEPPPTAPT |
| 8 | Hybrid hu- | ETRECIYYNANWELERTNQSGLERCEGEQDKRLHCYASWRNS |
| | ActRIIB-ECD | SGTIELVKKGCWLDDFNCYDRTDCVEKKDSPEVYFCCCEGNF |
| | (AG-0006) | CNERFTHLPEAGGPEVTYEPPPTAPT |
| 9 | Hybrid hu- | ETRECIYYNANWELERTNQSGLERCEGEQDKRLHCYASWRNS |
| | ActRIIB-ECD | SGTIELVKKGCWLDDFNCYDRQECVEKKDSPEVYFCCCEGNF |
| | (AG-0007) | CNERFTHLPEAGGPEVTYEPPPTAPT |
| 10 | Hybrid hu- | ETRECIYYNANWELERTNQSGLERCEGEQDKRLHCYASWRNS |
| | ActRIIB-ECD | SGTIELVKKGCWLDDFNCYDRQECVEKKDSPQVYFCCCEGNF |
| | (AG-0008) | CNERFTHLPEAGGPEVTYEPPPTAPT |
| 11 | Hybrid human | TRECIYYNANWELERTNQSGLERCEGEQDKRLHCYASWRNSS |
| | ActRIIA-ECD | GTIELVKKGCWLDDINCYDRQECVEKKDSPEVYFCCCEGNFC |
| | (AG-0009) | NERFTHLPEAGGPEVTYEPPPTAPT |
| 12 | Hybrid hu- | ETRECIYYNANWELERTNQSGLERCEGEQDKRLHCYASWRNS |
| | ActRIIB-ECD | SGTIELVKKGCWLDDFNCYDRTDCVEKKDSPEVYFCCCEGNM |
| | (AG-0010) | CNERFTHLPEAGGPEVTYEPPPTAPT |
| 13 | Hybrid hu- | ETRECIYYNANWELERTNQSGLERCEGDQDKRLHCYASWRNS |
| | ActRIIB-ECD | SGTIELVKKGCWLDDFNCYDRQECVEKKDSPEVYFCCCEGNM |
| | (AG-0011) | CNERFTHLPEAGGPEVTYEPPPTAPT |
| 14 | Hybrid hu- | ETQECIYYNANWEKDRTNQTGVEPCYGDKDKRRHCYASWRN |
| | ActRIIB-ECD | SSGTIELVKKGCWLDDFNCYDRQECVATEENPQVYFCCCEGN |
| | (AG-0012) | FCNERFTHLPEAGGPEVTYEPPPTAPT |
| 15 | Hybrid hu- | ETQECIYYNANWEKDRTNQTGVEPCEGDQDKRLHCYASWRN |
| | ActRIIB-ECD | SSGTIELVKKGCWLDDFNCYDRQECVATEENPQVYFCCCEGN |
| | (AG-0013) | FCNERFTHLPEAGGPEVTYEPPPTAPT |
| 16 | Hybrid hu- | ETRECIYYNANWELERTNQSGLERCYGDKDKRRHCYASWRNS |
| | ActRIIB-ECD | SGTIELVKKGCWLDDFNCYDRQECVATEENPQVYFCCCEGNF |
| | (AG-0014) | CNERFTHLPEAGGPEVTYEPPPTAPT |
| 17 | Hybrid hu- | ETRECIYYNANWEKDRTNQTGVEPCEGDQDKRLHCYASWRN |
| | ActRIIB-ECD | SSGTIELVKKGCWLDDFNCYDRQECVATEENPQVYFCCCEGN |
| | (AG-0015) | FCNERFTHLPEAGGPEVTYEPPPTAPT |
| 18 | Hybrid hu- | ETQECIYYNANWEKDRTNQTGVEPCEGDQDKRLHCYASWRN |
| | ActRIIB-ECD | SSGTIELVKKGCWLDDFNCYDRQECVATEENPQVYFCCCEGN |
| | (AG-0016) | FCNERFTHLPEAGGPEVTYEPPPTAPT |
| 19 | Hybrid hu- | ETQECIYYNANWEKDRTNQTGLERCEGEQDKRLHCYASWRNS |
| | ActRIIB-ECD | SGTIELVKKGCWLDDFNCYDRQECVATEENPQVYFCCCEGNF |
| | (AG-0017) | CNERFTHLPEAGGPEVTYEPPPTAPT |
| 20 | Hybrid hu- | ETQECIYYNANWEKDRTNQSGLERCEGEQDKRLHCYASWRNS |
| | ActRIIB-ECD | SGTIELVKKGCWLDDFNCYDRQECVATEENPQVYFCCCEGNF |
| | (AG-0018) | CNERFTHLPEAGGPEVTYEPPPTAPT |
| 21 | Hybrid hu- | ETRECIYYNANWEKDRTNQSGLERCEGEQDKRLHCYASWRNS |
| | ActRIIB-ECD | SGTIELVKKGCWLDDFNCYDRQECVATEENPQVYFCCCEGNF |
| | (AG-0019) | CNERFTHLPEAGGPEVTYEPPPTAPT |
| 22 | Hybrid hu- | ETQECIYYNANWELERTNQSGLERCEGEQDKRLHCYASWRNS |
| | ActRIIB-ECD | SGTIELVKKGCWLDDFNCYDRQECVATEENPQVYFCCCEGNF |
| | (AG-0020) | CNERFTHLPEAGGPEVTYEPPPTAPT |
| 23 | Hybrid hu- | ETRECIYYNANWELERTNQSGLERCEGEQDKRLHCFATWRNS |
| | ActRIIB-ECD | SGTIELVKQGCWLDDFNCYDRQECVATEENPQVYFCCCEGNF |
| | (AG-0021) | CNERFTHLPEAGGPEVTYEPPPTAPT |
| 24 | Hybrid hu- | ETRECIYYNANWELERTNQSGLERCYGDKDKRRHCYASWRNS |
| | ActRIIB-ECD | SGTIELVKKGCWLDDINCYDRQECVATEENPQVYFCCCEGNFC |
| | (AG-0022) | NERFTHLPEAGGPEVTYEPPPTAPT |
| 25 | Hybrid hu- | ETRECIYYNANWELERTNQSGLERCEGEQDKRLHCYASWRNS |
| | ActRIIB-ECD | SGTIELVKKGCWLDDFNCYDRQECVATKENPQVYFCCCEGNF |
| | (AG-0023) | CNERFTHLPEAGGPEVTYEPPPTAPT |
| 26 | Hybrid hu- | ETRECIYYNANWELERTNQSGLERCEGEQDKRLHCYASWRNS |
| | ActRIIB-ECD | SGTIELVKKGCWLDDINCYDRQECVATEENPQVYFCCCEGNFC |
| | (AG-0024) | NERFTHLPEAGGPEVTYEPPPTAPT |
| 27 | Hybrid hu- | ETRECIYYNANWELERTNQSGLERCEGEQDKRLHCYASWRNS |
| | ActRIIB-ECD | SGTIELVKKGCWLDDINCYDRQECVATKENPQVYFCCCEGNF |
| | (AG-0025) | CNERFTHLPEAGGPEVTYEPPPTAPT |
| 28 | Hybrid hu- | ETRECIYYNANWELERTNQSGLERCEGDKDKRLHCYASWRNS |
| | ActRIIB-ECD | SGTIELVKKGCWLDDINCYDRQECVATKENPQVYFCCCEGNF |
| | (AG-0026) | CNERFTHLPEAGGPEVTYEPPPTAPT |
| 29 | Hybrid hu- | ETRECIYYNANWELERTNQSGLERCEGDQDKRLHCYASWRNS |
| | ActRIIB-ECD | SGTIELVKKGCWLDDINCYDRQECVATKENPQVYFCCCEGNF |
| | (AG-0027) | CNERFTHLPEAGGPEVTYEPPPTAPT |
| 30 | Hybrid hu- | ETRECIYYNANWELERTNQSGLERCEGDQDKRLHCYASWRNS |
| | ActRIIB-ECD | SGTIELVKKGCWLDDFNCYDRQECVATKENPQVYFCCCEGNF |
| | (AG-0028) | CNERFTHLPEAGGPEVTYEPPPTAPT |
| 31 | Hybrid hu- | ETRECIYYNANWELERTNQSGLERCEGDQDKRLHCYASWRNS |
| | ActRIIB-ECD | SGTIELVKKGCWLDDFNCYDRQECVATEENPQVYFCCCEGNF |
| | (AG-0029) | CNERFTHLPEAGGPEVTYEPPPTAPT |
| 32 | Hybrid hu- | ETRECIYYNANWELERTNQSGLERCYGDKDKRRHCYASWRNS |
| | ActRIIB-ECD | SGTIELVKKGCWLDDINCYDRQECVEKKDSPEVYFCCCEGNFC |
| | (AG-0030) | NERFTHLPEAGGPEVTYEPPPTAPT |
| 33 | Hybrid hu- | ETRECIFFNANWEKDRTNQTGVEPCEGEQDKRLHCYASWRNS |
| | ActRIIB-ECD | SGTIELVKKGCWLDDFNCYDRQECVATEENPQVYFCCCEGNF |
| | (AG-0031) | CNERFTHLPEAGGPEVTYEPPPTAPT |
| 34 | Hybrid hu- | ETRECIYYNANWELERTNQSGLERCEGEQDKRLHCFATWKNIS |
| | ActRIIB-ECD | GSIELVKQGCWLDDFNCYDRQECVATEENPQVYFCCCEGNFC |
| | (AG-0032) | NERFTHLPEAGGPEVTYEPPPTAPT |
| 35 | Hybrid hu- | ETRECIYYNANWELERTNQSGLERCEGEQDKRLHCYASWRNS |
| | ActRIIB-ECD | SGTIELVKKGCWLDDFNCYDRTDCVATEENPQVYFCCCEGNF |
| | (AG-0033) | CNERFTHLPEAGGPEVTYEPPPTAPT |
| 36 | Hybrid hu- | ETRECIYYNANWELERTNQSGLERCEGEQDKRLHCYASWRNS |
| | ActRIIB-ECD | SGTIELVKKGCWLDDFNCYDRQECVATEENPQVYFCCCEGNM |
| | (AG-0034) | CNERFTHLPEAGGPEVTYEPPPTAPT |
| 37 | Hybrid hu- | ETRECIYYNANWELERTNQSGLERCEGDQDKRLHCYASWRNS |
| | ActRIIB-ECD | SGTIELVKKGCWLDDINCYDRQECVATEENPQVYFCCCEGNFC |
| | (AG-0035) | NERFTHLPEAGGPEVTYEPPPTAPT |
| 51 | Hybrid hu- | ETQECLFFNANWEKDRTNQSGVEPCYGDKDKRRHCYASWRN |
| | ActRIIB-ECD | SSGTIELVKKGCWLDDFNCYDRQECVATEENPQVYFCCCEGN |
| | | FCNERFTHLPEAGGPEVTYEPPPTAPT |
| 52 | Hybrid hu- | ETQECLFFNANWEKDRINQSGVEPCEGEQDKRLHCYASWRNS |
| | ActRIIB-ECD | SGTIELVKKGCWLDDFNCYDRQECVATEENPQVYFCCCEGNF |
| | | CNERFTHLPEAGGPEVTYEPPPTAPT |
| 53 | Hybrid hu- | ETRECLFFNANWEKDRTNQSGVEPCEGEQDKRLHCYASWRNS |
| | ActRIIB-ECD | SGTIELVKKGCWLDDFNCYDRQECVATEENPQVYFCCCEGNF |
| | | CNERFTHLPEAGGPEVTYEPPPTAPT |
| 54 | Hybrid hu- | ETQECLFFNANWEKDRTNQSGVEPCYGEQDKRLHCYASWRNS |
| | ActRIIB-ECD | SGTIELVKKGCWLDDFNCYDRQECVATEENPQVYFCCCEGNF |
| | | CNERFTHLPEAGGPEVTYEPPPTAPT |
| 55 | Hybrid hu- | ETRECLFFNANWEKDRTNQSGVEPCYGDKDKRRHCYASWRN |
| | ActRIIB-ECD | SSGTIELVKKGCWLDDFNCYDRQECVATEENPQVYFCCCEGN |
| | | FCNERFTHLPEAGGPEVTYEPPPTAPT |
| 56 | Hybrid hu- | ETRECLFFNANWEKDRTNQTGVEPCYGDKDKRRHCFATWKNI |
| | ActRIIB-ECD | SGSIEIVKQGCWLDDFNCYDRQECVATEENPQVYFCCCEGNFC |
| | | NERFTHLPEAGGPEVTYEPPPTAPT |
| 57 | Hybrid hu- | ETRECLFFNANWEKDRTNQTGVEPCYGDKDKRRHCFATWKNI |
| | ActRIIB-ECD | SGSIEIVKQGCWLDDINCYDRQECVATEENPQVYFCCCEGNFC |
| | | NERFTHLPEAGGPEVTYEPPPTAPT |
| 58 | Hybrid hu- | ETRECLFFNANWEKDRTNQTGVEPCYGDKDKRRHCFATWKNI |
| | ActRIIB-ECD | SGSIEIVKQGCWLDDINCYDRTDCVEKKDSPEVYFCCCEGNFC |
| | | NERFTHLPEAGGPEVTYEPPPTAPT |
| 59 | Hybrid hu- | ETQECIYYNANWELERTNQSGLERCYGDKDKRRHCFATWKNI |
| | ActRIIB-ECD | SGSIEIVKQGCWLDDINCYDRQECVATEENPQVYFCCCEGNFC |
| | | NERFTHLPEAGGPEVTYEPPPTAPT |
| 60 | Hybrid hu- | ETRECIYYNANWELERTNQSGLERCYGDKDKRRHCFATWKNI |
| | ActRIIB-ECD | SGSIEIVKQGCWLDDINCYDRTDCVEKKDSPEVYFCCCEGNFC |
| | | NERFTHLPEAGGPEVTYEPPPTAPT |
| 61 | Hybrid hu- | ETRECIYYNANWELERTNQSGLERCYGDKDKRRHCFATWKNI |
| | ActRIIB-ECD | SGSIEIVKQGCWLDDINCYDRTDCVEKKDSPEVYFCCCEGNMC |
| | | NERFTHLPEAGGPEVTYEPPPTAPT |
| 62 | Hybrid hu- | ETRECIYYNANWELERTNQSGLERCEGEQDKRLHCFATWKNIS |
| | ActRIIB-ECD | GSIEIVKQGCWLDDINCYDRTDCVEKKDSPEVYFCCCEGNFCN |
| | | ERFTHLPEAGGPEVTYEPPPTAPT |
| 63 | Hybrid hu- | ETRECIYYNANWELERTNQSGLERCEGEQDKRLHCFATWKNIS |
| | ActRIIB-ECD | GSIEIVKQGCWLDDINCYDRTDCVATEENPQVYFCCCEGNFCN |
| | | ERFTHLPEAGGPEVTYEPPPTAPT |
| 64 | Hybrid hu- | ETRECIYYNANWELERTNQSGLERCEGEQDKRLHCFATWKNIS |
| | ActRIIB-ECD | GSIEIVKQGCWLDDFNCYDRTDCVATEENPQVYFCCCEGNFCN |
| | | ERFTHLPEAGGPEVTYEPPPTAPT |
| 65 | Hybrid hu- | ETRECIYYNANWELERTNQSGLERCEGEQDKRLHCFATWKNIS |
| | ActRIIB-ECD | GSIEIVKQGCWLDDFNCYDRQECVATEENPQVYFCCCEGNFCN |
| | | ERFTHLPEAGGPEVTYEPPPTAPT |
| 66 | Hybrid hu- | ETQECIYYNANWELERTNQSGLERCYGDKDKRRHCYASWRNS |
| | ActRIIB-ECD | SGTIELVKKGCWLDDINCYDRTDCVEKKDSPEVYFCCCEGNFC |
| | | NERFTHLPEAGGPEVTYEPPPTAPT |
| 67 | Hybrid hu- | ETQECIYYNANWELERTNQSGLERCYGDKDKRRHCYASWRNS |
| | ActRIIB-ECD | SGTIELVKKGCWLDDINCYDRTDCVEKKDSPEVYFCCCEGNM |
| | | CNERFTHLPEAGGPEVTYEPPPTAPT |
| 68 | Hybrid hu- | ETQECIYYNANWELERTNQSGLERCYGDKDKRRHCFATWKNI |
| | ActRIIB-ECD | SGSIEIVKQGCWLDDINCYDRTDCVEKKDSPEVYFCCCEGNMC |
| | | NERFTHLPEAGGPEVTYEPPPTAPT |
| 69 | Hybrid hu- | ETQECIYYNANWELERTNQSGLERCYGDKDKRRHCFATWKNI |
| | ActRIIB-ECD | SGSIEIVKQGCWLDDINCYDRTDCVEKKDSPEVYFCCCEGNFC |
| | | NERFTHLPEAGGPEVTYEPPPTAPT |
| 70 | Hybrid hu- | ETRECLFFNANWEKDRTNQTGVEPCEGEQDKRLHCFATWKNI |
| | ActRIIB-ECD | SGSIEIVKQGCWLDDINCYDRTDCVEKKDSPEVYFCCCEGNFC |
| | | NERFTHLPEAGGPEVTYEPPPTAPT |
| 71 | Hybrid hu- | ETRECLFFNANWEKDRTNQSGVEPCEGEQDKRLHCYASWRNS |
| | ActRIIB-ECD | SGTIELVKKGCWLDDINCYDRTDCVEKKDSPEVYFCCCEGNFC |
| | | NERFTHLPEAGGPEVTYEPPPTAPT |
| 72 | Hybrid hu- | ETRECLFFNANWEKDRTNQSGVEPCYGDKDKRRHCYASWRN |
| | ActRIIB-ECD | SSGTIELVKKGCWLDDINCYDRTDCVEKKDSPEVYFCCCEGNF |
| | | CNERFTHLPEAGGPEVTYEPPPTAPT |
| 73 | Hybrid hu- | ETRECIYYNANWELERTNQSGLERCYGDKDKRRHCYASWRNS |
| | ActRIIB-ECD | SGTIELVKKGCWLDDFNCYDRTDCVATEENPQVYFCCCEGNF |
| | | CNERFTHLPEAGGPEVTYEPPPTAPT |
| 74 | Hybrid hu- | ETRECIYYNANWELERTNQSGLERCYGDKDKRRHCYASWRNS |
| | ActRIIB-ECD | SGTIELVKQGCWLDDFNCYDRTDCVATEENPQVYFCCCEGNF |
| | | CNERFTHLPEAGGPEVTYEPPPTAPT |
| 75 | Hybrid hu- | ETRECIYYNANWELERTNQSGLERCYGDKDKRRHCYASWRNS |
| | ActRIIB-ECD | SGTIEIVKKGCWLDDFNCYDRTDCVATEENPQVYFCCCEGNFC |
| | | NERFTHLPEAGGPEVTYEPPPTAPT |
| 76 | Hybrid hu- | ETRECIYYNANWELERTNQSGLERCYGDKDKRRHCYASWRNS |
| | ActRIIB-ECD | SGSIELVKKGCWLDDFNCYDRTDCVATEENPQVYFCCCEGNF |
| | | CNERFTHLPEAGGPEVTYEPPPTAPT |
| 77 | Hybrid hu- | ETRECIYYNANWELERTNQSGLERCYGDKDKRRHCYASWRNS |
| | ActRIIB-ECD | SGSIEIVKKGCWLDDFNCYDRTDCVATEENPQVYFCCCEGNFC |
| | | NERFTHLPEAGGPEVTYEPPPTAPT |
| 78 | Hybrid hu- | ETRECIYYNANWELERTNQSGLERCYGDKDKRRHCYASWRNS |
| | ActRIIB-ECD | SGSIEIVKQGCWLDDFNCYDRTDCVATEENPQVYFCCCEGNFC |
| | | NERFTHLPEAGGPEVTYEPPPTAPT |
| 79 | Hybrid hu- | ETRECIYYNANWELERTNQSGLERCEGEQDKRLHCYASWRNS |
| | ActRIIB-ECD | SGTIELVKKGCWLDDFNCYDRTDCVATEENPQVYFCCCEGNF |
| | | CNERFTHLPEAGGPEVTYEPPPTAPT |
| 80 | Hybrid hu- | ETRECIYYNANWELERTNQSGLERCEGEQDKRLHCYASWRNS |
| | ActRIIB-ECD | SGTIELVKKGCWLDDFNCYDRQECVATEENPQVYFCCCEGNF |
| | | CNEKFSYFPEMEVTQPTSNPVTPKPP |
| 81 | Hybrid hu- | ETRECIYYNANWELERTNQSGLERCEGEQDKRLHCYASWRNS |
| | ActRIIB-ECD | SGTIELVKKGCWLDDFNCYDRQECVATEENPQVYFCCCEGNF |
| | | CNEKFSYFPQMEVTQPTSNPVTPKPP |
| 82 | Hybrid hu- | ETRECIYYNANWELERTNQSGLERCYGDKDKRRHCYASWRNS |
| | ActRIIB-ECD | SGTIELVKKGCWLDDINCYDRTDCVEKKDSPEVYFCCCEGNFC |
| | | NEKFSYFPEMEVTQPTSNPVTPKPP |
| 83 | Hybrid hu- | ETRECIYYNANWELERTNQSGLERCYGDKDKRRHCYASWRNS |
| | ActRIIB-ECD | SGTIELVKKGCWLDDINCYDRTDCVEKKDSPEVYFCCCEGNFC |
| | | NEKFSYFPQMEVTQPTSNPVTPKPP |
| 84 | Hybrid hu- | ETRECIYYNANWELERTNQSGLERCYGDKDKRRHCYASWRNS |
| | ActRIIB-ECD | SGTIELVKKGCWLDDFNCYDRQECVATEENPQVYFCCCEGNF |
| | | CNEKFSYFPQMEVTQPTSNPVTPKPP |
| 85 | Hybrid hu- | ETRECIYYNANWELERTNQSGLERCYGDKDKRRHCYASWRNS |
| | ActRIIB-ECD | SGTIELVKQGCWLDDFNCYDRQECVATEENPQVYFCCCEGNF |
| | | CNEKFSYFPQMEVTQPTSNPVTPKPP |
| 86 | Hybrid hu- | ETRECIYYNANWELERTNQSGLERCYGDKDKRRHCYASWRNS |
| | ActRIIB-ECD | SGTIEIVKKGCWLDDFNCYDRQECVATKENPQVYFCCCEGNFC |
| | | NEKFSYFPQMEVTQPTSNPVTPKPP |
| 87 | Hybrid hu- | ETRECIYYNANWELERTNQSGLERCYGDKDKRRHCYASWRNS |
| | ActRIIB-ECD | SGSIELVKKGCWLDDFNCYDRQECVATKENPQVYFCCCEGNF |
| | | CNEKFSYFPQMEVTQPTSNPVTPKPP |
| 88 | Hybrid hu- | ETRECIYYNANWELERTNQSGLERCEGEQDKRLHCYASWRNS |
| | ActRIIB-ECD | SGSIELVKKGCWLDDFNCYDRQECVATEENPQVYFCCCEGNF |
| | | CNEKFSYFPQMEVTQPTSNPVTPKPP |
| 89 | Hybrid hu- | ETRECIYYNANWELERTNQSGLERCEGEQDKRLHCYASWRNS |
| | ActRIIB-ECD | SGTIEIVKKGCWLDDFNCYDRQECVATEENPQVYFCCCEGNFC |
| | | NEKFSYFPQMEVTQPTSNPVTPKPP |
| 90 | Hybrid hu- | ETRECIYYNANWELERTNQSGLERCEGEQDKRLHCYASWRNS |
| | ActRIIB-ECD | SGTIELVKQGCWLDDFNCYDRQECVATEENPQVYFCCCEGNF |
| | | CNEKFSYFPQMEVTQPTSNPVTPKPP |
| 91 | Hybrid hu- | ETRECIYYNANWELERTNQSGLERCEGEQDKRLHCYASWRNS |
| | ActRIIB-ECD | SGTIELVKKGCWLDDFNCYDRQECVETEENPQVYFCCCEGNF |
| | | CNEKFSYFPQMEVTQPTSNPVTPKPP |
| 92 | Hybrid hu- | ETRECIYYNANWELERTNQSGLERCEGEQDKRLHCYASWRNS |
| | ActRIIB-ECD | SGTIELVKKGCWLDDFNCYDRQECVAKEENPQVYFCCCEGNF |
| | | CNEKFSYFPQMEVTQPTSNPVTPKPP |
| 93 | Hybrid hu- | ETRECIYYNANWELERTNQSGLERCEGEQDKRLHCYASWRNS |
| | ActRIIB-ECD | SGTIELVKKGCWLDDFNCYDRQECVATKENPQVYFCCCEGNF |
| | | CNEKFSYFPQMEVTQPTSNPVTPKPP |
| 94 | Hybrid hu- | ETRECIYYNANWELERTNQSGLERCEGEQDKRLHCYASWRNS |
| | ActRIIB-ECD | SGTIELVKKGCWLDDFNCYDRQECVATEDNPQVYFCCCEGNF |
| | | CNEKFSYFPQMEVTQPTSNPVTPKPP |
| 95 | Hybrid hu- | ETRECIYYNANWELERTNQSGLERCEGEQDKRLHCYASWRNS |
| | ActRIIB-ECD | SGTIELVKKGCWLDDFNCYDRQECVATEESPQVYFCCCEGNFC |
| | | NEKFSYFPQMEVTQPTSNPVTPKPP |
| 96 | Hybrid hu- | ETRECIYYNANWELERTNQSGLERCEGEQDKRLHCYASWRNS |
| | ActRIIB-ECD | SGTIELVKKGCWLDDFNCYDRQECVATEENPEVYFCCCEGNF |
| | | CNEKFSYFPQMEVTQPTSNPVTPKPP |
| 97 | Hybrid hu- | ETRECIYYNANWELERTNQSGLERCEGDKDKRLHCYASWRNS |
| | ActRIIB-ECD | SGTIELVKKGCWLDDFNCYDRQECVETEENPQVYFCCCEGNF |
| | | CNEKFSYFPQMEVTQPTSNPVTPKPP |
| 98 | Hybrid hu- | ETRECIYYNANWELERTNQSGLERCEGEKDKRLHCYASWRNS |
| | ActRIIB-ECD | SGTIELVKKGCWLDDFNCYDRQECVAKEENPQVYFCCCEGNF |
| | | CNEKFSYFPQMEVTQPTSNPVTPKPP |
| 99 | Hybrid hu- | ETRECIYYNANWELERTNQSGLERCEGDQDKRLHCYASWRNS |
| | ActRIIB-ECD | SGTIELVKKGCWLDDFNCYDRQECVATKENPQVYFCCCEGNF |
| | | CNEKFSYFPQMEVTQPTSNPVTPKPP |
| 100 | Hybrid hu- | ETRECIYYNANWELERTNQSGLERCEGDKDKRLHCYASWRNS |
| | ActRIIB-ECD | SGTIELVKQGCWLDDFNCYDRQECVAKKENPQVYFCCCEGNF |
| | | CNEKFSYFPQMEVTQPTSNPVTPKPP |
| 101 | Hybrid hu- | ETRECIYYNANWELERTNQSGLERCEGDKDKRLHCYASWRNS |
| | ActRIIB-ECD | SGTIEIVKQGCWLDDFNCYDRQECVAEKENPQVYFCCCEGNFC |
| | | NEKFSYFPQMEVTQPTSNPVTPKPP |
| 102 | Hybrid hu- | ETRECIYYNANWELERTNQSGLERCYGDQDKRLHCYASWRNS |
| | ActRIIB-ECD | SGSIEIVKQGCWLDDFNCYDRQECVAKKENPQVYFCCCEGNF |
| | | CNEKFSYFPQMEVTQPTSNPVTPKPP |
| 103 | Hybrid hu- | ETRECIYYNANWELERTNQSGLERCEGEKDKRRHCYASWRNS |
| | ActRIIB-ECD | SGTIEIVKKGCWLDDFNCYDRQECVATKENPQVYFCCCEGNFC |
| | | NEKFSYFPQMEVTQPTSNPVTPKPP |
| 104 | Hybrid hu- | ETRECIYYNANWELERTNQSGLERCYGDQDKRRHCYASWRNS |
| | ActRIIB-ECD | SGTIELVKKGCWLDDFNCYDRQECVATEENPEVYFCCCEGNF |
| | | CNEKFSYFPQMEVTQPTSNPVTPKPP |
| 105 | Hybrid hu- | ETRECIYYNANWELERTNQSGLERCEGEQDKRRHCYASWRNS |
| | ActRIIB-ECD | SGTIELVKKGCWLDDFNCYDRQECVATEENPQVYFCCCEGNF |
| | | CNEKFSYFPQMEVTQPTSNPVTPKPP |
| 106 | Hybrid hu- | ETRECIYYNANWELERTNQSGLERCYGEQDKRLHCYASWRNS |
| | ActRIIB-ECD | SGSIEIVKKGCWLDDFNCYDRTDCVATEENPQVYFCCCEGNFC |
| | | NEKFSYFPQMEVTQPTSNPVTPKPP |
| 107 | Hybrid hu- | ETRECIYYNANWELERTNQSGLERCEGEQDKRRHCYASWRNS |
| | ActRIIB-ECD | SGSIELVKKGCWLDDFNCYDRQECVAKEENPQVYFCCCEGNF |
| | | CNEKFSYFPQMEVTQPTSNPVTPKPP |
| 108 | Hybrid hu- | ETRECIYYNANWELERTNQSGLERCEGEQDKRRHCYASWRNS |
| | ActRIIB-ECD | SGTIEIVKKGCWLDDFNCYDRQECVAKEENPQVYFCCCEGNFC |
| | | NEKFSYFPQMEVTQPTSNPVTPKPP |
| 109 | Hybrid hu- | ETRECIYYNANWELERTNQSGLERCEGEQDKRRHCYASWRNS |
| | ActRIIB-ECD | SGSIEIVKKGCWLDDFNCYDRQECVATKENPQVYFCCCEGNFC |
| | | NEKFSYFPQMEVTQPTSNPVTPKPP |
| 110 | Hybrid hu- | ETRECIYYNANWELERTNQSGLERCEGDQDKRLHCYASWRNS |
| | ActRIIB-ECD | SGTIEIVKKGCWLDDFNCYDRQECVATKENPQVYFCCCEGNFC |
| | | NEKFSYFPQMEVTQPTSNPVTPKPP |
| 111 | Hybrid hu- | ETRECIYYNANWELERTNQSGLERCEGDQDKRLHCYASWRNS |
| | ActRIIB-ECD | SGSIELVKKGCWLDDFNCYDRQECVATKENPQVYFCCCEGNF |
| | | CNEKFSYFPQMEVTQPTSNPVTPKPP |
| 112 | Hybrid hu- | ETRECIYYNANWELERTNQSGLERCEGDQDKRLHCYASWRNS |
| | ActRIIB-ECD | SGTIELVKKGCWLDDFNCYDRQECVATKENPQVYFCCCEGNF |
| | | CNEKFSYFPQMEVTQPTSNPVTPKPP |
| 113 | Hybrid hu- | ETRECIYYNANWELERTNQSGLERCEGEQDKRLHCYASWRNS |
| | ActRIIB-ECD | SGTIEIVKKGCWLDDFNCYDRQECVATKENPQVYFCCCEGNFC |
| | | NEKFSYFPQMEVTQPTSNPVTPKPP |
| 114 | Hybrid hu- | ETRECIYYNANWELERTNQSGLERCEGEQDKRLHCYASWRNS |
| | ActRIIB-ECD | SGTIELVKKGCWLDDFNCYDRQECVATKENPQVYFCCCEGNF |
| | | CNEKFSYFPQMEVTQPTSNPVTPKPP |
| 115 | Hybrid hu- | ETRECIYYNANWELERTNQSGLERCEGDQDKRLHCYASWRNS |
| | ActRIIB-ECD | SGTIEIVKKGCWLDDFNCYDRQECVAKKENPQVYFCCCEGNF |
| | | CNEKFSYFPQMEVTQPTSNPVTPKPP |
| 116 | Hybrid hu- | ETQECLFFNANWEKDRTNQTGVEPCYGDKDKRRHCFATWKNI |
| | ActRIIB-ECD | SGSIEIVKQGCWLDDINCYDRTDCVEKKDSPEVYFCCCEGNMC |
| | | NERFTHLPEAGGPEVTYEPPPTAPT |
| 117 | Hybrid hu- | ETRECIYYNANWELERTNQSGLERCYGDKDKRRHCYASWRNS |
| | ActRIIB-ECD | SGTIELVKKGCWLDDINCYDRQECVATKENPQVYFCCCEGNF |
| | | CNERFTHLPEAGGPEVTYEPPPTAPT |
| 120 | N18Q mutant of | ETRECIYYNANWELERTQQSGLERCYGDKDKRRHCYASWRNS |
| | SEQ ID NO: 3 | SGTIELVKKGCWLDDINCYDRTDCVEKKDSPEVYFCCCEGNFC |
| | | NERFTHLPEAGGPEVTYEPPPTAPT |
| 121 | N18Q mutant of | ETRECIYYNANWELERTQQSGLERCYGDKDKRRHCYASWRNS |
| | SEQ ID NO: 4 | SGTIELVKKGCWLDDFNCYDRTDCVEKKDSPEVYFCCCEGNF |
| | | CNERFTHLPEAGGPEVTYEPPPTAPT |
| 122 | N18Q mutant of | ETRECIYYNANWELERTQQSGLERCEGEQDKRLHCYASWRNS |
| | SEQ ID NO: 5 | SGTIELVKKGCWLDDINCYDRTDCVEKKDSPEVYFCCCEGNFC |
| | | NERFTHLPEAGGPEVTYEPPPTAPT |
| 123 | N18Q mutant of | ETRECIYYNANWELERTQQSGLERCEGEQDKRLHCYASWRNS |
| | SEQ ID NO: 6 | SGTIELVKKGCWLDDINCYDRTDCVEKKDSPQVYFCCCEGNFC |
| | | NERFTHLPEAGGPEVTYEPPPTAPT |
| 124 | N18Q mutant of | ETRECIYYNANWELERTQQSGLERCEGEQDKRLHCYASWRNS |
| | SEQ ID NO: 7 | SGTIELVKKGCWLDDFNCYDRTDCVEKKDSPQVYFCCCEGNF |
| | | CNERFTHLPEAGGPEVTYEPPPTAPT |
| 125 | N18Q mutant of | ETRECIYYNANWELERTQQSGLERCEGEQDKRLHCYASWRNS |
| | SEQ ID NO: 8 | SGTIELVKKGCWLDDFNCYDRTDCVEKKDSPEVYFCCCEGNF |
| | | CNERFTHLPEAGGPEVTYEPPPTAPT |
| 126 | N18Q mutant of | ETRECIYYNANWELERTQQSGLERCEGEQDKRLHCYASWRNS |
| | SEQ ID NO: 9 | SGTIELVKKGCWLDDFNCYDRQECVEKKDSPEVYFCCCEGNF |
| | | CNERFTHLPEAGGPEVTYEPPPTAPT |
| 127 | N18Q mutant of | ETRECIYYNANWELERTQQSGLERCEGEQDKRLHCYASWRNS |
| | SEQ ID NO: 10 | SGTIELVKKGCWLDDFNCYDRQECVEKKDSPQVYFCCCEGNF |
| | | CNERFTHLPEAGGPEVTYEPPPTAPT |
| 128 | N18Q mutant of | TRECIYYNANWELERTQQSGLERCEGEQDKRLHCYASWRNSS |
| | SEQ ID NO: 11 | GTIELVKKGCWLDDINCYDRQECVEKKDSPEVYFCCCEGNFC |
| | | NERFTHLPEAGGPEVTYEPPPTAPT |
| 129 | N18Q mutant of | ETRECIYYNANWELERTQQSGLERCEGEQDKRLHCYASWRNS |
| | SEQ ID NO: 12 | SGTIELVKKGCWLDDFNCYDRTDCVEKKDSPEVYFCCCEGNM |
| | | CNERFTHLPEAGGPEVTYEPPPTAPT |
| 130 | N18Q mutant of | ETRECIYYNANWELERTQQSGLERCEGDQDKRLHCYASWRNS |
| | SEQ ID NO: 13 | SGTIELVKKGCWLDDFNCYDRQECVEKKDSPEVYFCCCEGNM |
| | | CNERFTHLPEAGGPEVTYEPPPTAPT |
| 131 | N18Q mutant of | ETQECIYYNANWEKDRTQQTGVEPCYGDKDKRRHCYASWRN |
| | SEQ ID NO: 14 | SSGTIELVKKGCWLDDFNCYDRQECVATEENPQVYFCCCEGN |
| | | FCNERFTHLPEAGGPEVTYEPPPTAPT |
| 132 | N18Q mutant of | ETQECIYYNANWEKDRTQQTGVEPCEGDQDKRLHCYASWRN |
| | SEQ ID NO: 15 | SSGTIELVKKGCWLDDFNCYDRQECVATEENPQVYFCCCEGN |
| | | FCNERFTHLPEAGGPEVTYEPPPTAPT |
| 133 | N18Q mutant of | ETRECIYYNANWELERTQQSGLERCYGDKDKRRHCYASWRNS |
| | SEQ ID NO: 16 | SGTIELVKKGCWLDDFNCYDRQECVATEENPQVYFCCCEGNF |
| | | CNERFTHLPEAGGPEVTYEPPPTAPT |
| 134 | N18Q mutant of | ETRECIYYNANWEKDRTQQTGVEPCEGDQDKRLHCYASWRN |
| | SEQ ID NO: 17 | SSGTIELVKKGCWLDDFNCYDRQECVATEENPQVYFCCCEGN |
| | | FCNERFTHLPEAGGPEVTYEPPPTAPT |
| 135 | N18Q mutant of | ETQECIYYNANWEKDRTQQTGVEPCEGDQDKRLHCYASWRN |
| | SEQ ID NO: 18 | SSGTIELVKKGCWLDDFNCYDRQECVATEENPQVYFCCCEGN |
| | | FCNERFTHLPEAGGPEVTYEPPPTAPT |
| 136 | N18Q mutant of | ETQECIYYNANWEKDRTQQTGLERCEGEQDKRLHCYASWRNS |
| | SEQ ID NO: | SGTIELVKKGCWLDDFNCYDRQECVATEENPQVYFCCCEGNF |
| | | CNERFTHLPEAGGPEVTYEPPPTAPT |
| 137 | N18Q mutant of | ETQECIYYNANWEKDRTQQSGLERCEGEQDKRLHCYASWRNS |
| | SEQ ID NO: 20 | SGTIELVKKGCWLDDFNCYDRQECVATEENPQVYFCCCEGNF |
| | | CNERFTHLPEAGGPEVTYEPPPTAPT |
| 138 | N18Q mutant of | ETRECIYYNANWEKDRTQQSGLERCEGEQDKRLHCYASWRNS |
| | SEQ ID NO: 21 | SGTIELVKKGCWLDDFNCYDRQECVATEENPQVYFCCCEGNF |
| | | CNERFTHLPEAGGPEVTYEPPPTAPT |
| 139 | N18Q mutant of | ETQECIYYNANWELERTQQSGLERCEGEQDKRLHCYASWRNS |
| | SEQ ID NO: 22 | SGTIELVKKGCWLDDFNCYDRQECVATEENPQVYFCCCEGNF |
| | | CNERFTHLPEAGGPEVTYEPPPTAPT |
| 140 | N18Q mutant of | ETRECIYYNANWELERTQQSGLERCEGEQDKRLHCFATWRNS |
| | SEQ ID NO: 23 | SGTIELVKQGCWLDDFNCYDRQECVATEENPQVYFCCCEGNF |
| | | CNERFTHLPEAGGPEVTYEPPPTAPT |
| 141 | N18Q mutant of | ETRECIYYNANWELERTQQSGLERCYGDKDKRRHCYASWRNS |
| | SEQ ID NO: 24 | SGTIELVKKGCWLDDINCYDRQECVATEENPQVYFCCCEGNFC |
| | | NERFTHLPEAGGPEVTYEPPPTAPT |
| 142 | N18Q mutant of | ETRECIYYNANWELERTQQSGLERCEGEQDKRLHCYASWRNS |
| | SEQ ID NO: 25 | SGTIELVKKGCWLDDFNCYDRQECVATKENPQVYFCCCEGNF |
| | | CNERFTHLPEAGGPEVTYEPPPTAPT |
| 143 | N18Q mutant of | ETRECIYYNANWELERTQQSGLERCEGEQDKRLHCYASWRNS |
| | SEQ ID NO: 26 | SGTIELVKKGCWLDDINCYDRQECVATEENPQVYFCCCEGNFC |
| | | NERFTHLPEAGGPEVTYEPPPTAPT |
| 144 | N18Q mutant of | ETRECIYYNANWELERTQQSGLERCEGEQDKRLHCYASWRNS |
| | SEQ ID NO: 27 | SGTIELVKKGCWLDDINCYDRQECVATKENPQVYFCCCEGNF |
| | | CNERFTHLPEAGGPEVTYEPPPTAPT |
| 145 | N18Q mutant of | ETRECIYYNANWELERTQQSGLERCEGDKDKRLHCYASWRNS |
| | SEQ ID NO: 28 | SGTIELVKKGCWLDDINCYDRQECVATKENPQVYFCCCEGNF |
| | | CNERFTHLPEAGGPEVTYEPPPTAPT |
| 146 | N18Q mutant of | ETRECIYYNANWELERTQQSGLERCEGDQDKRLHCYASWRNS |
| | SEQ ID NO: 29 | SGTIELVKKGCWLDDINCYDRQECVATKENPQVYFCCCEGNF |
| | | CNERFTHLPEAGGPEVTYEPPPTAPT |
| 147 | N18Q mutant of | ETRECIYYNANWELERTQQSGLERCEGDQDKRLHCYASWRNS |
| | SEQ ID NO: 30 | SGTIELVKKGCWLDDFNCYDRQECVATKENPQVYFCCCEGNF |
| | | CNERFTHLPEAGGPEVTYEPPPTAPT |
| 148 | N18Q mutant of | ETRECIYYNANWELERTQQSGLERCEGDQDKRLHCYASWRNS |
| | SEQ ID NO: 31 | SGTIELVKKGCWLDDFNCYDRQECVATEENPQVYFCCCEGNF |
| | | CNERFTHLPEAGGPEVTYEPPPTAPT |
| 149 | N18Q mutant of | ETRECIYYNANWELERTQQSGLERCYGDKDKRRHCYASWRNS |
| | SEQ ID NO: 32 | SGTIELVKKGCWLDDINCYDRQECVEKKDSPEVYFCCCEGNFC |
| | | NERFTHLPEAGGPEVTYEPPPTAPT |
| 150 | N18Q mutant of | ETRECIFFNANWEKDRTQQTGVEPCEGEQDKRLHCYASWRNS |
| | SEQ ID NO: 33 | SGTIELVKKGCWLDDFNCYDRQECVATEENPQVYFCCCEGNF |
| | | CNERFTHLPEAGGPEVTYEPPPTAPT |
| 151 | N18Q mutant of | ETRECIYYNANWELERTQQSGLERCEGEQDKRLHCFATWKNIS |
| | SEQ ID NO: 34 | GSIELVKQGCWLDDFNCYDRQECVATEENPQVYFCCCEGNFC |
| | | NERFTHLPEAGGPEVTYEPPPTAPT |
| 152 | N18Q mutant of | ETRECIYYNANWELERTQQSGLERCEGEQDKRLHCYASWRNS |
| | SEQ ID NO: 35 | SGTIELVKKGCWLDDFNCYDRTDCVATEENPQVYFCCCEGNF |
| | | CNERFTHLPEAGGPEVTYEPPPTAPT |
| 153 | N18Q mutant of | ETRECIYYNANWELERTQQSGLERCEGEQDKRLHCYASWRNS |
| | SEQ ID NO: 36 | SGTIELVKKGCWLDDFNCYDRQECVATEENPQVYFCCCEGNM |
| | | CNERFTHLPEAGGPEVTYEPPPTAPT |
| 154 | N18Q mutant of | ETRECIYYNANWELERTQQSGLERCEGDQDKRLHCYASWRNS |
| | SEQ ID NO: 37 | SGTIELVKKGCWLDDINCYDRQECVATEENPQVYFCCCEGNFC |
| | | NERFTHLPEAGGPEVTYEPPPTAPT |
| 155 | N18Q mutant of | ETQECLFFNANWEKDRTQQSGVEPCYGDKDKRRHCYASWRN |
| | SEQ ID NO: 51 | SSGTIELVKKGCWLDDFNCYDRQECVATEENPQVYFCCCEGN |
| | | FCNERFTHLPEAGGPEVTYEPPPTAPT |
| 156 | N18Q mutant of | ETQECLFFNANWEKDRTQQSGVEPCEGEQDKRLHCYASWRNS |
| | SEQ ID NO: 52 | SGTIELVKKGCWLDDFNCYDRQECVATEENPQVYFCCCEGNF |
| | | CNERFTHLPEAGGPEVTYEPPPTAPT |
| 157 | N18Q mutant of | ETRECLFFNANWEKDRTQQSGVEPCEGEQDKRLHCYASWRNS |
| | SEQ ID NO: 53 | SGTIELVKKGCWLDDFNCYDRQECVATEENPQVYFCCCEGNF |
| | | CNERFTHLPEAGGPEVTYEPPPTAPT |
| 158 | N18Q mutant of | ETQECLFFNANWEKDRTQQSGVEPCYGEQDKRLHCYASWRNS |
| | SEQ ID NO: 54 | SGTIELVKKGCWLDDFNCYDRQECVATEENPQVYFCCCEGNF |
| | | CNERFTHLPEAGGPEVTYEPPPTAPT |
| 159 | N18Q mutant of | ETRECLFFNANWEKDRTQQSGVEPCYGDKDKRRHCYASWRN |
| | SEQ ID NO: 55 | SSGTIELVKKGCWLDDFNCYDRQECVATEENPQVYFCCCEGN |
| | | FCNERFTHLPEAGGPEVTYEPPPTAPT |
| 160 | N18Q mutant of | ETRECLFFNANWEKDRTQQTGVEPCYGDKDKRRHCFATWKNI |
| | SEQ ID NO: 56 | SGSIEIVKQGCWLDDFNCYDRQECVATEENPQVYFCCCEGNFC |
| | | NERFTHLPEAGGPEVTYEPPPTAPT |
| 161 | N18Q mutant of | ETRECLFFNANWEKDRTQQTGVEPCYGDKDKRRHCFATWKNI |
| | SEQ ID NO: 57 | SGSIEIVKQGCWLDDINCYDRQECVATEENPQVYFCCCEGNFC |
| | | NERFTHLPEAGGPEVTYEPPPTAPT |
| 162 | N18Q mutant of | ETRECLFFNANWEKDRTQQTGVEPCYGDKDKRRHCFATWKNI |
| | SEQ ID NO: 58 | SGSIEIVKQGCWLDDINCYDRTDCVEKKDSPEVYFCCCEGNFC |
| | | NERFTHLPEAGGPEVTYEPPPTAPT |
| 163 | N18Q mutant of | ETQECIYYNANWELERTQQSGLERCYGDKDKRRHCFATWKNI |
| | SEQ ID NO: 59 | SGSIEIVKQGCWLDDINCYDRQECVATEENPQVYFCCCEGNFC |
| | | NERFTHLPEAGGPEVTYEPPPTAPT |
| 164 | N18Q mutant of | ETRECIYYNANWELERTQQSGLERCYGDKDKRRHCFATWKNI |
| | SEQ ID NO: 60 | SGSIEIVKQGCWLDDINCYDRTDCVEKKDSPEVYFCCCEGNFC |
| | | NERFTHLPEAGGPEVTYEPPPTAPT |
| 165 | N18Q mutant of | ETRECIYYNANWELERTQQSGLERCYGDKDKRRHCFATWKNI |
| | SEQ ID NO: 61 | SGSIEIVKQGCWLDDINCYDRTDCVEKKDSPEVYFCCCEGNMC |
| | | NERFTHLPEAGGPEVTYEPPPTAPT |
| 166 | N18Q mutant of | ETRECIYYNANWELERTQQSGLERCEGEQDKRLHCFATWKNIS |
| | SEQ ID NO: 62 | GSIEIVKQGCWLDDINCYDRTDCVEKKDSPEVYFCCCEGNFCN |
| | | ERFTHLPEAGGPEVTYEPPPTAPT |
| 167 | N18Q mutant of | ETRECIYYNANWELERTQQSGLERCEGEQDKRLHCFATWKNIS |
| | SEQ ID NO: 63 | GSIEIVKQGCWLDDINCYDRTDCVATEENPQVYFCCCEGNFCN |
| | | ERFTHLPEAGGPEVTYEPPPTAPT |
| 168 | N18Q mutant of | ETRECIYYNANWELERTQQSGLERCEGEQDKRLHCFATWKNIS |
| | SEQ ID NO: 64 | GSIEIVKQGCWLDDFNCYDRTDCVATEENPQVYFCCCEGNFCN |
| | | ERFTHLPEAGGPEVTYEPPPTAPT |
| 169 | N18Q mutant of | ETRECIYYNANWELERTQQSGLERCEGEQDKRLHCFATWKNIS |
| | SEQ ID NO: 65 | GSIEIVKQGCWLDDFNCYDRQECVATEENPQVYFCCCEGNFCN |
| | | ERFTHLPEAGGPEVTYEPPPTAPT |
| 170 | N18Q mutant of | ETQECIYYNANWELERTQQSGLERCYGDKDKRRHCYASWRNS |
| | SEQ ID NO: 66 | SGTIELVKKGCWLDDINCYDRTDCVEKKDSPEVYFCCCEGNFC |
| | | NERFTHLPEAGGPEVTYEPPPTAPT |
| 171 | N18Q mutant of | ETQECIYYNANWELERTQQSGLERCYGDKDKRRHCYASWRNS |
| | SEQ ID NO: 67 | SGTIELVKKGCWLDDINCYDRTDCVEKKDSPEVYFCCCEGNM |
| | | CNERFTHLPEAGGPEVTYEPPPTAPT |
| 172 | N18Q mutant of | ETQECIYYNANWELERTQQSGLERCYGDKDKRRHCFATWKNI |
| | SEQ ID NO: 68 | SGSIEIVKQGCWLDDINCYDRTDCVEKKDSPEVYFCCCEGNMC |
| | | NERFTHLPEAGGPEVTYEPPPTAPT |
| 173 | N18Q mutant of | ETQECIYYNANWELERTQQSGLERCYGDKDKRRHCFATWKNI |
| | SEQ ID NO: 69 | SGSIEIVKQGCWLDDINCYDRTDCVEKKDSPEVYFCCCEGNFC |
| | | NERFTHLPEAGGPEVTYEPPPTAPT |
| 174 | N18Q mutant of | ETRECLFFNANWEKDRTQQTGVEPCEGEQDKRLHCFATWKNI |
| | SEQ ID NO: 70 | SGSIEIVKQGCWLDDINCYDRTDCVEKKDSPEVYFCCCEGNFC |
| | | NERFTHLPEAGGPEVTYEPPPTAPT |
| 175 | N18Q mutant of | ETRECLFFNANWEKDRTQQSGVEPCEGEQDKRLHCYASWRNS |
| | SEQ ID NO: 71 | SGTIELVKKGCWLDDINCYDRTDCVEKKDSPEVYFCCCEGNFC |
| | | NERFTHLPEAGGPEVTYEPPPTAPT |
| 176 | N18Q mutant of | ETRECLFFNANWEKDRTQQSGVEPCYGDKDKRRHCYASWRN |
| | SEQ ID NO: 72 | SSGTIELVKKGCWLDDINCYDRTDCVEKKDSPEVYFCCCEGNF |
| | | CNERFTHLPEAGGPEVTYEPPPTAPT |
| 177 | N18Q mutant of | ETRECIYYNANWELERTQQSGLERCYGDKDKRRHCYASWRNS |
| | SEQ ID NO: 73 | SGTIELVKKGCWLDDFNCYDRTDCVATEENPQVYFCCCEGNF |
| | | CNERFTHLPEAGGPEVTYEPPPTAPT |
| 178 | N18Q mutant of | ETRECIYYNANWELERTQQSGLERCYGDKDKRRHCYASWRNS |
| | SEQ ID NO: 74 | SGTIELVKQGCWLDDFNCYDRTDCVATEENPQVYFCCCEGNF |
| | | CNERFTHLPEAGGPEVTYEPPPTAPT |
| 179 | N18Q mutant of | ETRECIYYNANWELERTQQSGLERCYGDKDKRRHCYASWRNS |
| | SEQ ID NO: 75 | SGTIEIVKKGCWLDDFNCYDRTDCVATEENPQVYFCCCEGNFC |
| | | NERFTHLPEAGGPEVTYEPPPTAPT |
| 180 | N18Q mutant of | ETRECIYYNANWELERTQQSGLERCYGDKDKRRHCYASWRNS |
| | SEQ ID NO: 76 | SGSIELVKKGCWLDDFNCYDRTDCVATEENPQVYFCCCEGNF |
| | | CNERFTHLPEAGGPEVTYEPPPTAPT |
| 181 | N18Q mutant of | ETRECIYYNANWELERTQQSGLERCYGDKDKRRHCYASWRNS |
| | SEQ ID NO: 77 | SGSIEIVKKGCWLDDFNCYDRTDCVATEENPQVYFCCCEGNFC |
| | | NERFTHLPEAGGPEVTYEPPPTAPT |
| 182 | N18Q mutant of | ETRECIYYNANWELERTQQSGLERCYGDKDKRRHCYASWRNS |
| | SEQ ID NO: 78 | SGSIEIVKQGCWLDDFNCYDRTDCVATEENPQVYFCCCEGNFC |
| | | NERFTHLPEAGGPEVTYEPPPTAPT |
| 183 | N18Q mutant of | ETRECIYYNANWELERTQQSGLERCEGEQDKRLHCYASWRNS |
| | SEQ ID NO: 79 | SGTIELVKKGCWLDDFNCYDRTDCVATEENPQVYFCCCEGNF |
| | | CNERFTHLPEAGGPEVTYEPPPTAPT |
| 184 | N18Q mutant of | ETRECIYYNANWELERTQQSGLERCEGEQDKRLHCYASWRNS |
| | SEQ ID NO: 80 | SGTIELVKKGCWLDDFNCYDRQECVATEENPQVYFCCCEGNF |
| | | CNEKFSYFPEMEVTQPTSNPVTPKPP |
| 185 | N18Q mutant of | ETRECIYYNANWELERTQQSGLERCEGEQDKRLHCYASWRNS |
| | SEQ ID NO: 81 | SGTIELVKKGCWLDDFNCYDRQECVATEENPQVYFCCCEGNF |
| | | CNEKFSYFPQMEVTQPTSNPVTPKPP |
| 186 | N18Q mutant of | ETRECIYYNANWELERTQQSGLERCYGDKDKRRHCYASWRNS |
| | SEQ ID NO: 82 | SGTIELVKKGCWLDDINCYDRTDCVEKKDSPEVYFCCCEGNFC |
| | | NEKFSYFPEMEVTQPTSNPVTPKPP |
| 187 | N18Q mutant of | ETRECIYYNANWELERTQQSGLERCYGDKDKRRHCYASWRNS |
| | SEQ ID NO: 83 | SGTIELVKKGCWLDDINCYDRTDCVEKKDSPEVYFCCCEGNFC |
| | | NEKFSYFPQMEVTQPTSNPVTPKPP |
| 188 | N18Q mutant of | ETRECIYYNANWELERTQQSGLERCYGDKDKRRHCYASWRNS |
| | SEQ ID NO: 84 | SGTIELVKKGCWLDDFNCYDRQECVATEENPQVYFCCCEGNF |
| | | CNEKFSYFPQMEVTQPTSNPVTPKPP |
| 189 | N18Q mutant of | ETRECIYYNANWELERTQQSGLERCYGDKDKRRHCYASWRNS |
| | SEQ ID NO: 85 | SGTIELVKQGCWLDDFNCYDRQECVATEENPQVYFCCCEGNF |
| | | CNEKFSYFPQMEVTQPTSNPVTPKPP |
| 190 | N18Q mutant of | ETRECIYYNANWELERTQQSGLERCYGDKDKRRHCYASWRNS |
| | SEQ ID NO: 86 | SGTIEIVKKGCWLDDFNCYDRQECVATKENPQVYFCCCEGNFC |
| | | NEKFSYFPQMEVTQPTSNPVTPKPP |
| 191 | N18Q mutant of | ETRECIYYNANWELERTQQSGLERCYGDKDKRRHCYASWRNS |
| | SEQ ID NO: 87 | SGSIELVKKGCWLDDFNCYDRQECVATKENPQVYFCCCEGNF |
| | | CNEKFSYFPQMEVTQPTSNPVTPKPP |
| 192 | N18Q mutant of | ETRECIYYNANWELERTQQSGLERCEGEQDKRLHCYASWRNS |
| | SEQ ID NO: 88 | SGSIELVKKGCWLDDFNCYDRQECVATEENPQVYFCCCEGNF |
| | | CNEKFSYFPQMEVTQPTSNPVTPKPP |
| 193 | N18Q mutant of | ETRECIYYNANWELERTQQSGLERCEGEQDKRLHCYASWRNS |
| | SEQ ID NO: 89 | SGTIEIVKKGCWLDDFNCYDRQECVATEENPQVYFCCCEGNFC |
| | | NEKFSYFPQMEVTQPTSNPVTPKPP |
| 194 | N18Q mutant of | ETRECIYYNANWELERTQQSGLERCEGEQDKRLHCYASWRNS |
| | SEQ ID NO: 90 | SGTIELVKQGCWLDDFNCYDRQECVATEENPQVYFCCCEGNF |
| | | CNEKFSYFPQMEVTQPTSNPVTPKPP |
| 195 | N18Q mutant of | ETRECIYYNANWELERTQQSGLERCEGEQDKRLHCYASWRNS |
| | SEQ ID NO: 91 | SGTIELVKKGCWLDDFNCYDRQECVETEENPQVYFCCCEGNF |
| | | CNEKFSYFPQMEVTQPTSNPVTPKPP |
| 196 | N18Q mutant of | ETRECIYYNANWELERTQQSGLERCEGEQDKRLHCYASWRNS |
| | SEQ ID NO: 92 | SGTIELVKKGCWLDDFNCYDRQECVAKEENPQVYFCCCEGNF |
| | | CNEKFSYFPQMEVTQPTSNPVTPKPP |
| 197 | N18Q mutant of | ETRECIYYNANWELERTQQSGLERCEGEQDKRLHCYASWRNS |
| | SEQ ID NO: 93 | SGTIELVKKGCWLDDFNCYDRQECVATKENPQVYFCCCEGNF |
| | | CNEKFSYFPQMEVTQPTSNPVTPKPP |
| 198 | N18Q mutant of | ETRECIYYNANWELERTQQSGLERCEGEQDKRLHCYASWRNS |
| | SEQ ID NO: 94 | SGTIELVKKGCWLDDFNCYDRQECVATEDNPQVYFCCCEGNF |
| | | CNEKFSYFPQMEVTQPTSNPVTPKPP |
| 199 | N18Q mutant of | ETRECIYYNANWELERTQQSGLERCEGEQDKRLHCYASWRNS |
| | SEQ ID NO: 95 | SGTIELVKKGCWLDDFNCYDRQECVATEESPQVYFCCCEGNFC |
| | | NEKFSYFPQMEVTQPTSNPVTPKPP |
| 200 | N18Q mutant of | ETRECIYYNANWELERTQQSGLERCEGEQDKRLHCYASWRNS |
| | SEQ ID NO: 96 | SGTIELVKKGCWLDDFNCYDRQECVATEENPEVYFCCCEGNF |
| | | CNEKFSYFPQMEVTQPTSNPVTPKPP |
| 201 | N18Q mutant of | ETRECIYYNANWELERTQQSGLERCEGDKDKRLHCYASWRNS |
| | SEQ ID NO: 97 | SGTIELVKKGCWLDDFNCYDRQECVETEENPQVYFCCCEGNF |
| | | CNEKFSYFPQMEVTQPTSNPVTPKPP |
| 202 | N18Q mutant of | ETRECIYYNANWELERTQQSGLERCEGEKDKRLHCYASWRNS |
| | SEQ ID NO: 98 | SGTIELVKKGCWLDDFNCYDRQECVAKEENPQVYFCCCEGNF |
| | | CNEKFSYFPQMEVTQPTSNPVTPKPP |
| 203 | N18Q mutant of | ETRECIYYNANWELERTQQSGLERCEGDQDKRLHCYASWRNS |
| | SEQ ID NO: 99 | SGTIELVKKGCWLDDFNCYDRQECVATKENPQVYFCCCEGNF |
| | | CNEKFSYFPQMEVTQPTSNPVTPKPP |
| 204 | N18Q mutant of | ETRECIYYNANWELERTQQSGLERCEGDKDKRLHCYASWRNS |
| | SEQ ID NO: 100 | SGTIELVKQGCWLDDFNCYDRQECVAKKENPQVYFCCCEGNF |
| | | CNEKFSYFPQMEVTQPTSNPVTPKPP |
| 205 | N18Q mutant of | ETRECIYYNANWELERTQQSGLERCEGDKDKRLHCYASWRNS |
| | SEQ ID NO: 101 | SGTIEIVKQGCWLDDFNCYDRQECVAEKENPQVYFCCCEGNFC |
| | | NEKFSYFPQMEVTQPTSNPVTPKPP |
| 206 | N18Q mutant of | ETRECIYYNANWELERTQQSGLERCYGDQDKRLHCYASWRNS |
| | SEQ ID NO: 102 | SGSIEIVKQGCWLDDFNCYDRQECVAKKENPQVYFCCCEGNF |
| | | CNEKFSYFPQMEVTQPTSNPVTPKPP |
| 207 | N18Q mutant of | ETRECIYYNANWELERTQQSGLERCEGEKDKRRHCYASWRNS |
| | SEQ ID NO: 103 | SGTIEIVKKGCWLDDFNCYDRQECVATKENPQVYFCCCEGNFC |
| | | NEKFSYFPQMEVTQPTSNPVTPKPP |
| 208 | N18Q mutant of | ETRECIYYNANWELERTQQSGLERCYGDQDKRRHCYASWRNS |
| | SEQ ID NO: 104 | SGTIELVKKGCWLDDFNCYDRQECVATEENPEVYFCCCEGNF |
| | | CNEKFSYFPQMEVTQPTSNPVTPKPP |
| 209 | N18Q mutant of | ETRECIYYNANWELERTQQSGLERCEGEQDKRRHCYASWRNS |
| | SEQ ID NO: 105 | SGTIELVKKGCWLDDFNCYDRQECVATEENPQVYFCCCEGNF |
| | | CNEKFSYFPQMEVTQPTSNPVTPKPP |
| 210 | N18Q mutant of | ETRECIYYNANWELERTQQSGLERCYGEQDKRLHCYASWRNS |
| | SEQ ID NO: 106 | SGSIEIVKKGCWLDDFNCYDRTDCVATEENPQVYFCCCEGNFC |
| | | NEKFSYFPQMEVTQPTSNPVTPKPP |
| 211 | N18Q mutant of | ETRECIYYNANWELERTQQSGLERCEGEQDKRRHCYASWRNS |
| | SEQ ID NO: 107 | SGSIELVKKGCWLDDFNCYDRQECVAKEENPQVYFCCCEGNF |
| | | CNEKFSYFPQMEVTQPTSNPVTPKPP |
| 212 | N18Q mutant of | ETRECIYYNANWELERTQQSGLERCEGEQDKRRHCYASWRNS |
| | SEQ ID NO: 108 | SGTIEIVKKGCWLDDFNCYDRQECVAKEENPQVYFCCCEGNFC |
| | | NEKFSYFPQMEVTQPTSNPVTPKPP |
| 213 | N18Q mutant of | ETRECIYYNANWELERTQQSGLERCEGEQDKRRHCYASWRNS |
| | SEQ ID NO: 109 | SGSIEIVKKGCWLDDFNCYDRQECVATKENPQVYFCCCEGNFC |
| | | NEKFSYFPQMEVTQPTSNPVTPKPP |
| 214 | N18Q mutant of | ETRECIYYNANWELERTQQSGLERCEGDQDKRLHCYASWRNS |
| | SEQ ID NO: 110 | SGTIEIVKKGCWLDDFNCYDRQECVATKENPQVYFCCCEGNFC |
| | | NEKFSYFPQMEVTQPTSNPVTPKPP |
| 215 | N18Q mutant of | ETRECIYYNANWELERTQQSGLERCEGDQDKRLHCYASWRNS |
| | SEQ ID NO: 111 | SGSIELVKKGCWLDDFNCYDRQECVATKENPQVYFCCCEGNF |
| | | CNEKFSYFPQMEVTQPTSNPVTPKPP |
| 216 | N18Q mutant of | ETRECIYYNANWELERTQQSGLERCEGDQDKRLHCYASWRNS |
| | SEQ ID NO: 112 | SGTIELVKKGCWLDDFNCYDRQECVATKENPQVYFCCCEGNF |
| | | CNEKFSYFPQMEVTQPTSNPVTPKPP |
| 217 | N18Q mutant of | ETRECIYYNANWELERTQQSGLERCEGEQDKRLHCYASWRNS |
| | SEQ ID NO: 113 | SGTIEIVKKGCWLDDFNCYDRQECVATKENPQVYFCCCEGNFC |
| | | NEKFSYFPQMEVTQPTSNPVTPKPP |
| 218 | N18Q mutant of | ETRECIYYNANWELERTQQSGLERCEGEQDKRLHCYASWRNS |
| | SEQ ID NO: 114 | SGTIELVKKGCWLDDFNCYDRQECVATKENPQVYFCCCEGNF |
| | | CNEKFSYFPQMEVTQPTSNPVTPKPP |
| 219 | N18Q mutant of | ETRECIYYNANWELERTQQSGLERCEGDQDKRLHCYASWRNS |
| | SEQ ID NO: 115 | SGTIEIVKKGCWLDDFNCYDRQECVAKKENPQVYFCCCEGNF |
| | | CNEKFSYFPQMEVTQPTSNPVTPKPP |
| 220 | N18Q mutant of | ETQECLFFNANWEKDRTQQTGVEPCYGDKDKRRHCFATWKNI |
| | SEQ ID NO: 116 | SGSIEIVKQGCWLDDINCYDRTDCVEKKDSPEVYFCCCEGNMC |
| | | NERFTHLPEAGGPEVTYEPPPTAPT |
| 221 | N18Q mutant of | ETRECIYYNANWELERTQQSGLERCYGDKDKRRHCYASWRNS |
| | SEQ ID NO: 117 | SGTIELVKKGCWLDDINCYDRQECVATKENPQVYFCCCEGNF |
| | | CNERFTHLPEAGGPEVTYEPPPTAPT |
| 38 | Human | ASTKGPSVFPLAPSSKSTSGGTAALGCLVKDYFPEPVTVSWNS |
| | immunoglobulin | GALTSGVHTFPAVLQSS |
| | gamma-1 heavy | GLYSLSSVVTVPSSSLGTQTYICNVNHKPSNTKVDKKVEPKSC |
| | chain constant | DKTHTCPPCPAPELLGG |
| | region | PSVFLFPPKPKDTLMISRTPEVTCVVVDVSHEDPEVKFNWYVD |
| | | GVEVHNAKTKPREEQYN |
| | | STYRVVSVLTVLHQDWLNGKEYKCKVSNKALPAPIEKTISKAK |
| | | GQPREPQVYTLPPSRDELTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPGK |
| 39 | IgG1 Fc Domain | VFLFPPKPKDTLMISRTPEVTCVVVDVSHEDPEVKFNWYVDGV |
| | | EVHNAKTKPREEQYNSTYRVVSVLTVLHQDWLNGKEYKCKV |
| | | SNKALPAPIEKTISKAKGQPREPQVYTLPPSRDELTKNQVSLTC |
| | | LVKGFYPSDIAVEWESNGQPENNYKTTPPVLDSDGSFFLYSKL |
| | | TVDKSRWQQGNVFSCSVMHEALHNHYTQKSLSLSPG |
| 40 | Human | ASTKGPSVFPLAPCSRSTSESTAALGCLVKDYFPEPVTVSWNSG |
| | immunoglobulin | ALTSGVHTFPAVLQSSGLYSLSSVVTVPSSNFGTQTYTCNVDH |
| | gamma-2 heavy | KPSNTKVDKTVERKCCVECPPCPAPPVAGPSVFLFPPKPKDTL |
| | chain constant | MISRTPEVTCVVVDVSHEDPEVQFNWYVDGVEVHNAKTKPRE |
| | region | EQFNSTFRVVSVLTVVHQDWLNGKEYKCKVSNKGLPAPIEKTI |
| | | SKTKGQPREPQVYTLPPSREEMTKNQVSLTCLVKGFYPSDIAV |
| | | EWESNGQPENNYKTTPPMLDSDGSFFLYSKLTVDKSRWQQGN |
| | | VFSCSVMHEALHNHYTQKSLSLSPGK |
| 41 | IgG2 Fc Domain | VFLFPPKPKDTLMISRTPEVTCVVVDVSHEDPEVQFNWYVDGV |
| | | EVHNAKTKPREEQFNSTFRVVSVLTVVHQDWLNGKEYKCKVS |
| | | NKGLPAPIEKTISKTKGQPREPQVYTLPPSREEMTKNQVSLTCL |
| | | VKGFYPSDIAVEWESNGQPENNYKTTPPMLDSDGSFFLYSKLT |
| | | VDKSRWQQGNVFCSVMHEALHNHYTQKSLSLSPG |
| 42 | Human | ASTKGPSVFPLAPCSRSTSESTAALGCLVKDYFPEPVTVSWNSG |
| | immunoglobulin | ALTSGVHTFPAVLQSSGLYSLSSVVTVPSSSLGTKTYTCNVDH |
| | gamma-4 heavy | KPSNTKVDKRVESKYGPPCPSCPAPEFLGGPSVFLFPPKPKDTL |
| | chain constant | MISRTPEVTCVVVDVSQEDPEVQFNWYVDGVEVHNAKTKPRE |
| | region | EQFNSTYRVVSVLTVLHQDWLNGKEYKCKVSNKGLPSSIEKTI |
| | | SKAKGQPREPQVYTLPPSQEEMTKNQVSLTCLVKGFYPSDIAV |
| | | EWESNGQPENNYKTTPPVLDSDGSFFLYSRLTVDKSRWQEGN |
| | | VFSCSVMHEALHNHYTQKSLSLSLGK |
| 43 | IgG4 Fc Domain | APEFLGGPSVFLFPPKPKDTLMISRTPEVTCVVVDVSQEDPEVQ |
| | | FNWYVDGVEVHNAKTKPREEQFNSTYRVVSVLTVLHQDWLN |
| | | GKEYKCKVSNKGLPSSIEKTISKAKGQPREPQVYTLPPSQEEMT |
| | | KNQVSLTCLVKGFYPSDIAVEWESNGQPENNYKTTPPVLDSDG |
| | | SFFLYSRLTVDKSRWQEGNVFSCSVMHEALHNHYTQKSLSLSL |
| | | G |
| 44 | Peptide Linker | GGGGS |
| | sequence | |
| 118 | Hinge linker | ESKYGPPCPPCP |
| 49 | ActRIIB native | MTAPWVALALLWGSLCAG |
| | signal peptide | |
| 50 | Immunoglobulin | MDMRVPAQLLGLLLLWLRGARC |
| | light chain signal | |
| | peptide | |
| 222 | Mutant soluble | ETRECIYYNANWELERTQQSGLERCEGDQDKRLHCYASWRNS |
| | ActRIIB-ECD | SGTIELVKKGCWLDDINCYDRQECVATKENPQVYFCCCEGNF |
| | N18Q fusion | CNERFTHLPEAGGPEVTYEPPPTAPTGGGGSESKYGPPCPPCPA |
| | protein | PEFLGGPSVFLFPPKPKDTLMISRTPEVTCVVVDVSQEDPEVQF |
| | | NWYVDGVEVHNAKTKPREEQFNSTYRVVSVLTVLHQDWLNG |
| | | KEYKCKVSNKGLPSSIEKTISKAKGQPREPQVYTLPPSQEEMTK |
| | | NQVSLTCLVKGFYPSDIAVEWESNGQPENNYKTTPPVLDSDGS |
| | | FFLYSRLTVDKSRWQEGNVFSCSVMHEALHNHYTQKSLSLSL |
| | | G |
| 224 | N18X mutant of | ETRECIYYNANWELERTXQSGLERCYGDKDKRRHCYASWRNS |
| | SEQ ID NO: 3 | SGTIELVKKGCWLDDINCYDRTDCVEKKDSPEVYFCCCEGNFC |
| | | NERFTHLPEAGGPEVTYEPPPTAPT |
| | | Wherein X is any amino acid that is not N |
| 225 | N18X mutant of | ETRECIYYNANWELERTXQSGLERCYGDKDKRRHCYASWRNS |
| | SEQ ID NO: 4 | SGTIELVKKGCWLDDFNCYDRTDCVEKKDSPEVYFCCCEGNF |
| | | CNERFTHLPEAGGPEVTYEPPPTAPT |
| | | Wherein X is any amino acid that is not N |
| 226 | N18X mutant of | ETRECIYYNANWELERTXQSGLERCEGEQDKRLHCYASWRNS |
| | SEQ ID NO: 5 | SGTIELVKKGCWLDDINCYDRTDCVEKKDSPEVYFCCCEGNFC |
| | | NERFTHLPEAGGPEVTYEPPPTAPT |
| | | Wherein X is any amino acid that is not N |
| 227 | N18X mutant of | ETRECIYYNANWELERTXQSGLERCEGEQDKRLHCYASWRNS |
| | SEQ ID NO: 6 | SGTIELVKKGCWLDDINCYDRTDCVEKKDSPQVYFCCCEGNFC |
| | | NERFTHLPEAGGPEVTYEPPPTAPT |
| | | Wherein X is any amino acid that is not N |
| 228 | N18X mutant of | ETRECIYYNANWELERTXQSGLERCEGEQDKRLHCYASWRNS |
| | SEQ ID NO: 7 | SGTIELVKKGCWLDDFNCYDRTDCVEKKDSPQVYFCCCEGNF |
| | | CNERFTHLPEAGGPEVTYEPPPTAPT |
| | | Wherein X is any amino acid that is not N |
| 229 | N18X mutant of | ETRECIYYNANWELERTXQSGLERCEGEQDKRLHCYASWRNS |
| | SEQ ID NO: 8 | SGTIELVKKGCWLDDFNCYDRTDCVEKKDSPEVYFCCCEGNF |
| | | CNERFTHLPEAGGPEVTYEPPPTAPT |
| | | Wherein X is any amino acid that is not N |
| 230 | N18X mutant of | ETRECIYYNANWELERTXQSGLERCEGEQDKRLHCYASWRNS |
| | SEQ ID NO: 9 | SGTIELVKKGCWLDDFNCYDRQECVEKKDSPEVYFCCCEGNF |
| | | CNERFTHLPEAGGPEVTYEPPPTAPT |
| | | Wherein X is any amino acid that is not N |
| 231 | N18X mutant of | ETRECIYYNANWELERTXQSGLERCEGEQDKRLHCYASWRNS |
| | SEQ ID NO: 10 | SGTIELVKKGCWLDDFNCYDRQECVEKKDSPQVYFCCCEGNF |
| | | CNERFTHLPEAGGPEVTYEPPPTAPT |
| | | Wherein X is any amino acid that is not N |
| 232 | N18X mutant of | TRECIYYNANWELERTXQSGLERCEGEQDKRLHCYASWRNSS |
| | SEQ ID NO: 11 | GTIELVKKGCWLDDINCYDRQECVEKKDSPEVYFCCCEGNFC |
| | | NERFTHLPEAGGPEVTYEPPPTAPT |
| | | Wherein X is any amino acid that is not N |
| 233 | N18X mutant of | ETRECIYYNANWELERTXQSGLERCEGEQDKRLHCYASWRNS |
| | SEQ ID NO: 12 | SGTIELVKKGCWLDDFNCYDRTDCVEKKDSPEVYFCCCEGNM |
| | | CNERFTHLPEAGGPEVTYEPPPTAPT |
| | | Wherein X is any amino acid that is not N |
| 234 | N18X mutant of | ETRECIYYNANWELERTXQSGLERCEGDQDKRLHCYASWRNS |
| | SEQ ID NO: 13 | SGTIELVKKGCWLDDFNCYDRQECVEKKDSPEVYFCCCEGNM |
| | | CNERFTHLPEAGGPEVTYEPPPTAPT |
| | | Wherein X is any amino acid that is not N |
| 235 | N18X mutant of | ETQECIYYNANWEKDRTXQTGVEPCYGDKDKRRHCYASWRN |
| | SEQ ID NO: 14 | SSGTIELVKKGCWLDDENCYDRQECVATEENPQVYFCCCEGN |
| | | FCNERFTHLPEAGGPEVTYEPPPTAPT |
| | | Wherein X is any amino acid that is not N |
| 236 | N18X mutant of | ETQECIYYNANWEKDRTXQTGVEPCEGDQDKRLHCYASWRN |
| | SEQ ID NO: 15 | SSGTIELVKKGCWLDDFNCYDRQECVATEENPQVYFCCCEGN |
| | | FCNERFTHLPEAGGPEVTYEPPPTAPT |
| | | Wherein X is any amino acid that is not N |
| 237 | N18X mutant of | ETRECIYYNANWELERTXQSGLERCYGDKDKRRHCYASWRNS |
| | SEQ ID NO: 16 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 238 | N18X mutant of | ETRECIYYNANWEKDRTXQTGVEPCEGDQDKRLHCYASWRN |
| | SEQ ID NO: 17 | SSGTIELVKKGCWLDDFNCYDRQECVATEENPQVYFCCCEGN |
| | | FCNERFTHLPEAGGPEVTYEPPPTAPT |
| | | Wherein X is any amino acid that is not N |
| 239 | N18X mutant of | ETQECIYYNANWEKDRTXQTGVEPCEGDQDKRLHCYASWRN |
| | SEQ ID NO: 18 | SSGTIELVKKGCWLDDFNCYDRQECVATEENPQVYFCCCEGN |
| | | FCNERFTHLPEAGGPEVTYEPPPTAPT |
| | | Wherein X is any amino acid that is not N |
| 240 | N18X mutant of | ETQECIYYNANWEKDRTXQTGLERCEGEQDKRLHCYASWRNS |
| | SEQ ID NO: | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 241 | N18X mutant of | ETQECIYYNANWEKDRTXQSGLERCEGEQDKRLHCYASWRNS |
| | SEQ ID NO: 20 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 242 | N18X mutant of | ETRECIYYNANWEKDRTXQSGLERCEGEQDKRLHCYASWRNS |
| | SEQ ID NO: 21 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 243 | N18X mutant of | ETQECIYYNANWELERTXQSGLERCEGEQDKRLHCYASWRNS |
| | SEQ ID NO: 22 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 244 | N18X mutant of | ETRECIYYNANWELERTXQSGLERCEGEQDKRLHCFATWRNS |
| | SEQ ID NO: 23 | SGTIELVKQGCWLDDFNCYDRQECVATEENPQVYFCCCEGNF |
| | | CNERFTHLPEAGGPEVTYEPPPTAPT |
| | | Wherein X is any amino acid that is not N |
| 245 | N18X mutant of | ETRECIYYNANWELERTXQSGLERCYGDKDKRRHCYASWRNS |
| | SEQ ID NO: 24 | SGTIELVKKGCWLDDINCYDRQECVATEENPQVYFCCCEGNFC |
| | | NERFTHLPEAGGPEVTYEPPPTAPT |
| | | Wherein X is any amino acid that is not N |
| 246 | N18X mutant of | ETRECIYYNANWELERTXQSGLERCEGEQDKRLHCYASWRNS |
| | SEQ ID NO: 25 | SGTIELVKKGCWLDDFNCYDRQECVATKENPQVYFCCCEGNF |
| | | CNERFTHLPEAGGPEVTYEPPPTAPT |
| | | Wherein X is any amino acid that is not N |
| 247 | N18X mutant of | ETRECIYYNANWELERTXQSGLERCEGEQDKRLHCYASWRNS |
| | SEQ ID NO: 26 | SGTIELVKKGCWLDDINCYDRQECVATEENPQVYFCCCEGNFC |
| | | NERFTHLPEAGGPEVTYEPPPTAPT |
| | | Wherein X is any amino acid that is not N |
| 248 | N18X mutant of | ETRECIYYNANWELERTXQSGLERCEGEQDKRLHCYASWRNS |
| | SEQ ID NO: 27 | SGTIELVKKGCWLDDINCYDRQECVATKENPQVYFCCCEGNF |
| | | CNERFTHLPEAGGPEVTYEPPPTAPT |
| | | Wherein X is any amino acid that is not N |
| 249 | N18X mutant of | ETRECIYYNANWELERTXQSGLERCEGDKDKRLHCYASWRNS |
| | SEQ ID NO: 28 | SGTIELVKKGCWLDDINCYDRQECVATKENPQVYFCCCEGNF |
| | | CNERFTHLPEAGGPEVTYEPPPTAPT |
| | | Wherein X is any amino acid that is not N |
| 250 | N18X mutant of | ETRECIYYNANWELERTXQSGLERCEGDQDKRLHCYASWRNS |
| | SEQ ID NO: 29 | SGTIELVKKGCWLDDINCYDRQECVATKENPQVYFCCCEGNF |
| | | CNERFTHLPEAGGPEVTYEPPPTAPT |
| | | Wherein X is any amino acid that is not N |
| 251 | N18X mutant of | ETRECIYYNANWELERTXQSGLERCEGDQDKRLHCYASWRNS |
| | SEQ ID NO: 30 | SGTIELVKKGCWLDDFNCYDRQECVATKENPQVYFCCCEGNF |
| | | CNERFTHLPEAGGPEVTYEPPPTAPT |
| | | Wherein X is any amino acid that is not N |
| 252 | N18X mutant of | ETRECIYYNANWELERTXQSGLERCEGDQDKRLHCYASWRNS |
| | SEQ ID NO: 31 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 253 | N18X mutant of | ETRECIYYNANWELERTXQSGLERCYGDKDKRRHCYASWRNS |
| | SEQ ID NO: 32 | SGTIELVKKGCWLDDINCYDRQECVEKKDSPEVYFCCCEGNFC |
| | | NERFTHLPEAGGPEVTYEPPPTAPT |
| | | Wherein X is any amino acid that is not N |
| 254 | N18X mutant of | ETRECIFFNANWEKDRTXQTGVEPCEGEQDKRLHCYASWRNS |
| | SEQ ID NO: 33 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 255 | N18X mutant of | ETRECIYYNANWELERTXQSGLERCEGEQDKRLHCFATWKNIS |
| | SEQ ID NO: 34 | GSIELVKQGCWLDDFNCYDRQECVATEENPQVYFCCCEGNFC |
| | | NERFTHLPEAGGPEVTYEPPPTAPT |
| | | Wherein X is any amino acid that is not N |
| 256 | N18X mutant of | ETRECIYYNANWELERTXQSGLERCEGEQDKRLHCYASWRNS |
| | SEQ ID NO: 35 | SGTIELVKKGCWLDDFNCYDRTDCVATEENPQVYFCCCEGNF |
| | | CNERFTHLPEAGGPEVTYEPPPTAPT |
| | | Wherein X is any amino acid that is not N |
| 257 | N18X mutant of | ETRECIYYNANWELERTXQSGLERCEGEQDKRLHCYASWRNS |
| | SEQ ID NO: 36 | SGTIELVKKGCWLDDFNCYDRQECVATEENPQVYFCCCEGNM |
| | | CNERFTHLPEAGGPEVTYEPPPTAPT |
| | | Wherein X is any amino acid that is not N |
| 258 | N18X mutant of | ETRECIYYNANWELERTXQSGLERCEGDQDKRLHCYASWRNS |
| | SEQ ID NO: 37 | SGTIELVKKGCWLDDINCYDRQECVATEENPQVYFCCCEGNFC |
| | | NERFTHLPEAGGPEVTYEPPPTAPT |
| | | Wherein X is any amino acid that is not N |
| 259 | N18X mutant of | ETQECLFFNANWEKDRTXQSGVEPCYGDKDKRRHCYASWRN |
| | SEQ ID NO: 51 | SSGTIELVKKGCWLDDFNCYDRQECVATEENPQVYFCCCEGN |
| | | FCNERFTHLPEAGGPEVTYEPPPTAPT |
| | | Wherein X is any amino acid that is not N |
| 260 | N18X mutant of | ETQECLFFNANWEKDRTXQSGVEPCEGEQDKRLHCYASWRNS |
| | SEQ ID NO: 52 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 261 | N18X mutant of | ETRECLFFNANWEKDRTXQSGVEPCEGEQDKRLHCYASWRNS |
| | SEQ ID NO: 53 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 262 | N18X mutant of | ETQECLFFNANWEKDRTXQSGVEPCYGEQDKRLHCYASWRNS |
| | SEQ ID NO: 54 | SGTIELVKKGCWLDDFNCYDRQECVATEENPQVYFCCCEGNF |
| | | CNERFTHLPEAGGPEVTYEPPPTAPT |
| | | Wherein X is any amino acid that is not N |
| 263 | N18X mutant of | ETRECLFFNANWEKDRTXQSGVEPCYGDKDKRRHCYASWRN |
| | SEQ ID NO: 55 | SSGTIELVKKGCWLDDFNCYDRQECVATEENPQVYFCCCEGN |
| | | FCNERFTHLPEAGGPEVTYEPPPTAPT |
| | | Wherein X is any amino acid that is not N |
| 264 | N18X mutant of | ETRECLFFNANWEKDRTXQTGVEPCYGDKDKRRHCFATWKNI |
| | SEQ ID NO: 56 | SGSIEIVKQGCWLDDFNCYDRQECVATEENPQVYFCCCEGNFC |
| | | NERFTHLPEAGGPEVTYEPPPTAPT |
| | | Wherein X is any amino acid that is not N |
| 265 | N18X mutant of | ETRECLFFNANWEKDRTXQTGVEPCYGDKDKRRHCFATWKNI |
| | SEQ ID NO: 57 | SGSIEIVKQGCWLDDINCYDRQECVATEENPQVYFCCCEGNFC |
| | | NERFTHLPEAGGPEVTYEPPPTAPT |
| | | Wherein X is any amino acid that is not N |
| 266 | N18X mutant of | ETRECLFFNANWEKDRTXQTGVEPCYGDKDKRRHCFATWKNI |
| | SEQ ID NO: 58 | SGSIEIVKQGCWLDDINCYDRTDCVEKKDSPEVYFCCCEGNFC |
| | | NERFTHLPEAGGPEVTYEPPPTAPT |
| | | Wherein X is any amino acid that is not N |
| 267 | N18X mutant of | ETQECIYYNANWELERTXQSGLERCYGDKDKRRHCFATWKNI |
| | SEQ ID NO: 59 | SGSIEIVKQGCWLDDINCYDRQECVATEENPQVYFCCCEGNFC |
| | | NERFTHLPEAGGPEVTYEPPPTAPT |
| | | Wherein X is any amino acid that is not N |
| 268 | N18X mutant of | ETRECIYYNANWELERTXQSGLERCYGDKDKRRHCFATWKNI |
| | SEQ ID NO: 60 | SGSIEIVKQGCWLDDINCYDRTDCVEKKDSPEVYFCCCEGNFC |
| | | NERFTHLPEAGGPEVTYEPPPTAPT |
| | | Wherein X is any amino acid that is not N |
| 269 | N18X mutant of | ETRECIYYNANWELERTXQSGLERCYGDKDKRRHCFATWKNI |
| | SEQ ID NO: 61 | SGSIEIVKQGCWLDDINCYDRTDCVEKKDSPEVYFCCCEGNMC |
| | | NERFTHLPEAGGPEVTYEPPPTAPT |
| | | Wherein X is any amino acid that is not N |
| 270 | N18X mutant of | ETRECIYYNANWELERTXQSGLERCEGEQDKRLHCFATWKNIS |
| | SEQ ID NO: 62 | GSIEIVKQGCWLDDINCYDRTDCVEKKDSPEVYFCCCEGNFCN |
| | | ERFTHLPEAGGPEVTYEPPPTAPT |
| | | Wherein X is any amino acid that is not N |
| 271 | N18X mutant of | ETRECIYYNANWELERTXQSGLERCEGEQDKRLHCFATWKNIS |
| | SEQ ID NO: 63 | GSIEIVKQGCWLDDINCYDRTDCVATEENPQVYFCCCEGNFCN |
| | | ERFTHLPEAGGPEVTYEPPPTAPT |
| | | Wherein X is any amino acid that is not N |
| 272 | N18X mutant of | ETRECIYYNANWELERTXQSGLERCEGEQDKRLHCFATWKNIS |
| | SEQ ID NO: 64 | GSIEIVKQGCWLDDFNCYDRTDCVATEENPQVYFCCCEGNFCN |
| | | ERFTHLPEAGGPEVTYEPPPTAPT |
| | | Wherein X is any amino acid that is not N |
| 273 | N18X mutant of | ETRECIYYNANWELERTXQSGLERCEGEQDKRLHCFATWKNIS |
| | SEQ ID NO: 65 | GSIEIVKQGCWLDDFNCYDRQECVATEENPQVYFCCCEGNFCN |
| | | ERFTHLPEAGGPEVTYEPPPTAPT |
| | | Wherein X is any amino acid that is not N |
| 274 | N18X mutant of | ETQECIYYNANWELERTXQSGLERCYGDKDKRRHCYASWRNS |
| | SEQ ID NO: 66 | SGTIELVKKGCWLDDINCYDRTDCVEKKDSPEVYFCCCEGNFC |
| | | NERFTHLPEAGGPEVTYEPPPTAPT |
| | | Wherein X is any amino acid that is not N |
| 275 | N18X mutant of | ETQECIYYNANWELERTXQSGLERCYGDKDKRRHCYASWRNS |
| | SEQ ID NO: 67 | SGTIELVKKGCWLDDINCYDRTDCVEKKDSPEVYFCCCEGNM |
| | | CNERFTHLPEAGGPEVTYEPPPTAPT |
| | | Wherein X is any amino acid that is not N |
| 276 | N18X mutant of | ETQECIYYNANWELERTXQSGLERCYGDKDKRRHCFATWKNI |
| | SEQ ID NO: 68 | SGSIEIVKQGCWLDDINCYDRTDCVEKKDSPEVYFCCCEGNMC |
| | | NERFTHLPEAGGPEVTYEPPPTAPT |
| | | Wherein X is any amino acid that is not N |
| 277 | N18X mutant of | ETQECIYYNANWELERTXQSGLERCYGDKDKRRHCFATWKNI |
| | SEQ ID NO: 69 | SGSIEIVKQGCWLDDINCYDRTDCVEKKDSPEVYFCCCEGNFC |
| | | NERFTHLPEAGGPEVTYEPPPTAPT |
| | | Wherein X is any amino acid that is not N |
| 278 | N18X mutant of | ETRECLFFNANWEKDRTXQTGVEPCEGEQDKRLHCFATWKNI |
| | SEQ ID NO: 70 | SGSIEIVKQGCWLDDINCYDRTDCVEKKDSPEVYFCCCEGNFC |
| | | NERFTHLPEAGGPEVTYEPPPTAPT |
| | | Wherein X is any amino acid that is not N |
| 279 | N18X mutant of | ETRECLFFNANWEKDRTXQSGVEPCEGEQDKRLHCYASWRNS |
| | SEQ ID NO: 71 | SGTIELVKKGCWLDDINCYDRTDCVEKKDSPEVYFCCCEGNFC |
| | | NERFTHLPEAGGPEVTYEPPPTAPT |
| | | Wherein X is any amino acid that is not N |
| 280 | N18X mutant of | ETRECLFFNANWEKDRTXQSGVEPCYGDKDKRRHCYASWRN |
| | SEQ ID NO: 72 | SSGTIELVKKGCWLDDINCYDRTDCVEKKDSPEVYFCCCEGNF |
| | | CNERFTHLPEAGGPEVTYEPPPTAPT |
| | | Wherein X is any amino acid that is not N |
| 281 | N18X mutant of | ETRECIYYNANWELERTXQSGLERCYGDKDKRRHCYASWRNS |
| | SEQ ID NO: 73 | SGTIELVKKGCWLDDFNCYDRTDCVATEENPQVYFCCCEGNF |
| | | CNERFTHLPEAGGPEVTYEPPPTAPT |
| | | Wherein X is any amino acid that is not N |
| 282 | N18X mutant of | ETRECIYYNANWELERTXQSGLERCYGDKDKRRHCYASWRNS |
| | SEQ ID NO: 74 | SGTIELVKQGCWLDDFNCYDRTDCVATEENPQVYFCCCEGNF |
| | | CNERFTHLPEAGGPEVTYEPPPTAPT |
| | | Wherein X is any amino acid that is not N |
| 283 | N18X mutant of | ETRECIYYNANWELERTXQSGLERCYGDKDKRRHCYASWRNS |
| | SEQ ID NO: 75 | SGTIEIVKKGCWLDDFNCYDRTDCVATEENPQVYFCCCEGNFC |
| | | NERFTHLPEAGGPEVTYEPPPTAPT |
| | | Wherein X is any amino acid that is not N |
| 284 | N18X mutant of | ETRECIYYNANWELERTXQSGLERCYGDKDKRRHCYASWRNS |
| | SEQ ID NO: 76 | SGSIELVKKGCWLDDFNCYDRTDCVATEENPQVYFCCCEGNF |
| | | CNERFTHLPEAGGPEVTYEPPPTAPT |
| | | Wherein X is any amino acid that is not N |
| 285 | N18X mutant of | ETRECIYYNANWELERTXQSGLERCYGDKDKRRHCYASWRNS |
| | SEQ ID NO: 77 | SGSIEIVKKGCWLDDFNCYDRTDCVATEENPQVYFCCCEGNFC |
| | | NERFTHLPEAGGPEVTYEPPPTAPT |
| | | Wherein X is any amino acid that is not N |
| 286 | N18X mutant of | ETRECIYYNANWELERTXQSGLERCYGDKDKRRHCYASWRNS |
| | SEQ ID NO: 78 | SGSIEIVKQGCWLDDFNCYDRTDCVATEENPQVYFCCCEGNFC |
| | | NERFTHLPEAGGPEVTYEPPPTAPT |
| | | Wherein X is any amino acid that is not N |
| 287 | N18X mutant of | ETRECIYYNANWELERTXQSGLERCEGEQDKRLHCYASWRNS |
| | SEQ ID NO: 79 | SGTIELVKKGCWLDDFNCYDRTDCVATEENPQVYFCCCEGNF |
| | | CNERFTHLPEAGGPEVTYEPPPTAPT |
| | | Wherein X is any amino acid that is not N |
| 288 | N18X mutant of | ETRECIYYNANWELERTXQSGLERCEGEQDKRLHCYASWRNS |
| | SEQ ID NO: 80 | SGTIELVKKGCWLDDFNCYDRQECVATEENPQVYFCCCEGNF |
| | | CNEKFSYFPEMEVTQPTSNPVTPKPP |
| | | Wherein X is any amino acid that is not N |
| 289 | N18X mutant of | ETRECIYYNANWELERTXQSGLERCEGEQDKRLHCYASWRNS |
| | SEQ ID NO: 81 | SGTIELVKKGCWLDDFNCYDRQECVATEENPQVYFCCCEGNF |
| | | CNEKFSYFPQMEVTQPTSNPVTPKPP |
| | | Wherein X is any amino acid that is not N |
| 290 | N18X mutant of | ETRECIYYNANWELERTXQSGLERCYGDKDKRRHCYASWRNS |
| | SEQ ID NO: 82 | SGTIELVKKGCWLDDINCYDRTDCVEKKDSPEVYFCCCEGNFC |
| | | NEKFSYFPEMEVTQPTSNPVTPKPP |
| | | Wherein X is any amino acid that is not N |
| 291 | N18X mutant of | ETRECIYYNANWELERTXQSGLERCYGDKDKRRHCYASWRNS |
| | SEQ ID NO: 83 | SGTIELVKKGCWLDDINCYDRTDCVEKKDSPEVYFCCCEGNFC |
| | | NEKFSYFPQMEVTQPTSNPVTPKPP |
| | | Wherein X is any amino acid that is not N |
| 292 | N18X mutant of | ETRECIYYNANWELERTXQSGLERCYGDKDKRRHCYASWRNS |
| | SEQ ID NO: 84 | SGTIELVKKGCWLDDFNCYDRQECVATEENPQVYFCCCEGNF |
| | | CNEKFSYFPQMEVTQPTSNPVTPKPP |
| | | Wherein X is any amino acid that is not N |
| 293 | N18X mutant of | ETRECIYYNANWELERTXQSGLERCYGDKDKRRHCYASWRNS |
| | SEQ ID NO: 85 | SGTIELVKQGCWLDDFNCYDRQECVATEENPQVYFCCCEGNF |
| | | CNEKFSYFPQMEVTQPTSNPVTPKPP |
| | | Wherein X is any amino acid that is not N |
| 294 | N18X mutant of | ETRECIYYNANWELERTXQSGLERCYGDKDKRRHCYASWRNS |
| | SEQ ID NO: 86 | SGTIEIVKKGCWLDDFNCYDRQECVATKENPQVYFCCCEGNFC |
| | | NEKFSYFPQMEVTQPTSNPVTPKPP |
| | | Wherein X is any amino acid that is not N |
| 295 | N18X mutant of | ETRECIYYNANWELERTXQSGLERCYGDKDKRRHCYASWRNS |
| | SEQ ID NO: 87 | SGSIELVKKGCWLDDFNCYDRQECVATKENPQVYFCCCEGNF |
| | | CNEKFSYFPQMEVTQPTSNPVTPKPP |
| | | Wherein X is any amino acid that is not N |
| 296 | N18X mutant of | ETRECIYYNANWELERTXQSGLERCEGEQDKRLHCYASWRNS |
| | SEQ ID NO: 88 | SGSIELVKKGCWLDDFNCYDRQECVATEENPQVYFCCCEGNF |
| | | CNEKFSYFPQMEVTQPTSNPVTPKPP |
| | | Wherein X is any amino acid that is not N |
| 297 | N18X mutant of | ETRECIYYNANWELERTXQSGLERCEGEQDKRLHCYASWRNS |
| | SEQ ID NO: 89 | SGTIEIVKKGCWLDDFNCYDRQECVATEENPQVYFCCCEGNFC |
| | | NEKFSYFPQMEVTQPTSNPVTPKPP |
| | | Wherein X is any amino acid that is not N |
| 298 | N18X mutant of | ETRECIYYNANWELERTXQSGLERCEGEQDKRLHCYASWRNS |
| | SEQ ID NO: 90 | SGTIELVKQGCWLDDFNCYDRQECVATEENPQVYFCCCEGNF |
| | | CNEKFSYFPQMEVTQPTSNPVTPKPP |
| | | Wherein X is any amino acid that is not N |
| 299 | N18X mutant of | ETRECIYYNANWELERTXQSGLERCEGEQDKRLHCYASWRNS |
| | SEQ ID NO: 91 | SGTIELVKKGCWLDDFNCYDRQECVETEENPQVYFCCCEGNF |
| | | CNEKFSYFPQMEVTQPTSNPVTPKPP |
| | | Wherein X is any amino acid that is not N |
| 300 | N18X mutant of | ETRECIYYNANWELERTXQSGLERCEGEQDKRLHCYASWRNS |
| | SEQ ID NO: 92 | SGTIELVKKGCWLDDFNCYDRQECVAKEENPQVYFCCCEGNF |
| | | CNEKFSYFPQMEVTQPTSNPVTPKPP |
| | | Wherein X is any amino acid that is not N |
| 301 | N18X mutant of | ETRECIYYNANWELERTXQSGLERCEGEQDKRLHCYASWRNS |
| | SEQ ID NO: 93 | SGTIELVKKGCWLDDFNCYDRQECVATKENPQVYFCCCEGNF |
| | | CNEKFSYFPQMEVTQPTSNPVTPKPP |
| | | Wherein X is any amino acid that is not N |
| 302 | N18X mutant of | ETRECIYYNANWELERTXQSGLERCEGEQDKRLHCYASWRNS |
| | SEQ ID NO: 94 | SGTIELVKKGCWLDDFNCYDRQECVATEDNPQVYFCCCEGNF |
| | | CNEKFSYFPQMEVTQPTSNPVTPKPP |
| | | Wherein X is any amino acid that is not N |
| 303 | N18X mutant of | ETRECIYYNANWELERTXQSGLERCEGEQDKRLHCYASWRNS |
| | SEQ ID NO: 95 | SGTIELVKKGCWLDDFNCYDRQECVATEESPQVYFCCCEGNFC |
| | | NEKFSYFPQMEVTQPTSNPVTPKPP |
| | | Wherein X is any amino acid that is not N |
| 304 | N18X mutant of | ETRECIYYNANWELERTXQSGLERCEGEQDKRLHCYASWRNS |
| | SEQ ID NO: 96 | SGTIELVKKGCWLDDFNCYDRQECVATEENPEVYFCCCEGNF |
| | | CNEKFSYFPQMEVTQPTSNPVTPKPP |
| | | Wherein X is any amino acid that is not N |
| 305 | N18X mutant of | ETRECIYYNANWELERTXQSGLERCEGDKDKRLHCYASWRNS |
| | SEQ ID NO: 97 | SGTIELVKKGCWLDDFNCYDRQECVETEENPQVYFCCCEGNF |
| | | CNEKFSYFPQMEVTQPTSNPVTPKPP |
| | | Wherein X is any amino acid that is not N |
| 306 | N18X mutant of | ETRECIYYNANWELERTXQSGLERCEGEKDKRLHCYASWRNS |
| | SEQ ID NO: 98 | SGTIELVKKGCWLDDFNCYDRQECVAKEENPQVYFCCCEGNF |
| | | CNEKFSYFPQMEVTQPTSNPVTPKPP |
| | | Wherein X is any amino acid that is not N |
| 307 | N18X mutant of | ETRECIYYNANWELERTXQSGLERCEGDQDKRLHCYASWRNS |
| | SEQ ID NO: 99 | SGTIELVKKGCWLDDFNCYDRQECVATKENPQVYFCCCEGNF |
| | | CNEKFSYFPQMEVTQPTSNPVTPKPP |
| | | Wherein X is any amino acid that is not N |
| 308 | N18X mutant of | ETRECIYYNANWELERTXQSGLERCEGDKDKRLHCYASWRNS |
| | SEQ ID NO: 100 | SGTIELVKQGCWLDDFNCYDRQECVAKKENPQVYFCCCEGNF |
| | | CNEKFSYFPQMEVTQPTSNPVTPKPP |
| | | Wherein X is any amino acid that is not N |
| 309 | N18X mutant of | ETRECIYYNANWELERTXQSGLERCEGDKDKRLHCYASWRNS |
| | | SGTIEIVKQGCWLDDFNCYDRQECVAEKENPQVYFCCCEGNFC |
| | | NEKFSYFPQMEVTQPTSNPVTPKPP |
| | SEQ ID NO: 101 | Wherein X is any amino acid that is not N |
| 310 | N18X mutant of | ETRECIYYNANWELERTXQSGLERCYGDQDKRLHCYASWRNS |
| | SEQ ID NO: 102 | SGSIEIVKQGCWLDDFNCYDRQECVAKKENPQVYFCCCEGNF |
| | | CNEKFSYFPQMEVTQPTSNPVTPKPP |
| | | Wherein X is any amino acid that is not N |
| 311 | N18X mutant of | ETRECIYYNANWELERTXQSGLERCEGEKDKRRHCYASWRNS |
| | SEQ ID NO: 103 | SGTIEIVKKGCWLDDFNCYDRQECVATKENPQVYFCCCEGNFC |
| | | NEKFSYFPQMEVTQPTSNPVTPKPP |
| | | Wherein X is any amino acid that is not N |
| 312 | N18X mutant of | ETRECIYYNANWELERTXQSGLERCYGDQDKRRHCYASWRNS |
| | SEQ ID NO: 104 | SGTIELVKKGCWLDDFNCYDRQECVATEENPEVYFCCCEGNF |
| | | CNEKFSYFPQMEVTQPTSNPVTPKPP |
| | | Wherein X is any amino acid that is not N |
| 313 | N18X mutant of | ETRECIYYNANWELERTXQSGLERCEGEQDKRRHCYASWRNS |
| | SEQ ID NO: 105 | SGTIELVKKGCWLDDFNCYDRQECVATEENPQVYFCCCEGNF |
| | | CNEKFSYFPQMEVTQPTSNPVTPKPP |
| | | Wherein X is any amino acid that is not N |
| 314 | N18X mutant of | ETRECIYYNANWELERTXQSGLERCYGEQDKRLHCYASWRNS |
| | SEQ ID NO: 106 | SGSIEIVKKGCWLDDFNCYDRTDCVATEENPQVYFCCCEGNFC |
| | | NEKFSYFPQMEVTQPTSNPVTPKPP |
| | | Wherein X is any amino acid that is not N |
| 315 | N18X mutant of | ETRECIYYNANWELERTXQSGLERCEGEQDKRRHCYASWRNS |
| | SEQ ID NO: 107 | SGSIELVKKGCWLDDFNCYDRQECVAKEENPQVYFCCCEGNF |
| | | CNEKFSYFPQMEVTQPTSNPVTPKPP |
| | | Wherein X is any amino acid that is not N |
| 316 | N18X mutant of | ETRECIYYNANWELERTXQSGLERCEGEQDKRRHCYASWRNS |
| | SEQ ID NO: 108 | SGTIEIVKKGCWLDDFNCYDRQECVAKEENPQVYFCCCEGNFC |
| | | NEKFSYFPQMEVTQPTSNPVTPKPP |
| | | Wherein X is any amino acid that is not N |
| 317 | N18X mutant of | ETRECIYYNANWELERTXQSGLERCEGEQDKRRHCYASWRNS |
| | SEQ ID NO: 109 | SGSIEIVKKGCWLDDFNCYDRQECVATKENPQVYFCCCEGNFC |
| | | NEKFSYFPQMEVTQPTSNPVTPKPP |
| | | Wherein X is any amino acid that is not N |
| 318 | N18X mutant of | ETRECIYYNANWELERTXQSGLERCEGDQDKRLHCYASWRNS |
| | SEQ ID NO: 110 | SGTIEIVKKGCWLDDFNCYDRQECVATKENPQVYFCCCEGNFC |
| | | NEKFSYFPQMEVTQPTSNPVTPKPP |
| | | Wherein X is any amino acid that is not N |
| 319 | N18X mutant of | ETRECIYYNANWELERTXQSGLERCEGDQDKRLHCYASWRNS |
| | SEQ ID NO: 111 | SGSIELVKKGCWLDDFNCYDRQECVATKENPQVYFCCCEGNF |
| | | CNEKFSYFPQMEVTQPTSNPVTPKPP |
| | | Wherein X is any amino acid that is not N |
| 320 | N18X mutant of | ETRECIYYNANWELERTXQSGLERCEGDQDKRLHCYASWRNS |
| | SEQ ID NO: 112 | SGTIELVKKGCWLDDFNCYDRQECVATKENPQVYFCCCEGNF |
| | | CNEKFSYFPQMEVTQPTSNPVTPKPP |
| | | Wherein X is any amino acid that is not N |
| 321 | N18X mutant of | ETRECIYYNANWELERTXQSGLERCEGEQDKRLHCYASWRNS |
| | SEQ ID NO: 113 | SGTIEIVKKGCWLDDFNCYDRQECVATKENPQVYFCCCEGNFC |
| | | NEKFSYFPQMEVTQPTSNPVTPKPP |
| | | Wherein X is any amino acid that is not N |
| 322 | N18X mutant of | ETRECIYYNANWELERTXQSGLERCEGEQDKRLHCYASWRNS |
| | SEQ ID NO: 114 | SGTIELVKKGCWLDDFNCYDRQECVATKENPQVYFCCCEGNF |
| | | CNEKFSYFPQMEVTQPTSNPVTPKPP |
| | | Wherein X is any amino acid that is not N |
| 323 | N18X mutant of | ETRECIYYNANWELERTXQSGLERCEGDQDKRLHCYASWRNS |
| | SEQ ID NO: 115 | SGTIEIVKKGCWLDDFNCYDRQECVAKKENPQVYFCCCEGNF |
| | | CNEKFSYFPQMEVTQPTSNPVTPKPP |
| | | Wherein X is any amino acid that is not N |
| 324 | N18X mutant of | ETQECLFFNANWEKDRTXQTGVEPCYGDKDKRRHCFATWKNI |
| | SEQ ID NO: 116 | SGSIEIVKQGCWLDDINCYDRTDCVEKKDSPEVYFCCCEGNMC |
| | | NERFTHLPEAGGPEVTYEPPPTAPT |
| | | Wherein X is any amino acid that is not N |
| 325 | N18X mutant of | ETRECIYYNANWELERTXQSGLERCYGDKDKRRHCYASWRNS |
| | SEQ ID NO: 117 | SGTIELVKKGCWLDDINCYDRQECVATKENPQVYFCCCEGNF |
| | | CNERFTHLPEAGGPEVTYEPPPTAPT |
| | | Wherein X is any amino acid that is not N |
| 326 | S20X mutant of | ETRECIYYNANWELERTNQXGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| | | Wherein X is any amino acid that is not S or T |
| 327 | L55D mutant of | ETRECIYYNANWELERTNQSGLERCEGEQDKRLHCYASWRNS |
| | SEQ ID NO: 1 | SGTIELVKKGCWDDDFNCYDRQECVATEENPQVYFCCCEGNF |
| | | CNERFTHLPEAGGPEVTYEPPPTAPT |
| 328 | N18X and L55D | ETRECIYYNANWELERTXQSGLERCEGEQDKRLHCYASWRNS |
| | mutant of SEQ ID | SGTIELVKKGCWDDDFNCYDRQECVATEENPQVYFCCCEGNF |
| | NO: 1 | CNERFTHLPEAGGPEVTYEPPPTAPT |
| | | Wherein X is any amino acid that is not N |
| 329 | N18Q and L55D | ETRECIYYNANWELERTQQSGLERCEGEQDKRLHCYASWRNS |
| | mutant of SEQ ID | SGTIELVKKGCWDDDFNCYDRQECVATEENPQVYFCCCEGNF |
| | NO: 1 | CNERFTHLPEAGGPEVTYEPPPTAPT |
| 330 | L55E mutant of | ETRECIYYNANWELERTNQSGLERCEGEQDKRLHCYASWRNS |
| | SEQ ID NO: 1 | SGTIELVKKGCWEDDFNCYDRQECVATEENPQVYFCCCEGNF |
| | | CNERFTHLPEAGGPEVTYEPPPTAPT |
| 331 | N18X and L55E | ETRECIYYNANWELERTXQSGLERCEGEQDKRLHCYASWRNS |
| | mutant of SEQ ID | SGTIELVKKGCWEDDFNCYDRQECVATEENPQVYFCCCEGNF |
| | NO: 1 | CNERFTHLPEAGGPEVTYEPPPTAPT |
| 332 | N18Q and L55E | ETRECIYYNANWELERTQQSGLERCEGEQDKRLHCYASWRNS |
| | mutant of SEQ ID | SGTIELVKKGCWEDDFNCYDRQECVATEENPQVYFCCCEGNF |
| | NO: 1 | CNERFTHLPEAGGPEVTYEPPPTAPT |
| 333 | R40A mutant of | ETRECIYYNANWELERTNQSGLERCEGEQDKRLHCYASWANS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 334 | N18X and R40A | ETRECIYYNANWELERTXQSGLERCEGEQDKRLHCYASWANS |
| | mutant of SEQ ID | SGTIELVKKGCWLDDFNCYDRQECVATEENPQVYFCCCEGNF |
| | NO: 1 | CNERFTHLPEAGGPEVTYEPPPTAPT |
| | | Wherein X is any amino acid that is not N |
| 335 | N18Q and R40A | ETRECIYYNANWELERTQQSGLERCEGEQDKRLHCYASWANS |
| | mutant of SEQ ID | SGTIELVKKGCWLDDFNCYDRQECVATEENPQVYFCCCEGNF |
| | NO: 1 | CNERFTHLPEAGGPEVTYEPPPTAPT |
| 336 | N18A mutant of | ETRECIYYNANWELERTAQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 337 | N18R mutant of | ETRECIYYNANWELERTRQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 338 | N18D mutant of | ETRECIYYNANWELERTDQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 339 | N18C mutant of | ETRECIYYNANWELERTCQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 340 | N18E mutant of | ETRECIYYNANWELERTEQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 341 | N18G mutant of | ETRECIYYNANWELERTGQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 342 | N18H mutant of | ETRECIYYNANWELERTHQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 343 | N18I mutant of | ETRECIYYNANWELERTIQSGLERCEGEQDKRLHCYASWRNSS |
| | SEQ ID NO: 1 | GTIELVKKGCWLDDFNCYDRQECVATEENPQVYFCCCEGNFC |
| | | NERFTHLPEAGGPEVTYEPPPTAPT |
| 344 | N18L mutant of | ETRECIYYNANWELERTLQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 345 | N18K mutant of | ETRECIYYNANWELERTKQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 346 | N18M mutant of | ETRECIYYNANWELERTMQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 347 | N18F mutant of | ETRECIYYNANWELERTFQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 348 | N18P mutant of | ETRECIYYNANWELERTPQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 349 | N18S mutant of | ETRECIYYNANWELERTSQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 350 | N18T mutant of | ETRECIYYNANWELERTTQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 351 | N18W mutant of | ETRECIYYNANWELERTWQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 352 | N18Y mutant of | ETRECIYYNANWELERTYQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 353 | N18V mutant of | ETRECIYYNANWELERTVQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 354 | N18A mutant of | ETRECIYYNANWELERTAQSGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 355 | S20A mutant of | ETRECIYYNANWELERTNQAGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 356 | S20R mutant of | ETRECIYYNANWELERTNQRGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 357 | S20N mutant of | ETRECIYYNANWELERTNQNGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 358 | S20D mutant of | ETRECIYYNANWELERTNQDGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 359 | S20C mutant of | ETRECIYYNANWELERTNQCGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 360 | S20Q mutant of | ETRECIYYNANWELERTNQQGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 361 | S20E mutant of | ETRECIYYNANWELERTNQEGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 362 | S20G mutant of | ETRECIYYNANWELERTNQGGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 363 | S20H mutant of | ETRECIYYNANWELERTNQHGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 364 | S20I mutant of | ETRECIYYNANWELERTNQIGLERCEGEQDKRLHCYASWRNSS |
| | SEQ ID NO: 1 | GTIELVKKGCWLDDFNCYDRQECVATEENPQVYFCCCEGNFC |
| | | NERFTHLPEAGGPEVTYEPPPTAPT |
| 365 | S20L mutant of | ETRECIYYNANWELERTNQLGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 366 | S20K mutant of | ETRECIYYNANWELERTNQKGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 367 | S20M mutant of | ETRECIYYNANWELERTNQMGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 368 | S20F mutant of | ETRECIYYNANWELERTNQFGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 369 | S20P mutant of | ETRECIYYNANWELERTNQPGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 370 | S20W mutant of | ETRECIYYNANWELERTNQWGLERCEGEQDKRLHCYASWRN |
| | SEQ ID NO: 1 | SSGTIELVKKGCWLDDFNCYDRQECVATEENPQVYFCCCEGN |
| | | FCNERFTHLPEAGGPEVTYEPPPTAPT |
| 371 | S20Y mutant of | ETRECIYYNANWELERTNQYGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 372 | S20V mutant of | ETRECIYYNANWELERTNQVGLERCEGEQDKRLHCYASWRNS |
| | SEQ ID NO: 1 | SGTIELVKKGCWLDDFNCYDRQECVATEENPQVYFCCCEGNF |
| | | CNERFTHLPEAGGPEVTYEPPPTAPT |
| 373 | S20X and L55D | ETRECIYYNANWELERTNQXGLERCEGEQDKRLHCYASWRNS |
| | mutant of SEQ ID | SGTIELVKKGCWDDDFNCYDRQECVATEENPQVYFCCCEGNF |
| | NO: 1 | CNERFTHLPEAGGPEVTYEPPPTAPT |
| | | Wherein X is any amino acid that is not S or T |
| 374 | S20X and L55E | ETRECIYYNANWELERTNQXGLERCEGEQDKRLHCYASWRNS |
| | mutant of SEQ ID | SGTIELVKKGCWEDDFNCYDRQECVATEENPQVYFCCCEGNF |
| | NO: 1 | CNERFTHLPEAGGPEVTYEPPPTAPT |
| | | Wherein X is any amino acid that is not S or T |
| 375 | S20X and R40A | ETRECIYYNANWELERTNQXGLERCEGEQDKRLHCYASWANS |
| | mutant of SEQ ID | SGTIELVKKGCWLDDFNCYDRQECVATEENPQVYFCCCEGNF |
| | NO: 1 | CNERFTHLPEAGGPEVTYEPPPTAPT |
| | | Wherein X is any amino acid that is not S or T |
| 376 | ActRIIB-ECD | ETRECIYYNANWELERTNQSGLERCEGDQDKRLHCYASWRNS |
| | fusion protein | SGTIELVKKGCWLDDINCYDRQECVATKENPQVYFCCCEGNF |
| | (without N18 or | CNERFTHLPEAGGPEVTYEPPPTAPTGGGGSESKYGPPCPPCPA |
| | S20 mutations) | PEFLGGPSVFLFPPKPKDTLMISRTPEVTCVVVDVSQEDPEVQF |
| | | NWYVDGVEVHNAKTKPREEQFNSTYRVVSVLTVLHQDWLNG |
| | | KEYKCKVSNKGLPSSIEKTISKAKGQPREPQVYTLPPSQEEMTK |
| | | NQVSLTCLVKGFYPSDIAVEWESNGQPENNYKTTPPVLDSDGS |
| | | FFLYSRLTVDKSRWQEGNVFSCSVMHEALHNHYTQKSLSLSL |
| | | G |
| 1651 | SEQ ID NO: 222 | MEFGLSWVFLVALLRGVQCETRECIYYNANWELERTQQSGLE |
| | with signal peptide | RCEGDQDKRLHCYASWRNSSGTIELVKKGCWLDDINCYDRQE |
| | (aa 1-19 is signal | CVATKENPQVYFCCCEGNFCNERFTHLPEAGGPEVTYEPPPTA |
| | peptide) | PTGGGGSESKYGPPCPPCPAPEFLGGPSVFLFPPKPKDTLMISRT |
| | | PEVTCVVVDVSQEDPEVQFNWYVDGVEVHNAKTKPREEQFNS |
| | | TYRVVSVLTVLHQDWLNGKEYKCKVSNKGLPSSIEKTISKAKG |
| | | QPREPQVYTLPPSQEEMTKNQVSLTCLVKGFYPSDIAVEWESN |
| | | GQPENNYKTTPPVLDSDGSFFLYSRLTVDKSRWQEGNVFSCSV |
| | | MHEALHNHYTQKSLSLSLG |
| 1652 | DNA sequence | ATGGAGTTTGGGCTGTCATGGGTTTTCCTCGTGGCCCTCCTC |
| | coding for SEQ ID | CGTGGAGTGCAGTGCGAAACTCGTGAGTGTATCTATTACAA |
| | NO: 1651 | CGCTAATTGGGAGCTCGAGCGGACTCAACAGAGCGGCCTTG |
| | | AACGATGTGAAGGGGATCAAGATAAACGTTTGCATTGTTAC |
| | | GCCAGTTGGAGGAACAGTTCCGGCACAATAGAGCTTGTCAA |
| | | GAAGGGTTGTTGGCTGGATGACATCAATTGTTATGATAGAC |
| | | AGGAATGTGTGGCTACAAAAGAAAACCCCCAAGTCTACTTT |
| | | TGCTGCTGTGAAGGGAATTTCTGTAATGAAAGATTTACCCA |
| | | CCTGCCAGAAGCAGGAGGACCCGAGGTAACATATGAGCCTC |
| | | CTCCAACAGCTCCCACAGGTGGTGGTGGTTCAGAATCCAAA |
| | | TATGGCCCACCATGTCCTCCTTGTCCCGCTCCTGAGTTCTTG |
| | | GGCGGACCCTCCGTCTTCCTGTTTCCCCCTAAGCCCAAGGAC |
| | | ACACTGATGATCTCTCGCACACCCGAAGTAACTTGTGTAGT |
| | | AGTTGACGTTTCTCAAGAGGATCCTGAAGTTCAGTTCAATTG |
| | | GTACGTCGATGGTGTGGAGGTGCACAACGCTAAAACAAAGC |
| | | CCCGCGAGGAGCAATTCAACTCCACTTATCGTGTCGTAAGC |
| | | GTACTCACCGTACTTCATCAAGACTGGCTGAATGGGAAAGA |
| | | ATACAAATGCAAGGTGTCTAATAAAGGCCTGCCATCTAGTA |
| | | TCGAGAAAACTATTTCCAAAGCCAAAGGCCAACCACGAGAG |
| | | CCCCAGGTCTATACTCTCCCTCCCTCTCAAGAAGAGATGACT |
| | | AAGAACCAGGTATCACTGACTTGTCTTGTCAAAGGATTTTAC |
| | | CCTAGCGATATCGCTGTAGAATGGGAAAGTAATGGGCAGCC |
| | | CGAGAACAACTATAAGACTACCCCTCCCGTTCTGGATAGCG |
| | | ACGGCTCTTTTTTTTTGTACAGTAGGCTTACAGTGGACAAGT |
| | | CCCGATGGCAAGAGGGCAATGTTTTTTCTTGTTCAGTGATGC |
| | | ACGAAGCACTGCATAACCACTATACACAAAAGTCTCTTTCC |
| | | TTGTCTTTGGGTTGA |
| 1653 | DNA sequence | GAAACTCGTGAGTGTATCTATTACAACGCTAATTGGGAGCT |
| | coding for SEQ ID | CGAGCGGACTCAACAGAGCGGCCTTGAACGATGTGAAGGG |
| | NO: 222 | GATCAAGATAAACGTTTGCATTGTTACGCCAGTTGGAGGAA |
| | | CAGTTCCGGCACAATAGAGCTTGTCAAGAAGGGTTGTTGGC |
| | | TGGATGACATCAATTGTTATGATAGACAGGAATGTGTGGCT |
| | | ACAAAAGAAAACCCCCAAGTCTACTTTTGCTGCTGTGAAGG |
| | | GAATTTCTGTAATGAAAGATTTACCCACCTGCCAGAAGCAG |
| | | GAGGACCCGAGGTAACATATGAGCCTCCTCCAACAGCTCCC |
| | | ACAGGTGGTGGTGGTTCAGAATCCAAATATGGCCCACCATG |
| | | TCCTCCTTGTCCCGCTCCTGAGTTCTTGGGCGGACCCTCCGT |
| | | CTTCCTGTTTCCCCCTAAGCCCAAGGACACACTGATGATCTC |
| | | TCGCACACCCGAAGTAACTTGTGTAGTAGTTGACGTTTCTCA |
| | | AGAGGATCCTGAAGTTCAGTTCAATTGGTACGTCGATGGTG |
| | | TGGAGGTGCACAACGCTAAAACAAAGCCCCGCGAGGAGCA |
| | | ATTCAACTCCACTTATCGTGTCGTAAGCGTACTCACCGTACT |
| | | TCATCAAGACTGGCTGAATGGGAAAGAATACAAATGCAAG |
| | | GTGTCTAATAAAGGCCTGCCATCTAGTATCGAGAAAACTAT |
| | | TTCCAAAGCCAAAGGCCAACCACGAGAGCCCCAGGTCTATA |
| | | CTCTCCCTCCCTCTCAAGAAGAGATGACTAAGAACCAGGTA |
| | | TCACTGACTTGTCTTGTCAAAGGATTTTACCCTAGCGATATC |
| | | GCTGTAGAATGGGAAAGTAATGGGCAGCCCGAGAACAACT |
| | | ATAAGACTACCCCTCCCGTTCTGGATAGCGACGGCTCTTTTT |
| | | TTTTGTACAGTAGGCTTACAGTGGACAAGTCCCGATGGCAA |
| | | GAGGGCAATGTTTTTTCTTGTTCAGTGATGCACGAAGCACTG |
| | | CATAACCACTATACACAAAAGTCTCTTTCCTTGTCTTTGGGT |
| | | TGA |
| 1654 | Truncated wild- | ETQECLFFNANWEKDRTNQTGVEPCYGDKDKRRHCFATWKNI |
| | type ActRIIA-ECD | SGSIEIVKQGCWLDDINCYDRTDCVEKKDSPEVYFCCCEGNMC |
| | (amino acids 7-110 | NEKFSYFPEMEVTQPTSNPVTPKPP |
| | of SEQ ID NO: 48) | |
| 1655 | N18X mutant of | ETQECLFFNANWEKDRTXQTGVEPCYGDKDKRRHCFATWKNI |
| | SEQ ID NO: 1654 | SGSIEIVKQGCWLDDINCYDRTDCVEKKDSPEVYFCCCEGNMC |
| | | NEKFSYFPEMEVTQPTSNPVTPKPP |
| | | wherein X is any amino acid other than N |
| 1656 | N18Q mutant of | ETQECLFFNANWEKDRTQQTGVEPCYGDKDKRRHCFATWKNI |
| | SEQ ID NO: 1654 | SGSIEIVKQGCWLDDINCYDRTDCVEKKDSPEVYFCCCEGNMC |
| | | NEKFSYFPEMEVTQPTSNPVTPKPP |
| 1657 | T20X mutant of | ETQECLFFNANWEKDRTNQXGVEPCYGDKDKRRHCFATWKN |
| | SEQ ID NO: 1654 | ISGSIEIVKQGCWLDDINCYDRTDCVEKKDSPEVYFCCCEGNM |
| | | CNEKFSYFPEMEVTQPTSNPVTPKPP |
| | | wherein X is any amino acid other than S or T |
| 1658 | ActRIIB-ECD | ETRECIYYNANWELERTNQSGLERCEGEQDKRLHCYASWRNS |
| | polypeptide in | SGTIELVKKGCWDDDFNCYDRQECVATEENPQVYFCCCEGNF |
| | Luspatercept | CNERFTHLPEAGGPEVTYEPPPT |
| 1659 | N18X mutation of | ETRECIYYNANWELERTXQSGLERCEGEQDKRLHCYASWRNS |
| | SEQ ID NO: 1658 | SGTIELVKKGCWDDDFNCYDRQECVATEENPQVYFCCCEGNF |
| | | CNERFTHLPEAGGPEVTYEPPPT |
| | | wherein X is any amino acid other than N |
| 1660 | N18Q mutation of | ETRECIYYNANWELERTQQSGLERCEGEQDKRLHCYASWRNS |
| | SEQ ID NO: 1658 | SGTIELVKKGCWDDDFNCYDRQECVATEENPQVYFCCCEGNF |
| | | CNERFTHLPEAGGPEVTYEPPPT |
| 1661 | S20X mutation of | TRECIYYNANWELERTNQXGLERCEGEQDKRLHCYASWRNSS |
| | SEQ ID NO: 1658 | GTIELVKKGCWDDDFNCYDRQECVATEENPQVYFCCCEGNFC |
| | | NERFTHLPEAGGPEVTYEPPPT |
| | | wherein X is any amino acid other than S or T |
| 1664 | N18X mutation of | ETRECIYYNANWELERTXQSGLERCEGEQDKRLHCYASWRNS |
| | luspatercept | SGTIELVKKGCWDDDFNCYDRQECVATEENPQVYFCCCEGNF |
| | | CNERFTHLPEAGGPEVTYEPPPTGGGTHTCPPCPAPELLGGPSV |
| | | FLFPPKPKDTLMISRTPEVTCVVVDVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSREEMTKNQ |
| | | VSLTCLVKGFYPSDIAVEWESNGQPENNYKTTPPVLDSDG |
| | | SFFLYSKLTVDKSRWQQGNVFSCSVMHEALHNHYTQKSLS |
| | | LSPGK |
| | | wherein X is any amino acid other than N |
| 1665 | N18X mutation of | ILGRSETQECLFFNANWEKDRTXQTGVEPCYGDKDKRRHCFA |
| | sotatercept | TWKNISGSIEIVKQGCWLDDINCYDRTDCVEKKDSPEVYFCCC |
| | | EGNMCNEKFSYFPEMEVTQPTSNPVTPKPPTGGGTHTCPPCPA |
| | | PELLGGPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPVPIEKTISKAKGQPREPQVYTLPPSREEMTK |
| | | NQVSLTCLVKGFYPSDIAVEWESNGQPENNYKTTPPVLDSDGS |
| | | FFLYSKLTVDKSRWQQGNVFSCSVMHEALHNHYTQKSLSLSP |
| | | GK |
| | | wherein X is any amino acid other than N |

TABLE 10
Additional Sequences (SEQ ID 377-1650)

| SEQ | | |
|---|---|---|
| ID | | |
| NO: | Notes | Sequences |
| 377 | N-terminal 5 aa truncation of | AETRECIYYNANWELERTNQSGLERCEGEQDKRLHCYASWRNSSGTIELVK |
| | Wild-type human ActRIIB | KGCWLDDFNCYDRQECVATEENPQVYFCCCEGNFCNERFTHLPEAGGPEVT |
| | extracellular domain (N- | YEPPPTAPT |
| | terminal 5 aa truncation of | |
| | SEQ ID NO: 46) | |
| 378 | N-terminal 4 aa truncation of | EAETRECIYYNANWELERTNQSGLERCEGEQDKRLHCYASWRNSSGTIELV |
| | Wild-type human ActRIIB | KKGCWLDDFNCYDRQECVATEENPQVYFCCCEGNFCNERFTHLPEAGGPEV |
| | extracellular domain (N- | TYEPPPTAPT |
| | terminal 4 aa truncation of | |
| | SEQ ID NO: 46) | |
| 379 | N-terminal 3 aa truncation of | GEAETRECIYYNANWELERTNQSGLERCEGEQDKRLHCYASWRNSSGTIEL |
| | Wild-type human ActRIIB | VKKGCWLDDFNCYDRQECVATEENPQVYFCCCEGNFCNERFTHLPEAGGPE |
| | extracellular domain (N- | VTYEPPPTAPT |
| | terminal 3 aa truncation of | |
| | SEQ ID NO: 46) | |
| 380 | N-terminal 2 aa truncation of | RGEAETRECIYYNANWELERTNQSGLERCEGEQDKRLHCYASWRNSSGTIE |
| | Wild-type human ActRIIB | LVKKGCWLDDFNCYDRQECVATEENPQVYFCCCEGNFCNERFTHLPEAGGP |
| | extracellular domain (N- | EVTYEPPPTAPT |
| | terminal 2 aa truncation of | |
| | SEQ ID NO: 46) | |
| 381 | N-terminal 1 aa truncation of | GRGEAETRECIYYNANWELERTNQSGLERCEGEQDKRLHCYASWRNSSGTI |
| | Wild-type human ActRIIB | ELVKKGCWLDDFNCYDRQECVATEENPQVYFCCCEGNFCNERFTHLPEAGG |
| | extracellular domain (N- | PEVTYEPPPTAPT |
| | terminal 1 aa truncation of | |
| | SEQ ID NO: 46) | |
| 382 | N19Q mutation of N- | AETRECIYYNANWELERTQQSGLERCEGEQDKRLHCYASWRNSSGTIELVK |
| | terminal 5 aa truncation of | KGCWLDDFNCYDRQECVATEENPQVYFCCCEGNFCNERFTHLPEAGGPEVT |
| | Wild-type human ActRIIB | YEPPPTAPT |
| | extracellular domain (N- | |
| | terminal 5 aa truncation of | |
| | SEQ ID NO: 46) | |
| 383 | N20Q mutation of N- | EAETRECIYYNANWELERTQQSGLERCEGEQDKRLHCYASWRNSSGTIELV |
| | terminal 4 aa truncation of | KKGCWLDDFNCYDRQECVATEENPQVYFCCCEGNFCNERFTHLPEAGGPEV |
| | Wild-type human ActRIIB | TYEPPPTAPT |
| | extracellular domain (N- | |
| | terminal 4 aa truncation of | |
| | SEQ ID NO: 46) | |
| 384 | N21Q mutation of N- | GEAETRECIYYNANWELERTQQSGLERCEGEQDKRLHCYASWRNSSGTIEL |
| | terminal 3 aa truncation of | VKKGCWLDDFNCYDRQECVATEENPQVYFCCCEGNFCNERFTHLPEAGGPE |
| | Wild-type human ActRIIB | VTYEPPPTAPT |
| | extracellular domain (N- | |
| | terminal 3 aa truncation of | |
| | SEQ ID NO: 46) | |
| 385 | N22Q mutation of N- | RGEAETRECIYYNANWELERTQQSGLERCEGEQDKRLHCYASWRNSSGTIE |
| | terminal 2 aa truncation of | LVKKGCWLDDFNCYDRQECVATEENPQVYFCCCEGNFCNERFTHLPEAGGP |
| | Wild-type human ActRIIB | EVTYEPPPTAPT |
| | extracellular domain (N- | |
| | terminal 2 aa truncation of | |
| | SEQ ID NO: 46) | |
| 386 | N23Q mutation of N- | GRGEAETRECIYYNANWELERTQQSGLERCEGEQDKRLHCYASWRNSSGTI |
| | terminal 1 aa truncation of | ELVKKGCWLDDFNCYDRQECVATEENPQVYFCCCEGNFCNERFTHLPEAGG |
| | Wild-type human ActRIIB | PEVTYEPPPTAPT |
| | extracellular domain (N- | |
| | terminal 1 aa truncation of | |
| | SEQ ID NO: 46) | |
| 387 | N24Q mutation of Wild-type | SGRGEAETRECIYYNANWELERTQQSGLERCEGEQDKRLHCYASWRNSSGT |
| | human ActRIIB extracellular | IELVKKGCWLDDFNCYDRQECVATEENPQVYFCCCEGNFCNERFTHLPEAG |
| | domain (N-terminal 1 aa | GPEVTYEPPPTAPT |
| | truncation of SEQ ID NO: | |
| | 46) | |
| 1666 | Hybrid hu-ActRIIB-ECD | AETRECIYYNANWELERTNQSGLERCYGDKDKRRHCYASWRNSSGTIELVK |
| | (SEQ ID NO: 3) with 1 | KGCWLDDINCYDRTDCVEKKDSPEVYFCCCEGNFCNERFTHLPEAGGPEVT |
| | additional aa at N-terminus | YEPPPTAPT |
| 388 | Hybrid hu-ActRIIB-ECD | EAETRECIYYNANWELERTNQSGLERCYGDKDKRRHCYASWRNSSGTIELV |
| | (SEQ ID NO: 3) with 2 | KKGCWLDDINCYDRTDCVEKKDSPEVYFCCCEGNFCNERFTHLPEAGGPEV |
| | additional aa at N-terminus | TYEPPPTAPT |
| 389 | Hybrid hu-ActRIIB-ECD | GEAETRECIYYNANWELERTNQSGLERCYGDKDKRRHCYASWRNSSGTIEL |
| | (SEQ ID NO: 3) with 3 | VKKGCWLDDINCYDRTDCVEKKDSPEVYFCCCEGNFCNERFTHLPEAGGPE |
| | additional aa at N-terminus | VTYEPPPTAPT |
| 390 | Hybrid hu-ActRIIB-ECD | RGEAETRECIYYNANWELERTNQSGLERCYGDKDKRRHCYASWRNSSGTIE |
| | (SEQ ID NO: 3) with 4 | LVKKGCWLDDINCYDRTDCVEKKDSPEVYFCCCEGNFCNERFTHLPEAGGP |
| | additional aa at N-terminus | EVTYEPPPTAPT |
| 391 | Hybrid hu-ActRIIB-ECD | GRGEAETRECIYYNANWELERTNQSGLERCYGDKDKRRHCYASWRNSSGTI |
| | (SEQ ID NO: 3) with 5 | ELVKKGCWLDDINCYDRTDCVEKKDSPEVYFCCCEGNFCNERFTHLPEAGG |
| | additional aa at N-terminus | PEVTYEPPPTAPT |
| 392 | Hybrid hu-ActRIIB-ECD | SGRGEAETRECIYYNANWELERTNQSGLERCYGDKDKRRHCYASWRNSSG |
| | (SEQ ID NO: 3) with 6 | TIELVKKGCWLDDINCYDRTDCVEKKDSPEVYFCCCEGNFCNERFTHLPEAG |
| | additional aa at N-terminus | GPEVTYEPPPTAPT |
| 393 | Hybrid hu-ActRIIB-ECD | AETRECIYYNANWELERTNQSGLERCYGDKDKRRHCYASWRNSSGTIELVK |
| | (SEQ ID NO: 4) with 1 | KGCWLDDFNCYDRTDCVEKKDSPEVYFCCCEGNFCNERFTHLPEAGGPEVT |
| | additional aa at N-terminus | YEPPPTAPT |
| 394 | Hybrid hu-ActRIIB-ECD | EAETRECIYYNANWELERTNQSGLERCYGDKDKRRHCYASWRNSSGTIELV |
| | (SEQ ID NO: 4) with 2 | KKGCWLDDFNCYDRTDCVEKKDSPEVYFCCCEGNFCNERFTHLPEAGGPEV |
| | additional aa at N-terminus | TYEPPPTAPT |
| 395 | Hybrid hu-ActRIIB-ECD | GEAETRECIYYNANWELERTNQSGLERCYGDKDKRRHCYASWRNSSGTIEL |
| | (SEQ ID NO: 4) with 3 | VKKGCWLDDFNCYDRTDCVEKKDSPEVYFCCCEGNFCNERFTHLPEAGGPE |
| | additional aa at N-terminus | VTYEPPPTAPT |
| 396 | Hybrid hu-ActRIIB-ECD | RGEAETRECIYYNANWELERTNQSGLERCYGDKDKRRHCYASWRNSSGTIE |
| | (SEQ ID NO: 4) with 4 | LVKKGCWLDDFNCYDRTDCVEKKDSPEVYFCCCEGNFCNERFTHLPEAGGP |
| | additional aa at N-terminus | EVTYEPPPTAPT |
| 397 | Hybrid hu-ActRIIB-ECD | GRGEAETRECIYYNANWELERTNQSGLERCYGDKDKRRHCYASWRNSSGTI |
| | (SEQ ID NO: 4) with 5 | ELVKKGCWLDDFNCYDRTDCVEKKDSPEVYFCCCEGNFCNERFTHLPEAGG |
| | additional aa at N-terminus | PEVTYEPPPTAPT |
| 398 | Hybrid hu-ActRIIB-ECD | SGRGEAETRECIYYNANWELERTNQSGLERCYGDKDKRRHCYASWRNSSG |
| | (SEQ ID NO: 4) with 6 | TIELVKKGCWLDDFNCYDRTDCVEKKDSPEVYFCCCEGNFCNERFTHLPEA |
| | additional aa at N-terminus | GGPEVTYEPPPTAPT |
| 399 | Hybrid hu-ActRIIB-ECD | AETRECIYYNANWELERTNQSGLERCEGEQDKRLHCYASWRNSSGTIELVK |
| | (SEQ ID NO: 5) with 1 | KGCWLDDINCYDRTDCVEKKDSPEVYFCCCEGNFCNERFTHLPEAGGPEVT |
| | additional aa at N-terminus | YEPPPTAPT |
| 400 | Hybrid hu-ActRIIB-ECD | EAETRECIYYNANWELERTNQSGLERCEGEQDKRLHCYASWRNSSGTIELV |
| | (SEQ ID NO: 5) with 2 | KKGCWLDDINCYDRTDCVEKKDSPEVYFCCCEGNFCNERFTHLPEAGGPEV |
| | additional aa at N-terminus | TYEPPPTAPT |
| 401 | Hybrid hu-ActRIIB-ECD | GEAETRECIYYNANWELERTNQSGLERCEGEQDKRLHCYASWRNSSGTIEL |
| | (SEQ ID NO: 5) with 3 | VKKGCWLDDINCYDRTDCVEKKDSPEVYFCCCEGNFCNERFTHLPEAGGPE |
| | additional aa at N-terminus | VTYEPPPTAPT |
| 402 | Hybrid hu-ActRIIB-ECD | RGEAETRECIYYNANWELERTNQSGLERCEGEQDKRLHCYASWRNSSGTIE |
| | (SEQ ID NO: 5) with 4 | LVKKGCWLDDINCYDRTDCVEKKDSPEVYFCCCEGNFCNERFTHLPEAGGP |
| | additional aa at N-terminus | EVTYEPPPTAPT |
| 403 | Hybrid hu-ActRIIB-ECD | GRGEAETRECIYYNANWELERTNQSGLERCEGEQDKRLHCYASWRNSSGTI |
| | (SEQ ID NO: 5) with 5 | ELVKKGCWLDDINCYDRTDCVEKKDSPEVYFCCCEGNFCNERFTHLPEAGG |
| | additional aa at N-terminus | PEVTYEPPPTAPT |
| 404 | Hybrid hu-ActRIIB-ECD | SGRGEAETRECIYYNANWELERTNQSGLERCEGEQDKRLHCYASWRNSSGT |
| | (SEQ ID NO: 5) with 6 | IELVKKGCWLDDINCYDRTDCVEKKDSPEVYFCCCEGNFCNERFTHLPEAG |
| | additional aa at N-terminus | GPEVTYEPPPTAPT |
| 405 | Hybrid hu-ActRIIB-ECD | AETRECIYYNANWELERTNQSGLERCEGEQDKRLHCYASWRNSSGTIELVK |
| | (SEQ ID NO: 6) with 1 | KGCWLDDINCYDRTDCVEKKDSPQVYFCCCEGNFCNERFTHLPEAGGPEVT |
| | additional aa at N-terminus | YEPPPTAPT |
| 406 | Hybrid hu-ActRIIB-ECD | EAETRECIYYNANWELERTNQSGLERCEGEQDKRLHCYASWRNSSGTIELV |
| | (SEQ ID NO: 6) with 2 | KKGCWLDDINCYDRTDCVEKKDSPQVYFCCCEGNFCNERFTHLPEAGGPEV |
| | additional aa at N-terminus | TYEPPPTAPT |
| 407 | Hybrid hu-ActRIIB-ECD | GEAETRECIYYNANWELERTNQSGLERCEGEQDKRLHCYASWRNSSGTIEL |
| | (SEQ ID NO: 6) with 3 | VKKGCWLDDINCYDRTDCVEKKDSPQVYFCCCEGNFCNERFTHLPEAGGPE |
| | additional aa at N-terminus | VTYEPPPTAPT |
| 408 | Hybrid hu-ActRIIB-ECD | RGEAETRECIYYNANWELERTNQSGLERCEGEQDKRLHCYASWRNSSGTIE |
| | (SEQ ID NO: 6) with 4 | LVKKGCWLDDINCYDRTDCVEKKDSPQVYFCCCEGNFCNERFTHLPEAGGP |
| | additional aa at N-terminus | EVTYEPPPTAPT |
| 409 | Hybrid hu-ActRIIB-ECD | GRGEAETRECIYYNANWELERTNQSGLERCEGEQDKRLHCYASWRNSSGTI |
| | (SEQ ID NO: 6) with 5 | ELVKKGCWLDDINCYDRTDCVEKKDSPQVYFCCCEGNFCNERFTHLPEAGG |
| | additional aa at N-terminus | PEVTYEPPPTAPT |
| 410 | Hybrid hu-ActRIIB-ECD | SGRGEAETRECIYYNANWELERTNQSGLERCEGEQDKRLHCYASWRNSSGT |
| | (SEQ ID NO: 6) with 6 | IELVKKGCWLDDINCYDRTDCVEKKDSPQVYFCCCEGNFCNERFTHLPEAG |
| | additional aa at N-terminus | GPEVTYEPPPTAPT |
| 411 | Hybrid hu-ActRIIB-ECD | AETRECIYYNANWELERTNQSGLERCEGEQDKRLHCYASWRNSSGTIELVK |
| | (SEQ ID NO: 7) with 1 | KGCWLDDFNCYDRTDCVEKKDSPQVYFCCCEGNFCNERFTHLPEAGGPEVT |
| | additional aa at N-terminus | YEPPPTAPT |
| 412 | Hybrid hu-ActRIIB-ECD | EAETRECIYYNANWELERTNQSGLERCEGEQDKRLHCYASWRNSSGTIELV |
| | (SEQ ID NO: 7) with 2 | KKGCWLDDFNCYDRTDCVEKKDSPQVYFCCCEGNFCNERFTHLPEAGGPEV |
| | additional aa at N-terminus | TYEPPPTAPT |
| 413 | Hybrid hu-ActRIIB-ECD | GEAETRECIYYNANWELERTNQSGLERCEGEQDKRLHCYASWRNSSGTIEL |
| | (SEQ ID NO: 7) with 3 | VKKGCWLDDFNCYDRTDCVEKKDSPQVYFCCCEGNFCNERFTHLPEAGGPE |
| | additional aa at N-terminus | VTYEPPPTAPT |
| 414 | Hybrid hu-ActRIIB-ECD | RGEAETRECIYYNANWELERTNQSGLERCEGEQDKRLHCYASWRNSSGTIE |
| | (SEQ ID NO: 7) with 4 | LVKKGCWLDDFNCYDRTDCVEKKDSPQVYFCCCEGNFCNERFTHLPEAGGP |
| | additional aa at N-terminus | EVTYEPPPTAPT |
| 415 | Hybrid hu-ActRIIB-ECD | GRGEAETRECIYYNANWELERTNQSGLERCEGEQDKRLHCYASWRNSSGTI |
| | (SEQ ID NO: 7) with 5 | ELVKKGCWLDDFNCYDRTDCVEKKDSPQVYFCCCEGNFCNERFTHLPEAG |
| | additional aa at N-terminus | GPEVTYEPPPTAPT |
| 416 | Hybrid hu-ActRIIB-ECD | SGRGEA |
| | (SEQ ID NO: 7) with 6 | ETRECIYYNANWELERTNQSGLERCEGEQDKRLHCYASWRNSSGTIELVKK |
| | additional aa at N-terminus | GCWLDDFNCYDRTDCVEKKDSPQVYFCCCEGNFCNERFTHLPEAGGPEVTY |
| | | EPPPTAPT |
| 417 | Hybrid hu-ActRIIB-ECD | AETRECIYYNANWELERTNQSGLERCEGEQDKRLHCYASWRNSSGTIELVK |
| | (SEQ ID NO: 8) with 1 | KGCWLDDFNCYDRTDCVEKKDSPEVYFCCCEGNFCNERFTHLPEAGGPEVT |
| | additional aa at N-terminus | YEPPPTAPT |
| 418 | Hybrid hu-ActRIIB-ECD | EAETRECIYYNANWELERTNQSGLERCEGEQDKRLHCYASWRNSSGTIELV |
| | (SEQ ID NO: 8) with 2 | KKGCWLDDFNCYDRTDCVEKKDSPEVYFCCCEGNFCNERFTHLPEAGGPEV |
| | additional aa at N-terminus | TYEPPPTAPT |
| 419 | Hybrid hu-ActRIIB-ECD | GEAETRECIYYNANWELERTNQSGLERCEGEQDKRLHCYASWRNSSGTIEL |
| | (SEQ ID NO: 8) with 3 | VKKGCWLDDFNCYDRTDCVEKKDSPEVYFCCCEGNFCNERFTHLPEAGGPE |
| | additional aa at N-terminus | VTYEPPPTAPT |
| 420 | Hybrid hu-ActRIIB-ECD | RGEAETRECIYYNANWELERTNQSGLERCEGEQDKRLHCYASWRNSSGTIE |
| | (SEQ ID NO: 8) with 4 | LVKKGCWLDDFNCYDRTDCVEKKDSPEVYFCCCEGNFCNERFTHLPEAGGP |
| | additional aa at N-terminus | EVTYEPPPTAPT |
| 421 | Hybrid hu-ActRIIB-ECD | GRGEAETRECIYYNANWELERTNQSGLERCEGEQDKRLHCYASWRNSSGTI |
| | (SEQ ID NO: 8) with 5 | ELVKKGCWLDDFNCYDRTDCVEKKDSPEVYFCCCEGNFCNERFTHLPEAGG |
| | additional aa at N-terminus | PEVTYEPPPTAPT |
| 422 | Hybrid hu-ActRIIB-ECD | SGRGEAETRECIYYNANWELERTNQSGLERCEGEQDKRLHCYASWRNSSGT |
| | (SEQ ID NO: 8) with 6 | IELVKKGCWLDDFNCYDRTDCVEKKDSPEVYFCCCEGNFCNERFTHLPEAG |
| | additional aa at N-terminus | GPEVTYEPPPTAPT |
| 423 | Hybrid hu-ActRIIB-ECD | AETRECIYYNANWELERTNQSGLERCEGEQDKRLHCYASWRNSSGTIELVK |
| | (SEQ ID NO: 9) with 1 | KGCWLDDFNCYDRQECVEKKDSPEVYFCCCEGNFCNERFTHLPEAGGPEVT |
| | additional aa at N-terminus | YEPPPTAPT |
| 424 | Hybrid hu-ActRIIB-ECD | EAETRECIYYNANWELERTNQSGLERCEGEQDKRLHCYASWRNSSGTIELV |
| | (SEQ ID NO: 9) with 2 | KKGCWLDDFNCYDRQECVEKKDSPEVYFCCCEGNFCNERFTHLPEAGGPEV |
| | additional aa at N-terminus | TYEPPPTAPT |
| 425 | Hybrid hu-ActRIIB-ECD | GEAETRECIYYNANWELERTNQSGLERCEGEQDKRLHCYASWRNSSGTIEL |
| | (SEQ ID NO: 9) with 3 | VKKGCWLDDFNCYDRQECVEKKDSPEVYFCCCEGNFCNERFTHLPEAGGPE |
| | additional aa at N-terminus | VTYEPPPTAPT |
| 426 | Hybrid hu-ActRIIB-ECD | RGEAETRECIYYNANWELERTNQSGLERCEGEQDKRLHCYASWRNSSGTIE |
| | (SEQ ID NO: 9) with 4 | LVKKGCWLDDFNCYDRQECVEKKDSPEVYFCCCEGNFCNERFTHLPEAGGP |
| | additional aa at N-terminus | EVTYEPPPTAPT |
| 427 | Hybrid hu-ActRIIB-ECD | GRGEAETRECIYYNANWELERTNQSGLERCEGEQDKRLHCYASWRNSSGTI |
| | (SEQ ID NO: 9) with 5 | ELVKKGCWLDDFNCYDRQECVEKKDSPEVYFCCCEGNFCNERFTHLPEAGG |
| | additional aa at N-terminus | PEVTYEPPPTAPT |
| 428 | Hybrid hu-ActRIIB-ECD | SGRGEAETRECIYYNANWELERTNQSGLERCEGEQDKRLHCYASWRNSSGT |
| | (SEQ ID NO: 9) with 6 | IELVKKGCWLDDFNCYDRQECVEKKDSPEVYFCCCEGNFCNERFTHLPEAG |
| | additional aa at N-terminus | GPEVTYEPPPTAPT |
| 429 | Hybrid hu-ActRIIB-ECD | AETRECIYYNANWELERTNQSGLERCEGEQDKRLHCYASWRNSSGTIELVK |
| | (SEQ ID NO: 10) with 1 | KGCWLDDFNCYDRQECVEKKDSPQVYFCCCEGNFCNERFTHLPEAGGPEVT |
| | additional aa at N-terminus | YEPPPTAPT |
| 430 | Hybrid hu-ActRIIB-ECD | EAETRECIYYNANWELERTNQSGLERCEGEQDKRLHCYASWRNSSGTIELV |
| | (SEQ ID NO: 10) with 2 | KKGCWLDDFNCYDRQECVEKKDSPQVYFCCCEGNFCNERFTHLPEAGGPEV |
| | additional aa at N-terminus | TYEPPPTAPT |
| 431 | Hybrid hu-ActRIIB-ECD | GEAETRECIYYNANWELERTNQSGLERCEGEQDKRLHCYASWRNSSGTIEL |
| | (SEQ ID NO: 10) with 3 | VKKGCWLDDFNCYDRQECVEKKDSPQVYFCCCEGNFCNERFTHLPEAGGPE |
| | additional aa at N-terminus | VTYEPPPTAPT |
| 432 | Hybrid hu-ActRIIB-ECD | RGEAETRECIYYNANWELERTNQSGLERCEGEQDKRLHCYASWRNSSGTIE |
| | (SEQ ID NO: 10) with 4 | LVKKGCWLDDFNCYDRQECVEKKDSPQVYFCCCEGNFCNERFTHLPEAGGP |
| | additional aa at N-terminus | EVTYEPPPTAPT |
| 433 | Hybrid hu-ActRIIB-ECD | GRGEAETRECIYYNANWELERTNQSGLERCEGEQDKRLHCYASWRNSSGTI |
| | (SEQ ID NO: 10) with 5 | ELVKKGCWLDDFNCYDRQECVEKKDSPQVYFCCCEGNFCNERFTHLPEAG |
| | additional aa at N-terminus | GPEVTYEPPPTAPT |
| 434 | Hybrid hu-ActRIIB-ECD | SGRGEAETRECIYYNANWELERTNQSGLERCEGEQDKRLHCYASWRNSSGT |
| | (SEQ ID NO: 10) with 6 | IELVKKGCWLDDFNCYDRQECVEKKDSPQVYFCCCEGNFCNERFTHLPEAG |
| | additional aa at N-terminus | GPEVTYEPPPTAPT |
| 435 | Hybrid hu-ActRIIB-ECD | ATRECIYYNANWELERTNQSGLERCEGEQDKRLHCYASWRNSSGTIELVKK |
| | (SEQ ID NO: 11) with 1 | GCWLDDINCYDRQECVEKKDSPEVYFCCCEGNFCNERFTHLPEAGGPEVTY |
| | additional aa at N-terminus | EPPPTAPT |
| 436 | Hybrid hu-ActRIIB-ECD | EATRECIYYNANWELERTNQSGLERCEGEQDKRLHCYASWRNSSGTIELVK |
| | (SEQ ID NO: 11) with 2 | KGCWLDDINCYDRQECVEKKDSPEVYFCCCEGNFCNERFTHLPEAGGPEVT |
| | additional aa at N-terminus | YEPPPTAPT |
| 437 | Hybrid hu-ActRIIB-ECD | GEATRECIYYNANWELERTNQSGLERCEGEQDKRLHCYASWRNSSGTIELV |
| | (SEQ ID NO: 11) with 3 | KKGCWLDDINCYDRQECVEKKDSPEVYFCCCEGNFCNERFTHLPEAGGPEV |
| | additional aa at N-terminus | TYEPPPTAPT |
| 438 | Hybrid hu-ActRIIB-ECD | RGEATRECIYYNANWELERTNQSGLERCEGEQDKRLHCYASWRNSSGTIEL |
| | (SEQ ID NO: 11) with 4 | VKKGCWLDDINCYDRQECVEKKDSPEVYFCCCEGNFCNERFTHLPEAGGPE |
| | additional aa at N-terminus | VTYEPPPTAPT |
| 439 | Hybrid hu-ActRIIB-ECD | GRGEATRECIYYNANWELERTNQSGLERCEGEQDKRLHCYASWRNSSGTIE |
| | (SEQ ID NO: 11) with 5 | LVKKGCWLDDINCYDRQECVEKKDSPEVYFCCCEGNFCNERFTHLPEAGGP |
| | additional aa at N-terminus | EVTYEPPPTAPT |
| 440 | Hybrid hu-ActRIIB-ECD | SGRGEATRECIYYNANWELERTNQSGLERCEGEQDKRLHCYASWRNSSGTI |
| | (SEQ ID NO: 11) with 6 | ELVKKGCWLDDINCYDRQECVEKKDSPEVYFCCCEGNFCNERFTHLPEAGG |
| | additional aa at N-terminus | PEVTYEPPPTAPT |
| 441 | Hybrid hu-ActRIIB-ECD | AETRECIYYNANWELERTNQSGLERCEGEQDKRLHCYASWRNSSGTIELVK |
| | (SEQ ID NO: 12) with 1 | KGCWLDDFNCYDRTDCVEKKDSPEVYFCCCEGNMCNERFTHLPEAGGPEV |
| | additional aa at N-terminus | TYEPPPTAPT |
| 442 | Hybrid hu-ActRIIB-ECD | EAETRECIYYNANWELERTNQSGLERCEGEQDKRLHCYASWRNSSGTIELV |
| | (SEQ ID NO: 12) with 2 | KKGCWLDDFNCYDRTDCVEKKDSPEVYFCCCEGNMCNERFTHLPEAGGPE |
| | additional aa at N-terminus | VTYEPPPTAPT |
| 443 | Hybrid hu-ActRIIB-ECD | GEAETRECIYYNANWELERTNQSGLERCEGEQDKRLHCYASWRNSSGTIEL |
| | (SEQ ID NO: 12) with 3 | VKKGCWLDDFNCYDRTDCVEKKDSPEVYFCCCEGNMCNERFTHLPEAGGP |
| | additional aa at N-terminus | EVTYEPPPTAPT |
| 444 | Hybrid hu-ActRIIB-ECD | RGEAETRECIYYNANWELERTNQSGLERCEGEQDKRLHCYASWRNSSGTIE |
| | (SEQ ID NO: 12) with 4 | LVKKGCWLDDFNCYDRTDCVEKKDSPEVYFCCCEGNMCNERFTHLPEAGG |
| | additional aa at N-terminus | PEVTYEPPPTAPT |
| 445 | Hybrid hu-ActRIIB-ECD | GRGEAETRECIYYNANWELERTNQSGLERCEGEQDKRLHCYASWRNSSGTI |
| | (SEQ ID NO: 12) with 5 | ELVKKGCWLDDFNCYDRTDCVEKKDSPEVYFCCCEGNMCNERFTHLPEAG |
| | additional aa at N-terminus | GPEVTYEPPPTAPT |
| 446 | Hybrid hu-ActRIIB-ECD | SGRGEAETRECIYYNANWELERTNQSGLERCEGEQDKRLHCYASWRNSSGT |
| | (SEQ ID NO: 12) with 6 | IELVKKGCWLDDFNCYDRTDCVEKKDSPEVYFCCCEGNMCNERFTHLPEAG |
| | additional aa at N-terminus | GPEVTYEPPPTAPT |
| 447 | Hybrid hu-ActRIIB-ECD | AETRECIYYNANWELERTNQSGLERCEGDQDKRLHCYASWRNSSGTIELVK |
| | (SEQ ID NO: 13) with 1 | KGCWLDDFNCYDRQECVEKKDSPEVYFCCCEGNMCNERFTHLPEAGGPEV |
| | additional aa at N-terminus | TYEPPPTAPT |
| 448 | Hybrid hu-ActRIIB-ECD | EAETRECIYYNANWELERTNQSGLERCEGDQDKRLHCYASWRNSSGTIELV |
| | (SEQ ID NO: 13) with 2 | KKGCWLDDFNCYDRQECVEKKDSPEVYFCCCEGNMCNERFTHLPEAGGPE |
| | additional aa at N-terminus | VTYEPPPTAPT |
| 449 | Hybrid hu-ActRIIB-ECD | GEAETRECIYYNANWELERTNQSGLERCEGDQDKRLHCYASWRNSSGTIEL |
| | (SEQ ID NO: 13) with 3 | VKKGCWLDDFNCYDRQECVEKKDSPEVYFCCCEGNMCNERFTHLPEAGGP |
| | additional aa at N-terminus | EVTYEPPPTAPT |
| 450 | Hybrid hu-ActRIIB-ECD | RGEAETRECIYYNANWELERTNQSGLERCEGDQDKRLHCYASWRNSSGTIE |
| | (SEQ ID NO: 13) with 4 | LVKKGCWLDDFNCYDRQECVEKKDSPEVYFCCCEGNMCNERFTHLPEAGG |
| | additional aa at N-terminus | PEVTYEPPPTAPT |
| 451 | Hybrid hu-ActRIIB-ECD | GRGEAETRECIYYNANWELERTNQSGLERCEGDQDKRLHCYASWRNSSGTI |
| | (SEQ ID NO: 13) with 5 | ELVKKGCWLDDFNCYDRQECVEKKDSPEVYFCCCEGNMCNERFTHLPEAG |
| | additional aa at N-terminus | GPEVTYEPPPTAPT |
| 452 | Hybrid hu-ActRIIB-ECD | SGRGEAETRECIYYNANWELERTNQSGLERCEGDQDKRLHCYASWRNSSGT |
| | (SEQ ID NO: 13) with 6 | IELVKKGCWLDDFNCYDRQECVEKKDSPEVYFCCCEGNMCNERFTHLPEAG |
| | additional aa at N-terminus | GPEVTYEPPPTAPT |
| 453 | Hybrid hu-ActRIIB-ECD | AETQECIYYNANWEKDRTNQTGVEPCYGDKDKRRHCYASWRNSSGTIELV |
| | (SEQ ID NO: 14) with 1 | KKGCWLDDFNCYDRQECVATEENPQVYFCCCEGNFCNERFTHLPEAGGPEV |
| | additional aa at N-terminus | TYEPPPTAPT |
| 454 | Hybrid hu-ActRIIB-ECD | EAETQECIYYNANWEKDRTNQTGVEPCYGDKDKRRHCYASWRNSSGTIELV |
| | (SEQ ID NO: 14) with 2 | KKGCWLDDFNCYDRQECVATEENPQVYFCCCEGNFCNERFTHLPEAGGPEV |
| | additional aa at N-terminus | TYEPPPTAPT |
| 455 | Hybrid hu-ActRIIB-ECD | GEAETQECIYYNANWEKDRTNQTGVEPCYGDKDKRRHCYASWRNSSGTIEL |
| | (SEQ ID NO: 14) with 3 | VKKGCWLDDFNCYDRQECVATEENPQVYFCCCEGNFCNERFTHLPEAGGPE |
| | additional aa at N-terminus | VTYEPPPTAPT |
| 456 | Hybrid hu-ActRIIB-ECD | RGEAETQECIYYNANWEKDRTNQTGVEPCYGDKDKRRHCYASWRNSSGTI |
| | (SEQ ID NO: 14) with 4 | ELVKKGCWLDDFNCYDRQECVATEENPQVYFCCCEGNFCNERFTHLPEAGG |
| | additional aa at N-terminus | PEVTYEPPPTAPT |
| 457 | Hybrid hu-ActRIIB-ECD | GRGEAETQECIYYNANWEKDRTNQTGVEPCYGDKDKRRHCYASWRNSSGT |
| | (SEQ ID NO: 14) with 5 | IELVKKGCWLDDFNCYDRQECVATEENPQVYFCCCEGNFCNERFTHLPEAG |
| | additional aa at N-terminus | GPEVTYEPPPTAPT |
| 458 | Hybrid hu-ActRIIB-ECD | SGRGEAETQECIYYNANWEKDRTNQTGVEPCYGDKDKRRHCYASWRNSSG |
| | (SEQ ID NO: 14) with 6 | TIELVKKGCWLDDFNCYDRQECVATEENPQVYFCCCEGNFCNERFTHLPEA |
| | additional aa at N-terminus | GGPEVTYEPPPTAPT |
| 459 | Hybrid hu-ActRIIB-ECD | AETQECIYYNANWEKDRTNQTGVEPCEGDQDKRLHCYASWRNSSGTIELVK |
| | (SEQ ID NO: 15) with 1 | KGCWLDDFNCYDRQECVATEENPQVYFCCCEGNFCNERFTHLPEAGGPEVT |
| | additional aa at N-terminus | YEPPPTAPT |
| 460 | Hybrid hu-ActRIIB-ECD | EAETQECIYYNANWEKDRTNQTGVEPCEGDQDKRLHCYASWRNSSGTIELV |
| | (SEQ ID NO: 15) with 2 | KKGCWLDDFNCYDRQECVATEENPQVYFCCCEGNFCNERFTHLPEAGGPEV |
| | additional aa at N-terminus | TYEPPPTAPT |
| 461 | Hybrid hu-ActRIIB-ECD | GEAETQECIYYNANWEKDRTNQTGVEPCEGDQDKRLHCYASWRNSSGTIEL |
| | (SEQ ID NO: 15) with 3 | VKKGCWLDDFNCYDRQECVATEENPQVYFCCCEGNFCNERFTHLPEAGGPE |
| | additional aa at N-terminus | VTYEPPPTAPT |
| 462 | Hybrid hu-ActRIIB-ECD | RGEAETQECIYYNANWEKDRTNQTGVEPCEGDQDKRLHCYASWRNSSGTIE |
| | (SEQ ID NO: 15) with 4 | LVKKGCWLDDFNCYDRQECVATEENPQVYFCCCEGNFCNERFTHLPEAGGP |
| | additional aa at N-terminus | EVTYEPPPTAPT |
| 463 | Hybrid hu-ActRIIB-ECD | GRGEAETQECIYYNANWEKDRTNQTGVEPCEGDQDKRLHCYASWRNSSGTI |
| | (SEQ ID NO: 15) with 5 | ELVKKGCWLDDFNCYDRQECVATEENPQVYFCCCEGNFCNERFTHLPEAGG |
| | additional aa at N-terminus | PEVTYEPPPTAPT |
| 464 | Hybrid hu-ActRIIB-ECD | SGRGEAETQECIYYNANWEKDRTNQTGVEPCEGDQDKRLHCYASWRNSSG |
| | (SEQ ID NO: 15) with 6 | TIELVKKGCWLDDFNCYDRQECVATEENPQVYFCCCEGNFCNERFTHLPEA |
| | additional aa at N-terminus | GGPEVTYEPPPTAPT |
| 465 | Hybrid hu-ActRIIB-ECD | AETRECIYYNANWELERTNQSGLERCYGDKDKRRHCYASWRNSSGTIELVK |
| | (SEQ ID NO: 16) with 1 | KGCWLDDFNCYDRQECVATEENPQVYFCCCEGNFCNERFTHLPEAGGPEVT |
| | additional aa at N-terminus | YEPPPTAPT |
| 466 | Hybrid hu-ActRIIB-ECD | EAETRECIYYNANWELERTNQSGLERCYGDKDKRRHCYASWRNSSGTIELV |
| | (SEQ ID NO: 16) with 2 | KKGCWLDDFNCYDRQECVATEENPQVYFCCCEGNFCNERFTHLPEAGGPEV |
| | additional aa at N-terminus | TYEPPPTAPT |
| 467 | Hybrid hu-ActRIIB-ECD | GEAETRECIYYNANWELERTNQSGLERCYGDKDKRRHCYASWRNSSGTIEL |
| | (SEQ ID NO: 16) with 3 | VKKGCWLDDFNCYDRQECVATEENPQVYFCCCEGNFCNERFTHLPEAGGPE |
| | additional aa at N-terminus | VTYEPPPTAPT |
| 468 | Hybrid hu-ActRIIB-ECD | RGEAETRECIYYNANWELERTNQSGLERCYGDKDKRRHCYASWRNSSGTIE |
| | (SEQ ID NO: 16) with 4 | LVKKGCWLDDFNCYDRQECVATEENPQVYFCCCEGNFCNERFTHLPEAGGP |
| | additional aa at N-terminus | EVTYEPPPTAPT |
| 469 | Hybrid hu-ActRIIB-ECD | GRGEAETRECIYYNANWELERTNQSGLERCYGDKDKRRHCYASWRNSSGTI |
| | (SEQ ID NO: 16) with 5 | ELVKKGCWLDDFNCYDRQECVATEENPQVYFCCCEGNFCNERFTHLPEAGG |
| | additional aa at N-terminus | PEVTYEPPPTAPT |
| 470 | Hybrid hu-ActRIIB-ECD | SGRGEAETRECIYYNANWELERTNQSGLERCYGDKDKRRHCYASWRNSSG |
| | (SEQ ID NO: 16) with 6 | TIELVKKGCWLDDFNCYDRQECVATEENPQVYFCCCEGNFCNERFTHLPEA |
| | additional aa at N-terminus | GGPEVTYEPPPTAPT |
| 471 | Hybrid hu-ActRIIB-ECD | AETRECIYYNANWEKDRTNQTGVEPCEGDQDKRLHCYASWRNSSGTIELVK |
| | (SEQ ID NO: 17) with 1 | KGCWLDDFNCYDRQECVATEENPQVYFCCCEGNFCNERFTHLPEAGGPEVT |
| | additional aa at N-terminus | YEPPPTAPT |
| 472 | Hybrid hu-ActRIIB-ECD | EAETRECIYYNANWEKDRTNQTGVEPCEGDQDKRLHCYASWRNSSGTIELV |
| | (SEQ ID NO: 17) with 2 | KKGCWLDDFNCYDRQECVATEENPQVYFCCCEGNFCNERFTHLPEAGGPEV |
| | additional aa at N-terminus | TYEPPPTAPT |
| 473 | Hybrid hu-ActRIIB-ECD | GEAETRECIYYNANWEKDRTNQTGVEPCEGDQDKRLHCYASWRNSSGTIEL |
| | (SEQ ID NO: 17) with 3 | VKKGCWLDDFNCYDRQECVATEENPQVYFCCCEGNFCNERFTHLPEAGGPE |
| | additional aa at N-terminus | VTYEPPPTAPT |
| 474 | Hybrid hu-ActRIIB-ECD | RGEAETRECIYYNANWEKDRTNQTGVEPCEGDQDKRLHCYASWRNSSGTIE |
| | (SEQ ID NO: 17) with 4 | LVKKGCWLDDFNCYDRQECVATEENPQVYFCCCEGNFCNERFTHLPEAGGP |
| | additional aa at N-terminus | EVTYEPPPTAPT |
| 475 | Hybrid hu-ActRIIB-ECD | GRGEAETRECIYYNANWEKDRTNQTGVEPCEGDQDKRLHCYASWRNSSGTI |
| | (SEQ ID NO: 17) with 5 | ELVKKGCWLDDFNCYDRQECVATEENPQVYFCCCEGNFCNERFTHLPEAGG |
| | additional aa at N-terminus | PEVTYEPPPTAPT |
| 476 | Hybrid hu-ActRIIB-ECD | SGRGEAETRECIYYNANWEKDRTNQTGVEPCEGDQDKRLHCYASWRNSSG |
| | (SEQ ID NO: 17) with 6 | TIELVKKGCWLDDFNCYDRQECVATEENPQVYFCCCEGNFCNERFTHLPEA |
| | additional aa at N-terminus | GGPEVTYEPPPTAPT |
| 477 | Hybrid hu-ActRIIB-ECD | AETQECIYYNANWEKDRTNQTGVEPCEGDQDKRLHCYASWRNSSGTIELVK |
| | (SEQ ID NO: 18) with 1 | KGCWLDDFNCYDRQECVATEENPQVYFCCCEGNFCNERFTHLPEAGGPEVT |
| | additional aa at N-terminus | YEPPPTAPT |
| 478 | Hybrid hu-ActRIIB-ECD | EAETQECIYYNANWEKDRTNQTGVEPCEGDQDKRLHCYASWRNSSGTIELV |
| | (SEQ ID NO: 18) with 2 | KKGCWLDDFNCYDRQECVATEENPQVYFCCCEGNFCNERFTHLPEAGGPEV |
| | additional aa at N-terminus | TYEPPPTAPT |
| 479 | Hybrid hu-ActRIIB-ECD | GEAETQECIYYNANWEKDRTNQTGVEPCEGDQDKRLHCYASWRNSSGTIEL |
| | (SEQ ID NO: 18) with 3 | VKKGCWLDDFNCYDRQECVATEENPQVYFCCCEGNFCNERFTHLPEAGGPE |
| | additional aa at N-terminus | VTYEPPPTAPT |
| 480 | Hybrid hu-ActRIIB-ECD | RGEAETQECIYYNANWEKDRTNQTGVEPCEGDQDKRLHCYASWRNSSGTIE |
| | (SEQ ID NO: 18) with 4 | LVKKGCWLDDFNCYDRQECVATEENPQVYFCCCEGNFCNERFTHLPEAGGP |
| | additional aa at N-terminus | EVTYEPPPTAPT |
| 481 | Hybrid hu-ActRIIB-ECD | GRGEAETQECIYYNANWEKDRTNQTGVEPCEGDQDKRLHCYASWRNSSGTI |
| | (SEQ ID NO: 18) with 5 | ELVKKGCWLDDFNCYDRQECVATEENPQVYFCCCEGNFCNERFTHLPEAGG |
| | additional aa at N-terminus | PEVTYEPPPTAPT |
| 482 | Hybrid hu-ActRIIB-ECD | SGRGEAETQECIYYNANWEKDRTNQTGVEPCEGDQDKRLHCYASWRNSSG |
| | (SEQ ID NO: 18) with 6 | TIELVKKGCWLDDFNCYDRQECVATEENPQVYFCCCEGNFCNERFTHLPEA |
| | additional aa at N-terminus | GGPEVTYEPPPTAPT |
| 483 | Hybrid hu-ActRIIB-ECD | AETQECIYYNANWEKDRTNQTGLERCEGEQDKRLHCYASWRNSSGTIELVK |
| | (SEQ ID NO: 19) with 1 | KGCWLDDFNCYDRQECVATEENPQVYFCCCEGNFCNERFTHLPEAGGPEVT |
| | additional aa at N-terminus | YEPPPTAPT |
| 484 | Hybrid hu-ActRIIB-ECD | EAETQECIYYNANWEKDRTNQTGLERCEGEQDKRLHCYASWRNSSGTIELV |
| | (SEQ ID NO: 19) with 2 | KKGCWLDDFNCYDRQECVATEENPQVYFCCCEGNFCNERFTHLPEAGGPEV |
| | additional aa at N-terminus | TYEPPPTAPT |
| 485 | Hybrid hu-ActRIIB-ECD | GEAETQECIYYNANWEKDRTNQTGLERCEGEQDKRLHCYASWRNSSGTIEL |
| | (SEQ ID NO: 19) with 3 | VKKGCWLDDFNCYDRQECVATEENPQVYFCCCEGNFCNERFTHLPEAGGPE |
| | additional aa at N-terminus | VTYEPPPTAPT |
| 486 | Hybrid hu-ActRIIB-ECD | RGEAETQECIYYNANWEKDRTNQTGLERCEGEQDKRLHCYASWRNSSGTIE |
| | (SEQ ID NO: 19) with 4 | LVKKGCWLDDFNCYDRQECVATEENPQVYFCCCEGNFCNERFTHLPEAGGP |
| | additional aa at N-terminus | EVTYEPPPTAPT |
| 487 | Hybrid hu-ActRIIB-ECD | GRGEAETQECIYYNANWEKDRTNQTGLERCEGEQDKRLHCYASWRNSSGTI |
| | (SEQ ID NO: 19) with 5 | ELVKKGCWLDDFNCYDRQECVATEENPQVYFCCCEGNFCNERFTHLPEAGG |
| | additional aa at N-terminus | PEVTYEPPPTAPT |
| 488 | Hybrid hu-ActRIIB-ECD | SGRGEAETQECIYYNANWEKDRTNQTGLERCEGEQDKRLHCYASWRNSSG |
| | (SEQ ID NO: 19) with 6 | TIELVKKGCWLDDFNCYDRQECVATEENPQVYFCCCEGNFCNERFTHLPEA |
| | additional aa at N-terminus | GGPEVTYEPPPTAPT |
| 489 | Hybrid hu-ActRIIB-ECD | AETQECIYYNANWEKDRTNQSGLERCEGEQDKRLHCYASWRNSSGTIELVK |
| | (SEQ ID NO: 20) with 1 | KGCWLDDFNCYDRQECVATEENPQVYFCCCEGNFCNERFTHLPEAGGPEVT |
| | additional aa at N-terminus | YEPPPTAPT |
| 490 | Hybrid hu-ActRIIB-ECD | EAETQECIYYNANWEKDRTNQSGLERCEGEQDKRLHCYASWRNSSGTIELV |
| | (SEQ ID NO: 20) with 2 | KKGCWLDDFNCYDRQECVATEENPQVYFCCCEGNFCNERFTHLPEAGGPEV |
| | additional aa at N-terminus | TYEPPPTAPT |
| 491 | Hybrid hu-ActRIIB-ECD | GEAETQECIYYNANWEKDRTNQSGLERCEGEQDKRLHCYASWRNSSGTIEL |
| | (SEQ ID NO: 20) with 3 | VKKGCWLDDFNCYDRQECVATEENPQVYFCCCEGNFCNERFTHLPEAGGPE |
| | additional aa at N-terminus | VTYEPPPTAPT |
| 492 | Hybrid hu-ActRIIB-ECD | RGEAETQECIYYNANWEKDRTNQSGLERCEGEQDKRLHCYASWRNSSGTIE |
| | (SEQ ID NO: 20) with 4 | LVKKGCWLDDFNCYDRQECVATEENPQVYFCCCEGNFCNERFTHLPEAGGP |
| | additional aa at N-terminus | EVTYEPPPTAPT |
| 493 | Hybrid hu-ActRIIB-ECD | GRGEAETQECIYYNANWEKDRTNQSGLERCEGEQDKRLHCYASWRNSSGTI |
| | (SEQ ID NO: 20) with 5 | ELVKKGCWLDDFNCYDRQECVATEENPQVYFCCCEGNFCNERFTHLPEAGG |
| | additional aa at N-terminus | PEVTYEPPPTAPT |
| 494 | Hybrid hu-ActRIIB-ECD | SGRGEAETQECIYYNANWEKDRTNQSGLERCEGEQDKRLHCYASWRNSSG |
| | (SEQ ID NO: 20) with 6 | TIELVKKGCWLDDFNCYDRQECVATEENPQVYFCCCEGNFCNERFTHLPEA |
| | additional aa at N-terminus | GGPEVTYEPPPTAPT |
| 495 | Hybrid hu-ActRIIB-ECD | AETRECIYYNANWEKDRTNQSGLERCEGEQDKRLHCYASWRNSSGTIELVK |
| | (SEQ ID NO: 21) with 1 | KGCWLDDFNCYDRQECVATEENPQVYFCCCEGNFCNERFTHLPEAGGPEVT |
| | additional aa at N-terminus | YEPPPTAPT |
| 496 | Hybrid hu-ActRIIB-ECD | EAETRECIYYNANWEKDRTNQSGLERCEGEQDKRLHCYASWRNSSGTIELV |
| | (SEQ ID NO: 21) with 2 | KKGCWLDDFNCYDRQECVATEENPQVYFCCCEGNFCNERFTHLPEAGGPEV |
| | additional aa at N-terminus | TYEPPPTAPT |
| 497 | Hybrid hu-ActRIIB-ECD | GEAETRECIYYNANWEKDRTNQSGLERCEGEQDKRLHCYASWRNSSGTIEL |
| | (SEQ ID NO: 21) with 3 | VKKGCWLDDFNCYDRQECVATEENPQVYFCCCEGNFCNERFTHLPEAGGPE |
| | additional aa at N-terminus | VTYEPPPTAPT |
| 498 | Hybrid hu-ActRIIB-ECD | RGEAETRECIYYNANWEKDRTNQSGLERCEGEQDKRLHCYASWRNSSGTIE |
| | (SEQ ID NO: 21) with 4 | LVKKGCWLDDFNCYDRQECVATEENPQVYFCCCEGNFCNERFTHLPEAGGP |
| | additional aa at N-terminus | EVTYEPPPTAPT |
| 499 | Hybrid hu-ActRIIB-ECD | GRGEAETRECIYYNANWEKDRTNQSGLERCEGEQDKRLHCYASWRNSSGTI |
| | (SEQ ID NO: 21) with 5 | ELVKKGCWLDDFNCYDRQECVATEENPQVYFCCCEGNFCNERFTHLPEAGG |
| | additional aa at N-terminus | PEVTYEPPPTAPT |
| 500 | Hybrid hu-ActRIIB-ECD | SGRGEAETRECIYYNANWEKDRTNQSGLERCEGEQDKRLHCYASWRNSSGT |
| | (SEQ ID NO: 21) with 6 | IELVKKGCWLDDFNCYDRQECVATEENPQVYFCCCEGNFCNERFTHLPEAG |
| | additional aa at N-terminus | GPEVTYEPPPTAPT |
| 501 | Hybrid hu-ActRIIB-ECD | AETQECIYYNANWELERTNQSGLERCEGEQDKRLHCYASWRNSSGTIELVK |
| | (SEQ ID NO: 22) with 1 | KGCWLDDFNCYDRQECVATEENPQVYFCCCEGNFCNERFTHLPEAGGPEVT |
| | additional aa at N-terminus | YEPPPTAPT |
| 502 | Hybrid hu-ActRIIB-ECD | EAETQECIYYNANWELERTNQSGLERCEGEQDKRLHCYASWRNSSGTIELV |
| | (SEQ ID NO: 22) with 2 | KKGCWLDDFNCYDRQECVATEENPQVYFCCCEGNFCNERFTHLPEAGGPEV |
| | additional aa at N-terminus | TYEPPPTAPT |
| 503 | Hybrid hu-ActRIIB-ECD | GEAETQECIYYNANWELERTNQSGLERCEGEQDKRLHCYASWRNSSGTIEL |
| | (SEQ ID NO: 22) with 3 | VKKGCWLDDFNCYDRQECVATEENPQVYFCCCEGNFCNERFTHLPEAGGPE |
| | additional aa at N-terminus | VTYEPPPTAPT |
| 504 | Hybrid hu-ActRIIB-ECD | RGEAETQECIYYNANWELERTNQSGLERCEGEQDKRLHCYASWRNSSGTIE |
| | (SEQ ID NO: 22) with 4 | LVKKGCWLDDFNCYDRQECVATEENPQVYFCCCEGNFCNERFTHLPEAGGP |
| | additional aa at N-terminus | EVTYEPPPTAPT |
| 505 | Hybrid hu-ActRIIB-ECD | GRGEAETQECIYYNANWELERTNQSGLERCEGEQDKRLHCYASWRNSSGTI |
| | (SEQ ID NO: 22) with 5 | ELVKKGCWLDDFNCYDRQECVATEENPQVYFCCCEGNFCNERFTHLPEAGG |
| | additional aa at N-terminus | PEVTYEPPPTAPT |
| 506 | Hybrid hu-ActRIIB-ECD | SGRGEAETQECIYYNANWELERTNQSGLERCEGEQDKRLHCYASWRNSSGT |
| | (SEQ ID NO: 22) with 6 | IELVKKGCWLDDFNCYDRQECVATEENPQVYFCCCEGNFCNERFTHLPEAG |
| | additional aa at N-terminus | GPEVTYEPPPTAPT |
| 507 | Hybrid hu-ActRIIB-ECD | AETRECIYYNANWELERTNQSGLERCEGEQDKRLHCFATWRNSSGTIELVK |
| | (SEQ ID NO: 23) with 1 | QGCWLDDFNCYDRQECVATEENPQVYFCCCEGNFCNERFTHLPEAGGPEVT |
| | additional aa at N-terminus | YEPPPTAPT |
| 508 | Hybrid hu-ActRIIB-ECD | EAETRECIYYNANWELERTNQSGLERCEGEQDKRLHCFATWRNSSGTIELVK |
| | (SEQ ID NO: 23) with 2 | QGCWLDDFNCYDRQECVATEENPQVYFCCCEGNFCNERFTHLPEAGGPEVT |
| | additional aa at N-terminus | YEPPPTAPT |
| 509 | Hybrid hu-ActRIIB-ECD | GEAETRECIYYNANWELERTNQSGLERCEGEQDKRLHCFATWRNSSGTIELV |
| | (SEQ ID NO: 23) with 3 | KQGCWLDDFNCYDRQECVATEENPQVYFCCCEGNFCNERFTHLPEAGGPEV |
| | additional aa at N-terminus | TYEPPPTAPT |
| 510 | Hybrid hu-ActRIIB-ECD | RGEAETRECIYYNANWELERTNQSGLERCEGEQDKRLHCFATWRNSSGTIEL |
| | (SEQ ID NO: 23) with 4 | VKQGCWLDDFNCYDRQECVATEENPQVYFCCCEGNFCNERFTHLPEAGGPE |
| | additional aa at N-terminus | VTYEPPPTAPT |
| 511 | Hybrid hu-ActRIIB-ECD | GRGEAETRECIYYNANWELERTNQSGLERCEGEQDKRLHCFATWRNSSGTI |
| | (SEQ ID NO: 23) with 5 | ELVKQGCWLDDFNCYDRQECVATEENPQVYFCCCEGNFCNERFTHLPEAGG |
| | additional aa at N-terminus | PEVTYEPPPTAPT |
| 512 | Hybrid hu-ActRIIB-ECD | SGRGEAETRECIYYNANWELERTNQSGLERCEGEQDKRLHCFATWRNSSGTI |
| | (SEQ ID NO: 23) with 6 | ELVKQGCWLDDFNCYDRQECVATEENPQVYFCCCEGNFCNERFTHLPEAGG |
| | additional aa at N-terminus | PEVTYEPPPTAPT |
| 513 | Hybrid hu-ActRIIB-ECD | AETRECIYYNANWELERTNQSGLERCYGDKDKRRHCYASWRNSSGTIELVK |
| | (SEQ ID NO: 24) with 1 | KGCWLDDINCYDRQECVATEENPQVYFCCCEGNFCNERFTHLPEAGGPEVT |
| | additional aa at N-terminus | YEPPPTAPT |
| 514 | Hybrid hu-ActRIIB-ECD | EAETRECIYYNANWELERTNQSGLERCYGDKDKRRHCYASWRNSSGTIELV |
| | (SEQ ID NO: 24) with 2 | KKGCWLDDINCYDRQECVATEENPQVYFCCCEGNFCNERFTHLPEAGGPEV |
| | additional aa at N-terminus | TYEPPPTAPT |
| 515 | Hybrid hu-ActRIIB-ECD | GEAETRECIYYNANWELERTNQSGLERCYGDKDKRRHCYASWRNSSGTIEL |
| | (SEQ ID NO: 24) with 3 | VKKGCWLDDINCYDRQECVATEENPQVYFCCCEGNFCNERFTHLPEAGGPE |
| | additional aa at N-terminus | VTYEPPPTAPT |
| 516 | Hybrid hu-ActRIIB-ECD | RGEAETRECIYYNANWELERTNQSGLERCYGDKDKRRHCYASWRNSSGTIE |
| | (SEQ ID NO: 24) with 4 | LVKKGCWLDDINCYDRQECVATEENPQVYFCCCEGNFCNERFTHLPEAGGP |
| | additional aa at N-terminus | EVTYEPPPTAPT |
| 517 | Hybrid hu-ActRIIB-ECD | GRGEAETRECIYYNANWELERTNOSGLERCYGDKDKRRHCYASWRNSSGTI |
| | (SEQ ID NO: 24) with 5 | ELVKKGCWLDDINCYDRQECVATEENPQVYFCCCEGNFCNERFTHLPEAGG |
| | additional aa at N-terminus | PEVTYEPPPTAPT |
| 518 | Hybrid hu-ActRIIB-ECD | SGRGEAETRECIYYNANWELERTNQSGLERCYGDKDKRRHCYASWRNSSG |
| | (SEQ ID NO: 24) with 6 | TIELVKKGCWLDDINCYDRQECVATEENPQVYFCCCEGNFCNERFTHLPEAG |
| | additional aa at N-terminus | GPEVTYEPPPTAPT |
| 519 | Hybrid hu-ActRIIB-ECD | AETRECIYYNANWELERTNQSGLERCEGEQDKRLHCYASWRNSSGTIELVK |
| | (SEQ ID NO: 25) with 1 | KGCWLDDFNCYDRQECVATKENPQVYFCCCEGNFCNERFTHLPEAGGPEVT |
| | additional aa at N-terminus | YEPPPTAPT |
| 520 | Hybrid hu-ActRIIB-ECD | EAETRECIYYNANWELERTNQSGLERCEGEQDKRLHCYASWRNSSGTIELV |
| | (SEQ ID NO: 25) with 2 | KKGCWLDDFNCYDRQECVATKENPQVYFCCCEGNFCNERFTHLPEAGGPE |
| | additional aa at N-terminus | VTYEPPPTAPT |
| 521 | Hybrid hu-ActRIIB-ECD | GEAETRECIYYNANWELERTNQSGLERCEGEQDKRLHCYASWRNSSGTIEL |
| | (SEQ ID NO: 25) with 3 | VKKGCWLDDFNCYDRQECVATKENPQVYFCCCEGNFCNERFTHLPEAGGP |
| | additional aa at N-terminus | EVTYEPPPTAPT |
| 522 | Hybrid hu-ActRIIB-ECD | RGEAETRECIYYNANWELERTNQSGLERCEGEQDKRLHCYASWRNSSGTIE |
| | (SEQ ID NO: 25) with 4 | LVKKGCWLDDFNCYDRQECVATKENPQVYFCCCEGNFCNERFTHLPEAGG |
| | additional aa at N-terminus | PEVTYEPPPTAPT |
| 523 | Hybrid hu-ActRIIB-ECD | GRGEAETRECIYYNANWELERTNQSGLERCEGEQDKRLHCYASWRNSSGTI |
| | (SEQ ID NO: 25) with 5 | ELVKKGCWLDDFNCYDRQECVATKENPQVYFCCCEGNFCNERFTHLPEAG |
| | additional aa at N-terminus | GPEVTYEPPPTAPT |
| 524 | Hybrid hu-ActRIIB-ECD | SGRGEAETRECIYYNANWELERTNQSGLERCEGEQDKRLHCYASWRNSSGT |
| | (SEQ ID NO: 25) with 6 | IELVKKGCWLDDFNCYDRQECVATKENPQVYFCCCEGNFCNERFTHLPEAG |
| | additional aa at N-terminus | GPEVTYEPPPTAPT |
| 525 | Hybrid hu-ActRIIB-ECD | AETRECIYYNANWELERTNQSGLERCEGEQDKRLHCYASWRNSSGTIELVK |
| | (SEQ ID NO: 26) with 1 | KGCWLDDINCYDRQECVATEENPQVYFCCCEGNFCNERFTHLPEAGGPEVT |
| | additional aa at N-terminus | YEPPPTAPT |
| 526 | Hybrid hu-ActRIIB-ECD | EAETRECIYYNANWELERTNQSGLERCEGEQDKRLHCYASWRNSSGTIELV |
| | (SEQ ID NO: 26) with 2 | KKGCWLDDINCYDRQECVATEENPQVYFCCCEGNFCNERFTHLPEAGGPEV |
| | additional aa at N-terminus | TYEPPPTAPT |
| 527 | Hybrid hu-ActRIIB-ECD | GEAETRECIYYNANWELERTNQSGLERCEGEQDKRLHCYASWRNSSGTIEL |
| | (SEQ ID NO: 26) with 3 | VKKGCWLDDINCYDRQECVATEENPQVYFCCCEGNFCNERFTHLPEAGGPE |
| | additional aa at N-terminus | VTYEPPPTAPT |
| 528 | Hybrid hu-ActRIIB-ECD | RGEAETRECIYYNANWELERTNQSGLERCEGEQDKRLHCYASWRNSSGTIE |
| | (SEQ ID NO: 26) with 4 | LVKKGCWLDDINCYDRQECVATEENPQVYFCCCEGNFCNERFTHLPEAGGP |
| | additional aa at N-terminus | EVTYEPPPTAPT |
| 529 | Hybrid hu-ActRIIB-ECD | GRGEAETRECIYYNANWELERTNQSGLERCEGEQDKRLHCYASWRNSSGTI |
| | (SEQ ID NO: 26) with 5 | ELVKKGCWLDDINCYDRQECVATEENPQVYFCCCEGNFCNERFTHLPEAGG |
| | additional aa at N-terminus | PEVTYEPPPTAPT |
| 530 | Hybrid hu-ActRIIB-ECD | SGRGEAETRECIYYNANWELERTNQSGLERCEGEQDKRLHCYASWRNSSGT |
| | (SEQ ID NO: 26) with 6 | IELVKKGCWLDDINCYDRQECVATEENPQVYFCCCEGNFCNERFTHLPEAG |
| | additional aa at N-terminus | GPEVTYEPPPTAPT |
| 531 | Hybrid hu-ActRIIB-ECD | AETRECIYYNANWELERTNQSGLERCEGEQDKRLHCYASWRNSSGTIELVK |
| | (SEQ ID NO: 27) with 1 | KGCWLDDINCYDRQECVATKENPQVYFCCCEGNFCNERFTHLPEAGGPEVT |
| | additional aa at N-terminus | YEPPPTAPT |
| 532 | Hybrid hu-ActRIIB-ECD | EAETRECIYYNANWELERTNQSGLERCEGEQDKRLHCYASWRNSSGTIELV |
| | (SEQ ID NO: 27) with 2 | KKGCWLDDINCYDRQECVATKENPQVYFCCCEGNFCNERFTHLPEAGGPEV |
| | additional aa at N-terminus | TYEPPPTAPT |
| 533 | Hybrid hu-ActRIIB-ECD | GEAETRECIYYNANWELERTNQSGLERCEGEQDKRLHCYASWRNSSGTIEL |
| | (SEQ ID NO: 27) with 3 | VKKGCWLDDINCYDRQECVATKENPQVYFCCCEGNFCNERFTHLPEAGGPE |
| | additional aa at N-terminus | VTYEPPPTAPT |
| 534 | Hybrid hu-ActRIIB-ECD | RGEAETRECIYYNANWELERTNQSGLERCEGEQDKRLHCYASWRNSSGTIE |
| | (SEQ ID NO: 27) with 4 | LVKKGCWLDDINCYDRQECVATKENPQVYFCCCEGNFCNERFTHLPEAGGP |
| | additional aa at N-terminus | EVTYEPPPTAPT |
| 535 | Hybrid hu-ActRIIB-ECD | GRGEAETRECIYYNANWELERTNQSGLERCEGEQDKRLHCYASWRNSSGTI |
| | (SEQ ID NO: 27) with 5 | ELVKKGCWLDDINCYDRQECVATKENPQVYFCCCEGNFCNERFTHLPEAGG |
| | additional aa at N-terminus | PEVTYEPPPTAPT |
| 536 | Hybrid hu-ActRIIB-ECD | SGRGEAETRECIYYNANWELERTNQSGLERCEGEQDKRLHCYASWRNSSGT |
| | (SEQ ID NO: 27) with 6 | IELVKKGCWLDDINCYDRQECVATKENPQVYFCCCEGNFCNERFTHLPEAG |
| | additional aa at N-terminus | GPEVTYEPPPTAPT |
| 537 | Hybrid hu-ActRIIB-ECD | AETRECIYYNANWELERTNQSGLERCEGDKDKRLHCYASWRNSSGTIELVK |
| | (SEQ ID NO: 28) with 1 | KGCWLDDINCYDRQECVATKENPQVYFCCCEGNFCNERFTHLPEAGGPEVT |
| | additional aa at N-terminus | YEPPPTAPT |
| 538 | Hybrid hu-ActRIIB-ECD | EAETRECIYYNANWELERTNQSGLERCEGDKDKRLHCYASWRNSSGTIELV |
| | (SEQ ID NO: 28) with 2 | KKGCWLDDINCYDRQECVATKENPQVYFCCCEGNFCNERFTHLPEAGGPEV |
| | additional aa at N-terminus | TYEPPPTAPT |
| 539 | Hybrid hu-ActRIIB-ECD | GEAETRECIYYNANWELERTNQSGLERCEGDKDKRLHCYASWRNSSGTIEL |
| | (SEQ ID NO: 28) with 3 | VKKGCWLDDINCYDRQECVATKENPQVYFCCCEGNFCNERFTHLPEAGGPE |
| | additional aa at N-terminus | VTYEPPPTAPT |
| 540 | Hybrid hu-ActRIIB-ECD | RGEAETRECIYYNANWELERTNQSGLERCEGDKDKRLHCYASWRNSSGTIE |
| | (SEQ ID NO: 28) with 4 | LVKKGCWLDDINCYDRQECVATKENPQVYFCCCEGNFCNERFTHLPEAGGP |
| | additional aa at N-terminus | EVTYEPPPTAPT |
| 541 | Hybrid hu-ActRIIB-ECD | GRGEAETRECIYYNANWELERTNQSGLERCEGDKDKRLHCYASWRNSSGTI |
| | (SEQ ID NO: 28) with 5 | ELVKKGCWLDDINCYDRQECVATKENPQVYFCCCEGNFCNERFTHLPEAGG |
| | additional aa at N-terminus | PEVTYEPPPTAPT |
| 542 | Hybrid hu-ActRIIB-ECD | SGRGEAETRECIYYNANWELERTNQSGLERCEGDKDKRLHCYASWRNSSGT |
| | (SEQ ID NO: 28) with 6 | IELVKKGCWLDDINCYDRQECVATKENPQVYFCCCEGNFCNERFTHLPEAG |
| | additional aa at N-terminus | GPEVTYEPPPTAPT |
| 543 | Hybrid hu-ActRIIB-ECD | AETRECIYYNANWELERTNQSGLERCEGDQDKRLHCYASWRNSSGTIELVK |
| | (SEQ ID NO: 29) with 1 | KGCWLDDINCYDRQECVATKENPQVYFCCCEGNFCNERFTHLPEAGGPEVT |
| | additional aa at N-terminus | YEPPPTAPT |
| 544 | Hybrid hu-ActRIIB-ECD | EAETRECIYYNANWELERTNQSGLERCEGDQDKRLHCYASWRNSSGTIELV |
| | (SEQ ID NO: 29) with 2 | KKGCWLDDINCYDRQECVATKENPQVYFCCCEGNFCNERFTHLPEAGGPEV |
| | additional aa at N-terminus | TYEPPPTAPT |
| 545 | Hybrid hu-ActRIIB-ECD | GEAETRECIYYNANWELERTNQSGLERCEGDQDKRLHCYASWRNSSGTIEL |
| | (SEQ ID NO: 29) with 3 | VKKGCWLDDINCYDRQECVATKENPQVYFCCCEGNFCNERFTHLPEAGGPE |
| | additional aa at N-terminus | VTYEPPPTAPT |
| 546 | Hybrid hu-ActRIIB-ECD | RGEAETRECIYYNANWELERTNQSGLERCEGDQDKRLHCYASWRNSSGTIE |
| | (SEQ ID NO: 29) with 4 | LVKKGCWLDDINCYDRQECVATKENPQVYFCCCEGNFCNERFTHLPEAGGP |
| | additional aa at N-terminus | EVTYEPPPTAPT |
| 547 | Hybrid hu-ActRIIB-ECD | GRGEAETRECIYYNANWELERTNQSGLERCEGDQDKRLHCYASWRNSSGTI |
| | (SEQ ID NO: 29) with 5 | ELVKKGCWLDDINCYDRQECVATKENPQVYFCCCEGNFCNERFTHLPEAGG |
| | additional aa at N-terminus | PEVTYEPPPTAPT |
| 548 | Hybrid hu-ActRIIB-ECD | SGRGEAETRECIYYNANWELERTNQSGLERCEGDQDKRLHCYASWRNSSGT |
| | (SEQ ID NO: 29) with 6 | IELVKKGCWLDDINCYDRQECVATKENPQVYFCCCEGNFCNERFTHLPEAG |
| | additional aa at N-terminus | GPEVTYEPPPTAPT |
| 549 | Hybrid hu-ActRIIB-ECD | AETRECIYYNANWELERTNQSGLERCEGDQDKRLHCYASWRNSSGTIELVK |
| | (SEQ ID NO: 30) with 1 | KGCWLDDFNCYDRQECVATKENPQVYFCCCEGNFCNERFTHLPEAGGPEVT |
| | additional aa at N-terminus | YEPPPTAPT |
| 550 | Hybrid hu-ActRIIB-ECD | EAETRECIYYNANWELERTNQSGLERCEGDQDKRLHCYASWRNSSGTIELV |
| | (SEQ ID NO: 30) with 2 | KKGCWLDDFNCYDRQECVATKENPQVYFCCCEGNFCNERFTHLPEAGGPE |
| | additional aa at N-terminus | VTYEPPPTAPT |
| 551 | Hybrid hu-ActRIIB-ECD | GEAETRECIYYNANWELERTNQSGLERCEGDQDKRLHCYASWRNSSGTIEL |
| | (SEQ ID NO: 30) with 3 | VKKGCWLDDFNCYDRQECVATKENPQVYFCCCEGNFCNERFTHLPEAGGP |
| | additional aa at N-terminus | EVTYEPPPTAPT |
| 552 | Hybrid hu-ActRIIB-ECD | RGEAETRECIYYNANWELERTNQSGLERCEGDQDKRLHCYASWRNSSGTIE |
| | (SEQ ID NO: 30) with 4 | LVKKGCWLDDFNCYDRQECVATKENPQVYFCCCEGNFCNERFTHLPEAGG |
| | additional aa at N-terminus | PEVTYEPPPTAPT |
| 553 | Hybrid hu-ActRIIB-ECD | GRGEAETRECIYYNANWELERTNQSGLERCEGDQDKRLHCYASWRNSSGTI |
| | (SEQ ID NO: 30) with 5 | ELVKKGCWLDDFNCYDRQECVATKENPQVYFCCCEGNFCNERFTHLPEAG |
| | additional aa at N-terminus | GPEVTYEPPPTAPT |
| 554 | Hybrid hu-ActRIIB-ECD | SGRGEAETRECIYYNANWELERTNQSGLERCEGDQDKRLHCYASWRNSSGT |
| | (SEQ ID NO: 30) with 6 | IELVKKGCWLDDFNCYDRQECVATKENPQVYFCCCEGNFCNERFTHLPEAG |
| | additional aa at N-terminus | GPEVTYEPPPTAPT |
| 555 | Hybrid hu-ActRIIB-ECD | AETRECIYYNANWELERTNQSGLERCEGDQDKRLHCYASWRNSSGTIELVK |
| | (SEQ ID NO: 31) with 1 | KGCWLDDFNCYDRQECVATEENPQVYFCCCEGNFCNERFTHLPEAGGPEVT |
| | additional aa at N-terminus | YEPPPTAPT |
| 556 | Hybrid hu-ActRIIB-ECD | EAETRECIYYNANWELERTNQSGLERCEGDQDKRLHCYASWRNSSGTIELV |
| | (SEQ ID NO: 31) with 2 | KKGCWLDDFNCYDRQECVATEENPQVYFCCCEGNFCNERFTHLPEAGGPEV |
| | additional aa at N-terminus | TYEPPPTAPT |
| 557 | Hybrid hu-ActRIIB-ECD | GEAETRECIYYNANWELERTNQSGLERCEGDQDKRLHCYASWRNSSGTIEL |
| | (SEQ ID NO: 31) with 3 | VKKGCWLDDFNCYDRQECVATEENPQVYFCCCEGNFCNERFTHLPEAGGPE |
| | additional aa at N-terminus | VTYEPPPTAPT |
| 558 | Hybrid hu-ActRIIB-ECD | RGEAETRECIYYNANWELERTNQSGLERCEGDQDKRLHCYASWRNSSGTIE |
| | (SEQ ID NO: 31) with 4 | LVKKGCWLDDFNCYDRQECVATEENPQVYFCCCEGNFCNERFTHLPEAGGP |
| | additional aa at N-terminus | EVTYEPPPTAPT |
| 559 | Hybrid hu-ActRIIB-ECD | GRGEAETRECIYYNANWELERTNQSGLERCEGDQDKRLHCYASWRNSSGTI |
| | (SEQ ID NO: 31) with 5 | ELVKKGCWLDDFNCYDRQECVATEENPQVYFCCCEGNFCNERFTHLPEAGG |
| | additional aa at N-terminus | PEVTYEPPPTAPT |
| 560 | Hybrid hu-ActRIIB-ECD | SGRGEAETRECIYYNANWELERTNQSGLERCEGDQDKRLHCYASWRNSSGT |
| | (SEQ ID NO: 31) with 6 | IELVKKGCWLDDFNCYDRQECVATEENPQVYFCCCEGNFCNERFTHLPEAG |
| | additional aa at N-terminus | GPEVTYEPPPTAPT |
| 561 | Hybrid hu-ActRIIB-ECD | AETRECIYYNANWELERTNQSGLERCYGDKDKRRHCYASWRNSSGTIELVK |
| | (SEQ ID NO: 32) with 1 | KGCWLDDINCYDRQECVEKKDSPEVYFCCCEGNFCNERFTHLPEAGGPEVT |
| | additional aa at N-terminus | YEPPPTAPT |
| 562 | Hybrid hu-ActRIIB-ECD | EAETRECIYYNANWELERTNQSGLERCYGDKDKRRHCYASWRNSSGTIELV |
| | (SEQ ID NO: 32) with 2 | KKGCWLDDINCYDRQECVEKKDSPEVYFCCCEGNFCNERFTHLPEAGGPEV |
| | additional aa at N-terminus | TYEPPPTAPT |
| 563 | Hybrid hu-ActRIIB-ECD | GEAETRECIYYNANWELERTNQSGLERCYGDKDKRRHCYASWRNSSGTIEL |
| | (SEQ ID NO: 32) with 3 | VKKGCWLDDINCYDRQECVEKKDSPEVYFCCCEGNFCNERFTHLPEAGGPE |
| | additional aa at N-terminus | VTYEPPPTAPT |
| 564 | Hybrid hu-ActRIIB-ECD | RGEAETRECIYYNANWELERTNQSGLERCYGDKDKRRHCYASWRNSSGTIE |
| | (SEQ ID NO: 32) with 4 | LVKKGCWLDDINCYDRQECVEKKDSPEVYFCCCEGNFCNERFTHLPEAGGP |
| | additional aa at N-terminus | EVTYEPPPTAPT |
| 565 | Hybrid hu-ActRIIB-ECD | GRGEAETRECIYYNANWELERTNQSGLERCYGDKDKRRHCYASWRNSSGTI |
| | (SEQ ID NO: 32) with 5 | ELVKKGCWLDDINCYDRQECVEKKDSPEVYFCCCEGNFCNERFTHLPEAGG |
| | additional aa at N-terminus | PEVTYEPPPTAPT |
| 566 | Hybrid hu-ActRIIB-ECD | SGRGEAETRECIYYNANWELERTNQSGLERCYGDKDKRRHCYASWRNSSG |
| | (SEQ ID NO: 32) with 6 | TIELVKKGCWLDDINCYDRQECVEKKDSPEVYFCCCEGNFCNERFTHLPEAG |
| | additional aa at N-terminus | GPEVTYEPPPTAPT |
| 567 | Hybrid hu-ActRIIB-ECD | AETRECIFFNANWEKDRTNQTGVEPCEGEQDKRLHCYASWRNSSGTIELVK |
| | (SEQ ID NO: 33) with 1 | KGCWLDDFNCYDRQECVATEENPQVYFCCCEGNFCNERFTHLPEAGGPEVT |
| | additional aa at N-terminus | YEPPPTAPT |
| 568 | Hybrid hu-ActRIIB-ECD | EAETRECIFFNANWEKDRTNQTGVEPCEGEQDKRLHCYASWRNSSGTIELV |
| | (SEQ ID NO: 33) with 2 | KKGCWLDDFNCYDRQECVATEENPQVYFCCCEGNFCNERFTHLPEAGGPEV |
| | additional aa at N-terminus | TYEPPPTAPT |
| 569 | Hybrid hu-ActRIIB-ECD | GEAETRECIFFNANWEKDRTNQTGVEPCEGEQDKRLHCYASWRNSSGTIEL |
| | (SEQ ID NO: 33) with 3 | VKKGCWLDDFNCYDRQECVATEENPQVYFCCCEGNFCNERFTHLPEAGGPE |
| | additional aa at N-terminus | VTYEPPPTAPT |
| 570 | Hybrid hu-ActRIIB-ECD | RGEAETRECIFFNANWEKDRTNQTGVEPCEGEQDKRLHCYASWRNSSGTIEL |
| | (SEQ ID NO: 33) with 4 | VKKGCWLDDFNCYDRQECVATEENPQVYFCCCEGNFCNERFTHLPEAGGPE |
| | additional aa at N-terminus | VTYEPPPTAPT |
| 571 | Hybrid hu-ActRIIB-ECD | GRGEAETRECIFFNANWEKDRTNQTGVEPCEGEQDKRLHCYASWRNSSGTI |
| | (SEQ ID NO: 33) with 5 | ELVKKGCWLDDFNCYDRQECVATEENPQVYFCCCEGNFCNERFTHLPEAGG |
| | additional aa at N-terminus | PEVTYEPPPTAPT |
| 572 | Hybrid hu-ActRIIB-ECD | SGRGEAETRECIFFNANWEKDRTNQTGVEPCEGEQDKRLHCYASWRNSSGT |
| | (SEQ ID NO: 33) with 6 | IELVKKGCWLDDFNCYDRQECVATEENPQVYFCCCEGNFCNERFTHLPEAG |
| | additional aa at N-terminus | GPEVTYEPPPTAPT |
| 573 | Hybrid hu-ActRIIB-ECD | AETRECIYYNANWELERTNQSGLERCEGEQDKRLHCFATWKNISGSIELVKQ |
| | (SEQ ID NO: 34) with 1 | GCWLDDFNCYDRQECVATEENPQVYFCCCEGNFCNERFTHLPEAGGPEVTY |
| | additional aa at N-terminus | EPPPTAPT |
| 574 | Hybrid hu-ActRIIB-ECD | EAETRECIYYNANWELERTNQSGLERCEGEQDKRLHCFATWKNISGSIELVK |
| | (SEQ ID NO: 34) with 2 | QGCWLDDFNCYDRQECVATEENPQVYFCCCEGNFCNERFTHLPEAGGPEVT |
| | additional aa at N-terminus | YEPPPTAPT |
| 575 | Hybrid hu-ActRIIB-ECD | GEAETRECIYYNANWELERTNQSGLERCEGEQDKRLHCFATWKNISGSIELV |
| | (SEQ ID NO: 34) with 3 | KQGCWLDDFNCYDRQECVATEENPQVYFCCCEGNFCNERFTHLPEAGGPEV |
| | additional aa at N-terminus | TYEPPPTAPT |
| 576 | Hybrid hu-ActRIIB-ECD | RGEAETRECIYYNANWELERTNQSGLERCEGEQDKRLHCFATWKNISGSIEL |
| | (SEQ ID NO: 34) with 4 | VKQGCWLDDFNCYDRQECVATEENPQVYFCCCEGNFCNERFTHLPEAGGPE |
| | additional aa at N-terminus | VTYEPPPTAPT |
| 577 | Hybrid hu-ActRIIB-ECD | GRGEAETRECIYYNANWELERTNQSGLERCEGEQDKRLHCFATWKNISGSIE |
| | (SEQ ID NO: 34) with 5 | LVKQGCWLDDFNCYDRQECVATEENPQVYFCCCEGNFCNERFTHLPEAGGP |
| | additional aa at N-terminus | EVTYEPPPTAPT |
| 578 | Hybrid hu-ActRIIB-ECD | SGRGEAETRECIYYNANWELERTNQSGLERCEGEQDKRLHCFATWKNISGSI |
| | (SEQ ID NO: 34) with 6 | ELVKQGCWLDDFNCYDRQECVATEENPQVYFCCCEGNFCNERFTHLPEAGG |
| | additional aa at N-terminus | PEVTYEPPPTAPT |
| 579 | Hybrid hu-ActRIIB-ECD | AETRECIYYNANWELERTNQSGLERCEGEQDKRLHCYASWRNSSGTIELVK |
| | (SEQ ID NO: 35) with 1 | KGCWLDDFNCYDRTDCVATEENPQVYFCCCEGNFCNERFTHLPEAGGPEVT |
| | additional aa at N-terminus | YEPPPTAPT |
| 580 | Hybrid hu-ActRIIB-ECD | EAETRECIYYNANWELERTNQSGLERCEGEQDKRLHCYASWRNSSGTIELV |
| | (SEQ ID NO: 35) with 2 | KKGCWLDDFNCYDRTDCVATEENPQVYFCCCEGNFCNERFTHLPEAGGPEV |
| | additional aa at N-terminus | TYEPPPTAPT |
| 581 | Hybrid hu-ActRIIB-ECD | GEAETRECIYYNANWELERTNQSGLERCEGEQDKRLHCYASWRNSSGTIEL |
| | (SEQ ID NO: 35) with 3 | VKKGCWLDDFNCYDRTDCVATEENPQVYFCCCEGNFCNERFTHLPEAGGPE |
| | additional aa at N-terminus | VTYEPPPTAPT |
| 582 | Hybrid hu-ActRIIB-ECD | RGEAETRECIYYNANWELERTNQSGLERCEGEQDKRLHCYASWRNSSGTIE |
| | (SEQ ID NO: 35) with 4 | LVKKGCWLDDFNCYDRTDCVATEENPQVYFCCCEGNFCNERFTHLPEAGGP |
| | additional aa at N-terminus | EVTYEPPPTAPT |
| 583 | Hybrid hu-ActRIIB-ECD | GRGEAETRECIYYNANWELERTNQSGLERCEGEQDKRLHCYASWRNSSGTI |
| | (SEQ ID NO: 35) with 5 | ELVKKGCWLDDFNCYDRTDCVATEENPQVYFCCCEGNFCNERFTHLPEAGG |
| | additional aa at N-terminus | PEVTYEPPPTAPT |
| 584 | Hybrid hu-ActRIIB-ECD | SGRGEAETRECIYYNANWELERTNQSGLERCEGEQDKRLHCYASWRNSSGT |
| | (SEQ ID NO: 35) with 6 | IELVKKGCWLDDFNCYDRTDCVATEENPQVYFCCCEGNFCNERFTHLPEAG |
| | additional aa at N-terminus | GPEVTYEPPPTAPT |
| 585 | Hybrid hu-ActRIIB-ECD | AETRECIYYNANWELERTNQSGLERCEGEQDKRLHCYASWRNSSGTIELVK |
| | (SEQ ID NO: 36) with 1 | KGCWLDDFNCYDRQECVATEENPQVYFCCCEGNMCNERFTHLPEAGGPEV |
| | additional aa at N-terminus | TYEPPPTAPT |
| 586 | Hybrid hu-ActRIIB-ECD | EAETRECIYYNANWELERTNQSGLERCEGEQDKRLHCYASWRNSSGTIELV |
| | (SEQ ID NO: 36) with 2 | KKGCWLDDFNCYDRQECVATEENPQVYFCCCEGNMCNERFTHLPEAGGPE |
| | additional aa at N-terminus | VTYEPPPTAPT |
| 587 | Hybrid hu-ActRIIB-ECD | GEAETRECIYYNANWELERTNQSGLERCEGEQDKRLHCYASWRNSSGTIEL |
| | (SEQ ID NO: 36) with 3 | VKKGCWLDDFNCYDRQECVATEENPQVYFCCCEGNMCNERFTHLPEAGGP |
| | additional aa at N-terminus | EVTYEPPPTAPT |
| 588 | Hybrid hu-ActRIIB-ECD | RGEAETRECIYYNANWELERTNQSGLERCEGEQDKRLHCYASWRNSSGTIE |
| | (SEQ ID NO: 36) with 4 | LVKKGCWLDDFNCYDRQECVATEENPQVYFCCCEGNMCNERFTHLPEAGG |
| | additional aa at N-terminus | PEVTYEPPPTAPT |
| 589 | Hybrid hu-ActRIIB-ECD | GRGEAETRECIYYNANWELERTNQSGLERCEGEQDKRLHCYASWRNSSGTI |
| | (SEQ ID NO: 36) with 5 | ELVKKGCWLDDFNCYDRQECVATEENPQVYFCCCEGNMCNERFTHLPEAG |
| | additional aa at N-terminus | GPEVTYEPPPTAPT |
| 590 | Hybrid hu-ActRIIB-ECD | SGRGEAETRECIYYNANWELERTNQSGLERCEGEQDKRLHCYASWRNSSGT |
| | (SEQ ID NO: 36) with 6 | IELVKKGCWLDDFNCYDRQECVATEENPQVYFCCCEGNMCNERFTHLPEAG |
| | additional aa at N-terminus | GPEVTYEPPPTAPT |
| 591 | Hybrid hu-ActRIIB-ECD | AETRECIYYNANWELERTNQSGLERCEGDQDKRLHCYASWRNSSGTIELVK |
| | (SEQ ID NO: 37) with 1 | KGCWLDDINCYDRQECVATEENPQVYFCCCEGNFCNERFTHLPEAGGPEVT |
| | additional aa at N-terminus | YEPPPTAPT |
| 592 | Hybrid hu-ActRIIB-ECD | EAETRECIYYNANWELERTNQSGLERCEGDQDKRLHCYASWRNSSGTIELV |
| | (SEQ ID NO: 37) with 2 | KKGCWLDDINCYDRQECVATEENPQVYFCCCEGNFCNERFTHLPEAGGPEV |
| | additional aa at N-terminus | TYEPPPTAPT |
| 593 | Hybrid hu-ActRIIB-ECD | GEAETRECIYYNANWELERTNQSGLERCEGDQDKRLHCYASWRNSSGTIEL |
| | (SEQ ID NO: 37) with 3 | VKKGCWLDDINCYDRQECVATEENPQVYFCCCEGNFCNERFTHLPEAGGPE |
| | additional aa at N-terminus | VTYEPPPTAPT |
| 594 | Hybrid hu-ActRIIB-ECD | RGEAETRECIYYNANWELERTNQSGLERCEGDQDKRLHCYASWRNSSGTIE |
| | (SEQ ID NO: 37) with 4 | LVKKGCWLDDINCYDRQECVATEENPQVYFCCCEGNFCNERFTHLPEAGGP |
| | additional aa at N-terminus | EVTYEPPPTAPT |
| 595 | Hybrid hu-ActRIIB-ECD | GRGEAETRECIYYNANWELERTNQSGLERCEGDQDKRLHCYASWRNSSGTI |
| | (SEQ ID NO: 37) with 5 | ELVKKGCWLDDINCYDRQECVATEENPQVYFCCCEGNFCNERFTHLPEAGG |
| | additional aa at N-terminus | PEVTYEPPPTAPT |
| 596 | Hybrid hu-ActRIIB-ECD | SGRGEAETRECIYYNANWELERTNQSGLERCEGDQDKRLHCYASWRNSSGT |
| | (SEQ ID NO: 37) with 6 | IELVKKGCWLDDINCYDRQECVATEENPQVYFCCCEGNFCNERFTHLPEAG |
| | additional aa at N-terminus | GPEVTYEPPPTAPT |
| 597 | Hybrid hu-ActRIIB-ECD | AETQECLFFNANWEKDRTNQSGVEPCYGDKDKRRHCYASWRNSSGTIELVK |
| | (SEQ ID NO: 51) with 1 | KGCWLDDFNCYDRQECVATEENPQVYFCCCEGNFCNERFTHLPEAGGPEVT |
| | additional aa at N-terminus | YEPPPTAPT |
| 598 | Hybrid hu-ActRIIB-ECD | EAETQECLFFNANWEKDRTNQSGVEPCYGDKDKRRHCYASWRNSSGTIELV |
| | (SEQ ID NO: 51) with 2 | KKGCWLDDFNCYDRQECVATEENPQVYFCCCEGNFCNERFTHLPEAGGPEV |
| | additional aa at N-terminus | TYEPPPTAPT |
| 599 | Hybrid hu-ActRIIB-ECD | GEAETQECLFFNANWEKDRTNQSGVEPCYGDKDKRRHCYASWRNSSGTIEL |
| | (SEQ ID NO: 51) with 3 | VKKGCWLDDFNCYDRQECVATEENPQVYFCCCEGNFCNERFTHLPEAGGPE |
| | additional aa at N-terminus | VTYEPPPTAPT |
| 600 | Hybrid hu-ActRIIB-ECD | RGEAETQECLFFNANWEKDRTNQSGVEPCYGDKDKRRHCYASWRNSSGTIE |
| | (SEQ ID NO: 51) with 4 | LVKKGCWLDDFNCYDRQECVATEENPQVYFCCCEGNFCNERFTHLPEAGGP |
| | additional aa at N-terminus | EVTYEPPPTAPT |
| 601 | Hybrid hu-ActRIIB-ECD | GRGEAETQECLFFNANWEKDRTNQSGVEPCYGDKDKRRHCYASWRNSSGT |
| | (SEQ ID NO: 51) with 5 | IELVKKGCWLDDFNCYDRQECVATEENPQVYFCCCEGNFCNERFTHLPEAG |
| | additional aa at N-terminus | GPEVTYEPPPTAPT |
| 602 | Hybrid hu-ActRIIB-ECD | SGRGEAETQECLFFNANWEKDRTNQSGVEPCYGDKDKRRHCYASWRNSSG |
| | (SEQ ID NO: 51) with 6 | TIELVKKGCWLDDFNCYDRQECVATEENPQVYFCCCEGNFCNERFTHLPEA |
| | additional aa at N-terminus | GGPEVTYEPPPTAPT |
| 603 | Hybrid hu-ActRIIB-ECD | AETQECLFFNANWEKDRTNQSGVEPCEGEQDKRLHCYASWRNSSGTIELVK |
| | (SEQ ID NO: 52) with 1 | KGCWLDDFNCYDRQECVATEENPQVYFCCCEGNFCNERFTHLPEAGGPEVT |
| | additional aa at N-terminus | YEPPPTAPT |
| 604 | Hybrid hu-ActRIIB-ECD | EAETQECLFFNANWEKDRTNQSGVEPCEGEQDKRLHCYASWRNSSGTIELV |
| | (SEQ ID NO: 52) with 2 | KKGCWLDDFNCYDRQECVATEENPQVYFCCCEGNFCNERFTHLPEAGGPEV |
| | additional aa at N-terminus | TYEPPPTAPT |
| 605 | Hybrid hu-ActRIIB-ECD | GEAETQECLFFNANWEKDRTNQSGVEPCEGEQDKRLHCYASWRNSSGTIEL |
| | (SEQ ID NO: 52) with 3 | VKKGCWLDDFNCYDRQECVATEENPQVYFCCCEGNFCNERFTHLPEAGGPE |
| | additional aa at N-terminus | VTYEPPPTAPT |
| 606 | Hybrid hu-ActRIIB-ECD | RGEAETQECLFFNANWEKDRTNQSGVEPCEGEQDKRLHCYASWRNSSGTIE |
| | (SEQ ID NO: 52) with 4 | LVKKGCWLDDFNCYDRQECVATEENPQVYFCCCEGNFCNERFTHLPEAGGP |
| | additional aa at N-terminus | EVTYEPPPTAPT |
| 607 | Hybrid hu-ActRIIB-ECD | GRGEAETQECLFFNANWEKDRTNQSGVEPCEGEQDKRLHCYASWRNSSGTI |
| | (SEQ ID NO: 52) with 5 | ELVKKGCWLDDFNCYDRQECVATEENPQVYFCCCEGNFCNERFTHLPEAGG |
| | additional aa at N-terminus | PEVTYEPPPTAPT |
| 608 | Hybrid hu-ActRIIB-ECD | SGRGEAETQECLFFNANWEKDRTNQSGVEPCEGEQDKRLHCYASWRNSSGT |
| | (SEQ ID NO: 52) with 6 | IELVKKGCWLDDFNCYDRQECVATEENPQVYFCCCEGNFCNERFTHLPEAG |
| | additional aa at N-terminus | GPEVTYEPPPTAPT |
| 609 | Hybrid hu-ActRIIB-ECD | AETRECLFFNANWEKDRTNQSGVEPCEGEQDKRLHCYASWRNSSGTIELVK |
| | (SEQ ID NO: 53) with 1 | KGCWLDDFNCYDRQECVATEENPQVYFCCCEGNFCNERFTHLPEAGGPEVT |
| | additional aa at N-terminus | YEPPPTAPT |
| 610 | Hybrid hu-ActRIIB-ECD | EAETRECLFFNANWEKDRTNQSGVEPCEGEQDKRLHCYASWRNSSGTIELV |
| | (SEQ ID NO: 53) with 2 | KKGCWLDDFNCYDRQECVATEENPQVYFCCCEGNFCNERFTHLPEAGGPEV |
| | additional aa at N-terminus | TYEPPPTAPT |
| 611 | Hybrid hu-ActRIIB-ECD | GEAETRECLFFNANWEKDRTNQSGVEPCEGEQDKRLHCYASWRNSSGTIEL |
| | (SEQ ID NO: 53) with 3 | VKKGCWLDDFNCYDRQECVATEENPQVYFCCCEGNFCNERFTHLPEAGGPE |
| | additional aa at N-terminus | VTYEPPPTAPT |
| 612 | Hybrid hu-ActRIIB-ECD | RGEAETRECLFFNANWEKDRTNQSGVEPCEGEQDKRLHCYASWRNSSGTIE |
| | (SEQ ID NO: 53) with 4 | LVKKGCWLDDFNCYDRQECVATEENPQVYFCCCEGNFCNERFTHLPEAGGP |
| | additional aa at N-terminus | EVTYEPPPTAPT |
| 613 | Hybrid hu-ActRIIB-ECD | GRGEAETRECLFFNANWEKDRTNQSGVEPCEGEQDKRLHCYASWRNSSGTI |
| | (SEQ ID NO: 53) with 5 | ELVKKGCWLDDFNCYDRQECVATEENPQVYFCCCEGNFCNERFTHLPEAGG |
| | additional aa at N-terminus | PEVTYEPPPTAPT |
| 614 | Hybrid hu-ActRIIB-ECD | SGRGEAETRECLFFNANWEKDRTNQSGVEPCEGEQDKRLHCYASWRNSSGT |
| | (SEQ ID NO: 53) with 6 | IELVKKGCWLDDFNCYDRQECVATEENPQVYFCCCEGNFCNERFTHLPEAG |
| | additional aa at N-terminus | GPEVTYEPPPTAPT |
| 615 | Hybrid hu-ActRIIB-ECD | AETQECLFFNANWEKDRTNQSGVEPCYGEQDKRLHCYASWRNSSGTIELVK |
| | (SEQ ID NO: 54) with 1 | KGCWLDDFNCYDRQECVATEENPQVYFCCCEGNFCNERFTHLPEAGGPEVT |
| | additional aa at N-terminus | YEPPPTAPT |
| 616 | Hybrid hu-ActRIIB-ECD | EAETQECLFFNANWEKDRTNQSGVEPCYGEQDKRLHCYASWRNSSGTIELV |
| | (SEQ ID NO: 54) with 2 | KKGCWLDDFNCYDRQECVATEENPQVYFCCCEGNFCNERFTHLPEAGGPEV |
| | additional aa at N-terminus | TYEPPPTAPT |
| 617 | Hybrid hu-ActRIIB-ECD | GEAETQECLFFNANWEKDRTNQSGVEPCYGEQDKRLHCYASWRNSSGTIEL |
| | (SEQ ID NO: 54) with 3 | VKKGCWLDDFNCYDRQECVATEENPQVYFCCCEGNFCNERFTHLPEAGGPE |
| | additional aa at N-terminus | VTYEPPPTAPT |
| 618 | Hybrid hu-ActRIIB-ECD | RGEAETQECLFFNANWEKDRINQSGVEPCYGEQDKRLHCYASWRNSSGTIE |
| | (SEQ ID NO: 54) with 4 | LVKKGCWLDDFNCYDRQECVATEENPQVYFCCCEGNFCNERFTHLPEAGGP |
| | additional aa at N-terminus | EVTYEPPPTAPT |
| 619 | Hybrid hu-ActRIIB-ECD | GRGEAETQECLFFNANWEKDRTNQSGVEPCYGEQDKRLHCYASWRNSSGTI |
| | (SEQ ID NO: 54) with 5 | ELVKKGCWLDDFNCYDRQECVATEENPQVYFCCCEGNFCNERFTHLPEAGG |
| | additional aa at N-terminus | PEVTYEPPPTAPT |
| 620 | Hybrid hu-ActRIIB-ECD | SGRGEAETQECLFFNANWEKDRTNQSGVEPCYGEQDKRLHCYASWRNSSG |
| | (SEQ ID NO: 54) with 6 | TIELVKKGCWLDDFNCYDRQECVATEENPQVYFCCCEGNFCNERFTHLPEA |
| | additional aa at N-terminus | GGPEVTYEPPPTAPT |
| 621 | Hybrid hu-ActRIIB-ECD | AETRECLFFNANWEKDRTNQSGVEPCYGDKDKRRHCYASWRNSSGTIELVK |
| | (SEQ ID NO: 55) with 1 | KGCWLDDFNCYDRQECVATEENPQVYFCCCEGNFCNERFTHLPEAGGPEVT |
| | additional aa at N-terminus | YEPPPTAPT |
| 622 | Hybrid hu-ActRIIB-ECD | EAETRECLFFNANWEKDRTNQSGVEPCYGDKDKRRHCYASWRNSSGTIELV |
| | (SEQ ID NO: 55) with 2 | KKGCWLDDFNCYDRQECVATEENPQVYFCCCEGNFCNERFTHLPEAGGPEV |
| | additional aa at N-terminus | TYEPPPTAPT |
| 623 | Hybrid hu-ActRIIB-ECD | GEAETRECLFFNANWEKDRTNQSGVEPCYGDKDKRRHCYASWRNSSGTIEL |
| | (SEQ ID NO: 55) with 3 | VKKGCWLDDFNCYDRQECVATEENPQVYFCCCEGNFCNERFTHLPEAGGPE |
| | additional aa at N-terminus | VTYEPPPTAPT |
| 624 | Hybrid hu-ActRIIB-ECD | RGEAETRECLFFNANWEKDRTNQSGVEPCYGDKDKRRHCYASWRNSSGTIE |
| | (SEQ ID NO: 55) with 4 | LVKKGCWLDDFNCYDRQECVATEENPQVYFCCCEGNFCNERFTHLPEAGGP |
| | additional aa at N-terminus | EVTYEPPPTAPT |
| 625 | Hybrid hu-ActRIIB-ECD | GRGEAETRECLFFNANWEKDRTNQSGVEPCYGDKDKRRHCYASWRNSSGTI |
| | (SEQ ID NO: 55) with 5 | ELVKKGCWLDDFNCYDRQECVATEENPQVYFCCCEGNFCNERFTHLPEAGG |
| | additional aa at N-terminus | PEVTYEPPPTAPT |
| 626 | Hybrid hu-ActRIIB-ECD | SGRGEAETRECLFFNANWEKDRTNQSGVEPCYGDKDKRRHCYASWRNSSG |
| | (SEQ ID NO: 55) with 6 | TIELVKKGCWLDDFNCYDRQECVATEENPQVYFCCCEGNFCNERFTHLPEA |
| | additional aa at N-terminus | GGPEVTYEPPPTAPT |
| 627 | Hybrid hu-ActRIIB-ECD | AETRECLFFNANWEKDRTNQTGVEPCYGDKDKRRHCFATWKNISGSIEIVK |
| | (SEQ ID NO: 56) with 1 | QGCWLDDFNCYDRQECVATEENPQVYFCCCEGNFCNERFTHLPEAGGPEVT |
| | additional aa at N-terminus | YEPPPTAPT |
| 628 | Hybrid hu-ActRIIB-ECD | EAETRECLFFNANWEKDRTNQTGVEPCYGDKDKRRHCFATWKNISGSIEIVK |
| | (SEQ ID NO: 56) with 2 | QGCWLDDFNCYDRQECVATEENPQVYFCCCEGNFCNERFTHLPEAGGPEVT |
| | additional aa at N-terminus | YEPPPTAPT |
| 629 | Hybrid hu-ActRIIB-ECD | GEAETRECLFFNANWEKDRTNQTGVEPCYGDKDKRRHCFATWKNISGSIEIV |
| | (SEQ ID NO: 56) with 3 | KQGCWLDDFNCYDRQECVATEENPQVYFCCCEGNFCNERFTHLPEAGGPEV |
| | additional aa at N-terminus | TYEPPPTAPT |
| 630 | Hybrid hu-ActRIIB-ECD | RGEAETRECLFFNANWEKDRTNQTGVEPCYGDKDKRRHCFATWKNISGSIEI |
| | (SEQ ID NO: 56) with 4 | VKQGCWLDDFNCYDRQECVATEENPQVYFCCCEGNFCNERFTHLPEAGGPE |
| | additional aa at N-terminus | VTYEPPPTAPT |
| 631 | Hybrid hu-ActRIIB-ECD | GRGEAETRECLFFNANWEKDRTNQTGVEPCYGDKDKRRHCFATWKNISGSI |
| | (SEQ ID NO: 56) with 5 | EIVKQGCWLDDFNCYDRQECVATEENPQVYFCCCEGNFCNERFTHLPEAGG |
| | additional aa at N-terminus | PEVTYEPPPTAPT |
| 632 | Hybrid hu-ActRIIB-ECD | SGRGEAETRECLFFNANWEKDRTNQTGVEPCYGDKDKRRHCFATWKNISGS |
| | (SEQ ID NO: 56) with 6 | IEIVKQGCWLDDFNCYDRQECVATEENPQVYFCCCEGNFCNERFTHLPEAG |
| | additional aa at N-terminus | GPEVTYEPPPTAPT |
| 633 | Hybrid hu-ActRIIB-ECD | AETRECLFFNANWEKDRTNQTGVEPCYGDKDKRRHCFATWKNISGSIEIVK |
| | (SEQ ID NO: 57) with 1 | QGCWLDDINCYDRQECVATEENPQVYFCCCEGNFCNERFTHLPEAGGPEVT |
| | additional aa at N-terminus | YEPPPTAPT |
| 634 | Hybrid hu-ActRIIB-ECD | EAETRECLFFNANWEKDRTNQTGVEPCYGDKDKRRHCFATWKNISGSIEIVK |
| | (SEQ ID NO: 57) with 2 | QGCWLDDINCYDRQECVATEENPQVYFCCCEGNFCNERFTHLPEAGGPEVT |
| | additional aa at N-terminus | YEPPPTAPT |
| 635 | Hybrid hu-ActRIIB-ECD | GEAETRECLFFNANWEKDRTNQTGVEPCYGDKDKRRHCFATWKNISGSIEIV |
| | (SEQ ID NO: 57) with 3 | KQGCWLDDINCYDRQECVATEENPQVYFCCCEGNFCNERFTHLPEAGGPEV |
| | additional aa at N-terminus | TYEPPPTAPT |
| 636 | Hybrid hu-ActRIIB-ECD | RGEAETRECLFFNANWEKDRTNQTGVEPCYGDKDKRRHCFATWKNISGSIEI |
| | (SEQ ID NO: 57) with 4 | VKQGCWLDDINCYDRQECVATEENPQVYFCCCEGNFCNERFTHLPEAGGPE |
| | additional aa at N-terminus | VTYEPPPTAPT |
| 637 | Hybrid hu-ActRIIB-ECD | GRGEAETRECLFFNANWEKDRTNQTGVEPCYGDKDKRRHCFATWKNISGSI |
| | (SEQ ID NO: 57) with 5 | EIVKQGCWLDDINCYDRQECVATEENPQVYFCCCEGNFCNERFTHLPEAGG |
| | additional aa at N-terminus | PEVTYEPPPTAPT |
| 638 | Hybrid hu-ActRIIB-ECD | SGRGEAETRECLFFNANWEKDRTNQTGVEPCYGDKDKRRHCFATWKNISGS |
| | (SEQ ID NO: 57) with 6 | IEIVKQGCWLDDINCYDRQECVATEENPQVYFCCCEGNFCNERFTHLPEAGG |
| | additional aa at N-terminus | PEVTYEPPPTAPT |
| 639 | Hybrid hu-ActRIIB-ECD | AETRECLFFNANWEKDRTNQTGVEPCYGDKDKRRHCFATWKNISGSIEIVK |
| | (SEQ ID NO: 58) with 1 | QGCWLDDINCYDRTDCVEKKDSPEVYFCCCEGNFCNERFTHLPEAGGPEVT |
| | additional aa at N-terminus | YEPPPTAPT |
| 640 | Hybrid hu-ActRIIB-ECD | EAETRECLFFNANWEKDRTNQTGVEPCYGDKDKRRHCFATWKNISGSIEIVK |
| | (SEQ ID NO: 58) with 2 | QGCWLDDINCYDRTDCVEKKDSPEVYFCCCEGNFCNERFTHLPEAGGPEVT |
| | additional aa at N-terminus | YEPPPTAPT |
| 641 | Hybrid hu-ActRIIB-ECD | GEAETRECLFFNANWEKDRTNQTGVEPCYGDKDKRRHCFATWKNISGSIEIV |
| | (SEQ ID NO: 58) with 3 | KQGCWLDDINCYDRTDCVEKKDSPEVYFCCCEGNFCNERFTHLPEAGGPEV |
| | additional aa at N-terminus | TYEPPPTAPT |
| 642 | Hybrid hu-ActRIIB-ECD | RGEAETRECLFFNANWEKDRTNQTGVEPCYGDKDKRRHCFATWKNISGSIEI |
| | (SEQ ID NO: 58) with 4 | VKQGCWLDDINCYDRTDCVEKKDSPEVYFCCCEGNFCNERFTHLPEAGGPE |
| | additional aa at N-terminus | VTYEPPPTAPT |
| 643 | Hybrid hu-ActRIIB-ECD | GRGEAETRECLFFNANWEKDRTNQTGVEPCYGDKDKRRHCFATWKNISGSI |
| | (SEQ ID NO: 58) with 5 | EIVKQGCWLDDINCYDRTDCVEKKDSPEVYFCCCEGNFCNERFTHLPEAGGP |
| | additional aa at N-terminus | EVTYEPPPTAPT |
| 644 | Hybrid hu-ActRIIB-ECD | SGRGEAETRECLFFNANWEKDRTNQTGVEPCYGDKDKRRHCFATWKNISGS |
| | (SEQ ID NO: 58) with 6 | IEIVKQGCWLDDINCYDRTDCVEKKDSPEVYFCCCEGNFCNERFTHLPEAGG |
| | additional aa at N-terminus | PEVTYEPPPTAPT |
| 645 | Hybrid hu-ActRIIB-ECD | AETQECIYYNANWELERTNQSGLERCYGDKDKRRHCFATWKNISGSIEIVKQ |
| | (SEQ ID NO: 59) with 1 | GCWLDDINCYDRQECVATEENPQVYFCCCEGNFCNERFTHLPEAGGPEVTY |
| | additional aa at N-terminus | EPPPTAPT |
| 646 | Hybrid hu-ActRIIB-ECD | EAETQECIYYNANWELERTNQSGLERCYGDKDKRRHCFATWKNISGSIEIVK |
| | (SEQ ID NO: 59) with 2 | QGCWLDDINCYDRQECVATEENPQVYFCCCEGNFCNERFTHLPEAGGPEVT |
| | additional aa at N-terminus | YEPPPTAPT |
| 647 | Hybrid hu-ActRIIB-ECD | GEAETQECIYYNANWELERTNQSGLERCYGDKDKRRHCFATWKNISGSIEIV |
| | (SEQ ID NO: 59) with 3 | KQGCWLDDINCYDRQECVATEENPQVYFCCCEGNFCNERFTHLPEAGGPEV |
| | additional aa at N-terminus | TYEPPPTAPT |
| 648 | Hybrid hu-ActRIIB-ECD | RGEAETQECIYYNANWELERTNQSGLERCYGDKDKRRHCFATWKNISGSIEI |
| | (SEQ ID NO: 59) with 4 | VKQGCWLDDINCYDRQECVATEENPQVYFCCCEGNFCNERFTHLPEAGGPE |
| | additional aa at N-terminus | VTYEPPPTAPT |
| 649 | Hybrid hu-ActRIIB-ECD | GRGEAETQECIYYNANWELERTNQSGLERCYGDKDKRRHCFATWKNISGSI |
| | (SEQ ID NO: 59) with 5 | EIVKQGCWLDDINCYDRQECVATEENPQVYFCCCEGNFCNERFTHLPEAGG |
| | additional aa at N-terminus | PEVTYEPPPTAPT |
| 650 | Hybrid hu-ActRIIB-ECD | SGRGEAETQECIYYNANWELERTNQSGLERCYGDKDKRRHCFATWKNISGS |
| | (SEQ ID NO: 59) with 6 | IEIVKQGCWLDDINCYDRQECVATEENPQVYFCCCEGNFCNERFTHLPEAGG |
| | additional aa at N-terminus | PEVTYEPPPTAPT |
| 651 | Hybrid hu-ActRIIB-ECD | AETRECIYYNANWELERTNQSGLERCYGDKDKRRHCFATWKNISGSIEIVKQ |
| | (SEQ ID NO: 60) with 1 | GCWLDDINCYDRTDCVEKKDSPEVYFCCCEGNFCNERFTHLPEAGGPEVTY |
| | additional aa at N-terminus | EPPPTAPT |
| 652 | Hybrid hu-ActRIIB-ECD | EAETRECIYYNANWELERTNQSGLERCYGDKDKRRHCFATWKNISGSIEIVK |
| | (SEQ ID NO: 60) with 2 | QGCWLDDINCYDRTDCVEKKDSPEVYFCCCEGNFCNERFTHLPEAGGPEVT |
| | additional aa at N-terminus | YEPPPTAPT |
| 653 | Hybrid hu-ActRIIB-ECD | GEAETRECIYYNANWELERTNQSGLERCYGDKDKRRHCFATWKNISGSIEIV |
| | (SEQ ID NO: 60) with 3 | KQGCWLDDINCYDRTDCVEKKDSPEVYFCCCEGNFCNERFTHLPEAGGPEV |
| | additional aa at N-terminus | TYEPPPTAPT |
| 654 | Hybrid hu-ActRIIB-ECD | RGEAETRECIYYNANWELERTNQSGLERCYGDKDKRRHCFATWKNISGSIEI |
| | (SEQ ID NO: 60) with 4 | VKQGCWLDDINCYDRTDCVEKKDSPEVYFCCCEGNFCNERFTHLPEAGGPE |
| | additional aa at N-terminus | VTYEPPPTAPT |
| 655 | Hybrid hu-ActRIIB-ECD | GRGEAETRECIYYNANWELERTNQSGLERCYGDKDKRRHCFATWKNISGSI |
| | (SEQ ID NO: 60) with 5 | EIVKQGCWLDDINCYDRTDCVEKKDSPEVYFCCCEGNFCNERFTHLPEAGGP |
| | additional aa at N-terminus | EVTYEPPPTAPT |
| 656 | Hybrid hu-ActRIIB-ECD | SGRGEAETRECIYYNANWELERTNQSGLERCYGDKDKRRHCFATWKNISGS |
| | (SEQ ID NO: 60) with 6 | IEIVKQGCWLDDINCYDRTDCVEKKDSPEVYFCCCEGNFCNERFTHLPEAGG |
| | additional aa at N-terminus | PEVTYEPPPTAPT |
| 657 | Hybrid hu-ActRIIB-ECD | AETRECIYYNANWELERTNQSGLERCYGDKDKRRHCFATWKNISGSIEIVKQ |
| | (SEQ ID NO: 61) with 1 | GCWLDDINCYDRTDCVEKKDSPEVYFCCCEGNMCNERFTHLPEAGGPEVTY |
| | additional aa at N-terminus | EPPPTAPT |
| 658 | Hybrid hu-ActRIIB-ECD | EAETRECIYYNANWELERTNQSGLERCYGDKDKRRHCFATWKNISGSIEIVK |
| | (SEQ ID NO: 61) with 2 | QGCWLDDINCYDRTDCVEKKDSPEVYFCCCEGNMCNERFTHLPEAGGPEVT |
| | additional aa at N-terminus | YEPPPTAPT |
| 659 | Hybrid hu-ActRIIB-ECD | GEAETRECIYYNANWELERTNQSGLERCYGDKDKRRHCFATWKNISGSIEIV |
| | (SEQ ID NO: 61) with 3 | KQGCWLDDINCYDRTDCVEKKDSPEVYFCCCEGNMCNERFTHLPEAGGPEV |
| | additional aa at N-terminus | TYEPPPTAPT |
| 660 | Hybrid hu-ActRIIB-ECD | RGEAETRECIYYNANWELERTNQSGLERCYGDKDKRRHCFATWKNISGSIEI |
| | (SEQ ID NO: 61) with 4 | VKQGCWLDDINCYDRTDCVEKKDSPEVYFCCCEGNMCNERFTHLPEAGGPE |
| | additional aa at N-terminus | VTYEPPPTAPT |
| 661 | Hybrid hu-ActRIIB-ECD | GRGEAETRECIYYNANWELERTNQSGLERCYGDKDKRRHCFATWKNISGSI |
| | (SEQ ID NO: 61) with 5 | EIVKQGCWLDDINCYDRTDCVEKKDSPEVYFCCCEGNMCNERFTHLPEAGG |
| | additional aa at N-terminus | PEVTYEPPPTAPT |
| 662 | Hybrid hu-ActRIIB-ECD | SGRGEAETRECIYYNANWELERTNQSGLERCYGDKDKRRHCFATWKNISGS |
| | (SEQ ID NO: 61) with 6 | IEIVKQGCWLDDINCYDRTDCVEKKDSPEVYFCCCEGNMCNERFTHLPEAG |
| | additional aa at N-terminus | GPEVTYEPPPTAPT |
| 663 | Hybrid hu-ActRIIB-ECD | AETRECIYYNANWELERTNQSGLERCEGEQDKRLHCFATWKNISGSIEIVKQ |
| | (SEQ ID NO: 62) with 1 | GCWLDDINCYDRTDCVEKKDSPEVYFCCCEGNFCNERFTHLPEAGGPEVTY |
| | additional aa at N-terminus | EPPPTAPT |
| 664 | Hybrid hu-ActRIIB-ECD | EAETRECIYYNANWELERTNQSGLERCEGEQDKRLHCFATWKNISGSIEIVK |
| | (SEQ ID NO: 62) with 2 | QGCWLDDINCYDRTDCVEKKDSPEVYFCCCEGNFCNERFTHLPEAGGPEVT |
| | additional aa at N-terminus | YEPPPTAPT |
| 665 | Hybrid hu-ActRIIB-ECD | GEAETRECIYYNANWELERTNQSGLERCEGEQDKRLHCFATWKNISGSIEIV |
| | (SEQ ID NO: 62) with 3 | KQGCWLDDINCYDRTDCVEKKDSPEVYFCCCEGNFCNERFTHLPEAGGPEV |
| | additional aa at N-terminus | TYEPPPTAPT |
| 666 | Hybrid hu-ActRIIB-ECD | RGEAETRECIYYNANWELERTNQSGLERCEGEQDKRLHCFATWKNISGSIEI |
| | (SEQ ID NO: 62) with 4 | VKQGCWLDDINCYDRTDCVEKKDSPEVYFCCCEGNFCNERFTHLPEAGGPE |
| | additional aa at N-terminus | VTYEPPPTAPT |
| 667 | Hybrid hu-ActRIIB-ECD | GRGEAETRECIYYNANWELERTNQSGLERCEGEQDKRLHCFATWKNISGSIE |
| | (SEQ ID NO: 62) with 5 | IVKQGCWLDDINCYDRTDCVEKKDSPEVYFCCCEGNFCNERFTHLPEAGGPE |
| | additional aa at N-terminus | VTYEPPPTAPT |
| 668 | Hybrid hu-ActRIIB-ECD | SGRGEAETRECIYYNANWELERTNQSGLERCEGEQDKRLHCFATWKNISGSI |
| | (SEQ ID NO: 62) with 6 | EIVKQGCWLDDINCYDRTDCVEKKDSPEVYFCCCEGNFCNERFTHLPEAGGP |
| | additional aa at N-terminus | EVTYEPPPTAPT |
| 669 | Hybrid hu-ActRIIB-ECD | AETRECIYYNANWELERTNQSGLERCEGEQDKRLHCFATWKNISGSIEIVKQ |
| | (SEQ ID NO: 63) with 1 | GCWLDDINCYDRTDCVATEENPQVYFCCCEGNFCNERFTHLPEAGGPEVTY |
| | additional aa at N-terminus | EPPPTAPT |
| 670 | Hybrid hu-ActRIIB-ECD | EAETRECIYYNANWELERTNQSGLERCEGEQDKRLHCFATWKNISGSIEIVK |
| | (SEQ ID NO: 63) with 2 | QGCWLDDINCYDRTDCVATEENPQVYFCCCEGNFCNERFTHLPEAGGPEVT |
| | additional aa at N-terminus | YEPPPTAPT |
| 671 | Hybrid hu-ActRIIB-ECD | GEAETRECIYYNANWELERTNQSGLERCEGEQDKRLHCFATWKNISGSIEIV |
| | (SEQ ID NO: 63) with 3 | KQGCWLDDINCYDRTDCVATEENPQVYFCCCEGNFCNERFTHLPEAGGPEV |
| | additional aa at N-terminus | TYEPPPTAPT |
| 672 | Hybrid hu-ActRIIB-ECD | RGEAETRECIYYNANWELERTNQSGLERCEGEQDKRLHCFATWKNISGSIEI |
| | (SEQ ID NO: 63) with 4 | VKQGCWLDDINCYDRTDCVATEENPQVYFCCCEGNFCNERFTHLPEAGGPE |
| | additional aa at N-terminus | VTYEPPPTAPT |
| 673 | Hybrid hu-ActRIIB-ECD | GRGEAETRECIYYNANWELERTNQSGLERCEGEQDKRLHCFATWKNISGSIE |
| | (SEQ ID NO: 63) with 5 | IVKQGCWLDDINCYDRTDCVATEENPQVYFCCCEGNFCNERFTHLPEAGGP |
| | additional aa at N-terminus | EVTYEPPPTAPT |
| 674 | Hybrid hu-ActRIIB-ECD | SGRGEAETRECIYYNANWELERTNQSGLERCEGEQDKRLHCFATWKNISGSI |
| | (SEQ ID NO: 63) with 6 | EIVKQGCWLDDINCYDRTDCVATEENPQVYFCCCEGNFCNERFTHLPEAGG |
| | additional aa at N-terminus | PEVTYEPPPTAPT |
| 675 | Hybrid hu-ActRIIB-ECD | AETRECIYYNANWELERTNQSGLERCEGEQDKRLHCFATWKNISGSIEIVKQ |
| | (SEQ ID NO: 64) with 1 | GCWLDDFNCYDRTDCVATEENPQVYFCCCEGNFCNERFTHLPEAGGPEVTY |
| | additional aa at N-terminus | EPPPTAPT |
| 676 | Hybrid hu-ActRIIB-ECD | EAETRECIYYNANWELERTNQSGLERCEGEQDKRLHCFATWKNISGSIEIVK |
| | (SEQ ID NO: 64) with 2 | QGCWLDDFNCYDRTDCVATEENPQVYFCCCEGNFCNERFTHLPEAGGPEVT |
| | additional aa at N-terminus | YEPPPTAPT |
| 677 | Hybrid hu-ActRIIB-ECD | GEAETRECIYYNANWELERTNQSGLERCEGEQDKRLHCFATWKNISGSIEIV |
| | (SEQ ID NO: 64) with 3 | KQGCWLDDFNCYDRTDCVATEENPQVYFCCCEGNFCNERFTHLPEAGGPEV |
| | additional aa at N-terminus | TYEPPPTAPT |
| 678 | Hybrid hu-ActRIIB-ECD | RGEAETRECIYYNANWELERTNQSGLERCEGEQDKRLHCFATWKNISGSIEI |
| | (SEQ ID NO: 64) with 4 | VKQGCWLDDFNCYDRTDCVATEENPQVYFCCCEGNFCNERFTHLPEAGGPE |
| | additional aa at N-terminus | VTYEPPPTAPT |
| 679 | Hybrid hu-ActRIIB-ECD | GRGEAETRECIYYNANWELERTNQSGLERCEGEQDKRLHCFATWKNISGSIE |
| | (SEQ ID NO: 64) with 5 | IVKQGCWLDDFNCYDRTDCVATEENPQVYFCCCEGNFCNERFTHLPEAGGP |
| | additional aa at N-terminus | EVTYEPPPTAPT |
| 680 | Hybrid hu-ActRIIB-ECD | SGRGEAETRECIYYNANWELERTNQSGLERCEGEQDKRLHCFATWKNISGSI |
| | (SEQ ID NO: 64) with 6 | EIVKQGCWLDDFNCYDRTDCVATEENPQVYFCCCEGNFCNERFTHLPEAGG |
| | additional aa at N-terminus | PEVTYEPPPTAPT |
| 681 | Hybrid hu-ActRIIB-ECD | AETRECIYYNANWELERTNQSGLERCEGEQDKRLHCFATWKNISGSIEIVKQ |
| | (SEQ ID NO: 65) with 1 | GCWLDDFNCYDRQECVATEENPQVYFCCCEGNFCNERFTHLPEAGGPEVTY |
| | additional aa at N-terminus | EPPPTAPT |
| 682 | Hybrid hu-ActRIIB-ECD | EAETRECIYYNANWELERTNQSGLERCEGEQDKRLHCFATWKNISGSIEIVK |
| | (SEQ ID NO: 65) with 2 | QGCWLDDFNCYDRQECVATEENPQVYFCCCEGNFCNERFTHLPEAGGPEVT |
| | additional aa at N-terminus | YEPPPTAPT |
| 683 | Hybrid hu-ActRIIB-ECD | GEAETRECIYYNANWELERTNQSGLERCEGEQDKRLHCFATWKNISGSIEIV |
| | (SEQ ID NO: 65) with 3 | KQGCWLDDFNCYDRQECVATEENPQVYFCCCEGNFCNERFTHLPEAGGPEV |
| | additional aa at N-terminus | TYEPPPTAPT |
| 684 | Hybrid hu-ActRIIB-ECD | RGEAETRECIYYNANWELERTNQSGLERCEGEQDKRLHCFATWKNISGSIEI |
| | (SEQ ID NO: 65) with 4 | VKQGCWLDDFNCYDRQECVATEENPQVYFCCCEGNFCNERFTHLPEAGGPE |
| | additional aa at N-terminus | VTYEPPPTAPT |
| 685 | Hybrid hu-ActRIIB-ECD | GRGEAETRECIYYNANWELERTNQSGLERCEGEQDKRLHCFATWKNISGSIE |
| | (SEQ ID NO: 65) with 5 | IVKQGCWLDDFNCYDRQECVATEENPQVYFCCCEGNFCNERFTHLPEAGGP |
| | additional aa at N-terminus | EVTYEPPPTAPT |
| 686 | Hybrid hu-ActRIIB-ECD | SGRGEAETRECIYYNANWELERTNQSGLERCEGEQDKRLHCFATWKNISGSI |
| | (SEQ ID NO: 65) with 6 | EIVKQGCWLDDFNCYDRQECVATEENPQVYFCCCEGNFCNERFTHLPEAGG |
| | additional aa at N-terminus | PEVTYEPPPTAPT |
| 687 | Hybrid hu-ActRIIB-ECD | AETQECIYYNANWELERTNQSGLERCYGDKDKRRHCYASWRNSSGTIELVK |
| | (SEQ ID NO: 66) with 1 | KGCWLDDINCYDRTDCVEKKDSPEVYFCCCEGNFCNERFTHLPEAGGPEVT |
| | additional aa at N-terminus | YEPPPTAPT |
| 688 | Hybrid hu-ActRIIB-ECD | EAETQECIYYNANWELERTNQSGLERCYGDKDKRRHCYASWRNSSGTIELV |
| | (SEQ ID NO: 66) with 2 | KKGCWLDDINCYDRTDCVEKKDSPEVYFCCCEGNFCNERFTHLPEAGGPEV |
| | additional aa at N-terminus | TYEPPPTAPT |
| 689 | Hybrid hu-ActRIIB-ECD | GEAETQECIYYNANWELERTNQSGLERCYGDKDKRRHCYASWRNSSGTIEL |
| | (SEQ ID NO: 66) with 3 | VKKGCWLDDINCYDRTDCVEKKDSPEVYFCCCEGNFCNERFTHLPEAGGPE |
| | additional aa at N-terminus | VTYEPPPTAPT |
| 690 | Hybrid hu-ActRIIB-ECD | RGEAETQECIYYNANWELERTNQSGLERCYGDKDKRRHCYASWRNSSGTIE |
| | (SEQ ID NO: 66) with 4 | LVKKGCWLDDINCYDRTDCVEKKDSPEVYFCCCEGNFCNERFTHLPEAGGP |
| | additional aa at N-terminus | EVTYEPPPTAPT |
| 691 | Hybrid hu-ActRIIB-ECD | GRGEAETQECIYYNANWELERTNQSGLERCYGDKDKRRHCYASWRNSSGTI |
| | (SEQ ID NO: 66) with 5 | ELVKKGCWLDDINCYDRTDCVEKKDSPEVYFCCCEGNFCNERFTHLPEAGG |
| | additional aa at N-terminus | PEVTYEPPPTAPT |
| 692 | Hybrid hu-ActRIIB-ECD | SGRGEAETQECIYYNANWELERTNQSGLERCYGDKDKRRHCYASWRNSSG |
| | (SEQ ID NO: 66) with 6 | TIELVKKGCWLDDINCYDRTDCVEKKDSPEVYFCCCEGNFCNERFTHLPEAG |
| | additional aa at N-terminus | GPEVTYEPPPTAPT |
| 693 | Hybrid hu-ActRIIB-ECD | AETQECIYYNANWELERTNQSGLERCYGDKDKRRHCYASWRNSSGTIELVK |
| | (SEQ ID NO: 67) with 1 | KGCWLDDINCYDRTDCVEKKDSPEVYFCCCEGNMCNERFTHLPEAGGPEVT |
| | additional aa at N-terminus | YEPPPTAPT |
| 694 | Hybrid hu-ActRIIB-ECD | EAETQECIYYNANWELERTNQSGLERCYGDKDKRRHCYASWRNSSGTIELV |
| | (SEQ ID NO: 67) with 2 | KKGCWLDDINCYDRTDCVEKKDSPEVYFCCCEGNMCNERFTHLPEAGGPEV |
| | additional aa at N-terminus | TYEPPPTAPT |
| 695 | Hybrid hu-ActRIIB-ECD | GEAETQECIYYNANWELERTNQSGLERCYGDKDKRRHCYASWRNSSGTIEL |
| | (SEQ ID NO: 67) with 3 | VKKGCWLDDINCYDRTDCVEKKDSPEVYFCCCEGNMCNERFTHLPEAGGPE |
| | additional aa at N-terminus | VTYEPPPTAPT |
| 696 | Hybrid hu-ActRIIB-ECD | RGEAETQECIYYNANWELERTNQSGLERCYGDKDKRRHCYASWRNSSGTIE |
| | (SEQ ID NO: 67) with 4 | LVKKGCWLDDINCYDRTDCVEKKDSPEVYFCCCEGNMCNERFTHLPEAGGP |
| | additional aa at N-terminus | EVTYEPPPTAPT |
| 697 | Hybrid hu-ActRIIB-ECD | GRGEAETQECIYYNANWELERTNQSGLERCYGDKDKRRHCYASWRNSSGTI |
| | (SEQ ID NO: 67) with 5 | ELVKKGCWLDDINCYDRTDCVEKKDSPEVYFCCCEGNMCNERFTHLPEAG |
| | additional aa at N-terminus | GPEVTYEPPPTAPT |
| 698 | Hybrid hu-ActRIIB-ECD | SGRGEAETQECIYYNANWELERTNQSGLERCYGDKDKRRHCYASWRNSSG |
| | (SEQ ID NO: 67) with 6 | TIELVKKGCWLDDINCYDRTDCVEKKDSPEVYFCCCEGNMCNERFTHLPEA |
| | additional aa at N-terminus | GGPEVTYEPPPTAPT |
| 699 | Hybrid hu-ActRIIB-ECD | AETQECIYYNANWELERTNQSGLERCYGDKDKRRHCFATWKNISGSIEIVKQ |
| | (SEQ ID NO: 68) with 1 | GCWLDDINCYDRTDCVEKKDSPEVYFCCCEGNMCNERFTHLPEAGGPEVTY |
| | additional aa at N-terminus | EPPPTAPT |
| 700 | Hybrid hu-ActRIIB-ECD | EAETQECIYYNANWELERTNQSGLERCYGDKDKRRHCFATWKNISGSIEIVK |
| | (SEQ ID NO: 68) with 2 | QGCWLDDINCYDRTDCVEKKDSPEVYFCCCEGNMCNERFTHLPEAGGPEVT |
| | additional aa at N-terminus | YEPPPTAPT |
| 701 | Hybrid hu-ActRIIB-ECD | GEAETQECIYYNANWELERTNQSGLERCYGDKDKRRHCFATWKNISGSIEIV |
| | (SEQ ID NO: 68) with 3 | KQGCWLDDINCYDRTDCVEKKDSPEVYFCCCEGNMCNERFTHLPEAGGPEV |
| | additional aa at N-terminus | TYEPPPTAPT |
| 702 | Hybrid hu-ActRIIB-ECD | RGEAETQECIYYNANWELERTNQSGLERCYGDKDKRRHCFATWKNISGSIEI |
| | (SEQ ID NO: 68) with 4 | VKQGCWLDDINCYDRTDCVEKKDSPEVYFCCCEGNMCNERFTHLPEAGGPE |
| | additional aa at N-terminus | VTYEPPPTAPT |
| 703 | Hybrid hu-ActRIIB-ECD | GRGEAETQECIYYNANWELERTNQSGLERCYGDKDKRRHCFATWKNISGSI |
| | (SEQ ID NO: 68) with 5 | EIVKQGCWLDDINCYDRTDCVEKKDSPEVYFCCCEGNMCNERFTHLPEAGG |
| | additional aa at N-terminus | PEVTYEPPPTAPT |
| 704 | Hybrid hu-ActRIIB-ECD | SGRGEAETQECIYYNANWELERTNQSGLERCYGDKDKRRHCFATWKNISGS |
| | (SEQ ID NO: 68) with 6 | IEIVKQGCWLDDINCYDRTDCVEKKDSPEVYFCCCEGNMCNERFTHLPEAG |
| | additional aa at N-terminus | GPEVTYEPPPTAPT |
| 705 | Hybrid hu-ActRIIB-ECD | AETQECIYYNANWELERTNQSGLERCYGDKDKRRHCFATWKNISGSIEIVKQ |
| | (SEQ ID NO: 69) with 1 | GCWLDDINCYDRTDCVEKKDSPEVYFCCCEGNFCNERFTHLPEAGGPEVTY |
| | additional aa at N-terminus | EPPPTAPT |
| 706 | Hybrid hu-ActRIIB-ECD | EAETQECIYYNANWELERTNQSGLERCYGDKDKRRHCFATWKNISGSIEIVK |
| | (SEQ ID NO: 69) with 2 | QGCWLDDINCYDRTDCVEKKDSPEVYFCCCEGNFCNERFTHLPEAGGPEVT |
| | additional aa at N-terminus | YEPPPTAPT |
| 707 | Hybrid hu-ActRIIB-ECD | GEAETQECIYYNANWELERTNQSGLERCYGDKDKRRHCFATWKNISGSIEIV |
| | (SEQ ID NO: 69) with 3 | KQGCWLDDINCYDRTDCVEKKDSPEVYFCCCEGNFCNERFTHLPEAGGPEV |
| | additional aa at N-terminus | TYEPPPTAPT |
| 708 | Hybrid hu-ActRIIB-ECD | RGEAETQECIYYNANWELERTNQSGLERCYGDKDKRRHCFATWKNISGSIEI |
| | (SEQ ID NO: 69) with 4 | VKQGCWLDDINCYDRTDCVEKKDSPEVYFCCCEGNFCNERFTHLPEAGGPE |
| | additional aa at N-terminus | VTYEPPPTAPT |
| 709 | Hybrid hu-ActRIIB-ECD | GRGEAETQECIYYNANWELERTNQSGLERCYGDKDKRRHCFATWKNISGSI |
| | (SEQ ID NO: 69) with 5 | EIVKQGCWLDDINCYDRTDCVEKKDSPEVYFCCCEGNFCNERFTHLPEAGGP |
| | additional aa at N-terminus | EVTYEPPPTAPT |
| 710 | Hybrid hu-ActRIIB-ECD | SGRGEAETQECIYYNANWELERTNQSGLERCYGDKDKRRHCFATWKNISGS |
| | (SEQ ID NO: 69) with 6 | IEIVKQGCWLDDINCYDRTDCVEKKDSPEVYFCCCEGNFCNERFTHLPEAGG |
| | additional aa at N-terminus | PEVTYEPPPTAPT |
| 711 | Hybrid hu-ActRIIB-ECD | AETRECLFFNANWEKDRTNQTGVEPCEGEQDKRLHCFATWKNISGSIEIVKQ |
| | (SEQ ID NO: 70) with 1 | GCWLDDINCYDRTDCVEKKDSPEVYFCCCEGNFCNERFTHLPEAGGPEVTY |
| | additional aa at N-terminus | EPPPTAPT |
| 712 | Hybrid hu-ActRIIB-ECD | EAETRECLFFNANWEKDRTNQTGVEPCEGEQDKRLHCFATWKNISGSIEIVK |
| | (SEQ ID NO: 70) with 2 | QGCWLDDINCYDRTDCVEKKDSPEVYFCCCEGNFCNERFTHLPEAGGPEVT |
| | additional aa at N-terminus | YEPPPTAPT |
| 713 | Hybrid hu-ActRIIB-ECD | GEAETRECLFFNANWEKDRTNQTGVEPCEGEQDKRLHCFATWKNISGSIEIV |
| | (SEQ ID NO: 70) with 3 | KQGCWLDDINCYDRTDCVEKKDSPEVYFCCCEGNFCNERFTHLPEAGGPEV |
| | additional aa at N-terminus | TYEPPPTAPT |
| 714 | Hybrid hu-ActRIIB-ECD | RGEAETRECLFFNANWEKDRTNQTGVEPCEGEQDKRLHCFATWKNISGSIEI |
| | (SEQ ID NO: 70) with 4 | VKQGCWLDDINCYDRTDCVEKKDSPEVYFCCCEGNFCNERFTHLPEAGGPE |
| | additional aa at N-terminus | VTYEPPPTAPT |
| 715 | Hybrid hu-ActRIIB-ECD | GRGEAETRECLFFNANWEKDRTNQTGVEPCEGEQDKRLHCFATWKNISGSI |
| | (SEQ ID NO: 70) with 5 | EIVKQGCWLDDINCYDRTDCVEKKDSPEVYFCCCEGNFCNERFTHLPEAGGP |
| | additional aa at N-terminus | EVTYEPPPTAPT |
| 716 | Hybrid hu-ActRIIB-ECD | SGRGEAETRECLFFNANWEKDRTNQTGVEPCEGEQDKRLHCFATWKNISGSI |
| | (SEQ ID NO: 70) with 6 | EIVKQGCWLDDINCYDRTDCVEKKDSPEVYFCCCEGNFCNERFTHLPEAGGP |
| | additional aa at N-terminus | EVTYEPPPTAPT |
| 717 | Hybrid hu-ActRIIB-ECD | AETRECLFFNANWEKDRTNQSGVEPCEGEQDKRLHCYASWRNSSGTIELVK |
| | (SEQ ID NO: 71) with 1 | KGCWLDDINCYDRTDCVEKKDSPEVYFCCCEGNFCNERFTHLPEAGGPEVT |
| | additional aa at N-terminus | YEPPPTAPT |
| 718 | Hybrid hu-ActRIIB-ECD | EAETRECLFFNANWEKDRTNQSGVEPCEGEQDKRLHCYASWRNSSGTIELV |
| | (SEQ ID NO: 71) with 2 | KKGCWLDDINCYDRTDCVEKKDSPEVYFCCCEGNFCNERFTHLPEAGGPEV |
| | additional aa at N-terminus | TYEPPPTAPT |
| 719 | Hybrid hu-ActRIIB-ECD | GEAETRECLFFNANWEKDRTNQSGVEPCEGEQDKRLHCYASWRNSSGTIEL |
| | (SEQ ID NO: 71) with 3 | VKKGCWLDDINCYDRTDCVEKKDSPEVYFCCCEGNFCNERFTHLPEAGGPE |
| | additional aa at N-terminus | VTYEPPPTAPT |
| 720 | Hybrid hu-ActRIIB-ECD | RGEAETRECLFFNANWEKDRTNQSGVEPCEGEQDKRLHCYASWRNSSGTIE |
| | (SEQ ID NO: 71) with 4 | LVKKGCWLDDINCYDRTDCVEKKDSPEVYFCCCEGNFCNERFTHLPEAGGP |
| | additional aa at N-terminus | EVTYEPPPTAPT |
| 721 | Hybrid hu-ActRIIB-ECD | GRGEAETRECLFFNANWEKDRTNQSGVEPCEGEQDKRLHCYASWRNSSGTI |
| | (SEQ ID NO: 71) with 5 | ELVKKGCWLDDINCYDRTDCVEKKDSPEVYFCCCEGNFCNERFTHLPEAGG |
| | additional aa at N-terminus | PEVTYEPPPTAPT |
| 722 | Hybrid hu-ActRIIB-ECD | SGRGEAETRECLFFNANWEKDRTNQSGVEPCEGEQDKRLHCYASWRNSSGT |
| | (SEQ ID NO: 71) with 6 | IELVKKGCWLDDINCYDRTDCVEKKDSPEVYFCCCEGNFCNERFTHLPEAG |
| | additional aa at N-terminus | GPEVTYEPPPTAPT |
| 723 | Hybrid hu-ActRIIB-ECD | AETRECLFFNANWEKDRTNQSGVEPCYGDKDKRRHCYASWRNSSGTIELVK |
| | (SEQ ID NO: 72) with 1 | KGCWLDDINCYDRTDCVEKKDSPEVYFCCCEGNFCNERFTHLPEAGGPEVT |
| | additional aa at N-terminus | YEPPPTAPT |
| 724 | Hybrid hu-ActRIIB-ECD | EAETRECLFFNANWEKDRTNQSGVEPCYGDKDKRRHCYASWRNSSGTIELV |
| | (SEQ ID NO: 72) with 2 | KKGCWLDDINCYDRTDCVEKKDSPEVYFCCCEGNFCNERFTHLPEAGGPEV |
| | additional aa at N-terminus | TYEPPPTAPT |
| 725 | Hybrid hu-ActRIIB-ECD | GEAETRECLFFNANWEKDRTNQSGVEPCYGDKDKRRHCYASWRNSSGTIEL |
| | (SEQ ID NO: 72) with 3 | VKKGCWLDDINCYDRTDCVEKKDSPEVYFCCCEGNFCNERFTHLPEAGGPE |
| | additional aa at N-terminus | VTYEPPPTAPT |
| 726 | Hybrid hu-ActRIIB-ECD | RGEAETRECLFFNANWEKDRTNQSGVEPCYGDKDKRRHCYASWRNSSGTIE |
| | (SEQ ID NO: 72) with 4 | LVKKGCWLDDINCYDRTDCVEKKDSPEVYFCCCEGNFCNERFTHLPEAGGP |
| | additional aa at N-terminus | EVTYEPPPTAPT |
| 727 | Hybrid hu-ActRIIB-ECD | GRGEAETRECLFFNANWEKDRTNQSGVEPCYGDKDKRRHCYASWRNSSGTI |
| | (SEQ ID NO: 72) with 5 | ELVKKGCWLDDINCYDRTDCVEKKDSPEVYFCCCEGNFCNERFTHLPEAGG |
| | additional aa at N-terminus | PEVTYEPPPTAPT |
| 728 | Hybrid hu-ActRIIB-ECD | SGRGEAETRECLFFNANWEKDRTNQSGVEPCYGDKDKRRHCYASWRNSSG |
| | (SEQ ID NO: 72) with 6 | TIELVKKGCWLDDINCYDRTDCVEKKDSPEVYFCCCEGNFCNERFTHLPEAG |
| | additional aa at N-terminus | GPEVTYEPPPTAPT |
| 729 | Hybrid hu-ActRIIB-ECD | AETRECIYYNANWELERTNQSGLERCYGDKDKRRHCYASWRNSSGTIELVK |
| | (SEQ ID NO: 73) with 1 | KGCWLDDFNCYDRTDCVATEENPQVYFCCCEGNFCNERFTHLPEAGGPEVT |
| | additional aa at N-terminus | YEPPPTAPT |
| 730 | Hybrid hu-ActRIIB-ECD | EAETRECIYYNANWELERTNQSGLERCYGDKDKRRHCYASWRNSSGTIELV |
| | (SEQ ID NO: 73) with 2 | KKGCWLDDFNCYDRTDCVATEENPQVYFCCCEGNFCNERFTHLPEAGGPEV |
| | additional aa at N-terminus | TYEPPPTAPT |
| 731 | Hybrid hu-ActRIIB-ECD | GEAETRECIYYNANWELERTNQSGLERCYGDKDKRRHCYASWRNSSGTIEL |
| | (SEQ ID NO: 73) with 3 | VKKGCWLDDFNCYDRTDCVATEENPQVYFCCCEGNFCNERFTHLPEAGGPE |
| | additional aa at N-terminus | VTYEPPPTAPT |
| 732 | Hybrid hu-ActRIIB-ECD | RGEAETRECIYYNANWELERTNQSGLERCYGDKDKRRHCYASWRNSSGTIE |
| | (SEQ ID NO: 73) with 4 | LVKKGCWLDDFNCYDRTDCVATEENPQVYFCCCEGNFCNERFTHLPEAGGP |
| | additional aa at N-terminus | EVTYEPPPTAPT |
| 733 | Hybrid hu-ActRIIB-ECD | GRGEAETRECIYYNANWELERTNQSGLERCYGDKDKRRHCYASWRNSSGTI |
| | (SEQ ID NO: 73) with 5 | ELVKKGCWLDDFNCYDRTDCVATEENPQVYFCCCEGNFCNERFTHLPEAGG |
| | additional aa at N-terminus | PEVTYEPPPTAPT |
| 734 | Hybrid hu-ActRIIB-ECD | SGRGEAETRECIYYNANWELERTNQSGLERCYGDKDKRRHCYASWRNSSG |
| | (SEQ ID NO: 73) with 6 | TIELVKKGCWLDDFNCYDRTDCVATEENPQVYFCCCEGNFCNERFTHLPEA |
| | additional aa at N-terminus | GGPEVTYEPPPTAPT |
| 735 | Hybrid hu-ActRIIB-ECD | AETRECIYYNANWELERTNQSGLERCYGDKDKRRHCYASWRNSSGTIELVK |
| | (SEQ ID NO: 74) with 1 | QGCWLDDFNCYDRTDCVATEENPQVYFCCCEGNFCNERFTHLPEAGGPEVT |
| | additional aa at N-terminus | YEPPPTAPT |
| 736 | Hybrid hu-ActRIIB-ECD | EAETRECIYYNANWELERTNQSGLERCYGDKDKRRHCYASWRNSSGTIELV |
| | (SEQ ID NO: 74) with 2 | KQGCWLDDFNCYDRTDCVATEENPQVYFCCCEGNFCNERFTHLPEAGGPEV |
| | additional aa at N-terminus | TYEPPPTAPT |
| 737 | Hybrid hu-ActRIIB-ECD | GEAETRECIYYNANWELERTNQSGLERCYGDKDKRRHCYASWRNSSGTIEL |
| | (SEQ ID NO: 74) with 3 | VKQGCWLDDFNCYDRTDCVATEENPQVYFCCCEGNFCNERFTHLPEAGGPE |
| | additional aa at N-terminus | VTYEPPPTAPT |
| 738 | Hybrid hu-ActRIIB-ECD | RGEAETRECIYYNANWELERTNQSGLERCYGDKDKRRHCYASWRNSSGTIE |
| | (SEQ ID NO: 74) with 4 | LVKQGCWLDDFNCYDRTDCVATEENPQVYFCCCEGNFCNERFTHLPEAGGP |
| | additional aa at N-terminus | EVTYEPPPTAPT |
| 739 | Hybrid hu-ActRIIB-ECD | GRGEAETRECIYYNANWELERTNQSGLERCYGDKDKRRHCYASWRNSSGTI |
| | (SEQ ID NO: 74) with 5 | ELVKQGCWLDDFNCYDRTDCVATEENPQVYFCCCEGNFCNERFTHLPEAGG |
| | additional aa at N-terminus | PEVTYEPPPTAPT |
| 740 | Hybrid hu-ActRIIB-ECD | SGRGEAETRECIYYNANWELERTNQSGLERCYGDKDKRRHCYASWRNSSG |
| | (SEQ ID NO: 74) with 6 | TIELVKQGCWLDDFNCYDRTDCVATEENPQVYFCCCEGNFCNERFTHLPEA |
| | additional aa at N-terminus | GGPEVTYEPPPTAPT |
| 741 | Hybrid hu-ActRIIB-ECD | AETRECIYYNANWELERTNQSGLERCYGDKDKRRHCYASWRNSSGTIEIVK |
| | (SEQ ID NO: 75) with 1 | KGCWLDDFNCYDRTDCVATEENPQVYFCCCEGNFCNERFTHLPEAGGPEVT |
| | additional aa at N-terminus | YEPPPTAPT |
| 742 | Hybrid hu-ActRIIB-ECD | EAETRECIYYNANWELERINQSGLERCYGDKDKRRHCYASWRNSSGTIEIV |
| | (SEQ ID NO: 75) with 2 | KKGCWLDDFNCYDRTDCVATEENPQVYFCCCEGNFCNERFTHLPEAGGPEV |
| | additional aa at N-terminus | TYEPPPTAPT |
| 743 | Hybrid hu-ActRIIB-ECD | GEAETRECIYYNANWELERTNQSGLERCYGDKDKRRHCYASWRNSSGTIEI |
| | (SEQ ID NO: 75) with 3 | VKKGCWLDDFNCYDRTDCVATEENPQVYFCCCEGNFCNERFTHLPEAGGPE |
| | additional aa at N-terminus | VTYEPPPTAPT |
| 744 | Hybrid hu-ActRIIB-ECD | RGEAETRECIYYNANWELERTNQSGLERCYGDKDKRRHCYASWRNSSGTIE |
| | (SEQ ID NO: 75) with 4 | IVKKGCWLDDFNCYDRTDCVATEENPQVYFCCCEGNFCNERFTHLPEAGGP |
| | additional aa at N-terminus | EVTYEPPPTAPT |
| 745 | Hybrid hu-ActRIIB-ECD | GRGEAETRECIYYNANWELERTNQSGLERCYGDKDKRRHCYASWRNSSGTI |
| | (SEQ ID NO: 75) with 5 | EIVKKGCWLDDFNCYDRTDCVATEENPQVYFCCCEGNFCNERFTHLPEAGG |
| | additional aa at N-terminus | PEVTYEPPPTAPT |
| 746 | Hybrid hu-ActRIIB-ECD | SGRGEAETRECIYYNANWELERTNQSGLERCYGDKDKRRHCYASWRNSSG |
| | (SEQ ID NO: 75) with 6 | TIEIVKKGCWLDDFNCYDRTDCVATEENPQVYFCCCEGNFCNERFTHLPEAG |
| | additional aa at N-terminus | GPEVTYEPPPTAPT |
| 747 | Hybrid hu-ActRIIB-ECD | AETRECIYYNANWELERTNQSGLERCYGDKDKRRHCYASWRNSSGSIELVK |
| | (SEQ ID NO: 76) with 1 | KGCWLDDFNCYDRTDCVATEENPQVYFCCCEGNFCNERFTHLPEAGGPEVT |
| | additional aa at N-terminus | YEPPPTAPT |
| 748 | Hybrid hu-ActRIIB-ECD | EAETRECIYYNANWELERTNQSGLERCYGDKDKRRHCYASWRNSSGSIELV |
| | (SEQ ID NO: 76) with 2 | KKGCWLDDFNCYDRTDCVATEENPQVYFCCCEGNFCNERFTHLPEAGGPEV |
| | additional aa at N-terminus | TYEPPPTAPT |
| 749 | Hybrid hu-ActRIIB-ECD | GEAETRECIYYNANWELERTNQSGLERCYGDKDKRRHCYASWRNSSGSIEL |
| | (SEQ ID NO: 76) with 3 | VKKGCWLDDFNCYDRTDCVATEENPQVYFCCCEGNFCNERFTHLPEAGGPE |
| | additional aa at N-terminus | VTYEPPPTAPT |
| 750 | Hybrid hu-ActRIIB-ECD | RGEAETRECIYYNANWELERTNQSGLERCYGDKDKRRHCYASWRNSSGSIE |
| | (SEQ ID NO: 76) with 4 | LVKKGCWLDDFNCYDRTDCVATEENPQVYFCCCEGNFCNERFTHLPEAGGP |
| | additional aa at N-terminus | EVTYEPPPTAPT |
| 751 | Hybrid hu-ActRIIB-ECD | GRGEAETRECIYYNANWELERTNQSGLERCYGDKDKRRHCYASWRNSSGSI |
| | (SEQ ID NO: 76) with 5 | ELVKKGCWLDDFNCYDRTDCVATEENPQVYFCCCEGNFCNERFTHLPEAGG |
| | additional aa at N-terminus | PEVTYEPPPTAPT |
| 752 | Hybrid hu-ActRIIB-ECD | SGRGEAETRECIYYNANWELERTNQSGLERCYGDKDKRRHCYASWRNSSGS |
| | (SEQ ID NO: 76) with 6 | IELVKKGCWLDDFNCYDRTDCVATEENPQVYFCCCEGNFCNERFTHLPEAG |
| | additional aa at N-terminus | GPEVTYEPPPTAPT |
| 753 | Hybrid hu-ActRIIB-ECD | AETRECIYYNANWELERTNQSGLERCYGDKDKRRHCYASWRNSSGSIEIVK |
| | (SEQ ID NO: 77) with 1 | KGCWLDDFNCYDRTDCVATEENPQVYFCCCEGNFCNERFTHLPEAGGPEVT |
| | additional aa at N-terminus | YEPPPTAPT |
| 754 | Hybrid hu-ActRIIB-ECD | EAETRECIYYNANWELERTNQSGLERCYGDKDKRRHCYASWRNSSGSIEIV |
| | (SEQ ID NO: 77) with 2 | KKGCWLDDFNCYDRTDCVATEENPQVYFCCCEGNFCNERFTHLPEAGGPEV |
| | additional aa at N-terminus | TYEPPPTAPT |
| 755 | Hybrid hu-ActRIIB-ECD | GEAETRECIYYNANWELERTNQSGLERCYGDKDKRRHCYASWRNSSGSIEI |
| | (SEQ ID NO: 77) with 3 | VKKGCWLDDFNCYDRTDCVATEENPQVYFCCCEGNFCNERFTHLPEAGGPE |
| | additional aa at N-terminus | VTYEPPPTAPT |
| 756 | Hybrid hu-ActRIIB-ECD | RGEAETRECIYYNANWELERTNQSGLERCYGDKDKRRHCYASWRNSSGSIEI |
| | (SEQ ID NO: 77) with 4 | VKKGCWLDDFNCYDRTDCVATEENPQVYFCCCEGNFCNERFTHLPEAGGPE |
| | additional aa at N-terminus | VTYEPPPTAPT |
| 757 | Hybrid hu-ActRIIB-ECD | GRGEAETRECIYYNANWELERTNQSGLERCYGDKDKRRHCYASWRNSSGSI |
| | (SEQ ID NO: 77) with 5 | EIVKKGCWLDDFNCYDRTDCVATEENPQVYFCCCEGNFCNERFTHLPEAGG |
| | additional aa at N-terminus | PEVTYEPPPTAPT |
| 758 | Hybrid hu-ActRIIB-ECD | SGRGEAETRECIYYNANWELERTNQSGLERCYGDKDKRRHCYASWRNSSGS |
| | (SEQ ID NO: 77) with 6 | IEIVKKGCWLDDFNCYDRTDCVATEENPQVYFCCCEGNFCNERFTHLPEAG |
| | additional aa at N-terminus | GPEVTYEPPPTAPT |
| 759 | Hybrid hu-ActRIIB-ECD | AETRECIYYNANWELERTNQSGLERCYGDKDKRRHCYASWRNSSGSIEIVK |
| | (SEQ ID NO: 78) with 1 | QGCWLDDFNCYDRTDCVATEENPQVYFCCCEGNFCNERFTHLPEAGGPEVT |
| | additional aa at N-terminus | YEPPPTAPT |
| 760 | Hybrid hu-ActRIIB-ECD | EAETRECIYYNANWELERTNQSGLERCYGDKDKRRHCYASWRNSSGSIEIV |
| | (SEQ ID NO: 78) with 2 | KQGCWLDDFNCYDRTDCVATEENPQVYFCCCEGNFCNERFTHLPEAGGPEV |
| | additional aa at N-terminus | TYEPPPTAPT |
| 761 | Hybrid hu-ActRIIB-ECD | GEAETRECIYYNANWELERTNQSGLERCYGDKDKRRHCYASWRNSSGSIEI |
| | (SEQ ID NO: 78) with 3 | VKQGCWLDDFNCYDRTDCVATEENPQVYFCCCEGNFCNERFTHLPEAGGPE |
| | additional aa at N-terminus | VTYEPPPTAPT |
| 762 | Hybrid hu-ActRIIB-ECD | RGEAETRECIYYNANWELERTNQSGLERCYGDKDKRRHCYASWRNSSGSIEI |
| | (SEQ ID NO: 78) with 4 | VKQGCWLDDFNCYDRTDCVATEENPQVYFCCCEGNFCNERFTHLPEAGGPE |
| | additional aa at N-terminus | VTYEPPPTAPT |
| 763 | Hybrid hu-ActRIIB-ECD | GRGEAETRECIYYNANWELERTNQSGLERCYGDKDKRRHCYASWRNSSGSI |
| | (SEQ ID NO: 78) with 5 | EIVKQGCWLDDFNCYDRTDCVATEENPQVYFCCCEGNFCNERFTHLPEAGG |
| | additional aa at N-terminus | PEVTYEPPPTAPT |
| 764 | Hybrid hu-ActRIIB-ECD | SGRGEAETRECIYYNANWELERTNQSGLERCYGDKDKRRHCYASWRNSSGS |
| | (SEQ ID NO: 78) with 6 | IEIVKQGCWLDDFNCYDRTDCVATEENPQVYFCCCEGNFCNERFTHLPEAG |
| | additional aa at N-terminus | GPEVTYEPPPTAPT |
| 765 | Hybrid hu-ActRIIB-ECD | AETRECIYYNANWELERTNQSGLERCEGEQDKRLHCYASWRNSSGTIELVK |
| | (SEQ ID NO: 79) with 1 | KGCWLDDFNCYDRTDCVATEENPQVYFCCCEGNFCNERFTHLPEAGGPEVT |
| | additional aa at N-terminus | YEPPPTAPT |
| 766 | Hybrid hu-ActRIIB-ECD | EAETRECIYYNANWELERTNQSGLERCEGEQDKRLHCYASWRNSSGTIELV |
| | (SEQ ID NO: 79) with 2 | KKGCWLDDFNCYDRTDCVATEENPQVYFCCCEGNFCNERFTHLPEAGGPEV |
| | additional aa at N-terminus | TYEPPPTAPT |
| 767 | Hybrid hu-ActRIIB-ECD | GEAETRECIYYNANWELERTNQSGLERCEGEQDKRLHCYASWRNSSGTIEL |
| | (SEQ ID NO: 79) with 3 | VKKGCWLDDFNCYDRTDCVATEENPQVYFCCCEGNFCNERFTHLPEAGGPE |
| | additional aa at N-terminus | VTYEPPPTAPT |
| 768 | Hybrid hu-ActRIIB-ECD | RGEAETRECIYYNANWELERTNQSGLERCEGEQDKRLHCYASWRNSSGTIE |
| | (SEQ ID NO: 79) with 4 | LVKKGCWLDDFNCYDRTDCVATEENPQVYFCCCEGNFCNERFTHLPEAGGP |
| | additional aa at N-terminus | EVTYEPPPTAPT |
| 769 | Hybrid hu-ActRIIB-ECD | GRGEAETRECIYYNANWELERTNQSGLERCEGEQDKRLHCYASWRNSSGTI |
| | (SEQ ID NO: 79) with 5 | ELVKKGCWLDDFNCYDRTDCVATEENPQVYFCCCEGNFCNERFTHLPEAGG |
| | additional aa at N-terminus | PEVTYEPPPTAPT |
| 770 | Hybrid hu-ActRIIB-ECD | SGRGEAETRECIYYNANWELERTNQSGLERCEGEQDKRLHCYASWRNSSGT |
| | (SEQ ID NO: 79) with 6 | IELVKKGCWLDDFNCYDRTDCVATEENPQVYFCCCEGNFCNERFTHLPEAG |
| | additional aa at N-terminus | GPEVTYEPPPTAPT |
| 771 | Hybrid hu-ActRIIB-ECD | AETRECIYYNANWELERTNQSGLERCEGEQDKRLHCYASWRNSSGTIELVK |
| | (SEQ ID NO: 80) with 1 | KGCWLDDFNCYDRQECVATEENPQVYFCCCEGNFCNEKFSYFPEMEVTQPT |
| | additional aa at N-terminus | SNPVTPKPP |
| 772 | Hybrid hu-ActRIIB-ECD | EAETRECIYYNANWELERTNQSGLERCEGEQDKRLHCYASWRNSSGTIELV |
| | (SEQ ID NO: 80) with 2 | KKGCWLDDFNCYDRQECVATEENPQVYFCCCEGNFCNEKFSYFPEMEVTQP |
| | additional aa at N-terminus | TSNPVTPKPP |
| 773 | Hybrid hu-ActRIIB-ECD | GEAETRECIYYNANWELERTNQSGLERCEGEQDKRLHCYASWRNSSGTIEL |
| | (SEQ ID NO: 80) with 3 | VKKGCWLDDFNCYDRQECVATEENPQVYFCCCEGNFCNEKFSYFPEMEVT |
| | additional aa at N-terminus | QPTSNPVTPKPP |
| 774 | Hybrid hu-ActRIIB-ECD | RGEAETRECIYYNANWELERTNQSGLERCEGEQDKRLHCYASWRNSSGTIE |
| | (SEQ ID NO: 80) with 4 | LVKKGCWLDDFNCYDRQECVATEENPQVYFCCCEGNFCNEKFSYFPEMEVT |
| | additional aa at N-terminus | QPTSNPVTPKPP |
| 775 | Hybrid hu-ActRIIB-ECD | GRGEAETRECIYYNANWELERTNQSGLERCEGEQDKRLHCYASWRNSSGTI |
| | (SEQ ID NO: 80) with 5 | ELVKKGCWLDDFNCYDRQECVATEENPQVYFCCCEGNFCNEKFSYFPEMEV |
| | additional aa at N-terminus | TQPTSNPVTPKPP |
| 776 | Hybrid hu-ActRIIB-ECD | SGRGEAETRECIYYNANWELERTNQSGLERCEGEQDKRLHCYASWRNSSGT |
| | (SEQ ID NO: 80) with 6 | IELVKKGCWLDDFNCYDRQECVATEENPQVYFCCCEGNFCNEKFSYFPEME |
| | additional aa at N-terminus | VTQPTSNPVTPKPP |
| 777 | Hybrid hu-ActRIIB-ECD | AETRECIYYNANWELERTNQSGLERCEGEQDKRLHCYASWRNSSGTIELVK |
| | (SEQ ID NO: 81) with 1 | KGCWLDDFNCYDRQECVATEENPQVYFCCCEGNFCNEKFSYFPQMEVTQPT |
| | additional aa at N-terminus | SNPVTPKPP |
| 778 | Hybrid hu-ActRIIB-ECD | EAETRECIYYNANWELERTNQSGLERCEGEQDKRLHCYASWRNSSGTIELV |
| | (SEQ ID NO: 81) with 2 | KKGCWLDDFNCYDRQECVATEENPQVYFCCCEGNFCNEKFSYFPQMEVTQ |
| | additional aa at N-terminus | PTSNPVTPKPP |
| 779 | Hybrid hu-ActRIIB-ECD | GEAETRECIYYNANWELERTNQSGLERCEGEQDKRLHCYASWRNSSGTIEL |
| | (SEQ ID NO: 81) with 3 | VKKGCWLDDFNCYDRQECVATEENPQVYFCCCEGNFCNEKFSYFPQMEVT |
| | additional aa at N-terminus | QPTSNPVTPKPP |
| 780 | Hybrid hu-ActRIIB-ECD | RGEAETRECIYYNANWELERTNQSGLERCEGEQDKRLHCYASWRNSSGTIE |
| | (SEQ ID NO: 81) with 4 | LVKKGCWLDDFNCYDRQECVATEENPQVYFCCCEGNFCNEKFSYFPQMEV |
| | additional aa at N-terminus | TQPTSNPVTPKPP |
| 781 | Hybrid hu-ActRIIB-ECD | GRGEAETRECIYYNANWELERTNQSGLERCEGEQDKRLHCYASWRNSSGTI |
| | (SEQ ID NO: 81) with 5 | ELVKKGCWLDDFNCYDRQECVATEENPQVYFCCCEGNFCNEKFSYFPQME |
| | additional aa at N-terminus | VTQPTSNPVTPKPP |
| 782 | Hybrid hu-ActRIIB-ECD | SGRGEAETRECIYYNANWELERTNQSGLERCEGEQDKRLHCYASWRNSSGT |
| | (SEQ ID NO: 81) with 6 | IELVKKGCWLDDFNCYDRQECVATEENPQVYFCCCEGNFCNEKFSYFPQME |
| | additional aa at N-terminus | VTQPTSNPVTPKPP |
| 783 | Hybrid hu-ActRIIB-ECD | AETRECIYYNANWELERTNQSGLERCYGDKDKRRHCYASWRNSSGTIELVK |
| | (SEQ ID NO: 82) with 1 | KGCWLDDINCYDRTDCVEKKDSPEVYFCCCEGNFCNEKFSYFPEMEVTQPT |
| | additional aa at N-terminus | SNPVTPKPP |
| 784 | Hybrid hu-ActRIIB-ECD | EAETRECIYYNANWELERTNQSGLERCYGDKDKRRHCYASWRNSSGTIELV |
| | (SEQ ID NO: 82) with 2 | KKGCWLDDINCYDRTDCVEKKDSPEVYFCCCEGNFCNEKFSYFPEMEVTQP |
| | additional aa at N-terminus | TSNPVTPKPP |
| 785 | Hybrid hu-ActRIIB-ECD | GEAETRECIYYNANWELERTNQSGLERCYGDKDKRRHCYASWRNSSGTIEL |
| | (SEQ ID NO: 82) with 3 | VKKGCWLDDINCYDRTDCVEKKDSPEVYFCCCEGNFCNEKFSYFPEMEVTQ |
| | additional aa at N-terminus | PTSNPVTPKPP |
| 786 | Hybrid hu-ActRIIB-ECD | RGEAETRECIYYNANWELERTNQSGLERCYGDKDKRRHCYASWRNSSGTIE |
| | (SEQ ID NO: 82) with 4 | LVKKGCWLDDINCYDRTDCVEKKDSPEVYFCCCEGNFCNEKFSYFPEMEVT |
| | additional aa at N-terminus | QPTSNPVTPKPP |
| 787 | Hybrid hu-ActRIIB-ECD | GRGEAETRECIYYNANWELERTNQSGLERCYGDKDKRRHCYASWRNSSGTI |
| | (SEQ ID NO: 82) with 5 | ELVKKGCWLDDINCYDRTDCVEKKDSPEVYFCCCEGNFCNEKFSYFPEMEV |
| | additional aa at N-terminus | TQPTSNPVTPKPP |
| 788 | Hybrid hu-ActRIIB-ECD | SGRGEAETRECIYYNANWELERTNQSGLERCYGDKDKRRHCYASWRNSSG |
| | (SEQ ID NO: 82) with 6 | TIELVKKGCWLDDINCYDRTDCVEKKDSPEVYFCCCEGNFCNEKFSYFPEME |
| | additional aa at N-terminus | VTQPTSNPVTPKPP |
| 789 | Hybrid hu-ActRIIB-ECD | AETRECIYYNANWELERTNQSGLERCYGDKDKRRHCYASWRNSSGTIELVK |
| | (SEQ ID NO: 83) with 1 | KGCWLDDINCYDRTDCVEKKDSPEVYFCCCEGNFCNEKFSYFPQMEVTQPT |
| | additional aa at N-terminus | SNPVTPKPP |
| 790 | Hybrid hu-ActRIIB-ECD | EAETRECIYYNANWELERTNQSGLERCYGDKDKRRHCYASWRNSSGTIELV |
| | (SEQ ID NO: 83) with 2 | KKGCWLDDINCYDRTDCVEKKDSPEVYFCCCEGNFCNEKFSYFPQMEVTQP |
| | additional aa at N-terminus | TSNPVTPKPP |
| 791 | Hybrid hu-ActRIIB-ECD | GEAETRECIYYNANWELERTNQSGLERCYGDKDKRRHCYASWRNSSGTIEL |
| | (SEQ ID NO: 83) with 3 | VKKGCWLDDINCYDRTDCVEKKDSPEVYFCCCEGNFCNEKFSYFPQMEVTQ |
| | additional aa at N-terminus | PTSNPVTPKPP |
| 792 | Hybrid hu-ActRIIB-ECD | RGEAETRECIYYNANWELERTNQSGLERCYGDKDKRRHCYASWRNSSGTIE |
| | (SEQ ID NO: 83) with 4 | LVKKGCWLDDINCYDRTDCVEKKDSPEVYFCCCEGNFCNEKFSYFPQMEVT |
| | additional aa at N-terminus | QPTSNPVTPKPP |
| 793 | Hybrid hu-ActRIIB-ECD | GRGEAETRECIYYNANWELERTNQSGLERCYGDKDKRRHCYASWRNSSGTI |
| | (SEQ ID NO: 83) with 5 | ELVKKGCWLDDINCYDRTDCVEKKDSPEVYFCCCEGNFCNEKFSYFPQMEV |
| | additional aa at N-terminus | TQPTSNPVTPKPP |
| 794 | Hybrid hu-ActRIIB-ECD | SGRGEAETRECIYYNANWELERTNQSGLERCYGDKDKRRHCYASWRNSSG |
| | (SEQ ID NO: 83) with 6 | TIELVKKGCWLDDINCYDRTDCVEKKDSPEVYFCCCEGNFCNEKFSYFPQM |
| | additional aa at N-terminus | EVTQPTSNPVTPKPP |
| 795 | Hybrid hu-ActRIIB-ECD | AETRECIYYNANWELERTNQSGLERCYGDKDKRRHCYASWRNSSGTIELVK |
| | (SEQ ID NO: 84) with 1 | KGCWLDDFNCYDRQECVATEENPQVYFCCCEGNFCNEKFSYFPQMEVTQPT |
| | additional aa at N-terminus | SNPVTPKPP |
| 796 | Hybrid hu-ActRIIB-ECD | EAETRECIYYNANWELERTNQSGLERCYGDKDKRRHCYASWRNSSGTIELV |
| | (SEQ ID NO: 84) with 2 | KKGCWLDDFNCYDRQECVATEENPQVYFCCCEGNFCNEKFSYFPQMEVTQ |
| | additional aa at N-terminus | PTSNPVTPKPP |
| 797 | Hybrid hu-ActRIIB-ECD | GEAETRECIYYNANWELERTNQSGLERCYGDKDKRRHCYASWRNSSGTIEL |
| | (SEQ ID NO: 84) with 3 | VKKGCWLDDFNCYDRQECVATEENPQVYFCCCEGNFCNEKFSYFPQMEVT |
| | additional aa at N-terminus | QPTSNPVTPKPP |
| 798 | Hybrid hu-ActRIIB-ECD | RGEAETRECIYYNANWELERTNQSGLERCYGDKDKRRHCYASWRNSSGTIE |
| | (SEQ ID NO: 84) with 4 | LVKKGCWLDDFNCYDRQECVATEENPQVYFCCCEGNFCNEKFSYFPQMEV |
| | additional aa at N-terminus | TQPTSNPVTPKPP |
| 799 | Hybrid hu-ActRIIB-ECD | GRGEAETRECIYYNANWELERTNQSGLERCYGDKDKRRHCYASWRNSSGTI |
| | (SEQ ID NO: 84) with 5 | ELVKKGCWLDDFNCYDRQECVATEENPQVYFCCCEGNFCNEKFSYFPQME |
| | additional aa at N-terminus | VTQPTSNPVTPKPP |
| 800 | Hybrid hu-ActRIIB-ECD | SGRGEAETRECIYYNANWELERTNQSGLERCYGDKDKRRHCYASWRNSSG |
| | (SEQ ID NO: 84) with 6 | TIELVKKGCWLDDFNCYDRQECVATEENPQVYFCCCEGNFCNEKFSYFPQM |
| | additional aa at N-terminus | EVTQPTSNPVTPKPP |
| 801 | Hybrid hu-ActRIIB-ECD | AETRECIYYNANWELERTNQSGLERCYGDKDKRRHCYASWRNSSGTIELVK |
| | (SEQ ID NO: 85) with 1 | QGCWLDDFNCYDRQECVATEENPQVYFCCCEGNFCNEKFSYFPQMEVTQPT |
| | additional aa at N-terminus | SNPVTPKPP |
| 802 | Hybrid hu-ActRIIB-ECD | EAETRECIYYNANWELERTNQSGLERCYGDKDKRRHCYASWRNSSGTIELV |
| | (SEQ ID NO: 85) with 2 | KQGCWLDDFNCYDRQECVATEENPQVYFCCCEGNFCNEKFSYFPQMEVTQ |
| | additional aa at N-terminus | PTSNPVTPKPP |
| 803 | Hybrid hu-ActRIIB-ECD | GEAETRECIYYNANWELERTNQSGLERCYGDKDKRRHCYASWRNSSGTIEL |
| | (SEQ ID NO: 85) with 3 | VKQGCWLDDFNCYDRQECVATEENPQVYFCCCEGNFCNEKFSYFPQMEVT |
| | additional aa at N-terminus | QPTSNPVTPKPP |
| 804 | Hybrid hu-ActRIIB-ECD | RGEAETRECIYYNANWELERTNQSGLERCYGDKDKRRHCYASWRNSSGTIE |
| | (SEQ ID NO: 85) with 4 | LVKQGCWLDDFNCYDRQECVATEENPQVYFCCCEGNFCNEKFSYFPQMEV |
| | additional aa at N-terminus | TQPTSNPVTPKPP |
| 805 | Hybrid hu-ActRIIB-ECD | GRGEAETRECIYYNANWELERTNQSGLERCYGDKDKRRHCYASWRNSSGTI |
| | (SEQ ID NO: 85) with 5 | ELVKQGCWLDDFNCYDRQECVATEENPQVYFCCCEGNFCNEKFSYFPQME |
| | additional aa at N-terminus | VTQPTSNPVTPKPP |
| 806 | Hybrid hu-ActRIIB-ECD | SGRGEAETRECIYYNANWELERTNQSGLERCYGDKDKRRHCYASWRNSSG |
| | (SEQ ID NO: 85) with 6 | TIELVKQGCWLDDFNCYDRQECVATEENPQVYFCCCEGNFCNEKFSYFPQM |
| | additional aa at N-terminus | EVTQPTSNPVTPKPP |
| 807 | Hybrid hu-ActRIIB-ECD | AETRECIYYNANWELERTNQSGLERCYGDKDKRRHCYASWRNSSGTIEIVK |
| | (SEQ ID NO: 86) with 1 | KGCWLDDFNCYDRQECVATKENPQVYFCCCEGNFCNEKFSYFPQMEVTQP |
| | additional aa at N-terminus | TSNPVTPKPP |
| 808 | Hybrid hu-ActRIIB-ECD | EAETRECIYYNANWELERTNQSGLERCYGDKDKRRHCYASWRNSSGTIEIV |
| | (SEQ ID NO: 86) with 2 | KKGCWLDDFNCYDRQECVATKENPQVYFCCCEGNFCNEKFSYFPQMEVTQ |
| | additional aa at N-terminus | PTSNPVTPKPP |
| 809 | Hybrid hu-ActRIIB-ECD | GEAETRECIYYNANWELERTNQSGLERCYGDKDKRRHCYASWRNSSGTIEI |
| | (SEQ ID NO: 86) with 3 | VKKGCWLDDFNCYDRQECVATKENPQVYFCCCEGNFCNEKFSYFPQMEVT |
| | additional aa at N-terminus | QPTSNPVTPKPP |
| 810 | Hybrid hu-ActRIIB-ECD | RGEAETRECIYYNANWELERTNQSGLERCYGDKDKRRHCYASWRNSSGTIE |
| | (SEQ ID NO: 86) with 4 | IVKKGCWLDDFNCYDRQECVATKENPQVYFCCCEGNFCNEKFSYFPQMEVT |
| | additional aa at N-terminus | QPTSNPVTPKPP |
| 811 | Hybrid hu-ActRIIB-ECD | GRGEAETRECIYYNANWELERTNQSGLERCYGDKDKRRHCYASWRNSSGTI |
| | (SEQ ID NO: 86) with 5 | EIVKKGCWLDDFNCYDRQECVATKENPQVYFCCCEGNFCNEKFSYFPQMEV |
| | additional aa at N-terminus | TQPTSNPVTPKPP |
| 812 | Hybrid hu-ActRIIB-ECD | SGRGEAETRECIYYNANWELERTNQSGLERCYGDKDKRRHCYASWRNSSG |
| | (SEQ ID NO: 86) with 6 | TIEIVKKGCWLDDFNCYDRQECVATKENPQVYFCCCEGNFCNEKFSYFPQM |
| | additional aa at N-terminus | EVTQPTSNPVTPKPP |
| 813 | Hybrid hu-ActRIIB-ECD | AETRECIYYNANWELERTNQSGLERCYGDKDKRRHCYASWRNSSGSIELVK |
| | (SEQ ID NO: 87) with 1 | KGCWLDDFNCYDRQECVATKENPQVYFCCCEGNFCNEKFSYFPQMEVTQP |
| | additional aa at N-terminus | TSNPVTPKPP |
| 814 | Hybrid hu-ActRIIB-ECD | EAETRECIYYNANWELERTNQSGLERCYGDKDKRRHCYASWRNSSGSIELV |
| | (SEQ ID NO: 87) with 2 | KKGCWLDDFNCYDRQECVATKENPQVYFCCCEGNFCNEKFSYFPQMEVTQ |
| | additional aa at N-terminus | PTSNPVTPKPP |
| 815 | Hybrid hu-ActRIIB-ECD | GEAETRECIYYNANWELERTNQSGLERCYGDKDKRRHCYASWRNSSGSIEL |
| | (SEQ ID NO: 87) with 3 | VKKGCWLDDFNCYDRQECVATKENPQVYFCCCEGNFCNEKFSYFPQMEVT |
| | additional aa at N-terminus | QPTSNPVTPKPP |
| 816 | Hybrid hu-ActRIIB-ECD | RGEAETRECIYYNANWELERTNQSGLERCYGDKDKRRHCYASWRNSSGSIE |
| | (SEQ ID NO: 87) with 4 | LVKKGCWLDDFNCYDRQECVATKENPQVYFCCCEGNFCNEKFSYFPQMEV |
| | additional aa at N-terminus | TQPTSNPVTPKPP |
| 817 | Hybrid hu-ActRIIB-ECD | GRGEAETRECIYYNANWELERTNQSGLERCYGDKDKRRHCYASWRNSSGSI |
| | (SEQ ID NO: 87) with 5 | ELVKKGCWLDDFNCYDRQECVATKENPQVYFCCCEGNFCNEKFSYFPQME |
| | additional aa at N-terminus | VTQPTSNPVTPKPP |
| 818 | Hybrid hu-ActRIIB-ECD | SGRGEAETRECIYYNANWELERTNQSGLERCYGDKDKRRHCYASWRNSSGS |
| | (SEQ ID NO: 87) with 6 | IELVKKGCWLDDFNCYDRQECVATKENPQVYFCCCEGNFCNEKFSYFPQME |
| | additional aa at N-terminus | VTQPTSNPVTPKPP |
| 819 | Hybrid hu-ActRIIB-ECD | AETRECIYYNANWELERTNQSGLERCEGEQDKRLHCYASWRNSSGSIELVK |
| | (SEQ ID NO: 88) with 1 | KGCWLDDFNCYDRQECVATEENPQVYFCCCEGNFCNEKFSYFPQMEVTQPT |
| | additional aa at N-terminus | SNPVTPKPP |
| 820 | Hybrid hu-ActRIIB-ECD | EAETRECIYYNANWELERTNQSGLERCEGEQDKRLHCYASWRNSSGSIELV |
| | (SEQ ID NO: 88) with 2 | KKGCWLDDFNCYDRQECVATEENPQVYFCCCEGNFCNEKFSYFPQMEVTQ |
| | additional aa at N-terminus | PTSNPVTPKPP |
| 821 | Hybrid hu-ActRIIB-ECD | GEAETRECIYYNANWELERTNQSGLERCEGEQDKRLHCYASWRNSSGSIEL |
| | (SEQ ID NO: 88) with 3 | VKKGCWLDDFNCYDRQECVATEENPQVYFCCCEGNFCNEKFSYFPQMEVT |
| | additional aa at N-terminus | QPTSNPVTPKPP |
| 822 | Hybrid hu-ActRIIB-ECD | RGEAETRECIYYNANWELERTNQSGLERCEGEQDKRLHCYASWRNSSGSIEL |
| | (SEQ ID NO: 88) with 4 | VKKGCWLDDFNCYDRQECVATEENPQVYFCCCEGNFCNEKFSYFPQMEVT |
| | additional aa at N-terminus | QPTSNPVTPKPP |
| 823 | Hybrid hu-ActRIIB-ECD | GRGEAETRECIYYNANWELERTNQSGLERCEGEQDKRLHCYASWRNSSGSI |
| | (SEQ ID NO: 88) with 5 | ELVKKGCWLDDFNCYDRQECVATEENPQVYFCCCEGNFCNEKFSYFPQME |
| | additional aa at N-terminus | VTQPTSNPVTPKPP |
| 824 | Hybrid hu-ActRIIB-ECD | SGRGEAETRECIYYNANWELERTNQSGLERCEGEQDKRLHCYASWRNSSGS |
| | (SEQ ID NO: 88) with 6 | IELVKKGCWLDDFNCYDRQECVATEENPQVYFCCCEGNFCNEKFSYFPQME |
| | additional aa at N-terminus | VTQPTSNPVTPKPP |
| 825 | Hybrid hu-ActRIIB-ECD | AETRECIYYNANWELERTNQSGLERCEGEQDKRLHCYASWRNSSGTIEIVKK |
| | (SEQ ID NO: 89) with 1 | GCWLDDFNCYDRQECVATEENPQVYFCCCEGNFCNEKFSYFPQMEVTQPTS |
| | additional aa at N-terminus | NPVTPKPP |
| 826 | Hybrid hu-ActRIIB-ECD | EAETRECIYYNANWELERTNQSGLERCEGEQDKRLHCYASWRNSSGTIEIVK |
| | (SEQ ID NO: 89) with 2 | KGCWLDDFNCYDRQECVATEENPQVYFCCCEGNFCNEKFSYFPQMEVTQPT |
| | additional aa at N-terminus | SNPVTPKPP |
| 827 | Hybrid hu-ActRIIB-ECD | GEAETRECIYYNANWELERTNQSGLERCEGEQDKRLHCYASWRNSSGTIEIV |
| | (SEQ ID NO: 89) with 3 | KKGCWLDDFNCYDRQECVATEENPQVYFCCCEGNFCNEKFSYFPQMEVTQ |
| | additional aa at N-terminus | PTSNPVTPKPP |
| 828 | Hybrid hu-ActRIIB-ECD | RGEAETRECIYYNANWELERTNQSGLERCEGEQDKRLHCYASWRNSSGTIEI |
| | (SEQ ID NO: 89) with 4 | VKKGCWLDDFNCYDRQECVATEENPQVYFCCCEGNFCNEKFSYFPQMEVT |
| | additional aa at N-terminus | QPTSNPVTPKPP |
| 829 | Hybrid hu-ActRIIB-ECD | GRGEAETRECIYYNANWELERTNQSGLERCEGEQDKRLHCYASWRNSSGTI |
| | (SEQ ID NO: 89) with 5 | EIVKKGCWLDDFNCYDRQECVATEENPQVYFCCCEGNFCNEKFSYFPQMEV |
| | additional aa at N-terminus | TQPTSNPVTPKPP |
| 830 | Hybrid hu-ActRIIB-ECD | SGRGEAETRECIYYNANWELERTNQSGLERCEGEQDKRLHCYASWRNSSGT |
| | (SEQ ID NO: 89) with 6 | IEIVKKGCWLDDFNCYDRQECVATEENPQVYFCCCEGNFCNEKFSYFPQME |
| | additional aa at N-terminus | VTQPTSNPVTPKPP |
| 831 | Hybrid hu-ActRIIB-ECD | AETRECIYYNANWELERTNQSGLERCEGEQDKRLHCYASWRNSSGTIELVK |
| | (SEQ ID NO: 90) with 1 | QGCWLDDFNCYDRQECVATEENPQVYFCCCEGNFCNEKFSYFPQMEVTQPT |
| | additional aa at N-terminus | SNPVTPKPP |
| 832 | Hybrid hu-ActRIIB-ECD | EAETRECIYYNANWELERTNQSGLERCEGEQDKRLHCYASWRNSSGTIELV |
| | (SEQ ID NO: 90) with 2 | KQGCWLDDFNCYDRQECVATEENPQVYFCCCEGNFCNEKFSYFPQMEVTQ |
| | additional aa at N-terminus | PTSNPVTPKPP |
| 833 | Hybrid hu-ActRIIB-ECD | GEAETRECIYYNANWELERTNQSGLERCEGEQDKRLHCYASWRNSSGTIEL |
| | (SEQ ID NO: 90) with 3 | VKQGCWLDDFNCYDRQECVATEENPQVYFCCCEGNFCNEKFSYFPQMEVT |
| | additional aa at N-terminus | QPTSNPVTPKPP |
| 834 | Hybrid hu-ActRIIB-ECD | RGEAETRECIYYNANWELERTNQSGLERCEGEQDKRLHCYASWRNSSGTIE |
| | (SEQ ID NO: 90) with 4 | LVKQGCWLDDFNCYDRQECVATEENPQVYFCCCEGNFCNEKFSYFPQMEV |
| | additional aa at N-terminus | TQPTSNPVTPKPP |
| 835 | Hybrid hu-ActRIIB-ECD | GRGEAETRECIYYNANWELERTNQSGLERCEGEQDKRLHCYASWRNSSGTI |
| | (SEQ ID NO: 90) with 5 | ELVKQGCWLDDFNCYDRQECVATEENPQVYFCCCEGNFCNEKFSYFPQME |
| | additional aa at N-terminus | VTQPTSNPVTPKPP |
| 836 | Hybrid hu-ActRIIB-ECD | SGRGEAETRECIYYNANWELERTNQSGLERCEGEQDKRLHCYASWRNSSGT |
| | (SEQ ID NO: 90) with 6 | IELVKQGCWLDDFNCYDRQECVATEENPQVYFCCCEGNFCNEKFSYFPQME |
| | additional aa at N-terminus | VTQPTSNPVTPKPP |
| 837 | Hybrid hu-ActRIIB-ECD | AETRECIYYNANWELERTNQSGLERCEGEQDKRLHCYASWRNSSGTIELVK |
| | (SEQ ID NO: 91) with 1 | KGCWLDDFNCYDRQECVETEENPQVYFCCCEGNFCNEKFSYFPQMEVTQPT |
| | additional aa at N-terminus | SNPVTPKPP |
| 838 | Hybrid hu-ActRIIB-ECD | EAETRECIYYNANWELERTNQSGLERCEGEQDKRLHCYASWRNSSGTIELV |
| | (SEQ ID NO: 91) with 2 | KKGCWLDDFNCYDRQECVETEENPQVYFCCCEGNFCNEKFSYFPQMEVTQP |
| | additional aa at N-terminus | TSNPVTPKPP |
| 839 | Hybrid hu-ActRIIB-ECD | GEAETRECIYYNANWELERTNQSGLERCEGEQDKRLHCYASWRNSSGTIEL |
| | (SEQ ID NO: 91) with 3 | VKKGCWLDDFNCYDRQECVETEENPQVYFCCCEGNFCNEKFSYFPQMEVT |
| | additional aa at N-terminus | QPTSNPVTPKPP |
| 840 | Hybrid hu-ActRIIB-ECD | RGEAETRECIYYNANWELERTNQSGLERCEGEQDKRLHCYASWRNSSGTIE |
| | (SEQ ID NO: 91) with 4 | LVKKGCWLDDFNCYDRQECVETEENPQVYFCCCEGNFCNEKFSYFPQMEVT |
| | additional aa at N-terminus | QPTSNPVTPKPP |
| 841 | Hybrid hu-ActRIIB-ECD | GRGEAETRECIYYNANWELERTNQSGLERCEGEQDKRLHCYASWRNSSGTI |
| | (SEQ ID NO: 91) with 5 | ELVKKGCWLDDFNCYDRQECVETEENPQVYFCCCEGNFCNEKFSYFPQMEV |
| | additional aa at N-terminus | TQPTSNPVTPKPP |
| 842 | Hybrid hu-ActRIIB-ECD | SGRGEAETRECIYYNANWELERTNQSGLERCEGEQDKRLHCYASWRNSSGT |
| | (SEQ ID NO: 91) with 6 | IELVKKGCWLDDFNCYDRQECVETEENPQVYFCCCEGNFCNEKFSYFPQME |
| | additional aa at N-terminus | VTQPTSNPVTPKPP |
| 843 | Hybrid hu-ActRIIB-ECD | AETRECIYYNANWELERTNQSGLERCEGEQDKRLHCYASWRNSSGTIELVK |
| | (SEQ ID NO: 92) with 1 | KGCWLDDFNCYDRQECVAKEENPQVYFCCCEGNFCNEKFSYFPQMEVTQP |
| | additional aa at N-terminus | TSNPVTPKPP |
| 844 | Hybrid hu-ActRIIB-ECD | EAETRECIYYNANWELERTNQSGLERCEGEQDKRLHCYASWRNSSGTIELV |
| | (SEQ ID NO: 92) with 2 | KKGCWLDDFNCYDRQECVAKEENPQVYFCCCEGNFCNEKFSYFPQMEVTQ |
| | additional aa at N-terminus | PTSNPVTPKPP |
| 845 | Hybrid hu-ActRIIB-ECD | GEAETRECIYYNANWELERTNQSGLERCEGEQDKRLHCYASWRNSSGTIEL |
| | (SEQ ID NO: 92) with 3 | VKKGCWLDDFNCYDRQECVAKEENPQVYFCCCEGNFCNEKFSYFPQMEVT |
| | additional aa at N-terminus | QPTSNPVTPKPP |
| 846 | Hybrid hu-ActRIIB-ECD | RGEAETRECIYYNANWELERTNQSGLERCEGEQDKRLHCYASWRNSSGTIE |
| | (SEQ ID NO: 92) with 4 | LVKKGCWLDDFNCYDRQECVAKEENPQVYFCCCEGNFCNEKFSYFPQMEV |
| | additional aa at N-terminus | TQPTSNPVTPKPP |
| 847 | Hybrid hu-ActRIIB-ECD | GRGEAETRECIYYNANWELERTNQSGLERCEGEQDKRLHCYASWRNSSGTI |
| | (SEQ ID NO: 92) with 5 | ELVKKGCWLDDFNCYDRQECVAKEENPQVYFCCCEGNFCNEKFSYFPQME |
| | additional aa at N-terminus | VTQPTSNPVTPKPP |
| 848 | Hybrid hu-ActRIIB-ECD | SGRGEAETRECIYYNANWELERTNQSGLERCEGEQDKRLHCYASWRNSSGT |
| | (SEQ ID NO: 92) with 6 | IELVKKGCWLDDFNCYDRQECVAKEENPQVYFCCCEGNFCNEKFSYFPQME |
| | additional aa at N-terminus | VTQPTSNPVTPKPP |
| 849 | Hybrid hu-ActRIIB-ECD | AETRECIYYNANWELERTNQSGLERCEGEQDKRLHCYASWRNSSGTIELVK |
| | (SEQ ID NO: 93) with 1 | KGCWLDDFNCYDRQECVATKENPQVYFCCCEGNFCNEKFSYFPQMEVTQP |
| | additional aa at N-terminus | TSNPVTPKPP |
| 850 | Hybrid hu-ActRIIB-ECD | EAETRECIYYNANWELERTNQSGLERCEGEQDKRLHCYASWRNSSGTIELV |
| | (SEQ ID NO: 93) with 2 | KKGCWLDDFNCYDRQECVATKENPQVYFCCCEGNFCNEKFSYFPQMEVTQ |
| | additional aa at N-terminus | PTSNPVTPKPP |
| 851 | Hybrid hu-ActRIIB-ECD | GEAETRECIYYNANWELERTNQSGLERCEGEQDKRLHCYASWRNSSGTIEL |
| | (SEQ ID NO: 93) with 3 | VKKGCWLDDFNCYDRQECVATKENPQVYFCCCEGNFCNEKFSYFPQMEVT |
| | additional aa at N-terminus | QPTSNPVTPKPP |
| 852 | Hybrid hu-ActRIIB-ECD | RGEAETRECIYYNANWELERTNQSGLERCEGEQDKRLHCYASWRNSSGTIE |
| | (SEQ ID NO: 93) with 4 | LVKKGCWLDDFNCYDRQECVATKENPQVYFCCCEGNFCNEKFSYFPQMEV |
| | additional aa at N-terminus | TQPTSNPVTPKPP |
| 853 | Hybrid hu-ActRIIB-ECD | GRGEAETRECIYYNANWELERTNQSGLERCEGEQDKRLHCYASWRNSSGTI |
| | (SEQ ID NO: 93) with 5 | ELVKKGCWLDDFNCYDRQECVATKENPQVYFCCCEGNFCNEKFSYFPQME |
| | additional aa at N-terminus | VTQPTSNPVTPKPP |
| 854 | Hybrid hu-ActRIIB-ECD | SGRGEAETRECIYYNANWELERTNQSGLERCEGEQDKRLHCYASWRNSSGT |
| | (SEQ ID NO: 93) with 6 | IELVKKGCWLDDFNCYDRQECVATKENPQVYFCCCEGNFCNEKFSYFPQME |
| | additional aa at N-terminus | VTQPTSNPVTPKPP |
| 855 | Hybrid hu-ActRIIB-ECD | AETRECIYYNANWELERTNQSGLERCEGEQDKRLHCYASWRNSSGTIELVK |
| | (SEQ ID NO: 94) with 1 | KGCWLDDFNCYDRQECVATEDNPQVYFCCCEGNFCNEKFSYFPQMEVTQP |
| | additional aa at N-terminus | TSNPVTPKPP |
| 856 | Hybrid hu-ActRIIB-ECD | EAETRECIYYNANWELERTNQSGLERCEGEQDKRLHCYASWRNSSGTIELV |
| | (SEQ ID NO: 94) with 2 | KKGCWLDDFNCYDRQECVATEDNPQVYFCCCEGNFCNEKFSYFPQMEVTQ |
| | additional aa at N-terminus | PTSNPVTPKPP |
| 857 | Hybrid hu-ActRIIB-ECD | GEAETRECIYYNANWELERTNQSGLERCEGEQDKRLHCYASWRNSSGTIEL |
| | (SEQ ID NO: 94) with 3 | VKKGCWLDDFNCYDRQECVATEDNPQVYFCCCEGNFCNEKFSYFPQMEVT |
| | additional aa at N-terminus | QPTSNPVTPKPP |
| 858 | Hybrid hu-ActRIIB-ECD | RGEAETRECIYYNANWELERTNQSGLERCEGEQDKRLHCYASWRNSSGTIE |
| | (SEQ ID NO: 94) with 4 | LVKKGCWLDDFNCYDRQECVATEDNPQVYFCCCEGNFCNEKFSYFPQMEV |
| | additional aa at N-terminus | TQPTSNPVTPKPP |
| 859 | Hybrid hu-ActRIIB-ECD | GRGEAETRECIYYNANWELERTNQSGLERCEGEQDKRLHCYASWRNSSGTI |
| | (SEQ ID NO: 94) with 5 | ELVKKGCWLDDFNCYDRQECVATEDNPQVYFCCCEGNFCNEKFSYFPQME |
| | additional aa at N-terminus | VTQPTSNPVTPKPP |
| 860 | Hybrid hu-ActRIIB-ECD | SGRGEAETRECIYYNANWELERTNQSGLERCEGEQDKRLHCYASWRNSSGT |
| | (SEQ ID NO: 94) with 6 | IELVKKGCWLDDFNCYDRQECVATEDNPQVYFCCCEGNFCNEKFSYFPQME |
| | additional aa at N-terminus | VTQPTSNPVTPKPP |
| 861 | Hybrid hu-ActRIIB-ECD | AETRECIYYNANWELERTNQSGLERCEGEQDKRLHCYASWRNSSGTIELVK |
| | (SEQ ID NO: 95) with 1 | KGCWLDDFNCYDRQECVATEESPQVYFCCCEGNFCNEKFSYFPQMEVTQPT |
| | additional aa at N-terminus | SNPVTPKPP |
| 862 | Hybrid hu-ActRIIB-ECD | EAETRECIYYNANWELERTNQSGLERCEGEQDKRLHCYASWRNSSGTIELV |
| | (SEQ ID NO: 95) with 2 | KKGCWLDDFNCYDRQECVATEESPQVYFCCCEGNFCNEKFSYFPQMEVTQP |
| | additional aa at N-terminus | TSNPVTPKPP |
| 863 | Hybrid hu-ActRIIB-ECD | GEAETRECIYYNANWELERTNQSGLERCEGEQDKRLHCYASWRNSSGTIEL |
| | (SEQ ID NO: 95) with 3 | VKKGCWLDDFNCYDRQECVATEESPQVYFCCCEGNFCNEKFSYFPQMEVT |
| | additional aa at N-terminus | QPTSNPVTPKPP |
| 864 | Hybrid hu-ActRIIB-ECD | RGEAETRECIYYNANWELERTNQSGLERCEGEQDKRLHCYASWRNSSGTIE |
| | (SEQ ID NO: 95) with 4 | LVKKGCWLDDFNCYDRQECVATEESPQVYFCCCEGNFCNEKFSYFPQMEVT |
| | additional aa at N-terminus | QPTSNPVTPKPP |
| 865 | Hybrid hu-ActRIIB-ECD | GRGEAETRECIYYNANWELERTNQSGLERCEGEQDKRLHCYASWRNSSGTI |
| | (SEQ ID NO: 95) with 5 | ELVKKGCWLDDFNCYDRQECVATEESPQVYFCCCEGNFCNEKFSYFPQMEV |
| | additional aa at N-terminus | TQPTSNPVTPKPP |
| 866 | Hybrid hu-ActRIIB-ECD | SGRGEAETRECIYYNANWELERTNQSGLERCEGEQDKRLHCYASWRNSSGT |
| | (SEQ ID NO: 95) with 6 | IELVKKGCWLDDFNCYDRQECVATEESPQVYFCCCEGNFCNEKFSYFPQME |
| | additional aa at N-terminus | VTQPTSNPVTPKPP |
| 867 | Hybrid hu-ActRIIB-ECD | AETRECIYYNANWELERTNQSGLERCEGEQDKRLHCYASWRNSSGTIELVK |
| | (SEQ ID NO: 96) with 1 | KGCWLDDFNCYDRQECVATEENPEVYFCCCEGNFCNEKFSYFPQMEVTQPT |
| | additional aa at N-terminus | SNPVTPKPP |
| 868 | Hybrid hu-ActRIIB-ECD | EAETRECIYYNANWELERTNQSGLERCEGEQDKRLHCYASWRNSSGTIELV |
| | (SEQ ID NO: 96) with 2 | KKGCWLDDFNCYDRQECVATEENPEVYFCCCEGNFCNEKFSYFPQMEVTQP |
| | additional aa at N-terminus | TSNPVTPKPP |
| 869 | Hybrid hu-ActRIIB-ECD | GEAETRECIYYNANWELERTNQSGLERCEGEQDKRLHCYASWRNSSGTIEL |
| | (SEQ ID NO: 96) with 3 | VKKGCWLDDFNCYDRQECVATEENPEVYFCCCEGNFCNEKFSYFPQMEVT |
| | additional aa at N-terminus | QPTSNPVTPKPP |
| 870 | Hybrid hu-ActRIIB-ECD | RGEAETRECIYYNANWELERTNQSGLERCEGEQDKRLHCYASWRNSSGTIE |
| | (SEQ ID NO: 96) with 4 | LVKKGCWLDDFNCYDRQECVATEENPEVYFCCCEGNFCNEKFSYFPQMEVT |
| | additional aa at N-terminus | QPTSNPVTPKPP |
| 871 | Hybrid hu-ActRIIB-ECD | GRGEAETRECIYYNANWELERTNQSGLERCEGEQDKRLHCYASWRNSSGTI |
| | (SEQ ID NO: 96) with 5 | ELVKKGCWLDDFNCYDRQECVATEENPEVYFCCCEGNFCNEKFSYFPQMEV |
| | additional aa at N-terminus | TQPTSNPVTPKPP |
| 872 | Hybrid hu-ActRIIB-ECD | SGRGEAETRECIYYNANWELERTNQSGLERCEGEQDKRLHCYASWRNSSGT |
| | (SEQ ID NO: 96) with 6 | IELVKKGCWLDDFNCYDRQECVATEENPEVYFCCCEGNFCNEKFSYFPQME |
| | additional aa at N-terminus | VTQPTSNPVTPKPP |
| 873 | Hybrid hu-ActRIIB-ECD | AETRECIYYNANWELERTNQSGLERCEGDKDKRLHCYASWRNSSGTIELVK |
| | (SEQ ID NO: 97) with 1 | KGCWLDDFNCYDRQECVETEENPQVYFCCCEGNFCNEKFSYFPQMEVTQPT |
| | additional aa at N-terminus | SNPVTPKPP |
| 874 | Hybrid hu-ActRIIB-ECD | EAETRECIYYNANWELERTNQSGLERCEGDKDKRLHCYASWRNSSGTIELV |
| | (SEQ ID NO: 97) with 2 | KKGCWLDDFNCYDRQECVETEENPQVYFCCCEGNFCNEKFSYFPQMEVTQP |
| | additional aa at N-terminus | TSNPVTPKPP |
| 875 | Hybrid hu-ActRIIB-ECD | GEAETRECIYYNANWELERTNQSGLERCEGDKDKRLHCYASWRNSSGTIEL |
| | (SEQ ID NO: 97) with 3 | VKKGCWLDDFNCYDRQECVETEENPQVYFCCCEGNFCNEKFSYFPQMEVT |
| | additional aa at N-terminus | QPTSNPVTPKPP |
| 876 | Hybrid hu-ActRIIB-ECD | RGEAETRECIYYNANWELERTNQSGLERCEGDKDKRLHCYASWRNSSGTIE |
| | (SEQ ID NO: 97) with 4 | LVKKGCWLDDFNCYDRQECVETEENPQVYFCCCEGNFCNEKFSYFPQMEVT |
| | additional aa at N-terminus | QPTSNPVTPKPP |
| 877 | Hybrid hu-ActRIIB-ECD | GRGEAETRECIYYNANWELERTNQSGLERCEGDKDKRLHCYASWRNSSGTI |
| | (SEQ ID NO: 97) with 5 | ELVKKGCWLDDFNCYDRQECVETEENPQVYFCCCEGNFCNEKFSYFPQMEV |
| | additional aa at N-terminus | TQPTSNPVTPKPP |
| 878 | Hybrid hu-ActRIIB-ECD | SGRGEAETRECIYYNANWELERTNQSGLERCEGDKDKRLHCYASWRNSSGT |
| | (SEQ ID NO: 97) with 6 | IELVKKGCWLDDFNCYDRQECVETEENPQVYFCCCEGNFCNEKFSYFPQME |
| | additional aa at N-terminus | VTQPTSNPVTPKPP |
| 879 | Hybrid hu-ActRIIB-ECD | AETRECIYYNANWELERTNQSGLERCEGEKDKRLHCYASWRNSSGTIELVK |
| | (SEQ ID NO: 98) with 1 | KGCWLDDFNCYDRQECVAKEENPQVYFCCCEGNFCNEKFSYFPQMEVTQP |
| | additional aa at N-terminus | TSNPVTPKPP |
| 880 | Hybrid hu-ActRIIB-ECD | EAETRECIYYNANWELERTNQSGLERCEGEKDKRLHCYASWRNSSGTIELV |
| | (SEQ ID NO: 98) with 2 | KKGCWLDDFNCYDRQECVAKEENPQVYFCCCEGNFCNEKFSYFPQMEVTQ |
| | additional aa at N-terminus | PTSNPVTPKPP |
| 881 | Hybrid hu-ActRIIB-ECD | GEAETRECIYYNANWELERTNQSGLERCEGEKDKRLHCYASWRNSSGTIEL |
| | (SEQ ID NO: 98) with 3 | VKKGCWLDDFNCYDRQECVAKEENPQVYFCCCEGNFCNEKFSYFPQMEVT |
| | additional aa at N-terminus | QPTSNPVTPKPP |
| 882 | Hybrid hu-ActRIIB-ECD | RGEAETRECIYYNANWELERTNQSGLERCEGEKDKRLHCYASWRNSSGTIE |
| | (SEQ ID NO: 98) with 4 | LVKKGCWLDDFNCYDRQECVAKEENPQVYFCCCEGNFCNEKFSYFPQMEV |
| | additional aa at N-terminus | TQPTSNPVTPKPP |
| 883 | Hybrid hu-ActRIIB-ECD | GRGEAETRECIYYNANWELERTNQSGLERCEGEKDKRLHCYASWRNSSGTI |
| | (SEQ ID NO: 98) with 5 | ELVKKGCWLDDFNCYDRQECVAKEENPQVYFCCCEGNFCNEKFSYFPQME |
| | additional aa at N-terminus | VTQPTSNPVTPKPP |
| 884 | Hybrid hu-ActRIIB-ECD | SGRGEAETRECIYYNANWELERTNQSGLERCEGEKDKRLHCYASWRNSSGT |
| | (SEQ ID NO: 98) with 6 | IELVKKGCWLDDFNCYDRQECVAKEENPQVYFCCCEGNFCNEKFSYFPQME |
| | additional aa at N-terminus | VTQPTSNPVTPKPP |
| 885 | Hybrid hu-ActRIIB-ECD | AETRECIYYNANWELERTNQSGLERCEGDQDKRLHCYASWRNSSGTIELVK |
| | (SEQ ID NO: 99) with 1 | KGCWLDDFNCYDRQECVATKENPQVYFCCCEGNFCNEKFSYFPQMEVTQP |
| | additional aa at N-terminus | TSNPVTPKPP |
| 886 | Hybrid hu-ActRIIB-ECD | EAETRECIYYNANWELERTNQSGLERCEGDQDKRLHCYASWRNSSGTIELV |
| | (SEQ ID NO: 99) with 2 | KKGCWLDDFNCYDRQECVATKENPQVYFCCCEGNFCNEKFSYFPQMEVTQ |
| | additional aa at N-terminus | PTSNPVTPKPP |
| 887 | Hybrid hu-ActRIIB-ECD | GEAETRECIYYNANWELERTNQSGLERCEGDQDKRLHCYASWRNSSGTIEL |
| | (SEQ ID NO: 99) with 3 | VKKGCWLDDFNCYDRQECVATKENPQVYFCCCEGNFCNEKFSYFPQMEVT |
| | additional aa at N-terminus | QPTSNPVTPKPP |
| 888 | Hybrid hu-ActRIIB-ECD | RGEAETRECIYYNANWELERTNQSGLERCEGDQDKRLHCYASWRNSSGTIE |
| | (SEQ ID NO: 99) with 4 | LVKKGCWLDDFNCYDRQECVATKENPQVYFCCCEGNFCNEKFSYFPQMEV |
| | additional aa at N-terminus | TQPTSNPVTPKPP |
| 889 | Hybrid hu-ActRIIB-ECD | GRGEAETRECIYYNANWELERTNQSGLERCEGDQDKRLHCYASWRNSSGTI |
| | (SEQ ID NO: 99) with 5 | ELVKKGCWLDDFNCYDRQECVATKENPQVYFCCCEGNFCNEKFSYFPQME |
| | additional aa at N-terminus | VTQPTSNPVTPKPP |
| 890 | Hybrid hu-ActRIIB-ECD | SGRGEAETRECIYYNANWELERTNQSGLERCEGDQDKRLHCYASWRNSSGT |
| | (SEQ ID NO: 99) with 6 | IELVKKGCWLDDFNCYDRQECVATKENPQVYFCCCEGNFCNEKFSYFPQME |
| | additional aa at N-terminus | VTQPTSNPVTPKPP |
| 891 | Hybrid hu-ActRIIB-ECD | AETRECIYYNANWELERTNQSGLERCEGDKDKRLHCYASWRNSSGTIELVK |
| | (SEQ ID NO: 100) with 1 | QGCWLDDFNCYDRQECVAKKENPQVYFCCCEGNFCNEKFSYFPQMEVTQP |
| | additional aa at N-terminus | TSNPVTPKPP |
| 892 | Hybrid hu-ActRIIB-ECD | EAETRECIYYNANWELERTNQSGLERCEGDKDKRLHCYASWRNSSGTIELV |
| | (SEQ ID NO: 100) with 2 | KQGCWLDDFNCYDRQECVAKKENPQVYFCCCEGNFCNEKFSYFPQMEVTQ |
| | additional aa at N-terminus | PTSNPVTPKPP |
| 893 | Hybrid hu-ActRIIB-ECD | GEAETRECIYYNANWELERTNQSGLERCEGDKDKRLHCYASWRNSSGTIEL |
| | (SEQ ID NO: 100) with 3 | VKQGCWLDDFNCYDRQECVAKKENPQVYFCCCEGNFCNEKFSYFPQMEVT |
| | additional aa at N-terminus | QPTSNPVTPKPP |
| 894 | Hybrid hu-ActRIIB-ECD | RGEAETRECIYYNANWELERTNQSGLERCEGDKDKRLHCYASWRNSSGTIE |
| | (SEQ ID NO: 100) with 4 | LVKQGCWLDDFNCYDRQECVAKKENPQVYFCCCEGNFCNEKFSYFPQMEV |
| | additional aa at N-terminus | TQPTSNPVTPKPP |
| 895 | Hybrid hu-ActRIIB-ECD | GRGEAETRECIYYNANWELERTNQSGLERCEGDKDKRLHCYASWRNSSGTI |
| | (SEQ ID NO: 100) with 5 | ELVKQGCWLDDFNCYDRQECVAKKENPQVYFCCCEGNFCNEKFSYFPQME |
| | additional aa at N-terminus | VTQPTSNPVTPKPP |
| 896 | Hybrid hu-ActRIIB-ECD | SGRGEAETRECIYYNANWELERTNQSGLERCEGDKDKRLHCYASWRNSSGT |
| | (SEQ ID NO: 100) with 6 | IELVKQGCWLDDFNCYDRQECVAKKENPQVYFCCCEGNFCNEKFSYFPQME |
| | additional aa at N-terminus | VTQPTSNPVTPKPP |
| 897 | Hybrid hu-ActRIIB-ECD | AETRECIYYNANWELERTNQSGLERCEGDKDKRLHCYASWRNSSGTIEIVK |
| | (SEQ ID NO: 101) with 1 | QGCWLDDFNCYDRQECVAEKENPQVYFCCCEGNFCNEKFSYFPQMEVTQP |
| | additional aa at N-terminus | TSNPVTPKPP |
| 898 | Hybrid hu-ActRIIB-ECD | EAETRECIYYNANWELERTNQSGLERCEGDKDKRLHCYASWRNSSGTIEIVK |
| | (SEQ ID NO: 101) with 2 | QGCWLDDFNCYDRQECVAEKENPQVYFCCCEGNFCNEKFSYFPQMEVTQP |
| | additional aa at N-terminus | TSNPVTPKPP |
| 899 | Hybrid hu-ActRIIB-ECD | GEAETRECIYYNANWELERTNQSGLERCEGDKDKRLHCYASWRNSSGTIEIV |
| | (SEQ ID NO: 101) with 3 | KQGCWLDDFNCYDRQECVAEKENPQVYFCCCEGNFCNEKFSYFPQMEVTQ |
| | additional aa at N-terminus | PTSNPVTPKPP |
| 900 | Hybrid hu-ActRIIB-ECD | RGEAETRECIYYNANWELERTNQSGLERCEGDKDKRLHCYASWRNSSGTIEI |
| | (SEQ ID NO: 101) with 4 | VKQGCWLDDFNCYDRQECVAEKENPQVYFCCCEGNFCNEKFSYFPQMEVT |
| | additional aa at N-terminus | QPTSNPVTPKPP |
| 901 | Hybrid hu-ActRIIB-ECD | GRGEAETRECIYYNANWELERTNQSGLERCEGDKDKRLHCYASWRNSSGTI |
| | (SEQ ID NO: 101) with 5 | EIVKQGCWLDDFNCYDRQECVAEKENPQVYFCCCEGNFCNEKFSYFPQMEV |
| | additional aa at N-terminus | TQPTSNPVTPKPP |
| 902 | Hybrid hu-ActRIIB-ECD | SGRGEAETRECIYYNANWELERTNQSGLERCEGDKDKRLHCYASWRNSSGT |
| | (SEQ ID NO: 101) with 6 | IEIVKQGCWLDDFNCYDRQECVAEKENPQVYFCCCEGNFCNEKFSYFPQME |
| | additional aa at N-terminus | VTQPTSNPVTPKPP |
| 903 | Hybrid hu-ActRIIB-ECD | AETRECIYYNANWELERTNQSGLERCYGDQDKRLHCYASWRNSSGSIEIVK |
| | (SEQ ID NO: 102) with 1 | QGCWLDDFNCYDRQECVAKKENPQVYFCCCEGNFCNEKFSYFPQMEVTQP |
| | additional aa at N-terminus | TSNPVTPKPP |
| 904 | Hybrid hu-ActRIIB-ECD | EAETRECIYYNANWELERTNQSGLERCYGDQDKRLHCYASWRNSSGSIEIVK |
| | (SEQ ID NO: 102) with 2 | QGCWLDDFNCYDRQECVAKKENPQVYFCCCEGNFCNEKFSYFPQMEVTQP |
| | additional aa at N-terminus | TSNPVTPKPP |
| 905 | Hybrid hu-ActRIIB-ECD | GEAETRECIYYNANWELERTNQSGLERCYGDQDKRLHCYASWRNSSGSIEIV |
| | (SEQ ID NO: 102) with 3 | KQGCWLDDFNCYDRQECVAKKENPQVYFCCCEGNFCNEKFSYFPQMEVTQ |
| | additional aa at N-terminus | PTSNPVTPKPP |
| 906 | Hybrid hu-ActRIIB-ECD | RGEAETRECIYYNANWELERTNQSGLERCYGDQDKRLHCYASWRNSSGSIEI |
| | (SEQ ID NO: 102) with 4 | VKQGCWLDDFNCYDRQECVAKKENPQVYFCCCEGNFCNEKFSYFPQMEVT |
| | additional aa at N-terminus | QPTSNPVTPKPP |
| 907 | Hybrid hu-ActRIIB-ECD | GRGEAETRECIYYNANWELERTNQSGLERCYGDQDKRLHCYASWRNSSGSI |
| | (SEQ ID NO: 102) with 5 | EIVKQGCWLDDFNCYDRQECVAKKENPQVYFCCCEGNFCNEKFSYFPQME |
| | additional aa at N-terminus | VTQPTSNPVTPKPP |
| 908 | Hybrid hu-ActRIIB-ECD | SGRGEAETRECIYYNANWELERTNQSGLERCYGDQDKRLHCYASWRNSSGS |
| | (SEQ ID NO: 102) with 6 | IEIVKQGCWLDDFNCYDRQECVAKKENPQVYFCCCEGNFCNEKFSYFPQME |
| | additional aa at N-terminus | VTQPTSNPVTPKPP |
| 909 | Hybrid hu-ActRIIB-ECD | AETRECIYYNANWELERTNQSGLERCEGEKDKRRHCYASWRNSSGTIEIVKK |
| | (SEQ ID NO: 103) with 1 | GCWLDDFNCYDRQECVATKENPQVYFCCCEGNFCNEKFSYFPQMEVTQPTS |
| | additional aa at N-terminus | NPVTPKPP |
| 910 | Hybrid hu-ActRIIB-ECD | EAETRECIYYNANWELERTNQSGLERCEGEKDKRRHCYASWRNSSGTIEIVK |
| | (SEQ ID NO: 103) with 2 | KGCWLDDFNCYDRQECVATKENPQVYFCCCEGNFCNEKFSYFPQMEVTQP |
| | additional aa at N-terminus | TSNPVTPKPP |
| 911 | Hybrid hu-ActRIIB-ECD | GEAETRECIYYNANWELERTNQSGLERCEGEKDKRRHCYASWRNSSGTIEIV |
| | (SEQ ID NO: 103) with 3 | KKGCWLDDFNCYDRQECVATKENPQVYFCCCEGNFCNEKFSYFPQMEVTQ |
| | additional aa at N-terminus | PTSNPVTPKPP |
| 912 | Hybrid hu-ActRIIB-ECD | RGEAETRECIYYNANWELERTNQSGLERCEGEKDKRRHCYASWRNSSGTIEI |
| | (SEQ ID NO: 103) with 4 | VKKGCWLDDFNCYDRQECVATKENPQVYFCCCEGNFCNEKFSYFPQMEVT |
| | additional aa at N-terminus | QPTSNPVTPKPP |
| 913 | Hybrid hu-ActRIIB-ECD | GRGEAETRECIYYNANWELERTNQSGLERCEGEKDKRRHCYASWRNSSGTI |
| | (SEQ ID NO: 103) with 5 | EIVKKGCWLDDFNCYDRQECVATKENPQVYFCCCEGNFCNEKFSYFPQMEV |
| | additional aa at N-terminus | TQPTSNPVTPKPP |
| 914 | Hybrid hu-ActRIIB-ECD | SGRGEAETRECIYYNANWELERTNQSGLERCEGEKDKRRHCYASWRNSSGT |
| | (SEQ ID NO: 103) with 5 | IEIVKKGCWLDDENCYDRQECVATKENPQVYFCCCEGNFCNEKFSYFPQME |
| | additional aa at N-terminus | VTQPTSNPVTPKPP |
| 915 | Hybrid hu-ActRIIB-ECD | AETRECIYYNANWELERTNQSGLERCYGDQDKRRHCYASWRNSSGTIELVK |
| | (SEQ ID NO: 104) with 1 | KGCWLDDFNCYDRQECVATEENPEVYFCCCEGNFCNEKFSYFPQMEVTQPT |
| | additional aa at N-terminus | SNPVTPKPP |
| 916 | Hybrid hu-ActRIIB-ECD | EAETRECIYYNANWELERINQSGLERCYGDQDKRRHCYASWRNSSGTIELV |
| | (SEQ ID NO: 104) with 2 | KKGCWLDDFNCYDRQECVATEENPEVYFCCCEGNFCNEKFSYFPQMEVTQP |
| | additional aa at N-terminus | TSNPVTPKPP |
| 917 | Hybrid hu-ActRIIB-ECD | GEAETRECIYYNANWELERTNQSGLERCYGDQDKRRHCYASWRNSSGTIEL |
| | (SEQ ID NO: 104) with 3 | VKKGCWLDDFNCYDRQECVATEENPEVYFCCCEGNFCNEKFSYFPQMEVT |
| | additional aa at N-terminus | QPTSNPVTPKPP |
| 918 | Hybrid hu-ActRIIB-ECD | RGEAETRECIYYNANWELERTNQSGLERCYGDQDKRRHCYASWRNSSGTIE |
| | (SEQ ID NO: 104) with 4 | LVKKGCWLDDFNCYDRQECVATEENPEVYFCCCEGNFCNEKFSYFPQMEVT |
| | additional aa at N-terminus | QPTSNPVTPKPP |
| 919 | Hybrid hu-ActRIIB-ECD | GRGEAETRECIYYNANWELERTNQSGLERCYGDQDKRRHCYASWRNSSGTI |
| | (SEQ ID NO: 104) with 5 | ELVKKGCWLDDFNCYDRQECVATEENPEVYFCCCEGNFCNEKFSYFPQMEV |
| | additional aa at N-terminus | TQPTSNPVTPKPP |
| 920 | Hybrid hu-ActRIIB-ECD | SGRGEAETRECIYYNANWELERTNQSGLERCYGDQDKRRHCYASWRNSSG |
| | (SEQ ID NO: 104) with 6 | TIELVKKGCWLDDFNCYDRQECVATEENPEVYFCCCEGNFCNEKFSYFPQM |
| | additional aa at N-terminus | EVTQPTSNPVTPKPP |
| 921 | Hybrid hu-ActRIIB-ECD | AETRECIYYNANWELERTNQSGLERCEGEQDKRRHCYASWRNSSGTIELVK |
| | (SEQ ID NO: 105) with 1 | KGCWLDDFNCYDRQECVATEENPQVYFCCCEGNFCNEKFSYFPQMEVTQPT |
| | additional aa at N-terminus | SNPVTPKPP |
| 922 | Hybrid hu-ActRIIB-ECD | EAETRECIYYNANWELERTNQSGLERCEGEQDKRRHCYASWRNSSGTIELV |
| | (SEQ ID NO: 105) with 2 | KKGCWLDDFNCYDRQECVATEENPQVYFCCCEGNFCNEKFSYFPQMEVTQ |
| | additional aa at N-terminus | PTSNPVTPKPP |
| 923 | Hybrid hu-ActRIIB-ECD | GEAETRECIYYNANWELERTNQSGLERCEGEQDKRRHCYASWRNSSGTIEL |
| | (SEQ ID NO: 105) with 3 | VKKGCWLDDFNCYDRQECVATEENPQVYFCCCEGNFCNEKFSYFPQMEVT |
| | additional aa at N-terminus | QPTSNPVTPKPP |
| 924 | Hybrid hu-ActRIIB-ECD | RGEAETRECIYYNANWELERTNQSGLERCEGEQDKRRHCYASWRNSSGTIE |
| | (SEQ ID NO: 105) with 4 | LVKKGCWLDDFNCYDRQECVATEENPQVYFCCCEGNFCNEKFSYFPQMEV |
| | additional aa at N-terminus | TQPTSNPVTPKPP |
| 925 | Hybrid hu-ActRIIB-ECD | GRGEAETRECIYYNANWELERTNQSGLERCEGEQDKRRHCYASWRNSSGTI |
| | (SEQ ID NO: 105) with 5 | ELVKKGCWLDDFNCYDRQECVATEENPQVYFCCCEGNFCNEKFSYFPQME |
| | additional aa at N-terminus | VTQPTSNPVTPKPP |
| 926 | Hybrid hu-ActRIIB-ECD | SGRGEAETRECIYYNANWELERTNQSGLERCEGEQDKRRHCYASWRNSSGT |
| | (SEQ ID NO: 105) with 6 | IELVKKGCWLDDFNCYDRQECVATEENPQVYFCCCEGNFCNEKFSYFPQME |
| | additional aa at N-terminus | VTQPTSNPVTPKPP |
| 927 | Hybrid hu-ActRIIB-ECD | AETRECIYYNANWELERTNQSGLERCYGEQDKRLHCYASWRNSSGSIEIVKK |
| | (SEQ ID NO: 106) with 1 | GCWLDDFNCYDRTDCVATEENPQVYFCCCEGNFCNEKFSYFPQMEVTQPTS |
| | additional aa at N-terminus | NPVTPKPP |
| 928 | Hybrid hu-ActRIIB-ECD | EAETRECIYYNANWELERTNQSGLERCYGEQDKRLHCYASWRNSSGSIEIVK |
| | (SEQ ID NO: 106) with 2 | KGCWLDDFNCYDRTDCVATEENPQVYFCCCEGNFCNEKFSYFPQMEVTQPT |
| | additional aa at N-terminus | SNPVTPKPP |
| 929 | Hybrid hu-ActRIIB-ECD | GEAETRECIYYNANWELERTNQSGLERCYGEQDKRLHCYASWRNSSGSIEIV |
| | (SEQ ID NO: 106) with 3 | KKGCWLDDFNCYDRTDCVATEENPQVYFCCCEGNFCNEKFSYFPQMEVTQ |
| | additional aa at N-terminus | PTSNPVTPKPP |
| 930 | Hybrid hu-ActRIIB-ECD | RGEAETRECIYYNANWELERTNQSGLERCYGEQDKRLHCYASWRNSSGSIEI |
| | (SEQ ID NO: 106) with 4 | VKKGCWLDDFNCYDRTDCVATEENPQVYFCCCEGNFCNEKFSYFPQMEVT |
| | additional aa at N-terminus | QPTSNPVTPKPP |
| 931 | Hybrid hu-ActRIIB-ECD | GRGEAETRECIYYNANWELERTNQSGLERCYGEQDKRLHCYASWRNSSGSI |
| | (SEQ ID NO: 106) with 5 | EIVKKGCWLDDFNCYDRTDCVATEENPQVYFCCCEGNFCNEKFSYFPQMEV |
| | additional aa at N-terminus | TQPTSNPVTPKPP |
| 932 | Hybrid hu-ActRIIB-ECD | SGRGEAETRECIYYNANWELERTNQSGLERCYGEQDKRLHCYASWRNSSGS |
| | (SEQ ID NO: 106) with 6 | IEIVKKGCWLDDFNCYDRTDCVATEENPQVYFCCCEGNFCNEKFSYFPQME |
| | additional aa at N-terminus | VTQPTSNPVTPKPP |
| 933 | Hybrid hu-ActRIIB-ECD | AETRECIYYNANWELERTNQSGLERCEGEQDKRRHCYASWRNSSGSIELVK |
| | (SEQ ID NO: 107) with 1 | KGCWLDDFNCYDRQECVAKEENPQVYFCCCEGNFCNEKFSYFPQMEVTQP |
| | additional aa at N-terminus | TSNPVTPKPP |
| 934 | Hybrid hu-ActRIIB-ECD | EAETRECIYYNANWELERINQSGLERCEGEQDKRRHCYASWRNSSGSIELV |
| | (SEQ ID NO: 107) with 2 | KKGCWLDDFNCYDRQECVAKEENPQVYFCCCEGNFCNEKFSYFPQMEVTQ |
| | additional aa at N-terminus | PTSNPVTPKPP |
| 935 | Hybrid hu-ActRIIB-ECD | GEAETRECIYYNANWELERTNQSGLERCEGEQDKRRHCYASWRNSSGSIEL |
| | (SEQ ID NO: 107) with 3 | VKKGCWLDDFNCYDRQECVAKEENPQVYFCCCEGNFCNEKFSYFPQMEVT |
| | additional aa at N-terminus | QPTSNPVTPKPP |
| 936 | Hybrid hu-ActRIIB-ECD | RGEAETRECIYYNANWELERTNQSGLERCEGEQDKRRHCYASWRNSSGSIE |
| | (SEQ ID NO: 107) with 4 | LVKKGCWLDDFNCYDRQECVAKEENPQVYFCCCEGNFCNEKFSYFPQMEV |
| | additional aa at N-terminus | TQPTSNPVTPKPP |
| 937 | Hybrid hu-ActRIIB-ECD | GRGEAETRECIYYNANWELERTNQSGLERCEGEQDKRRHCYASWRNSSGSI |
| | (SEQ ID NO: 107) with 5 | ELVKKGCWLDDFNCYDRQECVAKEENPQVYFCCCEGNFCNEKFSYFPQME |
| | additional aa at N-terminus | VTQPTSNPVTPKPP |
| 938 | Hybrid hu-ActRIIB-ECD | SGRGEAETRECIYYNANWELERTNQSGLERCEGEQDKRRHCYASWRNSSGS |
| | (SEQ ID NO: 107) with 6 | IELVKKGCWLDDFNCYDRQECVAKEENPQVYFCCCEGNFCNEKFSYFPQME |
| | additional aa at N-terminus | VTQPTSNPVTPKPP |
| 939 | Hybrid hu-ActRIIB-ECD | AETRECIYYNANWELERTNQSGLERCEGEQDKRRHCYASWRNSSGTIEIVKK |
| | (SEQ ID NO: 108) with 1 | GCWLDDFNCYDRQECVAKEENPQVYFCCCEGNFCNEKFSYFPQMEVTQPTS |
| | additional aa at N-terminus | NPVTPKPP |
| 940 | Hybrid hu-ActRIIB-ECD | EAETRECIYYNANWELERTNQSGLERCEGEQDKRRHCYASWRNSSGTIEIVK |
| | (SEQ ID NO: 108) with 2 | KGCWLDDFNCYDRQECVAKEENPQVYFCCCEGNFCNEKFSYFPQMEVTQP |
| | additional aa at N-terminus | TSNPVTPKPP |
| 941 | Hybrid hu-ActRIIB-ECD | GEAETRECIYYNANWELERTNQSGLERCEGEQDKRRHCYASWRNSSGTIEIV |
| | (SEQ ID NO: 108) with 3 | KKGCWLDDFNCYDRQECVAKEENPQVYFCCCEGNFCNEKFSYFPQMEVTQ |
| | additional aa at N-terminus | PTSNPVTPKPP |
| 942 | Hybrid hu-ActRIIB-ECD | RGEAETRECIYYNANWELERTNQSGLERCEGEQDKRRHCYASWRNSSGTIEI |
| | (SEQ ID NO: 108) with 4 | VKKGCWLDDFNCYDRQECVAKEENPQVYFCCCEGNFCNEKFSYFPQMEVT |
| | additional aa at N-terminus | QPTSNPVTPKPP |
| 943 | Hybrid hu-ActRIIB-ECD | GRGEAETRECIYYNANWELERTNQSGLERCEGEQDKRRHCYASWRNSSGTI |
| | (SEQ ID NO: 108) with 5 | EIVKKGCWLDDFNCYDRQECVAKEENPQVYFCCCEGNFCNEKFSYFPQMEV |
| | additional aa at N-terminus | TQPTSNPVTPKPP |
| 944 | Hybrid hu-ActRIIB-ECD | SGRGEAETRECIYYNANWELERTNQSGLERCEGEQDKRRHCYASWRNSSGT |
| | (SEQ ID NO: 108) with 6 | IEIVKKGCWLDDFNCYDRQECVAKEENPQVYFCCCEGNFCNEKFSYFPQME |
| | additional aa at N-terminus | VTQPTSNPVTPKPP |
| 945 | Hybrid hu-ActRIIB-ECD | AETRECIYYNANWELERTNQSGLERCEGEQDKRRHCYASWRNSSGSIEIVKK |
| | (SEQ ID NO: 109) with 1 | GCWLDDFNCYDRQECVATKENPQVYFCCCEGNFCNEKFSYFPQMEVTQPTS |
| | additional aa at N-terminus | NPVTPKPP |
| 946 | Hybrid hu-ActRIIB-ECD | EAETRECIYYNANWELERTNQSGLERCEGEQDKRRHCYASWRNSSGSIEIVK |
| | (SEQ ID NO: 109) with 2 | KGCWLDDFNCYDRQECVATKENPQVYFCCCEGNFCNEKFSYFPQMEVTQP |
| | additional aa at N-terminus | TSNPVTPKPP |
| 947 | Hybrid hu-ActRIIB-ECD | GEAETRECIYYNANWELERTNQSGLERCEGEQDKRRHCYASWRNSSGSIEIV |
| | (SEQ ID NO: 109) with 3 | KKGCWLDDFNCYDRQECVATKENPQVYFCCCEGNFCNEKFSYFPQMEVTQ |
| | additional aa at N-terminus | PTSNPVTPKPP |
| 948 | Hybrid hu-ActRIIB-ECD | RGEAETRECIYYNANWELERTNQSGLERCEGEQDKRRHCYASWRNSSGSIEI |
| | (SEQ ID NO: 109) with 4 | VKKGCWLDDFNCYDRQECVATKENPQVYFCCCEGNFCNEKFSYFPQMEVT |
| | additional aa at N-terminus | QPTSNPVTPKPP |
| 949 | Hybrid hu-ActRIIB-ECD | GRGEAETRECIYYNANWELERTNQSGLERCEGEQDKRRHCYASWRNSSGSI |
| | (SEQ ID NO: 109) with 5 | EIVKKGCWLDDFNCYDRQECVATKENPQVYFCCCEGNFCNEKFSYFPQMEV |
| | additional aa at N-terminus | TQPTSNPVTPKPP |
| 950 | Hybrid hu-ActRIIB-ECD | SGRGEAETRECIYYNANWELERTNQSGLERCEGEQDKRRHCYASWRNSSGS |
| | (SEQ ID NO: 109) with 6 | IEIVKKGCWLDDFNCYDRQECVATKENPQVYFCCCEGNFCNEKFSYFPQME |
| | additional aa at N-terminus | VTQPTSNPVTPKPP |
| 951 | Hybrid hu-ActRIIB-ECD | AETRECIYYNANWELERTNQSGLERCEGDQDKRLHCYASWRNSSGTIEIVK |
| | (SEQ ID NO: 110) with 1 | KGCWLDDFNCYDRQECVATKENPQVYFCCCEGNFCNEKFSYFPQMEVTQP |
| | additional aa at N-terminus | TSNPVTPKPP |
| 952 | Hybrid hu-ActRIIB-ECD | EAETRECIYYNANWELERTNQSGLERCEGDQDKRLHCYASWRNSSGTIEIVK |
| | (SEQ ID NO: 110) with 2 | KGCWLDDFNCYDRQECVATKENPQVYFCCCEGNFCNEKFSYFPQMEVTQP |
| | additional aa at N-terminus | TSNPVTPKPP |
| 953 | Hybrid hu-ActRIIB-ECD | GEAETRECIYYNANWELERTNQSGLERCEGDQDKRLHCYASWRNSSGTIEIV |
| | (SEQ ID NO: 110) with 3 | KKGCWLDDFNCYDRQECVATKENPQVYFCCCEGNFCNEKFSYFPQMEVTQ |
| | additional aa at N-terminus | PTSNPVTPKPP |
| 954 | Hybrid hu-ActRIIB-ECD | RGEAETRECIYYNANWELERTNQSGLERCEGDQDKRLHCYASWRNSSGTIEI |
| | (SEQ ID NO: 110) with 4 | VKKGCWLDDFNCYDRQECVATKENPQVYFCCCEGNFCNEKFSYFPQMEVT |
| | additional aa at N-terminus | QPTSNPVTPKPP |
| 955 | Hybrid hu-ActRIIB-ECD | GRGEAETRECIYYNANWELERTNQSGLERCEGDQDKRLHCYASWRNSSGTI |
| | (SEQ ID NO: 110) with 5 | EIVKKGCWLDDFNCYDRQECVATKENPQVYFCCCEGNFCNEKFSYFPQMEV |
| | additional aa at N-terminus | TQPTSNPVTPKPP |
| 956 | Hybrid hu-ActRIIB-ECD | SGRGEAETRECIYYNANWELERTNQSGLERCEGDQDKRLHCYASWRNSSGT |
| | (SEQ ID NO: 110) with 6 | IEIVKKGCWLDDFNCYDRQECVATKENPQVYFCCCEGNFCNEKFSYFPQME |
| | additional aa at N-terminus | VTQPTSNPVTPKPP |
| 957 | Hybrid hu-ActRIIB-ECD | AETRECIYYNANWELERTNQSGLERCEGDQDKRLHCYASWRNSSGSIELVK |
| | (SEQ ID NO: 111) with 1 | KGCWLDDFNCYDRQECVATKENPQVYFCCCEGNFCNEKFSYFPQMEVTQP |
| | additional aa at N-terminus | TSNPVTPKPP |
| 958 | Hybrid hu-ActRIIB-ECD | EAETRECIYYNANWELERTNQSGLERCEGDQDKRLHCYASWRNSSGSIELV |
| | (SEQ ID NO: 111) with 2 | KKGCWLDDFNCYDRQECVATKENPQVYFCCCEGNFCNEKFSYFPQMEVTQ |
| | additional aa at N-terminus | PTSNPVTPKPP |
| 959 | Hybrid hu-ActRIIB-ECD | GEAETRECIYYNANWELERTNQSGLERCEGDQDKRLHCYASWRNSSGSIEL |
| | (SEQ ID NO: 111) with 3 | VKKGCWLDDFNCYDRQECVATKENPQVYFCCCEGNFCNEKFSYFPQMEVT |
| | additional aa at N-terminus | QPTSNPVTPKPP |
| 960 | Hybrid hu-ActRIIB-ECD | RGEAETRECIYYNANWELERTNQSGLERCEGDQDKRLHCYASWRNSSGSIE |
| | (SEQ ID NO: 111) with 4 | LVKKGCWLDDFNCYDRQECVATKENPQVYFCCCEGNFCNEKFSYFPQMEV |
| | additional aa at N-terminus | TQPTSNPVTPKPP |
| 961 | Hybrid hu-ActRIIB-ECD | GRGEAETRECIYYNANWELERTNQSGLERCEGDQDKRLHCYASWRNSSGSI |
| | (SEQ ID NO: 111) with 5 | ELVKKGCWLDDFNCYDRQECVATKENPQVYFCCCEGNFCNEKFSYFPQME |
| | additional aa at N-terminus | VTQPTSNPVTPKPP |
| 962 | Hybrid hu-ActRIIB-ECD | SGRGEAETRECIYYNANWELERTNQSGLERCEGDQDKRLHCYASWRNSSGS |
| | (SEQ ID NO: 111) with 6 | IELVKKGCWLDDFNCYDRQECVATKENPQVYFCCCEGNFCNEKFSYFPQME |
| | additional aa at N-terminus | VTQPTSNPVTPKPP |
| 963 | Hybrid hu-ActRIIB-ECD | AETRECIYYNANWELERTNQSGLERCEGDQDKRLHCYASWRNSSGTIELVK |
| | (SEQ ID NO: 112) with 1 | KGCWLDDFNCYDRQECVATKENPQVYFCCCEGNFCNEKFSYFPQMEVTQP |
| | additional aa at N-terminus | TSNPVTPKPP |
| 964 | Hybrid hu-ActRIIB-ECD | EAETRECIYYNANWELERTNQSGLERCEGDQDKRLHCYASWRNSSGTIELV |
| | (SEQ ID NO: 112) with 2 | KKGCWLDDFNCYDRQECVATKENPQVYFCCCEGNFCNEKFSYFPQMEVTQ |
| | additional aa at N-terminus | PTSNPVTPKPP |
| 965 | Hybrid hu-ActRIIB-ECD | GEAETRECIYYNANWELERTNQSGLERCEGDQDKRLHCYASWRNSSGTIEL |
| | (SEQ ID NO: 112) with 3 | VKKGCWLDDFNCYDRQECVATKENPQVYFCCCEGNFCNEKFSYFPQMEVT |
| | additional aa at N-terminus | QPTSNPVTPKPP |
| 966 | Hybrid hu-ActRIIB-ECD | RGEAETRECIYYNANWELERTNQSGLERCEGDQDKRLHCYASWRNSSGTIE |
| | (SEQ ID NO: 112) with 4 | LVKKGCWLDDFNCYDRQECVATKENPQVYFCCCEGNFCNEKFSYFPQMEV |
| | additional aa at N-terminus | TQPTSNPVTPKPP |
| 967 | Hybrid hu-ActRIIB-ECD | GRGEAETRECIYYNANWELERTNQSGLERCEGDQDKRLHCYASWRNSSGTI |
| | (SEQ ID NO: 112) with 5 | ELVKKGCWLDDFNCYDRQECVATKENPQVYFCCCEGNFCNEKFSYFPQME |
| | additional aa at N-terminus | VTQPTSNPVTPKPP |
| 968 | Hybrid hu-ActRIIB-ECD | SGRGEAETRECIYYNANWELERTNQSGLERCEGDQDKRLHCYASWRNSSGT |
| | (SEQ ID NO: 112) with 6 | IELVKKGCWLDDFNCYDRQECVATKENPQVYFCCCEGNFCNEKFSYFPQME |
| | additional aa at N-terminus | VTQPTSNPVTPKPP |
| 969 | Hybrid hu-ActRIIB-ECD | AETRECIYYNANWELERTNQSGLERCEGEQDKRLHCYASWRNSSGTIEIVKK |
| | (SEQ ID NO: 113) with 1 | GCWLDDFNCYDRQECVATKENPQVYFCCCEGNFCNEKFSYFPQMEVTQPTS |
| | additional aa at N-terminus | NPVTPKPP |
| 970 | Hybrid hu-ActRIIB-ECD | EAETRECIYYNANWELERTNQSGLERCEGEQDKRLHCYASWRNSSGTIEIVK |
| | (SEQ ID NO: 113) with 2 | KGCWLDDFNCYDRQECVATKENPQVYFCCCEGNFCNEKFSYFPQMEVTQP |
| | additional aa at N-terminus | TSNPVTPKPP |
| 971 | Hybrid hu-ActRIIB-ECD | GEAETRECIYYNANWELERTNQSGLERCEGEQDKRLHCYASWRNSSGTIEIV |
| | (SEQ ID NO: 113) with 3 | KKGCWLDDFNCYDRQECVATKENPQVYFCCCEGNFCNEKFSYFPQMEVTQ |
| | additional aa at N-terminus | PTSNPVTPKPP |
| 972 | Hybrid hu-ActRIIB-ECD | RGEAETRECIYYNANWELERTNQSGLERCEGEQDKRLHCYASWRNSSGTIEI |
| | (SEQ ID NO: 113) with 4 | VKKGCWLDDFNCYDRQECVATKENPQVYFCCCEGNFCNEKFSYFPQMEVT |
| | additional aa at N-terminus | QPTSNPVTPKPP |
| 973 | Hybrid hu-ActRIIB-ECD | GRGEAETRECIYYNANWELERTNQSGLERCEGEQDKRLHCYASWRNSSGTI |
| | (SEQ ID NO: 113) with 5 | EIVKKGCWLDDFNCYDRQECVATKENPQVYFCCCEGNFCNEKFSYFPQMEV |
| | additional aa at N-terminus | TQPTSNPVTPKPP |
| 974 | Hybrid hu-ActRIIB-ECD | SGRGEAETRECIYYNANWELERTNQSGLERCEGEQDKRLHCYASWRNSSGT |
| | (SEQ ID NO: 113) with 6 | IEIVKKGCWLDDFNCYDRQECVATKENPQVYFCCCEGNFCNEKFSYFPQME |
| | additional aa at N-terminus | VTQPTSNPVTPKPP |
| 975 | Hybrid hu-ActRIIB-ECD | AETRECIYYNANWELERTNQSGLERCEGEQDKRLHCYASWRNSSGTIELVK |
| | (SEQ ID NO: 114) with 1 | KGCWLDDFNCYDRQECVATKENPQVYFCCCEGNFCNEKFSYFPQMEVTQP |
| | additional aa at N-terminus | TSNPVTPKPP |
| 976 | Hybrid hu-ActRIIB-ECD | EAETRECIYYNANWELERTNQSGLERCEGEQDKRLHCYASWRNSSGTIELV |
| | (SEQ ID NO: 114) with 2 | KKGCWLDDFNCYDRQECVATKENPQVYFCCCEGNFCNEKFSYFPQMEVTQ |
| | additional aa at N-terminus | PTSNPVTPKPP |
| 977 | Hybrid hu-ActRIIB-ECD | GEAETRECIYYNANWELERTNQSGLERCEGEQDKRLHCYASWRNSSGTIEL |
| | (SEQ ID NO: 114) with 3 | VKKGCWLDDFNCYDRQECVATKENPQVYFCCCEGNFCNEKFSYFPQMEVT |
| | additional aa at N-terminus | QPTSNPVTPKPP |
| 978 | Hybrid hu-ActRIIB-ECD | RGEAETRECIYYNANWELERTNQSGLERCEGEQDKRLHCYASWRNSSGTIE |
| | (SEQ ID NO: 114) with 4 | LVKKGCWLDDFNCYDRQECVATKENPQVYFCCCEGNFCNEKFSYFPQMEV |
| | additional aa at N-terminus | TQPTSNPVTPKPP |
| 979 | Hybrid hu-ActRIIB-ECD | GRGEAETRECIYYNANWELERTNQSGLERCEGEQDKRLHCYASWRNSSGTI |
| | (SEQ ID NO: 114) with 5 | ELVKKGCWLDDFNCYDRQECVATKENPQVYFCCCEGNFCNEKFSYFPQME |
| | additional aa at N-terminus | VTQPTSNPVTPKPP |
| 980 | Hybrid hu-ActRIIB-ECD | SGRGEAETRECIYYNANWELERTNQSGLERCEGEQDKRLHCYASWRNSSGT |
| | (SEQ ID NO: 114) with 6 | IELVKKGCWLDDFNCYDRQECVATKENPQVYFCCCEGNFCNEKFSYFPQME |
| | additional aa at N-terminus | VTQPTSNPVTPKPP |
| 981 | Hybrid hu-ActRIIB-ECD | AETRECIYYNANWELERTNQSGLERCEGDQDKRLHCYASWRNSSGTIEIVK |
| | (SEQ ID NO: 115) with 1 | KGCWLDDFNCYDRQECVAKKENPQVYFCCCEGNFCNEKFSYFPQMEVTQP |
| | additional aa at N-terminus | TSNPVTPKPP |
| 982 | Hybrid hu-ActRIIB-ECD | EAETRECIYYNANWELERTNQSGLERCEGDQDKRLHCYASWRNSSGTIEIVK |
| | (SEQ ID NO: 115) with 2 | KGCWLDDFNCYDRQECVAKKENPQVYFCCCEGNFCNEKFSYFPQMEVTQP |
| | additional aa at N-terminus | TSNPVTPKPP |
| 983 | Hybrid hu-ActRIIB-ECD | GEAETRECIYYNANWELERTNQSGLERCEGDQDKRLHCYASWRNSSGTIEIV |
| | (SEQ ID NO: 115) with 3 | KKGCWLDDFNCYDRQECVAKKENPQVYFCCCEGNFCNEKFSYFPQMEVTQ |
| | additional aa at N-terminus | PTSNPVTPKPP |
| 984 | Hybrid hu-ActRIIB-ECD | RGEAETRECIYYNANWELERTNQSGLERCEGDQDKRLHCYASWRNSSGTIEI |
| | (SEQ ID NO: 115) with 4 | VKKGCWLDDFNCYDRQECVAKKENPQVYFCCCEGNFCNEKFSYFPQMEVT |
| | additional aa at N-terminus | QPTSNPVTPKPP |
| 985 | Hybrid hu-ActRIIB-ECD | GRGEAETRECIYYNANWELERTNQSGLERCEGDQDKRLHCYASWRNSSGTI |
| | (SEQ ID NO: 115) with 5 | EIVKKGCWLDDFNCYDRQECVAKKENPQVYFCCCEGNFCNEKFSYFPQME |
| | additional aa at N-terminus | VTQPTSNPVTPKPP |
| 986 | Hybrid hu-ActRIIB-ECD | SGRGEAETRECIYYNANWELERTNQSGLERCEGDQDKRLHCYASWRNSSGT |
| | (SEQ ID NO: 115) with 6 | IEIVKKGCWLDDFNCYDRQECVAKKENPQVYFCCCEGNFCNEKFSYFPQME |
| | additional aa at N-terminus | VTQPTSNPVTPKPP |
| 987 | Hybrid hu-ActRIIB-ECD | AETQECLFFNANWEKDRTNQTGVEPCYGDKDKRRHCFATWKNISGSIEIVK |
| | (SEQ ID NO: 116) with 1 | QGCWLDDINCYDRTDCVEKKDSPEVYFCCCEGNMCNERFTHLPEAGGPEVT |
| | additional aa at N-terminus | YEPPPTAPT |
| 988 | Hybrid hu-ActRIIB-ECD | EAETQECLFFNANWEKDRTNQTGVEPCYGDKDKRRHCFATWKNISGSIEIV |
| | (SEQ ID NO: 116) with 2 | KQGCWLDDINCYDRTDCVEKKDSPEVYFCCCEGNMCNERFTHLPEAGGPEV |
| | additional aa at N-terminus | TYEPPPTAPT |
| 989 | Hybrid hu-ActRIIB-ECD | GEAETQECLFFNANWEKDRTNQTGVEPCYGDKDKRRHCFATWKNISGSIEI |
| | (SEQ ID NO: 116) with 3 | VKQGCWLDDINCYDRTDCVEKKDSPEVYFCCCEGNMCNERFTHLPEAGGPE |
| | additional aa at N-terminus | VTYEPPPTAPT |
| 990 | Hybrid hu-ActRIIB-ECD | RGEAETQECLFFNANWEKDRTNQTGVEPCYGDKDKRRHCFATWKNISGSIEI |
| | (SEQ ID NO: 116) with 4 | VKQGCWLDDINCYDRTDCVEKKDSPEVYFCCCEGNMCNERFTHLPEAGGPE |
| | additional aa at N-terminus | VTYEPPPTAPT |
| 991 | Hybrid hu-ActRIIB-ECD | GRGEAETQECLFFNANWEKDRTNQTGVEPCYGDKDKRRHCFATWKNISGSI |
| | (SEQ ID NO: 116) with 5 | EIVKQGCWLDDINCYDRTDCVEKKDSPEVYFCCCEGNMCNERFTHLPEAGG |
| | additional aa at N-terminus | PEVTYEPPPTAPT |
| 992 | Hybrid hu-ActRIIB-ECD | SGRGEAETQECLFFNANWEKDRTNQTGVEPCYGDKDKRRHCFATWKNISGS |
| | (SEQ ID NO: 116) with 6 | IEIVKQGCWLDDINCYDRTDCVEKKDSPEVYFCCCEGNMCNERFTHLPEAG |
| | additional aa at N-terminus | GPEVTYEPPPTAPT |
| 993 | Hybrid hu-ActRIIB-ECD | AETRECIYYNANWELERTNQSGLERCYGDKDKRRHCYASWRNSSGTIEL VK |
| | (SEQ ID NO: 117) with 1 | KGCWLDDINCYDRQECVATKENPQVYFCCCEGNFCNERFTHLPEAGGPEVT |
| | additional aa at N-terminus | YEPPPTAPT |
| 994 | Hybrid hu-ActRIIB-ECD | EAETRECIYYNANWELERTNQSGLERCYGDKDKRRHCYASWRNSSGTIELV |
| | (SEQ ID NO: 117) with 2 | KKGCWLDDINCYDRQECVATKENPQVYFCCCEGNFCNERFTHLPEAGGPEV |
| | additional aa at N-terminus | TYEPPPTAPT |
| 995 | Hybrid hu-ActRIIB-ECD | GEAETRECIYYNANWELERTNQSGLERCYGDKDKRRHCYASWRNSSGTIEL |
| | (SEQ ID NO: 117) with 3 | VKKGCWLDDINCYDRQECVATKENPQVYFCCCEGNFCNERFTHLPEAGGPE |
| | additional aa at N-terminus | VTYEPPPTAPT |
| 996 | Hybrid hu-ActRIIB-ECD | RGEAETRECIYYNANWELERTNQSGLERCYGDKDKRRHCYASWRNSSGTIE |
| | (SEQ ID NO: 117) with 4 | LVKKGCWLDDINCYDRQECVATKENPQVYFCCCEGNFCNERFTHLPEAGGP |
| | additional aa at N-terminus | EVTYEPPPTAPT |
| 997 | Hybrid hu-ActRIIB-ECD | GRGEAETRECIYYNANWELERTNQSGLERCYGDKDKRRHCYASWRNSSGTI |
| | (SEQ ID NO: 117) with 5 | ELVKKGCWLDDINCYDRQECVATKENPQVYFCCCEGNFCNERFTHLPEAGG |
| | additional aa at N-terminus | PEVTYEPPPTAPT |
| 998 | Hybrid hu-ActRIIB-ECD | SGRGEAETRECIYYNANWELERTNQSGLERCYGDKDKRRHCYASWRNSSG |
| | (SEQ ID NO: 117) with 6 | TIELVKKGCWLDDINCYDRQECVATKENPQVYFCCCEGNFCNERFTHLPEA |
| | additional aa at N-terminus | GGPEVTYEPPPTAPT |
| 1000 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDINCYDRTDCVEKKDSPEVYFCCCEGNFCNERFTHLPEAGGPEVT |
| | 3) with 1 additional aa at N- | YEPPPTAPT |
| | terminus | |
| 1001 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDINCYDRTDCVEKKDSPEVYFCCCEGNFCNERFTHLPEAGGPEV |
| | 3) with 2 additional aa at N- | TYEPPPTAPT |
| | terminus | |
| 1002 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDINCYDRTDCVEKKDSPEVYFCCCEGNFCNERFTHLPEAGGPE |
| | 3) with 3 additional aa at N- | VTYEPPPTAPT |
| | terminus | |
| 1003 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDINCYDRTDCVEKKDSPEVYFCCCEGNFCNERFTHLPEAGGP |
| | 3) with 4 additional aa at N- | EVTYEPPPTAPT |
| | terminus | |
| 1004 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDINCYDRTDCVEKKDSPEVYFCCCEGNFCNERFTHLPEAGG |
| | 3) with 5 additional aa at N- | PEVTYEPPPTAPT |
| | terminus | |
| 1005 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDINCYDRTDCVEKKDSPEVYFCCCEGNFCNERFTHLPEAG |
| | 3) with 6 additional aa at N- | GPEVTYEPPPTAPT |
| | terminus | |
| 1006 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDFNCYDRTDCVEKKDSPEVYFCCCEGNFCNERFTHLPEAGGPEVT |
| | 4) with 1 additional aa at N- | YEPPPTAPT |
| | terminus | |
| 1007 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDFNCYDRTDCVEKKDSPEVYFCCCEGNFCNERFTHLPEAGGPEV |
| | 4) with 2 additional aa at N- | TYEPPPTAPT |
| | terminus | |
| 1008 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDFNCYDRTDCVEKKDSPEVYFCCCEGNFCNERFTHLPEAGGPE |
| | 4) with 3 additional aa at N- | VTYEPPPTAPT |
| | terminus | |
| 1009 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDFNCYDRTDCVEKKDSPEVYFCCCEGNFCNERFTHLPEAGGP |
| | 4) with 4 additional aa at N- | EVTYEPPPTAPT |
| | terminus | |
| 1010 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDFNCYDRTDCVEKKDSPEVYFCCCEGNFCNERFTHLPEAGG |
| | 4) with 5 additional aa at N- | PEVTYEPPPTAPT |
| | terminus | |
| 1011 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDFNCYDRTDCVEKKDSPEVYFCCCEGNFCNERFTHLPEA |
| | 4) with 6 additional aa at N- | GGPEVTYEPPPTAPT |
| | terminus | |
| 1012 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDINCYDRTDCVEKKDSPEVYFCCCEGNFCNERFTHLPEAGGPEVT |
| | 5) with 1 additional aa at N- | YEPPPTAPT |
| | terminus | |
| 1013 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDINCYDRTDCVEKKDSPEVYFCCCEGNFCNERFTHLPEAGGPEV |
| | 5) with 2 additional aa at N- | TYEPPPTAPT |
| | terminus | |
| 1014 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDINCYDRTDCVEKKDSPEVYFCCCEGNFCNERFTHLPEAGGPE |
| | 5) with 3 additional aa at N- | VTYEPPPTAPT |
| | terminus | |
| 1015 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDINCYDRTDCVEKKDSPEVYFCCCEGNFCNERFTHLPEAGGP |
| | 5) with 4 additional aa at N- | EVTYEPPPTAPT |
| | terminus | |
| 1016 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDINCYDRTDCVEKKDSPEVYFCCCEGNFCNERFTHLPEAGG |
| | 5) with 5 additional aa at N- | PEVTYEPPPTAPT |
| | terminus | |
| 1017 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDINCYDRTDCVEKKDSPEVYFCCCEGNFCNERFTHLPEAG |
| | 5) with 6 additional aa at N- | GPEVTYEPPPTAPT |
| | terminus | |
| 1018 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDINCYDRTDCVEKKDSPQVYFCCCEGNFCNERFTHLPEAGGPEVT |
| | 6) with 1 additional aa at N- | YEPPPTAPT |
| | terminus | |
| 1019 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDINCYDRTDCVEKKDSPQVYFCCCEGNFCNERFTHLPEAGGPEV |
| | 6) with 2 additional aa at N- | TYEPPPTAPT |
| | terminus | |
| 1020 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDINCYDRTDCVEKKDSPQVYFCCCEGNFCNERFTHLPEAGGPE |
| | 6) with 3 additional aa at N- | VTYEPPPTAPT |
| | terminus | |
| 1021 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDINCYDRTDCVEKKDSPQVYFCCCEGNFCNERFTHLPEAGGP |
| | 6) with 4 additional aa at N- | EVTYEPPPTAPT |
| | terminus | |
| 1022 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDINCYDRTDCVEKKDSPQVYFCCCEGNFCNERFTHLPEAGG |
| | 6) with 5 additional aa at N- | PEVTYEPPPTAPT |
| | terminus | |
| 1023 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDINCYDRTDCVEKKDSPQVYFCCCEGNFCNERFTHLPEAG |
| | 6) with 6 additional aa at N- | GPEVTYEPPPTAPT |
| | terminus | |
| 1024 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRTDCVEKKDSPQVYFCCCEGNFCNERFTHLPEAGGPEVT |
| | 7) with 1 additional aa at N- | YEPPPTAPT |
| | terminus | |
| 1025 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRTDCVEKKDSPQVYFCCCEGNFCNERFTHLPEAGGPEV |
| | 7) with 2 additional aa at N- | TYEPPPTAPT |
| | terminus | |
| 1026 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRTDCVEKKDSPQVYFCCCEGNFCNERFTHLPEAGGPE |
| | 7) with 3 additional aa at N- | VTYEPPPTAPT |
| | terminus | |
| 1027 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRTDCVEKKDSPQVYFCCCEGNFCNERFTHLPEAGGP |
| | 7) with 4 additional aa at N- | EVTYEPPPTAPT |
| | terminus | |
| 1028 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRTDCVEKKDSPQVYFCCCEGNFCNERFTHLPEAG |
| | 7) with 5 additional aa at N- | GPEVTYEPPPTAPT |
| | terminus | |
| 1029 | N24Q mutant of Hybrid hu- | SGRGEA |
| | ActRIIB-ECD (SEQ ID NO: | ETRECIYYNANWELERTQQSGLERCEGEQDKRLHCYASWRNSSGTIELVKK |
| | 7) with 6 additional aa at N- | GCWLDDFNCYDRTDCVEKKDSPQVYFCCCEGNFCNERFTHLPEAGGPEVTY |
| | terminus | EPPPTAPT |
| 1030 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRTDCVEKKDSPEVYFCCCEGNFCNERFTHLPEAGGPEVT |
| | 8) with 1 additional aa at N- | YEPPPTAPT |
| | terminus | |
| 1031 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRTDCVEKKDSPEVYFCCCEGNFCNERFTHLPEAGGPEV |
| | 8) with 2 additional aa at N- | TYEPPPTAPT |
| | terminus | |
| 1032 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRTDCVEKKDSPEVYFCCCEGNFCNERFTHLPEAGGPE |
| | 8) with 3 additional aa at N- | VTYEPPPTAPT |
| | terminus | |
| 1033 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRTDCVEKKDSPEVYFCCCEGNFCNERFTHLPEAGGP |
| | 8) with 4 additional aa at N- | EVTYEPPPTAPT |
| | terminus | |
| 1034 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRTDCVEKKDSPEVYFCCCEGNFCNERFTHLPEAGG |
| | 8) with 5 additional aa at N- | PEVTYEPPPTAPT |
| | terminus | |
| 1035 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRTDCVEKKDSPEVYFCCCEGNFCNERFTHLPEAG |
| | 8) with 6 additional aa at N- | GPEVTYEPPPTAPT |
| | terminus | |
| 1036 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVEKKDSPEVYFCCCEGNFCNERFTHLPEAGGPEVT |
| | 9) with 1 additional aa at N- | YEPPPTAPT |
| | terminus | |
| 1037 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVEKKDSPEVYFCCCEGNFCNERFTHLPEAGGPEV |
| | 9) with 2 additional aa at N- | TYEPPPTAPT |
| | terminus | |
| 1038 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVEKKDSPEVYFCCCEGNFCNERFTHLPEAGGPE |
| | 9) with 3 additional aa at N- | VTYEPPPTAPT |
| | terminus | |
| 1039 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVEKKDSPEVYFCCCEGNFCNERFTHLPEAGGP |
| | 9) with 4 additional aa at N- | EVTYEPPPTAPT |
| | terminus | |
| 1040 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVEKKDSPEVYFCCCEGNFCNERFTHLPEAGG |
| | 9) with 5 additional aa at N- | PEVTYEPPPTAPT |
| | terminus | |
| 1041 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVEKKDSPEVYFCCCEGNFCNERFTHLPEAG |
| | 9) with 6 additional aa at N- | GPEVTYEPPPTAPT |
| | terminus | |
| 1042 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVEKKDSPQVYFCCCEGNFCNERFTHLPEAGGPEVT |
| | 10) with 1 additional aa at N- | YEPPPTAPT |
| | terminus | |
| 1043 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVEKKDSPQVYFCCCEGNFCNERFTHLPEAGGPEV |
| | 10) with 2 additional aa at N- | TYEPPPTAPT |
| | terminus | |
| 1044 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVEKKDSPQVYFCCCEGNFCNERFTHLPEAGGPE |
| | 10) with 3 additional aa at N- | VTYEPPPTAPT |
| | terminus | |
| 1045 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVEKKDSPQVYFCCCEGNFCNERFTHLPEAGGP |
| | 10) with 4 additional aa at N- | EVTYEPPPTAPT |
| | terminus | |
| 1046 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVEKKDSPQVYFCCCEGNFCNERFTHLPEAG |
| | 10) with 5 additional aa at N- | GPEVTYEPPPTAPT |
| | terminus | |
| 1047 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVEKKDSPQVYFCCCEGNFCNERFTHLPEAG |
| | 10) with 6 additional aa at N- | GPEVTYEPPPTAPT |
| | terminus | |
| 1048 | N19Q mutant of Hybrid hu- | ATRECIYYNANWELERTQQSGLERCEGEQDKRLHCYASWRNSSGTIELVKK |
| | ActRIIB-ECD (SEQ ID NO: | GCWLDDINCYDRQECVEKKDSPEVYFCCCEGNFCNERFTHLPEAGGPEVTY |
| | 11) with 1 additional aa at N- | EPPPTAPT |
| | terminus | |
| 1049 | N20Q mutant of Hybrid hu- | EATRECIYYNANWELERTQQSGLERCEGEQDKRLHCYASWRNSSGTIELVK |
| | ActRIIB-ECD (SEQ ID NO: | KGCWLDDINCYDRQECVEKKDSPEVYFCCCEGNFCNERFTHLPEAGGPEVT |
| | 11) with 2 additional aa at N- | YEPPPTAPT |
| | terminus | |
| 1050 | N21Q mutant of Hybrid hu- | GEATRECIYYNANWELERTQQSGLERCEGEQDKRLHCYASWRNSSGTIELV |
| | ActRIIB-ECD (SEQ ID NO: | KKGCWLDDINCYDRQECVEKKDSPEVYFCCCEGNFCNERFTHLPEAGGPEV |
| | 11) with 3 additional aa at N- | TYEPPPTAPT |
| | terminus | |
| 1051 | N22Q mutant of Hybrid hu- | RGEATRECIYYNANWELERTQQSGLERCEGEQDKRLHCYASWRNSSGTIEL |
| | ActRIIB-ECD (SEQ ID NO: | VKKGCWLDDINCYDRQECVEKKDSPEVYFCCCEGNFCNERFTHLPEAGGPE |
| | 11) with 4 additional aa at N- | VTYEPPPTAPT |
| | terminus | |
| 1052 | N23Q mutant of Hybrid hu- | GRGEATRECIYYNANWELERTQQSGLERCEGEQDKRLHCYASWRNSSGTIE |
| | ActRIIB-ECD (SEQ ID NO: | LVKKGCWLDDINCYDRQECVEKKDSPEVYFCCCEGNFCNERFTHLPEAGGP |
| | 11) with 5 additional aa at N- | EVTYEPPPTAPT |
| | terminus | |
| 1053 | N24Q mutant of Hybrid hu- | SGRGEATRECIYYNANWELERTQQSGLERCEGEQDKRLHCYASWRNSSGTI |
| | ActRIIB-ECD (SEQ ID NO: | ELVKKGCWLDDINCYDRQECVEKKDSPEVYFCCCEGNFCNERFTHLPEAGG |
| | 11) with 6 additional aa at N- | PEVTYEPPPTAPT |
| | terminus | |
| 1054 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRTDCVEKKDSPEVYFCCCEGNMCNERFTHLPEAGGPEV |
| | 12) with 1 additional aa at N- | TYEPPPTAPT |
| | terminus | |
| 1055 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRTDCVEKKDSPEVYFCCCEGNMCNERFTHLPEAGGPE |
| | 12) with 2 additional aa at N- | VTYEPPPTAPT |
| | terminus | |
| 1056 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRTDCVEKKDSPEVYFCCCEGNMCNERFTHLPEAGGP |
| | 12) with 3 additional aa at N- | EVTYEPPPTAPT |
| | terminus | |
| 1057 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRTDCVEKKDSPEVYFCCCEGNMCNERFTHLPEAGG |
| | 12) with 4 additional aa at N- | PEVTYEPPPTAPT |
| | terminus | |
| 1058 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRTDCVEKKDSPEVYFCCCEGNMCNERFTHLPEAG |
| | 12) with 5 additional aa at N- | GPEVTYEPPPTAPT |
| | terminus | |
| 1059 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRTDCVEKKDSPEVYFCCCEGNMCNERFTHLPEAG |
| | 12) with 6 additional aa at N- | GPEVTYEPPPTAPT |
| | terminus | |
| 1060 | N19Q mutant of Hybrid hu- | AETRECIYYNANWELERTQQSGLERCEGDQDKRLHCYASWRNSSGTIELVK |
| | ActRIIB-ECD (SEQ ID NO: | KGCWLDDFNCYDRQECVEKKDSPEVYFCCCEGNMCNERFTHLPEAGGPEV |
| | 13) with 1 additional aa at N- | TYEPPPTAPT |
| | terminus | |
| 1061 | N20Q mutant of Hybrid hu- | EAETRECIYYNANWELERTQQSGLERCEGDQDKRLHCYASWRNSSGTIELV |
| | ActRIIB-ECD (SEQ ID NO: | KKGCWLDDFNCYDRQECVEKKDSPEVYFCCCEGNMCNERFTHLPEAGGPE |
| | 13) with 2 additional aa at N- | VTYEPPPTAPT |
| | terminus | |
| 1062 | N21Q mutant of Hybrid hu- | GEAETRECIYYNANWELERTQQSGLERCEGDQDKRLHCYASWRNSSGTIEL |
| | ActRIIB-ECD (SEQ ID NO: | VKKGCWLDDFNCYDRQECVEKKDSPEVYFCCCEGNMCNERFTHLPEAGGP |
| | 13) with 3 additional aa at N- | EVTYEPPPTAPT |
| | terminus | |
| 1063 | N22Q mutant of Hybrid hu- | RGEAETRECIYYNANWELERTQQSGLERCEGDQDKRLHCYASWRNSSGTIE |
| | ActRIIB-ECD (SEQ ID NO: | LVKKGCWLDDFNCYDRQECVEKKDSPEVYFCCCEGNMCNERFTHLPEAGG |
| | 13) with 4 additional aa at N- | PEVTYEPPPTAPT |
| | terminus | |
| 1064 | N23Q mutant of Hybrid hu- | GRGEAETRECIYYNANWELERTQQSGLERCEGDQDKRLHCYASWRNSSGTI |
| | ActRIIB-ECD (SEQ ID NO: | ELVKKGCWLDDFNCYDRQECVEKKDSPEVYFCCCEGNMCNERFTHLPEAG |
| | 13) with 5 additional aa at N- | GPEVTYEPPPTAPT |
| | terminus | |
| 1065 | N24Q mutant of Hybrid hu- | SGRGEAETRECIYYNANWELERTQQSGLERCEGDQDKRLHCYASWRNSSGT |
| | ActRIIB-ECD (SEQ ID NO: | IELVKKGCWLDDFNCYDRQECVEKKDSPEVYFCCCEGNMCNERFTHLPEAG |
| | 13) with 6 additional aa at N- | GPEVTYEPPPTAPT |
| | terminus | |
| 1066 | N19Q mutant of Hybrid hu- | AETQECIYYNANWEKDRTQQTGVEPCYGDKDKRRHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 14) with 1 additional aa at N- | TYEPPPTAPT |
| | terminus | |
| 1067 | N20Q mutant of Hybrid hu- | EAETQECIYYNANWEKDRTQQTGVEPCYGDKDKRRHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 14) with 2 additional aa at N- | TYEPPPTAPT |
| | terminus | |
| 1068 | N21Q mutant of Hybrid hu- | GEAETQECIYYNANWEKDRTQQTGVEPCYGDKDKRRHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 14) with 3 additional aa at N- | VTYEPPPTAPT |
| | terminus | |
| 1069 | N22Q mutant of Hybrid hu- | RGEAETQECIYYNANWEKDRTQQTGVEPCYGDKDKRRHCYASWRNSSGTI |
| | ActRIIB-ECD (SEQ ID NO: | ELVKKGCWLDDFNCYDRQECVATEENPQVYFCCCEGNFCNERFTHLPEAGG |
| | 14) with 4 additional aa at N- | PEVTYEPPPTAPT |
| | terminus | |
| 1070 | N23Q mutant of Hybrid hu- | GRGEAETQECIYYNANWEKDRTQQTGVEPCYGDKDKRRHCYASWRNSSGT |
| | ActRIIB-ECD (SEQ ID NO: | IELVKKGCWLDDFNCYDRQECVATEENPQVYFCCCEGNFCNERFTHLPEAG |
| | 14) with 5 additional aa at N- | GPEVTYEPPPTAPT |
| | terminus | |
| 1071 | N24Q mutant of Hybrid hu- | SGRGEAETQECIYYNANWEKDRTQQTGVEPCYGDKDKRRHCYASWRNSSG |
| | ActRIIB-ECD (SEQ ID NO: | TIELVKKGCWLDDFNCYDRQECVATEENPQVYFCCCEGNFCNERFTHLPEA |
| | 14) with 6 additional aa at N- | GGPEVTYEPPPTAPT |
| | terminus | |
| 1072 | N19Q mutant of Hybrid hu- | AETQECIYYNANWEKDRTQQTGVEPCEGDQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 15) with 1 additional aa at N- | YEPPPTAPT |
| | terminus | |
| 1073 | N20Q mutant of Hybrid hu- | EAETQECIYYNANWEKDRTQQTGVEPCEGDQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 15) with 2 additional aa at N- | TYEPPPTAPT |
| | terminus | |
| 1074 | N21Q mutant of Hybrid hu- | GEAETQECIYYNANWEKDRTQQTGVEPCEGDQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 15) with 3 additional aa at N- | VTYEPPPTAPT |
| | terminus | |
| 1075 | N22Q mutant of Hybrid hu- | RGEAETQECIYYNANWEKDRTQQTGVEPCEGDQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 15) with 4 additional aa at N- | EVTYEPPPTAPT |
| | terminus | |
| 1076 | N23Q mutant of Hybrid hu- | GRGEAETQECIYYNANWEKDRTQQTGVEPCEGDQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 15) with 5 additional aa at N- | PEVTYEPPPTAPT |
| | terminus | |
| 1077 | N24Q mutant of Hybrid hu- | SGRGEAETQECIYYNANWEKDRTQQTGVEPCEGDQDKRLHCYASWRNSSG |
| | ActRIIB-ECD (SEQ ID NO: | TIELVKKGCWLDDFNCYDRQECVATEENPQVYFCCCEGNFCNERFTHLPEA |
| | 15) with 6 additional aa at N- | GGPEVTYEPPPTAPT |
| | terminus | |
| 1078 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 16) with 1 additional aa at N- | YEPPPTAPT |
| | terminus | |
| 1079 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 16) with 2 additional aa at N- | TYEPPPTAPT |
| | terminus | |
| 1080 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 16) with 3 additional aa at N- | VTYEPPPTAPT |
| | terminus | |
| 1081 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 16) with 4 additional aa at N- | EVTYEPPPTAPT |
| | terminus | |
| 1082 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDFNCYDRQECVATEENPQVYFCCCEGNFCNERFTHLPEAGG |
| | 16) with 5 additional aa at N- | PEVTYEPPPTAPT |
| | terminus | |
| 1083 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDFNCYDRQECVATEENPQVYFCCCEGNFCNERFTHLPEA |
| | 16) with 6 additional aa at N- | GGPEVTYEPPPTAPT |
| | terminus | |
| 1084 | N19Q mutant of Hybrid hu- | AETRECIYYNANWEKDRTQQTGVEPCEGDQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 17) with 1 additional aa at N- | YEPPPTAPT |
| | terminus | |
| 1085 | N20Q mutant of Hybrid hu- | EAETRECIYYNANWEKDRTQQTGVEPCEGDQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 17) with 2 additional aa at N- | TYEPPPTAPT |
| | terminus | |
| 1086 | N21Q mutant of Hybrid hu- | GEAETRECIYYNANWEKDRTQQTGVEPCEGDQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 17) with 3 additional aa at N- | VTYEPPPTAPT |
| | terminus | |
| 1087 | N22Q mutant of Hybrid hu- | RGEAETRECIYYNANWEKDRTQQTGVEPCEGDQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 17) with 4 additional aa at N- | EVTYEPPPTAPT |
| | terminus | |
| 1088 | N23Q mutant of Hybrid hu- | GRGEAETRECIYYNANWEKDRTQQTGVEPCEGDQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 17) with 5 additional aa at N- | PEVTYEPPPTAPT |
| | terminus | |
| 1089 | N24Q mutant of Hybrid hu- | SGRGEAETRECIYYNANWEKDRTQQTGVEPCEGDQDKRLHCYASWRNSSG |
| | ActRIIB-ECD (SEQ ID NO: | TIELVKKGCWLDDFNCYDRQECVATEENPQVYFCCCEGNFCNERFTHLPEA |
| | 17) with 6 additional aa at N- | GGPEVTYEPPPTAPT |
| | terminus | |
| 1090 | N19Q mutant of Hybrid hu- | AETQECIYYNANWEKDRTQQTGVEPCEGDQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 18) with 1 additional aa at N- | YEPPPTAPT |
| | terminus | |
| 1091 | N20Q mutant of Hybrid hu- | EAETQECIYYNANWEKDRTQQTGVEPCEGDQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 18) with 2 additional aa at N- | TYEPPPTAPT |
| | terminus | |
| 1092 | N21Q mutant of Hybrid hu- | GEAETQECIYYNANWEKDRTQQTGVEPCEGDQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 18) with 3 additional aa at N- | VTYEPPPTAPT |
| | terminus | |
| 1093 | N22Q mutant of Hybrid hu- | RGEAETQECIYYNANWEKDRTQQTGVEPCEGDQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 18) with 4 additional aa at N- | EVTYEPPPTAPT |
| | terminus | |
| 1094 | N23Q mutant of Hybrid hu- | GRGEAETQECIYYNANWEKDRTQQTGVEPCEGDQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 18) with 5 additional aa at N- | PEVTYEPPPTAPT |
| | terminus | |
| 1095 | N24Q mutant of Hybrid hu- | SGRGEAETQECIYYNANWEKDRTQQTGVEPCEGDQDKRLHCYASWRNSSG |
| | ActRIIB-ECD (SEQ ID NO: | TIELVKKGCWLDDFNCYDRQECVATEENPQVYFCCCEGNFCNERFTHLPEA |
| | 18) with 6 additional aa at N- | GGPEVTYEPPPTAPT |
| | terminus | |
| 1096 | N19Q mutant of Hybrid hu- | AETQECIYYNANWEKDRTQQTGLERCEGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 19) with 1 additional aa at N- | YEPPPTAPT |
| | terminus | |
| 1097 | N20Q mutant of Hybrid hu- | EAETQECIYYNANWEKDRTQQTGLERCEGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 19) with 2 additional aa at N- | TYEPPPTAPT |
| | terminus | |
| 1098 | N21Q mutant of Hybrid hu- | GEAETQECIYYNANWEKDRTQQTGLERCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 19) with 3 additional aa at N- | VTYEPPPTAPT |
| | terminus | |
| 1099 | N22Q mutant of Hybrid hu- | RGEAETQECIYYNANWEKDRTQQTGLERCEGEQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 19) with 4 additional aa at N- | EVTYEPPPTAPT |
| | terminus | |
| 1100 | N23Q mutant of Hybrid hu- | GRGEAETQECIYYNANWEKDRTQQTGLERCEGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 19) with 5 additional aa at N- | PEVTYEPPPTAPT |
| | terminus | |
| 1101 | N24Q mutant of Hybrid hu- | SGRGEAETQECIYYNANWEKDRTQQTGLERCEGEQDKRLHCYASWRNSSG |
| | ActRIIB-ECD (SEQ ID NO: | TIELVKKGCWLDDFNCYDRQECVATEENPQVYFCCCEGNFCNERFTHLPEA |
| | 19) with 6 additional aa at N- | GGPEVTYEPPPTAPT |
| | terminus | |
| 1102 | N19Q mutant of Hybrid hu- | AETQECIYYNANWEKDRTQQSGLERCEGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 20) with 1 additional aa at N- | YEPPPTAPT |
| | terminus | |
| 1103 | N20Q mutant of Hybrid hu- | EAETQECIYYNANWEKDRTQQSGLERCEGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 20) with 2 additional aa at N- | TYEPPPTAPT |
| | terminus | |
| 1104 | N21Q mutant of Hybrid hu- | GEAETQECIYYNANWEKDRTQQSGLERCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 20) with 3 additional aa at N- | VTYEPPPTAPT |
| | terminus | |
| 1105 | N22Q mutant of Hybrid hu- | RGEAETQECIYYNANWEKDRTQQSGLERCEGEQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 20) with 4 additional aa at N- | EVTYEPPPTAPT |
| | terminus | |
| 1106 | N23Q mutant of Hybrid hu- | GRGEAETQECIYYNANWEKDRTQQSGLERCEGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 20) with 5 additional aa at N- | PEVTYEPPPTAPT |
| | terminus | |
| 1107 | N24Q mutant of Hybrid hu- | SGRGEAETQECIYYNANWEKDRTQQSGLERCEGEQDKRLHCYASWRNSSG |
| | ActRIIB-ECD (SEQ ID NO: | TIELVKKGCWLDDFNCYDRQECVATEENPQVYFCCCEGNFCNERFTHLPEA |
| | 20) with 6 additional aa at N- | GGPEVTYEPPPTAPT |
| | terminus | |
| 1108 | N19Q mutant of Hybrid hu- | AETRECIYYNANWEKDRTQQSGLERCEGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 21) with 1 additional aa at N- | YEPPPTAPT |
| | terminus | |
| 1109 | N20Q mutant of Hybrid hu- | EAETRECIYYNANWEKDRTQQSGLERCEGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 21) with 2 additional aa at N- | TYEPPPTAPT |
| | terminus | |
| 1110 | N21Q mutant of Hybrid hu- | GEAETRECIYYNANWEKDRTQQSGLERCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 21) with 3 additional aa at N- | VTYEPPPTAPT |
| | terminus | |
| 1111 | N22Q mutant of Hybrid hu- | RGEAETRECIYYNANWEKDRTQQSGLERCEGEQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 21) with 4 additional aa at N- | EVTYEPPPTAPT |
| | terminus | |
| 1112 | N23Q mutant of Hybrid hu- | GRGEAETRECIYYNANWEKDRTQQSGLERCEGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 21) with 5 additional aa at N- | PEVTYEPPPTAPT |
| | terminus | |
| 1113 | N24Q mutant of Hybrid hu- | SGRGEAETRECIYYNANWEKDRTQQSGLERCEGEQDKRLHCYASWRNSSGT |
| | ActRIIB-ECD (SEQ ID NO: | IELVKKGCWLDDFNCYDRQECVATEENPQVYFCCCEGNFCNERFTHLPEAG |
| | 21) with 6 additional aa at N- | GPEVTYEPPPTAPT |
| | terminus | |
| 1114 | N19Q mutant of Hybrid hu- | AETQECIYYNANWELERTQQSGLERCEGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 22) with 1 additional aa at N- | YEPPPTAPT |
| | terminus | |
| 1115 | N20Q mutant of Hybrid hu- | EAETQECIYYNANWELERTQQSGLERCEGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 22) with 2 additional aa at N- | TYEPPPTAPT |
| | terminus | |
| 1116 | N21Q mutant of Hybrid hu- | GEAETQECIYYNANWELERTQQSGLERCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 22) with 3 additional aa at N- | VTYEPPPTAPT |
| | terminus | |
| 1117 | N22Q mutant of Hybrid hu- | RGEAETQECIYYNANWELERTQQSGLERCEGEQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 22) with 4 additional aa at N- | EVTYEPPPTAPT |
| | terminus | |
| 1118 | N23Q mutant of Hybrid hu- | GRGEAETQECIYYNANWELERTQQSGLERCEGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 22) with 5 additional aa at N- | PEVTYEPPPTAPT |
| | terminus | |
| 1119 | N24Q mutant of Hybrid hu- | SGRGEAETQECIYYNANWELERTQQSGLERCEGEQDKRLHCYASWRNSSGT |
| | ActRIIB-ECD (SEQ ID NO: | IELVKKGCWLDDFNCYDRQECVATEENPQVYFCCCEGNFCNERFTHLPEAG |
| | 22) with 6 additional aa at N- | GPEVTYEPPPTAPT |
| | terminus | |
| 1120 | N19Q mutant of Hybrid hu- | AETRECIYYNANWELERTQQSGLERCEGEQDKRLHCFATWRNSSGTIELVK |
| | ActRIIB-ECD (SEQ ID NO: | QGCWLDDFNCYDRQECVATEENPQVYFCCCEGNFCNERFTHLPEAGGPEVT |
| | 23) with 1 additional aa at N- | YEPPPTAPT |
| | terminus | |
| 1121 | N20Q mutant of Hybrid hu- | EAETRECIYYNANWELERTQQSGLERCEGEQDKRLHCFATWRNSSGTIELVK |
| | ActRIIB-ECD (SEQ ID NO: | QGCWLDDFNCYDRQECVATEENPQVYFCCCEGNFCNERFTHLPEAGGPEVT |
| | 23) with 2 additional aa at N- | YEPPPTAPT |
| | terminus | |
| 1122 | N21Q mutant of Hybrid hu- | GEAETRECIYYNANWELERTQQSGLERCEGEQDKRLHCFATWRNSSGTIELV |
| | ActRIIB-ECD (SEQ ID NO: | KQGCWLDDFNCYDRQECVATEENPQVYFCCCEGNFCNERFTHLPEAGGPEV |
| | 23) with 3 additional aa at N- | TYEPPPTAPT |
| | terminus | |
| 1123 | N22Q mutant of Hybrid hu- | RGEAETRECIYYNANWELERTQQSGLERCEGEQDKRLHCFATWRNSSGTIEL |
| | ActRIIB-ECD (SEQ ID NO: | VKQGCWLDDFNCYDRQECVATEENPQVYFCCCEGNFCNERFTHLPEAGGPE |
| | 23) with 4 additional aa at N- | VTYEPPPTAPT |
| | terminus | |
| 1124 | N23Q mutant of Hybrid hu- | GRGEAETRECIYYNANWELERTQQSGLERCEGEQDKRLHCFATWRNSSGTI |
| | ActRIIB-ECD (SEQ ID NO: | ELVKQGCWLDDFNCYDRQECVATEENPQVYFCCCEGNFCNERFTHLPEAGG |
| | 23) with 5 additional aa at N- | PEVTYEPPPTAPT |
| | terminus | |
| 1125 | N24Q mutant of Hybrid hu- | SGRGEAETRECIYYNANWELERTQQSGLERCEGEQDKRLHCFATWRNSSGTI |
| | ActRIIB-ECD (SEQ ID NO: | ELVKQGCWLDDFNCYDRQECVATEENPQVYFCCCEGNFCNERFTHLPEAGG |
| | 23) with 6 additional aa at N- | PEVTYEPPPTAPT |
| | terminus | |
| 1126 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDINCYDRQECVATEENPQVYFCCCEGNFCNERFTHLPEAGGPEVT |
| | 24) with 1 additional aa at N- | YEPPPTAPT |
| | terminus | |
| 1127 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDINCYDRQECVATEENPQVYFCCCEGNFCNERFTHLPEAGGPEV |
| | 24) with 2 additional aa at N- | TYEPPPTAPT |
| | terminus | |
| 1128 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDINCYDRQECVATEENPQVYFCCCEGNFCNERFTHLPEAGGPE |
| | 24) with 3 additional aa at N- | VTYEPPPTAPT |
| | terminus | |
| 1129 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDINCYDRQECVATEENPQVYFCCCEGNFCNERFTHLPEAGGP |
| | 24) with 4 additional aa at N- | EVTYEPPPTAPT |
| | terminus | |
| 1130 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDINCYDRQECVATEENPQVYFCCCEGNFCNERFTHLPEAGG |
| | 24) with 5 additional aa at N- | PEVTYEPPPTAPT |
| | terminus | |
| 1131 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDINCYDRQECVATEENPQVYFCCCEGNFCNERFTHLPEAG |
| | 24) with 6 additional aa at N- | GPEVTYEPPPTAPT |
| | terminus | |
| 1132 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATKENPQVYFCCCEGNFCNERFTHLPEAGGPEVT |
| | 25) with 1 additional aa at N- | YEPPPTAPT |
| | terminus | |
| 1133 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATKENPQVYFCCCEGNFCNERFTHLPEAGGPE |
| | 25) with 2 additional aa at N- | VTYEPPPTAPT |
| | terminus | |
| 1134 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATKENPQVYFCCCEGNFCNERFTHLPEAGGP |
| | 25) with 3 additional aa at N- | EVTYEPPPTAPT |
| | terminus | |
| 1135 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATKENPQVYFCCCEGNFCNERFTHLPEAGG |
| | 25) with 4 additional aa at N- | PEVTYEPPPTAPT |
| | terminus | |
| 1136 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATKENPQVYFCCCEGNFCNERFTHLPEAG |
| | 25) with 5 additional aa at N- | GPEVTYEPPPTAPT |
| | terminus | |
| 1137 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATKENPQVYFCCCEGNFCNERFTHLPEAG |
| | 25) with 6 additional aa at N- | GPEVTYEPPPTAPT |
| | terminus | |
| 1138 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDINCYDRQECVATEENPQVYFCCCEGNFCNERFTHLPEAGGPEVT |
| | 26) with 1 additional aa at N- | YEPPPTAPT |
| | terminus | |
| 1139 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDINCYDRQECVATEENPQVYFCCCEGNFCNERFTHLPEAGGPEV |
| | 26) with 2 additional aa at N- | TYEPPPTAPT |
| | terminus | |
| 1140 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDINCYDRQECVATEENPQVYFCCCEGNFCNERFTHLPEAGGPE |
| | 26) with 3 additional aa at N- | VTYEPPPTAPT |
| | terminus | |
| 1141 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDINCYDRQECVATEENPQVYFCCCEGNFCNERFTHLPEAGGP |
| | 26) with 4 additional aa at N- | EVTYEPPPTAPT |
| | terminus | |
| 1142 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDINCYDRQECVATEENPQVYFCCCEGNFCNERFTHLPEAGG |
| | 26) with 5 additional aa at N- | PEVTYEPPPTAPT |
| | terminus | |
| 1143 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDINCYDRQECVATEENPQVYFCCCEGNFCNERFTHLPEAG |
| | 26) with 6 additional aa at N- | GPEVTYEPPPTAPT |
| | terminus | |
| 1144 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDINCYDRQECVATKENPQVYFCCCEGNFCNERFTHLPEAGGPEVT |
| | 27) with 1 additional aa at N- | YEPPPTAPT |
| | terminus | |
| 1145 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDINCYDRQECVATKENPQVYFCCCEGNFCNERFTHLPEAGGPEV |
| | 27) with 2 additional aa at N- | TYEPPPTAPT |
| | terminus | |
| 1146 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDINCYDRQECVATKENPQVYFCCCEGNFCNERFTHLPEAGGPE |
| | 27) with 3 additional aa at N- | VTYEPPPTAPT |
| | terminus | |
| 1147 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDINCYDRQECVATKENPQVYFCCCEGNFCNERFTHLPEAGGP |
| | 27) with 4 additional aa at N- | EVTYEPPPTAPT |
| | terminus | |
| 1148 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDINCYDRQECVATKENPQVYFCCCEGNFCNERFTHLPEAGG |
| | 27) with 5 additional aa at N- | PEVTYEPPPTAPT |
| | terminus | |
| 1149 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDINCYDRQECVATKENPQVYFCCCEGNFCNERFTHLPEAG |
| | 27) with 6 additional aa at N- | GPEVTYEPPPTAPT |
| | terminus | |
| 1150 | N19Q mutant of Hybrid hu- | AETRECIYYNANWELERTQQSGLERCEGDKDKRLHCYASWRNSSGTIELVK |
| | ActRIIB-ECD (SEQ ID NO: | KGCWLDDINCYDRQECVATKENPQVYFCCCEGNFCNERFTHLPEAGGPEVT |
| | 28) with 1 additional aa at N- | YEPPPTAPT |
| | terminus | |
| 1151 | N20Q mutant of Hybrid hu- | EAETRECIYYNANWELERTQQSGLERCEGDKDKRLHCYASWRNSSGTIELV |
| | ActRIIB-ECD (SEQ ID NO: | KKGCWLDDINCYDRQECVATKENPQVYFCCCEGNFCNERFTHLPEAGGPEV |
| | 28) with 2 additional aa at N- | TYEPPPTAPT |
| | terminus | |
| 1152 | N21Q mutant of Hybrid hu- | GEAETRECIYYNANWELERTQQSGLERCEGDKDKRLHCYASWRNSSGTIEL |
| | ActRIIB-ECD (SEQ ID NO: | VKKGCWLDDINCYDRQECVATKENPQVYFCCCEGNFCNERFTHLPEAGGPE |
| | 28) with 3 additional aa at N- | VTYEPPPTAPT |
| | terminus | |
| 1153 | N22Q mutant of Hybrid hu- | RGEAETRECIYYNANWELERTQQSGLERCEGDKDKRLHCYASWRNSSGTIE |
| | ActRIIB-ECD (SEQ ID NO: | LVKKGCWLDDINCYDRQECVATKENPQVYFCCCEGNFCNERFTHLPEAGGP |
| | 28) with 4 additional aa at N- | EVTYEPPPTAPT |
| | terminus | |
| 1154 | N23Q mutant of Hybrid hu- | GRGEAETRECIYYNANWELERTQQSGLERCEGDKDKRLHCYASWRNSSGTI |
| | ActRIIB-ECD (SEQ ID NO: | ELVKKGCWLDDINCYDRQECVATKENPQVYFCCCEGNFCNERFTHLPEAGG |
| | 28) with 5 additional aa at N- | PEVTYEPPPTAPT |
| | terminus | |
| 1155 | N24Q mutant of Hybrid hu- | SGRGEAETRECIYYNANWELERTQQSGLERCEGDKDKRLHCYASWRNSSGT |
| | ActRIIB-ECD (SEQ ID NO: | IELVKKGCWLDDINCYDRQECVATKENPQVYFCCCEGNFCNERFTHLPEAG |
| | 28) with 6 additional aa at N- | GPEVTYEPPPTAPT |
| | terminus | |
| 1156 | N19Q mutant of Hybrid hu- | AETRECIYYNANWELERTQQSGLERCEGDQDKRLHCYASWRNSSGTIELVK |
| | ActRIIB-ECD (SEQ ID NO: | KGCWLDDINCYDRQECVATKENPQVYFCCCEGNFCNERFTHLPEAGGPEVT |
| | 29) with 1 additional aa at N- | YEPPPTAPT |
| | terminus | |
| 1157 | N20Q mutant of Hybrid hu- | EAETRECIYYNANWELERTQQSGLERCEGDQDKRLHCYASWRNSSGTIELV |
| | ActRIIB-ECD (SEQ ID NO: | KKGCWLDDINCYDRQECVATKENPQVYFCCCEGNFCNERFTHLPEAGGPEV |
| | 29) with 2 additional aa at N- | TYEPPPTAPT |
| | terminus | |
| 1158 | N21Q mutant of Hybrid hu- | GEAETRECIYYNANWELERTQQSGLERCEGDQDKRLHCYASWRNSSGTIEL |
| | ActRIIB-ECD (SEQ ID NO: | VKKGCWLDDINCYDRQECVATKENPQVYFCCCEGNFCNERFTHLPEAGGPE |
| | 29) with 3 additional aa at N- | VTYEPPPTAPT |
| | terminus | |
| 1159 | N22Q mutant of Hybrid hu- | RGEAETRECIYYNANWELERTQQSGLERCEGDQDKRLHCYASWRNSSGTIE |
| | ActRIIB-ECD (SEQ ID NO: | LVKKGCWLDDINCYDRQECVATKENPQVYFCCCEGNFCNERFTHLPEAGGP |
| | 29) with 4 additional aa at N- | EVTYEPPPTAPT |
| | terminus | |
| 1160 | N23Q mutant of Hybrid hu- | GRGEAETRECIYYNANWELERTQQSGLERCEGDQDKRLHCYASWRNSSGTI |
| | ActRIIB-ECD (SEQ ID NO: | ELVKKGCWLDDINCYDRQECVATKENPQVYFCCCEGNFCNERFTHLPEAGG |
| | 29) with 5 additional aa at N- | PEVTYEPPPTAPT |
| | terminus | |
| 1161 | N24Q mutant of Hybrid hu- | SGRGEAETRECIYYNANWELERTQQSGLERCEGDQDKRLHCYASWRNSSGT |
| | ActRIIB-ECD (SEQ ID NO: | IELVKKGCWLDDINCYDRQECVATKENPQVYFCCCEGNFCNERFTHLPEAG |
| | 29) with 6 additional aa at N- | GPEVTYEPPPTAPT |
| | terminus | |
| 1162 | N19Q mutant of Hybrid hu- | AETRECIYYNANWELERTQQSGLERCEGDQDKRLHCYASWRNSSGTIELVK |
| | ActRIIB-ECD (SEQ ID NO: | KGCWLDDFNCYDRQECVATKENPQVYFCCCEGNFCNERFTHLPEAGGPEVT |
| | 30) with 1 additional aa at N- | YEPPPTAPT |
| | terminus | |
| 1163 | N20Q mutant of Hybrid hu- | EAETRECIYYNANWELERTQQSGLERCEGDQDKRLHCYASWRNSSGTIELV |
| | ActRIIB-ECD (SEQ ID NO: | KKGCWLDDFNCYDRQECVATKENPQVYFCCCEGNFCNERFTHLPEAGGPE |
| | 30) with 2 additional aa at N- | VTYEPPPTAPT |
| | terminus | |
| 1164 | N21Q mutant of Hybrid hu- | GEAETRECIYYNANWELERTQQSGLERCEGDQDKRLHCYASWRNSSGTIEL |
| | ActRIIB-ECD (SEQ ID NO: | VKKGCWLDDFNCYDRQECVATKENPQVYFCCCEGNFCNERFTHLPEAGGP |
| | 30) with 3 additional aa at N- | EVTYEPPPTAPT |
| | terminus | |
| 1165 | N22Q mutant of Hybrid hu- | RGEAETRECIYYNANWELERTQQSGLERCEGDQDKRLHCYASWRNSSGTIE |
| | ActRIIB-ECD (SEQ ID NO: | LVKKGCWLDDFNCYDRQECVATKENPQVYFCCCEGNFCNERFTHLPEAGG |
| | 30) with 4 additional aa at N- | PEVTYEPPPTAPT |
| | terminus | |
| 1166 | N23Q mutant of Hybrid hu- | GRGEAETRECIYYNANWELERTQQSGLERCEGDQDKRLHCYASWRNSSGTI |
| | ActRIIB-ECD (SEQ ID NO: | ELVKKGCWLDDFNCYDRQECVATKENPQVYFCCCEGNFCNERFTHLPEAG |
| | 30) with 5 additional aa at N- | GPEVTYEPPPTAPT |
| | terminus | |
| 1167 | N24Q mutant of Hybrid hu- | SGRGEAETRECIYYNANWELERTQQSGLERCEGDQDKRLHCYASWRNSSGT |
| | ActRIIB-ECD (SEQ ID NO: | IELVKKGCWLDDFNCYDRQECVATKENPQVYFCCCEGNFCNERFTHLPEAG |
| | 30) with 6 additional aa at N- | GPEVTYEPPPTAPT |
| | terminus | |
| 1168 | N19Q mutant of Hybrid hu- | AETRECIYYNANWELERTQQSGLERCEGDQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 31) with 1 additional aa at N- | YEPPPTAPT |
| | terminus | |
| 1169 | N20Q mutant of Hybrid hu- | EAETRECIYYNANWELERTQQSGLERCEGDQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 31) with 2 additional aa at N- | TYEPPPTAPT |
| | terminus | |
| 1170 | N21Q mutant of Hybrid hu- | GEAETRECIYYNANWELERTQQSGLERCEGDQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 31) with 3 additional aa at N- | VTYEPPPTAPT |
| | terminus | |
| 1171 | N22Q mutant of Hybrid hu- | RGEAETRECIYYNANWELERTQQSGLERCEGDQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 31) with 4 additional aa at N- | EVTYEPPPTAPT |
| | terminus | |
| 1172 | N23Q mutant of Hybrid hu- | GRGEAETRECIYYNANWELERTQQSGLERCEGDQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 31) with 5 additional aa at N- | PEVTYEPPPTAPT |
| | terminus | |
| 1173 | N24Q mutant of Hybrid hu- | SGRGEAETRECIYYNANWELERTQQSGLERCEGDQDKRLHCYASWRNSSGT |
| | ActRIIB-ECD (SEQ ID NO: | IELVKKGCWLDDFNCYDRQECVATEENPQVYFCCCEGNFCNERFTHLPEAG |
| | 31) with 6 additional aa at N- | GPEVTYEPPPTAPT |
| | terminus | |
| 1174 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDINCYDRQECVEKKDSPEVYFCCCEGNFCNERFTHLPEAGGPEVT |
| | 32) with 1 additional aa at N- | YEPPPTAPT |
| | terminus | |
| 1175 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDINCYDRQECVEKKDSPEVYFCCCEGNFCNERFTHLPEAGGPEV |
| | 32) with 2 additional aa at N- | TYEPPPTAPT |
| | terminus | |
| 1176 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDINCYDRQECVEKKDSPEVYFCCCEGNFCNERFTHLPEAGGPE |
| | 32) with 3 additional aa at N- | VTYEPPPTAPT |
| | terminus | |
| 1177 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDINCYDRQECVEKKDSPEVYFCCCEGNFCNERFTHLPEAGGP |
| | 32) with 4 additional aa at N- | EVTYEPPPTAPT |
| | terminus | |
| 1178 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDINCYDRQECVEKKDSPEVYFCCCEGNFCNERFTHLPEAGG |
| | 32) with 5 additional aa at N- | PEVTYEPPPTAPT |
| | terminus | |
| 1179 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDINCYDRQECVEKKDSPEVYFCCCEGNFCNERFTHLPEAG |
| | 32) with 6 additional aa at N- | GPEVTYEPPPTAPT |
| | terminus | |
| 1180 | N19Q mutant of Hybrid hu- | AETRECIFFNANWEKDRTQQTGVEPCEGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 33) with 1 additional aa at N- | YEPPPTAPT |
| | terminus | |
| 1181 | N20Q mutant of Hybrid hu- | EAETRECIFFNANWEKDRTQQTGVEPCEGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 33) with 2 additional aa at N- | TYEPPPTAPT |
| | terminus | |
| 1182 | N21Q mutant of Hybrid hu- | GEAETRECIFFNANWEKDRTQQTGVEPCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 33) with 3 additional aa at N- | VTYEPPPTAPT |
| | terminus | |
| 1183 | N22Q mutant of Hybrid hu- | RGEAETRECIFFNANWEKDRTQQTGVEPCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 33) with 4 additional aa at N- | VTYEPPPTAPT |
| | terminus | |
| 1184 | N23Q mutant of Hybrid hu- | GRGEAETRECIFFNANWEKDRTQQTGVEPCEGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 33) with 5 additional aa at N- | PEVTYEPPPTAPT |
| | terminus | |
| 1185 | N24Q mutant of Hybrid hu- | SGRGEAETRECIFFNANWEKDRTQQTGVEPCEGEQDKRLHCYASWRNSSGT |
| | ActRIIB-ECD (SEQ ID NO: | IELVKKGCWLDDFNCYDRQECVATEENPQVYFCCCEGNFCNERFTHLPEAG |
| | 33) with 6 additional aa at N- | GPEVTYEPPPTAPT |
| | terminus | |
| 1186 | N19Q mutant of Hybrid hu- | AETRECIYYNANWELERTQQSGLERCEGEQDKRLHCFATWKNISGSIELVKQ |
| | ActRIIB-ECD (SEQ ID NO: | GCWLDDFNCYDRQECVATEENPQVYFCCCEGNFCNERFTHLPEAGGPEVTY |
| | 34) with 1 additional aa at N- | EPPPTAPT |
| | terminus | |
| 1187 | N20Q mutant of Hybrid hu- | EAETRECIYYNANWELERTQQSGLERCEGEQDKRLHCFATWKNISGSIELVK |
| | ActRIIB-ECD (SEQ ID NO: | QGCWLDDFNCYDRQECVATEENPQVYFCCCEGNFCNERFTHLPEAGGPEVT |
| | 34) with 2 additional aa at N- | YEPPPTAPT |
| | terminus | |
| 1188 | N21Q mutant of Hybrid hu- | GEAETRECIYYNANWELERTQQSGLERCEGEQDKRLHCFATWKNISGSIELV |
| | ActRIIB-ECD (SEQ ID NO: | KQGCWLDDFNCYDRQECVATEENPQVYFCCCEGNFCNERFTHLPEAGGPEV |
| | 34) with 3 additional aa at N- | TYEPPPTAPT |
| | terminus | |
| 1189 | N22Q mutant of Hybrid hu- | RGEAETRECIYYNANWELERTQQSGLERCEGEQDKRLHCFATWKNISGSIEL |
| | ActRIIB-ECD (SEQ ID NO: | VKQGCWLDDFNCYDRQECVATEENPQVYFCCCEGNFCNERFTHLPEAGGPE |
| | 34) with 4 additional aa at N- | VTYEPPPTAPT |
| | terminus | |
| 1190 | N23Q mutant of Hybrid hu- | GRGEAETRECIYYNANWELERTQQSGLERCEGEQDKRLHCFATWKNISGSIE |
| | ActRIIB-ECD (SEQ ID NO: | LVKQGCWLDDFNCYDRQECVATEENPQVYFCCCEGNFCNERFTHLPEAGGP |
| | 34) with 5 additional aa at N- | EVTYEPPPTAPT |
| | terminus | |
| 1191 | N24Q mutant of Hybrid hu- | SGRGEAETRECIYYNANWELERTQQSGLERCEGEQDKRLHCFATWKNISGSI |
| | ActRIIB-ECD (SEQ ID NO: | ELVKQGCWLDDFNCYDRQECVATEENPQVYFCCCEGNFCNERFTHLPEAGG |
| | 34) with 6 additional aa at N- | PEVTYEPPPTAPT |
| | terminus | |
| 1192 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRTDCVATEENPQVYFCCCEGNFCNERFTHLPEAGGPEVT |
| | 35) with 1 additional aa at N- | YEPPPTAPT |
| | terminus | |
| 1193 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRTDCVATEENPQVYFCCCEGNFCNERFTHLPEAGGPEV |
| | 35) with 2 additional aa at N- | TYEPPPTAPT |
| | terminus | |
| 1194 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRTDCVATEENPQVYFCCCEGNFCNERFTHLPEAGGPE |
| | 35) with 3 additional aa at N- | VTYEPPPTAPT |
| | terminus | |
| 1195 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRTDCVATEENPQVYFCCCEGNFCNERFTHLPEAGGP |
| | 35) with 4 additional aa at N- | EVTYEPPPTAPT |
| | terminus | |
| 1196 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRTDCVATEENPQVYFCCCEGNFCNERFTHLPEAGG |
| | 35) with 5 additional aa at N- | PEVTYEPPPTAPT |
| | terminus | |
| 1197 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRTDCVATEENPQVYFCCCEGNFCNERFTHLPEAG |
| | 35) with 6 additional aa at N- | GPEVTYEPPPTAPT |
| | terminus | |
| 1198 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATEENPQVYFCCCEGNMCNERFTHLPEAGGPEV |
| | 36) with 1 additional aa at N- | TYEPPPTAPT |
| | terminus | |
| 1199 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATEENPQVYFCCCEGNMCNERFTHLPEAGGPE |
| | 36) with 2 additional aa at N- | VTYEPPPTAPT |
| | terminus | |
| 1200 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATEENPQVYFCCCEGNMCNERFTHLPEAGGP |
| | 36) with 3 additional aa at N- | EVTYEPPPTAPT |
| | terminus | |
| 1201 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATEENPQVYFCCCEGNMCNERFTHLPEAGG |
| | 36) with 4 additional aa at N- | PEVTYEPPPTAPT |
| | terminus | |
| 1202 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATEENPQVYFCCCEGNMCNERFTHLPEAG |
| | 36) with 5 additional aa at N- | GPEVTYEPPPTAPT |
| | terminus | |
| 1203 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATEENPQVYFCCCEGNMCNERFTHLPEAG |
| | 36) with 6 additional aa at N- | GPEVTYEPPPTAPT |
| | terminus | |
| 1204 | N19Q mutant of Hybrid hu- | AETRECIYYNANWELERTQQSGLERCEGDQDKRLHCYASWRNSSGTIELVK |
| | ActRIIB-ECD (SEQ ID NO: | KGCWLDDINCYDRQECVATEENPQVYFCCCEGNFCNERFTHLPEAGGPEVT |
| | 37) with 1 additional aa at N- | YEPPPTAPT |
| | terminus | |
| 1205 | N20Q mutant of Hybrid hu- | EAETRECIYYNANWELERTQQSGLERCEGDQDKRLHCYASWRNSSGTIELV |
| | ActRIIB-ECD (SEQ ID NO: | KKGCWLDDINCYDRQECVATEENPQVYFCCCEGNFCNERFTHLPEAGGPEV |
| | 37) with 2 additional aa at N- | TYEPPPTAPT |
| | terminus | |
| 1206 | N21Q mutant of Hybrid hu- | GEAETRECIYYNANWELERTQQSGLERCEGDQDKRLHCYASWRNSSGTIEL |
| | ActRIIB-ECD (SEQ ID NO: | VKKGCWLDDINCYDRQECVATEENPQVYFCCCEGNFCNERFTHLPEAGGPE |
| | 37) with 3 additional aa at N- | VTYEPPPTAPT |
| | terminus | |
| 1207 | N22Q mutant of Hybrid hu- | RGEAETRECIYYNANWELERTQQSGLERCEGDQDKRLHCYASWRNSSGTIE |
| | ActRIIB-ECD (SEQ ID NO: | LVKKGCWLDDINCYDRQECVATEENPQVYFCCCEGNFCNERFTHLPEAGGP |
| | 37) with 4 additional aa at N- | EVTYEPPPTAPT |
| | terminus | |
| 1208 | N23Q mutant of Hybrid hu- | GRGEAETRECIYYNANWELERTQQSGLERCEGDQDKRLHCYASWRNSSGTI |
| | ActRIIB-ECD (SEQ ID NO: | ELVKKGCWLDDINCYDRQECVATEENPQVYFCCCEGNFCNERFTHLPEAGG |
| | 37) with 5 additional aa at N- | PEVTYEPPPTAPT |
| | terminus | |
| 1209 | N24Q mutant of Hybrid hu- | SGRGEAETRECIYYNANWELERTQQSGLERCEGDQDKRLHCYASWRNSSGT |
| | ActRIIB-ECD (SEQ ID NO: | IELVKKGCWLDDINCYDRQECVATEENPQVYFCCCEGNFCNERFTHLPEAG |
| | 37) with 6 additional aa at N- | GPEVTYEPPPTAPT |
| | terminus | |
| 1210 | N19Q mutant of Hybrid hu- | AETQECLFFNANWEKDRTQQSGVEPCYGDKDKRRHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 51) with 1 additional aa at N- | YEPPPTAPT |
| | terminus | |
| 1211 | N20Q mutant of Hybrid hu- | EAETQECLFFNANWEKDRTQQSGVEPCYGDKDKRRHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 51) with 2 additional aa at N- | TYEPPPTAPT |
| | terminus | |
| 1212 | N21Q mutant of Hybrid hu- | GEAETQECLFFNANWEKDRTQQSGVEPCYGDKDKRRHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 51) with 3 additional aa at N- | VTYEPPPTAPT |
| | terminus | |
| 1213 | N22Q mutant of Hybrid hu- | RGEAETQECLFFNANWEKDRTQQSGVEPCYGDKDKRRHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 51) with 4 additional aa at N- | EVTYEPPPTAPT |
| | terminus | |
| 1214 | N23Q mutant of Hybrid hu- | GRGEAETQECLFFNANWEKDRTQQSGVEPCYGDKDKRRHCYASWRNSSGT |
| | ActRIIB-ECD (SEQ ID NO: | IELVKKGCWLDDFNCYDRQECVATEENPQVYFCCCEGNFCNERFTHLPEAG |
| | 51) with 5 additional aa at N- | GPEVTYEPPPTAPT |
| | terminus | |
| 1215 | N24Q mutant of Hybrid hu- | SGRGEAETQECLFFNANWEKDRTQQSGVEPCYGDKDKRRHCYASWRNSSG |
| | ActRIIB-ECD (SEQ ID NO: | TIELVKKGCWLDDFNCYDRQECVATEENPQVYFCCCEGNFCNERFTHLPEA |
| | 51) with 6 additional aa at N- | GGPEVTYEPPPTAPT |
| | terminus | |
| 1216 | N19Q mutant of Hybrid hu- | AETQECLFFNANWEKDRTQQSGVEPCEGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 52) with 1 additional aa at N- | YEPPPTAPT |
| | terminus | |
| 1217 | N20Q mutant of Hybrid hu- | EAETQECLFFNANWEKDRTQQSGVEPCEGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 52) with 2 additional aa at N- | TYEPPPTAPT |
| | terminus | |
| 1218 | N21Q mutant of Hybrid hu- | GEAETQECLFFNANWEKDRTQQSGVEPCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 52) with 3 additional aa at N- | VTYEPPPTAPT |
| | terminus | |
| 1219 | N22Q mutant of Hybrid hu- | RGEAETQECLFFNANWEKDRTQQSGVEPCEGEQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 52) with 4 additional aa at N- | EVTYEPPPTAPT |
| | terminus | |
| 1220 | N23Q mutant of Hybrid hu- | GRGEAETQECLFFNANWEKDRTQQSGVEPCEGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 52) with 5 additional aa at N- | PEVTYEPPPTAPT |
| | terminus | |
| 1221 | N24Q mutant of Hybrid hu- | SGRGEAETQECLFFNANWEKDRTQQSGVEPCEGEQDKRLHCYASWRNSSGT |
| | ActRIIB-ECD (SEQ ID NO: | IELVKKGCWLDDFNCYDRQECVATEENPQVYFCCCEGNFCNERFTHLPEAG |
| | 52) with 6 additional aa at N- | GPEVTYEPPPTAPT |
| | terminus | |
| 1222 | N19Q mutant of Hybrid hu- | AETRECLFFNANWEKDRTQQSGVEPCEGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 53) with 1 additional aa at N- | YEPPPTAPT |
| | terminus | |
| 1223 | N20Q mutant of Hybrid hu- | EAETRECLFFNANWEKDRTQQSGVEPCEGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 53) with 2 additional aa at N- | TYEPPPTAPT |
| | terminus | |
| 1224 | N21Q mutant of Hybrid hu- | GEAETRECLFFNANWEKDRTQQSGVEPCEGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 53) with 3 additional aa at N- | VTYEPPPTAPT |
| | terminus | |
| 1225 | N22Q mutant of Hybrid hu- | RGEAETRECLFFNANWEKDRTQQSGVEPCEGEQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 53) with 4 additional aa at N- | EVTYEPPPTAPT |
| | terminus | |
| 1226 | N23Q mutant of Hybrid hu- | GRGEAETRECLFFNANWEKDRTQQSGVEPCEGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 53) with 5 additional aa at N- | PEVTYEPPPTAPT |
| | terminus | |
| 1227 | N24Q mutant of Hybrid hu- | SGRGEAETRECLFFNANWEKDRTQQSGVEPCEGEQDKRLHCYASWRNSSGT |
| | ActRIIB-ECD (SEQ ID NO: | IELVKKGCWLDDFNCYDRQECVATEENPQVYFCCCEGNFCNERFTHLPEAG |
| | 53) with 6 additional aa at N- | GPEVTYEPPPTAPT |
| | terminus | |
| 1228 | N19Q mutant of Hybrid hu- | AETQECLFFNANWEKDRTQQSGVEPCYGEQDKRLHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 54) with 1 additional aa at N- | YEPPPTAPT |
| | terminus | |
| 1229 | N20Q mutant of Hybrid hu- | EAETQECLFFNANWEKDRTQQSGVEPCYGEQDKRLHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 54) with 2 additional aa at N- | TYEPPPTAPT |
| | terminus | |
| 1230 | N21Q mutant of Hybrid hu- | GEAETQECLFFNANWEKDRTQQSGVEPCYGEQDKRLHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 54) with 3 additional aa at N- | VTYEPPPTAPT |
| | terminus | |
| 1231 | N22Q mutant of Hybrid hu- | RGEAETQECLFFNANWEKDRTQQSGVEPCYGEQDKRLHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 54) with 4 additional aa at N- | EVTYEPPPTAPT |
| | terminus | |
| 1232 | N23Q mutant of Hybrid hu- | GRGEAETQECLFFNANWEKDRTQQSGVEPCYGEQDKRLHCYASWRNSSGTI |
| | ActRIIB-ECD (SEQ ID NO: | ELVKKGCWLDDFNCYDRQECVATEENPQVYFCCCEGNFCNERFTHLPEAGG |
| | 54) with 5 additional aa at N- | PEVTYEPPPTAPT |
| | terminus | |
| 1233 | N24Q mutant of Hybrid hu- | SGRGEAETQECLFFNANWEKDRTQQSGVEPCYGEQDKRLHCYASWRNSSG |
| | ActRIIB-ECD (SEQ ID NO: | TIELVKKGCWLDDFNCYDRQECVATEENPQVYFCCCEGNFCNERFTHLPEA |
| | 54) with 6 additional aa at N- | GGPEVTYEPPPTAPT |
| | terminus | |
| 1234 | N19Q mutant of Hybrid hu- | AETRECLFFNANWEKDRTQQSGVEPCYGDKDKRRHCYASWRNSSGTIELVK |
| | ActRIIB-ECD (SEQ ID NO: | KGCWLDDFNCYDRQECVATEENPQVYFCCCEGNFCNERFTHLPEAGGPEVT |
| | 55) with 1 additional aa at N- | YEPPPTAPT |
| | terminus | |
| 1235 | N20Q mutant of Hybrid hu- | EAETRECLFFNANWEKDRTQQSGVEPCYGDKDKRRHCYASWRNSSGTIELV |
| | ActRIIB-ECD (SEQ ID NO: | KKGCWLDDFNCYDRQECVATEENPQVYFCCCEGNFCNERFTHLPEAGGPEV |
| | 55) with 2 additional aa at N- | TYEPPPTAPT |
| | terminus | |
| 1236 | N21Q mutant of Hybrid hu- | GEAETRECLFFNANWEKDRTQQSGVEPCYGDKDKRRHCYASWRNSSGTIEL |
| | ActRIIB-ECD (SEQ ID NO: | VKKGCWLDDFNCYDRQECVATEENPQVYFCCCEGNFCNERFTHLPEAGGPE |
| | 55) with 3 additional aa at N- | VTYEPPPTAPT |
| | terminus | |
| 1237 | N22Q mutant of Hybrid hu- | RGEAETRECLFFNANWEKDRTQQSGVEPCYGDKDKRRHCYASWRNSSGTIE |
| | ActRIIB-ECD (SEQ ID NO: | LVKKGCWLDDFNCYDRQECVATEENPQVYFCCCEGNFCNERFTHLPEAGGP |
| | 55) with 4 additional aa at N- | EVTYEPPPTAPT |
| | terminus | |
| 1238 | N23Q mutant of Hybrid hu- | GRGEAETRECLFFNANWEKDRTQQSGVEPCYGDKDKRRHCYASWRNSSGTI |
| | ActRIIB-ECD (SEQ ID NO: | ELVKKGCWLDDFNCYDRQECVATEENPQVYFCCCEGNFCNERFTHLPEAGG |
| | 55) with 5 additional aa at N- | PEVTYEPPPTAPT |
| | terminus | |
| 1239 | N24Q mutant of Hybrid hu- | SGRGEAETRECLFFNANWEKDRTQQSGVEPCYGDKDKRRHCYASWRNSSG |
| | ActRIIB-ECD (SEQ ID NO: | TIELVKKGCWLDDFNCYDRQECVATEENPQVYFCCCEGNFCNERFTHLPEA |
| | 55) with 6 additional aa at N- | GGPEVTYEPPPTAPT |
| | terminus | |
| 1240 | N19Q mutant of Hybrid hu- | AETRECLFFNANWEKDRTQQTGVEPCYGDKDKRRHCFATWKNISGSIEIVK |
| | ActRIIB-ECD (SEQ ID NO: | QGCWLDDFNCYDRQECVATEENPQVYFCCCEGNFCNERFTHLPEAGGPEVT |
| | 56) with 1 additional aa at N- | YEPPPTAPT |
| | terminus | |
| 1241 | N20Q mutant of Hybrid hu- | EAETRECLFFNANWEKDRTQQTGVEPCYGDKDKRRHCFATWKNISGSIEIVK |
| | ActRIIB-ECD (SEQ ID NO: | QGCWLDDFNCYDRQECVATEENPQVYFCCCEGNFCNERFTHLPEAGGPEVT |
| | 56) with 2 additional aa at N- | YEPPPTAPT |
| | terminus | |
| 1242 | N21Q mutant of Hybrid hu- | GEAETRECLFFNANWEKDRTQQTGVEPCYGDKDKRRHCFATWKNISGSIEIV |
| | ActRIIB-ECD (SEQ ID NO: | KQGCWLDDFNCYDRQECVATEENPQVYFCCCEGNFCNERFTHLPEAGGPEV |
| | 56) with 3 additional aa at N- | TYEPPPTAPT |
| | terminus | |
| 1243 | N22Q mutant of Hybrid hu- | RGEAETRECLFFNANWEKDRTQQTGVEPCYGDKDKRRHCFATWKNISGSIEI |
| | ActRIIB-ECD (SEQ ID NO: | VKQGCWLDDFNCYDRQECVATEENPQVYFCCCEGNFCNERFTHLPEAGGPE |
| | 56) with 4 additional aa at N- | VTYEPPPTAPT |
| | terminus | |
| 1244 | N23Q mutant of Hybrid hu- | GRGEAETRECLFFNANWEKDRTQQTGVEPCYGDKDKRRHCFATWKNISGSI |
| | ActRIIB-ECD (SEQ ID NO: | EIVKQGCWLDDFNCYDRQECVATEENPQVYFCCCEGNFCNERFTHLPEAGG |
| | 56) with 5 additional aa at N- | PEVTYEPPPTAPT |
| | terminus | |
| 1245 | N24Q mutant of Hybrid hu- | SGRGEAETRECLFFNANWEKDRTQQTGVEPCYGDKDKRRHCFATWKNISGS |
| | ActRIIB-ECD (SEQ ID NO: | IEIVKQGCWLDDFNCYDRQECVATEENPQVYFCCCEGNFCNERFTHLPEAG |
| | 56) with 6 additional aa at N- | GPEVTYEPPPTAPT |
| | terminus | |
| 1246 | N19Q mutant of Hybrid hu- | AETRECLFFNANWEKDRTQQTGVEPCYGDKDKRRHCFATWKNISGSIEIVK |
| | ActRIIB-ECD (SEQ ID NO: | QGCWLDDINCYDRQECVATEENPQVYFCCCEGNFCNERFTHLPEAGGPEVT |
| | 57) with 1 additional aa at N- | YEPPPTAPT |
| | terminus | |
| 1247 | N20Q mutant of Hybrid hu- | EAETRECLFFNANWEKDRTQQTGVEPCYGDKDKRRHCFATWKNISGSIEIVK |
| | ActRIIB-ECD (SEQ ID NO: | QGCWLDDINCYDRQECVATEENPQVYFCCCEGNFCNERFTHLPEAGGPEVT |
| | 57) with 2 additional aa at N- | YEPPPTAPT |
| | terminus | |
| 1248 | N21Q mutant of Hybrid hu- | GEAETRECLFFNANWEKDRTQQTGVEPCYGDKDKRRHCFATWKNISGSIEIV |
| | ActRIIB-ECD (SEQ ID NO: | KQGCWLDDINCYDRQECVATEENPQVYFCCCEGNFCNERFTHLPEAGGPEV |
| | 57) with 3 additional aa at N- | TYEPPPTAPT |
| | terminus | |
| 1249 | N22Q mutant of Hybrid hu- | RGEAETRECLFFNANWEKDRTQQTGVEPCYGDKDKRRHCFATWKNISGSIEI |
| | ActRIIB-ECD (SEQ ID NO: | VKQGCWLDDINCYDRQECVATEENPQVYFCCCEGNFCNERFTHLPEAGGPE |
| | 57) with 4 additional aa at N- | VTYEPPPTAPT |
| | terminus | |
| 1250 | N23Q mutant of Hybrid hu- | GRGEAETRECLFFNANWEKDRTQQTGVEPCYGDKDKRRHCFATWKNISGSI |
| | ActRIIB-ECD (SEQ ID NO: | EIVKQGCWLDDINCYDRQECVATEENPQVYFCCCEGNFCNERFTHLPEAGG |
| | 57) with 5 additional aa at N- | PEVTYEPPPTAPT |
| | terminus | |
| 1251 | N24Q mutant of Hybrid hu- | SGRGEAETRECLFFNANWEKDRTQQTGVEPCYGDKDKRRHCFATWKNISGS |
| | ActRIIB-ECD (SEQ ID NO: | IEIVKQGCWLDDINCYDRQECVATEENPQVYFCCCEGNFCNERFTHLPEAGG |
| | 57) with 6 additional aa at N- | PEVTYEPPPTAPT |
| | terminus | |
| 1252 | N19Q mutant of Hybrid hu- | AETRECLFFNANWEKDRTQQTGVEPCYGDKDKRRHCFATWKNISGSIEIVK |
| | ActRIIB-ECD (SEQ ID NO: | QGCWLDDINCYDRTDCVEKKDSPEVYFCCCEGNFCNERFTHLPEAGGPEVT |
| | 58) with 1 additional aa at N- | YEPPPTAPT |
| | terminus | |
| 1253 | N20Q mutant of Hybrid hu- | EAETRECLFFNANWEKDRTQQTGVEPCYGDKDKRRHCFATWKNISGSIEIVK |
| | ActRIIB-ECD (SEQ ID NO: | QGCWLDDINCYDRTDCVEKKDSPEVYFCCCEGNFCNERFTHLPEAGGPEVT |
| | 58) with 2 additional aa at N- | YEPPPTAPT |
| | terminus | |
| 1254 | N21Q mutant of Hybrid hu- | GEAETRECLFFNANWEKDRTQQTGVEPCYGDKDKRRHCFATWKNISGSIEIV |
| | ActRIIB-ECD (SEQ ID NO: | KQGCWLDDINCYDRTDCVEKKDSPEVYFCCCEGNFCNERFTHLPEAGGPEV |
| | 58) with 3 additional aa at N- | TYEPPPTAPT |
| | terminus | |
| 1255 | N22Q mutant of Hybrid hu- | RGEAETRECLFFNANWEKDRTQQTGVEPCYGDKDKRRHCFATWKNISGSIEI |
| | ActRIIB-ECD (SEQ ID NO: | VKQGCWLDDINCYDRTDCVEKKDSPEVYFCCCEGNFCNERFTHLPEAGGPE |
| | 58) with 4 additional aa at N- | VTYEPPPTAPT |
| | terminus | |
| 1256 | N23Q mutant of Hybrid hu- | GRGEAETRECLFFNANWEKDRTQQTGVEPCYGDKDKRRHCFATWKNISGSI |
| | ActRIIB-ECD (SEQ ID NO: | EIVKQGCWLDDINCYDRTDCVEKKDSPEVYFCCCEGNFCNERFTHLPEAGGP |
| | 58) with 5 additional aa at N- | EVTYEPPPTAPT |
| | terminus | |
| 1257 | N24Q mutant of Hybrid hu- | SGRGEAETRECLFFNANWEKDRTQQTGVEPCYGDKDKRRHCFATWKNISGS |
| | ActRIIB-ECD (SEQ ID NO: | IEIVKQGCWLDDINCYDRTDCVEKKDSPEVYFCCCEGNFCNERFTHLPEAGG |
| | 58) with 6 additional aa at N- | PEVTYEPPPTAPT |
| | terminus | |
| 1258 | N19Q mutant of Hybrid hu- | AETQECIYYNANWELERTQQSGLERCYGDKDKRRHCFATWKNISGSIEIVKQ |
| | ActRIIB-ECD (SEQ ID NO: | GCWLDDINCYDRQECVATEENPQVYFCCCEGNFCNERFTHLPEAGGPEVTY |
| | 59) with 1 additional aa at N- | EPPPTAPT |
| | terminus | |
| 1259 | N20Q mutant of Hybrid hu- | EAETQECIYYNANWELERTQQSGLERCYGDKDKRRHCFATWKNISGSIEIVK |
| | ActRIIB-ECD (SEQ ID NO: | QGCWLDDINCYDRQECVATEENPQVYFCCCEGNFCNERFTHLPEAGGPEVT |
| | 59) with 2 additional aa at N- | YEPPPTAPT |
| | terminus | |
| 1260 | N21Q mutant of Hybrid hu- | GEAETQECIYYNANWELERTQQSGLERCYGDKDKRRHCFATWKNISGSIEIV |
| | ActRIIB-ECD (SEQ ID NO: | KQGCWLDDINCYDRQECVATEENPQVYFCCCEGNFCNERFTHLPEAGGPEV |
| | 59) with 3 additional aa at N- | TYEPPPTAPT |
| | terminus | |
| 1261 | N22Q mutant of Hybrid hu- | RGEAETQECIYYNANWELERTQQSGLERCYGDKDKRRHCFATWKNISGSIEI |
| | ActRIIB-ECD (SEQ ID NO: | VKQGCWLDDINCYDRQECVATEENPQVYFCCCEGNFCNERFTHLPEAGGPE |
| | 59) with 4 additional aa at N- | VTYEPPPTAPT |
| | terminus | |
| 1262 | N23Q mutant of Hybrid hu- | GRGEAETQECIYYNANWELERTQQSGLERCYGDKDKRRHCFATWKNISGSI |
| | ActRIIB-ECD (SEQ ID NO: | EIVKQGCWLDDINCYDRQECVATEENPQVYFCCCEGNFCNERFTHLPEAGG |
| | 59) with 5 additional aa at N- | PEVTYEPPPTAPT |
| | terminus | |
| 1263 | N24Q mutant of Hybrid hu- | SGRGEAETQECIYYNANWELERTQQSGLERCYGDKDKRRHCFATWKNISGS |
| | ActRIIB-ECD (SEQ ID NO: | IEIVKQGCWLDDINCYDRQECVATEENPQVYFCCCEGNFCNERFTHLPEAGG |
| | 59) with 6 additional aa at N- | PEVTYEPPPTAPT |
| | terminus | |
| 1264 | N19Q mutant of Hybrid hu- | AETRECIYYNANWELERTQQSGLERCYGDKDKRRHCFATWKNISGSIEIVKQ |
| | ActRIIB-ECD (SEQ ID NO: | GCWLDDINCYDRTDCVEKKDSPEVYFCCCEGNFCNERFTHLPEAGGPEVTY |
| | 60) with 1 additional aa at N- | EPPPTAPT |
| | terminus | |
| 1265 | N20Q mutant of Hybrid hu- | EAETRECIYYNANWELERTQQSGLERCYGDKDKRRHCFATWKNISGSIEIVK |
| | ActRIIB-ECD (SEQ ID NO: | QGCWLDDINCYDRTDCVEKKDSPEVYFCCCEGNFCNERFTHLPEAGGPEVT |
| | 60) with 2 additional aa at N- | YEPPPTAPT |
| | terminus | |
| 1266 | N21Q mutant of Hybrid hu- | GEAETRECIYYNANWELERTQQSGLERCYGDKDKRRHCFATWKNISGSIEIV |
| | ActRIIB-ECD (SEQ ID NO: | KQGCWLDDINCYDRTDCVEKKDSPEVYFCCCEGNFCNERFTHLPEAGGPEV |
| | 60) with 3 additional aa at N- | TYEPPPTAPT |
| | terminus | |
| 1267 | N22Q mutant of Hybrid hu- | RGEAETRECIYYNANWELERTQQSGLERCYGDKDKRRHCFATWKNISGSIEI |
| | ActRIIB-ECD (SEQ ID NO: | VKQGCWLDDINCYDRTDCVEKKDSPEVYFCCCEGNFCNERFTHLPEAGGPE |
| | 60) with 4 additional aa at N- | VTYEPPPTAPT |
| | terminus | |
| 1268 | N23Q mutant of Hybrid hu- | GRGEAETRECIYYNANWELERTQQSGLERCYGDKDKRRHCFATWKNISGSI |
| | ActRIIB-ECD (SEQ ID NO: | EIVKQGCWLDDINCYDRTDCVEKKDSPEVYFCCCEGNFCNERFTHLPEAGGP |
| | 60) with 5 additional aa at N- | EVTYEPPPTAPT |
| | terminus | |
| 1269 | N24Q mutant of Hybrid hu- | SGRGEAETRECIYYNANWELERTQQSGLERCYGDKDKRRHCFATWKNISGS |
| | ActRIIB-ECD (SEQ ID NO: | IEIVKQGCWLDDINCYDRTDCVEKKDSPEVYFCCCEGNFCNERFTHLPEAGG |
| | 60) with 6 additional aa at N- | PEVTYEPPPTAPT |
| | terminus | |
| 1270 | N19Q mutant of Hybrid hu- | AETRECIYYNANWELERTQQSGLERCYGDKDKRRHCFATWKNISGSIEIVKQ |
| | ActRIIB-ECD (SEQ ID NO: | GCWLDDINCYDRTDCVEKKDSPEVYFCCCEGNMCNERFTHLPEAGGPEVTY |
| | 61) with 1 additional aa at N- | EPPPTAPT |
| | terminus | |
| 1271 | N20Q mutant of Hybrid hu- | EAETRECIYYNANWELERTQQSGLERCYGDKDKRRHCFATWKNISGSIEIVK |
| | ActRIIB-ECD (SEQ ID NO: | QGCWLDDINCYDRTDCVEKKDSPEVYFCCCEGNMCNERFTHLPEAGGPEVT |
| | 61) with 2 additional aa at N- | YEPPPTAPT |
| | terminus | |
| 1272 | N21Q mutant of Hybrid hu- | GEAETRECIYYNANWELERTQQSGLERCYGDKDKRRHCFATWKNISGSIEIV |
| | ActRIIB-ECD (SEQ ID NO: | KQGCWLDDINCYDRTDCVEKKDSPEVYFCCCEGNMCNERFTHLPEAGGPEV |
| | 61) with 3 additional aa at N- | TYEPPPTAPT |
| | terminus | |
| 1273 | N22Q mutant of Hybrid hu- | RGEAETRECIYYNANWELERTQQSGLERCYGDKDKRRHCFATWKNISGSIEI |
| | ActRIIB-ECD (SEQ ID NO: | VKQGCWLDDINCYDRTDCVEKKDSPEVYFCCCEGNMCNERFTHLPEAGGPE |
| | 61) with 4 additional aa at N- | VTYEPPPTAPT |
| | terminus | |
| 1274 | N23Q mutant of Hybrid hu- | GRGEAETRECIYYNANWELERTQQSGLERCYGDKDKRRHCFATWKNISGSI |
| | ActRIIB-ECD (SEQ ID NO: | EIVKQGCWLDDINCYDRTDCVEKKDSPEVYFCCCEGNMCNERFTHLPEAGG |
| | 61) with 5 additional aa at N- | PEVTYEPPPTAPT |
| | terminus | |
| 1275 | N24Q mutant of Hybrid hu- | SGRGEAETRECIYYNANWELERTQQSGLERCYGDKDKRRHCFATWKNISGS |
| | ActRIIB-ECD (SEQ ID NO: | IEIVKQGCWLDDINCYDRTDCVEKKDSPEVYFCCCEGNMCNERFTHLPEAG |
| | 61) with 6 additional aa at N- | GPEVTYEPPPTAPT |
| | terminus | |
| 1276 | N19Q mutant of Hybrid hu- | AETRECIYYNANWELERTQQSGLERCEGEQDKRLHCFATWKNISGSIEIVKQ |
| | ActRIIB-ECD (SEQ ID NO: | GCWLDDINCYDRTDCVEKKDSPEVYFCCCEGNFCNERFTHLPEAGGPEVTY |
| | 62) with 1 additional aa at N- | EPPPTAPT |
| | terminus | |
| 1277 | N20Q mutant of Hybrid hu- | EAETRECIYYNANWELERTQQSGLERCEGEQDKRLHCFATWKNISGSIEIVK |
| | ActRIIB-ECD (SEQ ID NO: | QGCWLDDINCYDRTDCVEKKDSPEVYFCCCEGNFCNERFTHLPEAGGPEVT |
| | 62) with 2 additional aa at N- | YEPPPTAPT |
| | terminus | |
| 1278 | N21Q mutant of Hybrid hu- | GEAETRECIYYNANWELERTQQSGLERCEGEQDKRLHCFATWKNISGSIEIV |
| | ActRIIB-ECD (SEQ ID NO: | KQGCWLDDINCYDRTDCVEKKDSPEVYFCCCEGNFCNERFTHLPEAGGPEV |
| | 62) with 3 additional aa at N- | TYEPPPTAPT |
| | terminus | |
| 1279 | N22Q mutant of Hybrid hu- | RGEAETRECIYYNANWELERTQQSGLERCEGEQDKRLHCFATWKNISGSIEI |
| | ActRIIB-ECD (SEQ ID NO: | VKQGCWLDDINCYDRTDCVEKKDSPEVYFCCCEGNFCNERFTHLPEAGGPE |
| | 62) with 4 additional aa at N- | VTYEPPPTAPT |
| | terminus | |
| 1280 | N23Q mutant of Hybrid hu- | GRGEAETRECIYYNANWELERTQQSGLERCEGEQDKRLHCFATWKNISGSIE |
| | ActRIIB-ECD (SEQ ID NO: | IVKQGCWLDDINCYDRTDCVEKKDSPEVYFCCCEGNFCNERFTHLPEAGGPE |
| | 62) with 5 additional aa at N- | VTYEPPPTAPT |
| | terminus | |
| 1281 | N24Q mutant of Hybrid hu- | SGRGEAETRECIYYNANWELERTQQSGLERCEGEQDKRLHCFATWKNISGSI |
| | ActRIIB-ECD (SEQ ID NO: | EIVKQGCWLDDINCYDRTDCVEKKDSPEVYFCCCEGNFCNERFTHLPEAGGP |
| | 62) with 6 additional aa at N- | EVTYEPPPTAPT |
| | terminus | |
| 1282 | N19Q mutant of Hybrid hu- | AETRECIYYNANWELERTQQSGLERCEGEQDKRLHCFATWKNISGSIEIVKQ |
| | ActRIIB-ECD (SEQ ID NO: | GCWLDDINCYDRTDCVATEENPQVYFCCCEGNFCNERFTHLPEAGGPEVTY |
| | 63) with 1 additional aa at N- | EPPPTAPT |
| | terminus | |
| 1283 | N20Q mutant of Hybrid hu- | EAETRECIYYNANWELERTQQSGLERCEGEQDKRLHCFATWKNISGSIEIVK |
| | ActRIIB-ECD (SEQ ID NO: | QGCWLDDINCYDRTDCVATEENPQVYFCCCEGNFCNERFTHLPEAGGPEVT |
| | 63) with 2 additional aa at N- | YEPPPTAPT |
| | terminus | |
| 1284 | N21Q mutant of Hybrid hu- | GEAETRECIYYNANWELERTQQSGLERCEGEQDKRLHCFATWKNISGSIEIV |
| | ActRIIB-ECD (SEQ ID NO: | KQGCWLDDINCYDRTDCVATEENPQVYFCCCEGNFCNERFTHLPEAGGPEV |
| | 63) with 3 additional aa at N- | TYEPPPTAPT |
| | terminus | |
| 1285 | N22Q mutant of Hybrid hu- | RGEAETRECIYYNANWELERTQQSGLERCEGEQDKRLHCFATWKNISGSIEI |
| | ActRIIB-ECD (SEQ ID NO: | VKQGCWLDDINCYDRTDCVATEENPQVYFCCCEGNFCNERFTHLPEAGGPE |
| | 63) with 4 additional aa at N- | VTYEPPPTAPT |
| | terminus | |
| 1286 | N23Q mutant of Hybrid hu- | GRGEAETRECIYYNANWELERTQQSGLERCEGEQDKRLHCFATWKNISGSIE |
| | ActRIIB-ECD (SEQ ID NO: | IVKQGCWLDDINCYDRTDCVATEENPQVYFCCCEGNFCNERFTHLPEAGGP |
| | 63) with 5 additional aa at N- | EVTYEPPPTAPT |
| | terminus | |
| 1287 | N24Q mutant of Hybrid hu- | SGRGEAETRECIYYNANWELERTQQSGLERCEGEQDKRLHCFATWKNISGSI |
| | ActRIIB-ECD (SEQ ID NO: | EIVKQGCWLDDINCYDRTDCVATEENPQVYFCCCEGNFCNERFTHLPEAGG |
| | 63) with 6 additional aa at N- | PEVTYEPPPTAPT |
| | terminus | |
| 1288 | N19Q mutant of Hybrid hu- | AETRECIYYNANWELERTQQSGLERCEGEQDKRLHCFATWKNISGSIEIVKQ |
| | ActRIIB-ECD (SEQ ID NO: | GCWLDDFNCYDRTDCVATEENPQVYFCCCEGNFCNERFTHLPEAGGPEVTY |
| | 64) with 1 additional aa at N- | EPPPTAPT |
| | terminus | |
| 1289 | N20Q mutant of Hybrid hu- | EAETRECIYYNANWELERTQQSGLERCEGEQDKRLHCFATWKNISGSIEIVK |
| | ActRIIB-ECD (SEQ ID NO: | QGCWLDDFNCYDRTDCVATEENPQVYFCCCEGNFCNERFTHLPEAGGPEVT |
| | 64) with 2 additional aa at N- | YEPPPTAPT |
| | terminus | |
| 1290 | N21Q mutant of Hybrid hu- | GEAETRECIYYNANWELERTQQSGLERCEGEQDKRLHCFATWKNISGSIEIV |
| | ActRIIB-ECD (SEQ ID NO: | KQGCWLDDFNCYDRTDCVATEENPQVYFCCCEGNFCNERFTHLPEAGGPEV |
| | 64) with 3 additional aa at N- | TYEPPPTAPT |
| | terminus | |
| 1291 | N22Q mutant of Hybrid hu- | RGEAETRECIYYNANWELERTQQSGLERCEGEQDKRLHCFATWKNISGSIEI |
| | ActRIIB-ECD (SEQ ID NO: | VKQGCWLDDFNCYDRTDCVATEENPQVYFCCCEGNFCNERFTHLPEAGGPE |
| | 64) with 4 additional aa at N- | VTYEPPPTAPT |
| | terminus | |
| 1292 | N23Q mutant of Hybrid hu- | GRGEAETRECIYYNANWELERTQQSGLERCEGEQDKRLHCFATWKNISGSIE |
| | ActRIIB-ECD (SEQ ID NO: | IVKQGCWLDDFNCYDRTDCVATEENPQVYFCCCEGNFCNERFTHLPEAGGP |
| | 64) with 5 additional aa at N- | EVTYEPPPTAPT |
| | terminus | |
| 1293 | N24Q mutant of Hybrid hu- | SGRGEAETRECIYYNANWELERTQQSGLERCEGEQDKRLHCFATWKNISGSI |
| | ActRIIB-ECD (SEQ ID NO: | EIVKQGCWLDDFNCYDRTDCVATEENPQVYFCCCEGNFCNERFTHLPEAGG |
| | 64) with 6 additional aa at N- | PEVTYEPPPTAPT |
| | terminus | |
| 1294 | N19Q mutant of Hybrid hu- | AETRECIYYNANWELERTQQSGLERCEGEQDKRLHCFATWKNISGSIEIVKQ |
| | ActRIIB-ECD (SEQ ID NO: | GCWLDDFNCYDRQECVATEENPQVYFCCCEGNFCNERFTHLPEAGGPEVTY |
| | 65) with 1 additional aa at N- | EPPPTAPT |
| | terminus | |
| 1295 | N20Q mutant of Hybrid hu- | EAETRECIYYNANWELERTQQSGLERCEGEQDKRLHCFATWKNISGSIEIVK |
| | ActRIIB-ECD (SEQ ID NO: | QGCWLDDFNCYDRQECVATEENPQVYFCCCEGNFCNERFTHLPEAGGPEVT |
| | 65) with 2 additional aa at N- | YEPPPTAPT |
| | terminus | |
| 1296 | N21Q mutant of Hybrid hu- | GEAETRECIYYNANWELERTQQSGLERCEGEQDKRLHCFATWKNISGSIEIV |
| | ActRIIB-ECD (SEQ ID NO: | KQGCWLDDFNCYDRQECVATEENPQVYFCCCEGNFCNERFTHLPEAGGPEV |
| | 65) with 3 additional aa at N- | TYEPPPTAPT |
| | terminus | |
| 1297 | N22Q mutant of Hybrid hu- | RGEAETRECIYYNANWELERTQQSGLERCEGEQDKRLHCFATWKNISGSIEI |
| | ActRIIB-ECD (SEQ ID NO: | VKQGCWLDDFNCYDRQECVATEENPQVYFCCCEGNFCNERFTHLPEAGGPE |
| | 65) with 4 additional aa at N- | VTYEPPPTAPT |
| | terminus | |
| 1298 | N23Q mutant of Hybrid hu- | GRGEAETRECIYYNANWELERTQQSGLERCEGEQDKRLHCFATWKNISGSIE |
| | ActRIIB-ECD (SEQ ID NO: | IVKQGCWLDDFNCYDRQECVATEENPQVYFCCCEGNFCNERFTHLPEAGGP |
| | 65) with 5 additional aa at N- | EVTYEPPPTAPT |
| | terminus | |
| 1299 | N24Q mutant of Hybrid hu- | SGRGEAETRECIYYNANWELERTQQSGLERCEGEQDKRLHCFATWKNISGSI |
| | ActRIIB-ECD (SEQ ID NO: | EIVKQGCWLDDFNCYDRQECVATEENPQVYFCCCEGNFCNERFTHLPEAGG |
| | 65) with 6 additional aa at N- | PEVTYEPPPTAPT |
| | terminus | |
| 1300 | N19Q mutant of Hybrid hu- | AETQECIYYNANWELERTQQSGLERCYGDKDKRRHCYASWRNSSGTIELVK |
| | ActRIIB-ECD (SEQ ID NO: | KGCWLDDINCYDRTDCVEKKDSPEVYFCCCEGNFCNERFTHLPEAGGPEVT |
| | 66) with 1 additional aa at N- | YEPPPTAPT |
| | terminus | |
| 1301 | N20Q mutant of Hybrid hu- | EAETQECIYYNANWELERTQQSGLERCYGDKDKRRHCYASWRNSSGTIELV |
| | ActRIIB-ECD (SEQ ID NO: | KKGCWLDDINCYDRTDCVEKKDSPEVYFCCCEGNFCNERFTHLPEAGGPEV |
| | 66) with 2 additional aa at N- | TYEPPPTAPT |
| | terminus | |
| 1302 | N21Q mutant of Hybrid hu- | GEAETQECIYYNANWELERTQQSGLERCYGDKDKRRHCYASWRNSSGTIEL |
| | ActRIIB-ECD (SEQ ID NO: | VKKGCWLDDINCYDRTDCVEKKDSPEVYFCCCEGNFCNERFTHLPEAGGPE |
| | 66) with 3 additional aa at N- | VTYEPPPTAPT |
| | terminus | |
| 1303 | N22Q mutant of Hybrid hu- | RGEAETQECIYYNANWELERTQQSGLERCYGDKDKRRHCYASWRNSSGTIE |
| | ActRIIB-ECD (SEQ ID NO: | LVKKGCWLDDINCYDRTDCVEKKDSPEVYFCCCEGNFCNERFTHLPEAGGP |
| | 66) with 4 additional aa at N- | EVTYEPPPTAPT |
| | terminus | |
| 1304 | N23Q mutant of Hybrid hu- | GRGEAETQECIYYNANWELERTQQSGLERCYGDKDKRRHCYASWRNSSGTI |
| | ActRIIB-ECD (SEQ ID NO: | ELVKKGCWLDDINCYDRTDCVEKKDSPEVYFCCCEGNFCNERFTHLPEAGG |
| | 66) with 5 additional aa at N- | PEVTYEPPPTAPT |
| | terminus | |
| 1305 | N24Q mutant of Hybrid hu- | SGRGEAETQECIYYNANWELERTQQSGLERCYGDKDKRRHCYASWRNSSG |
| | ActRIIB-ECD (SEQ ID NO: | TIELVKKGCWLDDINCYDRTDCVEKKDSPEVYFCCCEGNFCNERFTHLPEAG |
| | 66) with 6 additional aa at N- | GPEVTYEPPPTAPT |
| | terminus | |
| 1306 | N19Q mutant of Hybrid hu- | AETQECIYYNANWELERTQQSGLERCYGDKDKRRHCYASWRNSSGTIELVK |
| | ActRIIB-ECD (SEQ ID NO: | KGCWLDDINCYDRTDCVEKKDSPEVYFCCCEGNMCNERFTHLPEAGGPEVT |
| | 67) with 1 additional aa at N- | YEPPPTAPT |
| | terminus | |
| 1307 | N20Q mutant of Hybrid hu- | EAETQECIYYNANWELERTQQSGLERCYGDKDKRRHCYASWRNSSGTIELV |
| | ActRIIB-ECD (SEQ ID NO: | KKGCWLDDINCYDRTDCVEKKDSPEVYFCCCEGNMCNERFTHLPEAGGPEV |
| | 67) with 2 additional aa at N- | TYEPPPTAPT |
| | terminus | |
| 1308 | N21Q mutant of Hybrid hu- | GEAETQECIYYNANWELERTQQSGLERCYGDKDKRRHCYASWRNSSGTIEL |
| | ActRIIB-ECD (SEQ ID NO: | VKKGCWLDDINCYDRTDCVEKKDSPEVYFCCCEGNMCNERFTHLPEAGGPE |
| | 67) with 3 additional aa at N- | VTYEPPPTAPT |
| | terminus | |
| 1309 | N22Q mutant of Hybrid hu- | RGEAETQECIYYNANWELERTQQSGLERCYGDKDKRRHCYASWRNSSGTIE |
| | ActRIIB-ECD (SEQ ID NO: | LVKKGCWLDDINCYDRTDCVEKKDSPEVYFCCCEGNMCNERFTHLPEAGGP |
| | 67) with 4 additional aa at N- | EVTYEPPPTAPT |
| | terminus | |
| 1310 | N23Q mutant of Hybrid hu- | GRGEAETQECIYYNANWELERTQQSGLERCYGDKDKRRHCYASWRNSSGTI |
| | ActRIIB-ECD (SEQ ID NO: | ELVKKGCWLDDINCYDRTDCVEKKDSPEVYFCCCEGNMCNERFTHLPEAG |
| | 67) with 5 additional aa at N- | GPEVTYEPPPTAPT |
| | terminus | |
| 1311 | N24Q mutant of Hybrid hu- | SGRGEAETQECIYYNANWELERTQQSGLERCYGDKDKRRHCYASWRNSSG |
| | ActRIIB-ECD (SEQ ID NO: | TIELVKKGCWLDDINCYDRTDCVEKKDSPEVYFCCCEGNMCNERFTHLPEA |
| | 67) with 6 additional aa at N- | GGPEVTYEPPPTAPT |
| | terminus | |
| 1312 | N19Q mutant of Hybrid hu- | AETQECIYYNANWELERTQQSGLERCYGDKDKRRHCFATWKNISGSIEIVKQ |
| | ActRIIB-ECD (SEQ ID NO: | GCWLDDINCYDRTDCVEKKDSPEVYFCCCEGNMCNERFTHLPEAGGPEVTY |
| | 68) with 1 additional aa at N- | EPPPTAPT |
| | terminus | |
| 1313 | N20Q mutant of Hybrid hu- | EAETQECIYYNANWELERTQQSGLERCYGDKDKRRHCFATWKNISGSIEIVK |
| | ActRIIB-ECD (SEQ ID NO: | QGCWLDDINCYDRTDCVEKKDSPEVYFCCCEGNMCNERFTHLPEAGGPEVT |
| | 68) with 2 additional aa at N- | YEPPPTAPT |
| | terminus | |
| 1314 | N21Q mutant of Hybrid hu- | GEAETQECIYYNANWELERTQQSGLERCYGDKDKRRHCFATWKNISGSIEIV |
| | ActRIIB-ECD (SEQ ID NO: | KQGCWLDDINCYDRTDCVEKKDSPEVYFCCCEGNMCNERFTHLPEAGGPEV |
| | 68) with 3 additional aa at N- | TYEPPPTAPT |
| | terminus | |
| 1315 | N22Q mutant of Hybrid hu- | RGEAETQECIYYNANWELERTQQSGLERCYGDKDKRRHCFATWKNISGSIEI |
| | ActRIIB-ECD (SEQ ID NO: | VKQGCWLDDINCYDRTDCVEKKDSPEVYFCCCEGNMCNERFTHLPEAGGPE |
| | 68) with 4 additional aa at N- | VTYEPPPTAPT |
| | terminus | |
| 1316 | N23Q mutant of Hybrid hu- | GRGEAETQECIYYNANWELERTQQSGLERCYGDKDKRRHCFATWKNISGSI |
| | ActRIIB-ECD (SEQ ID NO: | EIVKQGCWLDDINCYDRTDCVEKKDSPEVYFCCCEGNMCNERFTHLPEAGG |
| | 68) with 5 additional aa at N- | PEVTYEPPPTAPT |
| | terminus | |
| 1317 | N24Q mutant of Hybrid hu- | SGRGEAETQECIYYNANWELERTQQSGLERCYGDKDKRRHCFATWKNISGS |
| | ActRIIB-ECD (SEQ ID NO: | IEIVKQGCWLDDINCYDRTDCVEKKDSPEVYFCCCEGNMCNERFTHLPEAG |
| | 68) with 6 additional aa at N- | GPEVTYEPPPTAPT |
| | terminus | |
| 1318 | N19Q mutant of Hybrid hu- | AETQECIYYNANWELERTQQSGLERCYGDKDKRRHCFATWKNISGSIEIVKQ |
| | ActRIIB-ECD (SEQ ID NO: | GCWLDDINCYDRTDCVEKKDSPEVYFCCCEGNFCNERFTHLPEAGGPEVTY |
| | 69) with 1 additional aa at N- | EPPPTAPT |
| | terminus | |
| 1319 | N20Q mutant of Hybrid hu- | EAETQECIYYNANWELERTQQSGLERCYGDKDKRRHCFATWKNISGSIEIVK |
| | ActRIIB-ECD (SEQ ID NO: | QGCWLDDINCYDRTDCVEKKDSPEVYFCCCEGNFCNERFTHLPEAGGPEVT |
| | 69) with 2 additional aa at N- | YEPPPTAPT |
| | terminus | |
| 1320 | N21Q mutant of Hybrid hu- | GEAETQECIYYNANWELERTQQSGLERCYGDKDKRRHCFATWKNISGSIEIV |
| | ActRIIB-ECD (SEQ ID NO: | KQGCWLDDINCYDRTDCVEKKDSPEVYFCCCEGNFCNERFTHLPEAGGPEV |
| | 69) with 3 additional aa at N- | TYEPPPTAPT |
| | terminus | |
| 1321 | N22Q mutant of Hybrid hu- | RGEAETQECIYYNANWELERTQQSGLERCYGDKDKRRHCFATWKNISGSIEI |
| | ActRIIB-ECD (SEQ ID NO: | VKQGCWLDDINCYDRTDCVEKKDSPEVYFCCCEGNFCNERFTHLPEAGGPE |
| | 69) with 4 additional aa at N- | VTYEPPPTAPT |
| | terminus | |
| 1322 | N23Q mutant of Hybrid hu- | GRGEAETQECIYYNANWELERTQQSGLERCYGDKDKRRHCFATWKNISGSI |
| | ActRIIB-ECD (SEQ ID NO: | EIVKQGCWLDDINCYDRTDCVEKKDSPEVYFCCCEGNFCNERFTHLPEAGGP |
| | 69) with 5 additional aa at N- | EVTYEPPPTAPT |
| | terminus | |
| 1323 | N24Q mutant of Hybrid hu- | SGRGEAETQECIYYNANWELERTQQSGLERCYGDKDKRRHCFATWKNISGS |
| | ActRIIB-ECD (SEQ ID NO: | IEIVKQGCWLDDINCYDRTDCVEKKDSPEVYFCCCEGNFCNERFTHLPEAGG |
| | 69) with 6 additional aa at N- | PEVTYEPPPTAPT |
| | terminus | |
| 1324 | N19Q mutant of Hybrid hu- | AETRECLFFNANWEKDRTQQTGVEPCEGEQDKRLHCFATWKNISGSIEIVKQ |
| | ActRIIB-ECD (SEQ ID NO: | GCWLDDINCYDRTDCVEKKDSPEVYFCCCEGNFCNERFTHLPEAGGPEVTY |
| | 70) with 1 additional aa at N- | EPPPTAPT |
| | terminus | |
| 1325 | N20Q mutant of Hybrid hu- | EAETRECLFFNANWEKDRTQQTGVEPCEGEQDKRLHCFATWKNISGSIEIVK |
| | ActRIIB-ECD (SEQ ID NO: | QGCWLDDINCYDRTDCVEKKDSPEVYFCCCEGNFCNERFTHLPEAGGPEVT |
| | 70) with 2 additional aa at N- | YEPPPTAPT |
| | terminus | |
| 1326 | N21Q mutant of Hybrid hu- | GEAETRECLFFNANWEKDRTQQTGVEPCEGEQDKRLHCFATWKNISGSIEIV |
| | ActRIIB-ECD (SEQ ID NO: | KQGCWLDDINCYDRTDCVEKKDSPEVYFCCCEGNFCNERFTHLPEAGGPEV |
| | 70) with 3 additional aa at N- | TYEPPPTAPT |
| | terminus | |
| 1327 | N22Q mutant of Hybrid hu- | RGEAETRECLFFNANWEKDRTQQTGVEPCEGEQDKRLHCFATWKNISGSIEI |
| | ActRIIB-ECD (SEQ ID NO: | VKQGCWLDDINCYDRTDCVEKKDSPEVYFCCCEGNFCNERFTHLPEAGGPE |
| | 70) with 4 additional aa at N- | VTYEPPPTAPT |
| | terminus | |
| 1328 | N23Q mutant of Hybrid hu- | GRGEAETRECLFFNANWEKDRTQQTGVEPCEGEQDKRLHCFATWKNISGSI |
| | ActRIIB-ECD (SEQ ID NO: | EIVKQGCWLDDINCYDRTDCVEKKDSPEVYFCCCEGNFCNERFTHLPEAGGP |
| | 70) with 5 additional aa at N- | EVTYEPPPTAPT |
| | terminus | |
| 1329 | N24Q mutant of Hybrid hu- | SGRGEAETRECLFFNANWEKDRTQQTGVEPCEGEQDKRLHCFATWKNISGSI |
| | ActRIIB-ECD (SEQ ID NO: | EIVKQGCWLDDINCYDRTDCVEKKDSPEVYFCCCEGNFCNERFTHLPEAGGP |
| | 70) with 6 additional aa at N- | EVTYEPPPTAPT |
| | terminus | |
| 1330 | N19Q mutant of Hybrid hu- | AETRECLFFNANWEKDRTQQSGVEPCEGEQDKRLHCYASWRNSSGTIELVK |
| | ActRIIB-ECD (SEQ ID NO: | KGCWLDDINCYDRTDCVEKKDSPEVYFCCCEGNFCNERFTHLPEAGGPEVT |
| | 71) with 1 additional aa at N- | YEPPPTAPT |
| | terminus | |
| 1331 | N20Q mutant of Hybrid hu- | EAETRECLFFNANWEKDRTQQSGVEPCEGEQDKRLHCYASWRNSSGTIELV |
| | ActRIIB-ECD (SEQ ID NO: | KKGCWLDDINCYDRTDCVEKKDSPEVYFCCCEGNFCNERFTHLPEAGGPEV |
| | 71) with 2 additional aa at N- | TYEPPPTAPT |
| | terminus | |
| 1332 | N21Q mutant of Hybrid hu- | GEAETRECLFFNANWEKDRTQQSGVEPCEGEQDKRLHCYASWRNSSGTIEL |
| | ActRIIB-ECD (SEQ ID NO: | VKKGCWLDDINCYDRTDCVEKKDSPEVYFCCCEGNFCNERFTHLPEAGGPE |
| | 71) with 3 additional aa at N- | VTYEPPPTAPT |
| | terminus | |
| 1333 | N22Q mutant of Hybrid hu- | RGEAETRECLFFNANWEKDRTQQSGVEPCEGEQDKRLHCYASWRNSSGTIE |
| | ActRIIB-ECD (SEQ ID NO: | LVKKGCWLDDINCYDRTDCVEKKDSPEVYFCCCEGNFCNERFTHLPEAGGP |
| | 71) with 4 additional aa at N- | EVTYEPPPTAPT |
| | terminus | |
| 1334 | N23Q mutant of Hybrid hu- | GRGEAETRECLFFNANWEKDRTQQSGVEPCEGEQDKRLHCYASWRNSSGTI |
| | ActRIIB-ECD (SEQ ID NO: | ELVKKGCWLDDINCYDRTDCVEKKDSPEVYFCCCEGNFCNERFTHLPEAGG |
| | 71) with 5 additional aa at N- | PEVTYEPPPTAPT |
| | terminus | |
| 1335 | N24Q mutant of Hybrid hu- | SGRGEAETRECLFFNANWEKDRTQQSGVEPCEGEQDKRLHCYASWRNSSGT |
| | ActRIIB-ECD (SEQ ID NO: | IELVKKGCWLDDINCYDRTDCVEKKDSPEVYFCCCEGNFCNERFTHLPEAG |
| | 71) with 6 additional aa at N- | GPEVTYEPPPTAPT |
| | terminus | |
| 1336 | N19Q mutant of Hybrid hu- | AETRECLFFNANWEKDRTQQSGVEPCYGDKDKRRHCYASWRNSSGTIELVK |
| | ActRIIB-ECD (SEQ ID NO: | KGCWLDDINCYDRTDCVEKKDSPEVYFCCCEGNFCNERFTHLPEAGGPEVT |
| | 72) with 1 additional aa at N- | YEPPPTAPT |
| | terminus | |
| 1337 | N20Q mutant of Hybrid hu- | EAETRECLFFNANWEKDRTQQSGVEPCYGDKDKRRHCYASWRNSSGTIELV |
| | ActRIIB-ECD (SEQ ID NO: | KKGCWLDDINCYDRTDCVEKKDSPEVYFCCCEGNFCNERFTHLPEAGGPEV |
| | 72) with 2 additional aa at N- | TYEPPPTAPT |
| | terminus | |
| 1338 | N21Q mutant of Hybrid hu- | GEAETRECLFFNANWEKDRTQQSGVEPCYGDKDKRRHCYASWRNSSGTIEL |
| | ActRIIB-ECD (SEQ ID NO: | VKKGCWLDDINCYDRTDCVEKKDSPEVYFCCCEGNFCNERFTHLPEAGGPE |
| | 72) with 3 additional aa at N- | VTYEPPPTAPT |
| | terminus | |
| 1339 | N22Q mutant of Hybrid hu- | RGEAETRECLFFNANWEKDRTQQSGVEPCYGDKDKRRHCYASWRNSSGTIE |
| | ActRIIB-ECD (SEQ ID NO: | LVKKGCWLDDINCYDRTDCVEKKDSPEVYFCCCEGNFCNERFTHLPEAGGP |
| | 72) with 4 additional aa at N- | EVTYEPPPTAPT |
| | terminus | |
| 1340 | N23Q mutant of Hybrid hu- | GRGEAETRECLFFNANWEKDRTQQSGVEPCYGDKDKRRHCYASWRNSSGTI |
| | ActRIIB-ECD (SEQ ID NO: | ELVKKGCWLDDINCYDRTDCVEKKDSPEVYFCCCEGNFCNERFTHLPEAGG |
| | 72) with 5 additional aa at N- | PEVTYEPPPTAPT |
| | terminus | |
| 1341 | N24Q mutant of Hybrid hu- | SGRGEAETRECLFFNANWEKDRTQQSGVEPCYGDKDKRRHCYASWRNSSG |
| | ActRIIB-ECD (SEQ ID NO: | TIELVKKGCWLDDINCYDRTDCVEKKDSPEVYFCCCEGNFCNERFTHLPEAG |
| | 72) with 6 additional aa at N- | GPEVTYEPPPTAPT |
| | terminus | |
| 1342 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDFNCYDRTDCVATEENPQVYFCCCEGNFCNERFTHLPEAGGPEVT |
| | 73) with 1 additional aa at N- | YEPPPTAPT |
| | terminus | |
| 1343 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDFNCYDRTDCVATEENPQVYFCCCEGNFCNERFTHLPEAGGPEV |
| | 73) with 2 additional aa at N- | TYEPPPTAPT |
| | terminus | |
| 1344 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDFNCYDRTDCVATEENPQVYFCCCEGNFCNERFTHLPEAGGPE |
| | 73) with 3 additional aa at N- | VTYEPPPTAPT |
| | terminus | |
| 1345 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDFNCYDRTDCVATEENPQVYFCCCEGNFCNERFTHLPEAGGP |
| | 73) with 4 additional aa at N- | EVTYEPPPTAPT |
| | terminus | |
| 1346 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDFNCYDRTDCVATEENPQVYFCCCEGNFCNERFTHLPEAGG |
| | 73) with 5 additional aa at N- | PEVTYEPPPTAPT |
| | terminus | |
| 1347 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDFNCYDRTDCVATEENPQVYFCCCEGNFCNERFTHLPEA |
| | 73) with 6 additional aa at N- | GGPEVTYEPPPTAPT |
| | terminus | |
| 1348 | N19Q mutant of Hybrid hu- | AETRECIYYNANWELERTQQSGLERCYGDKDKRRHCYASWRNSSGTIELVK |
| | ActRIIB-ECD (SEQ ID NO: | QGCWLDDFNCYDRTDCVATEENPQVYFCCCEGNFCNERFTHLPEAGGPEVT |
| | 74) with 1 additional aa at N- | YEPPPTAPT |
| | terminus | |
| 1349 | N20Q mutant of Hybrid hu- | EAETRECIYYNANWELERTQQSGLERCYGDKDKRRHCYASWRNSSGTIELV |
| | ActRIIB-ECD (SEQ ID NO: | KQGCWLDDFNCYDRTDCVATEENPQVYFCCCEGNFCNERFTHLPEAGGPEV |
| | 74) with 2 additional aa at N- | TYEPPPTAPT |
| | terminus | |
| 1350 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKQGCWLDDFNCYDRTDCVATEENPQVYFCCCEGNFCNERFTHLPEAGGPE |
| | 74) with 3 additional aa at N- | VTYEPPPTAPT |
| | terminus | |
| 1351 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKQGCWLDDFNCYDRTDCVATEENPQVYFCCCEGNFCNERFTHLPEAGGP |
| | 74) with 4 additional aa at N- | EVTYEPPPTAPT |
| | terminus | |
| 1352 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKQGCWLDDFNCYDRTDCVATEENPQVYFCCCEGNFCNERFTHLPEAGG |
| | 74) with 5 additional aa at N- | PEVTYEPPPTAPT |
| | terminus | |
| 1353 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKQGCWLDDFNCYDRTDCVATEENPQVYFCCCEGNFCNERFTHLPEA |
| | 74) with 6 additional aa at N- | GGPEVTYEPPPTAPT |
| | terminus | |
| 1354 | N19Q mutant of Hybrid hu- | AETRECIYYNANWELERTQQSGLERCYGDKDKRRHCYASWRNSSGTIEIVK |
| | ActRIIB-ECD (SEQ ID NO: | KGCWLDDFNCYDRTDCVATEENPQVYFCCCEGNFCNERFTHLPEAGGPEVT |
| | 75) with 1 additional aa at N- | YEPPPTAPT |
| | terminus | |
| 1355 | N20Q mutant of Hybrid hu- | EAETRECIYYNANWELERTQQSGLERCYGDKDKRRHCYASWRNSSGTIEIV |
| | ActRIIB-ECD (SEQ ID NO: | KKGCWLDDFNCYDRTDCVATEENPQVYFCCCEGNFCNERFTHLPEAGGPEV |
| | 75) with 2 additional aa at N- | TYEPPPTAPT |
| | terminus | |
| 1356 | N21Q mutant of Hybrid hu- | GEAETRECIYYNANWELERTQQSGLERCYGDKDKRRHCYASWRNSSGTIEI |
| | ActRIIB-ECD (SEQ ID NO: | VKKGCWLDDFNCYDRTDCVATEENPQVYFCCCEGNFCNERFTHLPEAGGPE |
| | 75) with 3 additional aa at N- | VTYEPPPTAPT |
| | terminus | |
| 1357 | N22Q mutant of Hybrid hu- | RGEAETRECIYYNANWELERTQQSGLERCYGDKDKRRHCYASWRNSSGTIE |
| | ActRIIB-ECD (SEQ ID NO: | IVKKGCWLDDFNCYDRTDCVATEENPQVYFCCCEGNFCNERFTHLPEAGGP |
| | 75) with 4 additional aa at N- | EVTYEPPPTAPT |
| | terminus | |
| 1358 | N23Q mutant of Hybrid hu- | GRGEAETRECIYYNANWELERTQQSGLERCYGDKDKRRHCYASWRNSSGTI |
| | ActRIIB-ECD (SEQ ID NO: | EIVKKGCWLDDFNCYDRTDCVATEENPQVYFCCCEGNFCNERFTHLPEAGG |
| | 75) with 5 additional aa at N- | PEVTYEPPPTAPT |
| | terminus | |
| 1359 | N24Q mutant of Hybrid hu- | SGRGEAETRECIYYNANWELERTQQSGLERCYGDKDKRRHCYASWRNSSG |
| | ActRIIB-ECD (SEQ ID NO: | TIEIVKKGCWLDDFNCYDRTDCVATEENPQVYFCCCEGNFCNERFTHLPEAG |
| | 75) with 6 additional aa at N- | GPEVTYEPPPTAPT |
| | terminus | |
| 1360 | N19Q mutant of Hybrid hu- | AETRECIYYNANWELERTQQSGLERCYGDKDKRRHCYASWRNSSGSIELVK |
| | ActRIIB-ECD (SEQ ID NO: | KGCWLDDFNCYDRTDCVATEENPQVYFCCCEGNFCNERFTHLPEAGGPEVT |
| | 76) with 1 additional aa at N- | YEPPPTAPT |
| | terminus | |
| 1361 | N20Q mutant of Hybrid hu- | EAETRECIYYNANWELERTQQSGLERCYGDKDKRRHCYASWRNSSGSIELV |
| | ActRIIB-ECD (SEQ ID NO: | KKGCWLDDFNCYDRTDCVATEENPQVYFCCCEGNFCNERFTHLPEAGGPEV |
| | 76) with 2 additional aa at N- | TYEPPPTAPT |
| | terminus | |
| 1362 | N21Q mutant of Hybrid hu- | GEAETRECIYYNANWELERTQQSGLERCYGDKDKRRHCYASWRNSSGSIEL |
| | ActRIIB-ECD (SEQ ID NO: | VKKGCWLDDFNCYDRTDCVATEENPQVYFCCCEGNFCNERFTHLPEAGGPE |
| | 76) with 3 additional aa at N- | VTYEPPPTAPT |
| | terminus | |
| 1363 | N22Q mutant of Hybrid hu- | RGEAETRECIYYNANWELERTQQSGLERCYGDKDKRRHCYASWRNSSGSIE |
| | ActRIIB-ECD (SEQ ID NO: | LVKKGCWLDDFNCYDRTDCVATEENPQVYFCCCEGNFCNERFTHLPEAGGP |
| | 76) with 4 additional aa at N- | EVTYEPPPTAPT |
| | terminus | |
| 1364 | N23Q mutant of Hybrid hu- | GRGEAETRECIYYNANWELERTQQSGLERCYGDKDKRRHCYASWRNSSGSI |
| | ActRIIB-ECD (SEQ ID NO: | ELVKKGCWLDDFNCYDRTDCVATEENPQVYFCCCEGNFCNERFTHLPEAGG |
| | 76) with 5 additional aa at N- | PEVTYEPPPTAPT |
| | terminus | |
| 1365 | N24Q mutant of Hybrid hu- | SGRGEAETRECIYYNANWELERTQQSGLERCYGDKDKRRHCYASWRNSSGS |
| | ActRIIB-ECD (SEQ ID NO: | IELVKKGCWLDDFNCYDRTDCVATEENPQVYFCCCEGNFCNERFTHLPEAG |
| | 76) with 6 additional aa at N- | GPEVTYEPPPTAPT |
| | terminus | |
| 1366 | N19Q mutant of Hybrid hu- | AETRECIYYNANWELERTQQSGLERCYGDKDKRRHCYASWRNSSGSIEIVK |
| | ActRIIB-ECD (SEQ ID NO: | KGCWLDDFNCYDRTDCVATEENPQVYFCCCEGNFCNERFTHLPEAGGPEVT |
| | 77) with 1 additional aa at N- | YEPPPTAPT |
| | terminus | |
| 1367 | N20Q mutant of Hybrid hu- | EAETRECIYYNANWELERTQQSGLERCYGDKDKRRHCYASWRNSSGSIEIV |
| | ActRIIB-ECD (SEQ ID NO: | KKGCWLDDFNCYDRTDCVATEENPQVYFCCCEGNFCNERFTHLPEAGGPEV |
| | 77) with 2 additional aa at N- | TYEPPPTAPT |
| | terminus | |
| 1368 | N21Q mutant of Hybrid hu- | GEAETRECIYYNANWELERTQQSGLERCYGDKDKRRHCYASWRNSSGSIEI |
| | ActRIIB-ECD (SEQ ID NO: | VKKGCWLDDFNCYDRTDCVATEENPQVYFCCCEGNFCNERFTHLPEAGGPE |
| | 77) with 3 additional aa at N- | VTYEPPPTAPT |
| | terminus | |
| 1369 | N22Q mutant of Hybrid hu- | RGEAETRECIYYNANWELERTQQSGLERCYGDKDKRRHCYASWRNSSGSIEI |
| | ActRIIB-ECD (SEQ ID NO: | VKKGCWLDDFNCYDRTDCVATEENPQVYFCCCEGNFCNERFTHLPEAGGPE |
| | 77) with 4 additional aa at N- | VTYEPPPTAPT |
| | terminus | |
| 1370 | N23Q mutant of Hybrid hu- | GRGEAETRECIYYNANWELERTQQSGLERCYGDKDKRRHCYASWRNSSGSI |
| | ActRIIB-ECD (SEQ ID NO: | EIVKKGCWLDDFNCYDRTDCVATEENPQVYFCCCEGNFCNERFTHLPEAGG |
| | 77) with 5 additional aa at N- | PEVTYEPPPTAPT |
| | terminus | |
| 1371 | N24Q mutant of Hybrid hu- | SGRGEAETRECIYYNANWELERTQQSGLERCYGDKDKRRHCYASWRNSSGS |
| | ActRIIB-ECD (SEQ ID NO: | IEIVKKGCWLDDFNCYDRTDCVATEENPQVYFCCCEGNFCNERFTHLPEAG |
| | 77) with 6 additional aa at N- | GPEVTYEPPPTAPT |
| | terminus | |
| 1372 | N19Q mutant of Hybrid hu- | AETRECIYYNANWELERTQQSGLERCYGDKDKRRHCYASWRNSSGSIEIVK |
| | ActRIIB-ECD (SEQ ID NO: | QGCWLDDFNCYDRTDCVATEENPQVYFCCCEGNFCNERFTHLPEAGGPEVT |
| | 78) with 1 additional aa at N- | YEPPPTAPT |
| | terminus | |
| 1373 | N20Q mutant of Hybrid hu- | EAETRECIYYNANWELERTQQSGLERCYGDKDKRRHCYASWRNSSGSIEIV |
| | ActRIIB-ECD (SEQ ID NO: | KQGCWLDDFNCYDRTDCVATEENPQVYFCCCEGNFCNERFTHLPEAGGPEV |
| | 78) with 2 additional aa at N- | TYEPPPTAPT |
| | terminus | |
| 1374 | N21Q mutant of Hybrid hu- | GEAETRECIYYNANWELERTQQSGLERCYGDKDKRRHCYASWRNSSGSIEI |
| | ActRIIB-ECD (SEQ ID NO: | VKQGCWLDDFNCYDRTDCVATEENPQVYFCCCEGNFCNERFTHLPEAGGPE |
| | 78) with 3 additional aa at N- | VTYEPPPTAPT |
| | terminus | |
| 1375 | N22Q mutant of Hybrid hu- | RGEAETRECIYYNANWELERTQQSGLERCYGDKDKRRHCYASWRNSSGSIEI |
| | ActRIIB-ECD (SEQ ID NO: | VKQGCWLDDFNCYDRTDCVATEENPQVYFCCCEGNFCNERFTHLPEAGGPE |
| | 78) with 4 additional aa at N- | VTYEPPPTAPT |
| | terminus | |
| 1376 | N23Q mutant of Hybrid hu- | GRGEAETRECIYYNANWELERTQQSGLERCYGDKDKRRHCYASWRNSSGSI |
| | ActRIIB-ECD (SEQ ID NO: | EIVKQGCWLDDFNCYDRTDCVATEENPQVYFCCCEGNFCNERFTHLPEAGG |
| | 78) with 5 additional aa at N- | PEVTYEPPPTAPT |
| | terminus | |
| 1377 | N24Q mutant of Hybrid hu- | SGRGEAETRECIYYNANWELERTQQSGLERCYGDKDKRRHCYASWRNSSGS |
| | ActRIIB-ECD (SEQ ID NO: | IEIVKQGCWLDDFNCYDRTDCVATEENPQVYFCCCEGNFCNERFTHLPEAG |
| | 78) with 6 additional aa at N- | GPEVTYEPPPTAPT |
| | terminus | |
| 1378 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRTDCVATEENPQVYFCCCEGNFCNERFTHLPEAGGPEVT |
| | 79) with 1 additional aa at N- | YEPPPTAPT |
| | terminus | |
| 1379 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRTDCVATEENPQVYFCCCEGNFCNERFTHLPEAGGPEV |
| | 79) with 2 additional aa at N- | TYEPPPTAPT |
| | terminus | |
| 1380 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRTDCVATEENPQVYFCCCEGNFCNERFTHLPEAGGPE |
| | 79) with 3 additional aa at N- | VTYEPPPTAPT |
| | terminus | |
| 1381 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRTDCVATEENPQVYFCCCEGNFCNERFTHLPEAGGP |
| | 79) with 4 additional aa at N- | EVTYEPPPTAPT |
| | terminus | |
| 1382 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRTDCVATEENPQVYFCCCEGNFCNERFTHLPEAGG |
| | 79) with 5 additional aa at N- | PEVTYEPPPTAPT |
| | terminus | |
| 1383 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRTDCVATEENPQVYFCCCEGNFCNERFTHLPEAG |
| | 79) with 6 additional aa at N- | GPEVTYEPPPTAPT |
| | terminus | |
| 1384 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATEENPQVYFCCCEGNFCNEKFSYFPEMEVTQPT |
| | 80) with 1 additional aa at N- | SNPVTPKPP |
| | terminus | |
| 1385 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATEENPQVYFCCCEGNFCNEKFSYFPEMEVTQP |
| | 80) with 2 additional aa at N- | TSNPVTPKPP |
| | terminus | |
| 1386 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATEENPQVYFCCCEGNFCNEKFSYFPEMEVT |
| | 80) with 3 additional aa at N- | QPTSNPVTPKPP |
| | terminus | |
| 1387 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATEENPQVYFCCCEGNFCNEKFSYFPEMEVT |
| | 80) with 4 additional aa at N- | QPTSNPVTPKPP |
| | terminus | |
| 1388 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATEENPQVYFCCCEGNFCNEKFSYFPEMEV |
| | 80) with 5 additional aa at N- | TQPTSNPVTPKPP |
| | terminus | |
| 1389 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATEENPQVYFCCCEGNFCNEKFSYFPEME |
| | 80) with 6 additional aa at N- | VTQPTSNPVTPKPP |
| | terminus | |
| 1390 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATEENPQVYFCCCEGNFCNEKFSYFPQMEVTQPT |
| | 81) with 1 additional aa at N- | SNPVTPKPP |
| | terminus | |
| 1391 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATEENPQVYFCCCEGNFCNEKFSYFPQMEVTQ |
| | 81) with 2 additional aa at N- | PTSNPVTPKPP |
| | terminus | |
| 1392 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATEENPQVYFCCCEGNFCNEKFSYFPQMEVT |
| | 81) with 3 additional aa at N- | QPTSNPVTPKPP |
| | terminus | |
| 1393 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATEENPQVYFCCCEGNFCNEKFSYFPQMEV |
| | 81) with 4 additional aa at N- | TQPTSNPVTPKPP |
| | terminus | |
| 1394 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATEENPQVYFCCCEGNFCNEKFSYFPQME |
| | 81) with 5 additional aa at N- | VTQPTSNPVTPKPP |
| | terminus | |
| 1395 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATEENPQVYFCCCEGNFCNEKFSYFPQME |
| | 81) with 6 additional aa at N- | VTQPTSNPVTPKPP |
| | terminus | |
| 1396 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDINCYDRTDCVEKKDSPEVYFCCCEGNFCNEKFSYFPEMEVTQPT |
| | 82) with 1 additional aa at N- | SNPVTPKPP |
| | terminus | |
| 1397 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDINCYDRTDCVEKKDSPEVYFCCCEGNFCNEKFSYFPEMEVTQP |
| | 82) with 2 additional aa at N- | TSNPVTPKPP |
| | terminus | |
| 1398 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDINCYDRTDCVEKKDSPEVYFCCCEGNFCNEKFSYFPEMEVTQ |
| | 82) with 3 additional aa at N- | PTSNPVTPKPP |
| | terminus | |
| 1399 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDINCYDRTDCVEKKDSPEVYFCCCEGNFCNEKFSYFPEMEVT |
| | 82) with 4 additional aa at N- | QPTSNPVTPKPP |
| | terminus | |
| 1400 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDINCYDRTDCVEKKDSPEVYFCCCEGNFCNEKFSYFPEMEV |
| | 82) with 5 additional aa at N- | TQPTSNPVTPKPP |
| | terminus | |
| 1401 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDINCYDRTDCVEKKDSPEVYFCCCEGNFCNEKFSYFPEME |
| | 82) with 6 additional aa at N- | VTQPTSNPVTPKPP |
| | terminus | |
| 1402 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDINCYDRTDCVEKKDSPEVYFCCCEGNFCNEKFSYFPQMEVTQPT |
| | 83) with 1 additional aa at N- | SNPVTPKPP |
| | terminus | |
| 1403 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDINCYDRTDCVEKKDSPEVYFCCCEGNFCNEKFSYFPQMEVTQP |
| | 83) with 2 additional aa at N- | TSNPVTPKPP |
| | terminus | |
| 1404 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDINCYDRTDCVEKKDSPEVYFCCCEGNFCNEKFSYFPQMEVTQ |
| | 83) with 3 additional aa at N- | PTSNPVTPKPP |
| | terminus | |
| 1405 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDINCYDRTDCVEKKDSPEVYFCCCEGNFCNEKFSYFPQMEVT |
| | 83) with 4 additional aa at N- | QPTSNPVTPKPP |
| | terminus | |
| 1406 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDINCYDRTDCVEKKDSPEVYFCCCEGNFCNEKFSYFPQMEV |
| | 83) with 5 additional aa at N- | TQPTSNPVTPKPP |
| | terminus | |
| 1407 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDINCYDRTDCVEKKDSPEVYFCCCEGNFCNEKFSYFPQM |
| | 83) with 6 additional aa at N- | EVTQPTSNPVTPKPP |
| | terminus | |
| 1408 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDFNCYDRQECVATEENPQVYFCCCEGNFCNEKFSYFPQMEVTQPT |
| | 84) with 1 additional aa at N- | SNPVTPKPP |
| | terminus | |
| 1409 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDFNCYDRQECVATEENPQVYFCCCEGNFCNEKFSYFPQMEVTQ |
| | 84) with 2 additional aa at N- | PTSNPVTPKPP |
| | terminus | |
| 1410 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDFNCYDRQECVATEENPQVYFCCCEGNFCNEKFSYFPQMEVT |
| | 84) with 3 additional aa at N- | QPTSNPVTPKPP |
| | terminus | |
| 1411 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDFNCYDRQECVATEENPQVYFCCCEGNFCNEKFSYFPQMEV |
| | 84) with 4 additional aa at N- | TQPTSNPVTPKPP |
| | terminus | |
| 1412 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDFNCYDRQECVATEENPQVYFCCCEGNFCNEKFSYFPQME |
| | 84) with 5 additional aa at N- | VTQPTSNPVTPKPP |
| | terminus | |
| 1413 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDFNCYDRQECVATEENPQVYFCCCEGNFCNEKFSYFPQM |
| | 84) with 6 additional aa at N- | EVTQPTSNPVTPKPP |
| | terminus | |
| 1414 | N19Q mutant of Hybrid hu- | AETRECIYYNANWELERTQQSGLERCYGDKDKRRHCYASWRNSSGTIELVK |
| | ActRIIB-ECD (SEQ ID NO: | QGCWLDDFNCYDRQECVATEENPQVYFCCCEGNFCNEKFSYFPQMEVTQPT |
| | 85) with 1 additional aa at N- | SNPVTPKPP |
| | terminus | |
| 1415 | N20Q mutant of Hybrid hu- | EAETRECIYYNANWELERTQQSGLERCYGDKDKRRHCYASWRNSSGTIELV |
| | ActRIIB-ECD (SEQ ID NO: | KQGCWLDDFNCYDRQECVATEENPQVYFCCCEGNFCNEKFSYFPQMEVTQ |
| | 85) with 2 additional aa at N- | PTSNPVTPKPP |
| | terminus | |
| 1416 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKQGCWLDDFNCYDRQECVATEENPQVYFCCCEGNFCNEKFSYFPQMEVT |
| | 85) with 3 additional aa at N- | QPTSNPVTPKPP |
| | terminus | |
| 1417 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKQGCWLDDFNCYDRQECVATEENPQVYFCCCEGNFCNEKFSYFPQMEV |
| | 85) with 4 additional aa at N- | TQPTSNPVTPKPP |
| | terminus | |
| 1418 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKQGCWLDDFNCYDRQECVATEENPQVYFCCCEGNFCNEKFSYFPQME |
| | 85) with 5 additional aa at N- | VTQPTSNPVTPKPP |
| | terminus | |
| 1419 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKQGCWLDDFNCYDRQECVATEENPQVYFCCCEGNFCNEKFSYFPQM |
| | 85) with 6 additional aa at N- | EVTQPTSNPVTPKPP |
| | terminus | |
| 1420 | N19Q mutant of Hybrid hu- | AETRECIYYNANWELERTQQSGLERCYGDKDKRRHCYASWRNSSGTIEIVK |
| | ActRIIB-ECD (SEQ ID NO: | KGCWLDDFNCYDRQECVATKENPQVYFCCCEGNFCNEKFSYFPQMEVTQP |
| | 86) with 1 additional aa at N- | TSNPVTPKPP |
| | terminus | |
| 1421 | N20Q mutant of Hybrid hu- | EAETRECIYYNANWELERTQQSGLERCYGDKDKRRHCYASWRNSSGTIEIV |
| | ActRIIB-ECD (SEQ ID NO: | KKGCWLDDFNCYDRQECVATKENPQVYFCCCEGNFCNEKFSYFPQMEVTQ |
| | 86) with 2 additional aa at N- | PTSNPVTPKPP |
| | terminus | |
| 1422 | N21Q mutant of Hybrid hu- | GEAETRECIYYNANWELERTQQSGLERCYGDKDKRRHCYASWRNSSGTIEI |
| | ActRIIB-ECD (SEQ ID NO: | VKKGCWLDDFNCYDRQECVATKENPQVYFCCCEGNFCNEKFSYFPQMEVT |
| | 86) with 3 additional aa at N- | QPTSNPVTPKPP |
| | terminus | |
| 1423 | N22Q mutant of Hybrid hu- | RGEAETRECIYYNANWELERTQQSGLERCYGDKDKRRHCYASWRNSSGTIE |
| | ActRIIB-ECD (SEQ ID NO: | IVKKGCWLDDFNCYDRQECVATKENPQVYFCCCEGNFCNEKFSYFPQMEVT |
| | 86) with 4 additional aa at N- | QPTSNPVTPKPP |
| | terminus | |
| 1424 | N23Q mutant of Hybrid hu- | GRGEAETRECIYYNANWELERTQQSGLERCYGDKDKRRHCYASWRNSSGTI |
| | ActRIIB-ECD (SEQ ID NO: | EIVKKGCWLDDFNCYDRQECVATKENPQVYFCCCEGNFCNEKFSYFPQMEV |
| | 86) with 5 additional aa at N- | TQPTSNPVTPKPP |
| | terminus | |
| 1425 | N24Q mutant of Hybrid hu- | SGRGEAETRECIYYNANWELERTQQSGLERCYGDKDKRRHCYASWRNSSG |
| | ActRIIB-ECD (SEQ ID NO: | TIEIVKKGCWLDDFNCYDRQECVATKENPQVYFCCCEGNFCNEKFSYFPQM |
| | 86) with 6 additional aa at N- | EVTQPTSNPVTPKPP |
| | terminus | |
| 1426 | N19Q mutant of Hybrid hu- | AETRECIYYNANWELERTQQSGLERCYGDKDKRRHCYASWRNSSGSIELVK |
| | ActRIIB-ECD (SEQ ID NO: | KGCWLDDFNCYDRQECVATKENPQVYFCCCEGNFCNEKFSYFPQMEVTQP |
| | 87) with 1 additional aa at N- | TSNPVTPKPP |
| | terminus | |
| 1427 | N20Q mutant of Hybrid hu- | EAETRECIYYNANWELERTQQSGLERCYGDKDKRRHCYASWRNSSGSIELV |
| | ActRIIB-ECD (SEQ ID NO: | KKGCWLDDFNCYDRQECVATKENPQVYFCCCEGNFCNEKFSYFPQMEVTQ |
| | 87) with 2 additional aa at N- | PTSNPVTPKPP |
| | terminus | |
| 1428 | N21Q mutant of Hybrid hu- | GEAETRECIYYNANWELERTQQSGLERCYGDKDKRRHCYASWRNSSGSIEL |
| | ActRIIB-ECD (SEQ ID NO: | VKKGCWLDDFNCYDRQECVATKENPQVYFCCCEGNFCNEKFSYFPQMEVT |
| | 87) with 3 additional aa at N- | QPTSNPVTPKPP |
| | terminus | |
| 1429 | N22Q mutant of Hybrid hu- | RGEAETRECIYYNANWELERTQQSGLERCYGDKDKRRHCYASWRNSSGSIE |
| | ActRIIB-ECD (SEQ ID NO: | LVKKGCWLDDFNCYDRQECVATKENPQVYFCCCEGNFCNEKFSYFPQMEV |
| | 87) with 4 additional aa at N- | TQPTSNPVTPKPP |
| | terminus | |
| 1430 | N23Q mutant of Hybrid hu- | GRGEAETRECIYYNANWELERTQQSGLERCYGDKDKRRHCYASWRNSSGSI |
| | ActRIIB-ECD (SEQ ID NO: | ELVKKGCWLDDFNCYDRQECVATKENPQVYFCCCEGNFCNEKFSYFPQME |
| | 87) with 5 additional aa at N- | VTQPTSNPVTPKPP |
| | terminus | |
| 1431 | N24Q mutant of Hybrid hu- | SGRGEAETRECIYYNANWELERTQQSGLERCYGDKDKRRHCYASWRNSSGS |
| | ActRIIB-ECD (SEQ ID NO: | IELVKKGCWLDDFNCYDRQECVATKENPQVYFCCCEGNFCNEKFSYFPQME |
| | 87) with 6 additional aa at N- | VTQPTSNPVTPKPP |
| | terminus | |
| 1432 | N19Q mutant of Hybrid hu- | AETRECIYYNANWELERTQQSGLERCEGEQDKRLHCYASWRNSSGSIELVK |
| | ActRIIB-ECD (SEQ ID NO: | KGCWLDDFNCYDRQECVATEENPQVYFCCCEGNFCNEKFSYFPQMEVTQPT |
| | 88) with 1 additional aa at N- | SNPVTPKPP |
| | terminus | |
| 1433 | N20Q mutant of Hybrid hu- | EAETRECIYYNANWELERTQQSGLERCEGEQDKRLHCYASWRNSSGSIELV |
| | ActRIIB-ECD (SEQ ID NO: | KKGCWLDDFNCYDRQECVATEENPQVYFCCCEGNFCNEKFSYFPQMEVTQ |
| | 88) with 2 additional aa at N- | PTSNPVTPKPP |
| | terminus | |
| 1434 | N21Q mutant of Hybrid hu- | GEAETRECIYYNANWELERTQQSGLERCEGEQDKRLHCYASWRNSSGSIEL |
| | ActRIIB-ECD (SEQ ID NO: | VKKGCWLDDFNCYDRQECVATEENPQVYFCCCEGNFCNEKFSYFPQMEVT |
| | 88) with 3 additional aa at N- | QPTSNPVTPKPP |
| | terminus | |
| 1435 | N22Q mutant of Hybrid hu- | RGEAETRECIYYNANWELERTQQSGLERCEGEQDKRLHCYASWRNSSGSIEL |
| | ActRIIB-ECD (SEQ ID NO: | VKKGCWLDDFNCYDRQECVATEENPQVYFCCCEGNFCNEKFSYFPQMEVT |
| | 88) with 4 additional aa at N- | QPTSNPVTPKPP |
| | terminus | |
| 1436 | N23Q mutant of Hybrid hu- | GRGEAETRECIYYNANWELERTQQSGLERCEGEQDKRLHCYASWRNSSGSI |
| | ActRIIB-ECD (SEQ ID NO: | ELVKKGCWLDDFNCYDRQECVATEENPQVYFCCCEGNFCNEKFSYFPQME |
| | 88) with 5 additional aa at N- | VTQPTSNPVTPKPP |
| | terminus | |
| 1437 | N24Q mutant of Hybrid hu- | SGRGEAETRECIYYNANWELERTQQSGLERCEGEQDKRLHCYASWRNSSGS |
| | ActRIIB-ECD (SEQ ID NO: | IELVKKGCWLDDFNCYDRQECVATEENPQVYFCCCEGNFCNEKFSYFPQME |
| | 88) with 6 additional aa at N- | VTQPTSNPVTPKPP |
| | terminus | |
| 1438 | N19Q mutant of Hybrid hu- | AETRECIYYNANWELERTQQSGLERCEGEQDKRLHCYASWRNSSGTIEIVKK |
| | ActRIIB-ECD (SEQ ID NO: | GCWLDDFNCYDRQECVATEENPQVYFCCCEGNFCNEKFSYFPQMEVTQPTS |
| | 89) with 1 additional aa at N- | NPVTPKPP |
| | terminus | |
| 1439 | N20Q mutant of Hybrid hu- | EAETRECIYYNANWELERTQQSGLERCEGEQDKRLHCYASWRNSSGTIEIVK |
| | ActRIIB-ECD (SEQ ID NO: | KGCWLDDFNCYDRQECVATEENPQVYFCCCEGNFCNEKFSYFPQMEVTQPT |
| | 89) with 2 additional aa at N- | SNPVTPKPP |
| | terminus | |
| 1440 | N21Q mutant of Hybrid hu- | GEAETRECIYYNANWELERTQQSGLERCEGEQDKRLHCYASWRNSSGTIEIV |
| | ActRIIB-ECD (SEQ ID NO: | KKGCWLDDFNCYDRQECVATEENPQVYFCCCEGNFCNEKFSYFPQMEVTQ |
| | 89) with 3 additional aa at N- | PTSNPVTPKPP |
| | terminus | |
| 1441 | N22Q mutant of Hybrid hu- | RGEAETRECIYYNANWELERTQQSGLERCEGEQDKRLHCYASWRNSSGTIEI |
| | ActRIIB-ECD (SEQ ID NO: | VKKGCWLDDFNCYDRQECVATEENPQVYFCCCEGNFCNEKFSYFPQMEVT |
| | 89) with 4 additional aa at N- | QPTSNPVTPKPP |
| | terminus | |
| 1442 | N23Q mutant of Hybrid hu- | GRGEAETRECIYYNANWELERTQQSGLERCEGEQDKRLHCYASWRNSSGTI |
| | ActRIIB-ECD (SEQ ID NO: | EIVKKGCWLDDFNCYDRQECVATEENPQVYFCCCEGNFCNEKFSYFPQMEV |
| | 89) with 5 additional aa at N- | TQPTSNPVTPKPP |
| | terminus | |
| 1443 | N24Q mutant of Hybrid hu- | SGRGEAETRECIYYNANWELERTQQSGLERCEGEQDKRLHCYASWRNSSGT |
| | ActRIIB-ECD (SEQ ID NO: | IEIVKKGCWLDDFNCYDRQECVATEENPQVYFCCCEGNFCNEKFSYFPQME |
| | 89) with 6 additional aa at N- | VTQPTSNPVTPKPP |
| | terminus | |
| 1444 | N19Q mutant of Hybrid hu- | AETRECIYYNANWELERTQQSGLERCEGEQDKRLHCYASWRNSSGTIELVK |
| | ActRIIB-ECD (SEQ ID NO: | QGCWLDDFNCYDRQECVATEENPQVYFCCCEGNFCNEKFSYFPQMEVTQPT |
| | 90) with 1 additional aa at N- | SNPVTPKPP |
| | terminus | |
| 1445 | N20Q mutant of Hybrid hu- | EAETRECIYYNANWELERTQQSGLERCEGEQDKRLHCYASWRNSSGTIELV |
| | ActRIIB-ECD (SEQ ID NO: | KQGCWLDDFNCYDRQECVATEENPQVYFCCCEGNFCNEKFSYFPQMEVTQ |
| | 90) with 2 additional aa at N- | PTSNPVTPKPP |
| | terminus | |
| 1446 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKQGCWLDDFNCYDRQECVATEENPQVYFCCCEGNFCNEKFSYFPQMEVT |
| | 90) with 3 additional aa at N- | QPTSNPVTPKPP |
| | terminus | |
| 1447 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKQGCWLDDFNCYDRQECVATEENPQVYFCCCEGNFCNEKFSYFPQMEV |
| | 90) with 4 additional aa at N- | TQPTSNPVTPKPP |
| | terminus | |
| 1448 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKQGCWLDDFNCYDRQECVATEENPQVYFCCCEGNFCNEKFSYFPQME |
| | 90) with 5 additional aa at N- | VTQPTSNPVTPKPP |
| | terminus | |
| 1449 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKQGCWLDDFNCYDRQECVATEENPQVYFCCCEGNFCNEKFSYFPQME |
| | 90) with 6 additional aa at N- | VTQPTSNPVTPKPP |
| | terminus | |
| 1450 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVETEENPQVYFCCCEGNFCNEKFSYFPQMEVTQPT |
| | 91) with 1 additional aa at N- | SNPVTPKPP |
| | terminus | |
| 1451 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVETEENPQVYFCCCEGNFCNEKFSYFPQMEVTQP |
| | 91) with 2 additional aa at N- | TSNPVTPKPP |
| | terminus | |
| 1452 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVETEENPQVYFCCCEGNFCNEKFSYFPQMEVT |
| | 91) with 3 additional aa at N- | QPTSNPVTPKPP |
| | terminus | |
| 1453 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVETEENPQVYFCCCEGNFCNEKFSYFPQMEVT |
| | 91) with 4 additional aa at N- | QPTSNPVTPKPP |
| | terminus | |
| 1454 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVETEENPQVYFCCCEGNFCNEKFSYFPQMEV |
| | 91) with 5 additional aa at N- | TQPTSNPVTPKPP |
| | terminus | |
| 1455 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVETEENPQVYFCCCEGNFCNEKFSYFPQME |
| | 91) with 6 additional aa at N- | VTQPTSNPVTPKPP |
| | terminus | |
| 1456 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVAKEENPQVYFCCCEGNFCNEKFSYFPQMEVTQP |
| | 92) with 1 additional aa at N- | TSNPVTPKPP |
| | terminus | |
| 1457 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVAKEENPQVYFCCCEGNFCNEKFSYFPQMEVTQ |
| | 92) with 2 additional aa at N- | PTSNPVTPKPP |
| | terminus | |
| 1458 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVAKEENPQVYFCCCEGNFCNEKFSYFPQMEVT |
| | 92) with 3 additional aa at N- | QPTSNPVTPKPP |
| | terminus | |
| 1459 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVAKEENPQVYFCCCEGNFCNEKFSYFPQMEV |
| | 92) with 4 additional aa at N- | TQPTSNPVTPKPP |
| | terminus | |
| 1460 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVAKEENPQVYFCCCEGNFCNEKFSYFPQME |
| | 92) with 5 additional aa at N- | VTQPTSNPVTPKPP |
| | terminus | |
| 1461 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVAKEENPQVYFCCCEGNFCNEKFSYFPQME |
| | 92) with 6 additional aa at N- | VTQPTSNPVTPKPP |
| | terminus | |
| 1462 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATKENPQVYFCCCEGNFCNEKFSYFPQMEVTQP |
| | 93) with 1 additional aa at N- | TSNPVTPKPP |
| | terminus | |
| 1463 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATKENPQVYFCCCEGNFCNEKFSYFPQMEVTQ |
| | 93) with 2 additional aa at N- | PTSNPVTPKPP |
| | terminus | |
| 1464 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATKENPQVYFCCCEGNFCNEKFSYFPQMEVT |
| | 93) with 3 additional aa at N- | QPTSNPVTPKPP |
| | terminus | |
| 1465 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATKENPQVYFCCCEGNFCNEKFSYFPQMEV |
| | 93) with 4 additional aa at N- | TQPTSNPVTPKPP |
| | terminus | |
| 1466 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATKENPQVYFCCCEGNFCNEKFSYFPQME |
| | 93) with 5 additional aa at N- | VTQPTSNPVTPKPP |
| | terminus | |
| 1467 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATKENPQVYFCCCEGNFCNEKFSYFPQME |
| | 93) with 6 additional aa at N- | VTQPTSNPVTPKPP |
| | terminus | |
| 1468 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATEDNPQVYFCCCEGNFCNEKFSYFPQMEVTQP |
| | 94) with 1 additional aa at N- | TSNPVTPKPP |
| | terminus | |
| 1469 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATEDNPQVYFCCCEGNFCNEKFSYFPQMEVTQ |
| | 94) with 2 additional aa at N- | PTSNPVTPKPP |
| | terminus | |
| 1470 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATEDNPQVYFCCCEGNFCNEKFSYFPQMEVT |
| | 94) with 3 additional aa at N- | QPTSNPVTPKPP |
| | terminus | |
| 1471 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATEDNPQVYFCCCEGNFCNEKFSYFPQMEV |
| | 94) with 4 additional aa at N- | TQPTSNPVTPKPP |
| | terminus | |
| 1472 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATEDNPQVYFCCCEGNFCNEKFSYFPQME |
| | 94) with 5 additional aa at N- | VTQPTSNPVTPKPP |
| | terminus | |
| 1473 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATEDNPQVYFCCCEGNFCNEKFSYFPQME |
| | 94) with 6 additional aa at N- | VTQPTSNPVTPKPP |
| | terminus | |
| 1474 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATEESPQVYFCCCEGNFCNEKFSYFPQMEVTQPT |
| | 95) with 1 additional aa at N- | SNPVTPKPP |
| | terminus | |
| 1475 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATEESPQVYFCCCEGNFCNEKFSYFPQMEVTQP |
| | 95) with 2 additional aa at N- | TSNPVTPKPP |
| | terminus | |
| 1476 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATEESPQVYFCCCEGNFCNEKFSYFPQMEVT |
| | 95) with 3 additional aa at N- | QPTSNPVTPKPP |
| | terminus | |
| 1477 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATEESPQVYFCCCEGNFCNEKFSYFPQMEVT |
| | 95) with 4 additional aa at N- | QPTSNPVTPKPP |
| | terminus | |
| 1478 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATEESPQVYFCCCEGNFCNEKFSYFPQMEV |
| | 95) with 5 additional aa at N- | TQPTSNPVTPKPP |
| | terminus | |
| 1479 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATEESPQVYFCCCEGNFCNEKFSYFPQME |
| | 95) with 6 additional aa at N- | VTQPTSNPVTPKPP |
| | terminus | |
| 1480 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATEENPEVYFCCCEGNFCNEKFSYFPQMEVTQPT |
| | 96) with 1 additional aa at N- | SNPVTPKPP |
| | terminus | |
| 1481 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATEENPEVYFCCCEGNFCNEKFSYFPQMEVTQP |
| | 96) with 2 additional aa at N- | TSNPVTPKPP |
| | terminus | |
| 1482 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATEENPEVYFCCCEGNFCNEKFSYFPQMEVT |
| | 96) with 3 additional aa at N- | QPTSNPVTPKPP |
| | terminus | |
| 1483 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATEENPEVYFCCCEGNFCNEKFSYFPQMEVT |
| | 96) with 4 additional aa at N- | QPTSNPVTPKPP |
| | terminus | |
| 1484 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATEENPEVYFCCCEGNFCNEKFSYFPQMEV |
| | 96) with 5 additional aa at N- | TQPTSNPVTPKPP |
| | terminus | |
| 1485 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATEENPEVYFCCCEGNFCNEKFSYFPQME |
| | 96) with 6 additional aa at N- | VTQPTSNPVTPKPP |
| | terminus | |
| 1486 | N19Q mutant of Hybrid hu- | AETRECIYYNANWELERTQQSGLERCEGDKDKRLHCYASWRNSSGTIELVK |
| | ActRIIB-ECD (SEQ ID NO: | KGCWLDDFNCYDRQECVETEENPQVYFCCCEGNFCNEKFSYFPQMEVTQPT |
| | 97) with 1 additional aa at N- | SNPVTPKPP |
| | terminus | |
| 1487 | N20Q mutant of Hybrid hu- | EAETRECIYYNANWELERTQQSGLERCEGDKDKRLHCYASWRNSSGTIELV |
| | ActRIIB-ECD (SEQ ID NO: | KKGCWLDDFNCYDRQECVETEENPQVYFCCCEGNFCNEKFSYFPQMEVTQP |
| | 97) with 2 additional aa at N- | TSNPVTPKPP |
| | terminus | |
| 1488 | N21Q mutant of Hybrid hu- | GEAETRECIYYNANWELERTQQSGLERCEGDKDKRLHCYASWRNSSGTIEL |
| | ActRIIB-ECD (SEQ ID NO: | VKKGCWLDDFNCYDRQECVETEENPQVYFCCCEGNFCNEKFSYFPQMEVT |
| | 97) with 3 additional aa at N- | QPTSNPVTPKPP |
| | terminus | |
| 1489 | N22Q mutant of Hybrid hu- | RGEAETRECIYYNANWELERTQQSGLERCEGDKDKRLHCYASWRNSSGTIE |
| | ActRIIB-ECD (SEQ ID NO: | LVKKGCWLDDFNCYDRQECVETEENPQVYFCCCEGNFCNEKFSYFPQMEVT |
| | 97) with 4 additional aa at N- | QPTSNPVTPKPP |
| | terminus | |
| 1490 | N23Q mutant of Hybrid hu- | GRGEAETRECIYYNANWELERTQQSGLERCEGDKDKRLHCYASWRNSSGTI |
| | ActRIIB-ECD (SEQ ID NO: | ELVKKGCWLDDFNCYDRQECVETEENPQVYFCCCEGNFCNEKFSYFPQMEV |
| | 97) with 5 additional aa at N- | TQPTSNPVTPKPP |
| | terminus | |
| 1491 | N24Q mutant of Hybrid hu- | SGRGEAETRECIYYNANWELERTQQSGLERCEGDKDKRLHCYASWRNSSGT |
| | ActRIIB-ECD (SEQ ID NO: | IELVKKGCWLDDFNCYDRQECVETEENPQVYFCCCEGNFCNEKFSYFPQME |
| | 97) with 6 additional aa at N- | VTQPTSNPVTPKPP |
| | terminus | |
| 1492 | N19Q mutant of Hybrid hu- | AETRECIYYNANWELERTQQSGLERCEGEKDKRLHCYASWRNSSGTIELVK |
| | ActRIIB-ECD (SEQ ID NO: | KGCWLDDFNCYDRQECVAKEENPQVYFCCCEGNFCNEKFSYFPQMEVTQP |
| | 98) with 1 additional aa at N- | TSNPVTPKPP |
| | terminus | |
| 1493 | N20Q mutant of Hybrid hu- | EAETRECIYYNANWELERTQQSGLERCEGEKDKRLHCYASWRNSSGTIELV |
| | ActRIIB-ECD (SEQ ID NO: | KKGCWLDDFNCYDRQECVAKEENPQVYFCCCEGNFCNEKFSYFPQMEVTQ |
| | 98) with 2 additional aa at N- | PTSNPVTPKPP |
| | terminus | |
| 1494 | N21Q mutant of Hybrid hu- | GEAETRECIYYNANWELERTQQSGLERCEGEKDKRLHCYASWRNSSGTIEL |
| | ActRIIB-ECD (SEQ ID NO: | VKKGCWLDDFNCYDRQECVAKEENPQVYFCCCEGNFCNEKFSYFPQMEVT |
| | 98) with 3 additional aa at N- | QPTSNPVTPKPP |
| | terminus | |
| 1495 | N22Q mutant of Hybrid hu- | RGEAETRECIYYNANWELERTQQSGLERCEGEKDKRLHCYASWRNSSGTIE |
| | ActRIIB-ECD (SEQ ID NO: | LVKKGCWLDDFNCYDRQECVAKEENPQVYFCCCEGNFCNEKFSYFPQMEV |
| | 98) with 4 additional aa at N- | TQPTSNPVTPKPP |
| | terminus | |
| 1496 | N23Q mutant of Hybrid hu- | GRGEAETRECIYYNANWELERTQQSGLERCEGEKDKRLHCYASWRNSSGTI |
| | ActRIIB-ECD (SEQ ID NO: | ELVKKGCWLDDFNCYDRQECVAKEENPQVYFCCCEGNFCNEKFSYFPQME |
| | 98) with 5 additional aa at N- | VTQPTSNPVTPKPP |
| | terminus | |
| 1497 | N24Q mutant of Hybrid hu- | SGRGEAETRECIYYNANWELERTQQSGLERCEGEKDKRLHCYASWRNSSGT |
| | ActRIIB-ECD (SEQ ID NO: | IELVKKGCWLDDFNCYDRQECVAKEENPQVYFCCCEGNFCNEKFSYFPQME |
| | 98) with 6 additional aa at N- | VTQPTSNPVTPKPP |
| | terminus | |
| 1498 | N19Q mutant of Hybrid hu- | AETRECIYYNANWELERTQQSGLERCEGDQDKRLHCYASWRNSSGTIELVK |
| | ActRIIB-ECD (SEQ ID NO: | KGCWLDDFNCYDRQECVATKENPQVYFCCCEGNFCNEKFSYFPQMEVTQP |
| | 99) with 1 additional aa at N- | TSNPVTPKPP |
| | terminus | |
| 1499 | N20Q mutant of Hybrid hu- | EAETRECIYYNANWELERTQQSGLERCEGDQDKRLHCYASWRNSSGTIELV |
| | ActRIIB-ECD (SEQ ID NO: | KKGCWLDDFNCYDRQECVATKENPQVYFCCCEGNFCNEKFSYFPQMEVTQ |
| | 99) with 2 additional aa at N- | PTSNPVTPKPP |
| | terminus | |
| 1500 | N21Q mutant of Hybrid hu- | GEAETRECIYYNANWELERTQQSGLERCEGDQDKRLHCYASWRNSSGTIEL |
| | ActRIIB-ECD (SEQ ID NO: | VKKGCWLDDFNCYDRQECVATKENPQVYFCCCEGNFCNEKFSYFPQMEVT |
| | 99) with 3 additional aa at N- | QPTSNPVTPKPP |
| | terminus | |
| 1501 | N22Q mutant of Hybrid hu- | RGEAETRECIYYNANWELERTQQSGLERCEGDQDKRLHCYASWRNSSGTIE |
| | ActRIIB-ECD (SEQ ID NO: | LVKKGCWLDDFNCYDRQECVATKENPQVYFCCCEGNFCNEKFSYFPQMEV |
| | 99) with 4 additional aa at N- | TQPTSNPVTPKPP |
| | terminus | |
| 1502 | N23Q mutant of Hybrid hu- | GRGEAETRECIYYNANWELERTQQSGLERCEGDQDKRLHCYASWRNSSGTI |
| | ActRIIB-ECD (SEQ ID NO: | ELVKKGCWLDDFNCYDRQECVATKENPQVYFCCCEGNFCNEKFSYFPQME |
| | 99) with 5 additional aa at N- | VTQPTSNPVTPKPP |
| | terminus | |
| 1503 | N24Q mutant of Hybrid hu- | SGRGEAETRECIYYNANWELERTQQSGLERCEGDQDKRLHCYASWRNSSGT |
| | ActRIIB-ECD (SEQ ID NO: | IELVKKGCWLDDFNCYDRQECVATKENPQVYFCCCEGNFCNEKFSYFPQME |
| | 99) with 6 additional aa at N- | VTQPTSNPVTPKPP |
| | terminus | |
| 1504 | N19Q mutant of Hybrid hu- | AETRECIYYNANWELERTQQSGLERCEGDKDKRLHCYASWRNSSGTIELVK |
| | ActRIIB-ECD (SEQ ID NO: | QGCWLDDFNCYDRQECVAKKENPQVYFCCCEGNFCNEKFSYFPQMEVTQP |
| | 100) with 1 additional aa at | TSNPVTPKPP |
| | N-terminus | |
| 1505 | N20Q mutant of Hybrid hu- | EAETRECIYYNANWELERTQQSGLERCEGDKDKRLHCYASWRNSSGTIELV |
| | ActRIIB-ECD (SEQ ID NO: | KQGCWLDDFNCYDRQECVAKKENPQVYFCCCEGNFCNEKFSYFPQMEVTQ |
| | 100) with 2 additional aa at | PTSNPVTPKPP |
| | N-terminus | |
| 1506 | N21Q mutant of Hybrid hu- | GEAETRECIYYNANWELERTQQSGLERCEGDKDKRLHCYASWRNSSGTIEL |
| | ActRIIB-ECD (SEQ ID NO: | VKQGCWLDDFNCYDRQECVAKKENPQVYFCCCEGNFCNEKFSYFPQMEVT |
| | 100) with 3 additional aa at | QPTSNPVTPKPP |
| | N-terminus | |
| 1507 | N22Q mutant of Hybrid hu- | RGEAETRECIYYNANWELERTQQSGLERCEGDKDKRLHCYASWRNSSGTIE |
| | ActRIIB-ECD (SEQ ID NO: | LVKQGCWLDDFNCYDRQECVAKKENPQVYFCCCEGNFCNEKFSYFPQMEV |
| | 100) with 4 additional aa at | TQPTSNPVTPKPP |
| | N-terminus | |
| 1508 | N23Q mutant of Hybrid hu- | GRGEAETRECIYYNANWELERTQQSGLERCEGDKDKRLHCYASWRNSSGTI |
| | ActRIIB-ECD (SEQ ID NO: | ELVKQGCWLDDFNCYDRQECVAKKENPQVYFCCCEGNFCNEKFSYFPQME |
| | 100) with 5 additional aa at | VTQPTSNPVTPKPP |
| | N-terminus | |
| 1509 | N24Q mutant of Hybrid hu- | SGRGEAETRECIYYNANWELERTQQSGLERCEGDKDKRLHCYASWRNSSGT |
| | ActRIIB-ECD (SEQ ID NO: | IELVKQGCWLDDFNCYDRQECVAKKENPQVYFCCCEGNFCNEKFSYFPQME |
| | 100) with 6 additional aa at | VTQPTSNPVTPKPP |
| | N-terminus | |
| 1510 | N19Q mutant of Hybrid hu- | AETRECIYYNANWELERTQQSGLERCEGDKDKRLHCYASWRNSSGTIEIVK |
| | ActRIIB-ECD (SEQ ID NO: | QGCWLDDFNCYDRQECVAEKENPQVYFCCCEGNFCNEKFSYFPQMEVTQP |
| | 101) with 1 additional aa at | TSNPVTPKPP |
| | N-terminus | |
| 1511 | N20Q mutant of Hybrid hu- | EAETRECIYYNANWELERTQQSGLERCEGDKDKRLHCYASWRNSSGTIEIVK |
| | ActRIIB-ECD (SEQ ID NO: | QGCWLDDFNCYDRQECVAEKENPQVYFCCCEGNFCNEKFSYFPQMEVTQP |
| | 101) with 2 additional aa at | TSNPVTPKPP |
| | N-terminu | |
| 1512 | N21Q mutant of Hybrid hu- | GEAETRECIYYNANWELERTQQSGLERCEGDKDKRLHCYASWRNSSGTIEIV |
| | ActRIIB-ECD (SEQ ID NO: | KQGCWLDDFNCYDRQECVAEKENPQVYFCCCEGNFCNEKFSYFPQMEVTQ |
| | 101) with 3 additional aa at | PTSNPVTPKPP |
| | N-terminus | |
| 1513 | N22Q mutant of Hybrid hu- | RGEAETRECIYYNANWELERTQQSGLERCEGDKDKRLHCYASWRNSSGTIEI |
| | ActRIIB-ECD (SEQ ID NO: | VKQGCWLDDFNCYDRQECVAEKENPQVYFCCCEGNFCNEKFSYFPQMEVT |
| | 101) with 4 additional aa at | QPTSNPVTPKPP |
| | N-terminus | |
| 1514 | N23Q mutant of Hybrid hu- | GRGEAETRECIYYNANWELERTQQSGLERCEGDKDKRLHCYASWRNSSGTI |
| | ActRIIB-ECD (SEQ ID NO: | EIVKQGCWLDDFNCYDRQECVAEKENPQVYFCCCEGNFCNEKFSYFPQMEV |
| | 101) with 5 additional aa at | TQPTSNPVTPKPP |
| | N-terminus | |
| 1515 | N24Q mutant of Hybrid hu- | SGRGEAETRECIYYNANWELERTQQSGLERCEGDKDKRLHCYASWRNSSGT |
| | ActRIIB-ECD (SEQ ID NO: | IEIVKQGCWLDDFNCYDRQECVAEKENPQVYFCCCEGNFCNEKFSYFPQME |
| | 101) with 6 additional aa at | VTQPTSNPVTPKPP |
| | N-terminus | |
| 1516 | N19Q mutant of Hybrid hu- | AETRECIYYNANWELERTQQSGLERCYGDQDKRLHCYASWRNSSGSIEIVK |
| | ActRIIB-ECD (SEQ ID NO: | QGCWLDDFNCYDRQECVAKKENPQVYFCCCEGNFCNEKFSYFPQMEVTQP |
| | 102) with 1 additional aa at | TSNPVTPKPP |
| | N-terminus | |
| 1517 | N20Q mutant of Hybrid hu- | EAETRECIYYNANWELERTQQSGLERCYGDQDKRLHCYASWRNSSGSIEIVK |
| | ActRIIB-ECD (SEQ ID NO: | QGCWLDDFNCYDRQECVAKKENPQVYFCCCEGNFCNEKFSYFPQMEVTQP |
| | 102) with 2 additional aa at | TSNPVTPKPP |
| | N-terminus | |
| 1518 | N21Q mutant of Hybrid hu- | GEAETRECIYYNANWELERTQQSGLERCYGDQDKRLHCYASWRNSSGSIEIV |
| | ActRIIB-ECD (SEQ ID NO: | KQGCWLDDFNCYDRQECVAKKENPQVYFCCCEGNFCNEKFSYFPQMEVTQ |
| | 102) with 3 additional aa at | PTSNPVTPKPP |
| | N-terminus | |
| 1519 | N22Q mutant of Hybrid hu- | RGEAETRECIYYNANWELERTQQSGLERCYGDQDKRLHCYASWRNSSGSIEI |
| | ActRIIB-ECD (SEQ ID NO: | VKQGCWLDDFNCYDRQECVAKKENPQVYFCCCEGNFCNEKFSYFPQMEVT |
| | 102) with 4 additional aa at | QPTSNPVTPKPP |
| | N-terminus | |
| 1520 | N23Q mutant of Hybrid hu- | GRGEAETRECIYYNANWELERTQQSGLERCYGDQDKRLHCYASWRNSSGSI |
| | ActRIIB-ECD (SEQ ID NO: | EIVKQGCWLDDFNCYDRQECVAKKENPQVYFCCCEGNFCNEKFSYFPQME |
| | 102) with 5 additional aa at | VTQPTSNPVTPKPP |
| | N-terminus | |
| 1521 | N24Q mutant of Hybrid hu- | SGRGEAETRECIYYNANWELERTQQSGLERCYGDQDKRLHCYASWRNSSGS |
| | ActRIIB-ECD (SEQ ID NO: | IEIVKQGCWLDDFNCYDRQECVAKKENPQVYFCCCEGNFCNEKFSYFPQME |
| | 102) with 6 additional aa at | VTQPTSNPVTPKPP |
| | N-terminus | |
| 1522 | N19Q mutant of Hybrid hu- | AETRECIYYNANWELERTQQSGLERCEGEKDKRRHCYASWRNSSGTIEIVKK |
| | ActRIIB-ECD (SEQ ID NO: | GCWLDDFNCYDRQECVATKENPQVYFCCCEGNFCNEKFSYFPQMEVTQPTS |
| | 103) with 1 additional aa at | NPVTPKPP |
| | N-terminus | |
| 1523 | N20Q mutant of Hybrid hu- | EAETRECIYYNANWELERTQQSGLERCEGEKDKRRHCYASWRNSSGTIEIVK |
| | ActRIIB-ECD (SEQ ID NO: | KGCWLDDFNCYDRQECVATKENPQVYFCCCEGNFCNEKFSYFPQMEVTQP |
| | 103) with 2 additional aa at | TSNPVTPKPP |
| | N-terminus | |
| 1524 | N21Q mutant of Hybrid hu- | GEAETRECIYYNANWELERTQQSGLERCEGEKDKRRHCYASWRNSSGTIEIV |
| | ActRIIB-ECD (SEQ ID NO: | KKGCWLDDFNCYDRQECVATKENPQVYFCCCEGNFCNEKFSYFPQMEVTQ |
| | 103) with 3 additional aa at | PTSNPVTPKPP |
| | N-terminus | |
| 1525 | N22Q mutant of Hybrid hu- | RGEAETRECIYYNANWELERTQQSGLERCEGEKDKRRHCYASWRNSSGTIEI |
| | ActRIIB-ECD (SEQ ID NO: | VKKGCWLDDFNCYDRQECVATKENPQVYFCCCEGNFCNEKFSYFPQMEVT |
| | 103) with 4 additional aa at | QPTSNPVTPKPP |
| | N-terminus | |
| 1526 | N23Q mutant of Hybrid hu- | GRGEAETRECIYYNANWELERTQQSGLERCEGEKDKRRHCYASWRNSSGTI |
| | ActRIIB-ECD (SEQ ID NO: | EIVKKGCWLDDFNCYDRQECVATKENPQVYFCCCEGNFCNEKFSYFPQMEV |
| | 103) with 5 additional aa at | TQPTSNPVTPKPP |
| | N-terminus | |
| 1527 | N24Q mutant of Hybrid hu- | SGRGEAETRECIYYNANWELERTQQSGLERCEGEKDKRRHCYASWRNSSGT |
| | ActRIIB-ECD (SEQ ID NO: | IEIVKKGCWLDDFNCYDRQECVATKENPQVYFCCCEGNFCNEKFSYFPQME |
| | 103) with 5 additional aa at | VTQPTSNPVTPKPP |
| | N-terminus | |
| 1528 | N19Q mutant of Hybrid hu- | AETRECIYYNANWELERTQQSGLERCYGDQDKRRHCYASWRNSSGTIELVK |
| | ActRIIB-ECD (SEQ ID NO: | KGCWLDDFNCYDRQECVATEENPEVYFCCCEGNFCNEKFSYFPQMEVTQPT |
| | 104) with 1 additional aa at | SNPVTPKPP |
| | N-terminus | |
| 1529 | N20Q mutant of Hybrid hu- | EAETRECIYYNANWELERTQQSGLERCYGDQDKRRHCYASWRNSSGTIELV |
| | ActRIIB-ECD (SEQ ID NO: | KKGCWLDDFNCYDRQECVATEENPEVYFCCCEGNFCNEKFSYFPQMEVTQP |
| | 104) with 2 additional aa at | TSNPVTPKPP |
| | N-terminus | |
| 1530 | N21Q mutant of Hybrid hu- | GEAETRECIYYNANWELERTQQSGLERCYGDQDKRRHCYASWRNSSGTIEL |
| | ActRIIB-ECD (SEQ ID NO: | VKKGCWLDDFNCYDRQECVATEENPEVYFCCCEGNFCNEKFSYFPQMEVT |
| | 104) with 3 additional aa at | QPTSNPVTPKPP |
| | N-terminus | |
| 1531 | N22Q mutant of Hybrid hu- | RGEAETRECIYYNANWELERTQQSGLERCYGDQDKRRHCYASWRNSSGTIE |
| | ActRIIB-ECD (SEQ ID NO: | LVKKGCWLDDFNCYDRQECVATEENPEVYFCCCEGNFCNEKFSYFPQMEVT |
| | 104) with 4 additional aa at | QPTSNPVTPKPP |
| | N-terminus | |
| 1532 | N23Q mutant of Hybrid hu- | GRGEAETRECIYYNANWELERTQQSGLERCYGDQDKRRHCYASWRNSSGTI |
| | ActRIIB-ECD (SEQ ID NO: | ELVKKGCWLDDFNCYDRQECVATEENPEVYFCCCEGNFCNEKFSYFPQMEV |
| | 104) with 5 additional aa at | TQPTSNPVTPKPP |
| | N-terminus | |
| 1533 | N24Q mutant of Hybrid hu- | SGRGEAETRECIYYNANWELERTQQSGLERCYGDQDKRRHCYASWRNSSG |
| | ActRIIB-ECD (SEQ ID NO: | TIELVKKGCWLDDFNCYDRQECVATEENPEVYFCCCEGNFCNEKFSYFPQM |
| | 104) with 6 additional aa at | EVTQPTSNPVTPKPP |
| | N-terminus | |
| 1534 | N19Q mutant of Hybrid hu- | AETRECIYYNANWELERTQQSGLERCEGEQDKRRHCYASWRNSSGTIELVK |
| | ActRIIB-ECD (SEQ ID NO: | KGCWLDDFNCYDRQECVATEENPQVYFCCCEGNFCNEKFSYFPQMEVTQPT |
| | 105) with 1 additional aa at | SNPVTPKPP |
| | N-terminus | |
| 1535 | N20Q mutant of Hybrid hu- | EAETRECIYYNANWELERTQQSGLERCEGEQDKRRHCYASWRNSSGTIELV |
| | ActRIIB-ECD (SEQ ID NO: | KKGCWLDDFNCYDRQECVATEENPQVYFCCCEGNFCNEKFSYFPQMEVTQ |
| | 105) with 2 additional aa at | PTSNPVTPKPP |
| | N-terminus | |
| 1536 | N21Q mutant of Hybrid hu- | GEAETRECIYYNANWELERTQQSGLERCEGEQDKRRHCYASWRNSSGTIEL |
| | ActRIIB-ECD (SEQ ID NO: | VKKGCWLDDFNCYDRQECVATEENPQVYFCCCEGNFCNEKFSYFPQMEVT |
| | 105) with 3 additional aa at | QPTSNPVTPKPP |
| | N-terminus | |
| 1537 | N22Q mutant of Hybrid hu- | RGEAETRECIYYNANWELERTQQSGLERCEGEQDKRRHCYASWRNSSGTIE |
| | ActRIIB-ECD (SEQ ID NO: | LVKKGCWLDDFNCYDRQECVATEENPQVYFCCCEGNFCNEKFSYFPQMEV |
| | 105) with 4 additional aa at | TQPTSNPVTPKPP |
| | N-terminus | |
| 1538 | N23Q mutant of Hybrid hu- | GRGEAETRECIYYNANWELERTQQSGLERCEGEQDKRRHCYASWRNSSGTI |
| | ActRIIB-ECD (SEQ ID NO: | ELVKKGCWLDDFNCYDRQECVATEENPQVYFCCCEGNFCNEKFSYFPQME |
| | 105) with 5 additional aa at | VTQPTSNPVTPKPP |
| | N-terminus | |
| 1539 | N24Q mutant of Hybrid hu- | SGRGEAETRECIYYNANWELERTQQSGLERCEGEQDKRRHCYASWRNSSGT |
| | ActRIIB-ECD (SEQ ID NO: | IELVKKGCWLDDFNCYDRQECVATEENPQVYFCCCEGNFCNEKFSYFPQME |
| | 105) with 6 additional aa at | VTQPTSNPVTPKPP |
| | N-terminus | |
| 1540 | N19Q mutant of Hybrid hu- | AETRECIYYNANWELERTQQSGLERCYGEQDKRLHCYASWRNSSGSIEIVKK |
| | ActRIIB-ECD (SEQ ID NO: | GCWLDDFNCYDRTDCVATEENPQVYFCCCEGNFCNEKFSYFPQMEVTQPTS |
| | 106) with 1 additional aa at | NPVTPKPP |
| | N-terminus | |
| 1541 | N20Q mutant of Hybrid hu- | EAETRECIYYNANWELERTQQSGLERCYGEQDKRLHCYASWRNSSGSIEIVK |
| | ActRIIB-ECD (SEQ ID NO: | KGCWLDDFNCYDRTDCVATEENPQVYFCCCEGNFCNEKFSYFPQMEVTQPT |
| | 106) with 2 additional aa at | SNPVTPKPP |
| | N-terminus | |
| 1542 | N21Q mutant of Hybrid hu- | GEAETRECIYYNANWELERTQQSGLERCYGEQDKRLHCYASWRNSSGSIEIV |
| | ActRIIB-ECD (SEQ ID NO: | KKGCWLDDFNCYDRTDCVATEENPQVYFCCCEGNFCNEKFSYFPQMEVTQ |
| | 106) with 3 additional aa at | PTSNPVTPKPP |
| | N-terminus | |
| 1543 | N22Q mutant of Hybrid hu- | RGEAETRECIYYNANWELERTQQSGLERCYGEQDKRLHCYASWRNSSGSIEI |
| | ActRIIB-ECD (SEQ ID NO: | VKKGCWLDDFNCYDRTDCVATEENPQVYFCCCEGNFCNEKFSYFPQMEVT |
| | 106) with 4 additional aa at | QPTSNPVTPKPP |
| | N-terminus | |
| 1544 | N23Q mutant of Hybrid hu- | GRGEAETRECIYYNANWELERTQQSGLERCYGEQDKRLHCYASWRNSSGSI |
| | ActRIIB-ECD (SEQ ID NO: | EIVKKGCWLDDFNCYDRTDCVATEENPQVYFCCCEGNFCNEKFSYFPQMEV |
| | 106) with 5 additional aa at | TQPTSNPVTPKPP |
| | N-terminus | |
| 1545 | N24Q mutant of Hybrid hu- | SGRGEAETRECIYYNANWELERTQQSGLERCYGEQDKRLHCYASWRNSSGS |
| | ActRIIB-ECD (SEQ ID NO: | IEIVKKGCWLDDFNCYDRTDCVATEENPQVYFCCCEGNFCNEKFSYFPQME |
| | 106) with 6 additional aa at | VTQPTSNPVTPKPP |
| | N-terminus | |
| 1546 | N19Q mutant of Hybrid hu- | AETRECIYYNANWELERTQQSGLERCEGEQDKRRHCYASWRNSSGSIELVK |
| | ActRIIB-ECD (SEQ ID NO: | KGCWLDDFNCYDRQECVAKEENPQVYFCCCEGNFCNEKFSYFPQMEVTQP |
| | 107) with 1 additional aa at | TSNPVTPKPP |
| | N-terminus | |
| 1547 | N20Q mutant of Hybrid hu- | EAETRECIYYNANWELERTQQSGLERCEGEQDKRRHCYASWRNSSGSIELV |
| | ActRIIB-ECD (SEQ ID NO: | KKGCWLDDFNCYDRQECVAKEENPQVYFCCCEGNFCNEKFSYFPQMEVTQ |
| | 107) with 2 additional aa at | PTSNPVTPKPP |
| | N-terminus | |
| 1548 | N21Q mutant of Hybrid hu- | GEAETRECIYYNANWELERTQQSGLERCEGEQDKRRHCYASWRNSSGSIEL |
| | ActRIIB-ECD (SEQ ID NO: | VKKGCWLDDFNCYDRQECVAKEENPQVYFCCCEGNFCNEKFSYFPQMEVT |
| | 107) with 3 additional aa at | QPTSNPVTPKPP |
| | N-terminus | |
| 1549 | N22Q mutant of Hybrid hu- | RGEAETRECIYYNANWELERTQQSGLERCEGEQDKRRHCYASWRNSSGSIE |
| | ActRIIB-ECD (SEQ ID NO: | LVKKGCWLDDFNCYDRQECVAKEENPQVYFCCCEGNFCNEKFSYFPQMEV |
| | 107) with 4 additional aa at | TQPTSNPVTPKPP |
| | N-terminus | |
| 1550 | N23Q mutant of Hybrid hu- | GRGEAETRECIYYNANWELERTQQSGLERCEGEQDKRRHCYASWRNSSGSI |
| | ActRIIB-ECD (SEQ ID NO: | ELVKKGCWLDDFNCYDRQECVAKEENPQVYFCCCEGNFCNEKFSYFPQME |
| | 107) with 5 additional aa at | VTQPTSNPVTPKPP |
| | N-terminus | |
| 1551 | N24Q mutant of Hybrid hu- | SGRGEAETRECIYYNANWELERTQQSGLERCEGEQDKRRHCYASWRNSSGS |
| | ActRIIB-ECD (SEQ ID NO: | IELVKKGCWLDDFNCYDRQECVAKEENPQVYFCCCEGNFCNEKFSYFPQME |
| | 107) with 6 additional aa at | VTQPTSNPVTPKPP |
| | N-terminus | |
| 1552 | N19Q mutant of Hybrid hu- | AETRECIYYNANWELERTQQSGLERCEGEQDKRRHCYASWRNSSGTIEIVKK |
| | ActRIIB-ECD (SEQ ID NO: | GCWLDDFNCYDRQECVAKEENPQVYFCCCEGNFCNEKFSYFPQMEVTQPTS |
| | 108) with 1 additional aa at | NPVTPKPP |
| | N-terminus | |
| 1553 | N20Q mutant of Hybrid hu- | EAETRECIYYNANWELERTQQSGLERCEGEQDKRRHCYASWRNSSGTIEIVK |
| | ActRIIB-ECD (SEQ ID NO: | KGCWLDDFNCYDRQECVAKEENPQVYFCCCEGNFCNEKFSYFPQMEVTQP |
| | 108) with 2 additional aa at | TSNPVTPKPP |
| | N-terminus | |
| 1554 | N21Q mutant of Hybrid hu- | GEAETRECIYYNANWELERTQQSGLERCEGEQDKRRHCYASWRNSSGTIEIV |
| | ActRIIB-ECD (SEQ ID NO: | KKGCWLDDFNCYDRQECVAKEENPQVYFCCCEGNFCNEKFSYFPQMEVTQ |
| | 108) with 3 additional aa at | PTSNPVTPKPP |
| | N-terminus | |
| 1555 | N22Q mutant of Hybrid hu- | RGEAETRECIYYNANWELERTQQSGLERCEGEQDKRRHCYASWRNSSGTIEI |
| | ActRIIB-ECD (SEQ ID NO: | VKKGCWLDDFNCYDRQECVAKEENPQVYFCCCEGNFCNEKFSYFPQMEVT |
| | 108) with 4 additional aa at | QPTSNPVTPKPP |
| | N-terminus | |
| 1556 | N23Q mutant of Hybrid hu- | GRGEAETRECIYYNANWELERTQQSGLERCEGEQDKRRHCYASWRNSSGTI |
| | ActRIIB-ECD (SEQ ID NO: | EIVKKGCWLDDFNCYDRQECVAKEENPQVYFCCCEGNFCNEKFSYFPQMEV |
| | 108) with 5 additional aa at | TQPTSNPVTPKPP |
| | N-terminus | |
| 1557 | N24Q mutant of Hybrid hu- | SGRGEAETRECIYYNANWELERTQQSGLERCEGEQDKRRHCYASWRNSSGT |
| | ActRIIB-ECD (SEQ ID NO: | IEIVKKGCWLDDFNCYDRQECVAKEENPQVYFCCCEGNFCNEKFSYFPQME |
| | 108) with 6 additional aa at | VTQPTSNPVTPKPP |
| | N-terminus | |
| 1558 | N19Q mutant of Hybrid hu- | AETRECIYYNANWELERTQQSGLERCEGEQDKRRHCYASWRNSSGSIEIVKK |
| | ActRIIB-ECD (SEQ ID NO: | GCWLDDFNCYDRQECVATKENPQVYFCCCEGNFCNEKFSYFPQMEVTQPTS |
| | 109) with 1 additional aa at | NPVTPKPP |
| | N-terminus | |
| 1559 | N20Q mutant of Hybrid hu- | EAETRECIYYNANWELERTQQSGLERCEGEQDKRRHCYASWRNSSGSIEIVK |
| | ActRIIB-ECD (SEQ ID NO: | KGCWLDDFNCYDRQECVATKENPQVYFCCCEGNFCNEKFSYFPQMEVTQP |
| | 109) with 2 additional aa at | TSNPVTPKPP |
| | N-terminus | |
| 1560 | N21Q mutant of Hybrid hu- | GEAETRECIYYNANWELERTQQSGLERCEGEQDKRRHCYASWRNSSGSIEIV |
| | ActRIIB-ECD (SEQ ID NO: | KKGCWLDDFNCYDRQECVATKENPQVYFCCCEGNFCNEKFSYFPQMEVTQ |
| | 109) with 3 additional aa at | PTSNPVTPKPP |
| | N-terminus | |
| 1561 | N22Q mutant of Hybrid hu- | RGEAETRECIYYNANWELERTQQSGLERCEGEQDKRRHCYASWRNSSGSIEI |
| | ActRIIB-ECD (SEQ ID NO: | VKKGCWLDDFNCYDRQECVATKENPQVYFCCCEGNFCNEKFSYFPQMEVT |
| | 109) with 4 additional aa at | QPTSNPVTPKPP |
| | N-terminus | |
| 1562 | N23Q mutant of Hybrid hu- | GRGEAETRECIYYNANWELERTQQSGLERCEGEQDKRRHCYASWRNSSGSI |
| | ActRIIB-ECD (SEQ ID NO: | EIVKKGCWLDDFNCYDRQECVATKENPQVYFCCCEGNFCNEKFSYFPQMEV |
| | 109) with 5 additional aa at | TQPTSNPVTPKPP |
| | N-terminus | |
| 1563 | N24Q mutant of Hybrid hu- | SGRGEAETRECIYYNANWELERTQQSGLERCEGEQDKRRHCYASWRNSSGS |
| | ActRIIB-ECD (SEQ ID NO: | IEIVKKGCWLDDFNCYDRQECVATKENPQVYFCCCEGNFCNEKFSYFPQME |
| | 109) with 6 additional aa at | VTQPTSNPVTPKPP |
| | N-terminus | |
| 1564 | N19Q mutant of Hybrid hu- | AETRECIYYNANWELERTQQSGLERCEGDQDKRLHCYASWRNSSGTIEIVK |
| | ActRIIB-ECD (SEQ ID NO: | KGCWLDDFNCYDRQECVATKENPQVYFCCCEGNFCNEKFSYFPQMEVTQP |
| | 110) with 1 additional aa at | TSNPVTPKPP |
| | N-terminus | |
| 1565 | N20Q mutant of Hybrid hu- | EAETRECIYYNANWELERTQQSGLERCEGDQDKRLHCYASWRNSSGTIEIVK |
| | ActRIIB-ECD (SEQ ID NO: | KGCWLDDFNCYDRQECVATKENPQVYFCCCEGNFCNEKFSYFPQMEVTQP |
| | 110) with 2 additional aa at | TSNPVTPKPP |
| | N-terminus | |
| 1566 | N21Q mutant of Hybrid hu- | GEAETRECIYYNANWELERTQQSGLERCEGDQDKRLHCYASWRNSSGTIEIV |
| | ActRIIB-ECD (SEQ ID NO: | KKGCWLDDFNCYDRQECVATKENPQVYFCCCEGNFCNEKFSYFPQMEVTQ |
| | 110) with 3 additional aa at | PTSNPVTPKPP |
| | N-terminus | |
| 1567 | N22Q mutant of Hybrid hu- | RGEAETRECIYYNANWELERTQQSGLERCEGDQDKRLHCYASWRNSSGTIEI |
| | ActRIIB-ECD (SEQ ID NO: | VKKGCWLDDFNCYDRQECVATKENPQVYFCCCEGNFCNEKFSYFPQMEVT |
| | 110) with 4 additional aa at | QPTSNPVTPKPP |
| | N-terminus | |
| 1568 | N23Q mutant of Hybrid hu- | GRGEAETRECIYYNANWELERTQQSGLERCEGDQDKRLHCYASWRNSSGTI |
| | ActRIIB-ECD (SEQ ID NO: | EIVKKGCWLDDFNCYDRQECVATKENPQVYFCCCEGNFCNEKFSYFPQMEV |
| | 110) with 5 additional aa at | TQPTSNPVTPKPP |
| | N-terminus | |
| 1569 | N24Q mutant of Hybrid hu- | SGRGEAETRECIYYNANWELERTQQSGLERCEGDQDKRLHCYASWRNSSGT |
| | ActRIIB-ECD (SEQ ID NO: | IEIVKKGCWLDDFNCYDRQECVATKENPQVYFCCCEGNFCNEKFSYFPQME |
| | 110) with 6 additional aa at | VTQPTSNPVTPKPP |
| | N-terminus | |
| 1570 | N19Q mutant of Hybrid hu- | AETRECIYYNANWELERTQQSGLERCEGDQDKRLHCYASWRNSSGSIELVK |
| | ActRIIB-ECD (SEQ ID NO: | KGCWLDDFNCYDRQECVATKENPQVYFCCCEGNFCNEKFSYFPQMEVTQP |
| | 111) with 1 additional aa at | TSNPVTPKPP |
| | N-terminus | |
| 1571 | N20Q mutant of Hybrid hu- | EAETRECIYYNANWELERTQQSGLERCEGDQDKRLHCYASWRNSSGSIELV |
| | ActRIIB-ECD (SEQ ID NO: | KKGCWLDDFNCYDRQECVATKENPQVYFCCCEGNFCNEKFSYFPQMEVTQ |
| | 111) with 2 additional aa at | PTSNPVTPKPP |
| | N-terminus | |
| 1572 | N21Q mutant of Hybrid hu- | GEAETRECIYYNANWELERTQQSGLERCEGDQDKRLHCYASWRNSSGSIEL |
| | ActRIIB-ECD (SEQ ID NO: | VKKGCWLDDFNCYDRQECVATKENPQVYFCCCEGNFCNEKFSYFPQMEVT |
| | 111) with 3 additional aa at | QPTSNPVTPKPP |
| | N-terminus | |
| 1573 | N22Q mutant of Hybrid hu- | RGEAETRECIYYNANWELERTQQSGLERCEGDQDKRLHCYASWRNSSGSIE |
| | ActRIIB-ECD (SEQ ID NO: | LVKKGCWLDDFNCYDRQECVATKENPQVYFCCCEGNFCNEKFSYFPQMEV |
| | 111) with 4 additional aa at | TQPTSNPVTPKPP |
| | N-terminus | |
| 1574 | N23Q mutant of Hybrid hu- | GRGEAETRECIYYNANWELERTQQSGLERCEGDQDKRLHCYASWRNSSGSI |
| | ActRIIB-ECD (SEQ ID NO: | ELVKKGCWLDDFNCYDRQECVATKENPQVYFCCCEGNFCNEKFSYFPQME |
| | 111) with 5 additional aa at | VTQPTSNPVTPKPP |
| | N-terminus | |
| 1575 | N24Q mutant of Hybrid hu- | SGRGEAETRECIYYNANWELERTQQSGLERCEGDQDKRLHCYASWRNSSGS |
| | ActRIIB-ECD (SEQ ID NO: | IELVKKGCWLDDFNCYDRQECVATKENPQVYFCCCEGNFCNEKFSYFPQME |
| | 111) with 6 additional aa at | VTQPTSNPVTPKPP |
| | N-terminus | |
| 1576 | N19Q mutant of Hybrid hu- | AETRECIYYNANWELERTQQSGLERCEGDQDKRLHCYASWRNSSGTIELVK |
| | ActRIIB-ECD (SEQ ID NO: | KGCWLDDFNCYDRQECVATKENPQVYFCCCEGNFCNEKFSYFPQMEVTQP |
| | 112) with 1 additional aa at | TSNPVTPKPP |
| | N-terminus | |
| 1577 | N20Q mutant of Hybrid hu- | EAETRECIYYNANWELERTQQSGLERCEGDQDKRLHCYASWRNSSGTIELV |
| | ActRIIB-ECD (SEQ ID NO: | KKGCWLDDFNCYDRQECVATKENPQVYFCCCEGNFCNEKFSYFPQMEVTQ |
| | 112) with 2 additional aa at | PTSNPVTPKPP |
| | N-terminus | |
| 1578 | N21Q mutant of Hybrid hu- | GEAETRECIYYNANWELERTQQSGLERCEGDQDKRLHCYASWRNSSGTIEL |
| | ActRIIB-ECD (SEQ ID NO: | VKKGCWLDDFNCYDRQECVATKENPQVYFCCCEGNFCNEKFSYFPQMEVT |
| | 112) with 3 additional aa at | QPTSNPVTPKPP |
| | N-terminus | |
| 1579 | N22Q mutant of Hybrid hu- | RGEAETRECIYYNANWELERTQQSGLERCEGDQDKRLHCYASWRNSSGTIE |
| | ActRIIB-ECD (SEQ ID NO: | LVKKGCWLDDFNCYDRQECVATKENPQVYFCCCEGNFCNEKFSYFPQMEV |
| | 112) with 4 additional aa at | TQPTSNPVTPKPP |
| | N-terminus | |
| 1580 | N23Q mutant of Hybrid hu- | GRGEAETRECIYYNANWELERTQQSGLERCEGDQDKRLHCYASWRNSSGTI |
| | ActRIIB-ECD (SEQ ID NO: | ELVKKGCWLDDFNCYDRQECVATKENPQVYFCCCEGNFCNEKFSYFPQME |
| | 112) with 5 additional aa at | VTQPTSNPVTPKPP |
| | N-terminus | |
| 1581 | N24Q mutant of Hybrid hu- | SGRGEAETRECIYYNANWELERTQQSGLERCEGDQDKRLHCYASWRNSSGT |
| | ActRIIB-ECD (SEQ ID NO: | IELVKKGCWLDDFNCYDRQECVATKENPQVYFCCCEGNFCNEKFSYFPQME |
| | 112) with 6 additional aa at | VTQPTSNPVTPKPP |
| | N-terminus | |
| 1582 | N19Q mutant of Hybrid hu- | AETRECIYYNANWELERTQQSGLERCEGEQDKRLHCYASWRNSSGTIEIVKK |
| | ActRIIB-ECD (SEQ ID NO: | GCWLDDFNCYDRQECVATKENPQVYFCCCEGNFCNEKFSYFPQMEVTQPTS |
| | 113) with 1 additional aa at | NPVTPKPP |
| | N-terminus | |
| 1583 | N20Q mutant of Hybrid hu- | EAETRECIYYNANWELERTQQSGLERCEGEQDKRLHCYASWRNSSGTIEIVK |
| | ActRIIB-ECD (SEQ ID NO: | KGCWLDDFNCYDRQECVATKENPQVYFCCCEGNFCNEKFSYFPQMEVTQP |
| | 113) with 2 additional aa at | TSNPVTPKPP |
| | N-terminus | |
| 1584 | N21Q mutant of Hybrid hu- | GEAETRECIYYNANWELERTQQSGLERCEGEQDKRLHCYASWRNSSGTIEIV |
| | ActRIIB-ECD (SEQ ID NO: | KKGCWLDDFNCYDRQECVATKENPQVYFCCCEGNFCNEKFSYFPQMEVTQ |
| | 113) with 3 additional aa at | PTSNPVTPKPP |
| | N-terminus | |
| 1585 | N22Q mutant of Hybrid hu- | RGEAETRECIYYNANWELERTQQSGLERCEGEQDKRLHCYASWRNSSGTIEI |
| | ActRIIB-ECD (SEQ ID NO: | VKKGCWLDDFNCYDRQECVATKENPQVYFCCCEGNFCNEKFSYFPQMEVT |
| | 113) with 4 additional aa at | QPTSNPVTPKPP |
| | N-terminus | |
| 1586 | N23Q mutant of Hybrid hu- | GRGEAETRECIYYNANWELERTQQSGLERCEGEQDKRLHCYASWRNSSGTI |
| | ActRIIB-ECD (SEQ ID NO: | EIVKKGCWLDDFNCYDRQECVATKENPQVYFCCCEGNFCNEKFSYFPQMEV |
| | 113) with 5 additional aa at | TQPTSNPVTPKPP |
| | N-terminus | |
| 1587 | N24Q mutant of Hybrid hu- | SGRGEAETRECIYYNANWELERTQQSGLERCEGEQDKRLHCYASWRNSSGT |
| | ActRIIB-ECD (SEQ ID NO: | IEIVKKGCWLDDFNCYDRQECVATKENPQVYFCCCEGNFCNEKFSYFPQME |
| | 113) with 6 additional aa at | VTQPTSNPVTPKPP |
| | N-terminus | |
| 1588 | N19Q mutant of Hybrid hu- | AETRECIYYNANWELERTQQSGLERCEGEQDKRLHCYASWRNSSGTIELVK |
| | ActRIIB-ECD (SEQ ID NO: | KGCWLDDFNCYDRQECVATKENPQVYFCCCEGNFCNEKFSYFPQMEVTQP |
| | 114) with 1 additional aa at | TSNPVTPKPP |
| | N-terminus | |
| 1589 | N20Q mutant of Hybrid hu- | EAETRECIYYNANWELERTQQSGLERCEGEQDKRLHCYASWRNSSGTIELV |
| | ActRIIB-ECD (SEQ ID NO: | KKGCWLDDFNCYDRQECVATKENPQVYFCCCEGNFCNEKFSYFPQMEVTQ |
| | 114) with 2 additional aa at | PTSNPVTPKPP |
| | N-terminus | |
| 1590 | N21Q mutant of Hybrid hu- | GEAETRECIYYNANWELERTQQSGLERCEGEQDKRLHCYASWRNSSGTIEL |
| | ActRIIB-ECD (SEQ ID NO: | VKKGCWLDDFNCYDRQECVATKENPQVYFCCCEGNFCNEKFSYFPQMEVT |
| | 114) with 3 additional aa at | QPTSNPVTPKPP |
| | N-terminus | |
| 1591 | N22Q mutant of Hybrid hu- | RGEAETRECIYYNANWELERTQQSGLERCEGEQDKRLHCYASWRNSSGTIE |
| | ActRIIB-ECD (SEQ ID NO: | LVKKGCWLDDFNCYDRQECVATKENPQVYFCCCEGNFCNEKFSYFPQMEV |
| | 114) with 4 additional aa at | TQPTSNPVTPKPP |
| | N-terminus | |
| 1592 | N23Q mutant of Hybrid hu- | GRGEAETRECIYYNANWELERTQQSGLERCEGEQDKRLHCYASWRNSSGTI |
| | ActRIIB-ECD (SEQ ID NO: | ELVKKGCWLDDFNCYDRQECVATKENPQVYFCCCEGNFCNEKFSYFPQME |
| | 114) with 5 additional aa at | VTQPTSNPVTPKPP |
| | N-terminus | |
| 1593 | N24Q mutant of Hybrid hu- | SGRGEAETRECIYYNANWELERTQQSGLERCEGEQDKRLHCYASWRNSSGT |
| | ActRIIB-ECD (SEQ ID NO: | IELVKKGCWLDDFNCYDRQECVATKENPQVYFCCCEGNFCNEKFSYFPQME |
| | 114) with 6 additional aa at | VTQPTSNPVTPKPP |
| | N-terminus | |
| 1594 | N19Q mutant of Hybrid hu- | AETRECIYYNANWELERTQQSGLERCEGDQDKRLHCYASWRNSSGTIEIVK |
| | ActRIIB-ECD (SEQ ID NO: | KGCWLDDFNCYDRQECVAKKENPQVYFCCCEGNFCNEKFSYFPQMEVTQP |
| | 115) with 1 additional aa at | TSNPVTPKPP |
| | N-terminus | |
| 1595 | N20Q mutant of Hybrid hu- | EAETRECIYYNANWELERTQQSGLERCEGDQDKRLHCYASWRNSSGTIEIVK |
| | ActRIIB-ECD (SEQ ID NO: | KGCWLDDFNCYDRQECVAKKENPQVYFCCCEGNFCNEKFSYFPQMEVTQP |
| | 115) with 2 additional aa at | TSNPVTPKPP |
| | N-terminus | |
| 1596 | N21Q mutant of Hybrid hu- | GEAETRECIYYNANWELERTQQSGLERCEGDQDKRLHCYASWRNSSGTIEIV |
| | ActRIIB-ECD (SEQ ID NO: | KKGCWLDDFNCYDRQECVAKKENPQVYFCCCEGNFCNEKFSYFPQMEVTQ |
| | 115) with 3 additional aa at | PTSNPVTPKPP |
| | N-terminus | |
| 1597 | N22Q mutant of Hybrid hu- | RGEAETRECIYYNANWELERTQQSGLERCEGDQDKRLHCYASWRNSSGTIEI |
| | ActRIIB-ECD (SEQ ID NO: | VKKGCWLDDFNCYDRQECVAKKENPQVYFCCCEGNFCNEKFSYFPQMEVT |
| | 115) with 4 additional aa at | QPTSNPVTPKPP |
| | N-terminus | |
| 1598 | N23Q mutant of Hybrid hu- | GRGEAETRECIYYNANWELERTQQSGLERCEGDQDKRLHCYASWRNSSGTI |
| | ActRIIB-ECD (SEQ ID NO: | EIVKKGCWLDDFNCYDRQECVAKKENPQVYFCCCEGNFCNEKFSYFPQME |
| | 115) with 5 additional aa at | VTQPTSNPVTPKPP |
| | N-terminus | |
| 1599 | N24Q mutant of Hybrid hu- | SGRGEAETRECIYYNANWELERTQQSGLERCEGDQDKRLHCYASWRNSSGT |
| | ActRIIB-ECD (SEQ ID NO: | IEIVKKGCWLDDFNCYDRQECVAKKENPQVYFCCCEGNFCNEKFSYFPQME |
| | 115) with 6 additional aa at | VTQPTSNPVTPKPP |
| | N-terminus | |
| 1600 | N19Q mutant of Hybrid hu- | AETQECLFFNANWEKDRTQQTGVEPCYGDKDKRRHCFATWKNISGSIEIVK |
| | ActRIIB-ECD (SEQ ID NO: | QGCWLDDINCYDRTDCVEKKDSPEVYFCCCEGNMCNERFTHLPEAGGPEVT |
| | 116) with 1 additional aa at | YEPPPTAPT |
| | N-terminus | |
| 1601 | N20Q mutant of Hybrid hu- | EAETQECLFFNANWEKDRTQQTGVEPCYGDKDKRRHCFATWKNISGSIEIV |
| | ActRIIB-ECD (SEQ ID NO: | KQGCWLDDINCYDRTDCVEKKDSPEVYFCCCEGNMCNERFTHLPEAGGPEV |
| | 116) with 2 additional aa at | TYEPPPTAPT |
| | N-terminus | |
| 1602 | N21Q mutant of Hybrid hu- | GEAETQECLFFNANWEKDRTQQTGVEPCYGDKDKRRHCFATWKNISGSIEI |
| | ActRIIB-ECD (SEQ ID NO: | VKQGCWLDDINCYDRTDCVEKKDSPEVYFCCCEGNMCNERFTHLPEAGGPE |
| | 116) with 3 additional aa at | VTYEPPPTAPT |
| | N-terminus | |
| 1603 | N22Q mutant of Hybrid hu- | RGEAETQECLFFNANWEKDRTQQTGVEPCYGDKDKRRHCFATWKNISGSIEI |
| | ActRIIB-ECD (SEQ ID NO: | VKQGCWLDDINCYDRTDCVEKKDSPEVYFCCCEGNMCNERFTHLPEAGGPE |
| | 116) with 4 additional aa at | VTYEPPPTAPT |
| | N-terminus | |
| 1604 | N23Q mutant of Hybrid hu- | GRGEAETQECLFFNANWEKDRTQQTGVEPCYGDKDKRRHCFATWKNISGSI |
| | ActRIIB-ECD (SEQ ID NO: | EIVKQGCWLDDINCYDRTDCVEKKDSPEVYFCCCEGNMCNERFTHLPEAGG |
| | 116) with 5 additional aa at | PEVTYEPPPTAPT |
| | N-terminus | |
| 1605 | N24Q mutant of Hybrid hu- | SGRGEAETQECLFFNANWEKDRTQQTGVEPCYGDKDKRRHCFATWKNISGS |
| | ActRIIB-ECD (SEQ ID NO: | IEIVKQGCWLDDINCYDRTDCVEKKDSPEVYFCCCEGNMCNERFTHLPEAG |
| | 116) with 6 additional aa at | GPEVTYEPPPTAPT |
| | N-terminus | |
| 1606 | N19Q mutant of Hybrid hu- | AETRECIYYNANWELERTQQSGLERCYGDKDKRRHCYASWRNSSGTIELVK |
| | ActRIIB-ECD (SEQ ID NO: | KGCWLDDINCYDRQECVATKENPQVYFCCCEGNFCNERFTHLPEAGGPEVT |
| | 117) with 1 additional aa at | YEPPPTAPT |
| | N-terminus | |
| 1607 | N20Q mutant of Hybrid hu- | EAETRECIYYNANWELERTQQSGLERCYGDKDKRRHCYASWRNSSGTIELV |
| | ActRIIB-ECD (SEQ ID NO: | KKGCWLDDINCYDRQECVATKENPQVYFCCCEGNFCNERFTHLPEAGGPEV |
| | 117) with 2 additional aa at | TYEPPPTAPT |
| | N-terminus | |
| 1608 | N21Q mutant of Hybrid hu- | GEAETRECIYYNANWELERTQQSGLERCYGDKDKRRHCYASWRNSSGTIEL |
| | ActRIIB-ECD (SEQ ID NO: | VKKGCWLDDINCYDRQECVATKENPQVYFCCCEGNFCNERFTHLPEAGGPE |
| | 117) with 3 additional aa at | VTYEPPPTAPT |
| | N-terminus | |
| 1609 | N22Q mutant of Hybrid hu- | RGEAETRECIYYNANWELERTQQSGLERCYGDKDKRRHCYASWRNSSGTIE |
| | ActRIIB-ECD (SEQ ID NO: | LVKKGCWLDDINCYDRQECVATKENPQVYFCCCEGNFCNERFTHLPEAGGP |
| | 117) with 4 additional aa at | EVTYEPPPTAPT |
| | N-terminus | |
| 1610 | N23Q mutant of Hybrid hu- | GRGEAETRECIYYNANWELERTQQSGLERCYGDKDKRRHCYASWRNSSGTI |
| | ActRIIB-ECD (SEQ ID NO: | ELVKKGCWLDDINCYDRQECVATKENPQVYFCCCEGNFCNERFTHLPEAGG |
| | 117) with 5 additional aa at | PEVTYEPPPTAPT |
| | N-terminus | |
| 1611 | N24Q mutant of Hybrid hu- | SGRGEAETRECIYYNANWELERTQQSGLERCYGDKDKRRHCYASWRNSSG |
| | ActRIIB-ECD (SEQ ID NO: | TIELVKKGCWLDDINCYDRQECVATKENPQVYFCCCEGNFCNERFTHLPEA |
| | 117) with 6 additional aa at | GGPEVTYEPPPTAPT |
| | N-terminus | |
| 1613 | L55D mutant of SEQ ID | AETRECIYYNANWELERTNQSGLERCEGEQDKRLHCYASWRNSSGTIELVK |
| | NO: 1 (SEQ ID NO: 327) | KGCWDDDFNCYDRQECVATEENPQVYFCCCEGNFCNERFTHLPEAGGPEVT |
| | with 1 additional aa at N- | YEPPPTAPT |
| | terminus | |
| 1614 | L55D mutant of SEQ ID | EAETRECIYYNANWELERTNQSGLERCEGEQDKRLHCYASWRNSSGTIELV |
| | NO: 1 (SEQ ID NO: 327) | KKGCWDDDFNCYDRQECVATEENPQVYFCCCEGNFCNERFTHLPEAGGPE |
| | with 2 additional aa at N- | VTYEPPPTAPT |
| | terminus | |
| 1615 | L55D mutant of SEQ ID | GEAETRECIYYNANWELERTNQSGLERCEGEQDKRLHCYASWRNSSGTIEL |
| | NO: 1 (SEQ ID NO: 327) | VKKGCWDDDFNCYDRQECVATEENPQVYFCCCEGNFCNERFTHLPEAGGP |
| | with 3 additional aa at N- | EVTYEPPPTAPT |
| | terminus | |
| 1616 | L55D mutant of SEQ ID | RGEAETRECIYYNANWELERTNQSGLERCEGEQDKRLHCYASWRNSSGTIE |
| | NO: 1 (SEQ ID NO: 327) | LVKKGCWDDDFNCYDRQECVATEENPQVYFCCCEGNFCNERFTHLPEAGG |
| | with 4 additional aa at N- | PEVTYEPPPTAPT |
| | terminus | |
| 1617 | L55D mutant of SEQ ID | GRGEAETRECIYYNANWELERTNQSGLERCEGEQDKRLHCYASWRNSSGTI |
| | NO: 1 (SEQ ID NO: 327) | ELVKKGCWDDDFNCYDRQECVATEENPQVYFCCCEGNFCNERFTHLPEAG |
| | with 5 additional aa at N- | GPEVTYEPPPTAPT |
| | terminus | |
| 1618 | L55D mutant of SEQ ID | SGRGEAETRECIYYNANWELERTNQSGLERCEGEQDKRLHCYASWRNSSGT |
| | NO: 1 (SEQ ID NO: 327) | IELVKKGCWDDDFNCYDRQECVATEENPQVYFCCCEGNFCNERFTHLPEAG |
| | with 6 additional aa at N- | GPEVTYEPPPTAPT |
| | terminus | |
| 1619 | N19Q mutant of SEQ ID | AETRECIYYNANWELERTQQSGLERCEGEQDKRLHCYASWRNSSGTIELVK |
| | NO: 327 with 1 additional aa | KGCWDDDFNCYDRQECVATEENPQVYFCCCEGNFCNERFTHLPEAGGPEVT |
| | at N-terminus | YEPPPTAPT |
| 1620 | N20Q mutant of SEQ ID | EAETRECIYYNANWELERTQQSGLERCEGEQDKRLHCYASWRNSSGTIELV |
| | NO: 327 with 2 additional aa | KKGCWDDDFNCYDRQECVATEENPQVYFCCCEGNFCNERFTHLPEAGGPE |
| | at N-terminus | VTYEPPPTAPT |
| 1621 | N21Q mutant of SEQ ID | GEAETRECIYYNANWELERTQQSGLERCEGEQDKRLHCYASWRNSSGTIEL |
| | NO: 327 with 3 additional aa | VKKGCWDDDFNCYDRQECVATEENPQVYFCCCEGNFCNERFTHLPEAGGP |
| | at N-terminus | EVTYEPPPTAPT |
| 1622 | N22Q mutant of SEQ ID | RGEAETRECIYYNANWELERTQQSGLERCEGEQDKRLHCYASWRNSSGTIE |
| | NO: 327 with 4 additional aa | LVKKGCWDDDFNCYDRQECVATEENPQVYFCCCEGNFCNERFTHLPEAGG |
| | at N-terminus | PEVTYEPPPTAPT |
| 1623 | N23Q mutant of SEQ ID | GRGEAETRECIYYNANWELERTQQSGLERCEGEQDKRLHCYASWRNSSGTI |
| | NO: 327 with 5 additional aa | ELVKKGCWDDDFNCYDRQECVATEENPQVYFCCCEGNFCNERFTHLPEAG |
| | at N-terminus | GPEVTYEPPPTAPT |
| 1624 | N24Q mutant of SEQ ID | SGRGEAETRECIYYNANWELERTQQSGLERCEGEQDKRLHCYASWRNSSGT |
| | NO: 327 with 6 additional aa | IELVKKGCWDDDFNCYDRQECVATEENPQVYFCCCEGNFCNERFTHLPEAG |
| | at N-terminus | GPEVTYEPPPTAPT |
| 1626 | L55E mutant of SEQ ID NO: | AETRECIYYNANWELERTNQSGLERCEGEQDKRLHCYASWRNSSGTIELVK |
| | 1 (SEQ ID NO: 330) with 1 | KGCWEDDFNCYDRQECVATEENPQVYFCCCEGNFCNERFTHLPEAGGPEVT |
| | additional aa at N-terminus | YEPPPTAPT |
| 1627 | L55E mutant of SEQ ID NO: | EAETRECIYYNANWELERTNQSGLERCEGEQDKRLHCYASWRNSSGTIELV |
| | 1 (SEQ ID NO: 330) with 2 | KKGCWEDDFNCYDRQECVATEENPQVYFCCCEGNFCNERFTHLPEAGGPEV |
| | additional aa at N-terminus | TYEPPPTAPT |
| 1628 | L55E mutant of SEQ ID NO: | GEAETRECIYYNANWELERTNQSGLERCEGEQDKRLHCYASWRNSSGTIEL |
| | 1 (SEQ ID NO: 330) with 3 | VKKGCWEDDFNCYDRQECVATEENPQVYFCCCEGNFCNERFTHLPEAGGPE |
| | additional aa at N-terminus | VTYEPPPTAPT |
| 1629 | L55E mutant of SEQ ID NO: | RGEAETRECIYYNANWELERTNQSGLERCEGEQDKRLHCYASWRNSSGTIE |
| | 1 (SEQ ID NO: 330) with 4 | LVKKGCWEDDFNCYDRQECVATEENPQVYFCCCEGNFCNERFTHLPEAGGP |
| | additional aa at N-terminus | EVTYEPPPTAPT |
| 1630 | L55E mutant of SEQ ID NO: | GRGEAETRECIYYNANWELERTNQSGLERCEGEQDKRLHCYASWRNSSGTI |
| | 1 (SEQ ID NO: 330) with 5 | ELVKKGCWEDDFNCYDRQECVATEENPQVYFCCCEGNFCNERFTHLPEAGG |
| | additional aa at N-terminus | PEVTYEPPPTAPT |
| 1631 | L55E mutant of SEQ ID NO: | SGRGEAETRECIYYNANWELERTNQSGLERCEGEQDKRLHCYASWRNSSGT |
| | 1 (SEQ ID NO: 330) with 6 | IELVKKGCWEDDFNCYDRQECVATEENPQVYFCCCEGNFCNERFTHLPEAG |
| | additional aa at N-terminus | GPEVTYEPPPTAPT |
| 1632 | N19Q mutant of SEQ ID | AETRECIYYNANWELERTQQSGLERCEGEQDKRLHCYASWRNSSGTIELVK |
| | NO: 330 with 1 additional aa | KGCWEDDFNCYDRQECVATEENPQVYFCCCEGNFCNERFTHLPEAGGPEVT |
| | at N-terminus | YEPPPTAPT |
| 1633 | N20Q mutant of SEQ ID | EAETRECIYYNANWELERTQQSGLERCEGEQDKRLHCYASWRNSSGTIELV |
| | NO: 330 with 2 additional aa | KKGCWEDDFNCYDRQECVATEENPQVYFCCCEGNFCNERFTHLPEAGGPEV |
| | at N-terminus | TYEPPPTAPT |
| 1634 | N21Q mutant of SEQ ID | GEAETRECIYYNANWELERTQQSGLERCEGEQDKRLHCYASWRNSSGTIEL |
| | NO: 330 with 3 additional aa | VKKGCWEDDFNCYDRQECVATEENPQVYFCCCEGNFCNERFTHLPEAGGPE |
| | at N-terminus | VTYEPPPTAPT |
| 1635 | N22Q mutant of SEQ ID | RGEAETRECIYYNANWELERTQQSGLERCEGEQDKRLHCYASWRNSSGTIE |
| | NO: 330 with 4 additional aa | LVKKGCWEDDFNCYDRQECVATEENPQVYFCCCEGNFCNERFTHLPEAGGP |
| | at N-terminus | EVTYEPPPTAPT |
| 1636 | N23Q mutant of SEQ ID | GRGEAETRECIYYNANWELERTQQSGLERCEGEQDKRLHCYASWRNSSGTI |
| | NO: 330 with 5 additional aa | ELVKKGCWEDDFNCYDRQECVATEENPQVYFCCCEGNFCNERFTHLPEAGG |
| | at N-terminus | PEVTYEPPPTAPT |
| 1637 | N24Q mutant of SEQ ID | SGRGEAETRECIYYNANWELERTQQSGLERCEGEQDKRLHCYASWRNSSGT |
| | NO: 330 with 6 additional aa | IELVKKGCWEDDFNCYDRQECVATEENPQVYFCCCEGNFCNERFTHLPEAG |
| | at N-terminus | GPEVTYEPPPTAPT |
| 1639 | R40A mutant of SEQ ID | AETRECIYYNANWELERTNQSGLERCEGEQDKRLHCYASWANSSGTIELVK |
| | NO: 1 (SEQ ID NO: 333) | KGCWLDDFNCYDRQECVATEENPQVYFCCCEGNFCNERFTHLPEAGGPEVT |
| | with 1 additional aa at N- | YEPPPTAPT |
| | terminus | |
| 1640 | R40A mutant of SEQ ID | EAETRECIYYNANWELERTNQSGLERCEGEQDKRLHCYASWANSSGTIELV |
| | NO: 1 (SEQ ID NO: 333) | KKGCWLDDFNCYDRQECVATEENPQVYFCCCEGNFCNERFTHLPEAGGPEV |
| | with 2 additional aa at N- | TYEPPPTAPT |
| | terminus | |
| 1641 | R40A mutant of SEQ ID | GEAETRECIYYNANWELERTNQSGLERCEGEQDKRLHCYASWANSSGTIEL |
| | NO: 1 (SEQ ID NO: 333) | VKKGCWLDDFNCYDRQECVATEENPQVYFCCCEGNFCNERFTHLPEAGGPE |
| | with 3 additional aa at N- | VTYEPPPTAPT |
| | terminus | |
| 1642 | R40A mutant of SEQ ID | RGEAETRECIYYNANWELERTNQSGLERCEGEQDKRLHCYASWANSSGTIE |
| | NO: 1 (SEQ ID NO: 333) | LVKKGCWLDDFNCYDRQECVATEENPQVYFCCCEGNFCNERFTHLPEAGGP |
| | with 4 additional aa at N- | EVTYEPPPTAPT |
| | terminus | |
| 1643 | R40A mutant of SEQ ID | GRGEAETRECIYYNANWELERTNQSGLERCEGEQDKRLHCYASWANSSGTI |
| | NO: 1 (SEQ ID NO: 333) | ELVKKGCWLDDFNCYDRQECVATEENPQVYFCCCEGNFCNERFTHLPEAGG |
| | with 5 additional aa at N- | PEVTYEPPPTAPT |
| | terminus | |
| 1644 | R40A mutant of SEQ ID | SGRGEAETRECIYYNANWELERTNQSGLERCEGEQDKRLHCYASWANSSGT |
| | NO: 1 (SEQ ID NO: 333) | IELVKKGCWLDDFNCYDRQECVATEENPQVYFCCCEGNFCNERFTHLPEAG |
| | with 6 additional aa at N- | GPEVTYEPPPTAPT |
| | terminus | |
| 1645 | N19Q mutant of SEQ ID | AETRECIYYNANWELERTQQSGLERCEGEQDKRLHCYASWANSSGTIELVK |
| | NO: 333 with 1 additional aa | KGCWLDDFNCYDRQECVATEENPQVYFCCCEGNFCNERFTHLPEAGGPEVT |
| | at N-terminus | YEPPPTAPT |
| 1646 | N19Q mutant of SEQ ID | EAETRECIYYNANWELERTQQSGLERCEGEQDKRLHCYASWANSSGTIELV |
| | NO: 333 with 2 additional aa | KKGCWLDDFNCYDRQECVATEENPQVYFCCCEGNFCNERFTHLPEAGGPEV |
| | at N-terminus | TYEPPPTAPT |
| 1647 | N19Q mutant of SEQ ID | GEAETRECIYYNANWELERTQQSGLERCEGEQDKRLHCYASWANSSGTIEL |
| | NO: 333 with 3 additional aa | VKKGCWLDDFNCYDRQECVATEENPQVYFCCCEGNFCNERFTHLPEAGGPE |
| | at N-terminus | VTYEPPPTAPT |
| 1648 | N19Q mutant of SEQ ID | RGEAETRECIYYNANWELERTQQSGLERCEGEQDKRLHCYASWANSSGTIE |
| | NO: 333 with 4 additional aa | LVKKGCWLDDFNCYDRQECVATEENPQVYFCCCEGNFCNERFTHLPEAGGP |
| | at N-terminus | EVTYEPPPTAPT |
| 1649 | N19Q mutant of SEQ ID | GRGEAETRECIYYNANWELERTQQSGLERCEGEQDKRLHCYASWANSSGTI |
| | NO: 333 with 5 additional aa | ELVKKGCWLDDFNCYDRQECVATEENPQVYFCCCEGNFCNERFTHLPEAGG |
| | at N-terminus | PEVTYEPPPTAPT |
| 1650 | N19Q mutant of SEQ ID | SGRGEAETRECIYYNANWELERTQQSGLERCEGEQDKRLHCYASWANSSGT |
| | NO: 333 with 6 additional aa | IELVKKGCWLDDFNCYDRQECVATEENPQVYFCCCEGNFCNERFTHLPEAG |
| | at N-terminus | GPEVTYEPPPTAPT |

Various modifications and variations of the described methods, compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

All publications, published patent documents, and patent applications cited herein are hereby incorporated by reference to the same extent as though each individual publication, published patent document, or patent application was specifically and individually indicated as being incorporated by reference.

What is claimed is:

1. An isolated protein comprising a mutant soluble activin IIB receptor (ActRIIB) extracellular domain (ActRIIB-ECD), wherein said mutant soluble ActRIIB-ECD comprises a mutation to remove the N-linked glycosylation site corresponding to position N18 of SEQ ID NO: 1.

2. The isolated protein of claim 1, wherein the mutant soluble ActRIIB-ECD further comprises a substitution of at least one of amino acid residues R3, I6, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 1 with another amino acid.

3. The isolated protein of claim 1, wherein the mutant soluble ActRIIB-ECD comprises an amino acid sequence at least 95% identical to an amino acid sequence selected from the group consisting of SEQ ID NOs: 1, 3-37, 51-117, 327, 330, 333, and 1658, wherein the asparagine at position N18 is substituted with another amino acid.

4. The isolated protein of claim 1, wherein the mutant soluble ActRIIB-ECD comprises an amino acid sequence selected from the group consisting of SEQ ID NOs: 1, 3-37, 51-117, 327, 330, 333, and 1658, wherein the asparagine at position N18 is substituted with another amino acid.

5. The isolated protein of claim 1, wherein the serine at position S20 of SEQ ID NO: 1 is substituted with an amino acid that is not serine(S) or threonine (T).

6. The isolated protein of any one or combination of claims 1-5, wherein the asparagine at position N18 is substituted with glutamine (Q).

7. The isolated protein of claim 1, wherein the mutant soluble ActRIIB-ECD comprises an amino acid sequence selected from the group consisting of SEQ ID NOs: 119, 120-221, 329, 332, 335, and 1660.

8. The isolated protein of claim 1, wherein the mutant soluble ActRIIB-ECD comprises an amino acid sequence selected from the group consisting of SEQ ID NOs: 336-354.

9. The isolated protein of claim 1, wherein the mutant soluble ActRIIB-ECD comprises an amino acid sequence selected from the group consisting of SEQ ID NOs: 355-372.

10. The isolated protein of any one or combination of claims 1-9, wherein said mutant soluble ActRIIB-ECD demonstrates increased binding of myostatin relative to an otherwise identical soluble ActRIIB-ECD that includes the N-linked glycosylation site corresponding to position N18 of SEQ ID NO: 1.

11. The isolated protein of any one or combination of claims 1-10, wherein said mutant soluble ActRIIB-ECD is glycosylated at an asparagine residue corresponding to position N41 of SEQ ID NO: 1.

12. The isolated protein of claim 11, wherein the glycosylated mutant soluble ActRIIB-ECD is sialylated.

13. The isolated protein of claim 11, wherein a sample of said mutant soluble ActRIIB-ECD comprises at least 40% sialylated glycans.

14. The isolated protein of any one or combination of claims 1-13, wherein the mutant soluble ActRIIB-ECD is fused to at least one heterologous protein.

15. The isolated protein of claim 14, wherein the heterologous protein comprises a constant domain of an immunoglobulin.

16. The isolated protein of any one or combination of claims 14-15, wherein the heterologous protein comprises an Fc domain of an immunoglobulin.

17. The isolated protein of claim 16, wherein the Fc domain is selected from the group consisting of the Fc domain of a human immunoglobulin gamma-1 (IgG1), the Fc domain of a human immunoglobulin gamma-2 (IgG2), and the Fc domain of a human immunoglobulin gamma-4 (IgG4).

18. The isolated protein of any one or combination of claims 1-17, wherein the mutant soluble ActRIIB-ECD is fused to the heterologous protein by a peptide linker sequence.

19. The isolated protein of any one or combination of claims 14-18, wherein the heterologous protein comprises a human Fc domain comprising an amino acid sequence selected from the group consisting of: SEQ ID NO: 39, SEQ ID NO: 41, and SEQ ID NO: 43.

20. The isolated protein of any one or combination of claims 1-19, wherein the isolated protein comprises a linker comprising the amino acid sequence set forth in SEQ ID NO: 44 between the mutant soluble ActRIIB-ECD and the heterologous protein.

21. The isolated protein of any one or combination of claims 1-20, wherein the isolated protein comprises a hinge linker comprising the amino acid sequence set forth in SEQ ID NO: 118 between the mutant soluble ActRIIB-ECD and the heterologous protein.

22. The isolated protein of any one or combination of claims 1-21, wherein the isolated protein comprises a linker comprising the amino acid sequence set forth in SEQ ID NO: 44 and a hinge linker comprising the amino acid sequence set forth in SEQ ID NO: 118 between the mutant soluble ActRIIB-ECD and the heterologous protein.

23. The isolated protein of any one or combination of claims 1-22, wherein the isolated protein comprises, in an N-terminal to C-terminal direction, the mutant soluble ActRIIB-ECD, the linker of SEQ ID NO: 44, the hinge linker of SEQ ID NO: 118, and the heterologous protein.

24. The isolated protein of any one or combination of claims 1-23, wherein the mutant soluble ActRIIB-ECD comprises an amino acid sequence selected from the group consisting of SEQ ID NOs: 119, 120-221, 329, 332,335, 336-354, 355-372, and 1660 and wherein the heterologous protein is selected from the group consisting of the Fc domain of a human immunoglobulin gamma-1 (IgG1), the Fc domain of a human immunoglobulin gamma-2 (IgG2), and the Fc domain of a human immunoglobulin gamma-4 (IgG4).

25. The isolated protein of claim 24, wherein the mutant soluble ActRIIB-ECD comprises SEQ ID NO: 146 and the heterologous protein comprises the Fc domain of a human IgG4.

26. The isolated protein of claim 1, wherein the isolated protein comprises SEQ ID NO: 222 or 1664.

27. The isolated protein of claim 1, wherein the isolated protein consists of SEQ ID NO: 222 or 1664.

28. The isolated protein of any one or combination of claims 24-27, wherein said isolated protein is glycosylated at an asparagine residue in the mutant soluble ActRIIB-ECD corresponding to positions N41 of SEQ ID NO: 1 and/or an asparagine residue in the Fc domain corresponding to position N67 of SEQ ID NO: 43.

29. The isolated protein of claim 28, wherein a sample of said isolated protein comprises at least 40% sialylated glycans.

30. A pharmaceutical composition comprising a therapeutically effective amount of the isolated protein of any one of claims 1-29 in admixture with a pharmaceutically acceptable carrier.

31. The pharmaceutical composition of claim 30, wherein the pharmaceutical composition is formulated for administration by a route selected from the group consisting of:
subcutaneous, intramuscular, intravenous, and intrathecal administration.

32. The pharmaceutical composition of claim 30 or 31, wherein said pharmaceutical composition further comprises a second agent, wherein said second agent is selected from the group consisting of: growth hormone, ghrelin, IGF1, insulin, prednisone, corticosteroid therapy, androgen-deprivation therapy, anabolic steroids, an antagonist of angiotensin or angiotensin receptor, an antagonist of an inflammatory cytokine such as TNF-alpha, IL-6, IL-1 or their receptors, an antagonist of myostatin, activin A or another member of the TGF-beta family or their receptors, bisphosphonates, RANKL inhibitors, agonists of peroxisome proliferator-activated receptors, β2 agonists, activator of PGC-1 alpha, proteasome inhibitors, a cancer therapeutic, a chemotherapeutic agent, a cell therapy, a stem cell therapy, gene therapy, gene targeting therapy, and an antisense oligonucleotide.

33. The pharmaceutical composition of any one or combination of claims 30-32, wherein the pharmaceutical composition comprises a plurality of the isolated proteins and at least 40% of the isolated proteins are sialylated.

34. A sample comprising a plurality of the isolated proteins of any one or combination of claims 1-29, wherein at least 40% of the isolated proteins are sialylated.

35. A method of treating a myostatin-related or activin A-related disorder in a subject in need thereof, comprising administering a therapeutically effective amount of the pharmaceutical composition of any one of claims 30-33 to the subject.

36. The method of claim 35, wherein said myostatin-related or activin A-related disorder is selected from the group consisting of muscle wasting, a bone disorder, a metabolic disorder, and anemia.

37. The method of claim 36, wherein the muscle wasting is associate with a condition selected from the group consisting of: muscular dystrophy, myositis, myopathy, motorneuron disease, muscle atrophy, amyotrophic lateral sclerosis, spinal muscular atrophy, neuromuscular junction disease, peripheral nerve disease, spinal cord injury, stroke, neurodegenerative disease, anorexia, cancer, organ failure, trauma, disuse, infection, chronic obstructive pulmonary disease (COPD), sarcopenia, sarcopenic obesity, osteroarthritis, androgen deprivation, emphysema, cystic fibrosis, chronic heart failure, cardiac atrophy, cancer cachexia, renal failure, uremia, protein energy wasting, anorexia, malnutrition, sarcopenia, Acquired Immunodeficiency Syndrome (AIDS), sepsis, burn injury, diabetes, carpal tunnel syndrome, prolonged bed rest, bone fracture, aging, and exposure to microgravity.

38. The method of claim 37, wherein said spinal muscular atrophy is selected from the group consisting of infantile progressive spinal muscular atrophy, intermediate spinal muscular atrophy, juvenile spinal muscular atrophy and adult spinal muscular atrophy.

39. The method of claim 37, wherein said peripheral nerve disease is selected from the group consisting of Charcot-Marie Tooth disease, Dejerine-Sottas disease and Friedreich's ataxia.

40. The method of claim 37, wherein said neurodegenerative disease is selected from the group consisting of Parkinson's disease, Huntington's disease, Alzheimer's disease and Creutzfeldt-Jakob disease.

41. The method of claim 37, wherein said aging condition is selected from the group consisting of: frailty of the elderly, age-related sarcopenia, and osteoarthritis.

42. The method of claim 37, wherein said motorneuron disease is amyotrophic lateral sclerosis.

43. The method of claim 37, wherein said myopathy is critical illness myopathy or intensive care unit (ICU) myopathy.

44. The method of claim 37, wherein said muscular dystrophy is myotonic dystrophy type 1 (DM1), Facioscapulohumeral muscular dystrophy (FSHD), Limb-girdle muscular dystrophies (LGMD), or Duchenne muscular dystrophy (DMD).

45. The method of claim 36, wherein said bone disorder is selected from the group consisting of: osteoporosis, renal osteodystrophy, osteomalacia, osteogenesis imperfecta, fibrodysplasia ossificans progressiva, corticosteroid-induced bone loss, androgen-deprivation therapy-induced bone loss, bone fracture, cancer-induced bone loss, bone metastasis, Paget's disease of the bone, Rickets, Perthes' disease and fibrous dysplasia.

46. The method of any one or combination of claims 35-45, wherein the method further comprises administering a second agent to the subject in need thereof, wherein the second agent is administered prior to, concurrently with, or subsequent to administration of the pharmaceutical composition.

47. A polynucleotide encoding a protein comprising:
a. a mutant soluble ActRIIB-ECD sequence selected from the group consisting of SEQ ID NOs: 119, 120-221, 329, 332, 335, 336-354, 355-372, and 1660, and
b. an Fc domain sequence of a human IgG.

48. A polynucleotide of claim 47, wherein the protein further comprises:
a. the peptide linker sequence of SEQ ID NO: 44;
b the hinge linker sequence of SEQ ID NO: 118; or
c. both the peptide linker sequence of SEQ ID NO: 44 and the hinge linker sequence of SEQ ID NO: 118.

49. A polynucleotide of claim 47 or 48, wherein the polynucleotide further comprises a signal peptide sequence.

50. A polynucleotide comprising a DNA sequence of SEQ ID NO: 1653.

51. A vector comprising the polynucleotide of any of claims 47-50.

52. A host cell comprising the polynucleotide of any claims 47-50.

53. The host cell of claim 52, wherein the host cell is a mammalian cell.

54. A method of producing a protein comprising a mutant soluble ActRIIB-ECD comprising culturing the host cell of claim 52 or 53 under conditions promoting the expression of the protein, and recovering the protein.

55. The method of claim 54, wherein the method further comprises purifying the protein using one or more of Protein A chromatography, size exclusion chromatography, or ion exchange chromatography.
